(19) United States
(12) Patent Application Publication
(10) Pub. No.: US 2024/0360103 A1
(43) Pub. Date: Oct. 31, 2024

(54) COMPLEMENT INHIBITION

(71) Applicant: Apellis Pharmaceuticals, Inc., Waltham, MA (US)

(72) Inventors: Johannes Wilhelm Georg Meissner, Groningen (NL); Andrew Simon Cook, Stow, MA (US); Mark William Orme, Seattle, WA (US)

(21) Appl. No.: 18/573,634

(22) PCT Filed: Jun. 30, 2022
(86) PCT No.: PCT/US22/35717

§ 371 Date: Dec. 22, 2023

Related U.S. Application Data
(60) Provisional application No. 63/217,173, filed on Jun. 30, 2021.

(51) Int. Cl. C07D 401/14; A61K 31/496; A61K 31/501; A61K 31/506; C07D 403/06; C07D 403/14; C07D 405/14; C07D 413/14; C07D 417/14

(52) U.S. Cl. CPC C07D 401/14; A61K 31/496; A61K 31/501; A61K 31/506; C07D 403/06; C07D 403/14; C07D 405/14; C07D 413/14; C07D 417/14

(57) ABSTRACT

Among other things, the present disclosure provides technologies for modulating complement activation. In some embodiments, the present disclosure provides compounds that inhibit C3 convertase activity. In some embodiments, the present disclosure provides methods for treating complement-mediated conditions, disorders or diseases. In some embodiments, the present disclosure provides methods for treating conditions, disorders or diseases mediated by C3 convertase.

### CROSS-REFERENCE TO RELATED APPLICATIONS

This application claims priority to U.S. Provisional Application No. 63/217,173, filed Jun. 30, 2021, the entirety of which is incorporated herein by reference.

### BACKGROUND

Complement is a system consisting of more than 30 plasma and cell-bound proteins that plays a significant role in both innate and adaptive immunity. The proteins of the complement system act in a series of enzymatic cascades through a variety of protein interactions and cleavage events. Complement activation occurs via three main pathways: the antibody-dependent classical pathway, the alternative pathway, and the mannose-binding lectin (MBL) pathway. Inappropriate or excessive complement activation is an underlying cause or contributing factor to a number of serious diseases and conditions, and considerable effort has been devoted over the past several decades to exploring various complement inhibitors as therapeutic agents.

### SUMMARY

Among other things, the present disclosure provides technologies (e.g., compounds, compositions, methods, etc.) useful for treating various conditions, disorders or diseases. In some embodiments, the present disclosure provides technologies for preventing or treating conditions, disorders or diseases associated with complement activation. In some embodiments, a condition, disorder or disease is associated with alternative complement activation. In some embodiments, a condition, disorder or disease is associated with C3 convertase. In some embodiments, a condition, disorder or disease is associated with factor B.

In some embodiments, the present disclosure provides a compound having the structure of formula I:

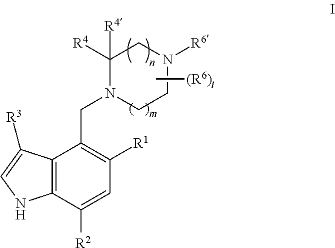

or a pharmaceutically acceptable salt thereof, wherein:
m is 0, 1, or 2;
n is 1, or 2;
R¹ is hydrogen, halogen, hydroxy, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷, phenyl or 5- or 6-membered heteroaryl having 1, 2 or 3 ring heteroatoms independently selected from nitrogen, oxygen and sulfur, wherein the phenyl or heteroaryl is optionally substituted with 0, 1, or 2 C₁-C₄ alkyl groups, and wherein alkyl and haloalkyl are optionally substituted with 0 or 1 hydroxy;
R⁴ is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl;
R^{4′} is hydrogen, C₁-C₄ alkyl, or hydroxy C₁-C₄ alkyl;
R⁵ is —C(O)R⁸, —CH₂C(O)R⁸, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
R⁷ is hydroxy, C₁-C₄ alkoxy, amino, or mono- or di-C₁-C₄ alkylamino;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.

Among other things, provided compounds, e.g., those of formula I or a pharmaceutically acceptable salt thereof, are particularly useful for modulating C3 convertase activity. In some embodiments, provided compounds are useful for modulating complement activation. In some embodiments, the present disclosure provides a method for modulating a C3 convertase activity, comprising contacting a C3 convertase with a provided compound. In some embodiments, the present disclosure provides a method for modulating a C3 convertase activity, comprising administering to a system comprising a C3 convertase a provided compound. In some embodiments, the present disclosure provides a method for modulating a C3 convertase activity, comprising administering to a subject expressing or comprising a C3 convertase a provided compound. In some embodiments, an activity of a C3 convertase is inhibited. In some embodiments, a provided compound is useful as a C3 convertase inhibitor. In some embodiments, a C3 convertase is or comprises factor B. In some embodiments, a C3 convertase is factor B. In some embodiments, a C3 convertase forms a complex with one or more polypeptides.

In some embodiments, the present disclosure provides methods for preventing a condition, disorder or disease, comprising administering to a subject susceptible thereto an effective amount of a provided compound (e.g., a compound of formula I or a pharmaceutically acceptable salt thereof).

In some embodiments, the present disclosure provides methods for treating a condition, disorder or disease, comprising administering to a subject suffering therefrom an effective amount of a provided compound (e.g., a compound of formula I or a pharmaceutically acceptable salt thereof).

Various conditions, disorders or diseases associated with complement activation can prevented or treated utilizing provided technologies in accordance with the present disclosure. In some embodiments, a condition, disorder or disease is associated with complement activation. In some embodiments, a condition, disorder or disease is associated with alternative complement activation. In some embodiments, a condition, disorder or disease is associated with C3 convertase. In some embodiments, a subject who is suffering from a condition, disorder or disease can benefit from inhibition of a C3 convertase.

In some embodiments, a condition, disorder or disease is selected from age-related macular degeneration (e.g., intermediate age-related macular degeneration), geographic atrophy, Stargardt's disease, diabetic retinopathy, uveitis, glaucoma, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Creutzfeld-Jacob disease, Guillain Barre Syndrome, spinal cord injury, traumatic brain injury, Alzheimer's disease, Parkinson's disease, progressive supranuclear palsy, corticobasal syndrome, Pick's disease, mild cognitive impairment, Huntington's disease, diabetic neuropathy, neuropathic pain syndromes, fibromyalgia, frontotemporal dementia, dementia with Lewy bodies, multiple system atrophy, leptomeningeal metastasis, amyotrophic lateral sclerosis (ALS), chronic inflammatory demyelinating polyneuropathy (CIDP), neuromyelitis optica (NMO), disorders of inappropriate or undesirable complement activation, hemodialysis complications, graft rejection (e.g., hyperacute allograft rejection, xenograft rejection, interleukin-2 induced toxicity during IL-2 therapy, inflammatory disorders, inflammation of autoimmune diseases, Crohn's disease, acute respiratory distress syndrome (ARDS), myocarditis, postischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, asthma, chronic obstructive pulmonary disease (COPD), emphysema, pulmonary embolisms and infarcts, pneumonia, fibrogenic dust diseases, pulmonary fibrosis, rhinosinusitis, nasal polyposis, allergy, bronchoconstriction, hypersensitivity pneumonitis, parasitic diseases, Goodpasture's Syndrome, pulmonary vasculitis, pauci-immune vasculitis, thrombotic microangiopathy (TMA), immune complex-associated inflammation, antiphospholipid syndrome, glomerulonephritis, C3 glomerulopathy, IgA nephropathy, cancer, periodontitis, gingivitis, and obesity.

In some embodiments a subject with hemolytic anemia suffers from paroxysmal nocturnal hemoglobinuria. In some embodiments a subject with hemolytic anemia suffers from autoimmune hemolytic anemia (e.g., cold agglutinin disease or warm autoimmune hemolytic anemia). In some embodiments a subject suffers from TMA secondary to atypical hemolytic uremic syndrome.

In some embodiments, a condition, disorder or disease, e.g., a complement-mediated disorder, is complement activation secondary to administration of another agent, e.g., a therapeutic or diagnostic agent. For example, in some embodiments, a complement-mediated disorder is complement activation secondary to gene therapy (e.g., gene therapy with a viral vector such as an adeno-associated virus (AAV), adenovirus, or lentivirus vector) or complement activation secondary to cell therapy. In some embodiments, a subject suffers from TMA secondary to hematopoietic stem cell transplant (HSCT-TMA). In some embodiments, a subject suffers from drug-induced TMA. In some embodiments, administration of a compound described herein prior to and/or following administration of another therapeutic agent may increase the efficacy and/or safety of said therapeutic agent. In some embodiments, the present disclosure provides methods for improving efficacy and/or safety of a therapeutic agent, comprising administering to a subject an effective amount of a provided compound (e.g., a compound of formula I or a pharmaceutically acceptable salt thereof) prior to, concurrently with (either in the same or different composition) or subsequently to administration of the therapeutic agent to the subject. In some embodiments, the present disclosure provides improved administration (e.g., dosage regimen, unit doses, total doses, improved intervals, durations of treatment, etc.) of a therapeutic agent, comprising administering to a subject a provided compound (e.g., a compound of formula I or a pharmaceutically acceptable salt thereof) prior to, concurrently with (either in the same or different composition) or subsequently to administration of the therapeutic agent to the subject, wherein the administration of the therapeutic agent provides improved efficacy and/or safety compared to a reference administration (e.g., administration of the therapeutic agent without administration of a provided compound (e.g., a compound of formula I or a pharmaceutically acceptable salt thereof)).

In some embodiments, a subject has a defect in complement regulation, optionally wherein the defect comprises abnormally low expression of one or more complement regulatory proteins by at least some of the subject's cells.

In some embodiments, a complement-mediated disorder is a chronic disorder. In some embodiments, a complement-mediated disorder involves complement-mediated damage to red blood cells, optionally wherein the disorder is paroxysmal nocturnal hemoglobinuria or atypical hemolytic uremic syndrome. In some embodiments, a complement-mediated disorder is an autoimmune disease, optionally wherein the disorder is multiple sclerosis. In some embodiments, a complement-mediated disorder involves kidney, optionally wherein the disorder is membranoproliferative glomerulonephritis, lupus nephritis, IgA nephropathy (IgAN), primary membranous nephropathy (primary MN), C3 glomerulopathy (C3G), or acute kidney injury. In some embodiments, a complement-mediated disorder involves the central or peripheral nervous system or neuromuscular junction, optionally wherein a disorder is neuromyelitis optica, Guillain-Barre syndrome, multifocal motor neuropathy, or myasthenia gravis. In some embodiments, a complement-mediated disorder involves the respiratory system, optionally wherein the disorder is characterized by pulmonary fibrosis. In some embodiments, a complement-mediated disorder involves the vascular system, optionally wherein the disorder is characterized by vasculitis.

Certain conditions, disorders or diseases that can be prevented or treated by provided technologies, e.g., conditions, disorders or diseases mediated by complement activation (in some embodiments, alternative complement activation), conditions, disorders or diseases mediated by C3 convertase, conditions, disorders or diseases mediated by factor B, etc. are reported in, e.g., WO 2015/009616, WO 2019/043609, etc. In some embodiments, a condition, disorder or disease is a renal condition, disorder or disease. In some embodiments, a condition, disorder or disease is complement-driven renal disease C3G (C3 glomerulopathy). In some embodiments, a condition, disorder or disease is c IgAN (immunoglobuline A nephropathy). In some embodiments, a condition, disorder or disease is a nephropathy. In some embodiments, a condition, disorder or disease is a nephropathy with evidence of glomerular C3 deposition. In some embodiments, a condition, disorder or disease is membranous nephropathy. In some embodiments, a condition, disorder or disease is HUS (E. coli induced hemolytic uremic syndrome).

In some embodiments, a compound (e.g., a compound of formula I or a pharmaceutically acceptable salt thereof) is provided in a pharmaceutical composition. In some embodiments, the present disclosure provides a pharmaceutical composition comprising a provided compound and a pharmaceutically acceptable carrier. In some embodiments, the present disclosure provides a pharmaceutical composition comprising a compound of formula I or a pharmaceutically acceptable salt thereof and a pharmaceutically acceptable carrier. In some embodiments, a composition may comprise two or more pharmaceutically acceptable salt forms of a compound. In some embodiments, a composition comprises two or more pharmaceutically acceptable salts of a compound of formula I. In some embodiments, the present disclosure provides a pharmaceutical composition which delivers a provided compound (e.g., a compound of formula I or a pharmaceutically acceptable salt thereof) and comprises a pharmaceutically acceptable carrier. In some embodiments, the present disclosure provides a pharmaceutical composition comprises a compound of formula I or a pharmaceutically acceptable salt dissolved in a pharmaceutically acceptable liquid, e.g., water or a pharmaceutically acceptable buffer.

Various technologies for administering compounds or compositions can be utilized in accordance with the present disclosure. For example, in some embodiments, provided compounds or compositions are administered orally. In some embodiments, provided compounds or compositions are administered intravenously.

In some embodiments, the composition is administered intravenously to the subject. In some embodiments, the composition is administered to a hepatocyte of the subject. In some embodiments, the composition is administered to the hepatocyte ex vivo. In some embodiments, the composition is administered to the hepatocyte in vivo.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in, e.g., “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included.

Aliphatic: As used herein, “aliphatic” means a straight-chain (i.e., unbranched) or branched, substituted or unsubstituted hydrocarbon chain that is completely saturated or that contains one or more units of unsaturation (but not aromatic), or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an aliphatic group, as defined herein, having one or more triple bonds.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, a genetically-engineered animal, and/or a clone.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, each monocyclic ring unit is aromatic. In some embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Complement component: As used herein, the terms “complement component” or “complement protein” is a molecule that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, H, and I, and properdin, with factor H being a negative regulator of the pathway. Components of the lectin pathway include, e.g., MBL2, MASP-1, and MASP-2. Complement components also include cell-bound receptors for soluble complement components. Such receptors include, e.g., C5a receptor (C5aR), C3a receptor (C3aR), Complement Receptor 1 (CR1), Complement Receptor 2 (CR2), Complement Receptor 3 (CR3), etc. It will be appreciated that the term “complement component” is not intended to include those molecules and molecular structures that serve as “triggers” for complement activation, e.g., antigen-antibody complexes, foreign structures found on microbial or artificial surfaces, etc.

Concurrent administration: As used herein, the term “concurrent administration” with respect to two or more agents, e.g., therapeutic agents, is administration performed using doses and time intervals such that the administered agents are present together within the body, e.g., at one or more sites of action in the body, over a time interval in non-negligible quantities. The time interval can be minutes (e.g., at least 1 minute, 1-30 minutes, 30-60 minutes), hours (e.g., at least 1 hour, 1-2 hours, 2-6 hours, 6-12 hours, 12-24 hours), days (e.g., at least 1 day, 1-2 days, 2-4 days, 4-7 days, etc.), weeks (e.g., at least 1, 2, or 3 weeks, etc.). Accordingly, the agents may, but need not be, administered together as part of a single composition. In addition, the agents may, but need not be, administered essentially simultaneously (e.g., within less than 5 minutes, or within less than 1 minute apart) or within a short time of one another (e.g., less than 1 hour, less than 30 minutes, less than 10 minutes, approximately 5 minutes apart). According to various embodiments of the disclosure, agents administered within such time intervals may be considered to be administered at substantially the same time. In certain embodiments of the disclosure, concurrently administered agents are present at effective concentrations within the body (e.g., in the blood and/or at a site of local complement activation) over the time interval. When administered concurrently, the effective concentration of each of the agents needed to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. The non-negligible concentration of an agent may be, for example, less than approximately 5% of the concentration that would be required to elicit a particular biological response, e.g., a desired biological response.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, each monocyclic ring unit is aromatic. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is boron, oxygen, sulfur, nitrogen, phosphorus, or silicon (including oxidized forms of nitrogen, sulfur, phosphorus, or silicon; charged forms of nitrogen (e.g., quaternized forms, forms as in iminium groups, etc.), phosphorus, sulfur, oxygen; etc.). In some embodiments, a heteroatom is silicon, phosphorus, oxygen, sulfur or nitrogen. In some embodiments, a heteroatom is silicon, oxygen, sulfur or nitrogen. In some embodiments, a heteroatom is oxygen, sulfur or nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Local administration: As used herein, the term “local administration” or “local delivery”, in reference to delivery of a composition or agent, refers to delivery that does not rely upon transport of the composition or agent to its intended target tissue or site via the vascular system. The composition or agent may be delivered directly to its intended target tissue or site, or in the vicinity thereof, e.g., in close proximity to the intended target tissue or site. For example, the composition may be delivered by injection or implantation of the composition or agent or by injection or implantation of a device containing the composition or agent. Following local administration in the vicinity of a target tissue or site, the composition or agent, or one or more components thereof, may diffuse to the intended target tissue or site. It will be understood that once having been locally delivered a fraction of a therapeutic agent (typically only a minor fraction of the administered dose) may enter the vascular system and be transported to another location, including back to its intended target tissue or site.

Local complement activation: As used herein, the term “local complement activation” refers to complement activation that occurs outside the vascular system.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, and a pharmaceutically acceptable salt is an alkali, alkaline earth metal, or ammonium (e.g., an ammonium salt of N(R)₃, wherein each R is optionally substituted C₁-C₆ alkyl) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups. In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations.

Protecting group: The term “protecting group,” as used herein, is well known in the art and includes those described in detail in Protecting Groups in Organic Synthesis, T. W. Greene and P. G. M. Wuts, 3P edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), 0-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Sequential administration: As used herein, the term “Sequential administration” of two or more agents refers to administration of two or more agents to a subject such that the agents are not present together in the subject's body, or at a relevant site of activity in the body, at greater than non-negligible concentrations. Administration of the agents may, but need not, alternate. Each agent may be administered multiple times.

Subject: As used herein, the term “subject” or “test subject” refers to any organism to which a provided compound or composition is administered in accordance with the present disclosure, e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition. In many instances, provided compounds or compositions are administered or delivered to human subjects.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual who is “suffering from” a disease, disorder, and/or condition has been diagnosed with and/or displays one or more symptoms of a disease, disorder, and/or condition.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Systemic: As used herein, the term “systemic” in reference to complement components, refers to complement proteins that are synthesized by liver hepatocytes and enter the bloodstream, or are synthesized by circulating macrophages or monocytes and secreted into the bloodstream.

Systemic complement activation: As used herein, the term “systemic complement activation” is complement activation that occurs in the blood, plasma, or serum and/or involves activation of systemic complement proteins at many locations throughout the body, affecting many body tissues, systems, or organs.

Systemic administration: As used herein, the term “systemic administration” and like terms are used herein consistently with their usage in the art to refer to administration of an agent such that the agent becomes widely distributed in the body in significant amounts and has a biological effect, e.g., its desired effect, in the blood and/or reaches its desired site of action via the vascular system. Typical systemic routes of administration include administration by (i) introducing the agent directly into the vascular system or (ii) subcutaneous, oral, pulmonary, or intramuscular administration wherein the agent is absorbed, enters the vascular system, and is carried to one or more desired site(s) of action via the blood.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or signs of the disease, disorder, and/or condition. In some embodiments, a therapeutically effective amount is administered in a single dose; in some embodiments, multiple unit doses are required to deliver a therapeutically effective amount.

Treating: As used herein, the term “treating” refers to providing treatment, i.e, providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of a complement-mediated condition, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition of the disclosure can be administered to a subject who has developed a complement-mediated disorder or is at increased risk of developing such a disorder relative to a member of the general population. A composition of the disclosure can be administered prophylactically, i.e., before development of any symptom or manifestation of the condition. Typically in this case the subject will be at risk of developing the condition.

As those skilled in the art will appreciate, methods and compositions described herein relating to provided compounds generally also apply to pharmaceutically acceptable salts of such compounds. Unless specified otherwise, solvates, stereoisomers, tautomers, salts of provided compounds are included.

### DETAILED DESCRIPTION OF CERTAIN EMBODIMENTS

Technologies of the present disclosure may be understood more readily by reference to the following detailed description of certain embodiments.

### I. Complement System

To facilitate understanding of the disclosure, and without intending to limit the provided technologies in any way, this section provides an overview of complement and its pathways of activation. Further details are found, e.g., in Kuby Immunology, 6th ed., 2006; Paul, W. E., Fundamental Immunology, Lippincott Williams & Wilkins; 6th ed., 2008; and Walport MJ., Complement. First of two parts. N Engl J Med., 344(14):1058-66, 2001.

Complement is an arm of the innate immune system that plays an important role in defending the body against infectious agents. The complement system comprises more than 30 serum and cellular proteins that are involved in three major pathways, known as the classical, alternative, and lectin pathways. The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 to form C3a and C3b. Binding of C3b to C3 convertase produces C5 convertase, which cleaves C5 into C5a and C5b. C3a, C4a, and C5a are anaphylotoxins and mediate multiple reactions in the acute inflammatory response. C3a and C5a are also chemotactic factors that attract immune system cells such as neutrophils. It will be understood that the names “C2a” and “C2b” used initially were subsequently reversed in the scientific literature.

The alternative pathway is initiated by and amplified at, e.g., microbial surfaces and various complex polysaccharides. In this pathway, hydrolysis of C3 to C3(H₂O), which occurs spontaneously at a low level, leads to binding of factor B, which is cleaved by factor D, generating a fluid phase C3 convertase that activates complement by cleaving C3 into C3a and C3b. C3b binds to targets such as cell surfaces and forms a complex with factor B, which is later cleaved by factor D, resulting in a C3 convertase. Surface-bound C3 convertases cleave and activate additional C3 molecules, resulting in rapid C3b deposition in close proximity to the site of activation and leading to formation of additional C3 convertase, which in turn generates additional C3b. This process results in a cycle of C3 cleavage and C3 convertase formation that significantly amplifies the response. Cleavage of C3 and binding of another molecule of C3b to the C3 convertase gives rise to a C5 convertase. C3 and C5 convertases of this pathway are regulated by cellular molecules CR1, DAF, MCP, CD59, and fH. The mode of action of these proteins involves either decay accelerating activity (i.e., ability to dissociate convertases), ability to serve as cofactors in the degradation of C3b or C4b by factor I, or both. Normally the presence of complement regulatory proteins on cell surfaces prevents significant complement activation from occurring thereon.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC). The MAC inserts itself into target cell membranes and causes cell lysis. Small amounts of MAC on the membrane of cells may have a variety of consequences other than cell death.

The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above.

Complement activity is regulated by various mammalian proteins referred to as complement control proteins (CCPs) or regulators of complement activation (RCA) proteins (U.S. Pat. No. 6,897,290). These proteins differ with respect to ligand specificity and mechanism(s) of complement inhibition. They may accelerate the normal decay of convertases and/or function as cofactors for factor I, to enzymatically cleave C3b and/or C4b into smaller fragments. CCPs are characterized by the presence of multiple (typically 4-56) homologous motifs known as short consensus repeats (SCR), complement control protein (CCP) modules, or SUSHI domains, about 50-70 amino acids in length that contain a conserved motif including four disulfide-bonded cysteines (two disulfide bonds), proline, tryptophan, and many hydrophobic residues. The CCP family includes complement receptor type 1 (CR1; C3b:C4b receptor), complement receptor type 2 (CR2), membrane cofactor protein (MCP; CD46), decay-accelerating factor (DAF), complement factor H (fH), and C4b-binding protein (C4bp). CD59 is a membrane-bound complement regulatory protein unrelated structurally to the CCPs. Complement regulatory proteins normally serve to limit complement activation that might otherwise occur on cells and tissues of the mammalian, e.g., human host. Thus, “self” cells are normally protected from the deleterious effects that would otherwise ensue were complement activation to proceed on these cells. Deficiencies or defects in complement regulatory protein(s) are involved in the pathogenesis of a variety of complement-mediated disorders, e.g., as discussed herein.

### II. Certain Provided Compounds and Composition

Among other things, the present disclosure provides compounds, e.g., those of formula I or pharmaceutically acceptable salts thereof, and compositions thereof:

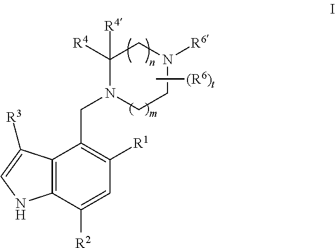

wherein each variable is independently as described herein.

In some embodiments, R¹ is hydrogen, halogen, hydroxy, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷. In some embodiments, R¹ is halogen, hydroxy, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷. In some embodiments, R¹ is hydrogen, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷. In some embodiments, R¹ is hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷. In some embodiments, R¹ is hydrogen, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷.

In some embodiments, R¹ is hydrogen. In some embodiments, R¹ is halogen. In some embodiments, R¹ is hydroxy. In some embodiments, R¹ is C₁-C₆ alkyl. In some embodiments, R¹ is C₂-C₆ alkenyl. In some embodiments, R¹ is C₃-C₆ cycloalkyl. In some embodiments, R¹ is cyclopropyl. In some embodiments, R¹ is halo C₁-C₆ alkyl. In some embodiments, R¹ is hydroxy C₁-C₆ alkyl. In some embodiments, R¹ is amino C₁-C₆ alkyl. In some embodiments, R¹ is C₁-C₆ alkoxy C₁-C₆ alkyl. In some embodiments, R¹ is C₁-C₆ alkoxy C₁-C₆ alkoxy. In some embodiments, R¹ is C₃-C₆ cycloalkyl C₁-C₆ alkoxy. In some embodiments, R¹ is halo C₁-C₆ alkoxy. In some embodiments, R¹ is —S(O)ₚC₁-C₆ alkyl. In some embodiments, R¹ is —SC₁-C₆ alkyl. In some embodiments, R¹ is —S(O)C₁-C₆ alkyl. In some embodiments, R¹ is —CH₂NHC(O)C₁-C₄ alkyl. In some embodiments, R¹ is —OCH₂C(O)R⁷.

In some embodiments, R¹ is C₁-C₆ alkoxy.

As those skilled in the art appreciate, a C₁ to C₆ (e.g., C₁-C₂, C₁-C₃, C₁-C₄, C₁-C₅, C₁-C₆, etc.) alkyl group, either independently or as part of another group (e.g., as in C₁-C₆ alkoxy, C₁-C₆ alkoxy C₁-C₆ alkyl, etc.), can independently be a C₁, C₂, C₃, C₄, C₅, or C₆ alkyl group. For example, in some embodiments, C₁-C₆ alkyl is methyl. In some embodiments, it is ethyl. In some embodiments, it is propyl. In some embodiments, it is n-propyl. In some embodiments, it is isopropyl. In some embodiments, it is butyl. In some embodiments, it is pentyl. In some embodiments, it is hexyl. In some embodiments, R¹ is C₁-C₆ alkoxy. In some embodiments, R¹ is —O—CH₃.

In some embodiments, p is 0. In some embodiments, p is 1. In some embodiments, p is 2.

In some embodiments, R¹ is —SC₁-C₆ alkyl. In some embodiments, R¹ is —S(O)C₁-C₆ alkyl. In some embodiments, R¹ is —S(O)₂C₁-C₆ alkyl.

In some embodiments, R² is hydrogen, C₁-C₆ alkyl, C₁-C₆ alkoxy, hydroxy C₁-C₆ alkyl, or halogen.

In some embodiments, R² is hydrogen.

In some embodiments, R² is C₁-C₆ alkyl. In some embodiments, R² is methyl. In some embodiments, R² is ethyl.

In some embodiments, R² is C₁-C₆ alkoxy. In some embodiments, R² is hydroxy C₁-C₆ alkyl. In some embodiments, R² is halogen. In some embodiments, R² is —F. In some embodiments, R² is —Cl. In some embodiments, R² is —Br. In some embodiments, R² is —I.

In some embodiments, R³ is hydrogen, halogen, cyano, C₁-C₄ alkyl, halo C₁-C₄ alkyl, —CH₂C(O)R⁷, phenyl or 5- or 6-membered heteroaryl having 1, 2 or 3 ring heteroatoms independently selected from nitrogen, oxygen and sulfur, wherein the phenyl or heteroaryl is optionally substituted with 1, or 2 C₁-C₄ alkyl groups, and wherein alkyl and haloalkyl are optionally substituted with 1 hydroxy group.

In some embodiments, R³ is hydrogen.

In some embodiments, R³ is halogen. In some embodiments, R³ is —F. In some embodiments, R³ is —Cl. In some embodiments, R³ is —Br. In some embodiments, R³ is —I. In some embodiments, R³ is cyano.

In some embodiments, R³ is C₁-C₄ alkyl. In some embodiments, R³ is C₁-C₄ alkyl optionally substituted with one hydroxy group. In some embodiments, R³ is C₁-C₄ alkyl substituted with one hydroxy group.

In some embodiments, R³ is halo C₁-C₄ alkyl. In some embodiments, R³ is halo C₁-C₄ alkyl optionally substituted with one hydroxy group. In some embodiments, R³ is halo C₁-C₄ alkyl substituted with one hydroxy group. In some embodiments, R³ is —CH₂C(O)R⁷.

In some embodiments, R³ is phenyl. In some embodiments, R³ is phenyl optionally substituted with 1 or 2 C₁-C₄ alkyl groups. In some embodiments, R³ is phenyl substituted with 1 or 2 C₁-C₄ alkyl groups.

In some embodiments, R³ is 5- or 6-membered heteroaryl having 1, 2 or 3 ring heteroatoms independently selected from nitrogen, oxygen and sulfur. In some embodiments, R³ is 5- or 6-membered heteroaryl having 1, 2 or 3 ring heteroatoms independently selected from nitrogen, oxygen and sulfur, wherein the heteroaryl is optionally substituted 1 or 2 C₁-C₄ alkyl groups. In some embodiments, R³ is 5- or 6-membered heteroaryl having 1, 2 or 3 ring heteroatoms independently selected from nitrogen, oxygen and sulfur, wherein the heteroaryl is substituted 1 or 2 C₁-C₄ alkyl groups.

In some embodiments, R⁴ is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl.

In some embodiments, R⁴ is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl.

In some embodiments, R⁴ is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is substituted with —C(O)R⁸ and further substituted with 0 or 1 substituent selected from halogen, C₁-C₄ alkyl, C₁-C₄ alkoxy, hydroxy C₁-C₄ alkyl, hydroxy, and cyanomethyl.

In some embodiments, R⁴ is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is substituted with —C(O)OH and further substituted with 0 or 1 substituent selected from halogen, C₁-C₄ alkyl, C₁-C₄ alkoxy, hydroxy C₁-C₄ alkyl, hydroxy, and cyanomethyl.

In some embodiments, R⁴ is phenyl. In some embodiments, R⁴ is phenyl substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl. In some embodiments, R⁴ is phenyl substituted with R⁵ only. In some embodiments, R⁴ is phenyl substituted with R⁵ and further substituted with one substituent selected from halogen, C₁-C₄ alkyl, C₁-C₄ alkoxy, hydroxy C₁-C₄ alkyl, hydroxy, and cyanomethyl. In some embodiments, R⁴ is phenyl substituted with —C(O)R⁸ and further substituted with 0 or 1 substituent selected from halogen, C₁-C₄ alkyl, C₁-C₄ alkoxy, hydroxy C₁-C₄ alkyl, hydroxy, and cyanomethyl. In some embodiments, R⁴ is phenyl substituted with —C(O)R⁸ only. In some embodiments, R⁴ is phenyl substituted with —C(O)R⁸ and further substituted with one substituent selected from halogen, C₁-C₄ alkyl, C₁-C₄ alkoxy, hydroxy C₁-C₄ alkyl, hydroxy, and cyanomethyl. In some embodiments, R⁴ is phenyl substituted with —COOH and further substituted with 0 or 1 substituent selected from halogen, C₁-C₄ alkyl, C₁-C₄ alkoxy, hydroxy C₁-C₄ alkyl, hydroxy, and cyanomethyl. In some embodiments, R⁴ is phenyl substituted with —COOH only. In some embodiments, R⁴ is phenyl substituted with —COOH and further substituted with one substituent selected from halogen, C₁-C₄ alkyl, C₁-C₄ alkoxy, hydroxy C₁-C₄ alkyl, hydroxy, and cyanomethyl.

In some embodiments, R⁴ is naphthyl. In some embodiments, R⁴ is naphthyl substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl. In some embodiments, R⁴ is naphthyl substituted with R⁵ only. In some embodiments, R⁴ is naphthyl substituted with R⁵ and further substituted with one substituent selected from halogen, C₁-C₄ alkyl, C₁-C₄ alkoxy, hydroxy C₁-C₄ alkyl, hydroxy, and cyanomethyl. In some embodiments, R⁴ is naphthyl substituted with —C(O)R⁸ and further substituted with 0 or 1 substituent selected from halogen, C₁-C₄ alkyl, C₁-C₄ alkoxy, hydroxy C₁-C₄ alkyl, hydroxy, and cyanomethyl. In some embodiments, R⁴ is naphthyl substituted with —C(O)R⁸ only. In some embodiments, R⁴ is naphthyl substituted with —C(O)R⁸ and further substituted with one substituent selected from halogen, C₁-C₄ alkyl, C₁-C₄ alkoxy, hydroxy C₁-C₄ alkyl, hydroxy, and cyanomethyl. In some embodiments, R⁴ is naphthyl substituted with —COOH and further substituted with 0 or 1 substituent selected from halogen, C₁-C₄ alkyl, C₁-C₄ alkoxy, hydroxy C₁-C₄ alkyl, hydroxy, and cyanomethyl. In some embodiments, R⁴ is naphthyl substituted with —COOH only. In some embodiments, R⁴ is naphthyl substituted with —COOH and further substituted with one substituent selected from halogen, C₁-C₄ alkyl, C₁-C₄ alkoxy, hydroxy C₁-C₄ alkyl, hydroxy, and cyanomethyl.

In some embodiments, R⁴ is tetrahydronaphthalenyl. In some embodiments, R⁴ is tetrahydronaphthalenyl substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl. In some embodiments, R⁴ is tetrahydronaphthalenyl substituted with R⁵ only. In some embodiments, R⁴ is tetrahydronaphthalenyl substituted with R⁵ and further substituted with one substituent selected from halogen, C₁-C₄ alkyl, C₁-C₄ alkoxy, hydroxy C₁-C₄ alkyl, hydroxy, and cyanomethyl. In some embodiments, R⁴ is tetrahydronaphthalenyl substituted with —C(O)R⁸ and further substituted with 0 or 1 substituent selected from halogen, C₁-C₄ alkyl, C₁-C₄ alkoxy, hydroxy C₁-C₄ alkyl, hydroxy, and cyanomethyl. In some embodiments, R⁴ is tetrahydronaphthalenyl substituted with —C(O)R⁸ only. In some embodiments, R⁴ is tetrahydronaphthalenyl substituted with —C(O)R⁸ and further substituted with one substituent selected from halogen, C₁-C₄ alkyl, C₁-C₄ alkoxy, hydroxy C₁-C₄ alkyl, hydroxy, and cyanomethyl. In some embodiments, R⁴ is tetrahydronaphthalenyl substituted with —COOH and further substituted with 0 or 1 substituent selected from halogen, C₁-C₄ alkyl, C₁-C₄ alkoxy, hydroxy C₁-C₄ alkyl, hydroxy, and cyanomethyl. In some embodiments, R⁴ is tetrahydronaphthalenyl substituted with —COOH only. In some embodiments, R⁴ is tetrahydronaphthalenyl substituted with —COOH and further substituted with one substituent selected from halogen, C₁-C₄ alkyl, C₁-C₄ alkoxy, hydroxy C₁-C₄ alkyl, hydroxy, and cyanomethyl.

In some embodiments, R⁴ is 5- or 6-membered heteroaryl having 1, 2 or 3 ring heteroatoms independently selected from nitrogen, oxygen and sulfur. In some embodiments, R⁴ is 5- or 6-membered heteroaryl having 1, 2 or 3 ring heteroatoms independently selected from nitrogen, oxygen and sulfur, wherein the heteroaryl is substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl. In some embodiments, the heteroaryl is substituted with R⁵ only. In some embodiments, the heteroaryl is substituted with R⁵ and further substituted with one substituent selected from halogen, C₁-C₄ alkyl, C₁-C₄ alkoxy, hydroxy C₁-C₄ alkyl, hydroxy, and cyanomethyl. In some embodiments, R⁴ is 5- or 6-membered heteroaryl having 1, 2 or 3 ring heteroatoms independently selected from nitrogen, oxygen and sulfur, wherein the heteroaryl substituted with —C(O)R⁸ and further substituted with 0 or 1 substituent selected from halogen, C₁-C₄ alkyl, C₁-C₄ alkoxy, hydroxy C₁-C₄ alkyl, hydroxy, and cyanomethyl. In some embodiments, the heteroaryl is substituted with —C(O)R⁸ only. In some embodiments, the heteroaryl substituted with —C(O)R⁸ and further substituted with one substituent selected from halogen, C₁-C₄ alkyl, C₁-C₄ alkoxy, hydroxy C₁-C₄ alkyl, hydroxy, and cyanomethyl. In some embodiments, R⁴ is 5- or 6-membered heteroaryl having 1, 2 or 3 ring heteroatoms independently selected from nitrogen, oxygen and sulfur, wherein the heteroaryl substituted with —COOH and further substituted with 0 or 1 substituent selected from halogen, C₁-C₄ alkyl, C₁-C₄ alkoxy, hydroxy C₁-C₄ alkyl, hydroxy, and cyanomethyl. In some embodiments, the heteroaryl is substituted with —COOH only. In some embodiments, the heteroaryl substituted with —COOH and further substituted with one substituent selected from halogen, C₁-C₄ alkyl, C₁-C₄ alkoxy, hydroxy C₁-C₄ alkyl, hydroxy, and cyanomethyl. In some embodiments, the heteroaryl of R⁴ is 5-membered. In some embodiments, it is 6-membered. In some embodiments, it has 1 heteroatom. In some embodiments, it has 1 heteroatom which is nitrogen. In some embodiments, it has 1 heteroatom which is oxygen. In some embodiments, it has 1 heteroatom which is sulfur. In some embodiments, it has 2 or 3 ring heteroatoms. In some embodiments, it has 2 or 3 ring heteroatoms at least one of which is nitrogen. In some embodiments, it is

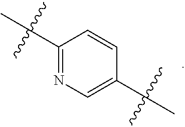 [SMILES: *c1ccc(*)nc1]

In some embodiments, it is bonded to R⁵, e.g., —COOH, at the o-positive relative to the nitrogen atom.

In some embodiments, the further substituent on R⁴ in addition to R⁵ is halogen. In some embodiments, it is —F. In some embodiments, it is —Cl. In some embodiments, it is —Br. In some embodiments, it is —I. In some embodiments, it is C₁-C₄ alkyl. In some embodiments, it is C₁-C₄ alkoxy. In some embodiments, it is hydroxy C₁-C₄ alkyl. In some embodiments, it is hydroxy. In some embodiments, it is cyanomethyl.

In some embodiments, R^{4′} is hydrogen. In some embodiments, R^{4′} is C₁-C₄ alkyl. In some embodiments, R^{4′} is hydroxy C₁-C₄ alkyl.

In some embodiments, R⁵ is —C(O)R⁸, —CH₂C(O)R⁸, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R′)₂ or 5-membered heteroaryl having 1-4 ring nitrogen atoms and 0 or 1 ring oxygen or sulfur atom.

In some embodiments, R⁵ is —COOH or a bioisostere thereof.

In some embodiments, R⁵ is —C(O)R⁸. In some embodiments, R⁵ is —C(O)OH. In some embodiments, R⁵ is —C(O)R⁸, wherein R⁸ is C₁-C₄ alkoxy.

In some embodiments, R⁵ is —CH₂C(O)R⁸. In some embodiments, R⁵ is —CH₂C(O)OH. In some embodiments, R⁵ is —CH₂C(O)R⁸, wherein R⁸ is C₁-C₄ alkoxy.

In some embodiments, R⁵ is R⁹. In some embodiments, R⁵ is —C(O)NHSO₂C₁-C₄alkyl. In some embodiments, R⁵ is —C(O)NHSO₂CH₃. In some embodiments, R⁵ is —C(O)NHSO₂(cyclopropyl). In some embodiments, R⁵ is —SO₂NHC(O)C₁-C₄alkyl. In some embodiments, R⁵ is —SO₂N(H)ₚ(C₁-C₄alkyl)₂-p. In some embodiments, R⁵ is —SO₂N(C₁-C₄alkyl)₂. In some embodiments, R⁵ is —SO₂NH(C₁-C₄alkyl). In some embodiments, R⁵ is —SO₂NH₂. In some embodiments, R⁵ is —SO(NH)C₁-C₄alkyl. In some embodiments, R⁵ is —SO₂C₁-C₄alkyl. In some embodiments, R⁵ is cyano. In some embodiments, R⁵ is halogen. In some embodiments, R⁵ is —F. In some embodiments, R⁵ is —Cl. In some embodiments, R⁵ is —Br. In some embodiments, R⁵ is —I. In some embodiments, R⁵ is hydroxy C₁-C₄ alkyl.

In some embodiments, R⁵ is —B(R′)₂. In some embodiments, R⁵ is —B(R′)₂, wherein each R⁸ is independently hydroxy or C₁-C₄ alkoxy. In some embodiments, R⁵ is —B(OH)₂. In some embodiments, R⁵ is —B(R′)₂, wherein each R⁸ is independently C₁-C₄ alkoxy.

In some embodiments, R⁵ is 5-membered heteroaryl having 1-4 ring nitrogen atoms and 0 or 1 ring oxygen or sulfur atom. In some embodiments, R⁵ is 5-membered heteroaryl having 1-4 ring nitrogen atoms.

In some embodiments, each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁—C₆ alkyl or C₁-C₆ alkoxy.

In some embodiments, R⁶ is hydrogen. In some embodiments, R⁶ is not hydrogen. In some embodiments, R⁶ is halogen. In some embodiments, R⁶ is —F. In some embodiments, R⁶ is —Cl. In some embodiments, R⁶ is —Br. In some embodiments, R⁶ is —I. In some embodiments, R⁶ is hydroxy. In some embodiments, R⁶ is amino. In some embodiments, R⁶ is mono- and di-C₁-C₆ alkylamino. In some embodiments, R⁶ is C₁-C₆ alkyl. In some embodiments, R⁶ is halo C₁-C₆ alkyl. In some embodiments, R⁶ is hydroxy C₁-C₆ alkyl. In some embodiments, R⁶ is cyano C₁-C₆ alkyl. In some embodiments, R⁶ is C₁-C₆ alkoxy.

In some embodiments, two R⁶ on a carbon atom are taken together to form ═O. In some embodiments, ═O is on a carbon at the o-position relative to the carbon to which R⁴ and R^{4′} are attached. In some embodiments, ═O is on a carbon at the p-position relative to the carbon to which R⁴ and R^{4′} are attached. In some embodiments, two R⁶ on a carbon atom are taken together with the carbon atom to form a 3-membered monocyclic saturated ring having 0-1 ring atoms independently selected from nitrogen, sulfur and oxygen. In some embodiments, a formed ring is

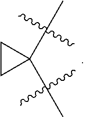 [SMILES: *C1(*)CC1]

In some embodiments, a formed ring is

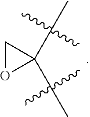 [SMILES: *C1(*)CO1]

In some embodiments, two R⁶ on a carbon atom are taken together with the carbon atom to form a 3-membered monocyclic saturated ring having a nitrogen ring atom. In some embodiments, two R⁶ on a carbon atom are taken together with the carbon atom to form a 3-membered monocyclic saturated ring having a sulfur ring atom.

In some embodiments, two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered. In some embodiments, a formed ring is saturated. In some embodiments, a formed ring is partially unsaturated. In some embodiments, a formed ring has no heteroatom ring atom. In some embodiments, a formed ring has 1-3 ring atoms independently selected from nitrogen, sulfur and oxygen. In some embodiments, one ring atom is a heteroatom. In some embodiments, two ring atoms are independently a heteroatom. In some embodiments, three ring atoms are independently a heteroatom. In some embodiments, a ring atom is nitrogen. In some embodiments, a ring atom is sulfur. In some embodiments, a ring atom is oxygen. In some embodiments, a formed ring is substituted. In some embodiments, a formed ring is unsubstituted. In some embodiments, a substituent is halogen. In some embodiments, a substituent is C₁-C₄ alkyl. In some embodiments, a substituent is halo C₁-C₄ alkyl. In some embodiments, a substituent is C₁-C₄ alkoxy. In some embodiments, a substituent is hydroxy. In some embodiments, a substituent is carbonyl.

In various embodiments, “carbonyl” is utilized to refer to a substituent (e.g., when a group is substituted with carbonyl), and it means a carbon is substituted with ═O to form a carbonyl group.

In some embodiments, t is 0. In some embodiments, t is 1. In some embodiments, t is 3. In some embodiments, t is 4. In some embodiments, t is 1, 2, 3 or 4, and each R⁶ is not hydrogen. In some embodiments, t is 1, 2, 3, or 4, and each R⁶ is independently halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy.

In some embodiments, R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic, C₃-C₆ cycloaliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl.

In some embodiments, R^{6′} is hydrogen. In some embodiments, R^{6′} is not hydrogen.

In some embodiments, R^{6′} is C₁-C₆ aliphatic optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl.

In some embodiments, R^{6′} is C₁₋₆ aliphatic. In some embodiments, R^{6′} is C₁-C₆ alkyl optionally substituted as described herein. In some embodiments, R^{6′} is C₁-C₆ alkyl. In some embodiments, R^{6′} is C₁. In some embodiments, R^{6′} is C₂. In some embodiments, R^{6′} is C₃. In some embodiments, R^{6′} is C₄. In some embodiments, R^{6′} is C₅. In some embodiments, R^{6′} is C₆. In some embodiments, R^{6′} is C₁-C₅. In some embodiments, R^{6′} is C₁-C₄. In some embodiments, R^{6′} is C₁-C₃. In some embodiments, R^{6′} is C₁-C₂. In some embodiments, R^{6′} is C₂-C₆. In some embodiments, R^{6′} is C₂-C₅. In some embodiments, R^{6′} is C₂-C₄. In some embodiments, R^{6′} is C₂-C₃. In some embodiments, R^{6′} is C₃-C₆. In some embodiments, R^{6′} is methyl. In some embodiments, R^{6′} is ethyl. In some embodiments, R^{6′} is n-propyl. In some embodiments, R^{6′} is isopropyl. In some embodiments, R^{6′} is —CH₂-cyclopropyl. In some embodiments, R^{6′} is —CH₂-cyclobutyl. In some embodiments, R^{6′} is —CH₂-cyclopentyl. In some embodiments, R^{6′} is 2-methylpropyl. In some embodiments, R^{6′} is 1-methylpropyl. In some embodiments, R^{6′} is n-butyl. In some embodiments, R^{6′} is —CH₂CH(CH₃)CH₂CH₃. In some embodiments, R^{6′} is —CH₂CH₂CH(CH₃)₂.

In some embodiments, R^{6′} is C₃₋₆(C₃, C₄, C₅ or C₆) cycloaliphatic optionally substituted as described herein. In some embodiments, R^{6′} is C₃-C₆ (C₃, C₄, C₅ or C₆) cycloalkyl optionally substituted as described herein. In some embodiments, R^{6′} is C₃₋₆ (C₃, C₄, C₅ or C₆) cycloaliphatic. In some embodiments, R^{6′} is C₃-C₆ (C₃, C₄, C₅ or C₆) cycloalkyl. In some embodiments, cycloalkyl is cyclopropyl. In some embodiments, it is cyclobutyl. In some embodiments, it is cyclopentyl. In some embodiments, it is cyclohexyl.

In some embodiments, R^{6′} is C₂-C₆ alkenyl optionally substituted as described herein. In some embodiments, R^{6′} is C₂-C₆ alkenyl. In some embodiments, an alkenyl is a terminal alkenyl group. In some embodiments, R^{6′} is —CH₂CH═CH₂.

In some embodiments, R^{6′} is C₂-C₆ alkynyl optionally substituted as described herein. In some embodiments, R^{6′} is C₂-C₆ alkynyl. In some embodiments, an alkynyl is a terminal alkynyl group. In some embodiments, R^{6′} is —CH₂C≡CH.

In some embodiments, R^{6′} is —CH₂-(hydroxy C₁-C₄ alkyl). In some embodiments, R^{6′} is —CH₂CH₂OH.

In some embodiments, a group (e.g., C₁₋₆ aliphatic, C₃-C₆ cycloaliphatic, phenyl, etc.) of R^{6′} is substituted as described herein. In some embodiments, it is substituted with halogen (e.g., —F, —Cl, —Br or —I). In some embodiments, it is substituted with —F. For example, in some embodiments, R^{6′} is C₁₋₆ aliphatic substituted with one or more —F. In some embodiments, R^{6′} is C₁₋₆ alkyl substituted with one or more —F. In some embodiments, R^{6′} is —(CH₂)₂—F. In some embodiments, R^{6′} is —(CH₂)₃—F. In some embodiments, R^{6′} is —CH₂—CHF₂. In some embodiments, R^{6′} is —CH(CH₃)—CHF₂. In some embodiments, R^{6′} is —CH₂—CF₃. In some embodiments, R^{6′} is —CH₂—CH₂—CHF₂. In some embodiments, R^{6′} is —CH₂—CH₂—CF₃. In some embodiments, R^{6′} is —CH₂—CF₂—CH₃. In some embodiments, R^{6′} is —CH(CH₃)—CH₂—CF₃. In some embodiments, R^{6′} is C₃-C₆ substituted one or more —F. In some embodiments, R^{6′} is cyclopropyl substituted with —F.

In some embodiments, a group (e.g., C₁₋₆ aliphatic, C₃-C₆ cycloaliphatic, phenyl, etc.) of R^{6′} is substituted as described herein. In some embodiments, it is substituted with C₁-C₄ (e.g., C₁, C₂, C₃ or C₄) alkoxy. In some embodiments, it is substituted with CH₃O—. In some embodiments, R^{6′} is —CH₂CH₂OCH₃.

In some embodiments, R^{6′} is R¹⁰. In some embodiments, R^{6′} is 3-6 membered heterocyclyl optionally substituted as described herein. In some embodiments, R^{6′} is 3-membered heterocyclyl optionally substituted as described herein. In some embodiments, R^{6′} is 4-membered heterocyclyl optionally substituted as described herein. In some embodiments, R^{6′} is 5-membered heterocyclyl optionally substituted as described herein. In some embodiments, R^{6′} is 6-membered heterocyclyl optionally substituted as described herein. In some embodiments, a heterocyclyl group is substituted. In some embodiments, it is unsubstituted. In some embodiments, a heterocyclyl group has a ring oxygen atom. In some embodiments, R^{6′} is

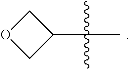 [SMILES: *C1COC1]

In some embodiments, R^{6′} is 5-6 membered heteroaryl optionally substituted as described herein. In some embodiments, R^{6′} is 5-membered heteroaryl optionally substituted as described herein. In some embodiments, R^{6′} is 6-membered heteroaryl optionally substituted as described herein. In some embodiments, a heteroaryl group is substituted. In some embodiments, a heteroaryl group is unsubstituted. In some embodiments, R^{6′} is

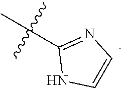 [SMILES: *c1ncc[nH]1]

In some embodiments, R^{6′} is

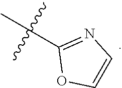 [SMILES: *c1ncco1]

In some embodiments, R^{6′} is

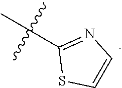 [SMILES: *c1nccs1]

In some embodiments, R^{6′} is

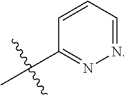 [SMILES: *c1cccnn1]

In some embodiments, R^{6′} is

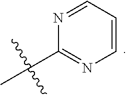 [SMILES: *c1ncccn1]

In some embodiments, the atom of R¹⁰ that forms a bond with the nitrogen to which R¹⁰ is attached is a carbon atom.

In some embodiments, R^{6′} is —CH₂—C₃-C₆ cycloaliphatic wherein the cycloaliphatic group is optionally substituted as described herein. In some embodiments, a cycloaliphatic group is a cycloalkyl group. In some embodiments, R^{6′} is —CH₂—C₃-C₆ cycloalkyl wherein the cycloaliphatic group is optionally substituted as described herein. In some embodiments, R^{6′} is —CH₂—C₃-C₆ cycloaliphatic. In some embodiments, R^{6′} is —CH₂—C₃-C₆ cycloalkyl. In some embodiments, a cycloalkyl is cyclopropyl. In some embodiments, a cycloalkyl is cyclobutyl. In some embodiments, a cycloalkyl is cyclopentyl. In some embodiments, a cycloalkyl is cyclohexyl.

In some embodiments, R^{6′} is —CH₂—R¹⁰, wherein R¹⁰ is as described herein.

In some embodiments, R^{6′} is —CH₂-(hydroxy C₁-C₄ alkyl). In some embodiments, R^{6′} is —CH₂CH₂OH.

In some embodiments, R^{6′} is phenyl optionally substituted as described herein. In some embodiments, a phenyl group is substituted. In some embodiments, a phenyl group is substituted with one or more halogen. In some embodiments, a phenyl group is unsubstituted. In some embodiments, R^{6′} is phenyl.

In some embodiments, R^{6′} is —C(O)—C₁-C₆ aliphatic, wherein the C₁-C₆ aliphatic is as described herein. In some embodiments, R^{6′} is —C(O)—C₁-C₆ aliphatic, wherein the C₁-C₆ aliphatic is optionally substituted as described herein. In some embodiments, R^{6′} is —C(O)—C₁-C₆ alkyl. In some embodiments, R^{6′} is —C(O)—CH₃.

In some embodiments, R^{6′} is —SO₂—C₁-C₆ aliphatic, wherein the C₁-C₆ aliphatic is as described herein. In some embodiments, R^{6′} is —SO₂—C₁-C₆ aliphatic, wherein the C₁-C₆ aliphatic is optionally substituted as described herein. In some embodiments, R^{6′} is —SO₂—C₁-C₆ alkyl. In some embodiments, R^{6′} is —SO₂—CH₃.

In some embodiments, R^{6′} is —CH₂-phenyl, wherein the phenyl group is optionally substituted as described herein. In some embodiments, a phenyl group is substituted. In some embodiments, a phenyl group is substituted with one or more halogen. In some embodiments, a phenyl group is unsubstituted. In some embodiments, R^{6′} is —CH₂-phenyl.

In some embodiments, R^{6′} is —CH₂-(amino C₁-C₄ alkyl).

In some embodiments, R^{6′} is —CH₂-(mono- and di-C₁-C₄ alkylamino C₁-C₄ alkyl).

In some embodiments, R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered.

In some embodiments, R⁷ is hydroxy. In some embodiments, R⁷ is C₁-C₄ alkoxy. In some embodiments, R⁷ is amino. In some embodiments, R⁷ is mono-C₁-C₄ alkylamino. In some embodiments, R⁷ is di-C₁-C₄ alkylamino.

In some embodiments, each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or R⁸ is mono- and di-C₁-C₄ alkylamino which is optionally substituted with one or more substituents independently selected from halogen, hydroxy and C₁-C₄ alkyl.

In some embodiments, R⁸—OH. In some embodiments, R⁸ is C₁₋₄ alkoxy. In some embodiments, R⁸ is methoxy. In some embodiments, R⁸ is ethoxy. In some embodiments, R⁸ is amino. In some embodiments, R⁸ is 5-, 6- or 7-membered saturated heterocyclyl having 1, 2, or 3 ring heteroatoms independently selected from nitrogen, oxygen and sulfur. In some embodiments, R⁸ is mono-C₁-C₄ alkylamino which is optionally substituted with one or more substituents independently selected from halogen, hydroxy and C₁-C₄ alkyl. In some embodiments, R⁸ is mono-C₁-C₄ alkylamino. In some embodiments, R⁸ is di-C₁-C₄ alkylamino which is optionally substituted with one or more substituents independently selected from halogen, hydroxy and C₁-C₄ alkyl. In some embodiments, R⁸ is di-C₁-C₄ alkylamino.

In some embodiments, R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups. In some embodiments, R⁹ is a 5-membered heteroaryl having 1 to 4 ring nitrogen atoms and 0 ring oxygen or sulfur atom, which heteroaryl is optionally substituted with 0 to 2 C₁-C₄ alkyl groups. In some embodiments, a heteroaryl group is substituted. In some embodiments, a heteroaryl group is unsubstituted.

In some embodiments, R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur. In some embodiments, R¹⁰ is 3-, 4-, 5- or 6-membered heterocyclyl optionally substituted as described herein. In some embodiments, R¹⁰ is 5- or 6-membered heteroaryl optionally substituted as described herein. In some embodiments, a heterocyclyl group is substituted. In some embodiments, a heterocyclyl group is unsubstituted. In some embodiments, a heteroaryl group is substituted. In some embodiments, a heteroaryl group is unsubstituted. In some embodiments, at least one ring atom of heterocyclyl is nitrogen. In some embodiments, at least one ring atom of heteroaryl is nitrogen. In some embodiments, each heteroatom ring atom of heterocyclyl is nitrogen. In some embodiments, each heteroatom ring atom of heteroaryl is nitrogen.

In some embodiments, provided compounds comprise enriched levels of one or more isotopes. For example, in some embodiments, provided compounds comprise enriched levels of deuterium (D). In some embodiments, R^{6′} is —CD₃. Various technologies are available for incorporating enriched levels of various isotopes and can be utilized in accordance with the present disclosure. In some embodiments, a compound has an isotopic purity of about 5%-100% (e.g., about 5%, 10%, 20%, 25%, 30%, 40%, 50%, 60%, 70%, 75%, 80%, 85%, 90%, 95%, 96%, 97%, 98%, 99%, 99.5%, 99.9% or more, or about 5%-100%, 10%-100%, 20%-100%, 30%-100%, 50%-100%, 80%-100%, 90-100%, 95%-100%, 96%-100%, 97%-100%, 98%-100%, 99%-100%, 95-99%, 95%-99.5%, 95%-99.9%, etc). For example, in some embodiments, about 5%-100% (e.g., about 5%, 10%, 20%, 25%, 30%, 40%, 50%, 60%, 70%, 75%, 80%, 85%, 90%, 95%, 96%, 97%, 98%, 99%, 99.5%, 99.9% or more, or about 5%-100%, 10%-100%, 20%-100%, 30%-100%, 50%-100%, 80%-100%, 90-100%, 95%-100%, 96%-100%, 97%-100%, 98%-100%, 99%-100%, 95-99%, 95%-99.5%, 95%-99.9%, etc) of all molecules of a compound wherein R^{6′} is —CH₃ has R^{6′} being —CD₃.

In some embodiments, m is 0. In some embodiments, m is 1. In some embodiments, m is 2. In some embodiments, n is 1. In some embodiments, n is 2. In some embodiments, m is 1 and n is 1. In some embodiments, t is 0. In some embodiments, t is 1. In some embodiments, t is 2. In some embodiments, t is 3. In some embodiments, t is 4. In some embodiments, a provided compound has the structure of:

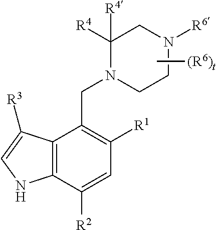

or a pharmaceutically acceptable salt thereof, wherein each variable is independently as described herein. In some embodiments, a provided compound has the structure of:

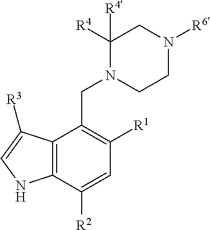

or a pharmaceutically acceptable salt thereof, wherein each variable is independently as described herein. In some embodiments, a provided compound has the structure of:

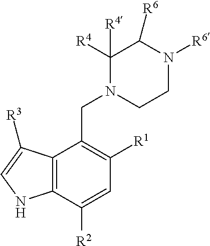

or a pharmaceutically acceptable salt thereof, wherein each variable is independently as described herein. In some embodiments, a provided compound has the structure of:

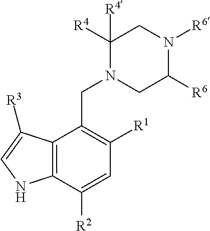

or a pharmaceutically acceptable salt thereof, wherein each variable is independently as described herein. In some embodiments, R⁶ and R^{6′} are taken together with their intervening atoms to form a ring as described herein. In some embodiments, a provided compound has the structure of:

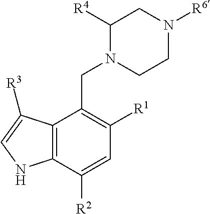

or a pharmaceutically acceptable salt thereof, wherein each variable is independently as described herein.

In some embodiments, a compound is selected from Table C-1 below, or a pharmaceutically acceptable salt thereof. In some embodiments, a composition comprises or delivers a compound selected from C-1 below, or a pharmaceutically acceptable salt thereof. In some embodiments, a compound is selected from Table E-1, or a pharmaceutically acceptable salt thereof. In some embodiments, a composition comprises or delivers a compound selected from E-1, or a pharmaceutically acceptable salt thereof.

TABLE C-1
Certain compounds.

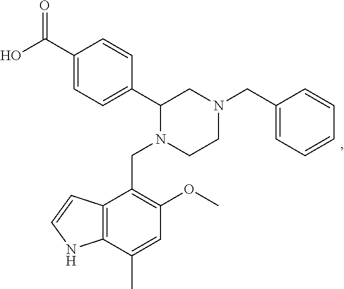 [SMILES: COc1cc(C)c2[nH]ccc2c1CN1CCN(Cc2ccccc2)CC1c1ccc(C(=O)O)cc1]

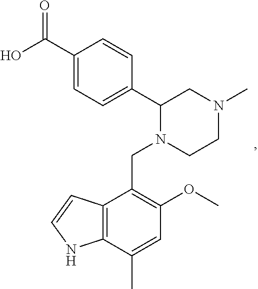 [SMILES: COc1cc(C)c2[nH]ccc2c1CN1CCN(C)CC1c1ccc(C(=O)O)cc1]

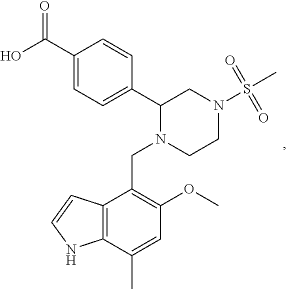 [SMILES: COc1cc(C)c2[nH]ccc2c1CN1CCN(S(C)(=O)=O)CC1c1ccc(C(=O)O)cc1]

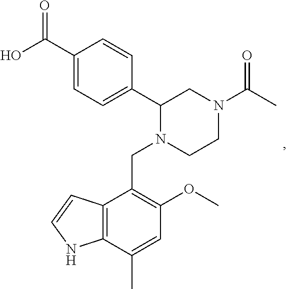 [SMILES: COc1cc(C)c2[nH]ccc2c1CN1CCN(C(C)=O)CC1c1ccc(C(=O)O)cc1]

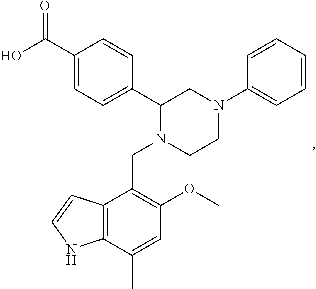 [SMILES: COc1cc(C)c2[nH]ccc2c1CN1CCN(c2ccccc2)CC1c1ccc(C(=O)O)cc1]

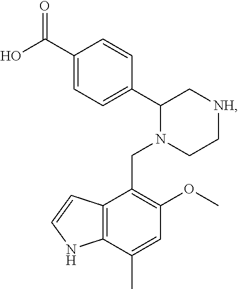 [SMILES: COc1cc(C)c2[nH]ccc2c1CN1CCNCC1c1ccc(C(=O)O)cc1]

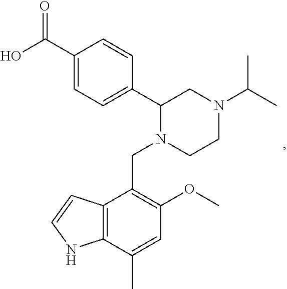 [SMILES: COc1cc(C)c2[nH]ccc2c1CN1CCN(C(C)C)CC1c1ccc(C(=O)O)cc1]

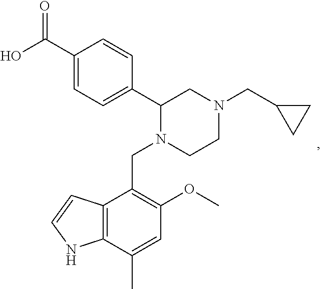 [SMILES: COc1cc(C)c2[nH]ccc2c1CN1CCN(CC2CC2)CC1c1ccc(C(=O)O)cc1]

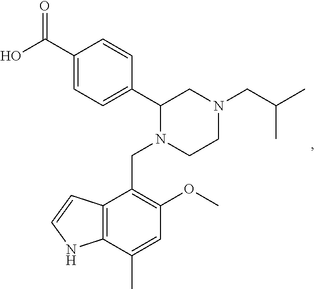 [SMILES: COc1cc(C)c2[nH]ccc2c1CN1CCN(CC(C)C)CC1c1ccc(C(=O)O)cc1]

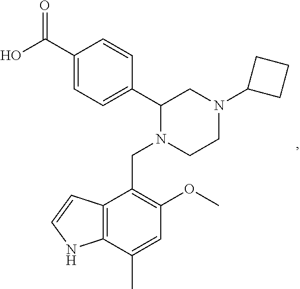 [SMILES: COc1cc(C)c2[nH]ccc2c1CN1CCN(C2CCC2)CC1c1ccc(C(=O)O)cc1]

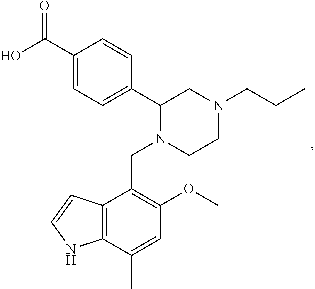 [SMILES: CCCN1CCN(Cc2c(OC)cc(C)c3[nH]ccc23)C(c2ccc(C(=O)O)cc2)C1]

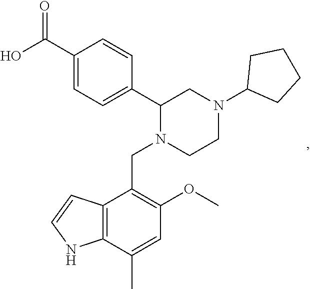 [SMILES: COc1cc(C)c2[nH]ccc2c1CN1CCN(C2CCCC2)CC1c1ccc(C(=O)O)cc1]

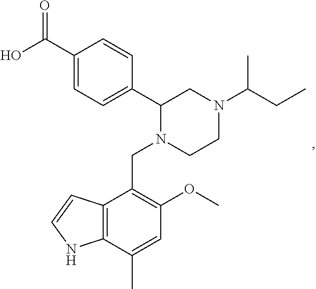 [SMILES: CCC(C)N1CCN(Cc2c(OC)cc(C)c3[nH]ccc23)C(c2ccc(C(=O)O)cc2)C1]

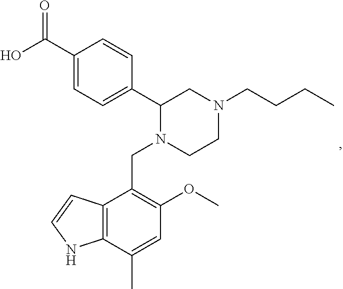 [SMILES: CCCCN1CCN(Cc2c(OC)cc(C)c3[nH]ccc23)C(c2ccc(C(=O)O)cc2)C1]

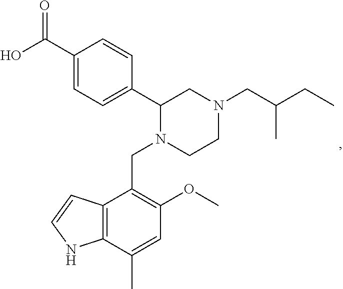 [SMILES: CCC(C)CN1CCN(Cc2c(OC)cc(C)c3[nH]ccc23)C(c2ccc(C(=O)O)cc2)C1]

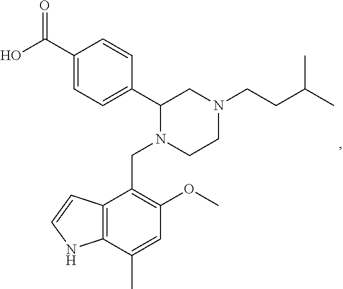 [SMILES: COc1cc(C)c2[nH]ccc2c1CN1CCN(CCC(C)C)CC1c1ccc(C(=O)O)cc1]

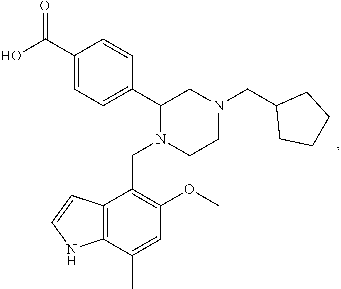 [SMILES: COc1cc(C)c2[nH]ccc2c1CN1CCN(CC2CCCC2)CC1c1ccc(C(=O)O)cc1]

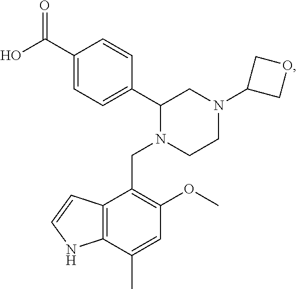 [SMILES: COc1cc(C)c2[nH]ccc2c1CN1CCN(C2COC2)CC1c1ccc(C(=O)O)cc1]

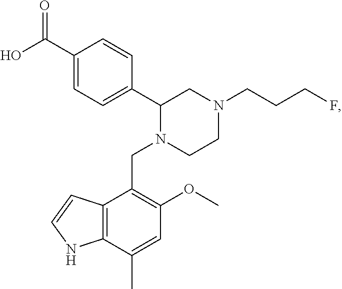 [SMILES: COc1cc(C)c2[nH]ccc2c1CN1CCN(CCCF)CC1c1ccc(C(=O)O)cc1]

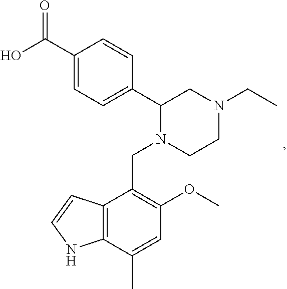 [SMILES: CCN1CCN(Cc2c(OC)cc(C)c3[nH]ccc23)C(c2ccc(C(=O)O)cc2)C1]

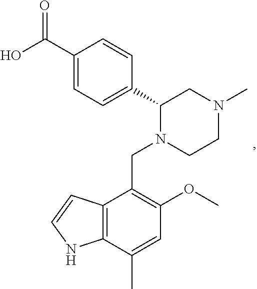 [SMILES: COc1cc(C)c2[nH]ccc2c1CN1CCN(C)C[C@H]1c1ccc(C(=O)O)cc1]

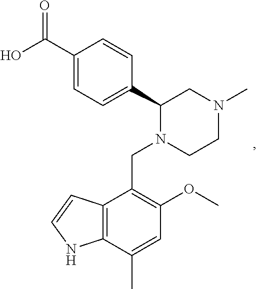 [SMILES: COc1cc(C)c2[nH]ccc2c1CN1CCN(C)C[C@@H]1c1ccc(C(=O)O)cc1]

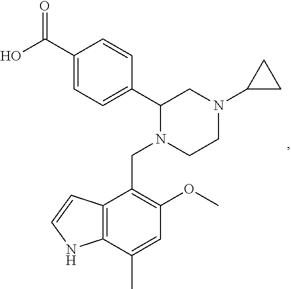 [SMILES: COc1cc(C)c2[nH]ccc2c1CN1CCN(C2CC2)CC1c1ccc(C(=O)O)cc1]

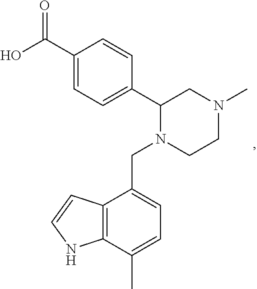 [SMILES: Cc1ccc(CN2CCN(C)CC2c2ccc(C(=O)O)cc2)c2cc[nH]c12]

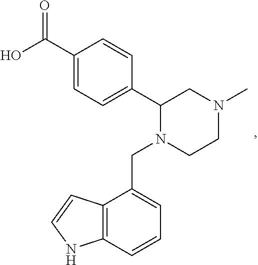 [SMILES: CN1CCN(Cc2cccc3[nH]ccc23)C(c2ccc(C(=O)O)cc2)C1]

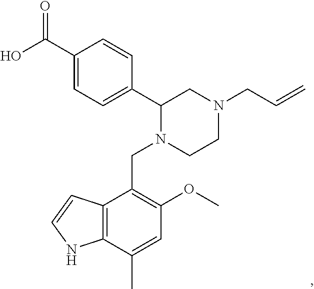 [SMILES: C=CCN1CCN(Cc2c(OC)cc(C)c3[nH]ccc23)C(c2ccc(C(=O)O)cc2)C1]

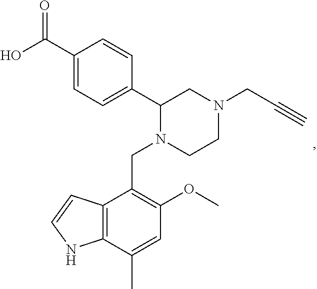 [SMILES: C#CCN1CCN(Cc2c(OC)cc(C)c3[nH]ccc23)C(c2ccc(C(=O)O)cc2)C1]

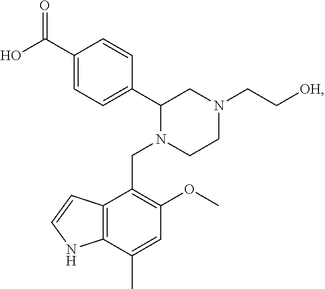 [SMILES: COc1cc(C)c2[nH]ccc2c1CN1CCN(CCO)CC1c1ccc(C(=O)O)cc1]

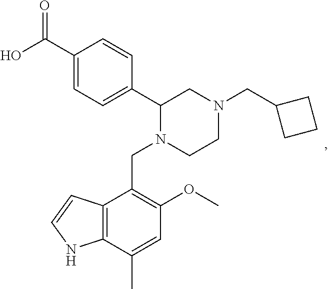 [SMILES: COc1cc(C)c2[nH]ccc2c1CN1CCN(CC2CCC2)CC1c1ccc(C(=O)O)cc1]

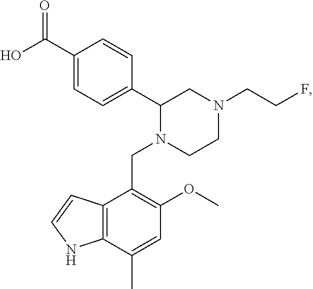 [SMILES: COc1cc(C)c2[nH]ccc2c1CN1CCN(CCF)CC1c1ccc(C(=O)O)cc1]

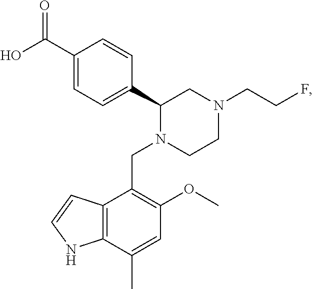 [SMILES: COc1cc(C)c2[nH]ccc2c1CN1CCN(CCF)C[C@@H]1c1ccc(C(=O)O)cc1]

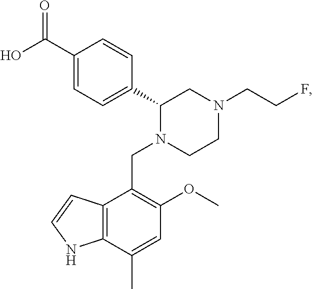 [SMILES: COc1cc(C)c2[nH]ccc2c1CN1CCN(CCF)C[C@H]1c1ccc(C(=O)O)cc1]

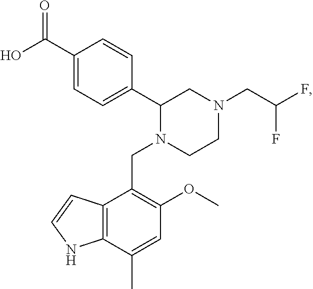 [SMILES: COc1cc(C)c2[nH]ccc2c1CN1CCN(CC(F)F)CC1c1ccc(C(=O)O)cc1]

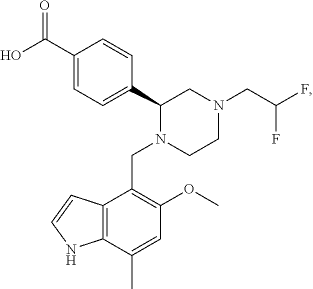 [SMILES: COc1cc(C)c2[nH]ccc2c1CN1CCN(CC(F)F)C[C@@H]1c1ccc(C(=O)O)cc1]

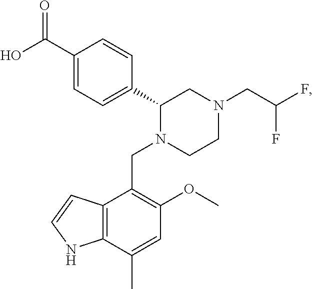 [SMILES: COc1cc(C)c2[nH]ccc2c1CN1CCN(CC(F)F)C[C@H]1c1ccc(C(=O)O)cc1]

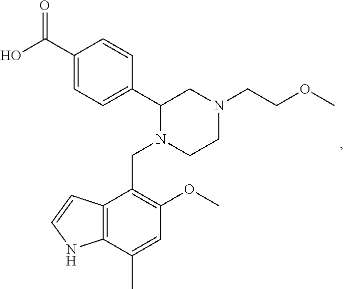 [SMILES: COCCN1CCN(Cc2c(OC)cc(C)c3[nH]ccc23)C(c2ccc(C(=O)O)cc2)C1]

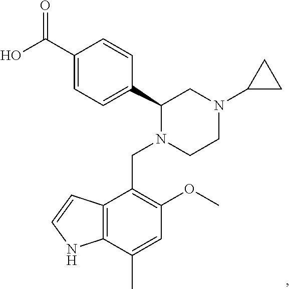 [SMILES: COc1cc(C)c2[nH]ccc2c1CN1CCN(C2CC2)C[C@@H]1c1ccc(C(=O)O)cc1]

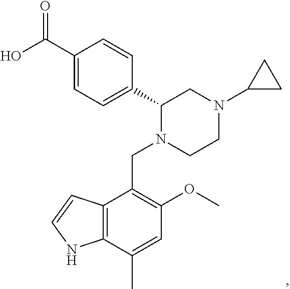 [SMILES: COc1cc(C)c2[nH]ccc2c1CN1CCN(C2CC2)C[C@H]1c1ccc(C(=O)O)cc1]

In some embodiments, compounds of the present disclosure comprise one or more chiral elements, e.g., chiral centers. In some embodiments, compounds are utilized as a mixture of two or more stereoisomers. In some embodiments, compounds are utilized as a mixture of enantiomers. In some embodiments, certain stereoisomer(s) may provide better activities, properties (e.g., lower toxicities), etc. compared to others. In some embodiments, an enantiomer may provide better activities, properties (e.g., lower toxicities), etc. than the other. In some embodiments, single stereoisomers are utilized. In some embodiments, a single enantiomer is utilized.

In some embodiments, a compound has a purity of about 95%-100% (e.g., about 95%, 96%, 97%, 98%, 99%, 99.5%, 99.9% or more, or about 96%-100%, 97%-100%, 98%-100%, 99%-100%, 95-99%, 95%-99.5%, 95%-99.9%, etc). In some embodiments, a compound has a diastereomeric purity of about 95%-100% (e.g., about 95%, 96%, 97%, 98%, 99%, 99.5%, 99.9% or more, or about 96%-100%, 97%-100%, 98%-100%, 99%-100%, 95-99%, 95%-99.5%, 95%-99.9%, etc). In some embodiments, a compound has an enantiomeric purity of about 95%-100% (e.g., about 95%, 96%, 97%, 98%, 99%, 99.5%, 99.9% or more, or about 96%-100%, 97%-100%, 98%-100%, 99%-100%, 95-99%, 95%-99.5%, 95%-99.9%, etc). In some embodiments, a percentage is weight percentage. In some embodiments, a percentage is about or at least about 95%. In some embodiments, a percentage is about or at least about 96%. In some embodiments, a percentage is about or at least about 97%. In some embodiments, a percentage is about or at least about 98%. In some embodiments, a percentage is about or at least about 99%. In some embodiments, a percentage is about or at least about 99.5%. In some embodiments, a percentage is about or at least about 99.9%. In some embodiments, a percentage is about 100%.

### III. Compositions and Administration

In some embodiments, compounds are administered as pharmaceutical compositions. In some embodiments, the present disclosure provides a pharmaceutical composition comprising a provided compound, e.g., a compound of formula I or a pharmaceutically acceptable salt thereof, and a pharmaceutically acceptable carrier. In some embodiments, a composition, e.g. a pharmaceutical composition, delivers a provided compound, e.g., a compound of formula I or a pharmaceutically acceptable salt thereof. In some embodiments, agents, e.g., compounds of formula I or pharmaceutically acceptable salts thereof and compositions are useful for treating various conditions, disorders or diseases, e.g., complement-mediated conditions, disorders or diseases, C3 convertase-mediated conditions, disorders or diseases, etc.

Various technologies, e.g., routes, modes, dosage regimens, etc. may be utilized to administer and/or deliver provided compounds and compositions in accordance with the present disclosure. In some embodiments, a route and/or mode of administration can vary depending upon desired result(s). One with skill in the art, i.e., a physician, is aware that dosage regimens can be adjusted to provide a desired response, e.g., a therapeutic response. Methods of administration include, but are not limited to, intradermal, intramuscular, intraperitoneal, intravenous, subcutaneous, intranasal, epidural, oral, sublingual, intracerebral, intrathecal, intravaginal, transdermal, rectal, by inhalation, or topical, particularly to the ears, nose, eyes, or skin. In some embodiments, a mode of administration is left to the discretion of a practitioner.

In some embodiments, compounds can be incorporated into pharmaceutical compositions. Such pharmaceutical compositions are useful for, among other things, administration and delivery to a subject in vivo or ex vivo. In some embodiments, pharmaceutical compositions also contain a pharmaceutically acceptable carrier or excipient. Such carriers or excipients include any pharmaceutical agent, e.g., a pharmaceutical agent that does not itself induce an immune response harmful to the individual receiving a composition, and which may be administered without undue toxicity. Pharmaceutically acceptable carriers or excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

Compounds in pharmaceutical compositions may be provided as pharmaceutically acceptable salts. In some embodiments, salts can be formed with many acids, including but not limited to, hydrochloric, sulfuric, acetic, lactic, tartaric, malic, succinic, benzenesulfonic, etc. In some embodiments, salts can be formed with bases. In some embodiments, salts are alkali, alkaline earth metal, or ammonium salts, e.g., sodium, calcium, trialkylamine salts, etc.

In some embodiments, salts are more soluble in aqueous or other protonic solvents than corresponding, free acid or base forms. In some embodiments, a pharmaceutical composition may be a lyophilized powder. In some embodiments, a pharmaceutical composition comprises a provided compound, e.g., a compound of formula I or a pharmaceutically acceptable salt thereof dissolved in a pharmaceutically acceptable buffer. In some embodiments, a buffer is a saline buffer. In some embodiments, a buffer has a pH around 7.4.

Pharmaceutical compositions can include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. In some embodiments, pharmaceutical compositions comprise carriers, diluents, or excipients suitable for administration by various routes.

In some embodiments, provided compositions are suitable for parenteral administration. In some embodiments, such compositions comprise aqueous and non-aqueous solutions, suspensions or emulsions of active compounds, which preparations are typically sterile and can be isotonic with blood of intended recipients. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran. Additionally, suspensions of active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, a suspension may also contain suitable stabilizers or agents which increase solubility to allow for the preparation of highly concentrated solutions.

Cosolvents and adjuvants may be added to compositions and formulations. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling can include amount, frequency, and method of administration.

Various pharmaceutical compositions and delivery systems appropriate for compositions, methods and uses of the present disclosure are known in the art (see, e.g., Remington: The Science and Practice of Pharmacy. 21st Edition. Philadelphia, PA. Lippincott Williams & Wilkins, 2005) and can be utilized in accordance with the present disclosure.

In some embodiments, the present disclosure provides methods for introducing provided compounds and compositions into cells, animals or subjects. In some embodiments, such methods include contacting a subject (e.g., a cell or tissue of a subject) with, or administering to a subject (e.g., a subject such as a mammal or human) a provided compound, e.g., a compound of formula I or a salt thereof, or a composition thereof.

A compound or composition described herein can be administered in a sufficient or effective amount to a subject (or a cell, tissue or organ thereof) in need thereof. Doses can vary and may depend upon the type, onset, progression, severity, frequency, duration, or probability of a condition, disorder or disease to which treatment is directed, a clinical endpoint desired, previous or simultaneous treatments, general health, age, gender, race or immunological competency of a subject and other factors that will be appreciated by a skilled artisan. Dose amount, number, frequency or duration may be proportionally increased or reduced, as indicated by efficacy, any adverse side effects, complications or other risk factors of a treatment or therapy and the status of a subject. A skilled artisan will appreciate factors that may influence dosage and timing required to provide an amount sufficient for providing a therapeutic or prophylactic benefit.

A dose to achieve a therapeutic effect will vary based on several factors including, but not limited to: route of administration, amount to achieve a therapeutic effect, the specific condition, disorder or disease treated, any host immune response to administered compound or composition, or stability of administered compound or composition.

An effective amount or a sufficient amount can (but need not) be provided in a single administration, may require multiple administrations, and, can (but need not) be, administered alone or in combination with another composition (e.g., comprising or delivering another therapeutic agent). For example, an amount may be proportionally increased as indicated by the need of a subject, type, status and severity of a condition, disorder or disease treated and/or side effects (if any) of treatment. Amounts considered effective also include amounts that result in a reduction of the use of another treatment, therapeutic regimen or protocol, such as administration of another complement inhibitor described herein.

In some embodiments, pharmaceutical compositions comprise or deliver active ingredients, e.g., compounds of formula I or pharmaceutically acceptable salts thereof, in effective amounts to achieve intended purposes e.g., therapeutic purposes. Various technologies may be utilized to determine therapeutically effective amounts in accordance with the present disclosure. Therapeutic doses can depend on, among other factors, ages and general conditions of subjects, severity of conditions, disorders or diseases (e.g., complement-mediated conditions, disorders or diseases, C3 convertase-mediated conditions, disorders or diseases, etc.), etc. Thus, therapeutically effective amounts in humans may fall in a relatively broad range that may be determined by medical practitioners based on responses of individual patients.

In some embodiments, methods and uses of the present disclosure include delivery and administration systemically, regionally or locally, or by any route, for example, by injection or infusion. In some embodiments, delivery of a pharmaceutical composition in vivo may generally be accomplished via injection using a conventional syringe, although other delivery methods such as convection-enhanced delivery can also be used (see, e.g., U.S. Pat. No. 5,720,720). In some embodiments, compounds and compositions may be delivered subcutaneously, epidermally, intradermally, intrathecally, intraorbitally, intramucosally, intraperitoneally, intravenously, intra-pleurally, intraarterially, orally, intrahepatically, via the portal vein, or intramuscularly. In some embodiments, modes of administration include oral and pulmonary administration, suppositories, and transdermal applications. Clinicians specializing in treating patients with complement-mediated conditions, disorders or diseases may determine optimal routes for administration of compounds and compositions as described herein.

In some embodiments, a compound or composition may be administered to a subject four times a day, three times a day, twice a day, once daily, every 2, 3, 4, 5, or 6 days, weekly, or every 2, 3, or 4 weeks, or even at longer intervals. In some embodiments, a compound or composition is administered to a subject once, e.g., as a single injection or as a single infusion over time (e.g., over 5, 10, 15, 20, 30, 40, 50, 60, 90, 120 minutes, or longer). In some embodiments, a compound or composition is administered to a subject twice, e.g., as two injections (e.g., 2, 4, 6, 8, 10, or 12 hours apart) or as two infusions (e.g., 2, 4, 6, 8, 10, or 12 hours apart). In some embodiments, a subject is monitored before and/or following an administration or treatment for level of C3 expression and/or activity, a complement activity, etc., e.g., as measured using an alternative pathway assay, a classical pathway assay, or both. Suitable assays are known in the art and include, e.g., a hemolysis assay and those described in the Examples.

### IV. Diseases, Disorders, and Conditions

Provided technologies are useful for preventing or treating various conditions, disorders or diseases. In some embodiments, the present disclosure provides methods for preventing a condition, disorder or disease, comprising administering to a subject susceptible thereto an effective amount of a compound, e.g., a compound of formula I or a pharmaceutically acceptable salt. In some embodiments, the present disclosure provides methods for treating a condition, disorder or disease, comprising administering to a subject suffering therefrom a therapeutically effective amount of a compound, e.g., a compound of formula I or a pharmaceutically acceptable salt. In some embodiments, the present disclosure provides a method for reducing C3 convertase activity, comprising contacting a C3 convertase with a provided compound, e.g., a compound of formula I or a pharmaceutically acceptable salt thereof, or a composition thereof. In some embodiments, the present disclosure provides a method for reducing factor B activity, comprising contacting a factor B with a provided compound, e.g., a compound of formula I or a pharmaceutically acceptable salt thereof, or a composition thereof. In some embodiments, the present disclosure provides a method for reducing complement activation in a system, comprising administering to the system a provided compound, e.g., a compound of formula I or a pharmaceutically acceptable salt thereof, or a composition thereof. In some embodiments, the present disclosure provides a method for reducing C3 convertase activity in a system, comprising administering to the system a provided compound, e.g., a compound of formula I or a pharmaceutically acceptable salt thereof, or a composition thereof. In some embodiments, the present disclosure provides a method for reducing factor B activity in a system, comprising administering to the system a provided compound, e.g., a compound of formula I or a pharmaceutically acceptable salt thereof, or a composition thereof. In some embodiments, a system is a plurality of cells, a tissue, organ or organism. In some embodiments, a system is or comprises blood. In some embodiments, a system is an animal. In some embodiments, a system is a human. In some embodiments, a subject is a human.

In some embodiments, a condition, disorder or disease is a complement-mediated condition, disorder or disease. In some embodiments, a condition, disorder or disease is a C3 convertase-mediated condition, disorder or disease. In some embodiments, a condition, disorder or disease is a factor B-mediated condition, disorder or disease. In some embodiments, a condition, disorder or disease is or comprises complement-mediated damage to an organ, tissue, or cells. In some embodiments, a compound or composition is administered in combination with another therapeutic agent, e.g., a complement inhibitor.

### Blood-Related Disorders

In some embodiments, a compound is administered to a subject suffering from, or at risk of, a complement-mediated blood-related disorder, such as paroxysmal nocturnal hemoglobinuria (PNH), atypical hemolytic uremic syndrome (aHUS), autoimmune hemolytic anemia, chronic cold agglutinin disease, HELLP syndrome, and/or warm autoimmune hemolytic anemia. In some embodiments, a compound is administered to a subject suffering from, or at risk of, a complement-mediated disorder that affects the circulatory system. For example, in some embodiments, the disorder is thrombotic microangiopathy (TMA) or a vasculitis (e.g., IgA vasculitis) or other disorder associated with vessel inflammation, e.g., blood vessel and/or lymph vessel inflammation. In some embodiments, a vasculitis is polyarteritis nodosa, hypocomplementemic urticarial vasculitis, pulmonary vasculitis, Wegener's granulomatosis, giant cell arteritis, Churg-Strauss syndrome, microscopic polyangiitis, pauci-immune vasculitis, Henoch-Schonlein purpura, Takayasu's arteritis, Kawasaki disease, or Behcet's disease. In some embodiments, a disorder is TMA secondary to atypical hemolytic uremic syndrome. In some embodiments, a subject is positive for antineutrophil cytoplasmic antibody (ANCA).

### Eye Disorders

In some embodiments, a compound is administered to a subject for treatment of a complement-mediated eye disorder, such as macular degeneration (e.g., age-related macular degeneration (AMD) and Stargardt macular dystrophy), diabetic retinopathy, glaucoma, or uveitis (e.g., posterior uveitis or anterior uveitis). In some embodiments, a subject suffers from or is at risk of AMD. In some embodiments the AMD is neovascular (wet) AMD. In some embodiments the AMD is dry AMD. As will be appreciated by those of ordinary skill in the art, dry AMD encompasses geographic atrophy (GA), intermediate AMD, and early AMD. In some embodiments, a subject with GA is treated in order to slow or halt progression of the disease. For example, in some embodiments, treatment of a subject with GA reduces the rate of retinal cell death. A reduction in the rate of retinal cell death may be evidenced by a reduction in the rate of GA lesion growth in patients treated with a compound, as compared with control (e.g., patients given a sham administration). In some embodiments, a subject has intermediate AMD. In some embodiments, a subject has early AMD. In some embodiments, a subject with intermediate or early AMD is treated in order to slow or halt progression of the disease. For example, in some embodiments, treatment of a subject with intermediate AMD may slow or prevent progression to an advanced form of AMD (neovascular AMD or GA). In some embodiments, treatment of a subject with early AMD may slow or prevent progression to intermediate AMD. In some embodiments an eye has both GA and neovascular AMD. In some embodiments an eye has GA but not wet AMD.

In some embodiments, a subject has an eye disorder characterized by macular degeneration, choroidal neovascularization (CNV), retinal neovascularization (RNV), ocular inflammation, or any combination of the foregoing. Macular degeneration, CNV, RNV, and/or ocular inflammation may be a defining and/or diagnostic feature of the disorder. Exemplary disorders that are characterized by one or more of these features include, but are not limited to, macular degeneration related conditions, diabetic retinopathy, retinopathy of prematurity, proliferative vitreoretinopathy, uveitis, keratitis, conjunctivitis, and scleritis. In some embodiments, a subject is in need of treatment for ocular inflammation. Ocular inflammation can affect a large number of eye structures such as the conjunctiva (conjunctivitis), cornea (keratitis), episclera, sclera (scleritis), uveal tract, retina, vasculature, and/or optic nerve. Evidence of ocular inflammation can include the presence of inflammation-associated cells such as white blood cells (e.g., neutrophils, macrophages) in the eye, the presence of endogenous inflammatory mediator(s), one or more symptoms such as eye pain, redness, light sensitivity, blurred vision and floaters, etc. Uveitis is a general term that refers to inflammation in the uvea of the eye, e.g., in any of the structures of the uvea, including the iris, ciliary body or choroid. Specific types of uveitis include iritis, iridocyclitis, cyclitis, pars planitis and choroiditis. In some embodiments, the eye disorder is Behcet's disease. In some embodiments, the eye disorder is an eye disorder characterized by optic nerve damage (e.g., optic nerve degeneration), such as glaucoma. Additional eye disorders include, e.g., retinitis pigmentosa, macular edema, Vogt-Koyangi-Harada syndrome, birdshot retino-chorioditis, sympathetic ophthalmia, ocular dicatricial pemphigoid, ocular pemphigus, nonartertic ischemic optic neuropathy, post-operative inflammation, and retinal vein occlusion.

### Nervous System Disorders

In some embodiments, a compound is used to treat a subject suffering from or at risk of a complement-mediated disorder that affects the nervous system, e.g., the central nervous system (CNS) and/or peripheral nervous system (PNS). Examples of such disorders include, e.g., a neurodegenerative disorder such as multiple sclerosis, other demyelinating diseases (e.g., neuromyelits optica or chronic inflammatory demyelinating polyneuropathy (CIDP)), amyotrophic lateral sclerosis, chronic pain, fibromyalgia, stroke, intracerebral hemorrhage, allergic neuritis, diabetic neuropathy, Huntington's disease, schizophrenia, Alzheimer's disease, Parkinson's disease, progressive supranuclear palsy, Lewy body dementia (i.e., dementia with Lewy bodies or Parkinson's disease dementia), frontotemporal dementia, progressive supranuclear palsy, corticobasal syndrome, Pick's disease, mild cognitive impairment, traumatic brain injury, traumatic spinal cord injury, multisystem atrophy, chronic traumatic encephalopathy, Creutzfeldt-Jakob disease, Guillain Barre Syndrome, and leptomeningeal metastasis. In some embodiments, a subject suffers from neuropathic pain, e.g., arising from lesions that involve the somatosensory pathways with damage to small fibres in peripheral nerves and/or to the spino-thalamocortical system in the CNS.

### Kidney Disorders

In some embodiments, a compound is used to treat a subject suffering from, or at risk of, a complement-mediated kidney disorder. Such disorders include, e.g., nephritis, e.g., glomerulonephritis, e.g., membranoproliferative glomerulonephritis (MPGN) (e.g., MPGN type I, MPGN type II, or MPGN type III), e.g., immune complex membranoproliferative glomerulonephritis (IC-MPGN). In some embodiments the disorder is IgA nephropathy (IgAN), primary membranous nephropathy, or diabetic nephropathy. In some embodiments, the disorder is polycystic kidney disease (PKD). In some embodiments, the disorder is C3 glomerulopathy. In some embodiments the disorder is characterized by glomerular deposits containing one or more complement activation products, e.g., C3b, in the kidney. In some embodiments treatment as described herein reduces the level of such deposits. In some embodiments a subject suffering from a complement-mediated kidney disorder suffers from proteinuria (an abnormally high level of protein in the urine) and/or an abnormally low glomerular filtration rate (GFR). In some embodiments treatment as described herein results in decreased proteinuria and/or an increased or stabilized GFR.

### Respiratory Disorders

In some embodiments, a compound is used to treat a subject suffering from or at risk of a complement-mediated disorder respiratory disorder. In some embodiments, a subject is suffering from or at risk of acute respiratory distress syndrome. In some embodiments, a respiratory disease is, e.g., asthma (e.g., allergic asthma), emphysema, chronic inflammation, chronic obstructive pulmonary disease (COPD), pulmonary fibrosis (e.g., idiopathic pulmonary fibrosis), radiation-induced lung injury, allergic bronchopulmonary aspergillosis, hypersensitivity pneumonitis (also known as allergic alveolitis), eosinophilic pneumonia, interstitial pneumonia, sarcoid, Wegener's granulomatosis, pulmonary embolisms and infarcts, dyspnea, hemoptysis, bronchoconstriction, or bronchiolitis obliterans.

### Musculoskeletal Disorders

In some embodiments, a compound is used to treat a subject suffering from, or at risk of, a complement-mediated disorder that affects the musculoskeletal system. Examples of such disorders include inflammatory joint conditions (e.g., arthritis such as rheumatoid arthritis or psoriatic arthritis, juvenile chronic arthritis, spondyloarthropathies Reiter's syndrome, gout). In some embodiments, a musculoskeletal system disorder results in symptoms such as pain, stiffness and/or limitation of motion of the affected body part(s). Inflammatory myopathies include dermatomyositis, polymyositis, and various others are disorders of chronic muscle inflammation of unknown etiology that result in muscle weakness. In some embodiments, a complement-mediated musculoskeletal disorder is myasthenia gravis.

### Transplantation

In some embodiments, a compound is used to protect a graft from complement-mediated damage. A graft can be contacted with a compound prior to, during, and/or after being transplanted, in various embodiments of the disclosure. In another embodiment, a compound is administered to a donor prior to removal of the graft. In some embodiments, a compound is administered to a recipient during and/or after the introduction of the graft. In some embodiments, a compound is administered to a recipient prior to the introduction of the graft. In some embodiments, a subject receives a compound after receiving the graft.

In some embodiments, a graft is or comprises a solid organ such as a kidney, liver, lung, pancreas, or heart. In some embodiments, a graft is or comprises bone, cartilage, fascia, tendon, ligament, cornea, sclera, pericardium, skin, heart valve, blood vessel, amniotic membrane, or dura mater. In some embodiments, a graft comprises multiple organs such as a heart-lung or pancreas-kidney graft. In some embodiments, a graft comprises less than a complete organ or tissue. For example, a graft may contain a portion of an organ or tissue, e.g., a liver lobe, section of blood vessel, skin flap, or heart valve. In some embodiments, a graft comprises a preparation comprising isolated cells or tissue fragments that have been isolated from their tissue of origin but retain at least some tissue architecture, e.g., pancreatic islets. In some embodiments, a preparation comprises isolated cells that are not attached to each other via connective tissue, e.g., hematopoietic stem cells or progenitor cells derived from peripheral and/or cord blood, or whole blood or any cell-containing blood product such as red blood cells (RBCs) or platelets.

In some embodiments, a graft is a xenograft (i.e., the donor and recipient are of different species), an autograft (i.e., a graft from one part of the body to another part of the body in the same individual), an isograft (i.e., the donor and recipient are genetically identical), or an allograft (i.e., the donor and recipient are genetically non-identical members of the same species).

### Ischemia/Reperfusion Injury

Ischemia-reperfusion (I/R) injury is an important cause of tissue damage following trauma and in other conditions associated with temporary disruption of blood flow such as myocardial infarction, stroke, severe infection, vascular disease, aneurysm repair, cardiopulmonary bypass, and transplantation. In the setting of trauma, systemic hypoxemia, hypotension, and local interruption of the blood supply resulting from contusions, compartment syndrome, and vascular injuries cause ischemia that damages metabolically active tissues. Restoration of the blood supply triggers an intense systemic inflammatory reaction. After reperfusion, all three major complement pathways are activated and, acting cooperatively or independently, are involved in I/R related adverse events affecting numerous organ systems.

In some embodiments a compound is administered to a subject who has recently (e.g., within the preceding 2, 4, 8, 12, 24, or 48 hours) experienced trauma, e.g., trauma that puts the subject at risk of I/R injury, e.g., due to systemic hypoxemia, hypotension, and/or local interruption of the blood supply. In some embodiments, a compound may be administered intravascularly, optionally into a blood vessel that supplies an injured body part or directly to the body part. In some embodiments, the subject suffers from spinal cord injury, traumatic brain injury, burn, and/or hemorrhagic shock.

In some embodiments, a compound is administered to a subject prior to, during, or after a surgical procedure, e.g., a surgical procedure that is expected to temporarily disrupt blood flow to a tissue, organ, or portion of the body. Examples of such procedures include cardiopulmonary bypass, angioplasty, heart valve repair/replacement, aneurysm repair, or other vascular surgeries. A compound may be administered prior to, after, and/or during an overlapping time period with the surgical procedure.

In some embodiments, a compound is administered to a subject who has suffered an MI, thromboembolic stroke, deep vein thrombosis, or pulmonary embolism. A compound may be administered in combination with a thrombolytic agent such as tissue plasminogen activator (tPA) (e.g., alteplase (Activase), reteplase (Retavase), tenecteplase (TNKase)), anistreplase (Eminase), streptokinase (Kabikinase, Streptase), or urokinase (Abbokinase). In some embodiments, a compound may be administered prior to, after, and/or during an overlapping time period with the thrombolytic agent.

### Other Disorders

In some embodiments, a compound is used to treat a subject suffering from, or at risk of, a complement-mediated disorder that affects the integumentary system. Examples of such disorders include, e.g., atopic dermatitis, psoriasis, pemphigoid, pemphigus, systemic lupus erythematosus, dermatomyositis, scleroderma, sclerodermatomyositis, Sjögren syndrome, and chronic urticaria.

In some embodiments, a compound is used to treat a subject suffering from, or at risk of, a complement-mediated disorder that affects the gastrointestinal system, e.g., inflammatory bowel disease, e.g., Crohn's disease or ulcerative colitis.

In some embodiments, a compound is used to treat a subject suffering from, or at risk of, a complement-mediated inflammatory disorder, such as rhinosinusitis or myocarditis.

In some embodiments, a compound is used to treat a subject suffering from, or at risk of, thyroiditis (e.g., Hashimoto's thyroiditis, Graves' disease, post-partum thyroiditis), hepatitis (e.g., hepatitis C), pancreatitis, panniculitis, or MYH9-related disorders.

In some embodiments, a compound is used to treat interleukin-2 induced toxicity during IL-2 therapy, myocardial infarction, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, liver fibrosis, fibrogenic dust diseases, nasal polyposis, parasitic diseases, Goodpasture's Syndrome, immune complex-associated inflammation, antiphospholipid syndrome, cancer, periodontitis, gingivitis, or obesity.

In some embodiments, a complement-mediated condition, disorder or disease is complement activation secondary to administration of another therapeutic or diagnostic agent. For example, in some embodiments, a complement-mediated condition, disorder or disease is complement activation secondary to gene therapy (e.g., gene therapy with a viral vector such as an adeno-associated virus (AAV), adenovirus, or lentivirus vector) or complement activation secondary to cell therapy). In some embodiments, a subject suffers from TMA secondary to hematopoietic stem cell transplant (HSCT-TMA). In some embodiments, a subject suffers from drug-induced TMA. In some embodiments, administration of a compound described herein prior to and/or following administration of another therapeutic agent may increase efficacy and/or safety of said therapeutic agent.

### V. Combination Therapy

In some embodiments, provided technologies comprise administering a compound, e.g., a compound of formula I or a pharmaceutically acceptable salt thereof, in combination with another therapy or therapeutic agent. In some embodiments, provided technologies comprise administering a compound, e.g., a compound of formula I or a pharmaceutically acceptable salt thereof, in combination with one or more additional complement inhibitors. In some embodiments, a provided compound or composition is administered to a subject already receiving therapy with another complement inhibitor. In some embodiments, another complement inhibitor is administered to a subject receiving a provided compound, e.g., a compound of formula I or a pharmaceutically acceptable salt thereof, or a composition thereof. In some embodiments, both a provided compound, e.g., a compound of formula I or a pharmaceutically acceptable salt thereof and another complement inhibitor are administered to a subject.

In some embodiments, administration of a provided compound, e.g., a compound of formula I or a pharmaceutically acceptable salt thereof may allow for administering a reduced dosing regimen of (e.g., involving a smaller amount in an individual dose, reduced frequency of dosing, reduced number of doses, and/or reduced overall exposure to) a second therapeutic agent, e.g., a complement inhibitor, as compared to administration of a second therapeutic agent, e.g., a complement inhibitor, as single therapy. Without wishing to be bound by any theory, in some embodiments a reduced dosing regimen of a second therapeutic agent, e.g., a complement inhibitor, may avoid one or more undesired adverse effects that could otherwise result.

In some aspects, administration of a provided compound, e.g., a compound of formula I or a pharmaceutically acceptable salt thereof in combination with a second therapeutic agent, e.g., a complement inhibitor can reduce the level of C3 activity in a subject's blood sufficiently such that a reduced dosing regimen of a provided compound and/or the second therapeutic agent is required to achieve a desired degree of complement inhibition.

In some embodiments such a reduced dose can be administered in a smaller volume, or using a lower concentration, or using a longer dosing interval, or any combination of the foregoing, as compared to administration of a provided compound, e.g., a compound of formula I or a pharmaceutically acceptable salt thereof or a second therapeutic agent, e.g., a complement inhibitor as single therapy.

### A. Additional Complement Inhibitors

In some aspects, methods of the present disclosure involve administering a compound described herein, alone or in combination with one or more additional complement inhibitors. In some embodiments, a compound is administered to a subject already receiving therapy with another complement inhibitor; in some embodiments, another complement inhibitor is administered to a subject receiving a compound. In some embodiments, both a compound and another complement inhibitor are administered to the subject.

In some embodiments administration of a compound may allow for administering a reduced dosing regimen of (e.g., involving a smaller amount in an individual dose, reduced frequency of dosing, reduced number of doses, and/or reduced overall exposure to) a second complement inhibitor, as compared to administration of a second complement inhibitor as single therapy. Without wishing to be bound by any theory, in some embodiments a reduced dosing regimen of a second complement inhibitor may avoid one or more undesired adverse effects that could otherwise result.

In some aspects, administration of a compound in combination with a second complement inhibitor can reduce the amount of C3 in the subject's blood sufficiently such that a reduced dosing regimen of a compound and/or the second complement inhibitor is required to achieve a desired degree of complement inhibition.

In some aspects, administration of a compound in combination with a second complement inhibitor can reduce the amount of C3 in the subject's blood sufficiently such that a reduced dosing regimen of a compound and/or the second complement inhibitor is required to achieve a desired level of, or a desired amount of improvement in, one or more signs, symptoms, biomarkers, or outcome measures, of a complement-mediated disorder.

In some embodiments such a reduced dose can be administered in a smaller volume, or using a lower concentration, or using a longer dosing interval, or any combination of the foregoing, as compared to administration of a compound or a second complement inhibitor as single therapy.

Any complement inhibitor, e.g., a complement inhibitor known in the art, can be administered in combination with a compound described herein. In some embodiments, a complement inhibitor is compstatin or a compstatin analog.

Compstatin is a cyclic peptide that binds to C3 and inhibits complement activation. U.S. Pat. No. 6,319,897 describes a peptide having the sequence Ile-[Cys-Val-Val-Gln-Asp-Trp-Gly-His-His-Arg-Cys]-Thr (SEQ ID NO: 1), with the disulfide bond between the two cysteines denoted by brackets. It will be understood that the name “compstatin” was not used in U.S. Pat. No. 6,319,897 but was subsequently adopted in the scientific and patent literature (see, e.g., Morikis, et al., Protein Sci., 7(3):619-27, 1998) to refer to a peptide having the same sequence as SEQ ID NO: 2 disclosed in U.S. Pat. No. 6,319,897, but amidated at the C terminus. The term “compstatin” is used herein consistently with such usage. Compstatin analogs that have higher complement inhibiting activity than compstatin have been developed. See, e.g., WO2004/026328 (PCT/US2003/029653), Morikis, D., et al., Biochem Soc Trans. 32(Pt 1):28-32, 2004, Mallik, B., et al., J. Med. Chem., 274-286, 2005; Katragadda, M., et al. J. Med. Chem., 49: 4616-4622, 2006; WO2007062249 (PCT/US2006/045539); WO2007044668 (PCT/US2006/039397), WO/2009/046198 (PCT/US2008/078593); WO/2010/127336 (PCT/US2010/033345).

As used herein, the term “compstatin analog” includes compstatin and any complement inhibiting analog thereof. The term “compstatin analog” encompasses compstatin and other compounds designed or identified based on compstatin and whose complement inhibiting activity is at least 50% as great as that of compstatin as measured, e.g., using any complement activation assay accepted in the art or substantially similar or equivalent assays. Certain suitable assays are described in U.S. Pat. No. 6,319,897, WO2004/026328, Morikis, supra, Mallik, supra, Katragadda 2006, supra, WO2007062249 (PCT/US2006/045539); WO2007044668 (PCT/US2006/039397), WO/2009/046198 (PCT/US2008/078593); and/or WO/2010/127336 (PCT/US2010/033345). The assay may, for example, measure alternative or classical pathway-mediated erythrocyte lysis or be an ELISA assay. In some embodiments, an assay described in WO/2010/135717 (PCT/US2010/035871) is used.

Table 1 provides a non-limiting list of compstatin analogs useful in the present disclosure. The analogs are referred to in abbreviated form in the left column by indicating specific modifications at designated positions (1-13) as compared to the parent peptide, compstatin. Consistent with usage in the art, “compstatin” as used herein, and the activities of compstatin analogs described herein relative to that of compstatin, refer to the compstatin peptide amidated at the C-terminus. Unless otherwise indicated, peptides in Table 1 are amidated at the C-terminus. Bold text is used to indicate certain modifications. Activity relative to compstatin is based on published data and assays described therein (WO2004/026328, WO2007044668, Mallik, 2005; Katragadda, 2006). In certain embodiments, the peptides listed in Table 1 are cyclized via a disulfide bond between the two Cys residues when used in the therapeutic compositions and methods of the disclosure. Alternate means for cyclizing the peptides are also within the scope of the disclosure.

TABLE 1

| | | SEQ ID | Activity over |
|---|---|---|---|
| Peptide | Sequence | NO: | compstatin |
| Compstatin | H-ICVVQDWGHHRCT-CONH2 | 8 | * |
| Ac-compstatin | Ac-ICWVQDWGHHRCT-CONH2 | 9 | 3xmore |
| Ac-V4Y/H9A | Ac-ICVYQDWGAHRCT-CONH2 | 10 | 14xmore |
| Ac-V4W/H9A-OH | Ac-ICVWQDWGAHRCT-COOH | 11 | 27xmore |
| Ac-V4W/H9A | Ac-ICVWQDWGAHRCT-CONH2 | 12 | 45xmore |
| Ac-V4W/H9A/T13dT-OH | Ac-ICVWQDWGAHRCdT-COOH | 13 | 55xmore |
| Ac-V4(2-Nal)/H9A | Ac-ICV(2-Nal)QDWGAHRCT-CONH2 | 14 | 99xmore |
| Ac V4(2-Nal)/H9A-OH | Ac-ICV(2-Nal)QDWGAHRCT-COOH | 15 | 38xmore |
| Ac V4(1-Nal)/H9A-OH | Ac-ICV(1-Nal)QDWGAHRCT-COOH | 16 | 30xmore |
| Ac-V42Igl/H9A | Ac-ICV(2-Igl)QDWGAHRCT-CONH2 | 17 | 39xmore |
| Ac-V42Igl/H9A-OH | Ac-ICV(2-Igl)QDWGAHRCT-COOH | 18 | 37xmore |
| Ac-V4Dht/H9A-OH | Ac-ICVDhtQDWGAHRCT-COOH | 19 | 5xmore |
| Ac-V4(Bpa)/H9A-OH | Ac-ICV(Bpa)QDWGAHRCT-COOH | 20 | 49xmore |
| Ac-V4(Bpa)/H9A | Ac-ICV(Bpa)QDWGAHRCT-CONH2 | 21 | 86xmore |
| Ac-V4(Bta)/H9A-OH | Ac-ICV(Bta)QDWGAHRCT-COOH | 22 | 65xmore |
| Ac-V4(Bta)/H9A | Ac-ICV(Bta)QDWGAHRCT-CONH₂ | 23 | 64xmore |
| Ac-V4W/H9(2-Abu) | Ac-ICVWQDWG(2-Abu)HRCT-CONH₂ | 24 | 64xmore |
| +G/V4W/H9A + AN-OH | H-GICVWQDWGAHRCTAN-COOH | 25 | 38xmore |
| Ac-V4(5fW)/H9A | Ac-ICV(5fW)QDWGAHRCT-CONH₂ | 26 | 31xmore |
| Ac-V4(5-MeW)/H9A | Ac-ICV(5-methyl-W)QDWGAHRCT-CONH₂ | 27 | 67xmore |
| Ac-V4(1-MeW)/H9A | Ac-ICV(1-methyl-W)QDWGAHRCT-CONH₂ | 28 | 264xmore |
| Ac-V4W/W7(5fW)/H9A | Ac-ICVWQD(5fW)GAHRCT-CONH₂ | 29 | 121xmore |
| Ac-V4(5fW)/W7(5fW)/H9A | Ac-ICV(5fW)QD(5fW)GAHRCT-CONH₂ | 30 | NA |
| Ac-V4(5-MeW)/W7(5fW)H9A | Ac-ICV(5-methyl-W)QD(5fW)GAHRCT- | 31 | NA |
| | CONH₂ | | |
| Ac-V4(1MeW)/W7(5fW)/H9A | Ac-ICV(1-methyl-W)QD(5fW)GAHRCT- | 32 | 264xmore |
| | CONH₂ | | |
| +G/V4(6fW)/W7(6fW)H9A + N- | H-GICV(6fW)QD(6fW)GAHRCTN-COOH | 33 | 126xmore |
| OH | | | |
| Ac-V4(1-formyl-W)/H9A | Ac-ICV(1-formyl-W)QDWGAHRCT-CONH₂ | 34 | 264xmore |
| Ac-V4(5-methoxy-W)/H9A | Ac-ICV(1-methyoxy-W)QDWGAHRCT- | 35 | 76xmore |
| | CONH₂ | | |
| G/V4(5f-W)/WV7(5fW)/H9A + | H-GICV(5fW)QD(5fW)GAHRCTN-COOH | 36 | 112xmore |
| N-OH | | | |

NA = not available

In certain embodiments of the compositions and methods of the disclosure, the compstatin analog has a sequence selected from sequences 9-36. In one embodiment, the compstatin analog has a sequence of SEQ ID NO: 28. As used herein, “L-amino acid” refers to any of the naturally occurring levorotatory alpha-amino acids normally present in proteins or the alkyl esters of those alpha-amino acids. The term “D-amino acid” refers to dextrorotatory alpha-amino acids. Unless specified otherwise, all amino acids referred to herein are L-amino acids.

In some embodiments, one or more amino acid(s) of a compstatin analog (e.g., any of the compstatin analogs disclosed herein) can be an N-alkyl amino acid (e.g., an N-methyl amino acid). For example, and without limitation, at least one amino acid within the cyclic portion of the peptide, at least one amino acid N-terminal to the cyclic portion, and/or at least one amino acid C-terminal to the cyclic portion may be an N-alkyl amino acid, e.g., an N-methyl amino acid. In some embodiments, for example, a compstatin analog comprises an N-methyl glycine, e.g., at the position corresponding to position 8 of compstatin and/or at the position corresponding to position 13 of compstatin. In some embodiments, one or more of the compstatin analogs in Table 1 contains at least one N-methyl glycine, e.g., at the position corresponding to position 8 of compstatin and/or at the position corresponding to position 13 of compstatin. In some embodiments, one or more of the compstatin analogs in Table 1 contains at least one N-methyl isoleucine, e.g., at the position corresponding to position 13 of compstatin. For example, a Thr at or near the C-terminal end of a peptide whose sequence is listed in Table 1 or any other compstatin analog sequence may be replaced by N-methyl Ile. As will be appreciated, in some embodiments the N-methylated amino acids comprise N-methyl Gly at position 8 and N-methyl Ile at position 13. In some embodiments, a compstatin analog (e.g., any one of the compstatin analogs listed in Table 1) comprises an isoleucine at position corresponding to position 3 of SEQ ID NO: 8, either instead of or in addition to one or more substitutions described herein. For example, in some embodiments, a compstatin analog comprises or consists of the sequence of any one of SEQ ID NOs: 8-36, where position 3 is an isoleucine. In some embodiments, a compstatin analog comprises or consists of the sequence of any one of SEQ ID NOs: 25, 33, or 36, where position 4 is an isoleucine. Additional compstatin analogs are described in, e.g., WO2019/166411.

Compstatin analogs may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies as described in Malik, supra, Katragadda, supra, WO2004026328, and/or WO2007062249. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3^{rd} ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g. to physiological pH, using a base such as NaOH. Peptide preparations may be characterized by mass spectrometry if desired, e.g., to confirm mass and/or disulfide bond formation. See, e.g., Mallik, 2005, and Katragadda, 2006.

A compstatin analog can be modified by addition of a molecule such as polyethylene glycol (PEG) to stabilize the compound, reduce its immunogenicity, increase its lifetime in the body, increase or decrease its solubility, and/or increase its resistance to degradation. Methods for pegylation are well known in the art (Veronese, F. M. & Harris, Adv. Drug Deliv. Rev. 54, 453-456, 2002; Davis, F. F., Adv. Drug Deliv. Rev. 54, 457-458, 2002); Hinds, K. D. & Kim, S. W. Adv. Drug Deliv. Rev. 54, 505-530 (2002; Roberts, M. J., Bentley, M. D. & Harris, J. M. Adv. Drug Deliv. Rev. 54, 459-476; 2002); Wang, Y. S. et al. Adv. Drug Deliv. Rev. 54, 547-570, 2002). A wide variety of polymers such as PEGs and modified PEGs, including derivatized PEGs to which polypeptides can conveniently be attached are described in Nektar Advanced Pegylation 2005-2006 Product Catalog, Nektar Therapeutics, San Carlos, CA, which also provides details of appropriate conjugation procedures.

In some embodiments, a compstatin analog of any of SEQ ID NOs: 9-36, is extended by one or more amino acids at the N-terminus, C-terminus, or both, wherein at least one of the amino acids has a side chain that comprises a reactive functional group such as a primary or secondary amine, a sulfhydryl group, a carboxyl group (which may be present as a carboxylate group), a guanidino group, a phenol group, an indole ring, a thioether, or an imidazole ring, which facilitate conjugation with a reactive functional group to attach a PEG to the compstatin analog. In some embodiments, the compstatin analog comprises an amino acid having a side chain comprising a primary or secondary amine, e.g., a Lys residue. For example, a Lys residue, or a sequence comprising a Lys residue, is added at the N-terminus and/or C-terminus of a compstatin analog described herein (e.g., a compstatin analog comprising any one of SEQ ID NOs: 9-36).

In some embodiments, the Lys residue is separated from the cyclic portion of the compstatin analog by a rigid or flexible spacer. The spacer may, for example, comprise a substituted or unsubstituted, saturated or unsaturated alkyl chain, oligo(ethylene glycol) chain, and/or other moieties, e.g., as described herein with regard to linkers. The length of the chain may be, e.g., between 2 and 20 carbon atoms. In other embodiments the spacer is a peptide. The peptide spacer may be, e.g., between 1 and 20 amino acids in length, e.g., between 4 and 20 amino acids in length. Suitable spacers can comprise or consist of multiple Gly residues, Ser residues, or both, for example. Optionally, the amino acid having a side chain comprising a primary or secondary amine and/or at least one amino acid in a spacer is a D-amino acid. Any of a variety of polymeric backbones or scaffolds could be used. For example, the polymeric backbone or scaffold may be a polyamide, polysaccharide, polyanhydride, polyacrylamide, polymethacrylate, polypeptide, polyethylene oxide, or dendrimer. Suitable methods and polymeric backbones are described, e.g., in WO98/46270 (PCT/US98/07171) or WO98/47002 (PCT/US98/06963). In one embodiment, the polymeric backbone or scaffold comprises multiple reactive functional groups, such as carboxylic acids, anhydride, or succinimide groups. The polymeric backbone or scaffold is reacted with the compstatin analogs. In one embodiment, the compstatin analog comprises any of a number of different reactive functional groups, such as carboxylic acids, anhydride, or succinimide groups, which are reacted with appropriate groups on the polymeric backbone. Alternately, monomeric units that could be joined to one another to form a polymeric backbone or scaffold are first reacted with the compstatin analogs and the resulting monomers are polymerized. In another embodiment, short chains are prepolymerized, functionalized, and then a mixture of short chains of different composition are assembled into longer polymers.

In some embodiments, a compstatin analog moiety is attached at each end of a linear PEG. A bifunctional PEG having a reactive functional group at each end of the chain may be used, e.g., as described herein. In some embodiments, the reactive functional groups are identical while in some embodiments different reactive functional groups are present at each end.

In general and compounds depicted herein, a polyethylene glycol moiety is drawn with the oxygen atom on the right side of the repeating unit or the left side of the repeating unit. In cases where only one orientation is drawn, the present disclosure encompasses both orientations (i.e., (CH₂CH₂O)ₙ and (OCH₂CH₂)ₙ) of polyethylene glycol moieties for a given compound or genus, or in cases where a compound or genus contains multiple polyethylene glycol moieties, all combinations of orientations are encompasses by the present disclosure.

In some embodiments a bifunctional linear PEG comprises a moiety comprising a reactive functional group at each of its ends. The reactive functional groups may be the same (homobifunctional) or different (heterobifunctional). In some embodiments the structure of a bifunctional PEG may be symmetric, wherein the same moiety is used to connect the reactive functional group to oxygen atoms at each end of the —(CH₂CH₂O)ₙ chain. In some embodiments different moieties are used to connect the two reactive functional groups to the PEG portion of the molecule. The structures of exemplary bifunctional PEGs are depicted below. For illustrative purposes, formulas in which the reactive functional group(s) comprise an NHS ester are depicted, but other reactive functional groups could be used.

In some embodiments, a bifunctional linear PEG is of formula A:

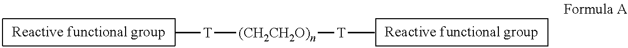

wherein each T and “Reactive functional group” is independently as defined below, and described in classes and subclasses herein, and n is as defined above and described in classes and subclasses herein.
Each T is independently a covalent bond or a C₁₋₁₂ straight or branched, hydrocarbon chain wherein one or more carbon units of T are optionally and independently replaced by —O—, —S—, —N(R^{x})—, —C(O)—, —C(O)O—, —OC(O)—, —N(R^{x})C(O)—, —C(O)N(R^{x})—, —S(O)—, —S(O)₂—, —N(R^{x})SO₂—, or —SO₂N(R^{x})—; and
each R^{x} is independently hydrogen or C₁₋₆ aliphatic.
   The Reactive functional group has the structure —COO—NHS.

Exemplary bifunctional PEGs of formula A include:

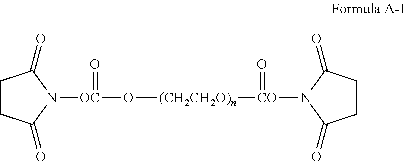

In some embodiments, a functional group (for example, an amine, hydroxyl, or thiol group) on a compstatin analog is reacted with a PEG-containing compound having a “reactive functional group” as described herein, to generate such conjugates. By way of example, Formula A-I can form compstatin analog conjugates having the structure:

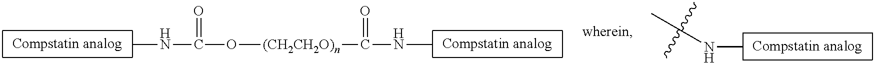

represents the attachment point of an amine group on a compstatin analog. In certain embodiments, an amine group is a lysine side chain group.

In certain embodiments, the PEG component of such conjugates has an average molecular weight of about 5 kD, about 10 kD, about 15 kD, about 20 kD, about 30 kD, or about 40 kD. In certain embodiments, the PEG component of such conjugates has an average molecular weight of about 40 kD.

The term “bifunctional” or “bifunctionalized” is sometimes used herein to refer to a compound comprising two compstatin analog moieties linked to a PEG. Such compounds may be designated with the letter “BF”. In some embodiments a bifunctionalized compound is symmetrical. In some embodiments the linkages between the PEG and each of the compstatin analog moieties of a bifunctionalized compound are the same. In some embodiments, each linkage between a PEG and a compstatin analog of a bifunctionalized compound comprises a carbamate. In some embodiments, each linkage between a PEG and a compstatin analog of a bifunctionalized compound comprises a carbamate and does not comprise an ester. In some embodiments, each compstatin analog of a bifunctionalized compound is directly linked to a PEG via a carbamate. In some embodiments, each compstatin analog of a bifunctionalized compound is directly linked to a PEG via a carbamate, and the bifunctionalized compound has the structure:

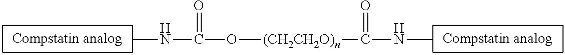

In some embodiments of formulae and embodiments described herein,

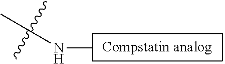 [SMILES: *NC]

represents point of attachment of a lysine side chain group in a compstatin analog having the structure:

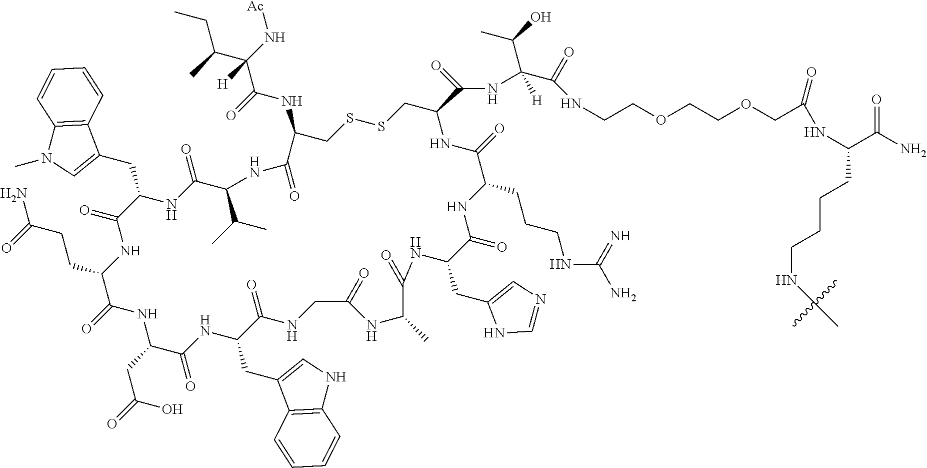 [SMILES: *NCCCC[C@H](NC(=O)COCCOCCNC(=O)[C@@H](NC(=O)[C@@H]1CSSC[C@H](NC(=O)[C@@H](NC(C)=O)[C@@H](C)CC)C(=O)N[C@@H](C(C)C)C(=O)N[C@@H](Cc2cn(C)c3ccccc23)C(=O)N[C@@H](CCC(N)=O)C(=O)N[C@@H](CC(=O)O)C(=O)N[C@@H](Cc2c[nH]c3ccccc23)C(=O)NCC(=O)N[C@@H](C)C(=O)N[C@@H](Cc2cnc[nH]2)C(=O)N[C@@H](CCCNC(=N)N)C(=O)N1)[C@@H](C)O)C(N)=O]

wherein the symbol “ [symbol] ” denotes the point of attachment of a chemical moiety to the remainder of a molecule or chemical formula.

PEGs comprising one or more reactive functional groups may, in some embodiments, be obtained from, e.g., NOF America Corp. White Plains, NY or BOC Sciences 45-16 Ramsey Road Shirley, NY 11967, USA, among others, or may be prepared using methods known in the art.

In some embodiments, a linker is used to connect a compstatin analog described herein and a PEG described herein. Suitable linkers for connecting a compstatin analog and a PEG are extensively described above and in classes and subclasses herein. In some embodiments, a linker has multiple functional groups, wherein one functional group is connected to a compstatin analog and another is connected to a PEG moiety. In some embodiments, a linker is a bifunctional compound. In some embodiments, a linker has the structure of NH₂(CH₂CH₂O)ₙCH₂C(═O)OH, wherein n is 1 to 1000. In some embodiments, a linker is 8-amino-3,6-dioxaoctanoic acid (AEEAc). In some embodiments, a linker is activated for conjugation with a polymer moiety or a functional group of a compstatin analog. For example, in some embodiments, the carboxyl group of AEEAc is activated before conjugation with the amine group of the side chain of a lysine group.

In some embodiments, a suitable functional group (for example, an amine, hydroxyl, thiol, or carboxylic acid group) on a compstatin analog is used for conjugation with a PEG moiety, either directly or via a linker. In some embodiments, a compstatin analog is conjugated through an amine group to a PEG moiety via a linker. In some embodiments, an amine group is the a-amino group of an amino acid residue. In some embodiments, an amine group is the amine group of the lysine side chain. In some embodiments, a compstatin analog is conjugated to a PEG moiety through the amino group of a lysine side chain (s-amino group) via a linker having the structure of NH₂(CH₂CH₂O)ₙCH₂C(═O)OH, wherein n is 1 to 1000. In some embodiments, a compstatin analog is conjugated to the PEG moiety through the amino group of a lysine side chain via an AEEAc linker. In some embodiments, the NH₂(CH₂CH₂O)ₙCH₂C(═O)OH linker introduces a —NH(CH₂CH₂O)ₙCH₂C(═O)— moiety on a compstatin lysine side chain after conjugation. In some embodiments, the AEEAc linker introduces a —NH(CH₂CH₂O)₂CH₂C(═O)— moiety on a compstatin lysine side chain after conjugation.

In some embodiments, a compstatin analog is conjugated to a PEG moiety via a linker, wherein the linker comprises an AEEAc moiety and an amino acid residue. In some embodiments, a compstatin analog is conjugated to a PEG moiety via a linker, wherein the linker comprises an AEEAc moiety and a lysine residue. In some embodiments, the C-terminus of a compstatin analog is connected to the amino group of AEEAc, and the C-terminus of AEEAc is connected to a lysine residue. In some embodiments, the C-terminus of a compstatin analog is connected to the amino group of AEEAc, and the C-terminus of AEEAc is connected to the a-amino group of a lysine residue. In some embodiments, the C-terminus of a compstatin analog is connected to the amino group of AEEAc, the C-terminus of AEEAc is connected to the a-amino group of the lysine residue, and a PEG moiety is conjugated through the F-amino group of said lysine residue. In some embodiments, the C-terminus of the lysine residue is modified. In some embodiments, the C-terminus of the lysine residue is modified by amidation. In some embodiments, the N-terminus of a compstatin analog is modified. In some embodiments, the N-terminus of a compstatin analog is acetylated.

In certain embodiments a compstatin analog may be represented as M-AEEAc-Lys-B₂, wherein B₂ is a blocking moiety, e.g., NH₂, M represents any of SEQ ID NOs: 9-36, with the proviso that the C-terminal amino acid of any of SEQ ID NOs: 9-36 is linked via a peptide bond to AEEAc-Lys-B₂. The NHS moiety of a monofunctional or multifunctional (e.g., bifunctional) PEG reacts with the free amine of the lysine side chain to generate a monofunctionalized (one compstatin analog moiety) or multifunctionalized (multiple compstatin analog moieties) PEGylated compstatin analog. In various embodiments any amino acid comprising a side chain that comprises a reactive functional group may be used instead of Lys (or in addition to Lys). A monofunctional or multifunctional PEG comprising a suitable reactive functional group may be reacted with such side chain in a manner analogous to the reaction of NHS-ester activated PEGs with Lys.

With regard to any of the above formulae and structures, it is to be understood that embodiments in which the compstatin analog component comprises any compstatin analog described herein, e.g., any compstatin analog of SEQ ID NOs; 9-36 are expressly disclosed. For example, and without limitation, a compstatin analog may comprise the amino acid sequence of SEQ ID NO: 28. An exemplary PEGylated compstatin analog in which the compstatin analog component comprises the amino acid sequence of SEQ ID NO: 28 is depicted below:

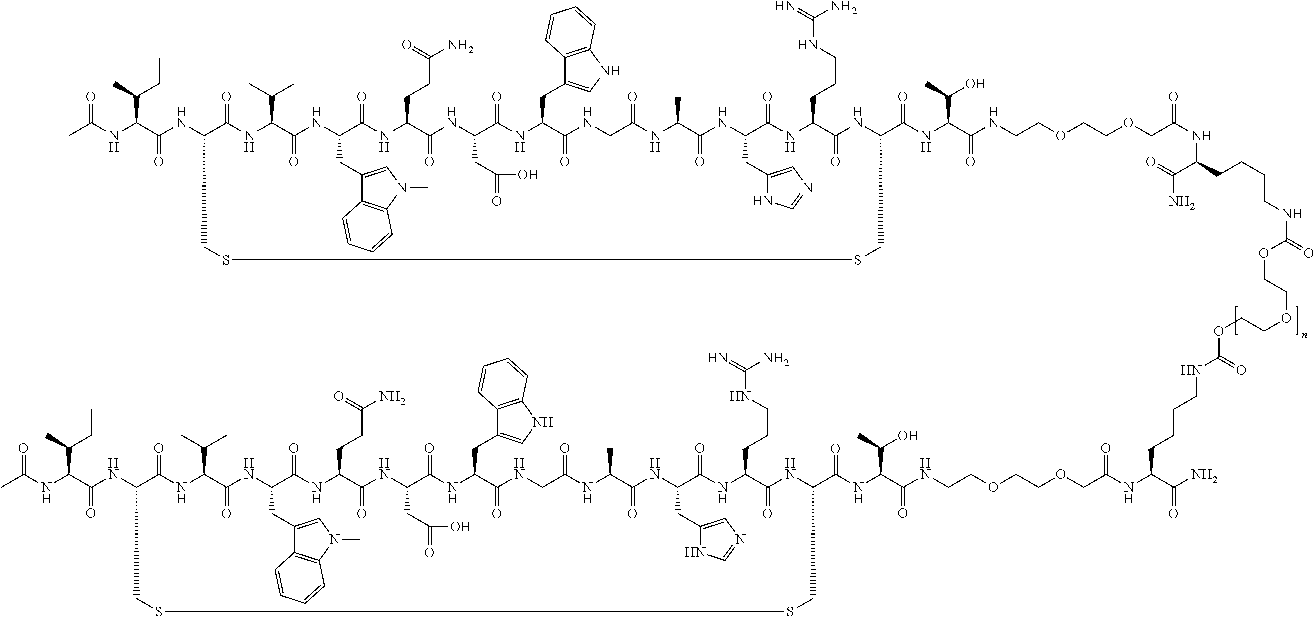

It will be understood that the PEG moiety may have a variety of different molecular weights or average molecular weights in various embodiments, as described herein. In certain embodiments, a compstatin analog is pegcetacoplan (“APL-2”), having the structure above with n of about 800 to about 1100 and a PEG having an average molecular weight of about 40 kD. Pegcetacoplan is also referred to as Poly(oxy-1,2-ethanediyl), α-hydro-ω-hydroxy-, 15,15′-diester with N-acetyl-L-isoleucyl-L-cysteinyl-L-valyl-1-methyl-L-tryptophyl-L-glutaminyl-L-a-aspartyl-L-tryptophylglycyl-L-alanyl-L-histidyl-L-arginyl-L-cysteinyl-L-threonyl-2-[2-(2-aminoethoxy)ethoxy]acetyl-N⁶-carboxy-L-lysinamide cyclic (2→12)-(disulfide); or 0,0′-bis[(S²,S¹²-cyclo{N-acetyl-L-isoleucyl-L-cysteinyl-L-valyl-1-methyl-L-tryptophyl-L-glutaminyl-L-a-aspartyl-L-tryptophylglycyl-L-alanyl-L-histidyl-L-arginyl-L-cysteinyl-L-threonyl-2-[2-(2-aminoethoxy)ethoxy]acetyl-L-lysinamide})-N^{6,15}-carbonyl]polyethylene glycol (n=800-1100). Additional compstatin analogs are described in, e.g., WO 2012/155107 and WO 2014/078731.

In some embodiments, a compstatin analog described herein is administered twice weekly or every 3 days, at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg, for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer.

In some embodiments, a composition comprising a provided compound is administered to a subject in combination with a compstatin analog, such that the compstatin analog and/or the composition is administered less frequently and/or at a lower dosage. In some embodiments, a composition comprising a provided compound is administered to a subject in combination with a compstatin analog, such that the compstatin analog is administered once a week, once every 2 weeks, once a month, once every 2 months, 3 months, 4 months, 5 months, or longer, at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg.

In some embodiments, a complement inhibitor is an antibody, e.g., an anti-C3 and/or anti-C5 antibody, or a fragment thereof. In some embodiments, an antibody fragment may be used to inhibit C3 or C5 activation. The fragmented anti-C3 or anti-C5 antibody may be Fab′, Fab′(2), Fv, or single chain Fv. In some embodiments, the anti-C3 or anti-C5 antibody is monoclonal. In some embodiments, the anti-C3 or anti-C5 antibody is polyclonal. In some embodiments, the anti-C3 or anti-C5 antibody is de-immunized. In some embodiments the anti-C3 or anti-C5 antibody is a fully human monoclonal antibody. In some embodiments, the anti-C5 antibody is eculizumab. In some embodiments, a complement inhibitor is an antibody, e.g., an anti-C3 and/or anti-C5 antibody, or a fragment thereof.

In some embodiments, a complement inhibitor is a polypeptide inhibitor and/or a nucleic acid aptamer (see, e.g., U.S. Publ. No. 20030191084). Exemplary polypeptide inhibitors include an enzyme that degrades C3 or C3b (see, e.g., U.S. Pat. No. 6,676,943). Additional polypeptide inhibitors include mini-factor H (see, e.g., U.S. Publ. No. 20150110766), Efb protein or complement inhibitor (SCIN) protein from Staphylococcus aureus, or a variant or derivative or mimetic thereof (see, e.g., U.S. Publ. 20140371133).

A variety of other complement inhibitors can also be used in various embodiments of the disclosure. In some embodiments, the complement inhibitor is a naturally occurring mammalian complement regulatory protein or a fragment or derivative thereof. For example, the complement regulatory protein may be CR1, DAF, MCP, CFH, or CFI. In some embodiments, the complement regulatory polypeptide is one that is normally membrane-bound in its naturally occurring state. In some embodiments, a fragment of such polypeptide that lacks some or all of a transmembrane and/or intracellular domain is used. Soluble forms of complement receptor 1 (sCR1), for example, can also be used. For example the compounds known as TP10 or TP20 (Avant Therapeutics) can be used. C1 inhibitor (C1-INH) can also be used. In some embodiments a soluble complement control protein, e.g., CFH, is used.

Inhibitors of C1s can also be used. For example, U.S. Pat. No. 6,515,002 describes compounds (furanyl and thienyl amidines, heterocyclic amidines, and guanidines) that inhibit C1s. U.S. Pat. Nos. 6,515,002 and 7,138,530 describe heterocyclic amidines that inhibit C1s. U.S. Pat. No. 7,049,282 describes peptides that inhibit classical pathway activation. Certain of the peptides comprise or consist of WESNGQPENN (SEQ ID NO: 73) or KTISKAKGQPREPQVYT (SEQ ID NO: 74) or a peptide having significant sequence identity and/or three-dimensional structural similarity thereto. In some embodiments these peptides are identical or substantially identical to a portion of an IgG or IgM molecule. U.S. Pat. No. 7,041,796 discloses C3b/C4b Complement Receptor-like molecules and uses thereof to inhibit complement activation. U.S. Pat. No. 6,998,468 discloses anti-C2/C2a inhibitors of complement activation. U.S. Pat. No. 6,676,943 discloses human complement C3-degrading protein from Streptococcus pneumoniae.

### B. Administration

In some embodiments, a provided compound, e.g., a compound of formula I or a pharmaceutically acceptable salt thereof, administered in an amount that inhibits plasma complement activity by an average of no more than 95%, optionally between 50% and 95%, as measured using an alternative pathway assay, a classical pathway assay, or both, may be administered in combination with a second complement inhibitor, e.g., a long-acting compstatin analog (LACA). In some embodiments, a LACA administered in an amount that inhibits plasma complement activity by an average of no more than 95%, optionally between 50% and 95%, as measured using an alternative pathway assay, a classical pathway assay, or both, may be administered in combination with a provided compound. In some embodiments, the assay is a hemolysis assay. In some embodiments, a provided compound administered in an amount effective to reduce the steady state plasma level of C3 by between 30% and 95% on average, e.g., between 50% and 95%, e.g., between 50% and 60%, between 60% and 70%, between 70% and 80%, or between 80% and 90%, on average, may be administered in combination with a LACA. In some embodiments, a provided compound may be administered in amounts that are effective to reduce the steady state plasma level of C3 by more than 95% but still do not achieve a desired efficacy. Combined administration with the LACA allows such efficacy to be achieved. In some embodiments, a provided compound may be administered at between 80% and 100% of its maximum tolerated dose. In some embodiments, combined administration with a LACA allows the use of smaller doses of a provided compound than those required to achieve a desired level of efficacy. In some embodiments, a provided compound may be administered at less than 50%, 60%, 70%, or 80% of its maximum tolerated dose.

In some embodiments, a provided compound and a second therapeutic agent, e.g., a complement inhibitor may be administered once daily, weekly, every 2, 3, or 4 weeks, or even at longer intervals. In some embodiments, a provided compound is administered to a subject once, e.g., as a single injection or as a single infusion over time (e.g., over 5, 10, 15, 20, 30, 40, 50, 60, 90, 120 minutes, or longer), and a second complement inhibitor is administered once a week, twice a week, every three days, thrice a week, every other day, once every 2 weeks, every 3 weeks, once a month, every 6 weeks, once every 2 months, 3 months, 4 months, 5 months, or longer. In some embodiments, a provided compound is administered to a subject twice, e.g., as two injections (e.g., 2, 4, 6, 8, 10, or 12 hours apart) or as two infusions (e.g., 2, 4, 6, 8, 10, or 12 hours apart), and a second complement inhibitor is administered once a week, twice a week, every three days, thrice a week, every other day, once every 2 weeks, every 3 weeks, once a month, every 6 weeks, once every 2 months, 3 months, 4 months, 5 months, or longer. In some embodiments, a provided compound and a second therapeutic agent, e.g., a complement inhibitor may be administered according to a dosing regimen that includes a second complement inhibitor administered once a week, twice a week, every three days, thrice a week, every other day, once every 2 weeks, every 3 weeks, once a month, every 6 weeks, once every 2 months, 3 months, 4 months, 5 months, or longer; and (i) a single administration or an initial administration of a provided compound that is once daily, weekly, every 2, 3, or 4 weeks, or even at longer intervals; followed by (ii) a period of no administration of a provided compound of, e.g., 1, 2, 3, 4, 5, 6, 8, or 10 months, or 1, 2, 3, 4, 5, 6, 7, 8, 9, or 10 years. In some embodiments, a subject is monitored before and/or following treatment for level of C3 expression and/or activity, e.g., as measured using an alternative pathway assay, a classical pathway assay, or both. Suitable assays are known in the art and include, e.g., a hemolysis assay. In some embodiments, a subject is treated (e.g., with a provided compound and/or with a second therapeutic agent, e.g., a complement inhibitor), or is retreated (e.g., with a provided compound and/or with a second therapeutic agent, e.g., a complement inhibitor), if a measured level of C3 expression and/or activity is more than 10%, 20%, 30%, 40%, 50%, 100%, 200%, or more, relative to measured level of C3 expression and/or activity in a control subject.

In some embodiments it may be desirable to administer a provided compound and a second therapeutic agent, e.g., a complement inhibitor according to the same dosing schedule (e.g., once per day, every other day, or once per week), while in other embodiments different dosing schedules may be used. In some embodiments, a provided compound and a second therapeutic agent, e.g., a complement inhibitor are administered subcutaneously. In some embodiments, a provided compound may be administered intravenously.

In some embodiments, a provided compound achieves therapeutically useful levels of complement inhibition when administered as sole complement inhibiting therapy once or twice daily, e.g., subcutaneously. In some embodiments, a provided compound achieves therapeutically useful levels of C3 inhibition when administered as sole C3 inhibiting therapy once or twice daily, e.g., subcutaneously. In some embodiments, a provided compound may be administered in a lower total amount (as measured over a relevant time period such as a month) when administered in combination with a second therapeutic agent, e.g., a complement inhibitor that inhibits C3 expression and/or activity. In some embodiments, a total amount administered may be lower by a factor of at least 1.5, e.g., lower by a factor of between 1.5 and 5, between 5 and 10, or between 10 and 20, over a relevant time period such as a month. In some embodiments, a provided compound may be administered in smaller daily doses as compared with the doses that would be used if a provided compound was administered as sole complement inhibitor therapy or as sole C3 inhibiting therapy. In some embodiments, a provided compound may be administered using a longer dosing interval as compared with the dosing interval that would be used if a provided compound was administered as sole complement inhibitor therapy or as sole C3 inhibiting therapy. For example, in some embodiments, a provided compound that would typically be administered daily to achieve a desired effect may instead be administered every other day, every 3 days, or weekly, to achieve substantially the same effect. In some embodiments, a provided compound may be administered using both lower individual doses and a longer dosing interval when administered in combination with a LACA than when administered as sole complement inhibitor therapy or as sole C3 inhibiting therapy.

Certain LACAs comprising a PEG of about 40 kD demonstrate pharmacological activity when administered subcutaneously at daily doses of 180 mg and 270 mg, with 270 mg/day being particularly effective. In some embodiments, such a LACA, when administered in combination with a provided compound may be administered at a reduced dose, e.g., a dose that is lower by a factor of at least 1.5, e.g., lower by a factor of between 1.5 and 5, between 5 and 10, or between 10 and 20, relative to administration of LACA alone. In some embodiments, for example, the dose may be between about 9 mg/day and about 150 mg/day, e.g., between about 9 mg/day and about 20 mg/day, between about 20 mg/day and about 50 mg/day, between about 50 mg/day and 100 mg/day, between about 100 mg/day and about 150 mg/day, and in at least some embodiments achieves at least equivalent efficacy to a 180 mg/day dose or in some embodiments a 270 mg/day dose. In some embodiments the dose may be between about 150 mg/day and about 200 mg/day, and in at least some embodiments achieves at least equivalent efficacy to a 270 mg/day dose. In some embodiments the dose is 10 mg/day-20 mg/day, 20 mg/day-30 mg/day, 30 mg/day-40 mg/day, 40 mg/day-50 mg/day, 50 mg/day-60 mg/day, 60 mg/day-70 mg/day, 70 mg/day-80 mg/day, 80 mg/day-90 mg/day, 90 mg/day-100 mg/day, 100 mg/day-110 mg/day, 110 mg/day-120 mg/day, 120 mg/day-130 mg/day, 130 mg/day-140 mg/day, 140 mg/day-150 mg/day, 150 mg/day-160 mg/day, 160 mg/day-170 mg/day, 170 mg/day-180 mg/day, 180 mg/day-190 mg/day, or 190 mg/day-200 mg/day. In some embodiments the dose is 200 mg/day-210 mg/day, 210 mg/day-220 mg/day, 220 mg/day-230 mg/day, 230 mg/day-240 mg/day, or 240 mg/day-250 mg/day. In some embodiments the dose of LACA is administered as a single daily dose, e.g., subcutaneously. In some embodiments a dose of LACA is administered as a single weekly dose, e.g., subcutaneously.

In some aspects, a reduced dose of LACA may be administered in a smaller volume and/or at a reduced concentration, when administered in combination with a provided compound, relative to administration of LACA alone. For example, if the dose is reduced by a factor of 10, the volume could also be reduced by a factor of 10 while keeping the concentration the same. Alternately, the concentration could be reduced by a factor of 10 while keeping the volume the same. Alternately both the concentration and volume may be reduced. In certain embodiments the volume of an individual dose is about 0.8 ml or less, e.g., 0.5 ml or less, e.g., between 0.02 ml and 0.5 ml, e.g., 0.1 ml, 0.2 ml, 0.3 ml, 0.4 ml, or 0.5 ml. In certain embodiments the concentration is below about 100 mg/ml. For example, the concentration may be 10 mg/ml-20 mg/ml, 20 mg/ml-30 mg/ml, 30 mg/ml-40 mg/ml, 40 mg/ml-50 mg/ml, 50 mg/ml-60 mg/ml, 60 mg/ml-70 mg/ml, 70 mg/ml-80 mg/ml, 80 mg/ml-90 mg/ml, or 90 mg/ml-100 mg/ml. The volume and concentration can be selected to deliver a desired amount. For example, in an exemplary embodiment a dose of 40 mg is administered in a volume of 0.5 ml at a concentration of 80 mg/ml. In another exemplary embodiment a dose of 60 mg is administered in a volume of 0.6 ml at a concentration of 100 mg/ml. In some embodiments a 28, 29, 30, or 31 gauge needle may be used to administer the LACA, a provided compound, or both.

In some embodiments, a LACA described herein is administered twice weekly or every 3 days or thrice weekly, at a dosage of about 10 mg to about 10 g, e.g., about 10 mg to about 20 mg, e.g., about 20 mg to about 40 mg, e.g., about 40 mg to about 60 mg, e.g., about 60 mg to about 80 mg, e.g., about 80 mg to about 100 mg, e.g., about 100 mg to about 120 mg, e.g., about 120 mg to about 140 mg, e.g., about 140 mg to about 160 mg, e.g., about 160 mg to about 180 mg, e.g., about 180 mg to about 200 mg, e.g., about 200 mg to about 220 mg, e.g., about 220 mg to about 240 mg, e.g., about 240 mg to about 260 mg, e.g., about 260 mg to about 280 mg, e.g., about 280 mg to about 300 mg, e.g., about 300 mg to about 320 mg, e.g., about 320 mg to about 340 mg, e.g., about 340 mg to about 360 mg, e.g., about 360 mg to about 380 mg, e.g., about 380 mg to about 400 mg, e.g., about 400 mg to about 420 mg, e.g., about 420 mg to about 440 mg, e.g., about 440 mg to about 460 mg, e.g., about 460 mg to about 480 mg, e.g., about 480 mg to about 500 mg, e.g., about 500 mg to about 520 mg, e.g., about 520 mg to about 540 mg, e.g., about 540 mg to about 560 mg, e.g., about 560 mg to about 580 mg, e.g., about 580 mg to about 600 mg, e.g., about 100 mg to about 200 mg, e.g., about 545 mg to about 1690 mg, e.g., about 585 mg to about 2510 mg, e.g., about 630 mg to about 930 mg, e.g., about 795 mg to about 885 mg, e.g., about 900 mg to about 1395 mg, e.g., about 990 mg to about 1215 mg, e.g., about 1215 mg to about 1395 mg, e.g., about 2160 mg to about 2520 mg, e.g., about 2520 mg to about 2880 mg, e.g., about 2880 mg to about 3240 mg, e.g., about 3240 mg to about 3600 mg, e.g., about 800 mg to about 1200 mg, e.g., about 1060 mg to about 1100 mg, e.g., about 1070 mg to about 1090 mg, e.g., about 1075 mg to about 1085 mg, e.g., about 1080 mg, e.g., about 1080 mg to about 5040 mg, about 5000 mg-1.0 g, e.g., about 1.0-2.0 g, or more, e.g., up to about 4.0-5.0 g, or up to about 6.0 g, or up to about 10.0 g, e.g., between about 4.0 g and about 6.0 g, e.g., between about 4.5 g and about 5.5 g, e.g., about 5.0 g, e.g., between about 5.0 g and about 7.0 g, e.g., about 5.0 g, about 5.5 g, about 6.0 g, about 6.5 g, or about 7.0 g, e.g., between about 8.0 g and about 10.0 g, e.g., about 8.0 g, about 8.5 g, about 9.0 g, about 9.5 g, or about 10.0 g, e.g., between about 4.0 g and about 10.0 g, e.g., about 5.0 g, about 6.0 g, about 7.0 g, about 8.0 g, about 8.5 g, about 9.0 g, about 9.5 g, or about 10.0 g, for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer.

In some embodiments, a LACA described herein is administered to a subject in need thereof at about 10 mg to about 10 g (e.g., about 10 mg to about 600 mg, about 600 mg to about 1200 mg, about 1250 mg to about 2000 mg, about 2000 mg to about 2500 mg, about 10-20 mg, about 20-40 mg, about 40-60 mg, about 60-80 mg, about 80-100 mg, about 100-120 mg, about 120-140 mg, about 140-160 mg, about 160-180 mg, about 180-200 mg, about 200-220 mg, about 220-240 mg, about 240-260 mg, about 260-280 mg, about 280-300 mg, about 300-320 mg, about 320-340 mg, about 340-360 mg, about 360-380 mg, about 380-400 mg, about 400-420 mg, about 420-440 mg, about 440-460 mg, about 460-480 mg, about 480-500 mg, about 500-520 mg, about 520-540 mg, about 540-560 mg, about 560-580 mg, about 580-600 mg, about 600-620 mg, about 620-640 mg, about 640-660 mg, about 660-680 mg, about 680-700 mg, about 700-720 mg, about 720-740 mg, about 740-760 mg, about 760-780 mg, about 780-800 mg, about 800-820 mg, about 820-840 mg, about 840-860 mg, about 860-880 mg, about 880-900 mg, about 900-920 mg, about 920-940 mg, about 940-960 mg, about 960-980 mg, about 980-1000 mg, about 1000-1020 mg, about 1020-1040 mg, about 1040-1060 mg, about 1060-1080 mg, about 1080-1100 mg, about 1100-1120 mg, about 1120-1140 mg, about 1140-1160 mg, about 1160-1180 mg, about 1180-1200 mg, about 1200-1250 mg, about 1250-1300 mg, about 1300-1350 mg, about 1350-1400 mg, about 1400-1450 mg, about 1450-1500 mg, about 1500-1550 mg, about 1550-1600 mg, about 1600-1650 mg, about 1650-1700 mg, about 1700-about 1750 mg, about 1750-1800 mg, about 1800-1850 mg, about 1850-1900 mg, about 1900-1950 mg, about 1950-2000 mg, about 2000-2050 mg, about 2050-2100 mg, about 2100-2150 mg, about 2150-2200 mg, about 2200-2250 mg, about 2250-2300 mg, about 2300-2350 mg, about 2350-2400 mg, about 2400-2450 mg, about 2450-2500 mg, about 2500-5000 mg, about 5000 mg-1.0 g, about 1.0-2.0 g, or more, e.g., up to about 4.0-5.0 g, or up to about 6.0 g, or up to about 10.0 g, between about 4.0 g and about 6.0 g, e.g., between about 4.5 g and about 5.5 g, e.g., about 5.0 g, between about 5.0 g and about 7.0 g, e.g., about 5.0 g, about 5.5 g, about 6.0 g, about 6.5 g, or about 7.0 g, between about 8.0 g and about 10.0 g, e.g., about 8.0 g, about 8.5 g, about 9.0 g, about 9.5 g, or about 10.0 g, between about 4.0 g and about 10.0 g, e.g., about 5.0 g, about 6.0 g, about 7.0 g, about 8.0 g, about 8.5 g, about 9.0 g, about 9.5 g, or about 10.0 g) or more.

In some embodiments, a LACA described herein is administered as two or more doses. In some embodiments, a first dose (e.g., a loading dose) and a second dose (e.g., a maintenance dose) are administered. In some embodiments, the first dose and the second dose comprise the same amount of the LACA. In some embodiments, the first dose and the second dose comprise different amounts of the LACA.

In some embodiments, the first dose comprises about 10 mg to about 10 g of the LACA (e.g., about 10-20 mg, about 20-40 mg, about 40-60 mg, about 60-80 mg, about 80-100 mg, about 100-120 mg, about 120-140 mg, about 140-160 mg, about 160-180 mg, about 180-200 mg, about 200-220 mg, about 220-240 mg, about 240-260 mg, about 260-280 mg, about 280-300 mg, about 300-320 mg, about 320-340 mg, about 340-360 mg, about 360-380 mg, about 380-400 mg, about 400-420 mg, about 420-440 mg, about 440-460 mg, about 460-480 mg, about 480-500 mg, about 500-520 mg, about 520-540 mg, about 540-560 mg, about 560-580 mg, about 580-600 mg, about 600-620 mg, about 620-640 mg, about 640-660 mg, about 660-680 mg, about 680-700 mg, about 700-720 mg, about 720-740 mg, about 740-760 mg, about 760-780 mg, about 780-800 mg, about 800-820 mg, about 820-840 mg, about 840-860 mg, about 860-880 mg, about 880-900 mg, about 900-920 mg, about 920-940 mg, about 940-960 mg, about 960-980 mg, about 980-1000 mg, about 1000-1020 mg, about 1020-1040 mg, about 1040-1060 mg, about 1060-1080 mg, about 1080-1100 mg, about 1100-1120 mg, about 1120-1140 mg, about 1140-1160 mg, about 1160-1180 mg, about 1180-1200 mg, about 1200-1250 mg, about 1250-1300 mg, about 1300-1350 mg, about 1350-1400 mg, about 1400-1450 mg, about 1450-1500 mg, about 1500-1550 mg, about 1550-1600 mg, about 1600-1650 mg, about 1650-1700 mg, about 1700-about 1750 mg, about 1750-1800 mg, about 1800-1850 mg, about 1850-1900 mg, about 1900-1950 mg, about 1950-2000 mg, about 2000-2050 mg, about 2050-2100 mg, about 2100-2150 mg, about 2150-2200 mg, about 2200-2250 mg, about 2250-2300 mg, about 2300-2350 mg, about 2350-2400 mg, about 2400-2450 mg, about 2450-2500 mg, about 2500-5000 mg, about 5000 mg-1.0 g, about 1.0-2.0 g, or more, e.g., up to about 4.0-5.0 g, or up to about 6.0 g, or up to about 10.0 g, between about 4.0 g and about 6.0 g, e.g., between about 4.5 g and about 5.5 g, e.g., about 5.0 g, between about 5.0 g and about 7.0 g, e.g., about 5.0 g, about 5.5 g, about 6.0 g, about 6.5 g, or about 7.0 g, between about 8.0 g and about 10.0 g, e.g., about 8.0 g, about 8.5 g, about 9.0 g, about 9.5 g, or about 10.0 g, between about 4.0 g and about 10.0 g, e.g., about 5.0 g, about 6.0 g, about 7.0 g, about 8.0 g, about 8.5 g, about 9.0 g, about 9.5 g, or about 10.0 g) and the second dose comprises about 10 mg to about 10 g of the LACA (e.g., about 10-20 mg, about 20-40 mg, about 40-60 mg, about 60-80 mg, about 80-100 mg, about 100-120 mg, about 120-140 mg, about 140-160 mg, about 160-180 mg, about 180-200 mg, about 200-220 mg, about 220-240 mg, about 240-260 mg, about 260-280 mg, about 280-300 mg, about 300-320 mg, about 320-340 mg, about 340-360 mg, about 360-380 mg, about 380-400 mg, about 400-420 mg, about 420-440 mg, about 440-460 mg, about 460-480 mg, about 480-500 mg, about 500-520 mg, about 520-540 mg, about 540-560 mg, about 560-580 mg, about 580-600 mg, about 600-620 mg, about 620-640 mg, about 640-660 mg, about 660-680 mg, about 680-700 mg, about 700-720 mg, about 720-740 mg, about 740-760 mg, about 760-780 mg, about 780-800 mg, about 800-820 mg, about 820-840 mg, about 840-860 mg, about 860-880 mg, about 880-900 mg, about 900-920 mg, about 920-940 mg, about 940-960 mg, about 960-980 mg, about 980-1000 mg, about 1000-1020 mg, about 1020-1040 mg, about 1040-1060 mg, about 1060-1080 mg, about 1080-1100 mg, about 1100-1120 mg, about 1120-1140 mg, about 1140-1160 mg, about 1160-1180 mg, about 1180-1200 mg, about 1200-1250 mg, about 1250-1300 mg, about 1300-1350 mg, about 1350-1400 mg, about 1400-1450 mg, about 1450-1500 mg, about 1500-1550 mg, about 1550-1600 mg, about 1600-1650 mg, about 1650-1700 mg, about 1700-about 1750 mg, about 1750-1800 mg, about 1800-1850 mg, about 1850-1900 mg, about 1900-1950 mg, about 1950-2000 mg, about 2000-2050 mg, about 2050-2100 mg, about 2100-2150 mg, about 2150-2200 mg, about 2200-2250 mg, about 2250-2300 mg, about 2300-2350 mg, about 2350-2400 mg, about 2400-2450 mg, about 2450-2500 mg, about 2500-5000 mg, about 5000 mg-1.0 g, about 1.0-2.0 g, or more, e.g., up to about 4.0-5.0 g, or up to about 6.0 g, or up to about 10.0 g, between about 4.0 g and about 6.0 g, e.g., between about 4.5 g and about 5.5 g, e.g., about 5.0 g, between about 5.0 g and about 7.0 g, e.g., about 5.0 g, about 5.5 g, about 6.0 g, about 6.5 g, or about 7.0 g, between about 8.0 g and about 10.0 g, e.g., about 8.0 g, about 8.5 g, about 9.0 g, about 9.5 g, or about 10.0 g, between about 4.0 g and about 10.0 g, e.g., about 5.0 g, about 6.0 g, about 7.0 g, about 8.0 g, about 8.5 g, about 9.0 g, about 9.5 g, or about 10.0 g).

In some embodiments, a provided compound is administered to a subject in combination with a LACA, such that the LACA and/or a provided compound is administered less frequently and/or at a lower dosage, relative to administration of a LACA alone or relative to administration of the provided compound alone.

In some embodiments, a provided compound is administered to a subject in combination with a LACA, e.g., a LACA comprising a PEG of about 40 kD, such that the LACA is administered once a week, twice a week, every three days, thrice a week, every other day, once every 2 weeks, every 3 weeks, once a month, every 6 weeks, once every 2 months, 3 months, 4 months, 5 months, or longer, at a dosage described herein (for e.g., at a dosage of about 10 mg to about 10 g, about 800 mg to about 1200 mg, e.g., about 1060 mg to about 1100 mg, e.g., about 1070 mg to about 1090 mg, e.g., about 1075 mg to about 1085 mg, e.g., about 1080 mg, e.g., about 1.0-2.0 g, or more, e.g., up to about 4.0-5.0 g, or up to about 6.0 g, or up to about 10.0 g, between about 4.0 g and about 6.0 g, e.g., between about 4.5 g and about 5.5 g, e.g., about 5.0 g, between about 5.0 g and about 7.0 g, e.g., about 5.0 g, about 5.5 g, about 6.0 g, about 6.5 g, or about 7.0 g, between about 8.0 g and about 10.0 g, e.g., about 8.0 g, about 8.5 g, about 9.0 g, about 9.5 g, or about 10.0 g, between about 4.0 g and about 10.0 g, e.g., about 5.0 g, about 6.0 g, about 7.0 g, about 8.0 g, about 8.5 g, about 9.0 g, about 9.5 g, or about 10.0 g).

In some embodiments, a provided compound is administered to a subject once, e.g., as a single injection or as a single infusion over time (e.g., over 5, 10, 15, 20, 30, 40, 50, 60, 90, 120 minutes, or longer), in combination with a LACA, e.g., a LACA of comprising a PEG of about 40 kD, such that the LACA is administered once a week, twice a week, every three days, thrice a week, every other day, once every 2 weeks, once every 3 weeks, once a month, once every 6 weeks, once every 2 months, 3 months, 4 months, 5 months, or longer, at a dosage described herein (for e.g., at a dosage of about 10 mg to about 10 g, about 800 mg to about 1200 mg, e.g., about 1060 mg to about 1100 mg, e.g., about 1070 mg to about 1090 mg, e.g., about 1075 mg to about 1085 mg, e.g., about 1080 mg, e.g., about 1.0-2.0 g, or more, e.g., up to about 4.0-5.0 g, or up to about 6.0 g, or up to about 10.0 g, between about 4.0 g and about 6.0 g, e.g., between about 4.5 g and about 5.5 g, e.g., about 5.0 g, between about 5.0 g and about 7.0 g, e.g., about 5.0 g, about 5.5 g, about 6.0 g, about 6.5 g, or about 7.0 g, between about 8.0 g and about 10.0 g, e.g., about 8.0 g, about 8.5 g, about 9.0 g, about 9.5 g, or about 10.0 g, between about 4.0 g and about 10.0 g, e.g., about 5.0 g, about 6.0 g, about 7.0 g, about 8.0 g, about 8.5 g, about 9.0 g, about 9.5 g, or about 10.0 g).

In some embodiments, a provided compound is administered to a subject twice, e.g., as two injections (e.g., 2, 4, 6, 8, 10, or 12 hours apart) or as two infusions (e.g., 2, 4, 6, 8, 10, or 12 hours apart), in combination with a LACA, e.g., a LACA comprising a PEG of about 40 kD, such that the LACA is administered once a week, twice a week, every three days, thrice a week, every other day, once every 2 weeks, once every 3 weeks, once a month, once every 6 weeks, once every 2 months, 3 months, 4 months, 5 months, or longer, at a dosage described herein (for e.g., at a dosage of about 10 mg to about 10 g, about 800 mg to about 1200 mg, e.g., about 1060 mg to about 1100 mg, e.g., about 1070 mg to about 1090 mg, e.g., about 1075 mg to about 1085 mg, e.g., about 1080 mg, e.g., about 1.0-2.0 g, or more, e.g., up to about 4.0-5.0 g, or up to about 6.0 g, or up to about 10.0 g, between about 4.0 g and about 6.0 g, e.g., between about 4.5 g and about 5.5 g, e.g., about 5.0 g, between about 5.0 g and about 7.0 g, e.g., about 5.0 g, about 5.5 g, about 6.0 g, about 6.5 g, or about 7.0 g, between about 8.0 g and about 10.0 g, e.g., about 8.0 g, about 8.5 g, about 9.0 g, about 9.5 g, or about 10.0 g, between about 4.0 g and about 10.0 g, e.g., about 5.0 g, about 6.0 g, about 7.0 g, about 8.0 g, about 8.5 g, about 9.0 g, about 9.5 g, or about 10.0 g).

While doses of 250 mg/day or less are of particular interest for administration, e.g., SC administration, of a LACA in combination with a provided compound that inhibits C3 activity, the present disclosure also contemplates administering doses of more than 250 mg/day in combination with a provided compound that inhibits C3 activity, e.g., doses of 250 mg/day-300 mg/day, 300 mg/day-400 mg/day, or 400 mg/day-500 mg/day. In certain embodiments such a dose may be administered weekly, twice a week, or every 3 days. In certain embodiments, LACA doses in combination with a provided compound that inhibits C3 activity may be administered twice weekly or every 3 days, at a dosage of about 10 mg to about 10 g, about 800 mg to about 1200 mg, e.g., about 1060 mg to about 1100 mg, e.g., about 1070 mg to about 1090 mg, e.g., about 1075 mg to about 1085 mg, e.g., about 1080 mg.

While the present disclosure particularly contemplates embodiments in which a LACA having a terminal half-life of at least 2, 3, 4, or more days when administered IV or SC to a primate, e.g., LACAs comprising a clearance reducing moiety as described herein, is administered in combination with a compound that inhibits C3 activity, it is contemplated in certain embodiments that combined administration with such a provided compound may also be useful for compstatin analogs that have shorter half-lives and/or that lack a clearance reducing moiety. Such compstatin analogs may be administered in 1 or 2 doses per day.

In some embodiments efficacy of a particular agent or combination of agents may be measured by lactate dehydrogenase (LDH) level in a patient suffering from a complement-mediated hemolytic disorder such as PNH. As will be appreciated by those of ordinary skill in the art, complement-mediated hemolysis results in release of LDH, which can result in an abnormally elevated plasma LDH level. Thus in some aspects, efficacy of a complement inhibitor may be evidenced in a subject suffering from a complement-mediated hemolytic disorder by a decrease in plasma LDH level, e.g., to within normal limits. Other indicators of efficacy in a subject suffering from a complement-mediated hemolytic disorder may include, e.g., a reduction in reticulocyte count in a subject who has an elevated reticulocyte count (e.g., normalization of reticulocyte count), a reduced need for transfusions, an increased hemoglobin level, stabilization of hemoglobin level without need for transfusions in a subject who had required multiple transfusions in the previous year. In some embodiments efficacy may be measured by a classical or alternative pathway complement assay, which may be a hemolysis assay.

In some embodiments a composition as described herein, and e.g., a composition comprising a LACA described herein, is administered using a device that delivers a dose of a pharmaceutical composition by injection, in some embodiments in an at least partly automated fashion upon activation. Such a device is referred to in the art as a “pen” or “autoinjector”, and these terms are used interchangeably herein. In general, a pen or autoinjector allows for injecting a dose of pharmaceutical composition contained in a cartridge, reservoir, or syringe through an automatically or manually inserted hypodermic needle(s) or through a high velocity jet. It may be designed for administration of a single dose or multiple doses.

In some embodiments, such a pen or autoinjector is utilized for intramuscular and/or subcutaneous injection. In accordance with the present disclosure, a pen or other autoinjector may be particularly useful for embodiments that utilize subcutaneous injection. Pens are typically devices that contain (or can be loaded with) a medication in a self-contained cartridge or reservoir and to which a needle can be attached.

In some embodiments, such injection is achieved by use of a pen (e.g., that may have been pre-loaded with an appropriate dose or volume). Pens can be durable (and reusable) or disposable. A durable pen typically uses a replaceable cartridge, which is disposed of when empty, and a new one is inserted in the pen. A disposable pen typically comes pre-filled with a medication in a cartridge or reservoir. When the cartridge or reservoir is empty, the pen can be discarded. The cartridge or reservoir may contain a single dose or multiple doses. To use a pen, a needle can be attached to the pen and inserted into the skin. Typically, a button can be pushed to administer a dose though in some embodiments other activation methods may be used. In some embodiments, an autoinjector may comprise a spring-loaded syringe, though one of ordinary skill in the art will appreciate that a variety of technologies are available to afford automatic administration. In some embodiments, by pressing a button or otherwise activating the device, the needle can be automatically inserted, and the medication can be delivered. In some embodiments, an autoinjector may be designed to insert the needle automatically and/or accurately to a desired depth in the subcutaneous tissue. A pen or autoinjector may comprise means such as a dial that allows a user to select or adjust a dose or injection depth.

In some embodiments, a composition as described herein, e.g., a LACA described herein, is administered using a device comprising a dual chamber syringe. Dry drug (e.g., lyophilized) is contained in one chamber. The second chamber contains a suitable pharmaceutically acceptable carrier. In order to use the device, the drug is first reconstituted by mixing the contents of the chambers. This can be accomplished in various ways, as is known in the art. In some embodiments, pushing the plunger causes the contents of the chambers to mix, e.g., by transferring the carrier into the chamber containing the lyophilized drug.

Thus a variety of drug delivery devices comprising a composition as described herein (e.g., a LACA described herein) may be provided e.g., prefilled syringes, dual chamber syringes, durable and/or disposable pens, and cartridges suitable for use with a pen. Such devices may contain one or more doses (e.g., one or more of any of the dose amounts described herein).

In certain embodiments a LACA may be administered, e.g., subcutaneously, using a drug delivery device (sometimes referred to simply as a “delivery device”) that comprises a pump to introduce a liquid composition comprising the LACA into the subject's body. As will be appreciated, a pump may be any device that moves fluids by mechanical action as opposed to a conventional manually actuated syringe characterized in that the individual administering the medication (e.g., a health care provider or a subject who self-administers the medication) must directly depress a plunger into a barrel containing medication in order to effect the injection. It will be appreciated that a pump may be powered electrically or mechanically, e.g., as described herein. In some aspects, a delivery device comprising a pump may allow for convenient administration of doses according to a dosing regimen described herein.

In certain embodiments, the delivery device is portable. A portable device, also referred to as an “ambulatory” device, can be sufficiently light in weight and have appropriate dimensions so as to permit the subject to move about freely while the device is in use. In certain embodiments, such device does not require attachment to a pole or power outlet. In some embodiments a portable delivery device may be attached to a belt or shoulder strap or worn in a case that may be attached to a belt or shoulder strap, or may be placed in a pocket of a garment.

One of ordinary skill in the art appreciates that a pump may operate in any of a variety of ways and may utilize a variety of energy sources, e.g., disposable or rechargeable batteries, alternating current power supply (e.g., via a wall socket in a building), compressed gas, or energy stored in a compressed spring or in a stretched expandable resilient chamber. A device in which fluid is held in a stretchable balloon reservoir, and pressure from the elastic walls of the balloon reservoir drives fluid delivery may be referred to as an “elastomeric infusion pump”.

In some embodiments, a delivery device comprises a pump and a syringe containing a liquid to be administered and removably associated with the device, and a driving unit, which may be electronically controlled by a controller, arranged to make the plunger of the syringe slide so as to cause infusion of the liquid directly or via flexible tubing through a piercing member such as a needle or cannula that is introduced into the subject's body under the skin. For example, in some embodiments a pump may comprise a motor that turns a screw that pushes the plunger on a syringe that contains the liquid. Pushing of the plunger causes liquid to be expelled from the syringe and introduced into the subject's body via an attached piercing member. Exemplary pumps are described in, e.g., U.S. Pat. Nos. 6,447,487; 6,592,551; 6,645,177; 8,187,228; US Patent Application Publication Nos. 20020123740, 20030229311, 20060184123, 20070100281, 20090123309, 20150038906. The Crono PID (NDC No.: 8423.2000.02), Crono S-PID30, and Crono S-PID 50 (NDC No.: 8423.2000.04) (Canè s.r.l. Medical Technology (Rivoli, Italy)), and the T34™ Ambulatory Syringe Pump and the T60™ Ambulatory Syringe Pump (CME Medical, Blackpool, UK) are exemplary portable syringe infusion pumps that may be used in certain embodiments.

In some embodiments the pump may be electronically programmable or controlled. In some embodiments the pump is not electronically programmable or controlled.

In some embodiments a pump uses electricity as a source of power. In some embodiments a pump does not use electricity as a source of power. Such a pump may, for example, use a compressed spring or compressed gas as an energy source.

In some embodiments the pump is a constant-pressure pump that applies a constant pressure to depress the barrel of a syringe containing the liquid to be administered. An example of a constant-pressure pump is the Freedom60® infusion system (RMS Medical Products, Chester, NY). In some embodiments a FreedomEdge® infusion system (RMS Medical Products) may be used, e.g., with a syringe capable of holding up to 20 ml or a syringe capable of holding up to 30 ml. Another example of a constant pressure device is the SCIg60 syringe pump (EMED Technologies, El Dorado Hills, CA). In some embodiments a valve may control the flow rate of the liquid. In some embodiments tubing connected to the syringe may control the flow rate of the liquid, e.g., as described in US Patent Application Nos. 20150374911 and/or 20160256625. In some embodiments a delivery rate of between 0.5 ml/minute and 1 ml/minute may be used.

In some embodiments the liquid to be administered is contained in a pressurized chamber prior to administration. In some embodiments the liquid is contained in a resilient, expandable container portion such as a bladder or balloon prior to delivery. The expandable container portion may be made of or comprise an inner lining of compatible medical grade butyl, silicone or other material suitable for holding the liquid. The container portion expands upon filling with liquid (e.g., with a unit dose of the compound to be administered), so as to exert pressure on the liquid. One of ordinary skill in the art appreciates that the container portion may be filled in a variety of ways. In some embodiments filling of the expandable container portion may be accomplished manually, e.g., using a manually actuated syringe, or may be performed using a filling apparatus. After the device is attached to the subject's skin, a piercing member such as a needle or cannula, which may be spring loaded, may automatically or following additional activation, such as by pressing a button, emerge from the device's housing and pierce the skin. Subsequently, either automatically or following additional activation, such as by pressing a button, pressure forces the liquid out of the chamber or container and into the subject's body via the needle or cannula. Exemplary devices are described in US Patent Application Pub. Nos. 20130018326, and/or 20150217058.

In some embodiments the delivery device is an “on-body delivery device”, which term refers to a delivery device comprising a chamber or other container portion for holding a liquid to be administered to a subject, wherein the device can deliver the liquid while attached directly to the subject's skin without the need for a separate support or external reservoir and, typically, permits the subject to be mobile during delivery. The chamber for holding the liquid may be contained in a housing. Typically, an on-body delivery device is affixed to the subject's skin using an adhesive. The device is affixed sufficiently strongly so that the device is self-supporting. The device may be provided with an adhesive layer, e.g., on the outer surface of the housing, for use to secure the device directly to the skin. The adhesive layer may surround the portion of the device from which a piercing member such as a needle or cannula projects so as to provide a seal around the penetrated skin. In some embodiments an on-body delivery device is available from Sensile Medical AG (Hagendorf, Switzerland). For example, devices known as SenseInfuse, SensePatch, or Senseflex, may be used. In some embodiments an on-body delivery device is available from Enable Injections, Inc. (Cincinnati, OH). In some embodiments the device that comprises a resilient, expandable container portion such as a bladder or balloon to expel the liquid is an on-body delivery device. In some embodiments the device, e.g., an on-body delivery device, is configured such that the piercing member, e.g., needle, is not visible to the user prior to or during use of the device. In some embodiments, the piercing member, e.g., needle, may retract when delivery of the liquid is complete or when the device is removed from the skin. It will be appreciated that a piercing member, e.g., a needle, for use with a delivery device described herein may have any suitable gauge or inner diameter, e.g., such gauge or inner diameters as described elsewhere herein.

In some embodiments, a delivery device comprises a housing into which a vial, cartridge, or syringe containing a liquid (e.g., a liquid comprising a LACA) may be inserted. The liquid is administered upon activation of the device. In some embodiments the liquid is transferred to a chamber of the device prior to administration. In some embodiments a delivery device is reusable, e.g., it can be re-filled or supplied with a new vial, cartridge, or syringe following administration of the contents.

In some embodiments a delivery device is a single use device, i.e., the device is designed to be used to administer a single dose or for use in a single administration session. For example, a device may be designed to be affixed to the skin of a subject, activated to administer a dose, removed, and then recycled or discarded rather than used to administer one or more additional doses.

In some embodiments a delivery device that allows delivery of a liquid into two or more sites may be used. In some embodiments the number of sites is between 1 and 5. In some embodiments the number of sites is greater than 5, e.g., between 6 and 10. Delivery to the two or more sites may be simultaneous or sequential. The device may comprise a pair of syringes, each arranged to be connected to one of the sites and coupled to a body that houses a driving system of the device. Exemplary devices are described in WO2011154928 and US Patent Application Publication No. 20120143133. In some embodiments a multi-needle infusion set may be used. In some embodiments a multi-needle infusion set comprises a flexible tube that communicates at one end with a chamber (which term is used interchangeably with “reservoir”) containing the liquid (e.g., a syringe) while the other end bifurcates into multiple tubes each having a needle at the end. The Neria™ multi infusion sets (Unomedical A/S, Osted, Denmark) are exemplary multi-needle infusion sets.

In some embodiments a delivery device may collect data regarding use of the device. Such data may comprise, for example, the date and time at which the device was used, delivery parameters such as the volume administered, the duration of administration, whether any problems occurred during administration, etc. The data may be stored on a computer-readable medium physically associated with the device and/or may be transmitted to a remote location, e.g., a remote server, where it may be stored, analyzed, or further transmitted for storage or analysis. The device may comprise one or more processors, sensors, software programs, and appropriate connectivity that allow data to be exchanged between the device and other products and systems. Data may be transferred via radio-frequency identification (RFID), bar-code/QR-code scanning, cellular, Bluetooth low energy (BTLE), physical wire, or a combination thereof. The data may be transmitted over any suitable network, e.g., the Internet. The data may be analyzed and/or stored in the Cloud. In some embodiments the device comprises an active or passive RFID tag or chip, hereinafter referred to as an “RFID tag”. The RFID tag may contain data that identifies the device. The RFID tag may be an active tag or chip that signals usage-related information such as activation of the device and/or completion of an administration of a dose. In some embodiments data acquired from a particular device may be made available to one or more entities or individuals, such as health care providers or caregivers of the subject. Such entities or individuals may additionally or alternately be automatically notified of the occurrence or non-occurrence of specified events. For example, if a dose is not administered on a day on which such administration is to take place according to the dosing schedule, or if the device is deployed on a day when administration is not supposed to take place according to the dosing schedule, one or more health care providers or caregivers of the subject may be notified. Once notified, an entity or individual may take appropriate action, such as contacting the subject. In some embodiments a monitoring system automatically attempts to contact the subject, e.g., by phone or text message, if a dose is not administered as scheduled.

In some embodiments a delivery system may comprise a delivery device and a remote control device. The remote control device may, for example, allow programming of the delivery device and/or may be used to activate the delivery device to start delivery of the fluid or to cause the delivery device to cease delivery of the fluid.

In some embodiments, the present disclosure contemplates providing to a subject (e.g., by mail or arranged pickup or other regular mode of delivery) a set of devices as described herein that together provide a supply of active agent (e.g., LACA) sufficient to last for a predetermined period of time (e.g., one week, two weeks, three weeks, four weeks, etc.). In some embodiments, such a set is sent to the patient's residence on a regular basis (e.g., every week, two weeks, three weeks, four weeks, etc.) with a timing selected such that the patient does not run out. In some embodiments, a composition (e.g., comprising a LACA) may be contained in a container (e.g., a vial) or in any of the herein-mentioned drug delivery devices or packs. In some embodiments the supply is sufficient to last for between 4 and 12 weeks, between 12 and 26 weeks, or more.

Those skilled in the art, reading the present disclosure, will appreciate that, in accordance with standard practice in the field, a container containing a particular volume, as described herein may include an additional volume sufficient to permit the designated particular volume (e.g., unit dose) to be withdrawn from the container for administration.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

The disclosure is further illustrated by the following examples. The examples are provided for illustrative purposes only. They are not to be construed as limiting the scope or content of the disclosure in any way.

### VI. Manufacturing

Various technologies, e.g., synthetic chemistry technologies, formulation technologies, assays, etc. can be utilized to prepare, characterization, and assess compounds, compositions and methods described herein in accordance with the present disclosure. Certain technologies are described below and in the Examples.

In some embodiments, the present disclosure provides a method for preparing a compound of formula I or a salt thereof, comprising removing one or more protection groups from a compound of formula M-I:

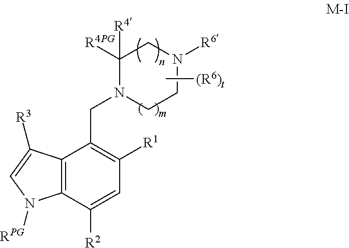

or a salt thereof to provide a compound of formula I or a salt thereof, wherein:
R^{PG} is an amino protecting group,
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group; and
each other variable is independently as described herein (e.g., see, for a compound of formula I or a pharmaceutically acceptable salt thereof).

In some embodiments, R^{PG} is —C(O)R, wherein R is optionally substituted C₁₋₆ aliphatic. In some embodiments, R is optionally substituted C₁-C₆ alkyl. In some embodiments, R is t-butyl.

In some embodiments, R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl. In some embodiments, R^{4PG} is phenyl substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl. In some embodiments, R^{4PG} is

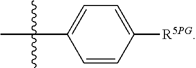

In some embodiments, R^{5PG} is protected carboxyl. In some embodiments, R^{5PG} is —C(O)R^{8PG}, wherein R^{8PG} is optionally substituted C₁₋₆ aliphatic. In some embodiments, R^{8PG} is optionally substituted C₁-C₆ alkyl. In some embodiments, R^{8PG} is methyl. In some embodiments, R^{8PG} is t-butyl.

In some embodiments, a compound of formula I is

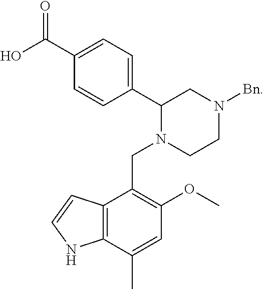 [SMILES: COc1cc(C)c2[nH]ccc2c1CN1CCN(Cc2ccccc2)CC1c1ccc(C(=O)O)cc1]

In some embodiments, a compound of formula M-I is

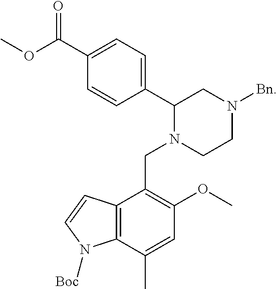 [SMILES: COC(=O)c1ccc(C2CN(Cc3ccccc3)CCN2Cc2c(OC)cc(C)c3c2ccn3C(=O)OC(C)(C)C)cc1]

In some embodiments, a compound of formula I is

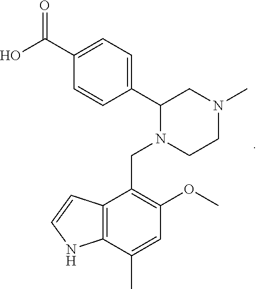 [SMILES: COc1cc(C)c2[nH]ccc2c1CN1CCN(C)CC1c1ccc(C(=O)O)cc1]

In some embodiments, a compound of formula M-I is

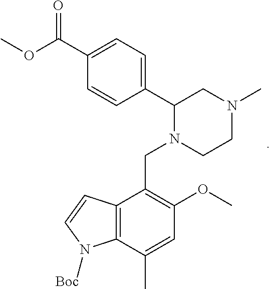

In some embodiments, a compound of formula I is

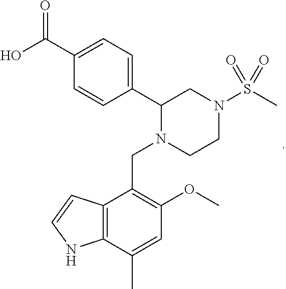 [SMILES: COc1cc(C)c2[nH]ccc2c1CN1CCN(S(C)(=O)=O)CC1c1ccc(C(=O)O)cc1]

In some embodiments, a compound of formula M-I is

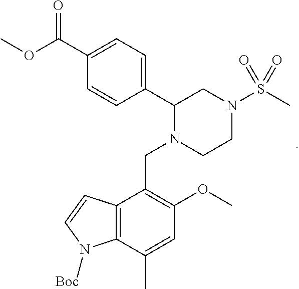

In some embodiments, a compound of formula I is

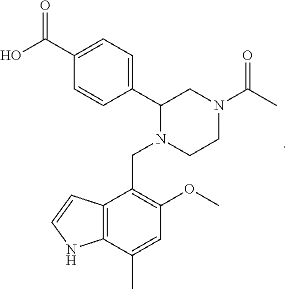 [SMILES: COc1cc(C)c2[nH]ccc2c1CN1CCN(C(C)=O)CC1c1ccc(C(=O)O)cc1]

In some embodiments, a compound of formula M-I is

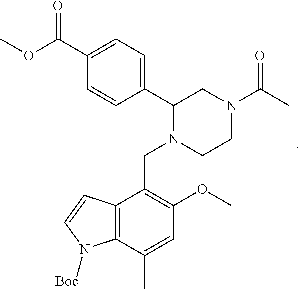 [SMILES: COC(=O)c1ccc(C2CN(C(C)=O)CCN2Cc2c(OC)cc(C)c3c2ccn3C(=O)OC(C)(C)C)cc1]

In some embodiments, a compound of formula I is

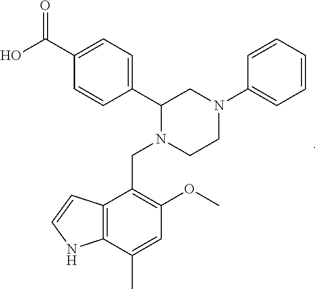 [SMILES: COc1cc(C)c2[nH]ccc2c1CN1CCN(c2ccccc2)CC1c1ccc(C(=O)O)cc1]

In some embodiments, a compound of formula M-I is

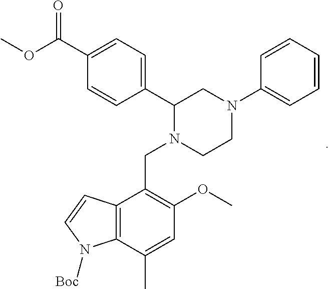 [SMILES: COC(=O)c1ccc(C2CN(c3ccccc3)CCN2Cc2c(OC)cc(C)c3c2ccn3C(=O)OC(C)(C)C)cc1]

In some embodiments, a compound of formula I is

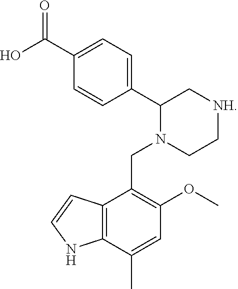 [SMILES: COc1cc(C)c2[nH]ccc2c1CN1CCNCC1c1ccc(C(=O)O)cc1]

In some embodiments, a compound of formula M-I is

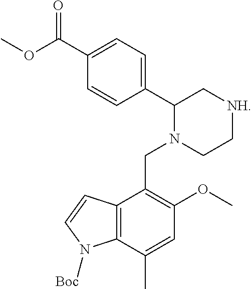

In some embodiments, a compound of formula I is

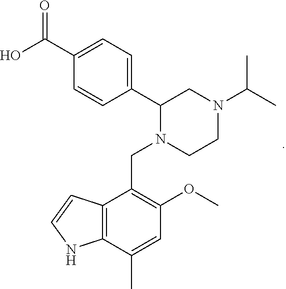 [SMILES: COc1cc(C)c2[nH]ccc2c1CN1CCN(C(C)C)CC1c1ccc(C(=O)O)cc1]

In some embodiments, a compound of formula M-I is

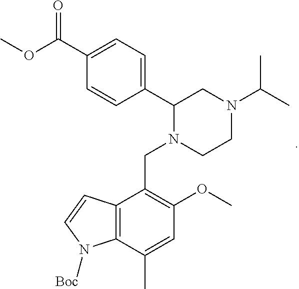 [SMILES: COC(=O)c1ccc(C2CN(C(C)C)CCN2Cc2c(OC)cc(C)c3c2ccn3C(=O)OC(C)(C)C)cc1]

In some embodiments, a compound of formula I is

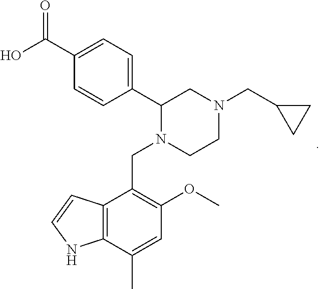 [SMILES: COc1cc(C)c2[nH]ccc2c1CN1CCN(CC2CC2)CC1c1ccc(C(=O)O)cc1]

In some embodiments, a compound of formula M-I is

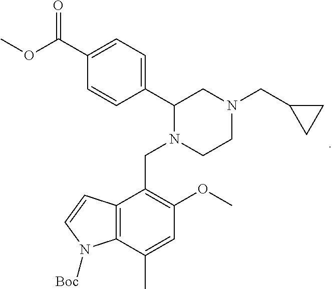 [SMILES: COC(=O)c1ccc(C2CN(CC3CC3)CCN2Cc2c(OC)cc(C)c3c2ccn3C(=O)OC(C)(C)C)cc1]

In one embodiment, a compound of formula I is

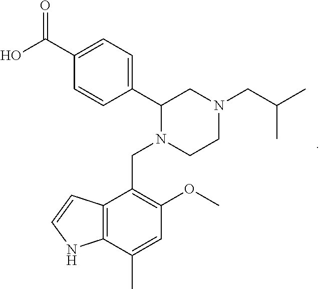 [SMILES: COc1cc(C)c2[nH]ccc2c1CN1CCN(CC(C)C)CC1c1ccc(C(=O)O)cc1]

In some embodiments, a compound of formula M-I is

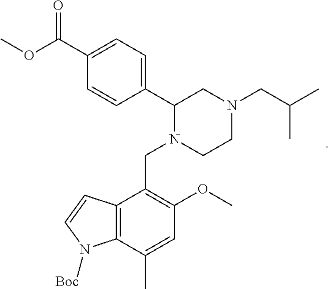 [SMILES: COC(=O)c1ccc(C2CN(CC(C)C)CCN2Cc2c(OC)cc(C)c3c2ccn3C(=O)OC(C)(C)C)cc1]

In some embodiments, a compound of formula I is

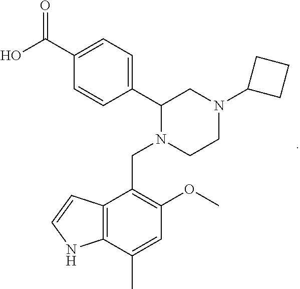 [SMILES: COc1cc(C)c2[nH]ccc2c1CN1CCN(C2CCC2)CC1c1ccc(C(=O)O)cc1]

In some embodiments, a compound of formula M-I is

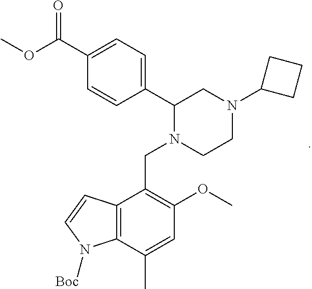 [SMILES: COC(=O)c1ccc(C2CN(C3CCC3)CCN2Cc2c(OC)cc(C)c3c2ccn3C(=O)OC(C)(C)C)cc1]

In some embodiments, a compound of formula I is

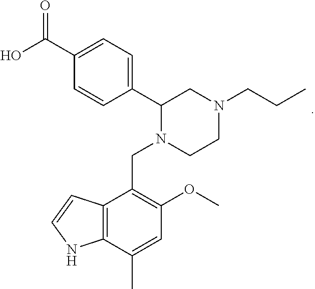 [SMILES: CCCN1CCN(Cc2c(OC)cc(C)c3[nH]ccc23)C(c2ccc(C(=O)O)cc2)C1]

In some embodiments, a compound of formula M-I is

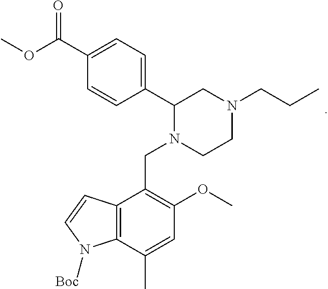 [SMILES: CCCN1CCN(Cc2c(OC)cc(C)c3c2ccn3C(=O)OC(C)(C)C)C(c2ccc(C(=O)OC)cc2)C1]

In some embodiments, a compound of formula I is

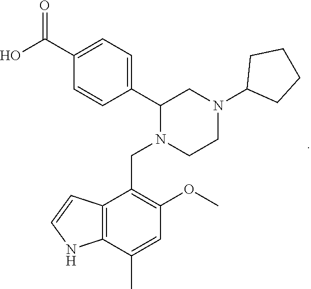 [SMILES: COc1cc(C)c2[nH]ccc2c1CN1CCN(C2CCCC2)CC1c1ccc(C(=O)O)cc1]

In some embodiments, a compound of formula M-I is

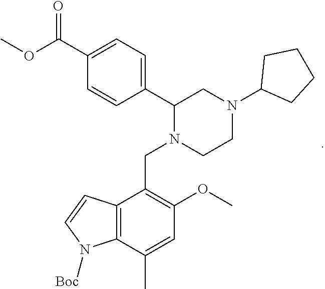 [SMILES: COC(=O)c1ccc(C2CN(C3CCCC3)CCN2Cc2c(OC)cc(C)c3c2ccn3C(=O)OC(C)(C)C)cc1]

In some embodiments, a compound of formula I is

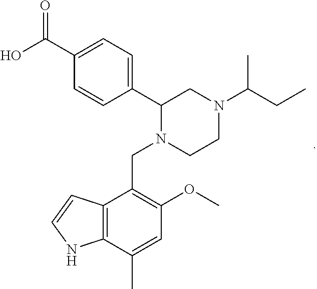 [SMILES: CCC(C)N1CCN(Cc2c(OC)cc(C)c3[nH]ccc23)C(c2ccc(C(=O)O)cc2)C1]

In some embodiments, a compound of formula M-I is

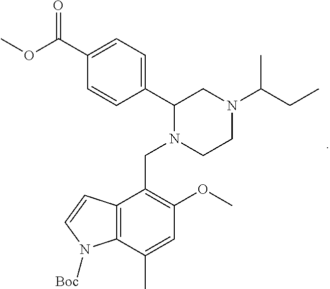 [SMILES: CCC(C)N1CCN(Cc2c(OC)cc(C)c3c2ccn3C(=O)OC(C)(C)C)C(c2ccc(C(=O)OC)cc2)C1]

In some embodiments, a compound of formula I is

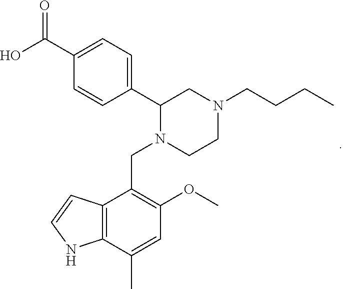 [SMILES: CCCCN1CCN(Cc2c(OC)cc(C)c3[nH]ccc23)C(c2ccc(C(=O)O)cc2)C1]

In some embodiments, a compound of formula M-I is

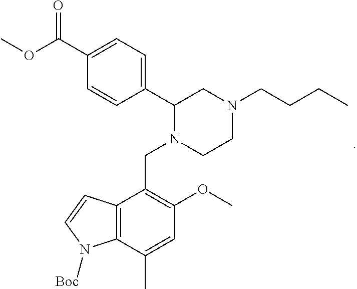 [SMILES: CCCCN1CCN(Cc2c(OC)cc(C)c3c2ccn3C(=O)OC(C)(C)C)C(c2ccc(C(=O)OC)cc2)C1]

In some embodiments, a compound of formula I is

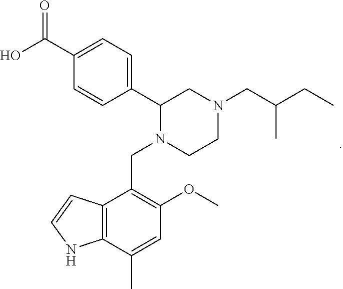 [SMILES: CCC(C)CN1CCN(Cc2c(OC)cc(C)c3[nH]ccc23)C(c2ccc(C(=O)O)cc2)C1]

In some embodiments, a compound of formula M-I is

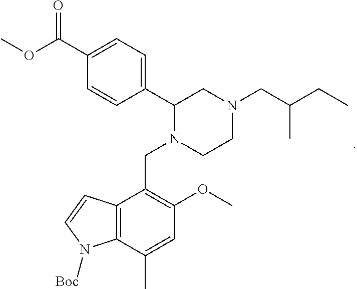 [SMILES: CCC(C)CN1CCN(Cc2c(OC)cc(C)c3c2ccn3C(=O)OC(C)(C)C)C(c2ccc(C(=O)OC)cc2)C1]

In some embodiments, a compound of formula I is

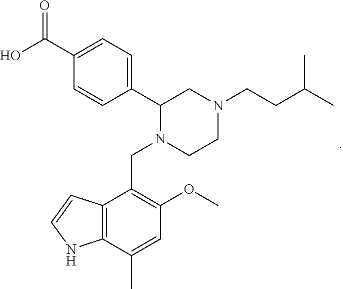 [SMILES: COc1cc(C)c2[nH]ccc2c1CN1CCN(CCC(C)C)CC1c1ccc(C(=O)O)cc1]

In some embodiments, a compound of formula M-I is

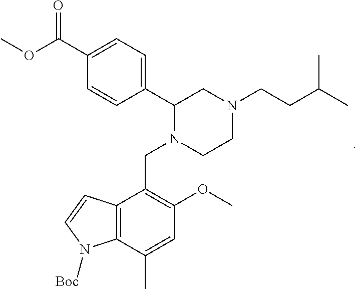 [SMILES: COC(=O)c1ccc(C2CN(CCC(C)C)CCN2Cc2c(OC)cc(C)c3c2ccn3C(=O)OC(C)(C)C)cc1]

In some embodiments, a compound of formula I is

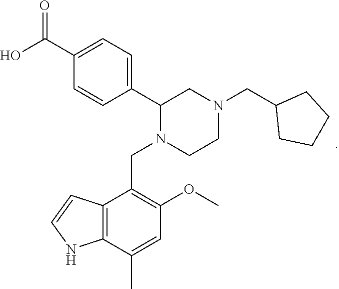 [SMILES: COc1cc(C)c2[nH]ccc2c1CN1CCN(CC2CCCC2)CC1c1ccc(C(=O)O)cc1]

In some embodiments, a compound of formula M-I is

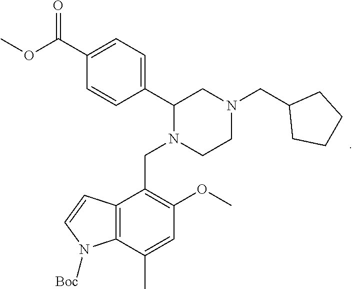 [SMILES: COC(=O)c1ccc(C2CN(CC3CCCC3)CCN2Cc2c(OC)cc(C)c3c2ccn3C(=O)OC(C)(C)C)cc1]

In some embodiments, a compound of formula I is

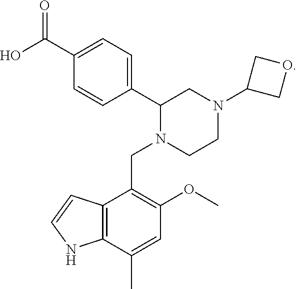 [SMILES: COc1cc(C)c2[nH]ccc2c1CN1CCN(C2COC2)CC1c1ccc(C(=O)O)cc1]

In some embodiments, a compound of formula M-I is

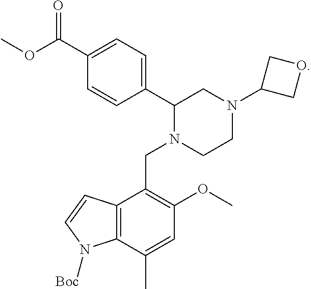 [SMILES: COC(=O)c1ccc(C2CN(C3COC3)CCN2Cc2c(OC)cc(C)c3c2ccn3C(=O)OC(C)(C)C)cc1]

In some embodiments, a compound of formula I is

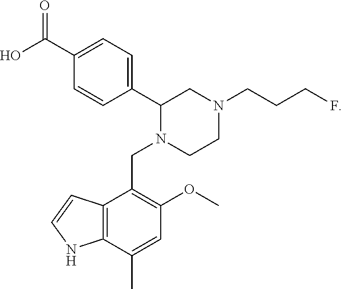 [SMILES: COc1cc(C)c2[nH]ccc2c1CN1CCN(CCCF)CC1c1ccc(C(=O)O)cc1]

In some embodiments, a compound of formula M-I is

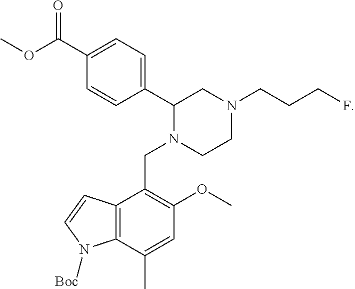 [SMILES: COC(=O)c1ccc(C2CN(CCCF)CCN2Cc2c(OC)cc(C)c3c2ccn3C(=O)OC(C)(C)C)cc1]

In some embodiments, a compound of formula I is

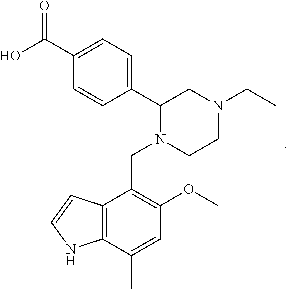 [SMILES: CCN1CCN(Cc2c(OC)cc(C)c3[nH]ccc23)C(c2ccc(C(=O)O)cc2)C1]

In some embodiments, a compound of formula M-I is

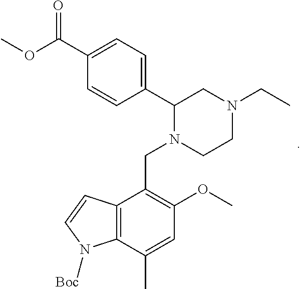 [SMILES: CCN1CCN(Cc2c(OC)cc(C)c3c2ccn3C(=O)OC(C)(C)C)C(c2ccc(C(=O)OC)cc2)C1]

In some embodiments, a compound of formula I is

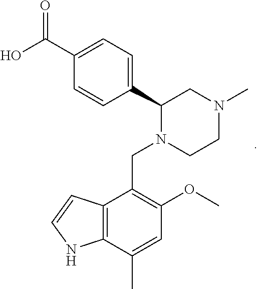 [SMILES: COc1cc(C)c2[nH]ccc2c1CN1CCN(C)C[C@@H]1c1ccc(C(=O)O)cc1]

In some embodiments, a compound of formula M-I is

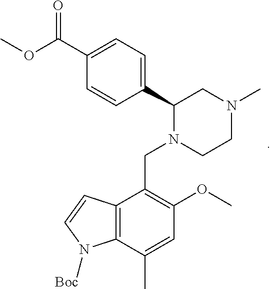

In some embodiments, a compound of formula I is

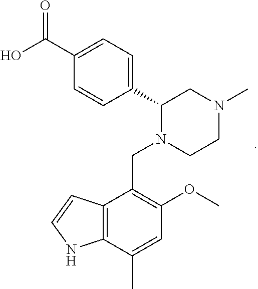 [SMILES: COc1cc(C)c2[nH]ccc2c1CN1CCN(C)C[C@H]1c1ccc(C(=O)O)cc1]

In some embodiments, a compound of formula M-I is

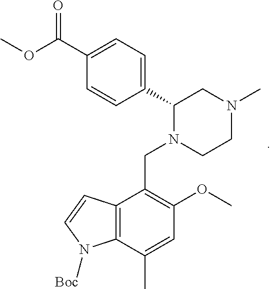

In some embodiments, a compound of formula I is

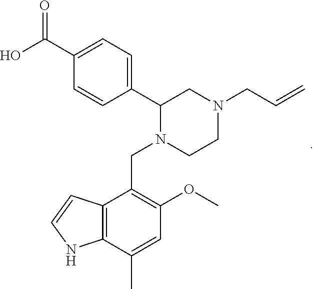 [SMILES: C=CCN1CCN(Cc2c(OC)cc(C)c3[nH]ccc23)C(c2ccc(C(=O)O)cc2)C1]

In some embodiments, a compound of formula M-I is

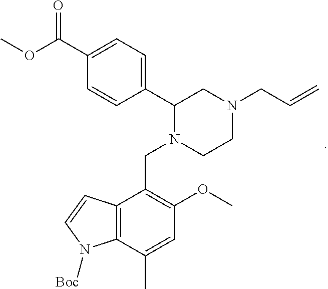 [SMILES: C=CCN1CCN(Cc2c(OC)cc(C)c3c2ccn3C(=O)OC(C)(C)C)C(c2ccc(C(=O)OC)cc2)C1]

In some embodiments, a compound of formula I is

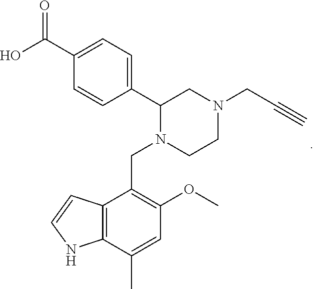 [SMILES: C#CCN1CCN(Cc2c(OC)cc(C)c3[nH]ccc23)C(c2ccc(C(=O)O)cc2)C1]

In some embodiments, a compound of formula M-I is

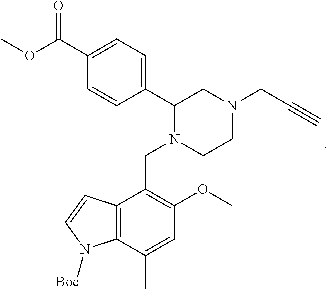 [SMILES: C#CCN1CCN(Cc2c(OC)cc(C)c3c2ccn3C(=O)OC(C)(C)C)C(c2ccc(C(=O)OC)cc2)C1]

In some embodiments, a compound of formula I is

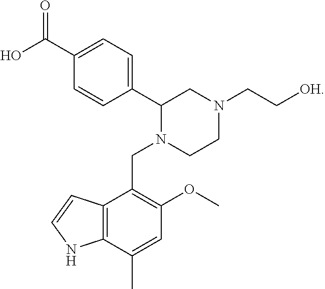 [SMILES: COc1cc(C)c2[nH]ccc2c1CN1CCN(CCO)CC1c1ccc(C(=O)O)cc1]

In some embodiments, a compound of formula M-I is

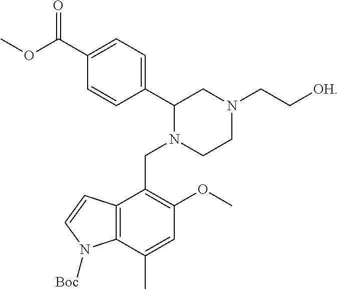 [SMILES: COC(=O)c1ccc(C2CN(CCO)CCN2Cc2c(OC)cc(C)c3c2ccn3C(=O)OC(C)(C)C)cc1]

In some embodiments, a compound of formula I is

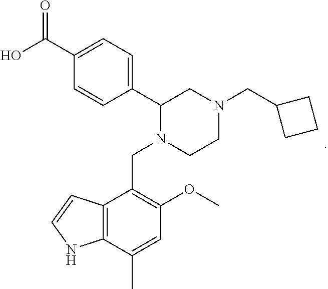 [SMILES: COc1cc(C)c2[nH]ccc2c1CN1CCN(CC2CCC2)CC1c1ccc(C(=O)O)cc1]

In some embodiments, a compound of formula M-I is

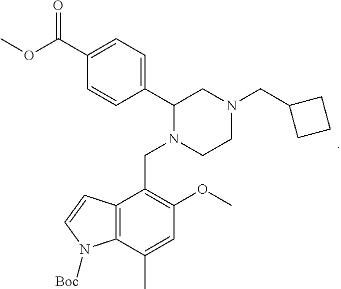 [SMILES: COC(=O)c1ccc(C2CN(CC3CCC3)CCN2Cc2c(OC)cc(C)c3c2ccn3C(=O)OC(C)(C)C)cc1]

In some embodiments, a compound of formula I is

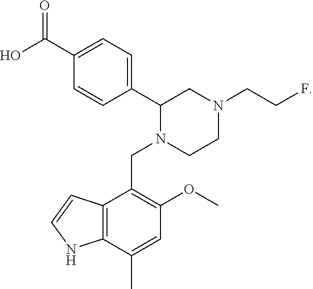 [SMILES: COc1cc(C)c2[nH]ccc2c1CN1CCN(CCF)CC1c1ccc(C(=O)O)cc1]

In some embodiments, a compound of formula M-I is

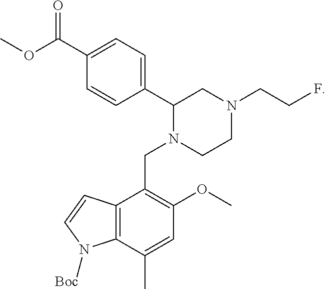 [SMILES: COC(=O)c1ccc(C2CN(CCF)CCN2Cc2c(OC)cc(C)c3c2ccn3C(=O)OC(C)(C)C)cc1]

In some embodiments, a compound of formula I is

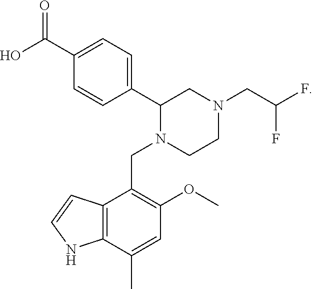 [SMILES: COc1cc(C)c2[nH]ccc2c1CN1CCN(CC(F)F)CC1c1ccc(C(=O)O)cc1]

In some embodiments, a compound of formula M-I is

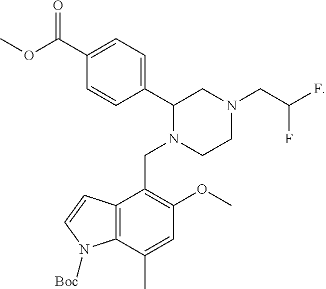 [SMILES: COC(=O)c1ccc(C2CN(CC(F)F)CCN2Cc2c(OC)cc(C)c3c2ccn3C(=O)OC(C)(C)C)cc1]

In some embodiments, a compound of formula I is

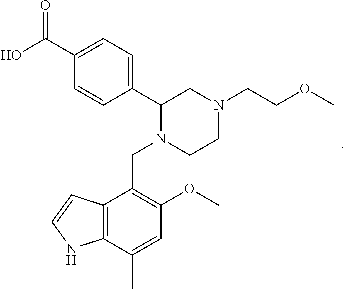 [SMILES: COCCN1CCN(Cc2c(OC)cc(C)c3[nH]ccc23)C(c2ccc(C(=O)O)cc2)C1]

In some embodiments, a compound of formula M-I is

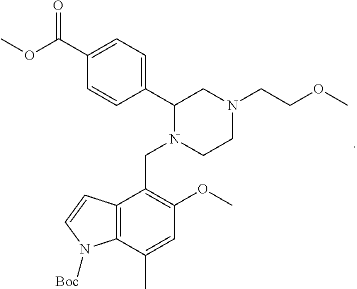

In some embodiments, a compound of formula I is

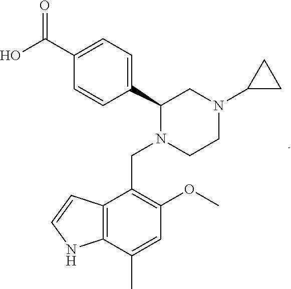 [SMILES: COc1cc(C)c2[nH]ccc2c1CN1CCN(C2CC2)C[C@@H]1c1ccc(C(=O)O)cc1]

In some embodiments, a compound of formula M-I is

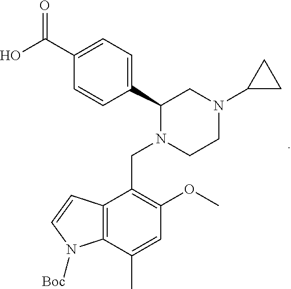 [SMILES: COc1cc(C)c2c(ccn2C(=O)OC(C)(C)C)c1CN1CCN(C2CC2)C[C@@H]1c1ccc(C(=O)O)cc1]

In some embodiments, a compound of formula I is

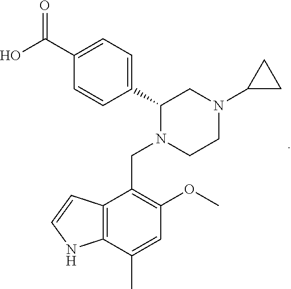 [SMILES: COc1cc(C)c2[nH]ccc2c1CN1CCN(C2CC2)C[C@H]1c1ccc(C(=O)O)cc1]

In some embodiments, a compound of formula M-I is

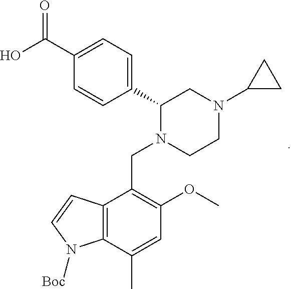 [SMILES: COc1cc(C)c2c(ccn2C(=O)OC(C)(C)C)c1CN1CCN(C2CC2)C[C@H]1c1ccc(C(=O)O)cc1]

In some embodiments, a compound of formula I is

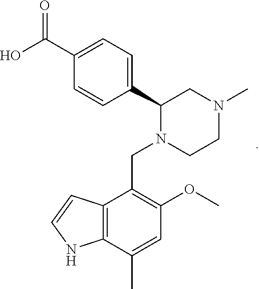 [SMILES: COc1cc(C)c2[nH]ccc2c1CN1CCN(C)C[C@@H]1c1ccc(C(=O)O)cc1]

In some embodiments, a compound of formula M-I is

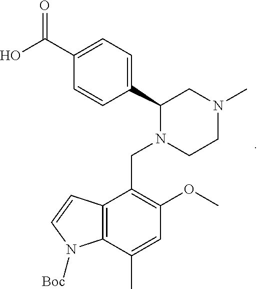 [SMILES: COc1cc(C)c2c(ccn2C(=O)OC(C)(C)C)c1CN1CCN(C)C[C@@H]1c1ccc(C(=O)O)cc1]

In some embodiments, a compound of formula I is

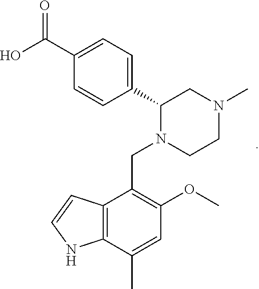 [SMILES: COc1cc(C)c2[nH]ccc2c1CN1CCN(C)C[C@H]1c1ccc(C(=O)O)cc1]

In some embodiments, a compound of formula M-I is

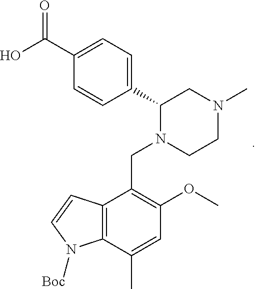 [SMILES: COc1cc(C)c2c(ccn2C(=O)OC(C)(C)C)c1CN1CCN(C)C[C@H]1c1ccc(C(=O)O)cc1]

In some embodiments, a compound of formula I is

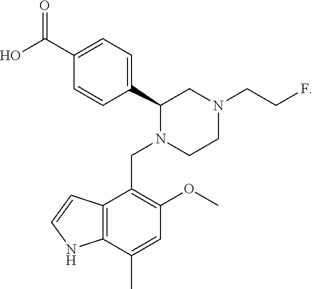 [SMILES: COc1cc(C)c2[nH]ccc2c1CN1CCN(CCF)C[C@@H]1c1ccc(C(=O)O)cc1]

In some embodiments, a compound of formula M-I is

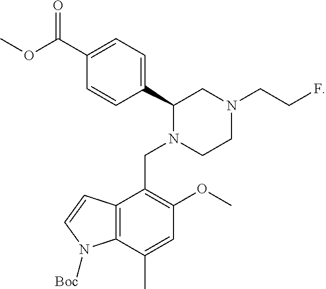 [SMILES: COC(=O)c1ccc([C@H]2CN(CCF)CCN2Cc2c(OC)cc(C)c3c2ccn3C(=O)OC(C)(C)C)cc1]

In some embodiments, a compound of formula I is

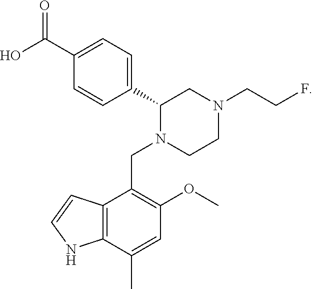 [SMILES: COc1cc(C)c2[nH]ccc2c1CN1CCN(CCF)C[C@H]1c1ccc(C(=O)O)cc1]

In some embodiments, a compound of formula M-I is

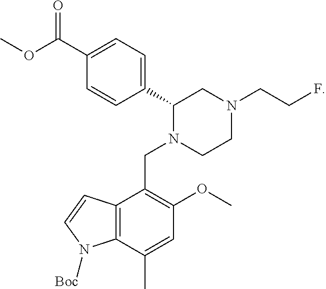 [SMILES: COC(=O)c1ccc([C@@H]2CN(CCF)CCN2Cc2c(OC)cc(C)c3c2ccn3C(=O)OC(C)(C)C)cc1]

In some embodiments, a compound of formula I is

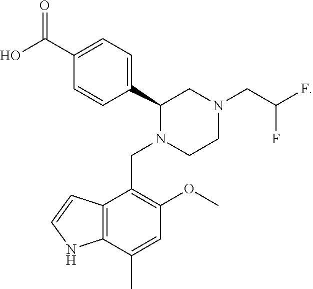 [SMILES: COc1cc(C)c2[nH]ccc2c1CN1CCN(CC(F)F)C[C@@H]1c1ccc(C(=O)O)cc1]

In some embodiments, a compound of formula M-I is

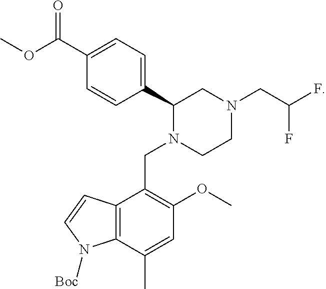 [SMILES: COC(=O)c1ccc([C@H]2CN(CC(F)F)CCN2Cc2c(OC)cc(C)c3c2ccn3C(=O)OC(C)(C)C)cc1]

In some embodiments, a compound of formula I is

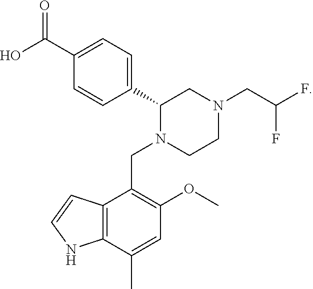 [SMILES: COc1cc(C)c2[nH]ccc2c1CN1CCN(CC(F)F)C[C@H]1c1ccc(C(=O)O)cc1]

In some embodiments, a compound of formula M-I is

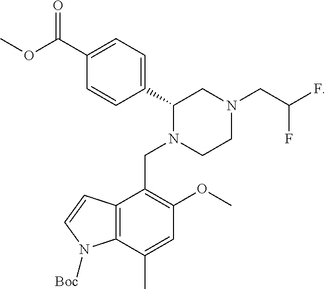 [SMILES: COC(=O)c1ccc([C@@H]2CN(CC(F)F)CCN2Cc2c(OC)cc(C)c3c2ccn3C(=O)OC(C)(C)C)cc1]

In some embodiments, R^{6′} is R^{6′} is hydrogen. In some embodiments, R^{6′} is a primary, secondary or tertiary group. In some embodiments, at least hydrogen is bonded to the carbon in R^{6′} through which R^{6′} is bonded to the rest of a molecule. In some embodiments, R^{6′} is C₁-C₆ aliphatic, R¹⁰, —CH₂—C₃-C₆ cycloaliphatic, —CH₂—R¹⁰, —CH₂-(hydroxy C₁-C₄ alkyl), —CH₂-phenyl, —CH₂-(amino C₁-C₄ alkyl), or —CH₂-(mono- and di-C₁-C₄ alkylamino C₁-C₄ alkyl), each of which is independently optionally substituted as described herein. In some embodiments, R¹⁰ is heterocyclyl optionally substituted as described herein. In some embodiments, a method comprises a step for preparing a compound of formula M-I or a salt thereof, which step comprises a reductive amination reaction comprising contacting an aldehyde or ketone with a compound of formula M-II or a salt thereof in the presence of a reducing agent (e.g., triacetoxyborohydride):

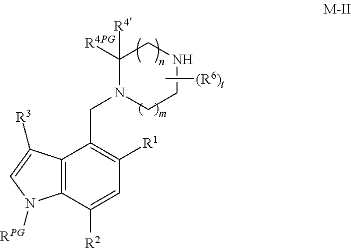

In some embodiments, an aldehyde or ketone is of such a structure that when its —C(O)— group is replaced with —CH₂—, a compound of H—R^{6′} is formed. Various reductive amination technologies are available in the art and can be utilized as described herein.

Various reduction technologies may be utilized in accordance with the present disclosure. In some embodiments, a compound of formula M-II is

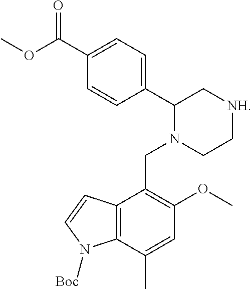

In some embodiments, a compound of formula M-II is

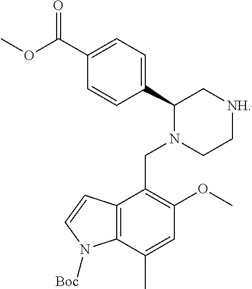

In some embodiments, a compound of formula M-II is

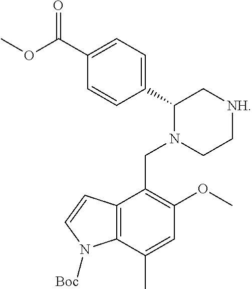

In some embodiments, an aldehyde or ketone is formaldehyde. In some embodiments, a compound of formula M-I is

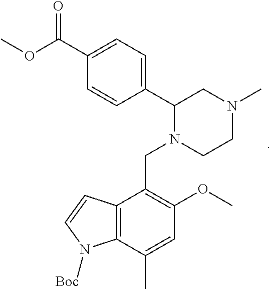

In some embodiments, an aldehyde or ketone is acetone. In some embodiments, a compound of formula M-I is

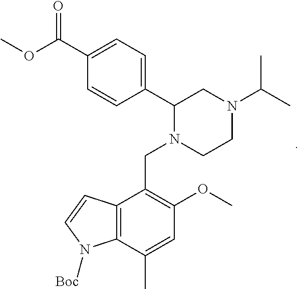 [SMILES: COC(=O)c1ccc(C2CN(C(C)C)CCN2Cc2c(OC)cc(C)c3c2ccn3C(=O)OC(C)(C)C)cc1]

In some embodiments, an aldehyde or ketone is cyclopropanecarboxaldehyde. In some embodiments, a compound of formula M-I is

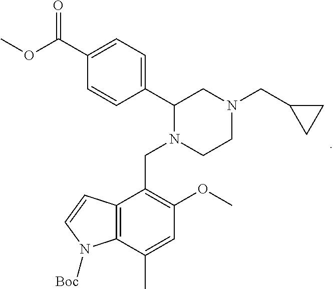 [SMILES: COC(=O)c1ccc(C2CN(CC3CC3)CCN2Cc2c(OC)cc(C)c3c2ccn3C(=O)OC(C)(C)C)cc1]

In some embodiments, an aldehyde or ketone is isobutyraldehyde. In some embodiments, a compound of formula M-I is

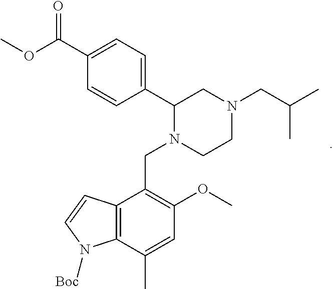 [SMILES: COC(=O)c1ccc(C2CN(CC(C)C)CCN2Cc2c(OC)cc(C)c3c2ccn3C(=O)OC(C)(C)C)cc1]

In some embodiments, an aldehyde or ketone is cyclobutanone. In some embodiments, a compound of formula M-I is

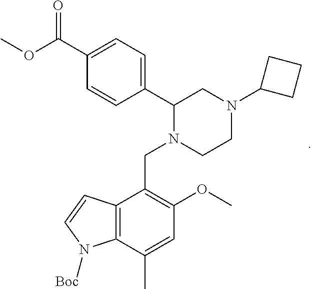 [SMILES: COC(=O)c1ccc(C2CN(C3CCC3)CCN2Cc2c(OC)cc(C)c3c2ccn3C(=O)OC(C)(C)C)cc1]

In some embodiments, an aldehyde or ketone is propionaldehyde. In some embodiments, a compound of formula M-I is

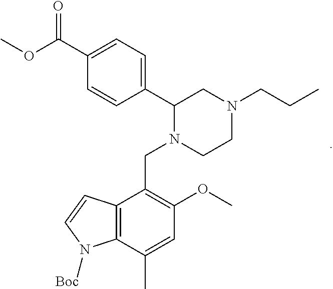 [SMILES: CCCN1CCN(Cc2c(OC)cc(C)c3c2ccn3C(=O)OC(C)(C)C)C(c2ccc(C(=O)OC)cc2)C1]

In some embodiments, an aldehyde or ketone is cyclopentanone. In some embodiments, a compound of formula M-I is

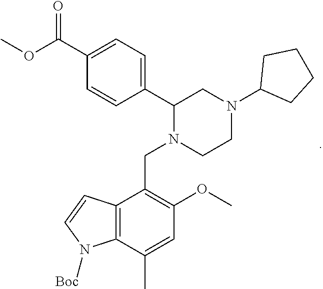 [SMILES: COC(=O)c1ccc(C2CN(C3CCCC3)CCN2Cc2c(OC)cc(C)c3c2ccn3C(=O)OC(C)(C)C)cc1]

In some embodiments, an aldehyde or ketone is 2-butanone. In some embodiments, a compound of formula M-I is

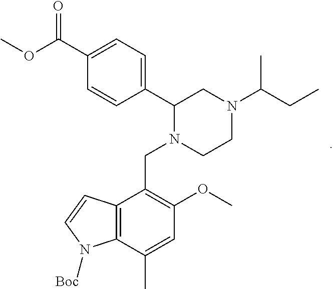 [SMILES: CCC(C)N1CCN(Cc2c(OC)cc(C)c3c2ccn3C(=O)OC(C)(C)C)C(c2ccc(C(=O)OC)cc2)C1]

In some embodiments, an aldehyde or ketone is butyraldehyde. In some embodiments, a compound of formula M-I is

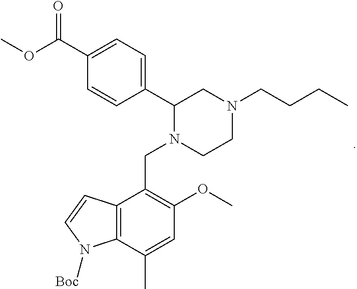 [SMILES: CCCCN1CCN(Cc2c(OC)cc(C)c3c2ccn3C(=O)OC(C)(C)C)C(c2ccc(C(=O)OC)cc2)C1]

In some embodiments, an aldehyde or ketone is 2-methylbutanal. In some embodiments, a compound of formula M-I is

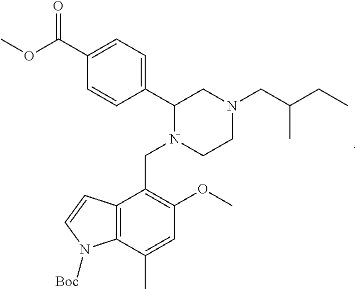 [SMILES: CCC(C)CN1CCN(Cc2c(OC)cc(C)c3c2ccn3C(=O)OC(C)(C)C)C(c2ccc(C(=O)OC)cc2)C1]

In some embodiments, an aldehyde or ketone is 3-methylbutanal. In some embodiments, a compound of formula M-I is

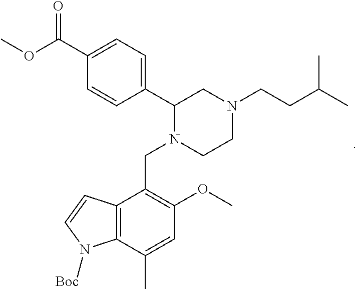 [SMILES: COC(=O)c1ccc(C2CN(CCC(C)C)CCN2Cc2c(OC)cc(C)c3c2ccn3C(=O)OC(C)(C)C)cc1]

In some embodiments, an aldehyde or ketone is cyclopentanecarboxaldehyde. In some embodiments, a compound of formula M-I is

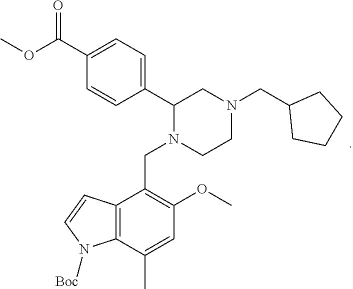 [SMILES: COC(=O)c1ccc(C2CN(CC3CCCC3)CCN2Cc2c(OC)cc(C)c3c2ccn3C(=O)OC(C)(C)C)cc1]

In some embodiments, an aldehyde or ketone is 3-oxetanone. In some embodiments, a compound of formula M-I is

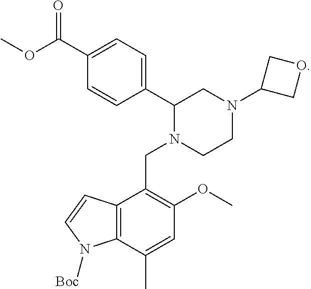 [SMILES: COC(=O)c1ccc(C2CN(C3COC3)CCN2Cc2c(OC)cc(C)c3c2ccn3C(=O)OC(C)(C)C)cc1]

In some embodiments, an aldehyde or ketone is aldehyde. In some embodiments, a compound of formula M-I is

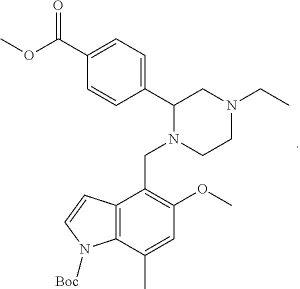 [SMILES: CCN1CCN(Cc2c(OC)cc(C)c3c2ccn3C(=O)OC(C)(C)C)C(c2ccc(C(=O)OC)cc2)C1]

In some embodiments, an aldehyde or ketone is cyclobutanecarbaldehyde. In some embodiments, a compound of formula M-I is

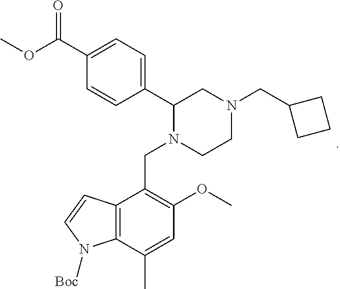 [SMILES: COC(=O)c1ccc(C2CN(CC3CCC3)CCN2Cc2c(OC)cc(C)c3c2ccn3C(=O)OC(C)(C)C)cc1]

In some embodiments, R^{6′} is —C(O)—C₁-C₆ aliphatic optionally substituted as described herein. In some embodiments, a method comprises a step for preparing a compound of formula M-I or a salt thereof, which step comprises an amidation reaction comprising contacting an acylating agent with a compound of formula M-II or a salt thereof. In some embodiments, an acylating agent has the structure of R^{6x1}—C(O)—C₁-C₆ aliphatic or a salt thereof, wherein R^{6x1} is —OH or R^{6x1}—C(O)— is an activated carboxyl group. In some embodiments, R^{6x1} is —Cl. In some embodiments, R^{6x1}—C(O)—C₁-C₆ aliphatic is an anhydride. In some embodiments, a compound of formula M-II is

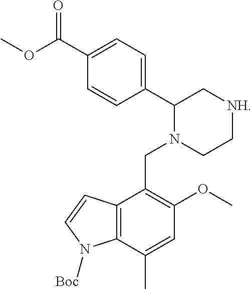

In some embodiments, a compound of formula M-I is

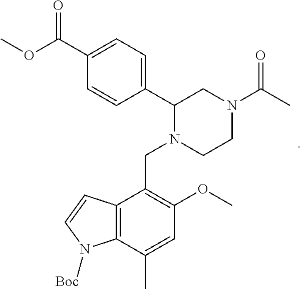 [SMILES: COC(=O)c1ccc(C2CN(C(C)=O)CCN2Cc2c(OC)cc(C)c3c2ccn3C(=O)OC(C)(C)C)cc1]

Various technologies for amidation reactions and acylating agents are available in the art and can be utilized in accordance with the present disclosure.

In some embodiments, R^{6′} is —SO₂—C₁-C₆ aliphatic optionally substituted as described herein. In some embodiments, a method comprises a step for preparing a compound of formula M-I or a salt thereof, which step comprises a sulfonylation reaction comprising contacting a sulfonylation agent with a compound of formula M-II or a salt thereof. In some embodiments, a sulfonylation agent has the structure of R^{6x2}—S(O)₂—C₁-C₆ aliphatic or a salt thereof, wherein R^{6x2}—S(O)₂— is an activated sulfonyl group. In some embodiments, R^{6x2} is —Cl. In some embodiments, a sulfonylation reagent is MsCl. In some embodiments, a compound of formula M-II is

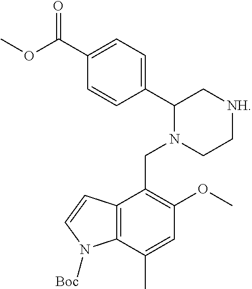

In some embodiments, a compound of formula M-I is

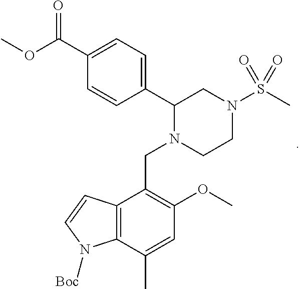

Various technologies for sulfonylation reactions and sulfonylation agents are available in the art and can be utilized in accordance with the present disclosure.

In some embodiments, R^{6′} is installed through an amination reaction. In some embodiments, a method comprises a step for preparing a compound of formula M-I or a salt thereof, which step comprises an amination reaction comprising contacting a sulfonylation agent with a compound of formula M-II or a salt thereof. In some embodiments, a compound of formula M-II or a salt thereof is reacted with a compound having the structure of R^{6x3}—R^{6′}, wherein R^{6x3} is a leaving group, and R^{6′} is as described herein. In some embodiments, R^{6x3} is halogen. In some embodiments, it is —Cl. In some embodiments, it is —Br. In some embodiments, it is —I. In some embodiments, R^{6′} is C₁-C₆ aliphatic as described herein. In some embodiments, R^{6′} is phenyl optionally substituted as described herein. In some embodiments, R^{6′} is phenyl. In some embodiments, R^{6′} is heteroaryl optionally substituted as described herein. In some embodiments, R^{6x3}—R^{6′} is bromobenzene. In some embodiments, a reaction is performed in the presence of a base (e.g., NaOtBu, Et₃N, K₂CO₃, etc.). In some embodiments, a reaction is performed in the presence of a metal reagent, e.g., a catalyst and optionally a ligand (e.g., Pd₂(dba)₃ and RuPhos). In some embodiments, R^{6x3}—R^{6′} is 1-fluoro-3-iodopropane. In some embodiments, R^{6x3}—R^{6′} is allylbromide. In some embodiments, R^{6x3}—R^{6′} is propargyl bromide. In some embodiments, R^{6x3}—R^{6′} is 2-bromoethanol. In some embodiments, R^{6x3}—R^{6′} is 1-fluoro-2-iodoethane. In some embodiments, R^{6x3}—R^{6′} is 2,2-difluoroethyl trifluoromethanesulphonate. In some embodiments, R^{6x3}—R^{6′} is 2-bromoethylmethyl ether.

In some embodiments, a compound of formula M-II is

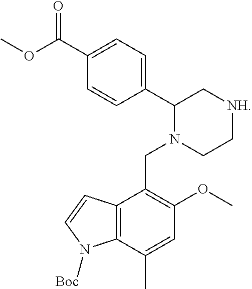

In some embodiments, a compound of formula M-II is

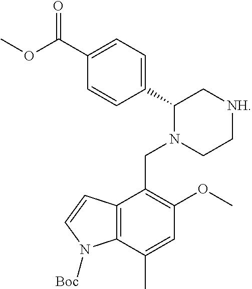

In some embodiments, a compound of formula M-II is

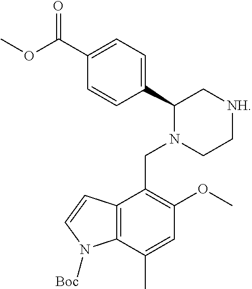

In some embodiments, a compound of formula M-I is

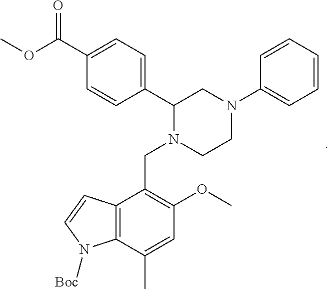 [SMILES: COC(=O)c1ccc(C2CN(c3ccccc3)CCN2Cc2c(OC)cc(C)c3c2ccn3C(=O)OC(C)(C)C)cc1]

In some embodiments, a compound of formula M-I is

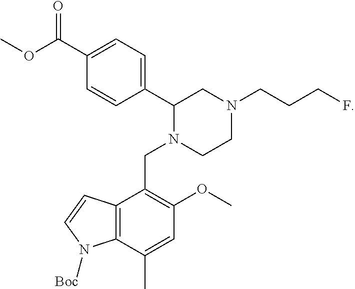 [SMILES: COC(=O)c1ccc(C2CN(CCCF)CCN2Cc2c(OC)cc(C)c3c2ccn3C(=O)OC(C)(C)C)cc1]

In some embodiments, a compound of formula M-I is

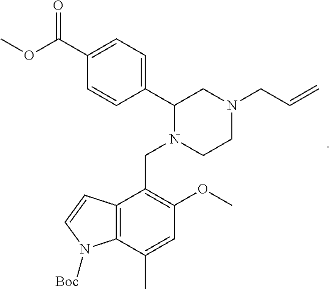 [SMILES: C=CCN1CCN(Cc2c(OC)cc(C)c3c2ccn3C(=O)OC(C)(C)C)C(c2ccc(C(=O)OC)cc2)C1]

In some embodiments, a compound of formula M-I is

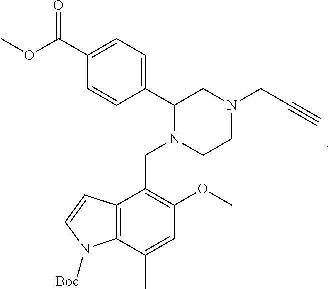 [SMILES: C#CCN1CCN(Cc2c(OC)cc(C)c3c2ccn3C(=O)OC(C)(C)C)C(c2ccc(C(=O)OC)cc2)C1]

In some embodiments, a compound of formula M-I is

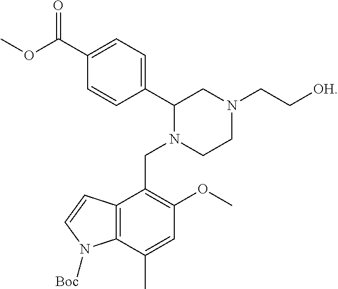 [SMILES: COC(=O)c1ccc(C2CN(CCO)CCN2Cc2c(OC)cc(C)c3c2ccn3C(=O)OC(C)(C)C)cc1]

In some embodiments, a compound of formula M-I is

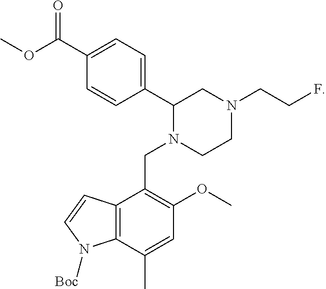 [SMILES: COC(=O)c1ccc(C2CN(CCF)CCN2Cc2c(OC)cc(C)c3c2ccn3C(=O)OC(C)(C)C)cc1]

In some embodiments, a compound of formula M-I is

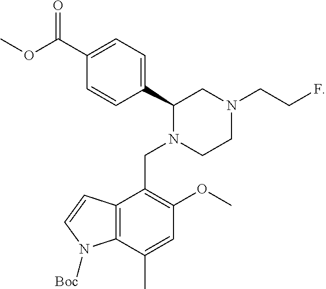 [SMILES: COC(=O)c1ccc([C@H]2CN(CCF)CCN2Cc2c(OC)cc(C)c3c2ccn3C(=O)OC(C)(C)C)cc1]

In some embodiments, a compound of formula M-I is

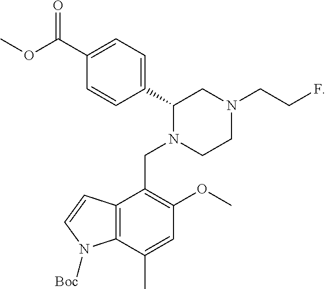 [SMILES: COC(=O)c1ccc([C@@H]2CN(CCF)CCN2Cc2c(OC)cc(C)c3c2ccn3C(=O)OC(C)(C)C)cc1]

In some embodiments, a compound of formula M-I is

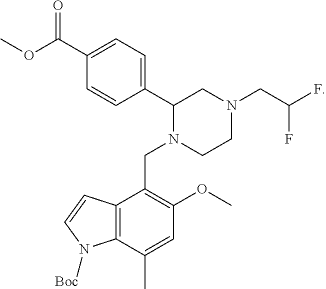 [SMILES: COC(=O)c1ccc(C2CN(CC(F)F)CCN2Cc2c(OC)cc(C)c3c2ccn3C(=O)OC(C)(C)C)cc1]

In some embodiments, a compound of formula M-I is

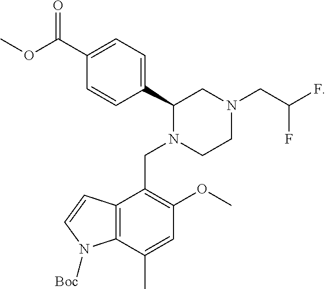 [SMILES: COC(=O)c1ccc([C@H]2CN(CC(F)F)CCN2Cc2c(OC)cc(C)c3c2ccn3C(=O)OC(C)(C)C)cc1]

In some embodiments, a compound of formula M-I is

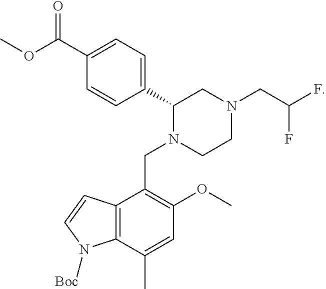 [SMILES: COC(=O)c1ccc([C@@H]2CN(CC(F)F)CCN2Cc2c(OC)cc(C)c3c2ccn3C(=O)OC(C)(C)C)cc1]

In some embodiments, a compound of formula M-I is

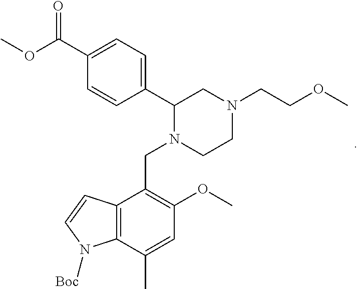

Various technologies for amination reactions are available in the art and can be utilized in accordance with the present disclosure. Certain examples are described in the Examples. In some embodiments, amination is performed in the presence of a catalyst system (e.g., Pd₂(dba)₃ and RuPhos) and/or a base (e.g., NaOtBu).

Various technologies for sulfonylation reactions and sulfonylation agents are available in the art and can be utilized in accordance with the present disclosure.

In some embodiments, as described herein, t is 0. In some embodiments, m is 1. In some embodiments, n is 1. In some embodiments, R^{4′} is —H. In some embodiments, R¹ is —O—C₁-C₆ alkyl, e.g., —OMe. In some embodiments, R¹ is —H. In some embodiments, R² is —C₁-C₆ alkyl, e.g. —CH₃. In some embodiments, R² is —H. In some embodiments, R³ is —H. In some embodiments, R^{PG} is —Boc. In some embodiments, a compound of formula II is

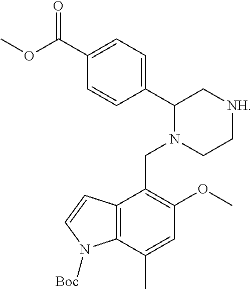

In some embodiments, a compound of formula II is

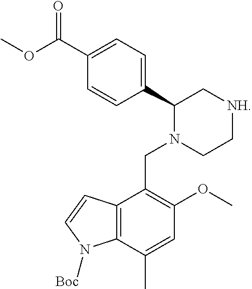

In some embodiments, a compound of formula II is

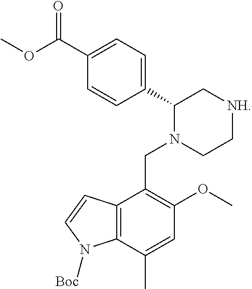

In some embodiments, a method comprises a step for preparing a compound of formula M-II or a salt thereof, which step comprises de-protecting a compound having the structure of M-III or a salt thereof:

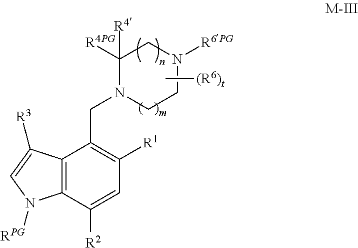

wherein R^{6′PG} is R^{6′} or an amino protecting group. In some embodiments, R^{6′PG} is an amino protecting group. In some embodiments, it can be selectively de-protected in the presence of R^{4PG} and R^{PG}. For example, as utilized in the Examples, in some embodiments, R^{6′PG} is —Bn. In some embodiments, R^{PG} is —Boc. In some embodiments, a compound of formula II is

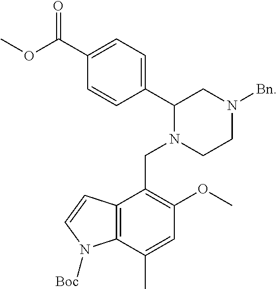 [SMILES: COC(=O)c1ccc(C2CN(Cc3ccccc3)CCN2Cc2c(OC)cc(C)c3c2ccn3C(=O)OC(C)(C)C)cc1]

In some embodiments, a method comprises reacting a compound of formula M-IV or a salt thereof:

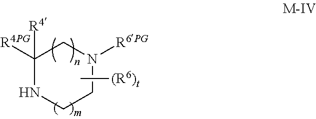

wherein each variable is independently as described herein,
with a compound of formula M-V or a salt thereof:

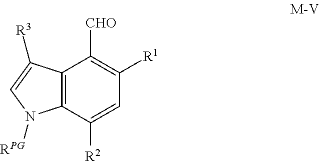

wherein each variable is independently as described herein,
under a condition to provide a compound of formula M-III or a salt thereof. In some embodiments, a condition is a reductive amination condition. Various reductive amination conditions can be utilized in accordance with the present disclosure.

In some embodiments, the present disclosure provides a method comprising protecting a group and/or de-protecting a group of a compound of formula M-VI or a salt thereof:

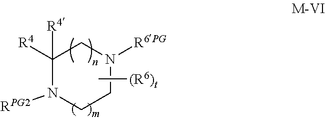

wherein R^{PG2} is an amino protecting group, and each other variable is independently as described herein, to provide a compound of formula M-IV or a salt thereof.

In some embodiments, R⁴ is

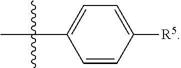

In some embodiments, R⁵ is —COOH. In some embodiments, R^{4PG} is

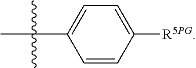

In some embodiments, R^{5PG} is —(O)—R^{8PG}. In some embodiments, R^{5PG} is —COOMe. In some embodiments, R^{PG2} and R^{6′PG} can be selectively removed in the presence of the other. In some embodiments, R^{PG2} is Boc. In some embodiments, R^{6′PG} is Bn. In some embodiments, a compound of formula M-IV is

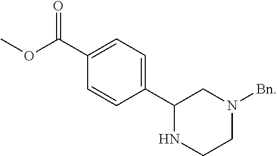 [SMILES: COC(=O)c1ccc(C2CN(Cc3ccccc3)CCN2)cc1]

In some embodiments, a salt of a compound of formula M-IV is

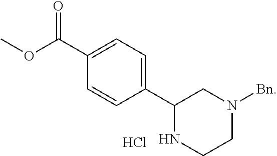 [SMILES: COC(=O)c1ccc(C2CN(Cc3ccccc3)CCN2)cc1 ; Cl]

In some embodiments, as described herein, R^{PG} is Boc. In some embodiments, R¹ is —O—C₁-C₆ alkyl. In some embodiments, R¹ is —OCH₃. In some embodiments, R² is —CH₃. In some embodiments, R³ is —H. In some embodiments, a compound of formula M-V is

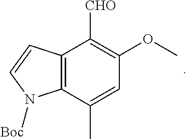 [SMILES: COc1cc(C)c2c(ccn2C(=O)OC(C)(C)C)c1C=O]

In some embodiments, a compound of formula M-VI is

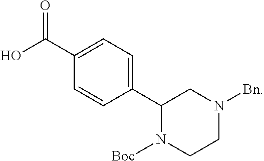 [SMILES: CC(C)(C)OC(=O)N1CCN(Cc2ccccc2)CC1c1ccc(C(=O)O)cc1]

In some embodiments, the present disclosure provides a method comprising converting a compound of formula M-VII or a salt thereof:

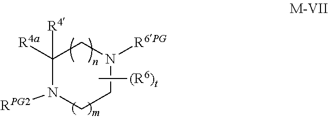

wherein:
R^{4a} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5a} and further substituted with 0 or 1 substituent selected from halogen, C₁-C₄ alkyl, C₁-C₄ alkoxy, hydroxy C₁-C₄ alkyl, hydroxy, and cyanomethyl;
R^{5a} is —CHO, —CH₂CHO, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom; and
   each other variable is independently as described herein, to provide a compound of formula M-VI or a salt thereof.

In some embodiments, R^{4a} is substituted with —CHO. In some embodiments, R⁴ is substituted with —COOH. In some embodiments, R^{4a} is

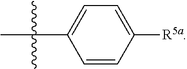

In some embodiments, R^{5a} is —CHO. In some embodiments, R⁴ is

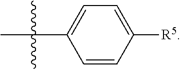

In some embodiments, R⁵ is —COOH. In some embodiments, a reaction is an oxidation reaction which converts an aldehyde into a carboxylic acid group. In some embodiments, t is 0. In some embodiments, R^{PG2} is Boc. In some embodiments, R^{6′PG} is Bn. In some embodiments, a compound of formula M-VII is

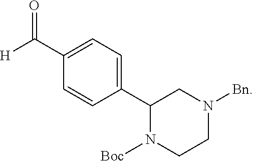 [SMILES: CC(C)(C)OC(=O)N1CCN(Cc2ccccc2)CC1c1ccc(C=O)cc1]

Various such oxidation technologies are available and can be utilized in accordance with the present disclosure.

In some embodiments, a method comprising protecting an amino group, e.g., in a compound of formula M-VIII or a salt thereof:

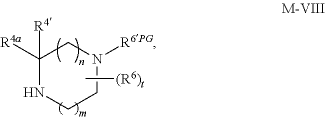

wherein each variable is independently as described herein, to provide a compound of formula M-VII or a salt thereof. In some embodiments, R^{RPG2} is Boc. In some embodiments, R^{4a} is substituted with —CHO. In some embodiments, R^{4a} is

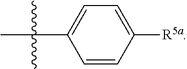

In some embodiments, R^{5a} is —CHO. In some embodiments, t is 0. In some embodiments, a compound of formula M-VIII is

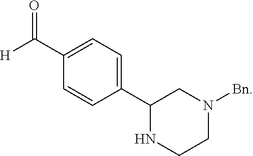 [SMILES: O=Cc1ccc(C2CN(Cc3ccccc3)CCN2)cc1]

In some embodiments, the present disclosure provides a method comprising converting a compound of formula M-IX or a salt thereof:

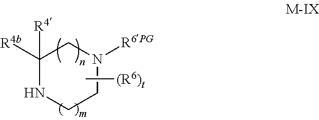

wherein:
R^{4b} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5b} and further substituted with 0 or 1 substituent selected from halogen, C₁-C₄ alkyl, C₁-C₄ alkoxy, hydroxy C₁-C₄ alkyl, hydroxy, and cyanomethyl;
R^{5b} is —CH₂OH, —CH₂ CH₂OH, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom; and
   each other variable is independently as described herein, to provide a compound of formula M-VIII or a salt thereof.

In some embodiments, R^{4b} is substituted with —CH₂OH. In some embodiments, R^{4a} is substituted with —CHO. In some embodiments, R^{4a} is

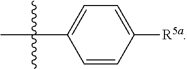

In some embodiments, R^{5a} is —CHO. In some embodiments, R^{4b} is

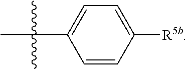

In some embodiments, R^{5b} is —CH₂OH. In some embodiments, a reaction is an oxidation reaction which converts a primary alcohol into an aldehyde. In some embodiments, t is 0. In some embodiments, a compound of formula IX is

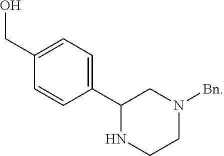 [SMILES: OCc1ccc(C2CN(Cc3ccccc3)CCN2)cc1]

Various oxidation technologies are available and can be utilized in accordance with the present disclosure.

In some embodiments, the present disclosure provides a method comprising reducing a compound of formula M-X or a salt thereof:

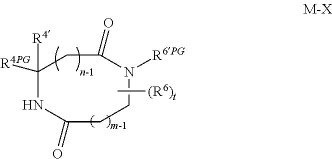

wherein each variable is independently as described herein, to provide a compound of formula M-IX or a salt thereof. In some embodiments, R^{4PG} is

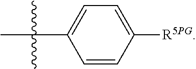

In some embodiments, R^{5PG} is —(O)—R^{8PG}. In some embodiments, R^{5PG} is —COOMe. In some embodiments, n is 1. In some embodiments, m is 1. In some embodiments, R^{6′PG} is —Bn. In some embodiments, a carboxyl or a derivative thereof (e.g., an ester) is reduced to a hydroxyl. In some embodiments, t is 0. In some embodiments, a compound of formula M-X is

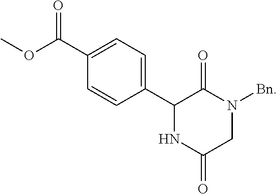 [SMILES: COC(=O)c1ccc(C2NC(=O)CN(Cc3ccccc3)C2=O)cc1]

Various reduction technologies are available and can be utilized in accordance with the present disclosure.

In some embodiments, the present disclosure provides a method comprising converting a compound of formula M-XI or a salt thereof:

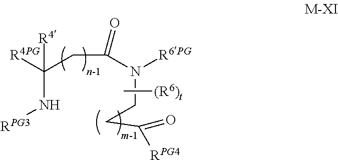

wherein:
R^{PG3} is —H or an amino protecting group;
R^{PG4} is —OH or —C(O)R^{PG4} is a protected carboxyl group; and
each variable is independently as described herein, to provide a compound of formula M-X or a salt thereof.

In some embodiments, R^{PG3} is hydrogen. In some embodiments, it is an amino protecting group. In some embodiments, it is —Boc. In some embodiments, R^{PG4} is —OH. In some embodiments, it is a carboxy protecting group. In some embodiments, it is —O-optionally substituted C₁-C₆ aliphatic. In some embodiments, it is —O-optionally substituted C₁-C₆ alkyl. In some embodiments, it is —OMe. In some embodiments, t is 0. In some embodiments, a compound of formula M-XI is

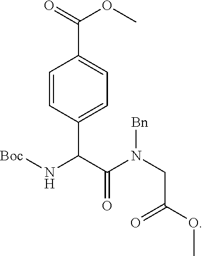 [SMILES: COC(=O)CN(Cc1ccccc1)C(=O)C(NC(=O)OC(C)(C)C)c1ccc(C(=O)OC)cc1]

In some embodiments, the present disclosure provides a method comprising reacting a compound of formula M-XII or a salt thereof:

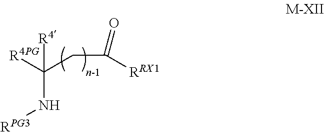

wherein R^{RX1} is —OH or —C(O)R^{RX1} is an activated carboxyl group, and each other variable is independently as described herein, with a compound of formula M-XIII or a salt thereof:

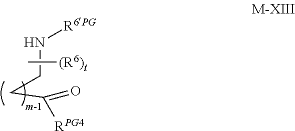

wherein each variable is independently as described herein, to provide a compound of formula M-XI or a salt thereof.

In some embodiments, R^{PG3} is Boc. In some embodiments, R^{RX1} is —OH. In some embodiments, a compound of formula M-XII is

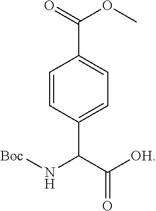 [SMILES: COC(=O)c1ccc(C(NC(=O)OC(C)(C)C)C(=O)O)cc1]

In some embodiments, R^{6′PG} is —Bn. In some embodiments, t is 0. In some embodiments, R^{PG4} is optionally substituted —O—C₁-C₆ aliphatic. In some embodiments, it optionally substituted —O—C₁-C₆ alkyl. In some embodiments, it is —OCH₃. In some embodiments, a compound of formula M-XIII is

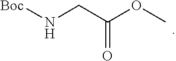

In some embodiments, the present disclosure provides a method, comprising converting a compound of formula M-XIV or a salt thereof:

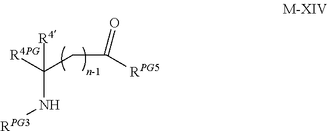

wherein:
—C(O)R^{PG5} is protected carboxyl, and each other variable is independently as described herein, to a compound of formula M-XII or a salt thereof.

In some embodiments, —C(O)R^{PG5} is —C(O)—O-optionally substituted C₁-C₆ aliphatic. In some embodiments, it is —O-optionally substituted C₁-C₆ alkyl. In some embodiments, it is —OMe. In some embodiments, —C(O)R^{PG5} can be selectively deprotected in the presence of R^{PG3}. In some embodiments, R^{PG3} is Boc. In some embodiments, n is 0. In some embodiments, a compound of formula M-XIV is

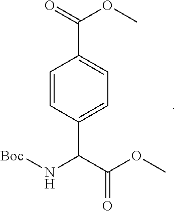 [SMILES: COC(=O)c1ccc(C(NC(=O)OC(C)(C)C)C(=O)OC)cc1]

In some embodiments, a method comprises reacting a compound of formula M-IV′ or a salt thereof:

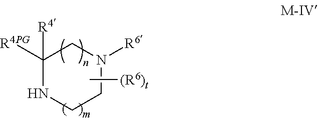

wherein each variable is independently as described herein,
with a compound of formula M-V or a salt thereof:

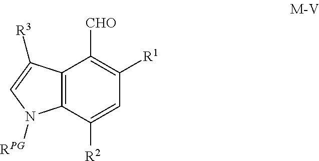

wherein each variable is independently as described herein,
under a condition to provide a compound of formula M-I or a salt thereof. In some embodiments, a condition is a reductive amination condition. Various reductive amination conditions can be utilized in accordance with the present disclosure.

In some embodiments, R^{6′} is cyclopropyl. In some embodiments, t is 0. In some embodiments, m is 1. In some embodiments, n is 1. In some embodiments, R^{4′} is hydrogen. In some embodiments, a compound of formula M-IV′ is

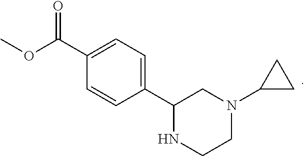 [SMILES: COC(=O)c1ccc(C2CN(C3CC3)CCN2)cc1]

In some embodiments, a salt of a compound of formula M-IV′ is

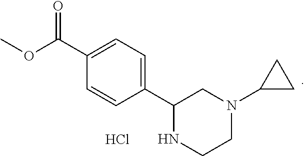 [SMILES: COC(=O)c1ccc(C2CN(C3CC3)CCN2)cc1 ; Cl]

In some embodiments, a compound of formula M-V is

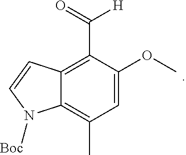 [SMILES: COc1cc(C)c2c(ccn2C(=O)OC(C)(C)C)c1C=O]

In some embodiments, a compound of formula M-I is

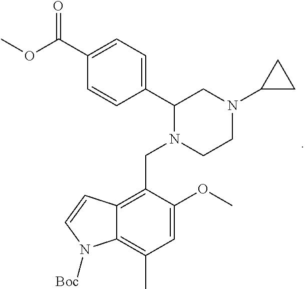 [SMILES: COC(=O)c1ccc(C2CN(C3CC3)CCN2Cc2c(OC)cc(C)c3c2ccn3C(=O)OC(C)(C)C)cc1]

In some embodiments, the present disclosure provides a method comprising de-protecting a group of a compound of formula M-XV or a salt thereof:

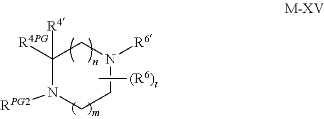

wherein each variable is independently as described herein, to provide a compound of formula M-IV′ or a salt thereof. In some embodiments, R^{PG2} is Boc. In some embodiments, a compound of formula M-XV is

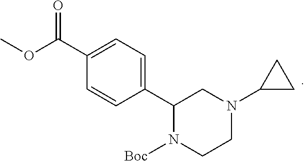 [SMILES: COC(=O)c1ccc(C2CN(C3CC3)CCN2C(=O)OC(C)(C)C)cc1]

In some embodiments, a method comprises a step for preparing a compound of formula M-XV or a salt thereof, which step comprises a reductive amination reaction comprising contacting an aldehyde or ketone with a compound of formula M-XVI or a salt thereof in the presence of a reducing agent (e.g., triacetoxyborohydride):

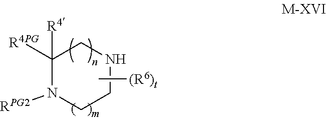

In some embodiments, a method comprises a step for preparing a compound of formula M-XV or a salt thereof, which step comprises amination reaction comprising contacting a suitable reagent with a compound of formula M-XVI or a salt thereof to provide a compound of formula M-XV or a salt thereof. In some embodiments, a method comprises a step for preparing a compound of formula M-XV or a salt thereof, which step comprises amidation or sulfonylation reaction comprising contacting a suitable reagent with a compound of formula M-XVI or a salt thereof to provide a compound of formula M-XV or a salt thereof. In some embodiments, a sulfonylation agent has the structure of R^{6x2}—S(O)₂—C₁-C₆ aliphatic or a salt thereof as described herein. In some embodiments, an acylating agent has the structure of R^{6x1}—C(O)—C₁-C₆ aliphatic or a salt thereof as described herein.

Various reduction technologies may be utilized in accordance with the present disclosure. In some embodiments, a compound of formula M-XVI is

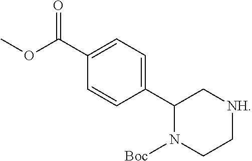 [SMILES: COC(=O)c1ccc(C2CNCCN2C(=O)OC(C)(C)C)cc1]

In some embodiments, a suitable reagent is a boronic acid reagent. In some embodiments, a suitable reagent is cyclopropylboronic acid. In some embodiments, a reaction is performed with a base (e.g. sodium carbonate) and/or a metal salt (e.g., copper (II) acetate).

In some embodiments, the present disclosure provides a method comprising de-protecting a group of a compound of formula M-XVII or a salt thereof:

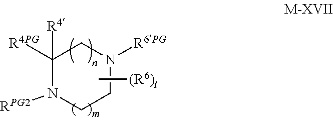

wherein each variable is independently as described herein, to provide a compound of formula M-XVI or a salt thereof. In some embodiments, R^{PG2} is Boc. In some embodiments, R^{6′PG} is —Bn. In some embodiments, a compound of formula M-XVII is

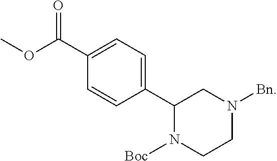 [SMILES: COC(=O)c1ccc(C2CN(Cc3ccccc3)CCN2C(=O)OC(C)(C)C)cc1]

In some embodiments, the present disclosure provides a method comprising protecting a group of a compound of formula M-IV or a salt thereof to provide a compound of formula M-XVII or a salt thereof. In some embodiments, R^{PG2} is Boc. In some embodiments, R^{6′PG} is —Bn. In some embodiments, a compound of formula M-IV is

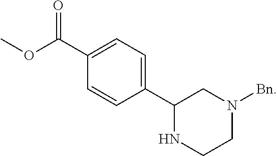 [SMILES: COC(=O)c1ccc(C2CN(Cc3ccccc3)CCN2)cc1]

In some embodiments, a salt of a compound of formula M-IV is

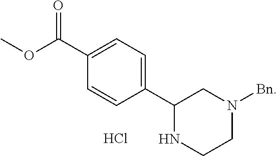 [SMILES: COC(=O)c1ccc(C2CN(Cc3ccccc3)CCN2)cc1 ; Cl]

In various embodiments, reactions are performed in solvent systems, e.g., those exemplified in the samples. In some embodiments, a solvent system is or comprises a polar solvent. In some embodiments, a solvent system is or comprises a non-polar solvent. In some embodiments, a solvent system is or comprises a hydrocarbon solvent. In some embodiments, a solvent system is or comprises a protic solvent. In some embodiments, a solvent system is or comprises water. In some embodiments, a solvent system is anhydrous. In some embodiments, a solvent system is or comprises an alcohol.

In some embodiments, certain reactions may be performed at room temperature. In some embodiments, certain reactions are performed at about 23° C. In some embodiments, certain reactions are performed at lowered temperatures, e.g., 0° C., −10° C., −20° C., etc. In some embodiments, certain reactions are performed at increased temperatures, e.g., about 30-200, 40-150, or about 30, 40, 50, 60, 70, 80, 90, 100, 110, or 150° C. In some embodiments, temperatures may change during reactions, e.g., increasing from a lowered temperature, decreasing from an increased temperature, or combinations thereof. In some embodiments, certain reactions may be performed at increased or decreased pressure.

Many technologies are available for isolating, purifying or characterizing chemical compounds and can be utilized in accordance with the present disclosure. For example, in some embodiments, one or more of extraction, filtration, crystallization, and distillation are utilized to isolate desired products and/or remove undesired byproducts or impurities. In some embodiments, chromatography, e.g., column chromatography, GC, HPLC, SFC, etc. is utilized for separation and/or characterization.

In some embodiments, compounds comprise chiral elements, e.g., carbon chiral centers and two or more stereoisomers (e.g., enantiomers, diastereomers, etc.) may be formed. In some embodiments, stereoselective technologies are utilized so desired stereoisomers are formed. In some embodiments, separation technologies, e.g., crystallization in an asymmetric environment (e.g., with a chirally pure reagent), chiral chromatography, etc., are utilized to isolated, purify or characterize stereoisomers. In some embodiments, a stereoisomer is an enantiomer. In some embodiments, a provided compound is enriched for a particular stereoisomer. In some embodiments, a provided compound is enriched for a particular diastereomer. In some embodiments, a provided compound is enriched for a particular enantiomer. In some embodiments, an enrichment level is a stereopurity, e.g., a diastereomeric purity, an enantiomeric purity, etc. as described herein. In some embodiments, an enrichment level is about 80%-100% (e.g., about 80%, 85%, 90%, 91%, 92%, 93%, 94%, 95%, 96%, 97%, 98%, 99%, 99.5%, 99.9% or more, or about 85%-100%, 90%-100%, 91%-100%, 92%-100%, 93%-100%, 94%-100%, 95%-100%, 96%-100%, 97%-100%, 98%-100%, 99%-100%, 95-99%, 95%-99.5%, 95%-99.9%, etc) as described herein.

Those skilled in the art appreciate that in addition to the compounds specified in provided methods, other reagents and/or conditions may be utilized in accordance with the present disclosure. For example, in some de-protecting reactions, an acid or base may be utilized. In some embodiments, in oxidation reactions, oxidation reagents are utilized. In some embodiments, in reduction reactions, reduction reagents are utilized. Many suitable reagents and conditions are reported and can be utilized in accordance with the present disclosure.

Properties and activities of provided compounds and compositions may be assessed using a variety of technologies in accordance with the present disclosure. In some embodiments, modulation of C3 convertase activity is assessed. In some embodiments, inhibition of C3 convertase is assessed. In some embodiments, modulation of complement activation is assessed. In some embodiments, inhibition of complement activation is assessed. Certain useful technologies are described in the Examples.

Among other things, the present disclosure provides the following Example Embodiments:

1. A compound having the structure of formula I:

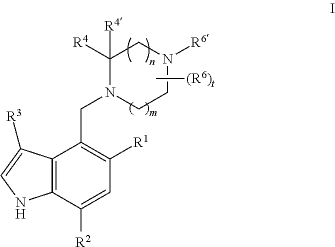

or a pharmaceutically acceptable salt thereof, wherein:
m is 0, 1, or 2;
n is 1, or 2;
R¹ is hydrogen, halogen, hydroxy, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷, phenyl or 5- or 6-membered heteroaryl having 1, 2 or 3 ring heteroatoms independently selected from nitrogen, oxygen and sulfur, wherein the phenyl or heteroaryl is optionally substituted with 0, 1, or 2 C₁-C₄ alkyl groups, and wherein alkyl and haloalkyl are optionally substituted with 0 or 1 hydroxy;
R⁴ is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl;
R^{4′} is hydrogen, C₁-C₄ alkyl, or hydroxy C₁-C₄ alkyl;
R⁵ is —C(O)R⁸, —CH₂C(O)R¹, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
R⁷ is hydroxy, C₁-C₄ alkoxy, amino, or mono- or di-C₁-C₄ alkylamino;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   2. A compound having the structure of formula M-I:

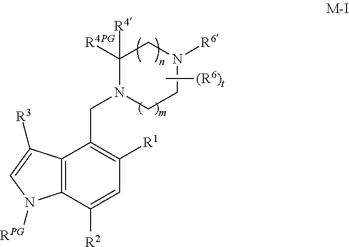

or a salt thereof, wherein:
R^{PG} is an amino protecting group,
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 0, 1, or 2;
n is 1, or 2;
R¹ is hydrogen, halogen, hydroxy, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷, phenyl or 5- or 6-membered heteroaryl having 1, 2 or 3 ring heteroatoms independently selected from nitrogen, oxygen and sulfur, wherein the phenyl or heteroaryl is optionally substituted with 0, 1, or 2 C₁-C₄ alkyl groups, and wherein alkyl and haloalkyl are optionally substituted with 0 or 1 hydroxy;
R^{4′} is hydrogen, C₁-C₄ alkyl, or hydroxy C₁-C₄ alkyl;
R⁵ is —C(O)R⁸, —CH₂C(O)R¹, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
R⁷ is hydroxy, C₁-C₄ alkoxy, amino, or mono- or di-C₁-C₄ alkylamino;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   3. A compound having the structure of formula M-II:

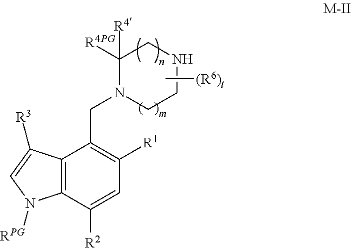

or a salt thereof, wherein:
R^{PG} is an amino protecting group,
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 0, 1, or 2;
n is 1, or 2;
R¹ is hydrogen, halogen, hydroxy, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷, phenyl or 5- or 6-membered heteroaryl having 1, 2 or 3 ring heteroatoms independently selected from nitrogen, oxygen and sulfur, wherein the phenyl or heteroaryl is optionally substituted with 0, 1, or 2 C₁-C₄ alkyl groups, and wherein alkyl and haloalkyl are optionally substituted with 0 or 1 hydroxy;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R⁷ is hydroxy, C₁-C₄ alkoxy, amino, or mono- or di-C₁-C₄ alkylamino;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C1-3 acyl, cyano C₁-C₄ alkyl, C₃-C₆ cycloalkyl, or 3-6 membered heterocyclyl having 1-4 ring atoms independently selected from nitrogen, oxygen and sulfur.
   4. A compound having the structure of formula M-III:

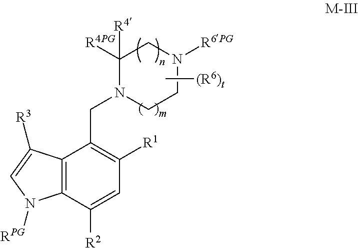

or a salt thereof, wherein:
R^{PG} is an amino protecting group,
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 0, 1, or 2;
n is 1, or 2;
R¹ is hydrogen, halogen, hydroxy, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷, phenyl or 5- or 6-membered heteroaryl having 1, 2 or 3 ring heteroatoms independently selected from nitrogen, oxygen and sulfur, wherein the phenyl or heteroaryl is optionally substituted with 0, 1, or 2 C₁-C₄ alkyl groups, and wherein alkyl and haloalkyl are optionally substituted with 0 or 1 hydroxy;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
R⁷ is hydroxy, C₁-C₄ alkoxy, amino, or mono- or di-C₁-C₄ alkylamino;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   5. The compound of any one of the preceding Embodiments, wherein R¹ is C₁-C₆ alkoxy, C₁-C₆ alkoxy C₁-C₆ alkoxy, C₃-C₆ cycloalkyl C₁-C₆ alkoxy, or halo C₁-C₆ alkoxy.
   6. The compound of any one of the preceding Embodiments, wherein R¹ is C₁-C₆ alkoxy.
   7. The compound of any one of the preceding Embodiments, wherein R¹ is C₁-C₄ alkoxy.
   8. The compound of any one of the preceding Embodiments, wherein R¹ is C₁-C₂ alkoxy.
   9. The compound of any one of the preceding Embodiments, wherein R¹ is methoxy.
   10. The compound of any one of Embodiments 1-4, wherein R¹ is C₁-C₆ alkyl.
   11. The compound of Embodiment 10, wherein R¹ is C₁-C₄ alkyl.
   12. The compound of Embodiment 10, wherein R¹ is C₁-C₂ alkyl.
   13. The compound of any one of Embodiments 1-4, wherein R¹ is C₃-C₅ cycloalkyl.
   14. The compound of Embodiment 13, wherein R¹ is cyclopropyl.
   15. The compound of any one of the preceding Embodiments, wherein R² is C₁-C₆ alkyl.
   16. The compound of any one of the preceding Embodiments, wherein R² is C₁-C₄ alkyl.
   17. The compound of any one of the preceding Embodiments, wherein R² is C₁-C₂ alkyl.
   18. The compound of any one of the preceding Embodiments, wherein R² is methyl.
   19. The compound of any one of the preceding Embodiments, wherein R³ is hydrogen.
   20. The compound of any one of Embodiments 1-17, wherein R³ is halogen.
   21. The compound of any one of Embodiments 1-17, wherein R³ is C₁-C₆ alkyl.
   22. The compound of any one of Embodiments 1-17, wherein R³ is C₁-C₄ alkyl.
   23. The compound of any one of Embodiments 1-17, wherein R³ is hydroxy.
   24. A compound having the structure of formula M-IV:

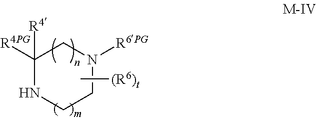

or a salt thereof, wherein:
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 0, 1, or 2;
n is 1, or 2;
p is 0, 1, or 2;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   25. A compound having the structure of formula M-VI:

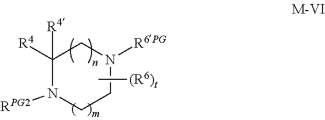

or a salt thereof, wherein:
m is 0, 1, or 2;
n is 1, or 2;
R^{PG2} is an amino protecting group;
R⁴ is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl;
R⁵ is —C(O)R⁸, —CH₂C(O)R¹, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
p is 0, 1, or 2;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   26. A compound having the structure of formula M-VII:

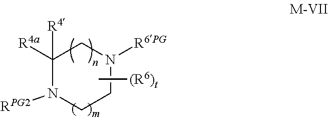

or a salt thereof, wherein:
m is 0, 1, or 2;
n is 1, or 2;
R^{PG2} is an amino protecting group;
R^{4a} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5a} and further substituted with 0 or 1 substituent selected from halogen, C₁-C₄ alkyl, C₁-C₄ alkoxy, hydroxy C₁-C₄ alkyl, hydroxy, and cyanomethyl;
R^{5a} is —CHO, —CH₂CHO, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
p is 0, 1, or 2;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   27. A compound having the structure of formula M-VIII:

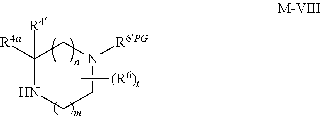

or a salt thereof, wherein:
m is 0, 1, or 2;
n is 1, or 2;
R^{4a} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5a} and further substituted with 0 or 1 substituent selected from halogen, C₁-C₄ alkyl, C₁-C₄ alkoxy, hydroxy C₁-C₄ alkyl, hydroxy, and cyanomethyl;
R^{5a} is —CHO, —CH₂CHO, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
p is 0, 1, or 2;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   28. A compound having the structure of formula M-IX:

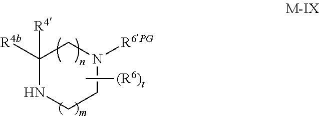

or a salt thereof, wherein:
m is 0, 1, or 2;
n is 1, or 2;
R^{4b} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5b} and further substituted with 0 or 1 substituent selected from halogen, C₁-C₄ alkyl, C₁-C₄ alkoxy, hydroxy C₁-C₄ alkyl, hydroxy, and cyanomethyl;
R^{5b} is —CH₂OH, —CH₂ CH₂OH, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
R^{4′} is hydrogen, C₁-C₄ alkyl, or hydroxy C₁-C₄ alkyl;
p is 0, 1, or 2;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   29. A compound having the structure of formula M-X:

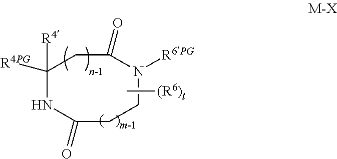

or a salt thereof, wherein:
m is 1, or 2;
n is 1, or 2;
R^{4′} is hydrogen, C₁-C₄ alkyl, or hydroxy C₁-C₄ alkyl;
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   30. A compound having the structure of formula M-XI:

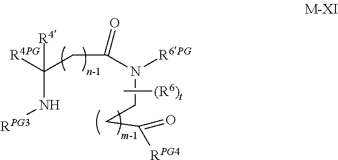

or a salt thereof, wherein:
m is 1, or 2;
n is 1, or 2;
R^{PG3} is —H or an amino protecting group;
R^{PG4} is —OH or —C(O)R^{PG4} is a protected carboxyl group;
R^{4′} is hydrogen, C₁-C₄ alkyl, or hydroxy C₁-C₄ alkyl;
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   31. A compound having the structure of formula M-XII:

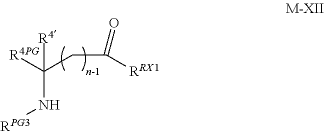

or a salt thereof, wherein:
n is 1, or 2;
R^{PG3} is —H or an amino protecting group;
R^{4′} is hydrogen, C₁-C₄ alkyl, or hydroxy C₁-C₄ alkyl;
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R⁵PG and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2;
R^{RX1} is —OH or —C(O)R^{RX1} is an activated carboxyl group;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl; and
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups.
   32. A compound having the structure of formula M-XIII:

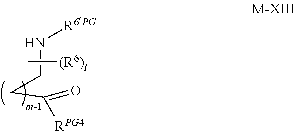

or a salt thereof, wherein:
m is 1, or 2;
R^{PG4} is —OH or —C(O)R^{PG4} is a protected carboxyl group;
R^{6′PG} is R^{6′} or an amino protecting group;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   33. A compound having the structure of formula M-XIV:

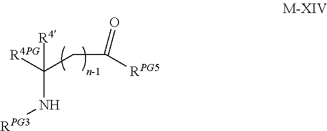

or a salt thereof, wherein:
n is 1, or 2;
R^{PG3} is —H or an amino protecting group;
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2; —C(O)R^{PG5} is protected carboxyl;
R^{4′} is hydrogen, C₁-C₄ alkyl, or hydroxy C₁-C₄ alkyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl; and
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups.
   34. A compound having the structure of formula M-IV′:

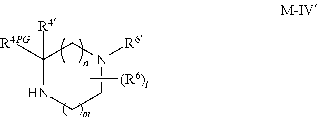

or a salt thereof, wherein:
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2;
m is 0, 1, or 2;
n is 1, or 2;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   35. A compound having the structure of formula M-XV:

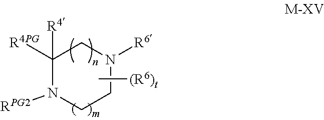

or a salt thereof, wherein:
R^{PG2} is an amino protecting group;
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 0, 1, or 2;
n is 1, or 2;
p is 0, 1, or 2;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   36. A compound having the structure of formula M-XVI:

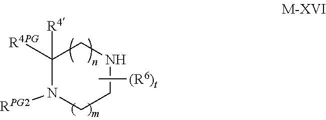

or a salt thereof, wherein:
R^{PG2} is an amino protecting group;
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 0, 1, or 2;
n is 1, or 2;
p is 0, 1, or 2;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl; and
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups.
   37. A compound having the structure of formula M-XVII:

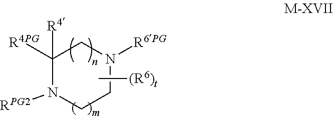

or a salt thereof, wherein:
R^{PG2} is an amino protecting group;
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 0, 1, or 2;
n is 1, or 2;
p is 0, 1, or 2;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
   38. The compound of any one of the preceding Embodiments, wherein R⁴ is phenyl, naphthyl or 5- or 6-membered heteroaryl, wherein the phenyl, naphthyl or 5- or 6-membered heteroaryl is substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl
   39. The compound of any one of the preceding Embodiments, wherein R⁴ is phenyl substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl.
   40. The compound of any one of the preceding Embodiments, wherein R⁴ is phenyl para-substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl.
   41. The compound of any one of the preceding Embodiments, wherein R⁴ is phenyl meta-substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl.
   42. The compound of any one of Embodiments 1-38, wherein R⁴ is naphthyl substituted with R⁵ and further substituted with 0 or 1 substituent selected from halogen, C₁-C₄ alkyl, C1-C4 alkoxy, hydroxy C₁-C₄ alkyl, hydroxy, and cyanomethyl.
   43. The compound of any one of Embodiments 1-38, wherein R⁴ is 5- or 6-membered heteroaryl substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl.
   44. The compound of any one of Embodiments 1-38, wherein R⁴ is 5-membered heteroaryl substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl.
   45. The compound of any one of Embodiments 1-38, wherein R⁴ is 6-membered heteroaryl substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl.
   46. The compound of any one of Embodiments 1-38, wherein R⁴ is 6-membered heteroaryl para-substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl.
   47. The compound of any one of Embodiments 1-38, wherein R⁴ is 6-membered heteroaryl meta-substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl.
   48. The compound of any one of the preceding Embodiments, wherein R⁴ is phenyl, naphthyl or 5- or 6-membered heteroaryl, wherein the phenyl, naphthyl or 5- or 6-membered heteroaryl is substituted with R⁵ and without further substitution.
   49. The compound of any one of Embodiments 1-38, wherein R⁴ is

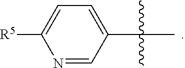

50. The compound of any one of Embodiments 1-38, wherein R⁴ is

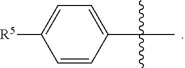

51. The compound of any one of Embodiments 1-38, wherein R⁴ is

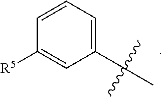

52. The compound of any one of Embodiments 1-38, wherein R⁴ is phenyl para-substituted with R⁵, and optionally substituted with —F, —OCH₃, —CH₂OH or —OH.
53. The compound of any one of Embodiments 1-38, wherein R⁴ is phenyl meta-substituted with R⁵, and optionally substituted with —F, —OCH₃, —CH₂OH or —OH.
54. The compound of any one of the preceding Embodiments, wherein R⁵ is —C(O)R⁸.
55. The compound of any one of the preceding Embodiments, wherein R⁵ is —CH₂C(O)R⁸.
56. The compound of any one of the preceding Embodiments, wherein R⁸ is hydroxy.
57. The compound of any one of the preceding Embodiments, wherein R⁸ is C₁-C₄ alkoxy.
58. The compound of any one of the preceding Embodiments, wherein R⁸ is —NH₂.
59. The compound of any one of the preceding Embodiments, wherein R⁸ is 5-7 membered saturated heterocyclyl having 1, 2, or 3 ring heteroatoms independently selected from nitrogen, oxygen and sulfur.
60. The compound of any one of the preceding Embodiments, wherein R⁸ is mono- and di-C₁-C₄ alkylamino which is optionally substituted with one or more substituents independently selected from halogen, hydroxy and C₁-C₄ alkyl.
61. The compound of any one of Embodiments 1-53, wherein R⁵ is —C(O)NHSO₂C₁-C₄alkyl.
62. The compound of any one of Embodiments 1-53, wherein R⁵ is —SO₂NHC(O)C₁-C₄alkyl.
63. The compound of any one of Embodiments 1-53, wherein R⁵ is —SO₂N(H)ₚ(C₁-C₄alkyl)₂₋ₚ.
64. The compound of any one of Embodiments 1-53, wherein R⁵ is —SO₂NH₂.
65. The compound of any one of Embodiments 1-53, wherein R⁵ is —SO(NH)C₁-C₄alkyl.
66. The compound of any one of Embodiments 1-53, wherein R⁵ is —SO(NH)CH₃.
67. The compound of any one of Embodiments 1-53, wherein R⁵ is —SO₂C₁-C₄alkyl.
68. The compound of any one of Embodiments 1-53, wherein R⁵ is cyano.
69. The compound of any one of Embodiments 1-53, wherein R⁵ is halogen.
70. The compound of any one of Embodiments 1-53, wherein R⁵ is hydroxy C₁-C₄ alkyl.
71. The compound of any one of Embodiments 1-53, wherein R⁵ is —B(R′)₂.
72. The compound of Embodiment 71, wherein each R⁸ is independently hydroxy or C₁-C₄ alkoxy.
73. The compound of Embodiment 71, wherein each R⁸ is independently hydroxy.
74. The compound of Embodiment 71, wherein each R⁸ is independently C₁-C₄ alkoxy.
75. The compound of any one of Embodiments 1-53, wherein R⁵ is 5-membered heteroaryl having 1-4 ring nitrogen atoms and 0 or 1 ring oxygen or sulfur atom.
76. The compound of Embodiment 75, wherein R⁵ is tetrazolyl.
77. The compound of any one of the preceding Embodiments, wherein R^{4′} is hydrogen.
78. The compound of any one of the preceding Embodiments, wherein t is 1, 2, 3 or 4.
79. The compound of any one of the preceding Embodiments, wherein an occurrence of R⁶ is hydroxy.
80. The compound of any one of the preceding Embodiments, wherein an occurrence of R⁶ is amino.
81. The compound of any one of the preceding Embodiments, wherein an occurrence of R⁶ is mono- and di-C₁-C₄ alkylamino.
82. The compound of any one of the preceding Embodiments, wherein an occurrence of R⁶ is C₁-C₆ alkyl.
83. The compound of any one of the preceding Embodiments, wherein an occurrence of R⁶ is C₁-C₄ alkyl.
84. The compound of any one of the preceding Embodiments, wherein an occurrence of R⁶ is methyl.
85. The compound of any one of the preceding Embodiments, wherein an occurrence of R⁶ is halo C₁-C₆ alkyl.
86. The compound of any one of the preceding Embodiments, wherein an occurrence of R⁶ is halo C₁-C₄ alkyl.
87. The compound of any one of the preceding Embodiments, wherein an occurrence of R⁶ is —CH₂F.
88. The compound of any one of the preceding Embodiments, wherein an occurrence of R⁶ is hydroxy C₁-C₆ alkyl.
89. The compound of any one of the preceding Embodiments, wherein an occurrence of R⁶ is hydroxy C₁-C₄ alkyl.
90. The compound of any one of the preceding Embodiments, wherein an occurrence of R⁶ is —CH₂OH.
91. The compound of any one of the preceding Embodiments, wherein an occurrence of R⁶ is cyano C₁-C₆ alkyl.
92. The compound of any one of the preceding Embodiments, wherein an occurrence of R⁶ is cyano C₁-C₄ alkyl.
93. The compound of any one of the preceding Embodiments, wherein an occurrence of R⁶ is C₁-C₆ alkoxy.
94. The compound of any one of the preceding Embodiments, wherein an occurrence of R⁶ is C₁-C₄ alkoxy.
95. The compound of any one of the preceding Embodiments, wherein two R⁶ on a carbon atom are taken together to form ═O.
96. The compound of any one of the preceding Embodiments, wherein two R⁶ on a carbon atom are taken together to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl.
97. The compound of any one of the preceding Embodiments, wherein two R⁶ on a carbon atom are taken together to form a cyclopropyl ring.
98. The compound of any one of the preceding Embodiments, wherein two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl.
99. The compound of any one of the preceding Embodiments, wherein two R⁶ are taken together with their intervening atoms to form a 5-7 membered cycloalkyl ring, wherein the ring is optionally substituted with one or more substituents independently selected from halogen, C₁-C₄ alkyl, halo C₁-C₄ alkyl, C₁-C₄ alkoxy, hydroxyl, and carbonyl.
100. The Embodiment of any one of the preceding Embodiments, wherein m is 0.
101. The compound of any one of Embodiments 1-99, wherein m is 1.
102. The compound of any one of Embodiments 1-99, wherein m is 2.
103. The Embodiment of any one of the preceding Embodiments, wherein n is 0.
104. The compound of any one of Embodiments 1-102, wherein n is 1.
105. The compound of any one of Embodiments 1-102, wherein n is 2.
106. The compound of any one of Embodiments 1-99, wherein the compound has the structure of:

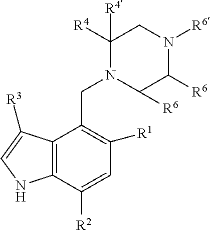

or a pharmaceutically acceptable salt thereof.
107. The compound of Embodiment 106, wherein an occurrence of R⁶ is hydrogen.
108. The compound of any one of Embodiments 106-107, wherein each R⁶ is independently selected from —H, —CH₂OH, —CH₂F, —CHF₂ and —CF₃.
109. The compound of any one of Embodiments 1-94, wherein the compound has the structure of:

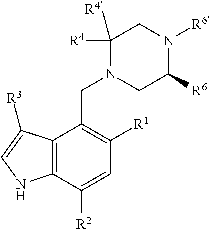

or a pharmaceutically acceptable salt thereof.
110. The compound of any one of Embodiments 1-94, wherein the compound has the structure of:

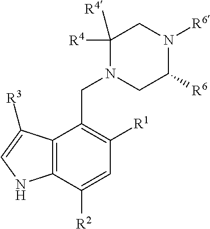

or a pharmaceutically acceptable salt thereof.
111. The compound of any one of Embodiments 1-99, wherein the compound has the structure of:

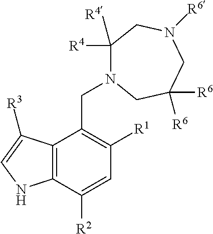

or a pharmaceutically acceptable salt thereof.
112. The compound of any one of Embodiments 1-99, wherein the compound has the structure of:

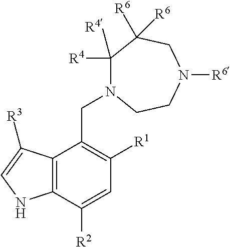

or a pharmaceutically acceptable salt thereof.
113. The compound of any one of Embodiments 111-112, wherein an occurrence of R⁶ is hydrogen.
114. The compound of any one of Embodiments 111-113, wherein an occurrence of R⁶ is —OH.
115. The compound of any one of Embodiments 111-113, wherein an occurrence of R⁶ is halogen.
116. The compound of any one of Embodiments 111-113, wherein an occurrence of R⁶ is —F.
117. The compound of any one of Embodiments 111-113, wherein an occurrence of R⁶ is C₁₋₆ alkyl.
118. The compound of any one of Embodiments 111-113, wherein an occurrence of R⁶ is methyl.
119. The compound of any one of Embodiments 111-112, wherein each R⁶ is independently halogen.
120. The compound of any one of Embodiments 111-112, wherein each R⁶ is independently —F.
121. The compound of any one of Embodiments 1-99, wherein t is 2, 3 or 4.
122. The compound of any one of Embodiments 1-99, wherein t is 2.
123. The compound of any one of the preceding Embodiments, wherein at least one occurrence of R⁶ is bonded to a carbon atom that is bonded to the nitrogen atom to which R^{6′} is bonded to.
124. The compound of Embodiment 123, wherein the carbon atom is not bonded to the carbon atom to which R⁴ is bonded to.
125. The compound of Embodiment 123, wherein the carbon atom is bonded to the carbon atom to which R⁴ is bonded to.
126. The compound of any one of Embodiments 1-77, wherein t is 0.
127. The compound of any one of Embodiments 1-94, wherein t is 1.
128. The compound of any one of Embodiments 1-77, wherein the compound has the structure of:

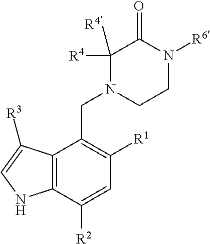

or a pharmaceutically acceptable salt thereof.
129. The compound of any one of Embodiments 1-77, wherein the compound has the structure of:

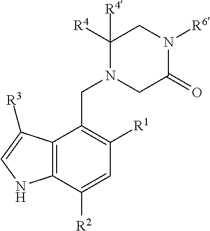

or a pharmaceutically acceptable salt thereof.
130. The compound of any one of Embodiments 1-77, wherein the compound has the structure of:

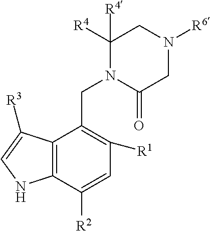

or a pharmaceutically acceptable salt thereof.
131. The compound of any one of Embodiments 1-94, wherein the compound has the structure of:

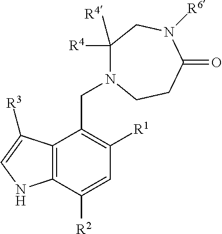

or a pharmaceutically acceptable salt thereof.
132. The compound of any one of Embodiments 1-94, wherein the compound has the structure of:

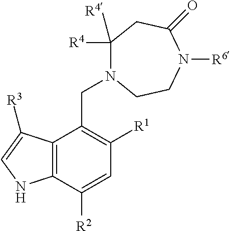

or a pharmaceutically acceptable salt thereof.
133. The compound of any one of the preceding Embodiments, wherein R^{6′} is hydrogen.
134. The compound of any one of Embodiments 1-132, wherein R^{6′} is —CH₂-(hydroxy C₁-C₄ alkyl).
135. The compound of any one of Embodiments 1-132, wherein R^{6′} is —CH₂—CH₂OH.
136. The compound of any one of Embodiments 1-132, wherein R^{6′} is —CH₂-(amino C₁-C₄ alkyl).
137. The compound of any one of Embodiments 1-132, wherein R^{6′} is —CH₂-(mono- and di-C₁-C₄ alkylamino C₁-C₄ alkyl).
138. The compound of any one of Embodiments 1-132, wherein R^{6′} is C₁-C₆ aliphatic.
139. The compound of any one of Embodiments 1-132, wherein R^{6′} is C₁-C₆ linear aliphatic.
140. The compound of any one of Embodiments 1-139, wherein R^{6′} is C₁-C₆ alkyl.
141. The compound of any one of Embodiments 1-139, wherein R^{6′} is C₁-C₄ alkyl.
142. The compound of any one of Embodiments 1-132, wherein R^{6′} is methyl.
143. The compound of any one of Embodiments 1-132, wherein R^{6′} is ethyl.
144. The compound of any one of Embodiments 1-132, wherein R^{6′} is n-propyl.
145. The compound of any one of Embodiments 1-132, wherein R^{6′} is isopropyl.
146. The compound of any one of Embodiments 1-132, wherein R^{6′} is n-butyl.
147. The compound of any one of Embodiments 1-132, wherein R^{6′} is 2-butyl.
148. The compound of any one of Embodiments 1-132, wherein R^{6′} is —CH₂CH═CH₂.
149. The compound of any one of Embodiments 1-132, wherein R^{6′} is —CH₂CH—CH₂.
150. The compound of any one of Embodiments 1-132, wherein R^{6′} is C₁-C₄ alkoxy.
151. The compound of any one of Embodiments 1-132, wherein R^{6′} is C₃-C₆ cycloalkyl.
152. The compound of any one of Embodiments 1-132, wherein R^{6′} is —CH(CH₃)—C₁-C₄ aliphatic.
153. The compound of Embodiment 152, wherein the C₁-C₄ aliphatic is linear.
154. The compound of Embodiment 152, wherein the C₁-C₄ aliphatic is C₁-C₄ alkyl.
155. The compound of Embodiment 152, wherein the C₁-C₄ aliphatic is linear C₁-C₄ alkyl.
156. The compound of Embodiment 152, wherein the C₁-C₄ aliphatic is methyl.
157. The compound of Embodiment 152, wherein the C₁-C₄ aliphatic is ethyl.
158. The compound of Embodiment 152, wherein the C₁-C₄ aliphatic is n-propyl.
159. The compound of any one of Embodiments 1-132, wherein R^{6′} is C₃-C₆ cycloaliphatic.
160. The compound of any one of Embodiments 1-132, wherein R^{6′} is C₃-C₆ cycloalkyl.
161. The compound of any one of Embodiments 1-132, wherein R^{6′} is cyclopropyl.
162. The compound of any one of Embodiments 1-132, wherein R^{6′} is cyclobutyl.
163. The compound of any one of Embodiments 1-132, wherein R^{6′} is cyclopentyl.
164. The compound of any one of Embodiments 1-132, wherein R^{6′} is —CH₂—C₃-C₅ cycloaliphatic.
165. The compound of Embodiment 164, wherein the C₃-C₅ cycloaliphatic is C₃-C₅ cycloalkyl.
166. The compound of Embodiment 164, wherein the C₃-C₅ cycloaliphatic is cyclopropyl.
167. The compound of Embodiment 164, wherein the C₃-C₅ cycloaliphatic is cyclobutyl.
168. The compound of Embodiment 164, wherein the C₃-C₅ cycloaliphatic is cyclopentyl.
169. The compound of any one of Embodiments 1-132, wherein R^{6′} is C₁-C₆ aliphatic substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl.
170. The compound of any one of Embodiments 1-132, wherein R^{6′} is C₁-C₆ alkyl substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl.
171. The compound of any one of Embodiments 169-170, wherein R^{6′} is C₁-C₆ aliphatic substituted with one or more halogen.
172. The compound of any one of Embodiments 169-171, wherein R^{6′} is C₁-C₆ alkyl substituted with one or more —F.
173. The compound of any one of Embodiments 169-171, wherein R^{6′} is C₁-C₆ linear alkyl substituted with one or more —F.
174. The compound of any one of Embodiments 169-173, wherein R^{6′} is —CH₂CH₂F.
175. The compound of any one of Embodiments 169-173, wherein R^{6′} is —CH₂CH₂CH₂F.
176. The compound of any one of Embodiments 169-173, wherein R^{6′} is —CH₂CHF₂.
177. The compound of any one of Embodiments 169-173, wherein R^{6′} is —CH₂CH₂CHF₂.
178. The compound of any one of Embodiments 169-173, wherein R^{6′} is —CH₂CF₃.
179. The compound of any one of Embodiments 169-172, wherein R^{6′} is —CH(CH₃)—CHF₂.
180. The compound of any one of Embodiments 169-173, wherein R^{6′} is —CH₂—CF₂—CH₃.
181. The compound of any one of Embodiments 169-172, wherein R^{6′} is —CH(CH₃)—CH₂—CF₃.
182. The compound of any one of Embodiments 169-173, wherein R^{6′} is —CH₂CH₂CF₃.
183. The compound of any one of Embodiments 169-171, wherein R^{6′} is —CH₂CF═CH₂.
184. The compound of any one of Embodiments 169-170, wherein R^{6′} is C₁-C₆ aliphatic substituted with one or more C₁-C₄ alkyl wherein the alkyl is optionally and independently substituted with one or more substituents independently selected from halogen, C₁-C₄ alkyl, halo C₁-C₄ alkyl, C₁-C₄ alkoxy, hydroxyl, and carbonyl.
185. The compound of any one of Embodiments 169-170, wherein R^{6′} is C₁-C₆ aliphatic substituted with one or more C₁-C₄ alkyl.
186. The compound of any one of Embodiments 169-171, wherein R^{6′} is C₁-C₆ aliphatic substituted with one or more C₁-C₄ alkoxy wherein the alkoxy is optionally and independently substituted with one or more substituents independently selected from halogen, C₁-C₄ alkyl, halo C₁-C₄ alkyl, C₁-C₄ alkoxy, hydroxyl, and carbonyl.
187. The compound of any one of Embodiments 169-171, wherein R^{6′} is C₁-C₆ aliphatic substituted with one or more C₁-C₄ alkoxy.
188. The compound of any one of Embodiments 186-187, wherein an occurrence of C₁-C₄ alkoxy is methoxy.
189. The compound of any one of Embodiments 169-171, wherein R^{6′} is C₁-C₆ aliphatic substituted with one or more C₃-C₆ cycloalkyl wherein the cycloalkyl is optionally and independently substituted with one or more substituents independently selected from halogen, C₁-C₄ alkyl, halo C₁-C₄ alkyl, C₁-C₄ alkoxy, hydroxyl, and carbonyl.
190. The compound of any one of Embodiments 169-171, wherein R^{6′} is C₁-C₆ aliphatic substituted with one or more C₃-C₆ cycloalkyl.
191. The compound of any one of Embodiments 169-171, wherein R^{6′} is C₁-C₆ aliphatic substituted with one or more 3-6 membered heterocyclyl having 1-4 ring atoms independently selected from nitrogen, oxygen and sulfur, wherein the heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl.
192. The compound of any one of Embodiments 169-171, wherein R^{6′} is C₁-C₆ aliphatic substituted with one or more 3-6 membered heterocyclyl having 1-4 ring atoms independently selected from nitrogen, oxygen and sulfur, wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl.
193. The compound of any one of Embodiments 191-192, wherein an occurrence of 3-6 membered heterocyclyl having 1-4 ring atoms independently selected from nitrogen, oxygen and sulfur is.

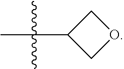 [SMILES: *C1COC1]

194. The compound of any one of Embodiments 184-193, wherein the C₁-C₆ aliphatic is linear.
195. The compound of any one of Embodiments 184-194, wherein the C₁-C₆ aliphatic is C₁-C₆ alkyl.
196. The compound of any one of Embodiments 184-195, wherein the C₁-C₆ aliphatic is ethyl.
197. The compound of Embodiment 196, wherein R^{6′} is —CH₂CH₂OCH₃.
198. The compound of any one of Embodiments 184-195, wherein the C₁-C₆ aliphatic is methyl.
199. The compound of Embodiment 198, wherein R^{6′} is

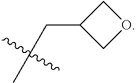 [SMILES: *CC1COC1]

200. The compound of any one of Embodiments 1-132, wherein R^{6′} is R¹⁰.
201. The compound of Embodiment 200, wherein R¹⁰ is 5-6 membered heteroaryl having a nitrogen ring atom, wherein the heteroaryl is optionally substituted with one or more substituents independently selected from halogen, C₁-C₄ alkyl, halo C₁-C₄ alkyl, C₁-C₄ alkoxy, hydroxyl, and carbonyl, and wherein the nitrogen ring atom of the heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
202. The compound of Embodiment 200, wherein R¹⁰ is 5-6 membered heteroaryl having a nitrogen ring atom, wherein the nitrogen ring atom of the heteroaryl is substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
203. The compound of Embodiment 200, wherein R¹⁰ is 3-6 membered heterocyclyl having a nitrogen ring atom, wherein the heterocyclyl is optionally substituted with one or more substituents independently selected from halogen, C₁-C₄ alkyl, halo C₁-C₄ alkyl, C₁-C₄ alkoxy, hydroxyl, and carbonyl, and wherein the nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
204. The compound of Embodiment 200, wherein R¹⁰ is 3-6 membered heterocyclyl having a nitrogen ring atom, wherein the nitrogen ring atom of the heterocyclyl is substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.
205. The compound of Embodiment 200, wherein R¹⁰ is 3-6 membered heterocyclyl having a nitrogen ring atom substituted with C₁₋₃ acyl.
206. The compound of Embodiment 200, wherein R¹⁰ is

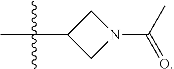 [SMILES: *C1CN(C(C)=O)C1]

207. The compound of any one of Embodiments 1-122, wherein R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl.
208. The compound of any one of Embodiments 1-122, wherein R⁶ and R^{6′} are taken together with their intervening atoms to form a 5-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl.
209. The compound of any one of Embodiments 1-122, wherein R⁶ and R^{6′} are taken together with their intervening atoms to form a 5-8 membered monocyclic saturated or partially unsaturated ring having 1 ring nitrogen atom.
210. The compound of any one of Embodiments 1-122, wherein R⁶ and R^{6′} are taken together with their intervening atoms to form a 5-8 membered monocyclic saturated ring having 1 ring nitrogen atom.
211. The compound of any one of Embodiments 1-122, wherein R⁶ and R^{6′} are taken together with their intervening atoms to form a 5-membered monocyclic saturated ring having 1 ring nitrogen atom.
212. The compound of any one of the preceding Embodiments, wherein the compound has the structure of:

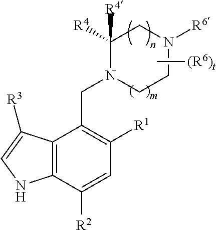

or a pharmaceutically acceptable salt thereof.
213. The compound of any one of the preceding Embodiments, wherein the compound has the structure of:

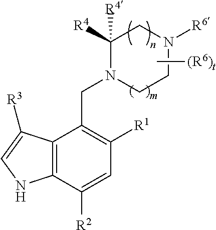

or a pharmaceutically acceptable salt thereof.
214. The compound of any one of the preceding Embodiments, wherein R^{PG} is an amino protecting group.
215. The compound of any one of the preceding Embodiments, wherein R^{PG} is —C(O)-optionally substituted C₁-C₆ aliphatic.
216. The compound of any one of the preceding Embodiments, wherein R^{PG} is —Boc.
217. The compound of any one of the preceding Embodiments, wherein R^{PG}2 is an amino protecting group.
218. The compound of any one of the preceding Embodiments, wherein R^{PG}2 is —C(O)-optionally substituted C₁-C₆ aliphatic.
219. The compound of any one of the preceding Embodiments, wherein R^{PG}2 is —Boc.
220. The compound of any one of the preceding Embodiments, wherein R^{PG3} is an amino protecting group.
221. The compound of any one of the preceding Embodiments, wherein R^{PG3} is —C(O)-optionally substituted C₁-C₆ aliphatic.
222. The compound of any one of the preceding Embodiments, wherein R^{PG3} is —Boc.
223. The compound of any one of the preceding Embodiments, wherein —C(O)R^{PG4} is a protected carboxyl group.
224. The compound of any one of the preceding Embodiments, wherein R^{PG4} is —O-optionally substituted C₁-C₆ aliphatic.
225. The compound of any one of the preceding Embodiments, wherein R^{PG4} is —O-optionally substituted C₁-C₆ alkyl.
226. The compound of any one of the preceding Embodiments, wherein R^{PG4} is —OMe.
227. The compound of any one of Embodiments 1-222, wherein R^{PG4} is —OH.
228. The compound of any one of the preceding Embodiments, wherein —C(O)R^{PG5} is a protected carboxyl group.
229. The compound of any one of the preceding Embodiments, wherein R^{PG5} is —O-optionally substituted C₁-C₆ aliphatic.
230. The compound of any one of the preceding Embodiments, wherein R^{PG5} is —O-optionally substituted C₁-C₆ alkyl.
231. The compound of any one of the preceding Embodiments, wherein R^{PG5} is —OMe.
232. The compound of any one of Embodiments 1-227, wherein R^{PG5} is —OH.
233. The compound of any one of the preceding Embodiments, wherein R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl.
234. The compound of any one of the preceding Embodiments, wherein R^{4PG} is phenyl substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl.
235. The compound of any one of the preceding Embodiments, wherein R^{4PG} is phenyl substituted with R^{5PG}.
236. The compound of any one of the preceding Embodiments, wherein R^{4PG} is

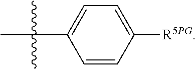

237. The compound of any one of the preceding Embodiments, wherein R^{5PG} is R^{5PG} is —C(O)R^{8PG}, wherein R^{8PG} is optionally substituted C₁₋₆ aliphatic.
238. The compound of any one of the preceding Embodiments, wherein R^{5PG} is R^{5PG} is —C(O)CH₃.
239. The compound of any one of the preceding Embodiments, wherein R^{4a} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is substituted with R^{5a} and further substituted with 0 or 1 substituent selected from halogen, C₁-C₄ alkyl, C₁-C₄ alkoxy, hydroxy C₁-C₄ alkyl, hydroxy, and cyanomethyl.
240. The compound of any one of the preceding Embodiments, wherein R^{4a} is phenyl substituted with R^{5a} and further substituted with 0 or 1 substituent selected from halogen, C₁-C₄ alkyl, C₁-C₄ alkoxy, hydroxy C₁-C₄ alkyl, hydroxy, and cyanomethyl.
241. The compound of any one of the preceding Embodiments, wherein R^{4a} is phenyl substituted with R^{5a}.
242. The compound of any one of the preceding Embodiments, wherein R⁴ is

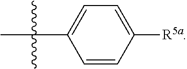

243. The compound of any one of the preceding Embodiments, wherein R^{4a} is —CHO.
244. The compound of any one of the preceding Embodiments, wherein R^{4a} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is substituted with R^{5b} and further substituted with 0 or 1 substituent selected from halogen, C₁-C₄ alkyl, C₁-C₄ alkoxy, hydroxy C₁-C₄ alkyl, hydroxy, and cyanomethyl.
245. The compound of any one of the preceding Embodiments, wherein R^{4b} is phenyl substituted with R^{5b} and further substituted with 0 or 1 substituent selected from halogen, C₁-C₄ alkyl, C₁-C₄ alkoxy, hydroxy C₁-C₄ alkyl, hydroxy, and cyanomethyl.
246. The compound of any one of the preceding Embodiments, wherein R^{4b} is phenyl substituted with R^{5b}.
247. The compound of any one of the preceding Embodiments, wherein R^{4b} is

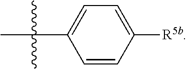

248. The compound of any one of the preceding Embodiments, wherein R^{5b} is —CH₂OH.
249. The compound of any one of the preceding Embodiments, wherein R^{6′PG} is an amino protecting group.
250. The compound of any one of the preceding Embodiments, wherein R^{6′PG} is —C(O)-optionally substituted C₁-C₆ aliphatic.
251. The compound of any one of the preceding Embodiments, wherein R^{6′PG} is —Boc.
252. The compound of any one of Embodiments 1-248, wherein R^{6′PG} is —Bn.
253. The compound of any one of the preceding Embodiments, wherein —C(O)R¹ is an activated carboxyl group.
254. The compound of any one of Embodiments 1-252, wherein R^{RX1} is —OH.
255. A compound selected from Table El or a pharmaceutically acceptable salt thereof.
256. A compound having the structure of

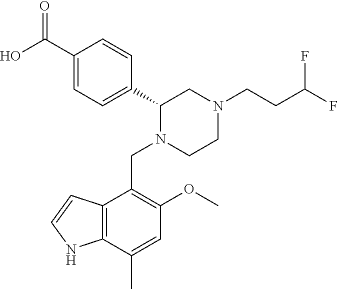 [SMILES: COc1cc(C)c2[nH]ccc2c1CN1CCN(CCC(F)F)C[C@H]1c1ccc(C(=O)O)cc1]

or a pharmaceutically acceptable salt thereof.
257. A compound having the structure of

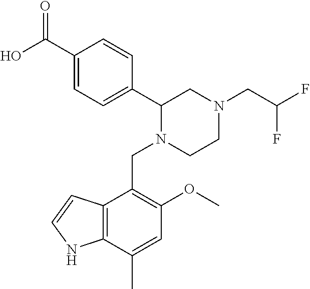 [SMILES: COc1cc(C)c2[nH]ccc2c1CN1CCN(CC(F)F)CC1c1ccc(C(=O)O)cc1]

or a pharmaceutically acceptable salt thereof.
258. A compound having the structure of

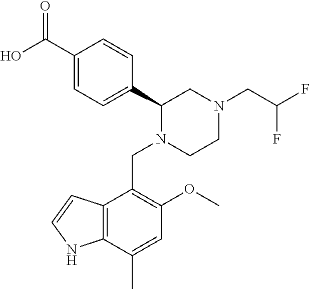 [SMILES: COc1cc(C)c2[nH]ccc2c1CN1CCN(CC(F)F)C[C@@H]1c1ccc(C(=O)O)cc1]

or a pharmaceutically acceptable salt thereof.
259. A compound having the structure of

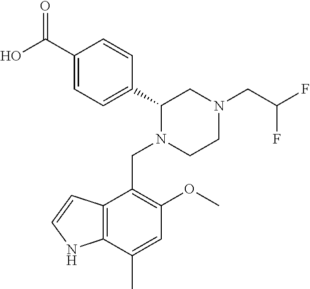 [SMILES: COc1cc(C)c2[nH]ccc2c1CN1CCN(CC(F)F)C[C@H]1c1ccc(C(=O)O)cc1]

or a pharmaceutically acceptable salt thereof.
260. A compound having the structure of

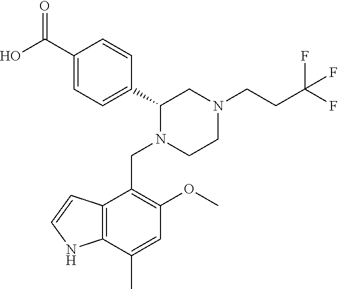 [SMILES: COc1cc(C)c2[nH]ccc2c1CN1CCN(CCC(F)(F)F)C[C@H]1c1ccc(C(=O)O)cc1]

or a pharmaceutically acceptable salt thereof.
261. A compound having the structure of

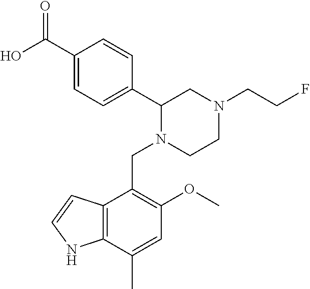 [SMILES: COc1cc(C)c2[nH]ccc2c1CN1CCN(CCF)CC1c1ccc(C(=O)O)cc1]

or a pharmaceutically acceptable salt thereof.
262. A compound having the structure of

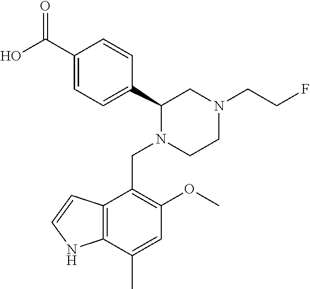 [SMILES: COc1cc(C)c2[nH]ccc2c1CN1CCN(CCF)C[C@@H]1c1ccc(C(=O)O)cc1]

or a pharmaceutically acceptable salt thereof.
263. A compound having the structure of

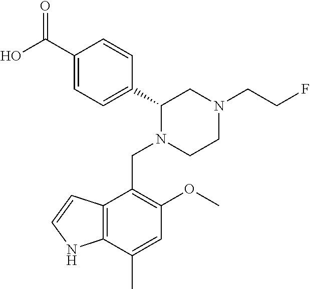 [SMILES: COc1cc(C)c2[nH]ccc2c1CN1CCN(CCF)C[C@H]1c1ccc(C(=O)O)cc1]

or a pharmaceutically acceptable salt thereof.
264. A compound having the structure of

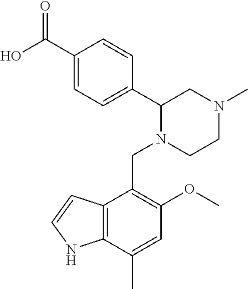 [SMILES: COc1cc(C)c2[nH]ccc2c1CN1CCN(C)CC1c1ccc(C(=O)O)cc1]

or a pharmaceutically acceptable salt thereof.
265. A compound having the structure of

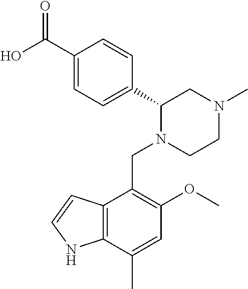 [SMILES: COc1cc(C)c2[nH]ccc2c1CN1CCN(C)C[C@H]1c1ccc(C(=O)O)cc1]

or a pharmaceutically acceptable salt thereof.
266. A compound having the structure of

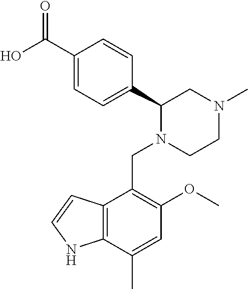 [SMILES: COc1cc(C)c2[nH]ccc2c1CN1CCN(C)C[C@@H]1c1ccc(C(=O)O)cc1]

or a pharmaceutically acceptable salt thereof.
267. A compound having the structure of

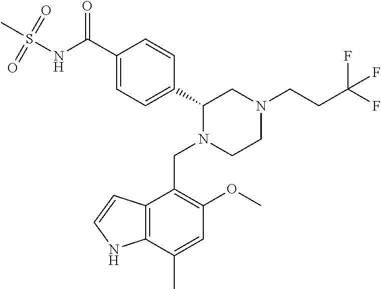 [SMILES: COc1cc(C)c2[nH]ccc2c1CN1CCN(CCC(F)(F)F)C[C@H]1c1ccc(C(=O)NS(C)(=O)=O)cc1]

or a pharmaceutically acceptable salt thereof.
268. A compound having the structure of

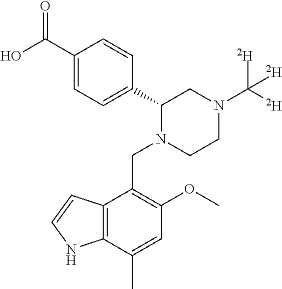 [SMILES: [2H]C([2H])([2H])N1CCN(Cc2c(OC)cc(C)c3[nH]ccc23)[C@H](c2ccc(C(=O)O)cc2)C1]

or a pharmaceutically acceptable salt thereof.
269. A compound having the structure of

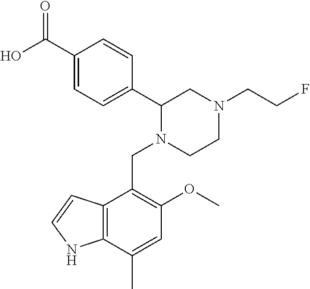 [SMILES: COc1cc(C)c2[nH]ccc2c1CN1CCN(CCF)CC1c1ccc(C(=O)O)cc1]

or a pharmaceutically acceptable salt thereof.
270. A compound having the structure of

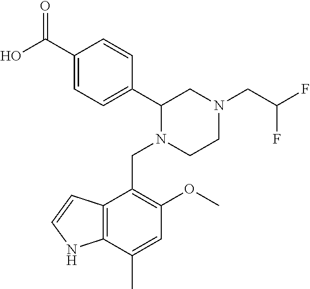 [SMILES: COc1cc(C)c2[nH]ccc2c1CN1CCN(CC(F)F)CC1c1ccc(C(=O)O)cc1]

or a pharmaceutically acceptable salt thereof.
271. A compound having the structure of

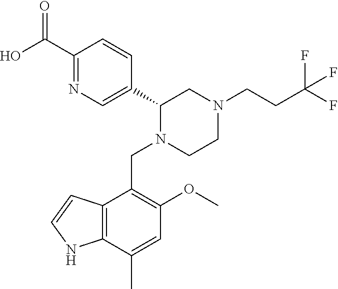 [SMILES: COc1cc(C)c2[nH]ccc2c1CN1CCN(CCC(F)(F)F)C[C@H]1c1ccc(C(=O)O)nc1]

or a pharmaceutically acceptable salt thereof.
272. A compound having the structure of

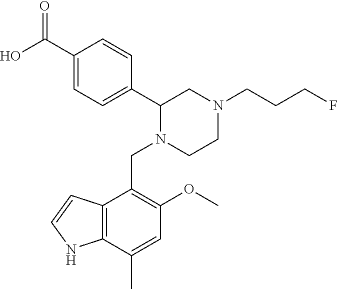 [SMILES: COc1cc(C)c2[nH]ccc2c1CN1CCN(CCCF)CC1c1ccc(C(=O)O)cc1]

or a pharmaceutically acceptable salt thereof.
273. A compound having the structure of

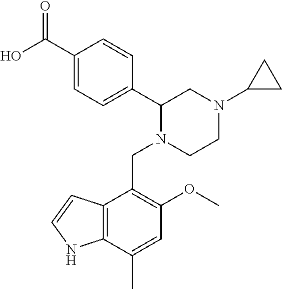 [SMILES: COc1cc(C)c2[nH]ccc2c1CN1CCN(C2CC2)CC1c1ccc(C(=O)O)cc1]

or a pharmaceutically acceptable salt thereof.
274. A compound having the structure of

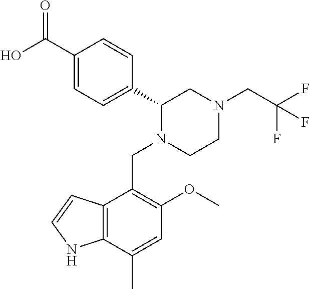 [SMILES: COc1cc(C)c2[nH]ccc2c1CN1CCN(CC(F)(F)F)C[C@H]1c1ccc(C(=O)O)cc1]

or a pharmaceutically acceptable salt thereof.
275. A compound having the structure of

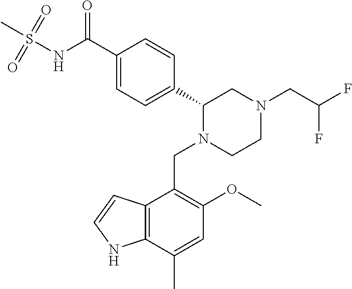 [SMILES: COc1cc(C)c2[nH]ccc2c1CN1CCN(CC(F)F)C[C@H]1c1ccc(C(=O)NS(C)(=O)=O)cc1]

or a pharmaceutically acceptable salt thereof.
276. The compound of any one of the preceding Embodiments, wherein the compound has an IC₅₀ of about or no more than about 1000, 900, 800, 700, 600, 500, 400, 300, 200, 100, 90, 80, 70, 60, 50, 40, 30, 20, or 10 nM as measured in an assay in the Examples.
277. The compound of any one of the preceding Embodiments, wherein the compound has a diastereomeric purity of about or no less than about 90%, 91%, 92%, 93%, 94%, 95%, 96%, 97%, 98%, or 99%.
278. The compound of any one of the preceding Embodiments, wherein the compound has a enantiomeric purity of about or no less than about 90%, 91%, 92%, 93%, 94%, 95%, 96%, 97%, 98%, or 99%.
279. The compound of any one of the preceding Embodiments, wherein the compound has a purity of about or no less than about 90%, 91%, 92%, 93%, 94%, 95%, 96%, 97%, 98%, or 99%.
280. A pharmaceutical composition comprising a compound of any one of the preceding Embodiments or a pharmaceutically acceptable salt thereof, and a pharmaceutically acceptable carrier, excipient or diluent.
281. A pharmaceutical composition which delivers a compound of any one of the preceding Embodiments or a pharmaceutically acceptable salt thereof.
282. A method, comprising removing one or more protection groups from a compound of formula M-I:

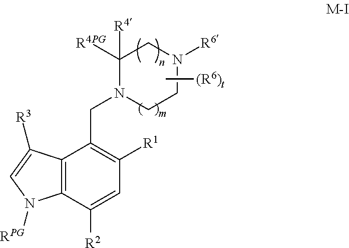

or a salt thereof to provide a compound of formula I or a salt thereof.
283. The method of Embodiment 282, wherein the compound of formula M-I or a salt thereof is a compound of any one of Embodiments 2-238.
284. The method of Embodiment 282 or 283, wherein the compound of formula I or a salt thereof is a compound of any one of Embodiments 1-211.
285. The method of any one of Embodiments 282-284, wherein R^{PG} is Boc.
286. The method of any one of Embodiments 282-285, wherein an ester in R^{4PG} is hydrolyzed to form —COOH or a salt form thereof.
287. A method comprising contacting an aldehyde or ketone with a compound of formula M-II:

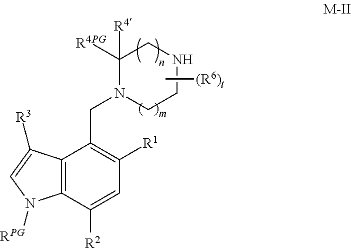

or a salt thereof in the presence of a reducing agent to provide a compound of formula M-I or a salt thereof.
288. The method of Embodiment 287, wherein the compound of formula M-I or a salt thereof is a compound of any one of Embodiments 2-238.
289. The method of Embodiment 287 or 288, wherein R^{6′} in formula M-I or a salt thereof is C₁-C₆ aliphatic, R¹⁰, —CH₂—C₃-C₆ cycloaliphatic, —CH₂—R¹⁰, —CH₂-(hydroxy C₁-C₄ alkyl), —CH₂-phenyl, —CH₂-(amino C₁-C₄ alkyl), or —CH₂-(mono- and di-C₁-C₄ alkylamino C₁-C₄ alkyl), each of which is independently optionally substituted.
290. The method of any one of Embodiments 287-289, wherein an aldehyde or ketone is of such a structure that when its —C(O)— group is replaced with —CH₂—, a compound of H—R^{6′} is formed.
291. The method of any one of Embodiments 287-290, wherein the reducing agent is triacetoxyborohydride.
292. The method of any one of Embodiments 287-291, wherein the compound of formula M-II or a salt thereof is a compound of any one of Embodiments 3-279.
293. The method of any one of Embodiments 282-286, comprising a method of any one of Embodiments 287-292.
294. A method comprising reacting a compound of formula M-II:

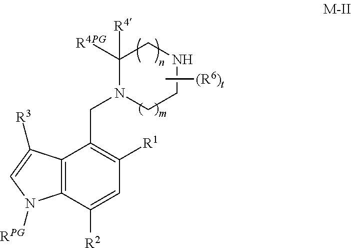

or a salt thereof with a compound having the structure of formula R^{6x3}—R^{6′}, wherein R^{6x3} is a leaving group.
295. The method of Embodiment 294, wherein the compound of formula M-II or a salt thereof is a compound of any one of Embodiments 3-279.
296. The method of Embodiment 294 or 295, wherein R^{6x3} is halogen.
297. The method of any one of Embodiments 294-296, wherein R^{6x3}—R^{6′} is bromobenzene.
298. The method of any one of Embodiments 294-296, wherein R^{6x3}—R^{6′} is bromobenzene.
299. The method of any one of Embodiments 294-296, wherein 1-fluoro-3-iodopropane.
300. The method of any one of Embodiments 282-286, comprising a method of any one of Embodiments 294-299.
301. A method comprising reacting a compound of formula M-II:

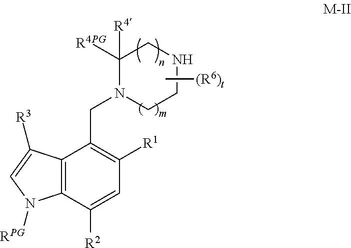

or a salt thereof with an acylating agent.
302. The method of Embodiment 301, wherein the compound of formula M-II or a salt thereof is a compound of any one of Embodiments 3-279.
303. The method of Embodiment 301 or 302, wherein an acylating agent has the structure of R^{6x1}—C(O)—C₁-C₆ aliphatic or a salt thereof, wherein R^{6x1} is —OH or R^{6x1}—C(O)— is an activated carboxyl group.
304. The method of any one of Embodiments 282-286, comprising a method of any one of Embodiments 301-303.
305. A method comprising reacting a compound of formula M-II:

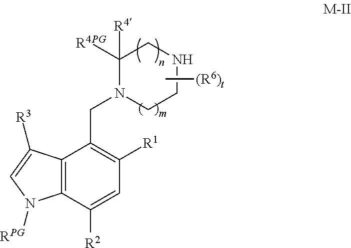

or a salt thereof with a sulfonylation agent.
306. The method of Embodiment 305, wherein the compound of formula M-II or a salt thereof is a compound of any one of Embodiments 3-279.
307. The method of Embodiment 305 or 306, wherein a sulfonylation agent has the structure of R^{6x2}—S(O)₂—C₁-C₆ aliphatic or a salt thereof, wherein R^{6x2}—S(O)₂— is an activated sulfonyl group.
308. The method of any one of Embodiments 305-307, wherein R^{6x2} is halogen.
309. The method of any one of Embodiments 305-307, wherein the sulfonylation agent is MsCl.
310. The method of any one of Embodiments 282-286, comprising a method of any one of Embodiments 305-309.
311. A method comprising de-protecting a compound having the structure of M-III:

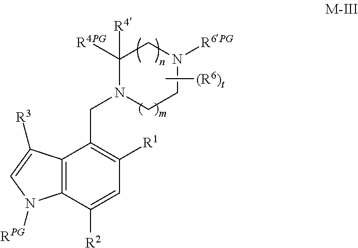

or a salt thereof to provide a compound of formula M-II or a salt thereof.
312. The method of Embodiment 294, wherein the compound of formula M-II or a salt thereof is a compound of any one of Embodiments 3-279.
313. The method of Embodiment 294 or 312, wherein the compound of formula M-III or a salt thereof is a compound of any one of Embodiments 4-279.
314. The method of any one of Embodiments 294-313, wherein R^{6′PG} is removed.
315. The method of any one of Embodiments 294-314, wherein R^{6′PG} is Bn.
316. The method of any one of Embodiments 282-310, comprising a method of any one of Embodiments 311-315.
317. A method comprising reacting a compound of formula M-IV:

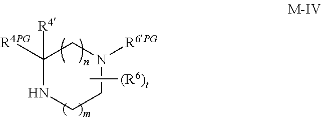

or a salt thereof, with a compound of formula M-V or a salt thereof:

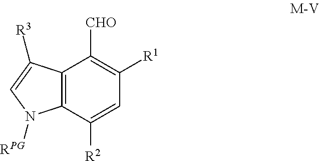

under a condition to provide a compound of formula M-III or a salt thereof.
318. The method of Embodiment 317, wherein the compound of formula M-III or a salt thereof is a compound of any one of Embodiments 4-279.
319. The method of Embodiment 317 or 318, wherein the compound of formula M-IV or a salt thereof is a compound of any one of Embodiments 24-279.
320. The method of any one of Embodiments 317-319, wherein the reaction is performed in the presence of a reducing agent.
321. The method of any one of Embodiments 282-316, comprising a method of any one of Embodiments 317-320.
322. A method comprising protecting a group and/or de-protecting a group of a compound of formula M-VI:

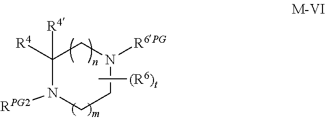

or a salt thereof to provide a compound of formula M-IV or a salt thereof.
323. The method of Embodiment 322, wherein the compound of formula M-IV or a salt thereof is a compound of any one of Embodiments 24-279.
324. The method of Embodiment 322 or 323, wherein the compound of formula M-VI or a salt thereof is a compound of any one of Embodiments 25-279.
325. The method of any one of Embodiments 322-324, wherein R^{PG2} is —Boc.
326. The method of any one of Embodiments 282-321, comprising a method of any one of Embodiments 322-325.
327. A method comprising converting a compound of formula M-VII:

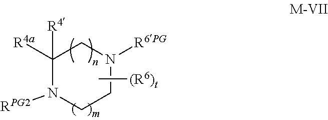

or a salt thereof to provide a compound of formula M-VI or a salt thereof.
328. The method of Embodiment 327, wherein the compound of formula M-VI or a salt thereof is a compound of any one of Embodiments 25-279.
329. The method of Embodiment 327 or 328, wherein the compound of formula M-VII or a salt thereof is a compound of any one of Embodiments 26-279.
330. The method of any one of Embodiments 327-329, comprising oxidizing a —CHO group in R^{4a} to a —COOH group.
331. The method of any one of Embodiments 282-326, comprising a method of any one of Embodiments 327-330.
332. A method comprising protecting a group in a compound of formula M-VIII:

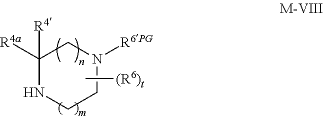

or a salt thereof to provide a compound of formula M-VII or a salt thereof.
333. The method of Embodiment 332, wherein the compound of formula M-VII or a salt thereof is a compound of any one of Embodiments 26-279.
334. The method of Embodiment 332 or 333, wherein the compound of formula M-VIII or a salt thereof is a compound of any one of Embodiments 27-279.
335. The method of any one of Embodiments 282-331, comprising a method of any one of Embodiments 332-334.
336. A method comprising converting a compound of formula M-IX:

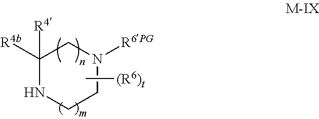

or a salt thereof to provide a compound of formula M-VIII or a salt thereof.
337. The method of Embodiment 336, wherein the compound of formula M-VIII or a salt thereof is a compound of any one of Embodiments 27-279.
338. The method of Embodiment 336 or 337, wherein the compound of formula M-IX or a salt thereof is a compound of any one of Embodiments 28-279.
339. The method of any one of Embodiments 336-338, comprising oxidizing a —CH₂OH group in R^{4b} to a —CHO group.
340. The method of any one of Embodiments 282-335, comprising a method of any one of Embodiments 336-339.
341. A method comprising reducing a compound of formula M-X:

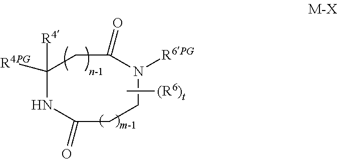

or a salt thereof to provide a compound of formula M-IX or a salt thereof.
342. The method of Embodiment 341, wherein the compound of formula M-IX or a salt thereof is a compound of any one of Embodiments 28-279.
343. The method of Embodiment 341 or 342, wherein the compound of formula M-X or a salt thereof is a compound of any one of Embodiments 29-279.
344. The method of any one of Embodiments 282-340, comprising a method of any one of Embodiments 341-343.
345. A method comprising converting a compound of formula M-XI:

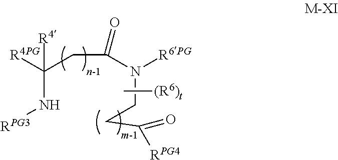

or a salt thereof to provide a compound of formula M-X or a salt thereof.
346. The method of Embodiment 345, wherein the compound of formula M-X or a salt thereof is a compound of any one of Embodiments 29-279.
347. The method of Embodiment 345 or 346, wherein the compound of formula M-XI or a salt thereof is a compound of any one of Embodiments 30-279.
348. The method of any one of Embodiments 282-344, comprising a method of any one of Embodiments 345-347.
349. A method comprising reacting a compound of formula M-XII:

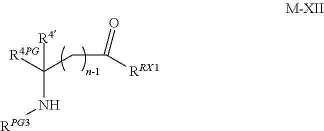

or a salt thereof, with a compound of formula M-XIII:

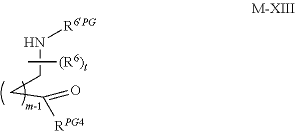

or a salt thereof to provide a compound of formula M-XI or a salt thereof.
350. The method of Embodiment 349, wherein the compound of formula M-XI or a salt thereof is a compound of any one of Embodiments 30-279.
351. The method of Embodiment 349 or 350, wherein the compound of formula M-XII or a salt thereof is a compound of any one of Embodiments 31-279.
352. The method of any one of Embodiments 349-351, wherein the compound of formula M-XIII or a salt thereof is a compound of any one of Embodiments 32-279.
353. The method of any one of Embodiments 282-348, comprising a method of any one of Embodiments 349-352.
354. A method comprising converting a compound of formula M-XIV:

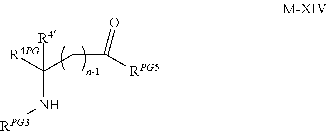

or a salt thereof to a compound of formula M-XII or a salt thereof.
355. The method of Embodiment 354, wherein the compound of formula M-XII or a salt thereof is a compound of any one of Embodiments 31-279.
356. The method of Embodiment 354 or 355, wherein the compound of formula M-XIV or a salt thereof is a compound of any one of Embodiments 33-279.
357. The method of any one of Embodiments 282-353, comprising a method of any one of Embodiments 354-356.
358. A method comprising reacting a compound of formula M-IV′:

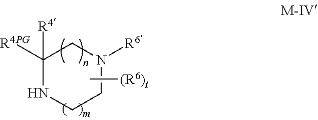

or a salt thereof, with a compound of formula M-V or a salt thereof:

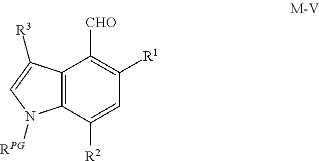

under a condition to provide a compound of formula M-I or a salt thereof.
359. The method of Embodiment 358, wherein the compound of formula M-I or a salt thereof is a compound of any one of Embodiments 2-238.
360. The method of Embodiment 358 or 359, wherein the compound of formula M-IV′ or a salt thereof is a compound of any one of Embodiments 34-279.
361. The method of any one of Embodiments 358-360, wherein the reaction is performed in the presence of a reducing agent.
362. The method of any one of Embodiments 282-357, comprising a method of any one of Embodiments 358-361.
363. A method comprising de-protecting a group of a compound of formula M-XV:

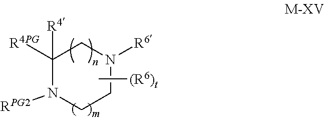

or a salt thereof to provide a compound of formula M-IV′ or a salt thereof.
364. The method of Embodiment 363, wherein the compound of formula M-IV′ or a salt thereof is a compound of any one of Embodiments 34-279.
365. The method of Embodiment 363 or 364, wherein the compound of formula M-XV or a salt thereof is a compound of any one of Embodiments 35-279.
366. The method of any one of Embodiments 282-362, comprising a method of any one of Embodiments 363-365.
367. A method comprising converting compound of formula M-XVI:

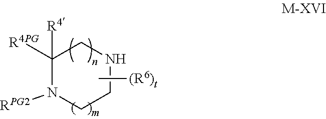

or a salt thereof to a compound of formula M-XV or a salt thereof.
368. The method of Embodiment 367, wherein the compound of formula M-XV or a salt thereof is a compound of any one of Embodiments 35-279.
369. The method of Embodiment 367 or 368, wherein the compound of formula M-XVI or a salt thereof is a compound of any one of Embodiments 36-279.
370. The method of any one of Embodiments 367-369, wherein the method is or comprises a reductive amination reaction with an aldehyde or ketone whose structure is such that when its —C(O)— is replaced with —CH₂—, a compound having the structure of R^{6′}—H or a salt thereof is formed.
371. The method of any one of Embodiments 367-369, wherein the method is or comprises an amination reaction.
372. The method of any one of Embodiments 367-369, wherein the method is or comprises an amination reaction with R^{6′}-boronic acid.
373. The method of any one of Embodiments 367-369, wherein the method is or comprises an acylating reaction, wherein an acylating agent has the structure of R^{6x1}—C(O)—C₁-C₆ aliphatic or a salt thereof.
374. The method of any one of Embodiments 367-369, wherein the method is or comprises an sulfonylation reaction, wherein a sulfonylation agent has the structure of R^{6x2}—S(O)₂—C₁-C₆ aliphatic or a salt thereof.
375. The method of any one of Embodiments 282-366, comprising a method of any one of Embodiments 367-374.
376. A method comprising de-protecting a group of a compound of formula M-XVII:

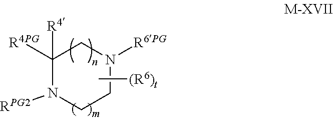

or a salt thereof to a compound of formula M-XVI or a salt thereof.
377. The method of Embodiment 376, wherein the compound of formula M-XVI or a salt thereof is a compound of any one of Embodiments 36-279.
378. The method of Embodiment 376 or 377, wherein the compound of formula M-XVII or a salt thereof is a compound of any one of Embodiments 37-279.
379. The method of any one of Embodiments 282-375, comprising a method of any one of Embodiments 376-378.
380. A method comprising protecting a group of a compound of formula M-IV or a salt thereof to provide a compound of formula M-XVII or a salt thereof.
381. The method of Embodiment 380, wherein the compound of formula M-XVII or a salt thereof is a compound of any one of Embodiments 37-279.
382. The method of Embodiment 380 or 381, wherein the compound of formula M-IV or a salt thereof is a compound of any one of Embodiments 24-279.
383. The method of any one of Embodiments 282-379, comprising a method of any one of Embodiments 380-382.
384. A method for assessing a compound, comprising utilizing an assay described in the Examples.
385. A method of inhibiting a C3 convertase, comprising contacting a C3 convertase with a compound or composition of any one of the preceding Embodiments.
386. A method of modulating complement alternative pathway activity in a subject, wherein the method comprises administering to the subject a therapeutically effective amount of a compound or composition of any one of the preceding Embodiments.
387. A method of treating a disorder or a disease in a subject mediated by complement activation, wherein the method comprises administering to the subject a therapeutically effective amount of a compound or composition of any one of the preceding Embodiments.
388. A method of treating a disorder or a disease in a subject mediated by activation of the complement alternative pathway, wherein the method comprises administering to the subject a therapeutically effective amount of a compound or composition of any one of the preceding Embodiments.
389. The method of any one of Embodiments 385-388, wherein the subject is suffering from a disease or disorder selected from age-related macular degeneration, geographic atrophy, Stargardt's disease, diabetic retinopathy, uveitis, glaucoma, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Creutzfeld-Jacob disease, Guillain Barre Syndrome, spinal cord injury, traumatic brain injury, Alzheimer's disease, Parkinson's disease, progressive supranuclear palsy, corticobasal syndrome, Pick's disease, mild cognitive impairment, Huntington's disease, diabetic neuropathy, neuropathic pain syndromes, fibromyalgia, frontotemporal dementia, dementia with Lewy bodies, multiple system atrophy, leptomeningeal metastasis, amyotrophic lateral sclerosis (ALS), chronic inflammatory demyelinating polyneuropathy (CIDP), neuromyelitis optica (NMO), disorders of inappropriate or undesirable complement activation, hemodialysis complications, graft rejection (e.g., hyperacute allograft rejection, xenograft rejection, interleukin-2 induced toxicity during IL-2 therapy, inflammatory disorders, inflammation of autoimmune diseases, Crohn's disease, acute respiratory distress syndrome (ARDS), myocarditis, postischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, asthma, chronic obstructive pulmonary disease (COPD), emphysema, pulmonary embolisms and infarcts, pneumonia, fibrogenic dust diseases, pulmonary fibrosis, rhinosinusitis, nasal polyposis, allergy, bronchoconstriction, hypersensitivity pneumonitis, parasitic diseases, Goodpasture's Syndrome, pulmonary vasculitis, pauci-immune vasculitis, thrombotic microangiopathy (TMA), immune complex-associated inflammation, antiphospholipid syndrome, glomerulonephritis, C3 glomerulopathy, IgA nephropathy, cancer, periodontitis, gingivitis, and obesity.
390. The method of Embodiment 389, wherein the condition, disorder or disease is hemolytic anemia, and the subject is suffering from paroxysmal nocturnal hemoglobinuria.
391. The method of Embodiment 389, wherein the condition, disorder or disease is hemolytic anemia, and the subject is suffering from autoimmune hemolytic anemia (e.g., cold agglutinin disease or warm autoimmune hemolytic anemia).
392. The method of any one of Embodiments 385-388, wherein the subject suffers from TMA secondary to atypical hemolytic uremic syndrome.
393. The method of any one of Embodiments 385-388, wherein the subject suffers from TMA secondary to hematopoietic stem cell transplant (HSCT-TMA).
394. The method of any one of Embodiments 385-388, wherein the subject suffers from drug-induced TMA.
395. The method of any one of Embodiments 385-388, wherein the subject suffers from complement activation secondary to administration of another therapeutic or diagnostic agent.
396. The method of any one of Embodiments 385-388, wherein the subject suffers from complement-mediated disorder is complement activation secondary to gene therapy (e.g., gene therapy with a viral vector such as an adeno-associated virus (AAV), adenovirus, or lentivirus vector).
397. The method of any one of Embodiments 385-388, wherein the subject suffers from complement-mediated disorder is complement activation secondary to cell therapy.
398. A method of treating age related macular degeneration, comprising administering to a subject suffering therefrom a therapeutically effective amount of a compound or composition of any one of the preceding Embodiments.
399. The method of Embodiment 398, wherein the age related macular degeneration is intermediate age-related macular degeneration.
400. The method of any one of Embodiments 385-399, wherein another therapeutic agent is administered such that a subject is exposed to the effects of both the compound and the another therapeutic agent.
401. The method of any one of Embodiments 385-400, wherein another therapeutic agent is administered prior to, concurrently with or subsequently to the administration of the compound.

### EXEMPLIFICATION

Technologies of the present disclosure may be understood more readily by reference to the following detailed description of certain embodiments.

### Example 1: Preparation of Compounds—Certain Useful Procedurals

Those skilled in the art appreciate that various technologies are available for preparing provided compounds and compositions in accordance with the present disclosure. For example, in some embodiments, technologies described below were utilized to prepare certain compounds.

### General Experimental Procedure 1: Reductive Amination

To a solution of tert-butyl 5-methoxy-4-((2-(4-(methoxycarbonyl)phenyl)piperazin-1-yl)methyl)-7-methyl-1H-indole-1-carboxylate (1 eq) in DCM (0.5 mL) was added aldehyde or ketone (4 eq) followed by 5 M acetic acid in DCM (1 eq). After 15 min sodium triacetoxyborohydride (4 eq) was added and the mixture stirred at 20° C. for 15 h. The reaction was worked-up; concentrated under reduced pressure and the residue used as is in the subsequent step.

### General Experimental Procedure 2: Boc Deprotection and Ester Hydrolysis

The crude from General Experimental Procedure 1 (1 eq) was dissolved in MeOH/H₂O (5:1, 1.2 mL), 50 wt. % aq. NaOH (20 eq) was added, and the mixture stirred at 45° C. for 15 h. The reaction mixture was neutralized to pH 7 with the addition of 37 wt. % aq. HCl and purified by automated reverse phase FCC to afford the desired product.

### General Experimental Procedure 3: Piperazine Alkylation

In some embodiments, the present disclosure provides technologies for piperazine alkylation. In some embodiments, a provided technology comprising reacting a piperazine, e.g., a compound of formula M-II or a salt thereof, a compound of formula I wherein R^{6′} is —H or a salt thereof, with an electrophile, an alkylating reagent, or a compound having the structure of R^{6x3}—R^{6′} or a salt thereof, wherein R^{6x3} is a leaving group. In some embodiments, a reaction is performed as the following: to a solution of tert-butyl (R)-5-methoxy-4-((2-(4-(methoxycarbonyl)phenyl)piperazin-1-yl)methyl)-7-methyl-1H-indole-1-carboxylate (1 eq.) and Et₃N (1.8 Eq.) in THE (10 mM) was added alkylating reagent (1.5 Eq.). The resulting solution was stirred at 20° C. for 15 h. The reaction was worked-up; concentrated under reduced pressure and purified by normal phase FCC to afford the desired product.

### General Experimental Procedure 4: N-acylsulfonamide Synthesis

In some embodiments, the present disclosure provides technologies for preparing N-acylsulfonamide, e.g., a compound of formula I wherein R⁴ is substituted with —C(O)NHSO₂C₁-C₄alkyl or a salt thereof. In some embodiments, a provided technology comprises reacting a carboxylic acid or a salt thereof, e.g., a compound of formula I wherein R⁴ is substituted with —COOH or a salt thereof, with a sulfonamide, e.g., a compound having the structure of NH₂SO₂C₁-C₄alkyl or a salt thereof, in the presence of a condensation reagent (e.g., in some embodiments, in the presence of a base/acyl transfer reagent, e.g., DMAP and/or a carboxyl activating agent, e.g., a carbodiimide such as a salt of EDC (e.g., EDC HCl)). In some embodiments, a reaction is performed as the following: to a solution of carboxylic acid (1.0 eq.) in DMF (50 mM) were added sulfonamide (2.0 eq.), DMAP (2.0 eq.) and EDC HCl (2.0 eq.). The resulting mixture was stirred at 20° C. for 3 h. The crude reaction mixture was directly purified by automated reverse phase FCC to afford the desired product.

LCMS: In some embodiments, LCMS was utilized to purify or characterize provided compounds or compositions.

In some embodiments, an instrument employed was the Agilent 1290 series with UV detector and HP 6130 MSD mass detector equipped with a Waters XBridge BEH C18 XP (2.1×50 mm, 2.5 m) column. Those skilled in the art appreciate that other systems/methods may also be utilized in accordance with the present disclosure. Certain methods are described below as examples:

### LCMS Method “31697 LCMS-5 C3”

Mobile phase A: Ammonium acetate (10 mM) in water/methanol/acetonitrile (900:60:40)
Mobile phase B: Ammonium acetate (10 mM) in water/methanol/acetonitrile (100:540:360)
Pump Flow: 0.6 ml/min
UV Detection: 215 nm
Injection Volume: 0.1 μL
Run Time: 3.5 min
Column Temperature: 35° C.
Pump Program: Gradient

| Time (min) | % A | % B |
|---|---|---|
| 0.0 | 95 | 5 |
| 0.5 | 95 | 5 |
| 2.5 | 0 | 100 |
| 3.5 | 0 | 100 |

### LCMS Method “31697S TFA LCMS-5 C1”

Column: Water Acquity UPLC HHS T3 (2.1×75 mm; 1.8 μm)
Mobile phase A: 0.05% TFA in Milli-Q
Mobile phase B: Acetonitrile
Pump Flow: 0.6 ml/min
UV Detection: 220 nm
Injection Volume: 0.2 μL
Run Time: 3.5 min
Column Temperature: 35° C.
Pump Program: Gradient

| Time (min) | % A | % B |
|---|---|---|
| 0.0 | 95 | 5 |
| 0.5 | 95 | 5 |
| 2.5 | 10 | 90 |
| 3.5 | 10 | 90 |

### LCMS Method “31697B TFA LCMS-5 C3”

Mobile phase A: 0.05% TFA in Milli-Q
Mobile phase B: Acetonitrile
Pump Flow: 0.6 ml/min
UV Detection: 220 nm
Injection Volume: 0.5 μL
Run Time: 3.0 min
Column Temperature: 35° C.
Pump Program: Gradient

| Time (min) | % A | % B |
|---|---|---|
| 0.0 | 95 | 5 |
| 0.5 | 95 | 5 |
| 2.5 | 10 | 90 |
| 3.5 | 10 | 90 |

In some embodiments, an instrument employed was the Water Acquity H-Class with PDA detector and SQD mass detector equipped with a Waters XBridge BEH C18 XP (2.1×50 mm, 2.5 m) column. The mass detector employed the atmosphere pressure ionization via electrospray for both positive and negative conjugates.

### LCMS Method “General 3”

Mobile phase A: 10 mM ammonium bicarbonate in water (pH 9.5)
Mobile phase B: Acetonitrile
Pump Flow: 0.6 ml/min
UV Detection: λmax
Injection Volume: 1.0 μL
Run Time: 3.0 min
Column Temperature: 30° C.
Pump Program: Gradient

| Time (min) | % A | % B |
|---|---|---|
| 0.0 | 80 | 20 |
| 1.5 | 5 | 95 |
| 2.5 | 5 | 95 |
| 2.6 | 80 | 20 |

### LCMS Method “General 4”

Mobile phase A: 10 mM ammonium bicarbonate in water (pH 9.5)
Mobile phase B: Acetonitrile
Pump Flow: 0.6 ml/min
UV Detection: λmax
Injection Volume: 1.0 μL
Run Time: 3.0 min
Column Temperature: 30° C.
Pump Program: Gradient

| Time (min) | % A | % B |
|---|---|---|
| 0.0 | 95 | 5 |
| 1.5 | 5 | 95 |
| 2.5 | 5 | 95 |
| 2.6 | 95 | 5 |

### LCMS Method “General 3 Acidic”

Mobile phase A: 0.1% TFA in Milli-Q
Mobile phase B: Acetonitrile
Pump Flow: 0.6 ml/min
UV Detection: λmax
Injection Volume: 0.4 μL
Run Time: 3.0 min
Column Temperature: 30° C.
Pump Program: Gradient

| Time (min) | % A | % B |
|---|---|---|
| 0.0 | 80 | 20 |
| 1.5 | 5 | 95 |
| 2.5 | 5 | 95 |
| 2.6 | 85 | 15 |

In some embodiments, an instrument employed was the Agilent 1200 series with ELSD 1290 detector, UV detector and Agilent 6130 mass detector equipped with a Waters XBridge BEH XP (2.1×50 mm, 2.5 m) column. The mass detector employed the atmosphere pressure ionization via electrospray for both positive and negative conjugates.

### LCMS Method “General3 LCMS-4 AB”

Mobile phase A: Ammonium acetate (10 mM) in water/methanol/acetonitrile (900:60:40)
Mobile phase B: Ammonium acetate (10 mM) in water/methanol/acetonitrile (100:540:360)
Pump Flow: 0.6 ml/min
UV Detection: 215, 254 nm
Injection Volume: 0.3 μL
Run Time: 3.5 min
Column Temperature: 35° C.
Pump Program: Gradient

| Time (min) | % A | % B |
|---|---|---|
| 0.0 | 80 | 20 |
| 1.5 | 0 | 100 |
| 3.5 | 0 | 100 |

In some embodiments, a useful preparative HPLC protocol is as described below (in some embodiments, the instrument employed was Agilent 1200 series with UV detector):

### LCMS Method “31697-2101B-prep”

Column: Chiralpak IC (250×20 mm, 5 μm)
Mobile phase A: Heptane/isopropanol (70/30)
Pump Flow: 14 ml/min
UV Detection: 215 nm
Sample concentration: 41 mg/mL in methanol (+ few drops of THF)
Injection Volume: 100 μL
Run Time: 17.0 min
Column Temperature: Ambient
Pump Program: Isocratic

| Time (min) | % A | % B |
|---|---|---|
| Initial | 95 | 5 |
| 2 | 95 | 5 |
| 12 | 70 | 30 |
| 14 | 70 | 30 |
| 15 | 95 | 5 |

In some embodiments, SFC was utilized. In some embodiments, preparative SFC was utilized. In some embodiments, an instrument employed was a Water SFC prep system with PDA detector. Certain methods are described below as examples:

### SFC

### Preparative SFC

The instrument employed was the Water SFC prep system with PDA detector.

SFC Method “31697 EKN IG 5-30% 18 min”
Column: Chiralpak IG (250×20 mm, 5 μm, PN:87445)
Mobile phase A: CO₂
Mobile phase B: 0.2% ammonia in methanol
Pump Flow: 70 ml/min
PDA Detection: 215 nm
Sample concentration: 21 mg/mL in methanol
Injection Volume: 200 μL
Run Time: 18.0 min
Column Temperature: 40° C.
Pump Program: Gradient

| Time (min) | % A | % B |
|---|---|---|
| Initial | 95 | 5 |
| 2 | 95 | 5 |
| 12 | 70 | 30 |
| 14 | 70 | 30 |
| 15 | 95 | 5 |

### SFC Method “31697-1051-04”

Column: Chiralpak IG (250×20 mm; 5.0 μm)
Mobile phase A: CO₂
Mobile phase B: 0.2% ammonia in methanol
Pump Flow: 70 ml/min
PDA Detection: 215 nm
Sample concentration: 12.5 mg/mL in methanol
Injection Volume: 1500 μL
Run Time: 7.0 min
Column Temperature: 40° C.
Pump Program: Gradient

| Time (min) | % A | % B |
|---|---|---|
| 0.0 | 80 | 20 |
| 1.0 | 80 | 20 |
| 4.0 | 60 | 40 |
| 6.0 | 60 | 40 |
| 6.5 | 96 | 4 |
| 7.0 | 96 | 4 |

SFC Method “31697EKN ADH4 40% 20 min”
Column: Chiralpak AD-H (250×20 mm; 5.0 μm)
Mobile phase A: CO₂
Mobile phase B: 0.2% ammonia in ethanol
Pump Flow: 70 ml/min
PDA Detection: 215 nm
Sample concentration: ˜100 mg/mL in methanol
Injection Volume: 1000 μL
Run Time: 15 min
Column Temperature: 40° C.
Pump Program: Gradient

| Time (min) | % A | % B |
|---|---|---|
| 0.0 | 96 | 4 |
| 6.0 | 60 | 40 |
| 15.0 | 60 | 40 |

In some embodiments, analytical SFC was utilized. In some embodiments, an instrument employed was the Water Acquity UPC² with PDA detector and QDA mass detector. The mass detector employed the electrospray for both positive and negative conjugates (100-1250 Da).

SFC Method “IG grad MA1”
Column: Diacel Chiralpak (3.0×150 mm; 3.0 m)
Mobile phase A: CO₂
Mobile phase B: 0.2% ammonia in methanol
Pump Flow: 1.0 ml/min
UV Detection: 254 nm
Injection Volume: 2.0 μL
Run Time: 10.0 min
Column Temperature: 40° C.
ABPR: 2000 psi
Pump Program: Gradient

| Time (min) | % A | % B | Curve |
|---|---|---|---|
| Initial | 98 | 2 | Initial |
| 6.0 | 60 | 40 | 6 |
| 9.0 | 60 | 40 | 6 |
| 9.1 | 98 | 2 | 6 |

### SFC Method “31697F UPC2 10 m Cel2 MA1”

Column: Waters UPC² Trefoil CEL2 (3.0×150 mm, 2.5 m)
Mobile phase A: CO₂
Mobile phase B: 0.2% NH₄OH (25 wt. % aq.) in methanol
Pump Flow: 2.5 ml/min
UV Detection: 220 nm
Injection Volume: 1.0 μL
Run Time: 10.0 min
Column Temperature: 40° C.
ABPR: 2000 psi
Pump Program: Gradient

| Time (min) | % A | % B | Curve |
|---|---|---|---|
| 0.0 | 98 | 2 | Initial |
| 6.0 | 60 | 40 | 5 |
| 9.0 | 60 | 40 | 6 |
| 9.1 | 98 | 2 | 6 |

SFC Method “Amy grad EE”
Column: Aquity UPC² Trefoil AMY (3.0×150 mm, 2.5 m)
Mobile phase A: CO₂
Mobile phase B: 0.2% Et₂NH in Ethanol
Pump Flow: 2.5 ml/min
UV Detection: 254 nm
Injection Volume: 1.0 μL
Run Time: 10.0 min
Column Temperature: 40° C.
ABPR: 2000 psi
Pump Program: Gradient

| Time (min) | % A | % B | Curve |
|---|---|---|---|
| 0.0 | 98 | 2 | Initial |
| 6.0 | 60 | 40 | 6 |
| 9.0 | 60 | 40 | 6 |
| 9.1 | 98 | 2 | 6 |

### SFC Method: “31697Q UPC² 4m Cel4”

Column: Phenomenex (3.0×150 mm; 3 μm)
Mobile phase A: CO₂
Mobile phase B: Methanol
Pump Flow: 2.5 ml/min
UV Detection: 234 nm
Injection Volume: 1.0 μL
Run Time: 4 min
Column Temperature: 40° C.
ABPR: 2000 psi
Pump Program: Gradient

| Time (min) | % A | % B | Curve |
|---|---|---|---|
| Initial | 98 | 2 | Initial |
| 3.0 | 85 | 15 | 6 |
| 3.1 | 98 | 2 | 11 |

SFC Method: “IC3 grad MA1”
Column: Daicel Chiralpak IC-3 (3.0×150 mm; 3 μm)
Mobile phase A: CO₂
Mobile phase B: 0.2% NH₄OH (25 wt. % aq.) in methanol
Pump Flow: 1.2 ml/min
UV Detection: 254 nm
Injection Volume: 1.0 μL
Run Time: 10 min
Column Temperature: 40° C.
ABPR: 200 psi
Pump Program: Gradient

| Time (min) | % A | % B | Curve |
|---|---|---|---|
| Initial | 98 | 2 | Initial |
| 6.0 | 60 | 40 | 6 |
| 9.0 | 60 | 40 | 6 |
| 9.1 | 98 | 2 | 6 |

### Example 2: tert-Butyl 5-methoxy-4-((2-(4-(methoxycarbonyl)phenyl)piperazin-1-yl)methyl)-7-methyl-1H-indole-1-carboxylate

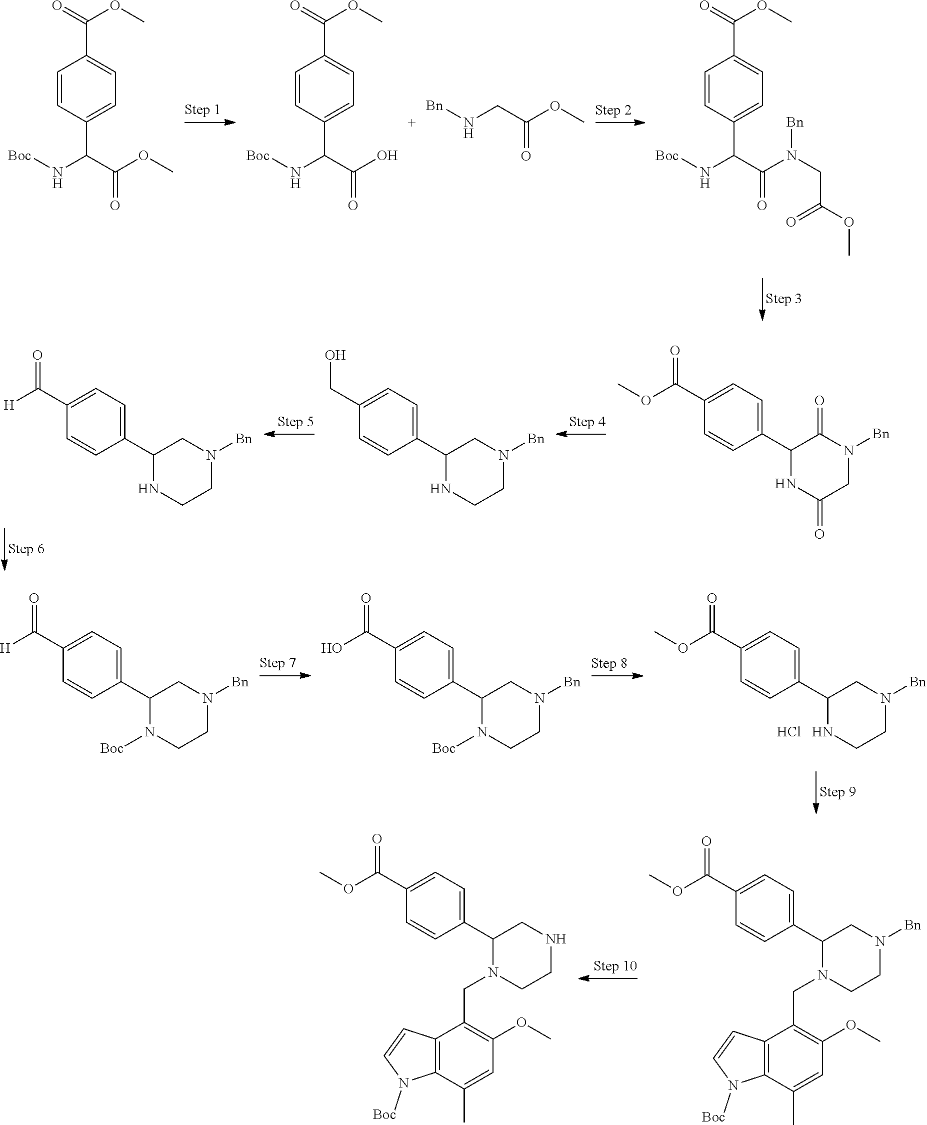

Step 1. 2-((tert-Butoxycarbonyl)amino)-2-(4-(methoxycarbonyl)phenyl)acetic acid was prepared with the following procedure. To a solution of methyl 4-(1-((tert-butoxycarbonyl)amino)-2-methoxy-2-oxoethyl)benzoate (30 g, 1.0 eq, 93 mmol) in THF (170 mL) at 0° C. was dosed 1 M aq. LiOH (95 mL, 1.0 eq, 95 mmol) with a syringe pump (0.5 mL/min). The reaction was stirred at 0° C. overnight. The reaction was worked up; diluted with water (250 mL) and extracted with TBME (1×150 mL, 2×100 mL). The aqueous layer was acidified to pH 3 with 0.5 M aq. citric acid and extracted with TBME (1×150 mL, 2×100 mL). The pooled organic layers were washed with 0.5 M aq. citric acid (2×50 mL), water, brine, dried over Na₂SO₄, filtered, and concentrated under reduced pressure to afford 2-((tert-butoxycarbonyl)amino)-2-(4-(methoxycarbonyl)phenyl)acetic acid (26.1 g, 91%) as an off-white solid. LCMS (General3 LCMS-4 AB) RT: 1.39 min; area % (254 nm): 100%; m/z=332.2 [M+Na]⁺.

Step 2. Methyl 4-(5-benzyl-11,11-dimethyl-3,6,9-trioxo-2,10-dioxa-5,8-diazadodecan-7-yl)benzoate was prepared with the following procedure. To a solution of 2-((tert-butoxycarbonyl)amino)-2-(4-(methoxycarbonyl)phenyl)acetic acid (26.6 g, 1.0 eq, 86.0 mmol) and methyl benzylglycinate (18.5 g, 1.2 eq, 103 mmol) under N₂ in DCM (350 mL) at 0° C. were added DIPEA (18.0 mL, 1.2 eq, 103 mmol), EDCI·HCl (19.8 g, 1.2 eq, 103 mmol), and HOBt (13.9 g, 1.2 eq, 103 mmol). The reaction mixture was warmed to 20° C. and stirred for 15 h. The reaction was worked-up; diluted with H₂O (350 ml) and the layers separated. The aqueous layer was extracted with DCM. The combined organic layers were washed with 1 M aq. HCl (200 mL), sat. aq. NaHCO₃ (200 mL), H₂O (200 mL), brine, dried over Na₂SO₄, and concentrated under reduced pressure to afford crude methyl 4-(5-benzyl-11,11-dimethyl-3,6,9-trioxo-2,10-dioxa-5,8-diazadodecan-7-yl)benzoate (36.2 g) as an orange sticky foam. LCMS (General 4) RT: 1.46 min; area % (254 nm): 76%; m/z=371.1 [(M-Boc)+H]⁺.

Step 3. Methyl 4-(4-benzyl-3,6-dioxopiperazin-2-yl)benzoate was prepared with the following procedure. To a solution of crude methyl 4-(5-benzyl-11,11-dimethyl-3,6,9-trioxo-2,10-dioxa-5,8-diazadodecan-7-yl)benzoate (36.2 g, 1.0 eq, 76.9 mmol) in DCM (110 mL) was added TFA (80 mL, 14 eq, 1.0 mol) and the mixture stirred at 20° C. for 15 h. Concentrated under reduced pressure and co-evaporated with toluene to remove residual TFA. The residue was dissolved in EtOAc (350 mL), sat. aq. NaHCO₃ (500 mL) was added and the mixture vigorously stirred at 20° C. for 15 h. The reaction was worked-up; the layers were separated and the aqueous layer extracted with EtOAc. The combined organic layers were washed with H₂O, brine, dried over Na₂SO₄, and concentrated under reduced pressure. The solid residue was triturated with TBME at 20° C. for 2 h. The solid was collected by filtration, washed with TBME, and dried under reduced pressure to afford methyl 4-(4-benzyl-3,6-dioxopiperazin-2-yl)benzoate (15.8 g, 61%) as a pale yellow solid. LCMS (General 3) RT: 0.83 min; area % (254 nm): 100%; m/z=337.2 [(M−H]⁻.

Step 4. (4-(4-Benzylpiperazin-2-yl)phenyl)methanol was prepared with the following procedure. To a suspension of LiAlH₄ (7.2 g, 4.1 eq, 0.19 mol) in dry THE (300 mL) under N₂ was added a solution of methyl 4-(4-benzyl-3,6-dioxopiperazin-2-yl)benzoate (15.8 g, 1.0 eq, 46.7 mmol) in dry THF (200 mL) dropwise over 1 h. The resulting suspension was stirred at 65° C. for 48 h. The reaction was worked-up; diluted with Et₂O (250 mL), cooled to 1° C. and quenched with the addition of water (7.5 mL), 15 wt. % aq. NaOH (7.5 mL), and more water (22.5 mL). The mixture was warmed to 20° C. and stirred for 2 h. Celite was added and the mixture stirred at 20° C. for 10 min before being filtered over Celite. The filter cake was washed with Et₂O and the filtrated concentrated under reduced pressure; upon standing the residue crystallized. The crystallized residue was triturated in n-pentane, the solid collected by filtration, washed with n-pentane, and dried under reduced pressure to afford (4-(4-benzylpiperazin-2-yl)phenyl)methanol (11.9 g, 91%) as a pale yellow solid. LCMS (General 4) RT: 0.96 min; area % (254 nm): 81%; m/z=283.2 [M+H]⁺.

Step 5. 4-(4-Benzylpiperazin-2-yl)benzaldehyde was prepared with the following procedure. To a solution of (4-(4-benzylpiperazin-2-yl)phenyl)methanol (11.9 g, 1.0 eq, 42.3 mmol) in DCM (400 mL) under N₂ was added manganese dioxide (41.77 g, 88 wt. %, 10.0 eq, 422.8 mmol). The resulting suspension was stirred at 20° C. for 15 h. The reaction was worked-up; filtered over Celite, the filter cake washed with DCM, and the filtrate concentrated under reduced pressure to afford crude 4-(4-benzylpiperazin-2-yl)benzaldehyde (10.5 g, 89%) was obtained as an orange oil. LCMS (General 4) RT: 1.13 min; area % (254 nm): 99%; m/z=281.3 [M+H]⁺.

Step 6. tert-Butyl 4-benzyl-2-(4-formylphenyl)piperazine-1-carboxylate was prepared with the following procedure. To a solution of 4-(4-benzylpiperazin-2-yl)benzaldehyde (10.5 g, 1.0 eq, 37.5 mmol) and DMAP (487 mg, 0.11 eq, 3.99 mmol) in DCM (350 mL) under N₂ at 0° C. was added Boc₂O (10.1 g, 1.24 eq, 46.3 mmol). The resulting mixture was warmed to 20° C. and stirred for 15 h. The reaction was worked-up; the reaction was concentrated under reduced pressure and the residue purified by automated FCC to afford tert-butyl 4-benzyl-2-(4-formylphenyl)piperazine-1-carboxylate (9.7 g, 68%) as a yellow oil. LCMS (General 4) RT: 1.72 min; area % (254 nm): 100%; m/z=481.4 [M+H]⁺.

Step 7. 4-(4-Benzyl-1-(tert-butoxycarbonyl)piperazin-2-yl)benzoic acid was prepared with the following procedure. To a solution of tert-butyl 4-benzyl-2-(4-formylphenyl)piperazine-1-carboxylate (3.93 g, 1.0 eq, 10.3 mmol) and 2-methyl-2-butene (90 wt. %, 80 mL, 66 eq, 0.68 mol) in t-BuOH (300 mL) was cooled on ice water (IT=5° C.). A solution of sodium dihydrogen phosphate (11.6 g, 9.4 eq, 96.7 mmol) and sodium chlorite (80 wt. %, 11.8 g, 10.1 eq, 104 mmol) in H₂O (100 mL) was dropwise added over 20 min. The reaction was warmed to 20° C. and stirred for 15 h. The reaction was worked-up; cooled on ice and quenched carefully with the addition of solid Na₂SO₃ (29 g, 230 mmol, 2.2 equiv. compared to NaO₂Cl). The reaction was stirred on ice for 4 h. The mixture was concentrated under reduced pressure and to the residue was added EtOAc. The layers were separated and the aqueous layer extracted with EtOAc (twice). The combined organic layers were washed with water, brine, dried over Na₂SO₄, and concentrated under reduced pressure to afford crude 4-(4-benzyl-1-(tert-butoxycarbonyl)piperazin-2-yl)benzoic acid (3.4 g, 83%) as a yellow solid. LCMS (General 4) RT: 0.83 min; area % (254 nm): 75%; m/z=395.3 [M−H]⁻.

Step 8. Methyl 4-(4-benzylpiperazin-2-yl)benzoate hydrochloride was prepared with the following procedure. To a solution of 4-(4-benzyl-1-(tert-butoxycarbonyl)piperazin-2-yl)benzoic acid (5.1 g, 1.0 eq, 13 mmol) in MeOH (50 mL) was added 4 N HCl in dioxane (16 mL, 4 molar, 5.0 eq, 64 mmol) and the mixture heated at 75° C. for 15 h. The reaction was worked-up; concentrated under reduced pressure to ˜15 mL, diluted the residue with Et₂O (125 mL) and triturated at 20° C. for 1 h. The solid was allowed to settle and the liquid portion decanted. Et₂O/acetone (1:1, 100 mL) was added and the mixture triturated at 20° C. for 2 h. The solid was collected by filtration, washed with acetone, Et₂O, and dried under reduced pressure to afford methyl 4-(4-benzylpiperazin-2-yl)benzoate hydrochloride (3.63 g, 81%) as a pale brown solid. LCMS (General 4) RT: 1.10 min; area % (254 nm): 88%; m/z=311.3 [M+H]⁺.

Step 9. tert-Butyl 4-((4-benzyl-2-(4-(methoxycarbonyl)phenyl)piperazin-1-yl)methyl)-5-methoxy-7-methyl-1H-indole-1-carboxylate was prepared with the following procedure. To a suspension of methyl 4-(4-benzylpiperazin-2-yl)benzoate hydrochloride (2.49 g, 1.0 eq, 7.18 mmol) in THE (100 mL) under N₂ was added Et₃N (1.50 mL, 1.5 eq, 10.8 mmol) and the mixture stirred at 20° C. for 15 min. To the suspension were added tert-butyl 4-formyl-5-methoxy-7-methyl-1H-indole-1-carboxylate (3.14 g, 1.51 eq, 10.9 mmol) and titanium(IV) isopropoxide (4.4 mL, 2.0 eq, 15 mmol) and continued to stir at 20° C. for 2 h. Added sodium triacetoxyborohydride (6.15 g, 4.04 eq, 29.0 mmol) and stirred at 20° C. for 15 h. The reaction was worked-up; concentrated under reduced pressure and portioned the residue between EtOAc and sat. aq. NaHCO₃. The layers were separated and the aqueous layer extracted with EtOAc. The combined organic layers were dried Na₂SO₄ and concentrated under reduced pressure. The residue was purified by automated FCC to afford tert-butyl 4-((4-benzyl-2-(4-(methoxycarbonyl)phenyl)piperazin-1-yl)methyl)-5-methoxy-7-methyl-1H-indole-1-carboxylate (1.67 g, 40%) as an off-white solid. LCMS (General 3) RT: 2.18 min; area % (254 nm): 100%; m/z=584.5 [M+H]⁺.

Step 10. tert-Butyl 5-methoxy-4-((2-(4-(methoxycarbonyl)phenyl)piperazin-1-yl)methyl)-7-methyl-1H-indole-1-carboxylate was prepared with the following protocol. To a solution of tert-butyl 4-((4-benzyl-2-(4-(methoxycarbonyl)phenyl)piperazin-1-yl)methyl)-5-methoxy-7-methyl-1H-indole-1-carboxylate (1.49 g, 1.0 eq, 2.55 mmol) in MeOH/AcOH (9:1, 30 mL) was added 20% Pd(OH)2 on carbon (125 mg, 0.349 eq, 890 μmol) as a suspension in MeOH (3 mL). The resulting suspension was hydrogenated at 70 psi in a pressure reactor at 20° C. for 15 h. The reaction was worked-up; filtered over Celite, the filter cake was washed with MeOH, and the filtrate concentrated under reduced pressure. The residue was diluted with a little water and the pH adjusted to ˜7 with 30% aq. NaOH. The mixture was extracted with EtOAc (3×). The combined organic layers were washed with water, dried over Na₂SO₄, and concentrated under reduced pressure. The residue was purified by automated FCC to afford tert-butyl 5-methoxy-4-((2-(4-(methoxycarbonyl)phenyl)piperazin-1-yl)methyl)-7-methyl-1H-indole-1-carboxylate (453 mg, 36%) as a pale yellow oil. LCMS (General 3) RT: 1.66 min; area % (254 nm): 92%; m/z=494.4 [M+H]⁺.

### Example 3: 4-(4-Benzyl-1-((5-methoxy-7-methyl-1H-indol-4-yl)methyl)piperazin-2-yl)benzoic acid

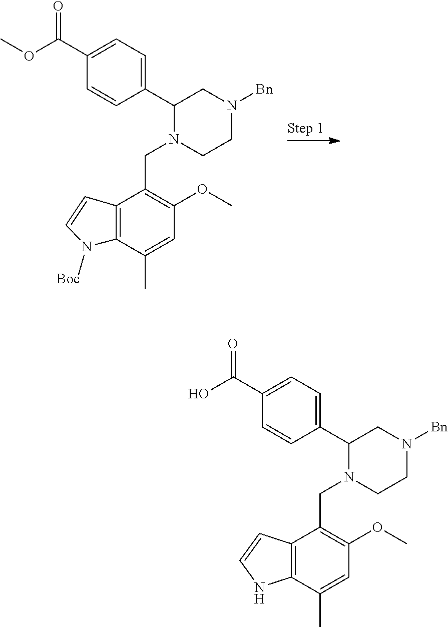

Step 1. 4-(4-Benzyl-1-((5-methoxy-7-methyl-1H-indol-4-yl)methyl)piperazin-2-yl)benzoic acid was prepared according to General Experimental Procedure 2. tert-Butyl 4-((4-benzyl-2-(4-(methoxycarbonyl)phenyl)piperazin-1-yl)methyl)-5-methoxy-7-methyl-1H-indole-1-carboxylate (65 mg, 111 μmol) gave 4-(4-benzyl-1-((5-methoxy-7-methyl-1H-indol-4-yl)methyl)piperazin-2-yl)benzoic acid (7.0 mg, 13%) as an off-white solid. LCMS (31697 LCMS-5 C3) RT: 2.104 min; area % (215 nm): 89.3%; m/z=470.2 [M+H]⁺. ¹H-NMR (299 MHz, Methanol-d₄) δ 8.09 (d, J=8.0 Hz, 2H), 7.62 (d, J=7.7 Hz, 2H), 7.42-7.13 (m, 6H), 6.71 (s, 1H), 6.34 (d, J=3.2 Hz, 1H), 4.10 (d, J=12.3 Hz, 1H), 3.87-3.76 (m, 1H), 3.74 (s, 3H), 3.67 (d, J=2.1 Hz, 2H), 3.19 (d, J=12.7 Hz, 1H), 2.97 (dd, J=19.9, 12.2 Hz, 3H), 2.70-2.52 (m, 1H), 2.52-2.39 (m, 4H), 1.95 (d, J=8.9 Hz, 1H).

### Example 4: 4-(1-((5-Methoxy-7-methyl-1H-indol-4-yl)methyl)-4-methylpiperazin-2-yl)benzoic acid

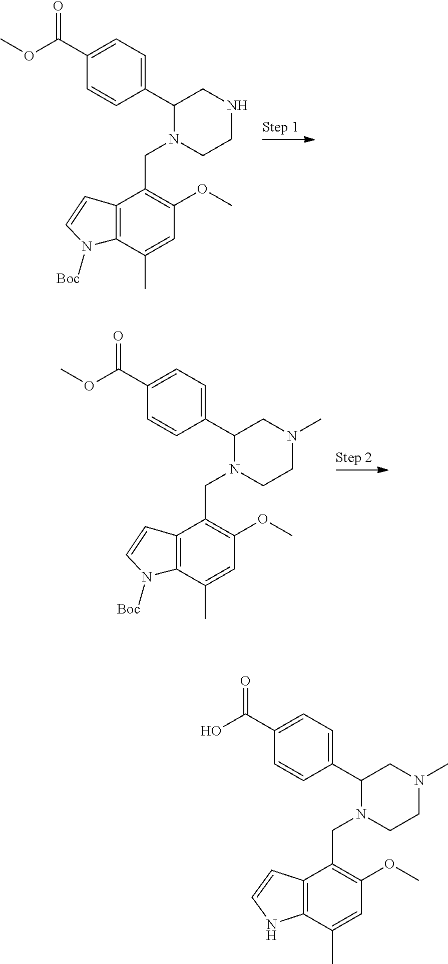

Step 1. tert-Butyl 5-methoxy-4-((2-(4-(methoxycarbonyl)phenyl)-4-methylpiperazin-1-yl)methyl)-7-methyl-1H-indole-1-carboxylate was prepared according to General Experimental Procedure 1. tert-Butyl 5-methoxy-4-((2-(4-(methoxycarbonyl)phenyl)piperazin-1-yl)methyl)-7-methyl-1H-indole-1-carboxylate (25 mg, 51 μmol) and 37 wt. % aq. formaldehyde (15 μL, 200 μmol) gave crude tert-butyl 5-methoxy-4-((2-(4-(methoxycarbonyl)phenyl)-4-methylpiperazin-1-yl)methyl)-7-methyl-1H-indole-1-carboxylate. LCMS (General 3) RT: 1.83 min; area % (254 nm): 95.9%; m/z=506.3 [M−H]⁻.

Step 2. 4-(1-((5-Methoxy-7-methyl-1H-indol-4-yl)methyl)-4-methylpiperazin-2-yl)benzoic acid was prepared according to General Experimental Procedure 2. Crude tert-butyl 5-methoxy-4-((2-(4-(methoxycarbonyl)phenyl)-4-methylpiperazin-1-yl)methyl)-7-methyl-1H-indole-1-carboxylate gave 4-(1-((5-methoxy-7-methyl-1H-indol-4-yl)methyl)-4-methylpiperazin-2-yl)benzoic acid (91.2 mg, 72% over two steps) as a white solid. LCMS (31697 LCMS-5 C3) RT: 1.375 min; area % (215 nm): 98.2%; m/z=394.2 [M+H]⁺. ¹H-NMR (299 MHz, DMSO-d₆) δ 10.83 (s, 1H), 7.99 (d, J=8.1 Hz, 2H), 7.69 (d, J=7.8 Hz, 2H), 7.26 (s, 1H), 6.66 (s, 1H), 6.43 (s, 1H), 3.71 (s, 3H), 3.57 (d, J=11.8 Hz, 1H), 3.52-3.39 (m, 2H), 3.24 (d, J=11.9 Hz, 1H), 2.90-2.57 (m, 3H), 2.42 (s, 3H), 2.36-1.84 (m, 5H).

### Example 5: 4-(1-((5-Methoxy-7-methyl-1H-indol-4-yl)methyl)-4-(methylsulfonyl)piperazin-2-yl)benzoic acid

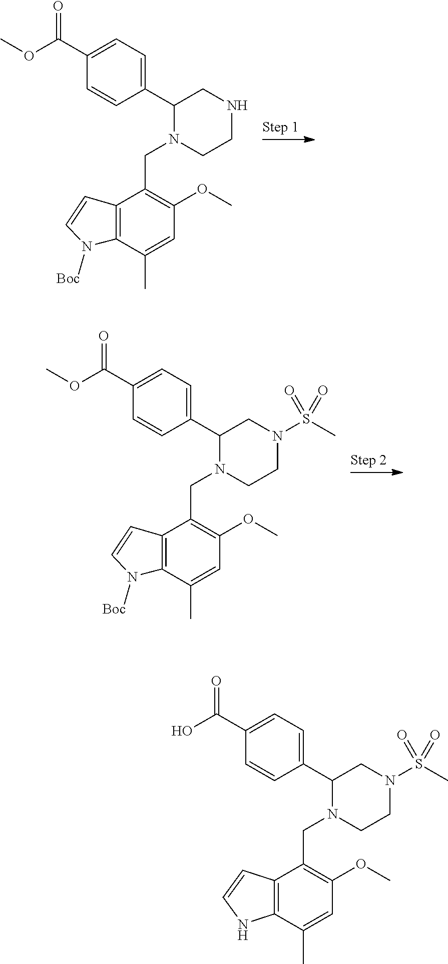

Step 1. tert-Butyl 5-methoxy-4-((2-(4-(methoxycarbonyl)phenyl)-4-(methylsulfonyl)piperazin-1-yl)methyl)-7-methyl-1H-indole-1-carboxylate was prepared with the following procedure. To a solution of tert-butyl 5-methoxy-4-((2-(4-(methoxycarbonyl)phenyl)piperazin-1-yl)methyl)-7-methyl-1H-indole-1-carboxylate (23 mg, 1.0 eq, 47 μmol) and Et₃N (9.4 mg, 13 μL, 2.0 eq, 93 μmol) in DCM (1.25 mL) under N₂ at 0° C. was added dropwise a 0.2 M solution of MsCl in DCM (250 μL, 1.0 eq, 47 μmol). The reaction was stirred at 0° C. for 15 min before being warmed to 20° C. and stirred for 15 h. The reaction was worked-up; washed with H₂O, dried over MgSO₄, and then purified over SCX-II (250 mg) to afford tert-butyl 5-methoxy-4-((2-(4-(methoxycarbonyl)phenyl)-4-(methylsulfonyl)piperazin-1-yl)methyl)-7-methyl-1H-indole-1-carboxylate (22 mg, 38 μmol, 82%) as a yellow oil. LCMS (General 3) RT: 1.74 min; area % (254 nm): 93%; m/z=570.1 [M−H]⁻.

Step 2. 4-(1-((5-Methoxy-7-methyl-1H-indol-4-yl)methyl)-4-(methylsulfonyl)piperazin-2-yl)benzoic acid was prepared with the following procedure. To a suspension of tert-butyl 5-methoxy-4-((2-(4-(methoxycarbonyl)phenyl)-4-(methylsulfonyl)piperazin-1-yl)methyl)-7-methyl-1H-indole-1-carboxylate (22 mg, 38 μmol) in MeOH/H₂O (4:1, 1.25 mL) was added 15 wt. % aq. NaOH (44 μL, 5.0 eq, 0.19 mmol) and the reaction mixture heated at 45° C. for 48 h. The reaction was worked-up; concentrated under reduced pressure and the residue was purified by automated reverse phase FCC to afford 4-(1-((5-methoxy-7-methyl-1H-indol-4-yl)methyl)-4-(methylsulfonyl)piperazin-2-yl)benzoic acid (6.7 mg, 38%) as a white solid. LCMS (31697 LCMS-5 C3) RT: 1.766 min; area % (215 nm): >99%; m/z=458.2 [M+H]⁺. ¹H-NMR (299 MHz, Methanol-d₄) δ 8.10 (d, J=8.4 Hz, 2H), 7.73 (d, J=8.0 Hz, 2H), 7.22 (d, J=3.1 Hz, 1H), 6.69 (s, 1H), 6.45 (d, J=3.1 Hz, 1H), 3.82 (d, J=11.9 Hz, 1H), 3.77 (s, 3H), 3.67-3.47 (m, 3H), 3.39 (d, J=11.8 Hz, 1H), 3.09-2.92 (m, 1H), 2.89 (d, J=11.8 Hz, 1H), 2.85-2.71 (m, 3H), 2.55-2.36 (m, 3H).

### Example 6: 4-(4-Acetyl-1-((5-methoxy-7-methyl-1H-indol-4-yl)methyl)piperazin-2-yl)benzoic acid

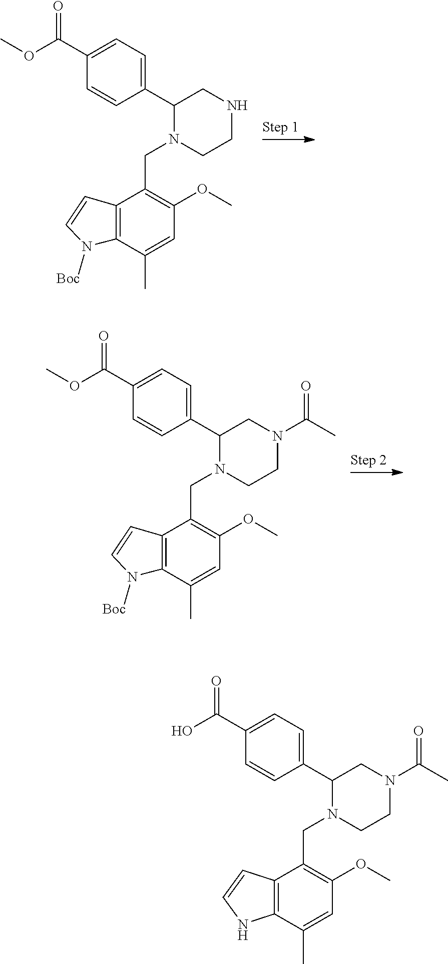

Step 1. tert-Butyl 4-((4-acetyl-2-(4-(methoxycarbonyl)phenyl)piperazin-1-yl)methyl)-5-methoxy-7-methyl-1H-indole-1-carboxylate was prepared with the following procedure. To a solution of tert-butyl 5-methoxy-4-((2-(4-(methoxycarbonyl)phenyl)piperazin-1-yl)methyl)-7-methyl-1H-indole-1-carboxylate (25 mg, 1.0 eq, 51 μmol) and Et₃N (10 mg, 14 μL, 2.0 eq, 0.10 mmol) in DCM (1.25 mL) under N₂ at 0° C. was added dropwise a solution of 0.2 M solution of acetyl chloride in DCM (250 μL, 1.0 eq, 51 μmol). The reaction was stirred at 0° C. for 15 min before being warmed to 20° C. and stirred for 15 h. The reaction was worked-up; washed with sat. aq. NaHCO₃, dried over Na₂SO₄, and concentrated under reduced pressure. Crude tert-butyl 4-((4-acetyl-2-(4-(methoxycarbonyl)phenyl)piperazin-1-yl)methyl)-5-methoxy-7-methyl-1H-indole-1-carboxylate was obtained as a yellow oil. LCMS (General 3) RT: 1.67 min; area % (254 nm): 98%; m/z=534.3 [M−H]⁻.

Step 2. 4-(4-Acetyl-1-((5-methoxy-7-methyl-1H-indol-4-yl)methyl)piperazin-2-yl)benzoic acid was prepared with the following procedure. To a suspension of crude tert-butyl 4-((4-acetyl-2-(4-(methoxycarbonyl)phenyl)piperazin-1-yl)methyl)-5-methoxy-7-methyl-1H-indole-1-carboxylate in MeOH/H₂O was added 15 wt. % aq. NaOH (60 μL, 5.0 eq, 0.25 mmol) and the mixture heated at 45° C. for 15 h. The reaction was worked-up; concentrated under reduced pressure and the residue was purified by automated reverse phase FCC to afford 4-(4-acetyl-1-((5-methoxy-7-methyl-1H-indol-4-yl)methyl)piperazin-2-yl)benzoic acid (7.2 mg, 33% over two steps) was obtained as a white solid. LCMS (31697 LCMS-5 C3) RT: 1.632 min; area % (215 nm): >99%; m/z=422.2 [M+H]⁺. ¹H-NMR (299 MHz, Methanol-d₄) δ 8.10 (dd, J=8.1, 3.0 Hz, 2H), 7.72 (t, J=7.9 Hz, 2H), 7.22 (d, J=3.1 Hz, 1H), 6.69 (s, 1H), 6.42 (dd, J=9.3, 3.1 Hz, 1H), 4.41 (d, J=12.3 Hz, 1H), 3.85 (d, J=12.1 Hz, 1H), 3.77 (s, 4H), 3.56 (dd, J=10.8, 3.0 Hz, 1H), 3.39 (dt, J=16.5, 4.0 Hz, 1H), 3.29-3.17 (m, 1H), 3.07-2.87 (m, 1H), 2.79 (t, J=12.3 Hz, 1H), 2.46 (s, 2H), 2.41-2.25 (m, 1H), 2.09 (d, J=3.1 Hz, 3H).

### Example 7: 4-(1-((5-Methoxy-7-methyl-1H-indol-4-yl)methyl)-4-phenylpiperazin-2-yl)benzoic acid

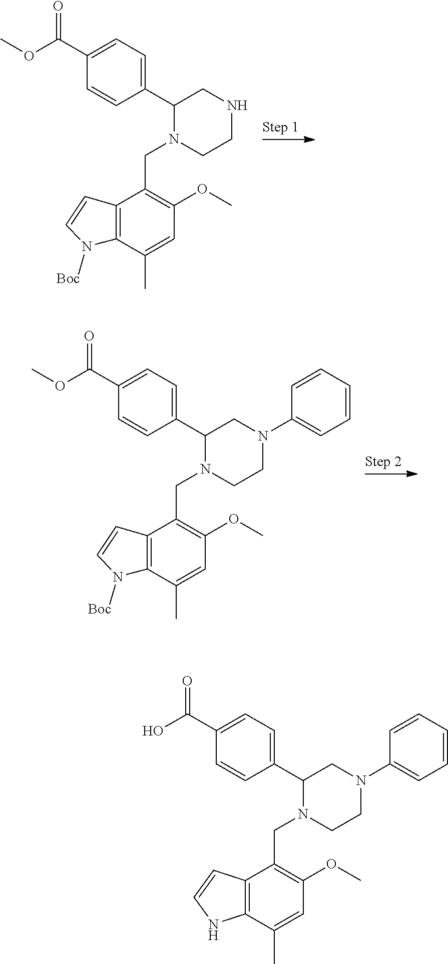

Step 1. tert-Butyl 5-methoxy-4-((2-(4-(methoxycarbonyl)phenyl)-4-phenylpiperazin-1-yl)methyl)-7-methyl-1H-indole-1-carboxylate was prepared with the following procedure. A Schlenk flask was charged with tert-butyl 5-methoxy-4-((2-(4-(methoxycarbonyl)phenyl)piperazin-1-yl)methyl)-7-methyl-1H-indole-1-carboxylate (50 mg, 1.0 eq, 0.10 mmol), RuPhos (3.9 mg, 0.08 eq, 8.4 μmol), Pd₂(dba)₃ (6.5 mg, 0.07 eq, 7.1 μmol), and NaOtBu (12 mg, 1.2 eq, 0.12 mmol) and purged with N₂ (3× vacuum/N2 cycles). Then, under a flow of N₂, bromobenzene (12 μL, 1.1 eq, 0.11 mmol) was added followed by THF (1 mL). The mixture was purged with N₂ (3× vacuum/N₂ cycles and heated in a pre-heated oil bath at 85° C. for 15 h. The reaction was worked-up; cooled to 20° C., filtered over Celite and the filtrate concentrated under reduced pressure. The residue was purified by automated reverse phase FCC to afford tert-butyl 5-methoxy-4-((2-(4-(methoxycarbonyl)phenyl)-4-phenylpiperazin-1-yl)methyl)-7-methyl-1H-indole-1-carboxylate (20 mg, 35%) as a yellow oil. LCMS (General 3) RT: 2.11 min; area % (254 nm): 83%; m/z=570.6 [M+H]⁺.

Step 2. 4-(1-((5-Methoxy-7-methyl-1H-indol-4-yl)methyl)-4-phenylpiperazin-2-yl)benzoic acid was prepared with the following procedure. To a suspension of tert-butyl 5-methoxy-4-((2-(4-(methoxycarbonyl)phenyl)-4-phenylpiperazin-1-yl)methyl)-7-methyl-1H-indole-1-carboxylate (16 mg, 1.0 eq, 28 μmol) in MeOH/H₂O (10:1, 1.1 mL) was added 15 wt. % aq. NaOH (64 μL, 10.0 eq, 0.14 mmol) and the mixture heated at 50° C. for 15 h. The reaction was worked-up; neutralized with 0.5 M aq. HCl and purified by automated reverse phase FCC to afford 4-(1-((5-methoxy-7-methyl-1H-indol-4-yl)methyl)-4-phenylpiperazin-2-yl)benzoic acid (3 mg, 20%) was obtained as a white solid. LCMS (31697 LCMS-5 C3) RT: 2.257 min; area % (215 nm): 97.1%; m/z=456.2 [M+H]⁺. ¹H-NMR (400 MHz, Methanol-d₄) δ 8.13 (d, J=8.1 Hz, 2H), 7.72 (d, J=8.3 Hz, 2H), 7.27 (d, J=3.1 Hz, 1H), 7.25-7.11 (m, 2H), 6.96 (t, J=9.1, 8.0 Hz, 2H), 6.85 (d, J=7.3 Hz, 1H), 6.74 (s, 1H), 6.41 (d, J=3.2 Hz, 1H), 4.13 (d, J=12.1 Hz, 2H), 3.78 (s, 3H), 3.71 (ddd, J=23.9, 13.2, 7.5 Hz, 3H), 3.15-2.84 (m, 3H), 2.59-2.37 (m, 3H).

### Example 8: 4-(1-((5-methoxy-7-methyl-1H-indol-4-yl)methyl)piperazin-2-yl)benzoic acid

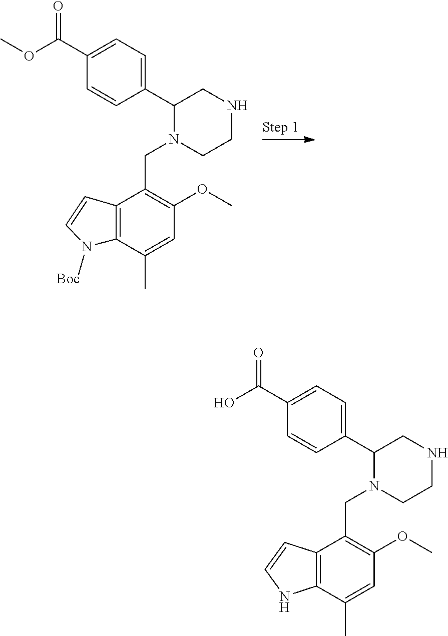

Step 1. 4-(1-((5-Methoxy-7-methyl-1H-indol-4-yl)methyl)piperazin-2-yl)benzoic acid was prepared with the following procedure. To a suspension of tert-butyl 5-methoxy-4-((2-(4-(methoxycarbonyl)phenyl)piperazin-1-yl)methyl)-7-methyl-1H-indole-1-carboxylate (25 mg, 1.0 eq, 51 μmol) in MeOH/H₂O (10:1, 1.1 mL) was added 15 wt. % aq. NaOH (12 μL, 1.0 eq, 51 μmol) and heated at 45° C. for 15 h. The reaction was worked-up; neutralized with 0.5 M aq. HCl and purified by automated reverse phase FCC to afford 4-(1-((5-methoxy-7-methyl-1H-indol-4-yl)methyl)piperazin-2-yl)benzoic acid (16.8 mg, 87%) as a white solid. LCMS (31697 LCMS-5 C3) RT: 1.305 min; area % (215 nm): >99%; m/z=380.2 [M+H]⁺. ¹H-NMR (400 MHz, DMSO-d₆) δ 10.85 (s, 1H), 7.97 (d, J=8.5 Hz, 2H), 7.61 (d, J=7.8 Hz, 2H), 7.25 (t, J=2.8 Hz, 1H), 6.66 (s, 1H), 6.40 (dd, J=3.1, 1.8 Hz, 1H), 3.71 (s, 3H), 3.57 (d, J=11.8 Hz, 1H), 3.40 (dd, J=10.7, 3.0 Hz, 1H), 3.24 (d, J=11.7 Hz, 1H), 2.96 (dd, J=17.5, 12.7 Hz, 2H), 2.84-2.71 (m, 1H), 2.71-2.59 (m, 2H), 2.47-2.33 (m, 3H), 2.29-2.14 (m, 1H).

### Example 9: 4-(4-Isopropyl-1-((5-methoxy-7-methyl-1H-indol-4-yl)methyl)piperazin-2-yl)benzoic acid

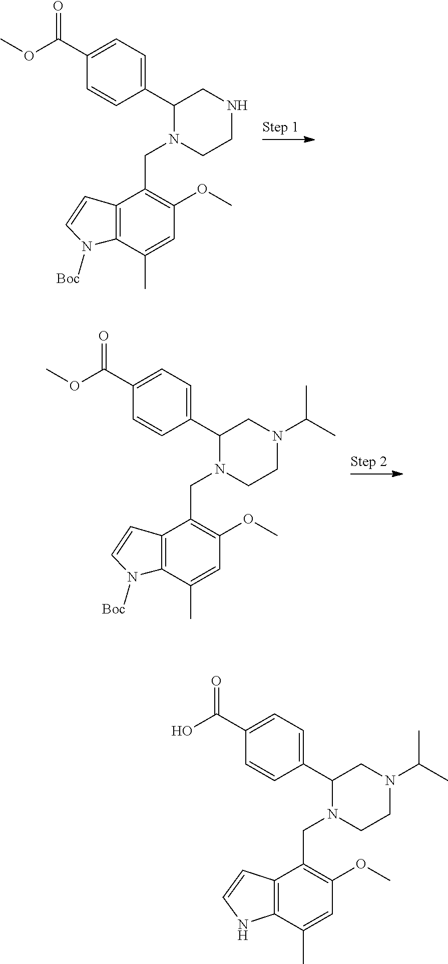

Step 1. tert-Butyl 4-((4-isopropyl-2-(4-(methoxycarbonyl)phenyl)piperazin-1-yl)methyl)-5-methoxy-7-methyl-1H-indole-1-carboxylate was prepared according to General Experimental Procedure 1. tert-Butyl 5-methoxy-4-((2-(4-(methoxycarbonyl)phenyl)piperazin-1-yl)methyl)-7-methyl-1H-indole-1-carboxylate (24 mg, 49 μmol) and acetone (15 μL, 0.20 mol) gave crude tert-Butyl 4-((4-isopropyl-2-(4-(methoxycarbonyl)phenyl)piperazin-1-yl)methyl)-5-methoxy-7-methyl-1H-indole-1-carboxylate.

Step 2. 4-(4-Isopropyl-1-((5-methoxy-7-methyl-1H-indol-4-yl)methyl)piperazin-2-yl)benzoic acid was prepared according to General Experimental Procedure 2. Crude tert-butyl 4-((4-isopropyl-2-(4-(methoxycarbonyl)phenyl)piperazin-1-yl)methyl)-5-methoxy-7-methyl-1H-indole-1-carboxylate gave 4-(4-isopropyl-1-((5-methoxy-7-methyl-1H-indol-4-yl)methyl)piperazin-2-yl)benzoic acid (11 mg, 51% over two steps) as a white solid. LCMS (31697 LCMS-5 C3) RT: 1.506 min; area % (215 nm): 98.7%; m/z=422.3 [M+H]⁺. ¹H-NMR (400 MHz, DMSO-d₆) δ 10.81 (t, J=2.3 Hz, 1H), 7.97 (d, J=8.5 Hz, 2H), 7.68 (d, J=7.8 Hz, 2H), 7.25 (t, J=2.8 Hz, 1H), 6.65 (s, 1H), 6.44 (dd, J=3.1, 1.9 Hz, 1H), 3.70 (s, 3H), 3.56 (d, J=11.8 Hz, 1H), 3.21 (d, J=11.8 Hz, 1H), 2.81-2.57 (m, 4H), 2.42 (s, 3H), 2.33 (q, J=1.9 Hz, 1H), 2.26-2.11 (m, 2H), 2.08 (s, 1H), 0.91 (d, J=6.5 Hz, 6H).

### Example 10: 4-(4-(Cyclopropylmethyl)-1-((5-methoxy-7-methyl-1H-indol-4-yl)methyl)piperazin-2-yl)benzoic acid

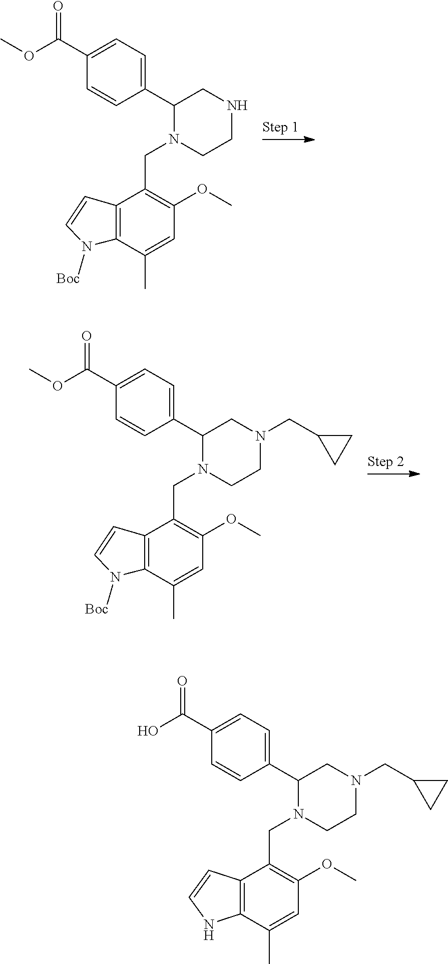

Step 1. tert-Butyl 4-((4-(cyclopropylmethyl)-2-(4-(methoxycarbonyl)phenyl)piperazin-1-yl)methyl)-5-methoxy-7-methyl-1H-indole-1-carboxylate was prepared according to General Experimental Procedure 1. tert-Butyl 5-methoxy-4-((2-(4-(methoxycarbonyl)phenyl)piperazin-1-yl)methyl)-7-methyl-1H-indole-1-carboxylate (24 mg, 49 μmol) and cyclopropancarboxaldehyde (15 μL, 0.20 mol) gave crude tert-butyl 4-((4-(cyclopropylmethyl)-2-(4-(methoxycarbonyl)phenyl)piperazin-1-yl)methyl)-5-methoxy-7-methyl-1H-indole-1-carboxylate.

Step 2. 4-(4-(Cyclopropylmethyl)-1-((5-methoxy-7-methyl-1H-indol-4-yl)methyl)piperazin-2-yl)benzoic acid was prepared according to General Experimental Procedure 2. Crude tert-butyl 4-((4-(cyclopropylmethyl)-2-(4-(methoxycarbonyl)phenyl)piperazin-1-yl)methyl)-5-methoxy-7-methyl-1H-indole-1-carboxylate gave 4-(4-(cyclopropylmethyl)-1-((5-methoxy-7-methyl-1H-indol-4-yl)methyl)piperazin-2-yl)benzoic acid (11 mg, 52% over two steps) as a white solid. LCMS (31697 LCMS-5 C3) RT: 1.654 min; area % (215 nm): 92.1%; m/z=434.3 [M+H]⁺. ¹H-NMR (400 MHz, DMSO-d₆) δ 10.82 (s, 1H), 7.97 (d, J=8.4 Hz, 2H), 7.68 (d, J=7.8 Hz, 2H), 7.25 (t, J=2.8 Hz, 1H), 6.65 (s, 1H), 6.45 (dd, J=3.1, 1.9 Hz, 1H), 3.71 (s, 3H), 3.56 (d, J=11.8 Hz, 1H), 3.25-3.10 (m, 1H), 2.84 (ddd, J=17.6, 8.3, 5.9 Hz, 2H), 2.73-2.59 (m, 2H), 2.42 (s, 3H), 2.39-2.27 (m, 1H), 2.22 (td, J=11.6, 2.7 Hz, 1H), 2.13 (d, J=6.5 Hz, 2H), 2.09-1.85 (m, 2H), 0.90-0.66 (m, 1H), 0.51-0.25 (m, 2H), 0.15-−0.08 (m, 2H).

### Example 11: 4-(4-Isobutyl-1-((5-methoxy-7-methyl-1H-indol-4-yl)methyl)piperazin-2-yl)benzoic acid

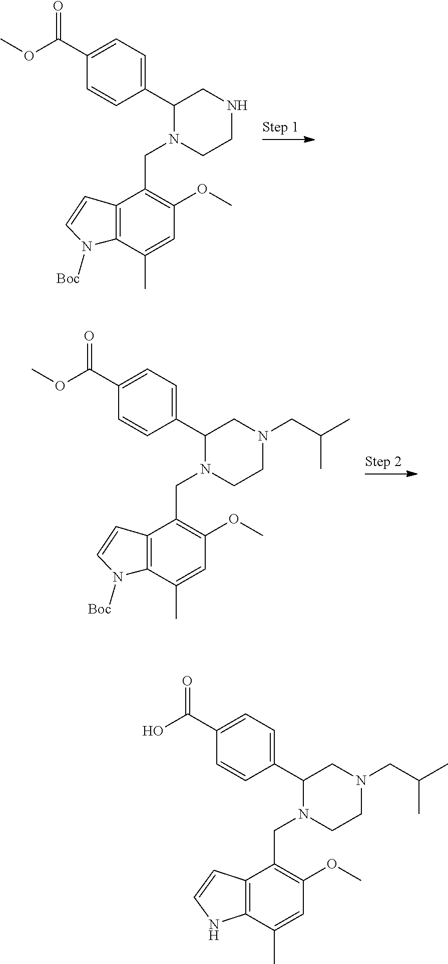

Step 1. tert-Butyl 4-((4-isobutyl-2-(4-(methoxycarbonyl)phenyl)piperazin-1-yl)methyl)-5-methoxy-7-methyl-1H-indole-1-carboxylate was prepared according to General Experimental Procedure 1. tert-Butyl 5-methoxy-4-((2-(4-(methoxycarbonyl)phenyl)piperazin-1-yl)methyl)-7-methyl-1H-indole-1-carboxylate (24 mg, 49 μmol) and isobutyraldehyde (17 μL, 0.20 mol) gave crude tert-butyl 4-((4-isobutyl-2-(4-(methoxycarbonyl)phenyl)piperazin-1-yl)methyl)-5-methoxy-7-methyl-1H-indole-1-carboxylate.

Step 2. 4-(4-Isobutyl-1-((5-methoxy-7-methyl-1H-indol-4-yl)methyl)piperazin-2-yl)benzoic acid was prepared according to General Experimental Procedure 2. Crude tert-butyl 4-((4-isobutyl-2-(4-(methoxycarbonyl)phenyl)piperazin-1-yl)methyl)-5-methoxy-7-methyl-1H-indole-1-carboxylate gave 4-(4-isobutyl-1-((5-methoxy-7-methyl-1H-indol-4-yl)methyl)piperazin-2-yl)benzoic acid (10 mg, 46% over two steps) as a white solid. LCMS (31697 LCMS-5 C3) RT: 1.966 min; area % (215 nm): >99%; m/z=436.4 [M+H]⁺. ¹H-NMR (400 MHz, DMSO-d₆) δ 10.82 (s, 1H), 7.96 (d, J=8.3 Hz, 2H), 7.68 (d, J=7.8 Hz, 2H), 7.25 (t, J=2.8 Hz, 1H), 6.65 (s, 1H), 6.43 (dd, J=3.1, 1.9 Hz, 1H), 3.71 (s, 3H), 3.56 (d, J=11.8 Hz, 1H), 3.48-3.11 (m, 35H), 2.86-2.59 (m, 3H), 2.42 (s, 3H), 2.33 (p, J=1.9 Hz, OH), 2.26-2.14 (m, 1H), 2.08 (s, OH), 2.04-1.92 (m, 3H), 1.92-1.77 (m, 1H), 1.69 (dt, J=13.6, 6.8 Hz, 1H), 0.82 (dd, J=6.5, 1.8 Hz, 6H).

### Example 12: 4-(4-Cyclobutyl-1-((5-methoxy-7-methyl-1H-indol-4-yl)methyl)piperazin-2-yl)benzoic acid

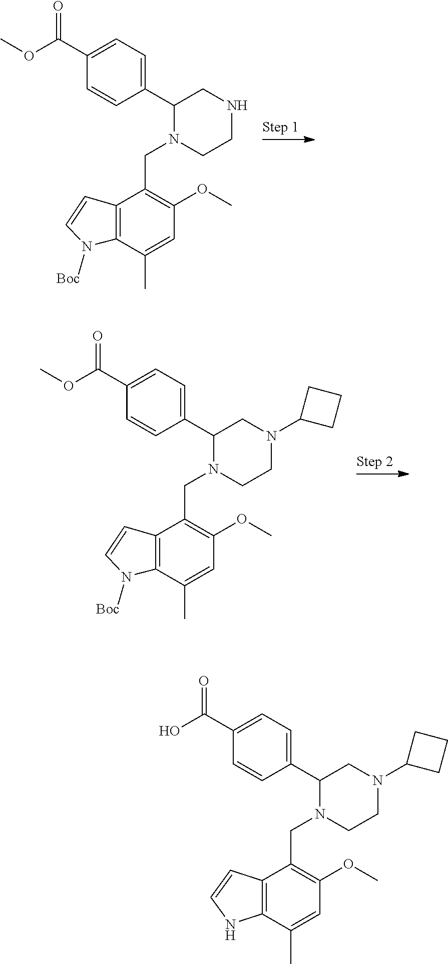

Step 1. tert-Butyl 4-((4-cyclobutyl-2-(4-(methoxycarbonyl)phenyl)piperazin-1-yl)methyl)-5-methoxy-7-methyl-1H-indole-1-carboxylate was prepared according to General Experimental Procedure 1. tert-Butyl 5-methoxy-4-((2-(4-(methoxycarbonyl)phenyl)piperazin-1-yl)methyl)-7-methyl-1H-indole-1-carboxylate (24 mg, 49 μmol) and cyclobutanone (15 μL, 0.20 mol) gave crude tert-butyl 4-((4-cyclobutyl-2-(4-(methoxycarbonyl)phenyl)piperazin-1-yl)methyl)-5-methoxy-7-methyl-1H-indole-1-carboxylate.

Step 2. 4-(4-Cyclobutyl-1-((5-methoxy-7-methyl-1H-indol-4-yl)methyl)piperazin-2-yl)benzoic acid was prepared according to General Experimental Procedure 2. Crude tert-butyl 4-((4-cyclobutyl-2-(4-(methoxycarbonyl)phenyl)piperazin-1-yl)methyl)-5-methoxy-7-methyl-1H-indole-1-carboxylate gave 4-(4-cyclobutyl-1-((5-methoxy-7-methyl-1H-indol-4-yl)methyl)piperazin-2-yl)benzoic acid (12 mg, 57% over two steps) as a white solid. LCMS (31697 LCMS-5 C3) RT: 1.734 min; area % (215 nm): 92.5%; m/z=434.4 [M+H]⁺. ¹H-NMR (400 MHz, DMSO-d₆) δ 10.82 (t, J=2.3 Hz, 1H), 7.96 (d, J=8.6 Hz, 2H), 7.67 (d, J=7.9 Hz, 2H), 7.25 (t, J=2.8 Hz, 1H), 6.65 (s, 1H), 6.43 (dd, J=3.1, 1.9 Hz, 1H), 3.70 (s, 3H), 3.57 (d, J=11.9 Hz, 1H), 3.22 (d, J=11.8 Hz, 1H), 2.74-2.60 (m, 4H), 2.51 (d, J=1.9 Hz, 2H), 2.41 (s, 3H), 2.18 (td, J=11.6, 2.8 Hz, 1H), 1.96-1.82 (m, 2H), 1.82-1.64 (m, 3H), 1.57 (dtd, J=18.2, 10.4, 8.9, 5.2 Hz, 2H).

### Example 13: 4-(1-((5-Methoxy-7-methyl-1H-indol-4-yl)methyl)-4-propylpiperazin-2-yl)benzoic

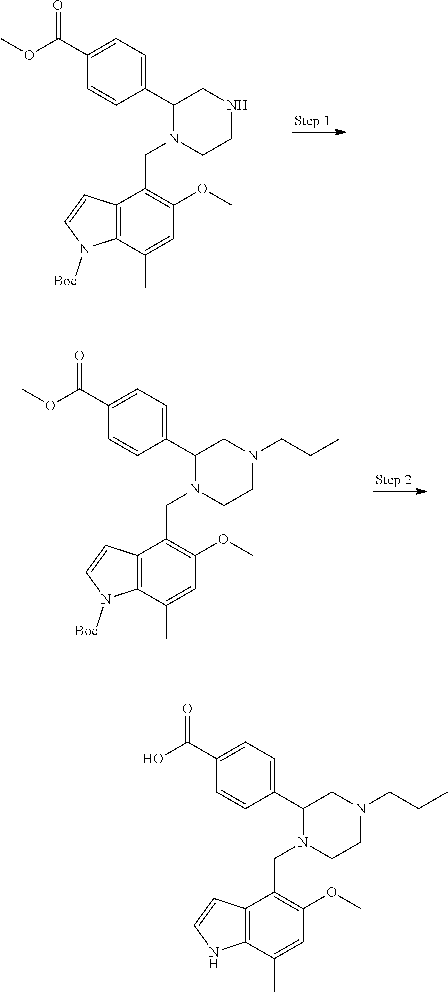

Step 1. tert-Butyl 5-methoxy-4-((2-(4-(methoxycarbonyl)phenyl)-4-propylpiperazin-1-yl)methyl)-7-methyl-1H-indole-1-carboxylate was prepared according to General Experimental Procedure 1. tert-Butyl 5-methoxy-4-((2-(4-(methoxycarbonyl)phenyl)piperazin-1-yl)methyl)-7-methyl-1H-indole-1-carboxylate (24 mg, 49 μmol) and propionaldehyde (15 μL, 0.20 mol) gave crude tert-butyl 5-methoxy-4-((2-(4-(methoxycarbonyl)phenyl)-4-propylpiperazin-1-yl)methyl)-7-methyl-1H-indole-1-carboxylate

Step 2. 4-(1-((5-Methoxy-7-methyl-1H-indol-4-yl)methyl)-4-propylpiperazin-2-yl)benzoic acid was prepared according to General Experimental Procedure 2. Crude tert-butyl 5-methoxy-4-((2-(4-(methoxycarbonyl)phenyl)-4-propylpiperazin-1-yl)methyl)-7-methyl-1H-indole-1-carboxylate gave 4-(1-((5-methoxy-7-methyl-1H-indol-4-yl)methyl)-4-propylpiperazin-2-yl)benzoic acid (7.8 mg, 38% over two steps) as a white solid. LCMS (31697 LCMS-5 C3) RT: 1.667 min; area % (215 nm): 92.0%; m/z=422.3 [M+H]⁺. ¹H-NMR (400 MHz, DMSO-d₆) δ 10.82 (t, J=2.3 Hz, 1H), 7.95 (d, J=8.1 Hz, 2H), 7.66 (d, J=7.6 Hz, 2H), 7.25 (t, J=2.8 Hz, 1H), 6.65 (s, 1H), 6.44 (dd, J=3.1, 1.9 Hz, 1H), 3.71 (s, 3H), 3.56 (d, J=11.8 Hz, 1H), 3.22 (d, J=11.8 Hz, 1H), 2.80-2.59 (m, 4H), 2.41 (s, 3H), 2.33 (p, J=1.8 Hz, 1H), 2.27-2.05 (m, 3H), 1.96 (t, J=10.7 Hz, 1H), 1.92-1.82 (m, 1H), 1.38 (h, J=7.2 Hz, 2H), 0.82 (t, J=7.3 Hz, 3H).

### Example 14: 4-(1-(1H-Indole-4-carbonyl)-4-methylpiperazin-2-yl)benzoic acid

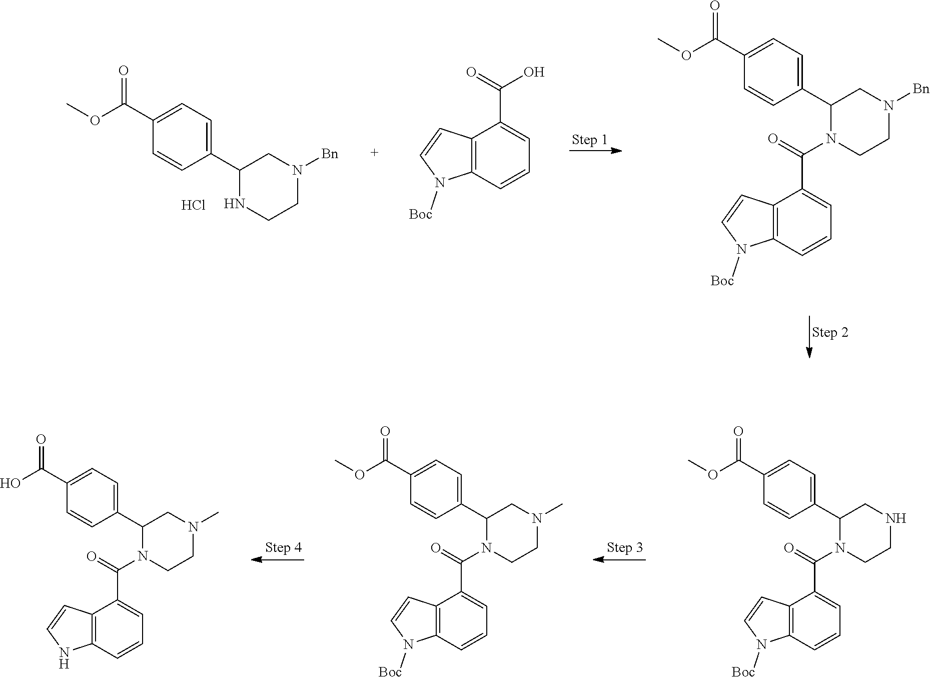

Step 1. tert-Butyl 4-(4-benzyl-2-(4-(methoxycarbonyl)phenyl)piperazine-1-carbonyl)-1H-indole-1-carboxylate was prepared with the following protocol. To a solution of methyl 4-(4-benzylpiperazin-2-yl)benzoate hydrochloride (139 mg, 1.03 eq, 401 μmol) and 1-(tert-butoxycarbonyl)-1H-indole-4-carboxylic acid (102 mg, 1 eq, 390 μmol) in DMF (3.0 mL) were added DIPEA (238 μL, 3.5 eq, 1.37 mmol) and HATU (223 mg, 1.5 eq, 586 μmol) The reaction mixture was stirred at 20° C. for 15 h. The reaction was worked-up; concentrated under reduced pressure and the residue was purified by reverse phase FCC to afford tert-butyl 4-(4-benzyl-2-(4-(methoxycarbonyl)phenyl)piperazine-1-carbonyl)-1H-indole-1-carboxylate (116 mg, 54%) as a clear oil. LCMS (General 3) RT: 1.85 min; area % (254 nm): 100%; m/z=554.4 [M+H]⁺.

Step 2. tert-Butyl 4-(2-(4-(methoxycarbonyl)phenyl)piperazine-1-carbonyl)-1H-indole-1-carboxylate was prepared with the following protocol. To a solution of tert-butyl 4-(4-benzyl-2-(4-(methoxycarbonyl)phenyl)piperazine-1-carbonyl)-1H-indole-1-carboxylate (116 mg, 1 eq, 210 μmol) in MeOH/AcOH (10:1 2.2 mL) was added 20% Pd(OH)₂ on carbon and the resulting suspension was hydrogenated at 70 psi in a pressure reactor at 20° C. for 15 h. The reaction was worked-up; filtered over Celite, the filter cake was washed with MeOH, and the filtrate concentrated under reduced pressure to afford crude tert-butyl 4-(2-(4-(methoxycarbonyl)phenyl)piperazine-1-carbonyl)-1H-indole-1-carboxylate (99 mg) was obtained as a pale yellow oil. LCMS (General 3) RT: 1.26 min; area % (254 nm): 98%; m/z=464.4 [M+H]⁺.

Step 3. tert-Butyl 4-(2-(4-(methoxycarbonyl)phenyl)-4-methylpiperazine-1-carbonyl)-1H-indole-1-carboxylate was prepared with the following protocol. To a solution of tert-butyl 4-(2-(4-(methoxycarbonyl)phenyl)piperazine-1-carbonyl)-1H-indole-1-carboxylate (99 mg, 1.0- eq, max. 210 μmol) in MeOH (7 mL) were added 37 wt. % aq. formaldehyde (62.5 μL, 4.0 eq, 840 μmol) and sodium triacetoxyborohydride (223 mg, 5.0 qq, 1.05 mmol). The mixture was stirred at 20° C. for 15 h. The reaction was worked-up; concentrated under reduced pressure to afford crude tert-butyl 4-(2-(4-(methoxycarbonyl)phenyl)-4-methylpiperazine-1-carbonyl)-1H-indole-1-carboxylate (106 mg) as a pale yellow oil. LCMS (General 3) RT: 1.54 min; area % (254 nm): 93%; m/z=478.4 [M+H]⁺.

Step 4. 4-(1-(1H-Indole-4-carbonyl)-4-methylpiperazin-2-yl)benzoic acid was prepared according to the following protocol. To a solution of crude tert-butyl 4-(2-(4-(methoxycarbonyl)phenyl)-4-methylpiperazine-1-carbonyl)-1H-indole-1-carboxylate (106 mg, 1.0 eq, max. 210 μmol) in MeOH/H₂O (5:1, 6 mL) was added 50 wt. % aq. NaOH (0.22 mL, 20 eq, 4.20 mmol) and the mixture stirred at 45° C. for 15 h. The reaction was worked-up; neutralized with 37 wt. % aq. HCl to pH 7 and partially concentrated under reduced pressure. The residue was diluted with DMSO (2 mL), filtered over cotton, and purified by reverse phase FCC to afford 4-(1-(1H-indole-4-carbonyl)-4-methylpiperazin-2-yl)benzoic acid (9.0 mg, 25 μmol, 12% over three steps) was obtained as a white solid. LCMS (31697 LCMS-5 C3) RT: 1.343 min, area %: 94.3%, m/z=364.2% [M+H]⁺. 1H-NMR (400 MHz, DMSO-d₆) δ 11.34 (t, J=2.3 Hz, 1H), 7.93 (s, 2H), 7.62 (s, 2H), 7.46 (d, J=8.2 Hz, 1H), 7.43 (t, J=2.8 Hz, 1H), 7.00 (d, J=7.2 Hz, 1H), 6.31 (s, 1H), 2.96 (t, J=12.3 Hz, 1H), 2.67 (p, J=1.9 Hz, OH), 2.56-2.52 (m, 2H), 2.44-2.27 (m, 1H), 2.20 (s, 3H), 1.93 (d, J=18.2 Hz, 1H).

### Example 15: 4-(4-Cyclopentyl-1-((5-methoxy-7-methyl-1H-indol-4-yl)methyl)piperazin-2-yl)benzoic acid

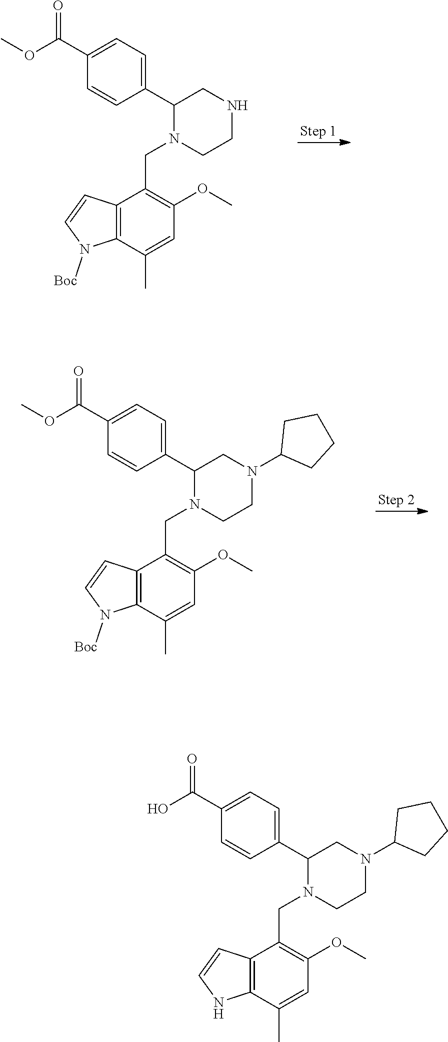

Step 1. tert-Butyl 4-((4-cyclopentyl-2-(4-(methoxycarbonyl)phenyl)piperazin-1-yl)methyl)-5-methoxy-7-methyl-1H-indole-1-carboxylate was prepared according to General Experimental Procedure 1. tert-Butyl 5-methoxy-4-((2-(4-(methoxycarbonyl)phenyl)piperazin-1-yl)methyl)-7-methyl-1H-indole-1-carboxylate (28 mg, 57 μmol) and cyclopentanone (20 μL, 0.23 mmol) gave crude tert-butyl 4-((4-cyclopentyl-2-(4-(methoxycarbonyl)phenyl)piperazin-1-yl)methyl)-5-methoxy-7-methyl-1H-indole-1-carboxylate.

Step 2. 4-(4-Cyclopentyl-1-((5-methoxy-7-methyl-1H-indol-4-yl)methyl)piperazin-2-yl)benzoic acid was prepared according to General Experimental Procedure 2. Crude tert-butyl 4-((4-cyclopentyl-2-(4-(methoxycarbonyl)phenyl)piperazin-1-yl)methyl)-5-methoxy-7-methyl-1H-indole-1-carboxylate gave 4-(4-cyclopentyl-1-((5-methoxy-7-methyl-1H-indol-4-yl)methyl)piperazin-2-yl)benzoic acid (13 mg, 51% over two steps) as a white solid. LCMS (31697 LCMS-5 C3) RT: 1.791 min; area % (215 nm): 98.5%; m/z=448.3 [M+H]⁺. ¹H-NMR (400 MHz, DMSO-d₆) δ 10.82 (t, J=2.3 Hz, 1H), 7.96 (d, J=8.4 Hz, 2H), 7.68 (d, J=7.8 Hz, 2H), 7.25 (t, J=2.8 Hz, 1H), 6.65 (s, 1H), 6.44 (dd, J=3.1, 1.9 Hz, 1H), 3.70 (s, 3H), 3.56 (d, J=11.8 Hz, 1H), 3.21 (d, J=11.8 Hz, 1H), 2.88-2.70 (m, 2H), 2.68-2.58 (m, 1H), 2.42 (s, 3H), 2.40-2.34 (m, 1H), 2.20 (td, J=11.6, 2.5 Hz, 1H), 2.04-1.86 (m, 2H), 1.77-1.61 (m, 2H), 1.61-1.50 (m, 2H), 1.50-1.36 (m, 2H), 1.36-1.20 (m, 2H).

### Example 16: 4-(4-(sec-Butyl)-1-((5-methoxy-7-methyl-1H-indol-4-yl)methyl)piperazin-2-yl)benzoic acid

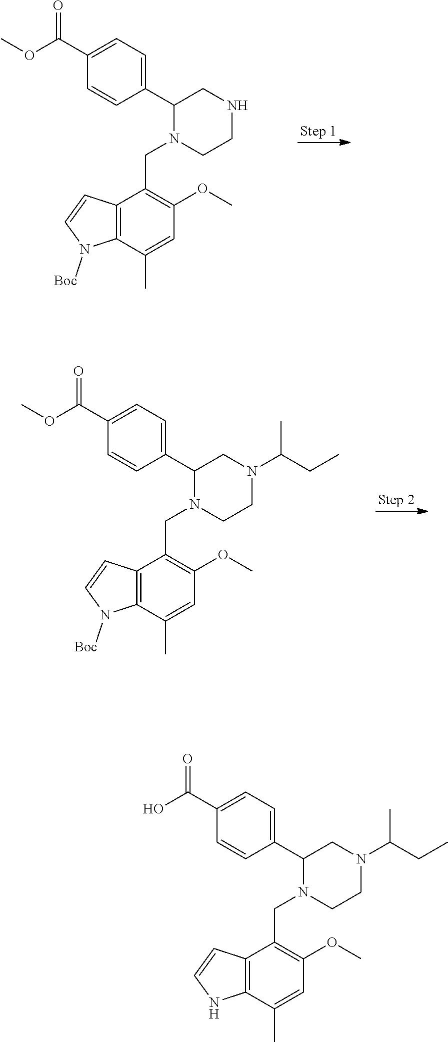

Step 1. tert-Butyl 4-((4-(sec-butyl)-2-(4-(methoxycarbonyl)phenyl)piperazin-1-yl)methyl)-5-methoxy-7-methyl-1H-indole-1-carboxylate was prepared according to General Experimental Procedure 1. tert-Butyl 5-methoxy-4-((2-(4-(methoxycarbonyl)phenyl)piperazin-1-yl)methyl)-7-methyl-1H-indole-1-carboxylate (28 mg, 57 μmol) and 2-butanone (20 μL, 0.23 mmol) gave crude tert-butyl 4-((4-(sec-butyl)-2-(4-(methoxycarbonyl)phenyl)piperazin-1-yl)methyl)-5-methoxy-7-methyl-1H-indole-1-carboxylate.

Step 2. 4-(4-(sec-Butyl)-1-((5-methoxy-7-methyl-1H-indol-4-yl)methyl)piperazin-2-yl)benzoic acid was prepared according to General Experimental Procedure 2. Crude tert-butyl 4-((4-(sec-butyl)-2-(4-(methoxycarbonyl)phenyl)piperazin-1-yl)methyl)-5-methoxy-7-methyl-1H-indole-1-carboxylate gave 4-(4-(sec-butyl)-1-((5-methoxy-7-methyl-1H-indol-4-yl)methyl)piperazin-2-yl)benzoic acid (16 mg, 64% over two steps) as a white solid. LCMS (31697 LCMS-5 C3) RT: 1.730 min; area % (215 nm): 98.0%; m/z=436.3 [M+H]⁺. ¹H-NMR (400 MHz, DMSO-d₆) δ 10.81 (t, J=2.3 Hz, 1H), 7.93 (d, J=8.5 Hz, 2H), 7.60 (d, J=7.9 Hz, 2H), 7.25 (t, J=2.8 Hz, 1H), 6.65 (s, 1H), 6.44 (ddd, J=2.9, 1.9, 0.8 Hz, 1H), 3.71 (s, 3H), 3.56 (d, J=11.8 Hz, 1H), 3.35-3.26 (m, 2H), 3.21 (d, J=11.8 Hz, 1H), 2.72-2.61 (m, 1H), 2.61-2.53 (m, 1H), 2.41 (s, 2H), 2.40-2.34 (m, 1H), 2.30-2.22 (m, 1H), 2.22-2.04 (m, 2H), 1.43 (dt, J=13.9, 7.1 Hz, 1H), 1.27-1.11 (m, 1H), 0.83 (ddd, J=8.8, 6.9, 1.9 Hz, 6H).

### Example 17: 4-(4-Butyl-1-((5-methoxy-7-methyl-1H-indol-4-yl)methyl)piperazin-2-yl)benzoic acid

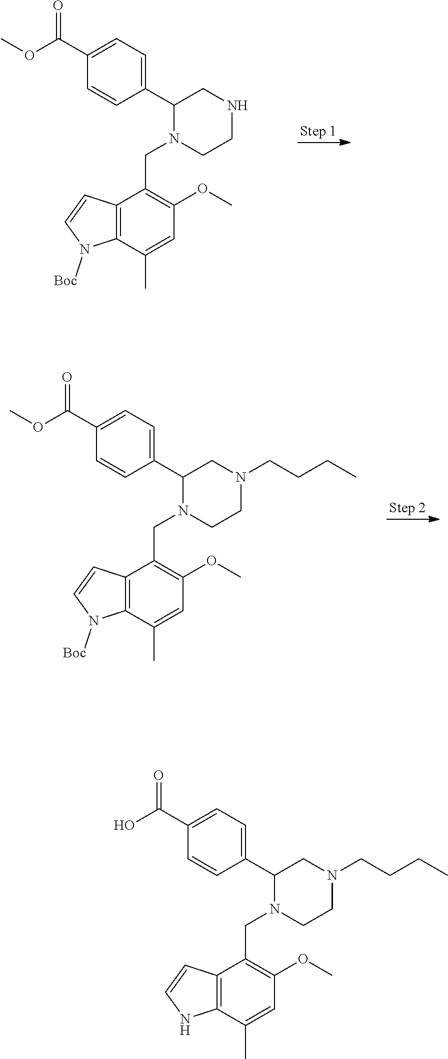

Step 1. tert-Butyl 4-((4-butyl-2-(4-(methoxycarbonyl)phenyl)piperazin-1-yl)methyl)-5-methoxy-7-methyl-1H-indole-1-carboxylate was prepared according to General Experimental Procedure 1. tert-Butyl 5-methoxy-4-((2-(4-(methoxycarbonyl)phenyl)piperazin-1-yl)methyl)-7-methyl-1H-indole-1-carboxylate (28 mg, 57 μmol) and butyraldehyde (21 μL, 0.23 mmol) gave crude tert-butyl 4-((4-butyl-2-(4-(methoxycarbonyl)phenyl)piperazin-1-yl)methyl)-5-methoxy-7-methyl-1H-indole-1-carboxylate.

Step 2. 4-(4-Butyl-1-((5-methoxy-7-methyl-1H-indol-4-yl)methyl)piperazin-2-yl)benzoic acid was prepared according to General Experimental Procedure 2. Crude tert-butyl 4-((4-butyl-2-(4-(methoxycarbonyl)phenyl)piperazin-1-yl)methyl)-5-methoxy-7-methyl-1H-indole-1-carboxylate gave 4-(4-butyl-1-((5-methoxy-7-methyl-1H-indol-4-yl)methyl)piperazin-2-yl)benzoic acid (7.7 mg, 31% over two steps) as a white solid. LCMS (31697 LCMS-5 C3) RT: 1.855 min; area % (215 nm): 97.3%; m/z=436.3 [M+H]⁺. ¹H-NMR (400 MHz, DMSO-d₆) δ 10.82 (t, J=2.3 Hz, 1H), 7.96 (d, J=8.6 Hz, 2H), 7.67 (d, J=7.7 Hz, 2H), 7.25 (t, J=2.8 Hz, 1H), 6.65 (s, 1H), 6.44 (dd, J=3.0, 1.9 Hz, 1H), 3.70 (s, 3H), 3.56 (d, J=11.8 Hz, 1H), 3.46-3.27 (m, 2H), 3.22 (d, J=11.8 Hz, 1H), 2.81-2.58 (m, 4H), 2.42 (s, 3H), 2.29-2.10 (m, 3H), 2.03-1.81 (m, 2H), 1.40-1.19 (m, 4H), 0.84 (t, J=7.2 Hz, 3H).

### Example 18: 4-(1-((5-Methoxy-7-methyl-1H-indol-4-yl)methyl)-4-(2-methylbutyl)piperazin-2-yl)benzoic acid

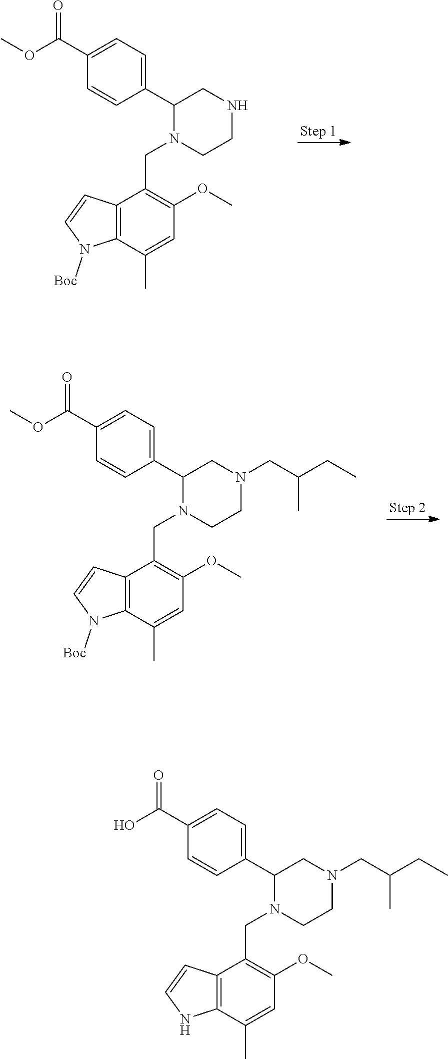

Step 1. tert-Butyl 5-methoxy-4-((2-(4-(methoxycarbonyl)phenyl)-4-(2-methylbutyl)piperazin-1-yl)methyl)-7-methyl-1H-indole-1-carboxylate was prepared according to General Experimental Procedure 1. tert-Butyl 5-methoxy-4-((2-(4-(methoxycarbonyl)phenyl)piperazin-1-yl)methyl)-7-methyl-1H-indole-1-carboxylate (28 mg, 57 μmol) and 2-methylbutanal (24 μL, 0.23 mmol) gave crude tert-butyl 5-methoxy-4-((2-(4-(methoxycarbonyl)phenyl)-4-(2-methylbutyl)piperazin-1-yl)methyl)-7-methyl-1H-indole-1-carboxylate.

Step 2. 4-(1-((5-Methoxy-7-methyl-1H-indol-4-yl)methyl)-4-(2-methylbutyl)piperazin-2-yl)benzoic acid was prepared according to General Experimental Procedure 2. Crude tert-butyl 5-methoxy-4-((2-(4-(methoxycarbonyl)phenyl)-4-(2-methylbutyl)piperazin-1-yl)methyl)-7-methyl-1H-indole-1-carboxylate gave 4-(1-((5-methoxy-7-methyl-1H-indol-4-yl)methyl)-4-(2-methylbutyl)piperazin-2-yl)benzoic acid (13 mg, 52% over two steps) as a white solid. LCMS (31697 LCMS-5 C3) RT: 2.172 min; area % (215 nm): >99%; m/z=450.4 [M+H]⁺. 1H NMR (400 MHz, DMSO) δ 10.82 (t, J=2.3 Hz, 1H), 7.94 (d, J=7.9 Hz, 2H), 7.61 (d, J=7.6 Hz, 2H), 7.25 (t, J=2.8 Hz, 1H), 6.65 (s, 1H), 6.43 (d, J=2.6 Hz, 1H), 3.71 (s, 3H), 3.56 (dd, J=11.7, 2.0 Hz, 1H), 3.45-3.29 (m, 2H), 3.22 (d, J=11.8 Hz, 1H), 2.79-2.57 (m, 3H), 2.41 (s, 3H), 2.20 (ddd, J=11.7, 8.5, 3.5 Hz, 1H), 2.12-2.01 (m, 1H), 2.01-1.91 (m, 1H), 1.91-1.87 (m, OH), 1.87-1.77 (m, 1H), 1.57-1.43 (m, 1H), 1.38 (dtd, J=12.4, 7.5, 4.7 Hz, 1H), 1.10-0.94 (m, 1H), 0.88-0.74 (m, 6H).

### Example 19: 4-(4-Isopentyl-1-((5-methoxy-7-methyl-1H-indol-4-yl)methyl)piperazin-2-yl)benzoic acid

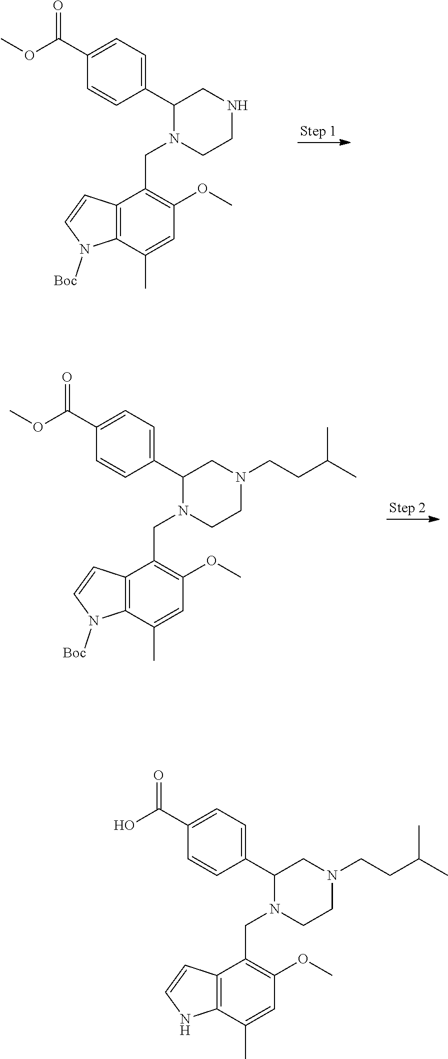

Step 1. tert-Butyl 4-((4-isopentyl-2-(4-(methoxycarbonyl)phenyl)piperazin-1-yl)methyl)-5-methoxy-7-methyl-1H-indole-1-carboxylate was prepared according to General Experimental Procedure 1. tert-Butyl 5-methoxy-4-((2-(4-(methoxycarbonyl)phenyl)piperazin-1-yl)methyl)-7-methyl-1H-indole-1-carboxylate (28 mg, 57 μmol) and 3-methylbutanal (24 μL, 0.23 mmol) gave crude tert-butyl 4-((4-isopentyl-2-(4-(methoxycarbonyl)phenyl)piperazin-1-yl)methyl)-5-methoxy-7-methyl-1H-indole-1-carboxylate.

Step 2. 4-(4-Isopentyl-1-((5-methoxy-7-methyl-1H-indol-4-yl)methyl)piperazin-2-yl)benzoic acid was prepared according to General Experimental Procedure 2. Crude tert-butyl 4-((4-isopentyl-2-(4-(methoxycarbonyl)phenyl)piperazin-1-yl)methyl)-5-methoxy-7-methyl-1H-indole-1-carboxylate gave 4-(4-isopentyl-1-((5-methoxy-7-methyl-1H-indol-4-yl)methyl)piperazin-2-yl)benzoic acid (6.8 mg, 30% over two steps) as a white solid. LCMS (31697 LCMS-5 C3) RT: 2.015 min; area % (215 nm): >99%; m/z=450.4 [M+H]⁺. ¹H-NMR (400 MHz, DMSO-d₆) δ 10.82 (t, J=2.2 Hz, 1H), 8.08-7.83 (m, 2H), 7.68 (d, J=7.8 Hz, 2H), 7.25 (t, J=2.8 Hz, 1H), 6.65 (s, 1H), 6.44 (dd, J=3.1, 1.9 Hz, 1H), 3.70 (s, 3H), 3.56 (d, J=11.8 Hz, 1H), 3.47-3.27 (m, 2H), 3.22 (d, J=11.8 Hz, 1H), 2.85-2.56 (m, 4H), 2.42 (s, 3H), 2.28-2.14 (m, 3H), 2.01-1.81 (m, 2H), 1.53 (hept, J=6.7 Hz, 1H), 1.26 (q, J=7.0 Hz, 2H), 0.83 (dd, J=6.6, 1.3 Hz, 6H).

### Example 20: 4-(4-(Cyclopentylmethyl)-1-((5-methoxy-7-methyl-1H-indol-4-yl)methyl)piperazin-2-yl)benzoic acid

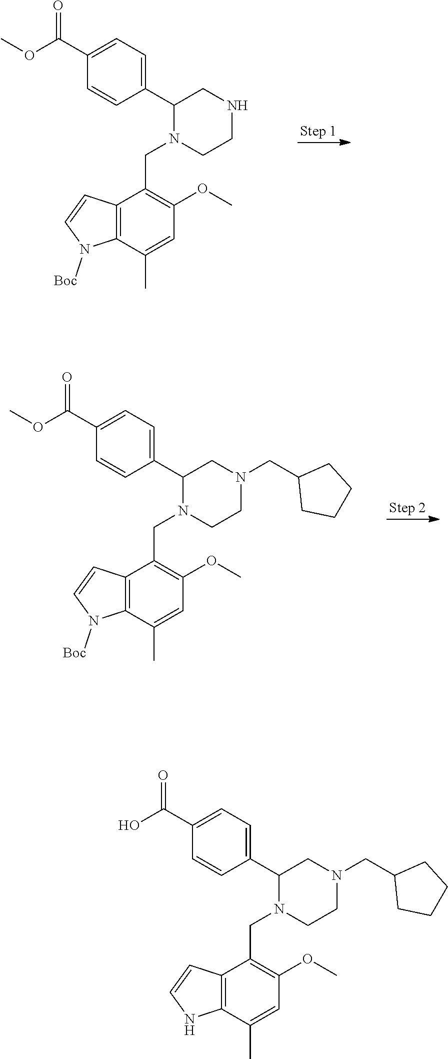

Step 1. tert-Butyl 4-((4-(cyclopentylmethyl)-2-(4-(methoxycarbonyl)phenyl)piperazin-1-yl)methyl)-5-methoxy-7-methyl-1H-indole-1-carboxylate was prepared according to General Experimental Procedure 1. tert-Butyl 5-methoxy-4-((2-(4-(methoxycarbonyl)phenyl)piperazin-1-yl)methyl)-7-methyl-1H-indole-1-carboxylate (28 mg, 57 μmol) and cyclopentanecarboxaldehyde (24 μL, 0.23 mmol) gave crude tert-butyl 4-((4-(cyclopentylmethyl)-2-(4-(methoxycarbonyl)phenyl)piperazin-1-yl)methyl)-5-methoxy-7-methyl-1H-indole-1-carboxylate.

Step 2. 4-(4-(Cyclopentylmethyl)-1-((5-methoxy-7-methyl-1H-indol-4-yl)methyl)piperazin-2-yl)benzoic acid was prepared according to General Experimental Procedure 2. Crude tert-butyl 4-((4-(cyclopentylmethyl)-2-(4-(methoxycarbonyl)phenyl)piperazin-1-yl)methyl)-5-methoxy-7-methyl-1H-indole-1-carboxylate gave 4-(4-(cyclopentylmethyl)-1-((5-methoxy-7-methyl-1H-indol-4-yl)methyl)piperazin-2-yl)benzoic acid (14 mg, 54% over two steps) as a white solid. LCMS (31697 LCMS-5 C3) RT: 2.117 min; area % (215 nm): 98.3%; m/z=462.3 [M+H]⁺. ¹H-NMR (400 MHz, DMSO-d₆) δ 10.82 (t, J=2.3 Hz, 1H), 8.04-7.84 (m, 2H), 7.63 (d, J=7.5 Hz, 2H), 7.25 (t, J=2.8 Hz, 1H), 6.65 (s, 1H), 6.43 (dd, J=3.1, 1.9 Hz, 1H), 3.70 (s, 3H), 3.56 (d, J=11.8 Hz, 1H), 3.45-3.28 (m, 2H), 3.22 (d, J=11.8 Hz, 1H), 2.77-2.68 (m, 2H), 2.62 (dt, J=11.7, 2.6 Hz, 1H), 2.41 (s, 3H), 2.20 (td, J=11.7, 2.7 Hz, 1H), 2.12 (dd, J=7.6, 2.2 Hz, 2H), 2.03-1.93 (m, 2H), 1.93-1.83 (m, 1H), 1.72-1.57 (m, 2H), 1.57-1.35 (m, 2H), 1.21-1.04 (m, 2H).

### Example 21: 4-(1-((5-Methoxy-7-methyl-1H-indol-4-yl)methyl)-4-(oxetan-3-yl)piperazin-2-yl)benzoic acid

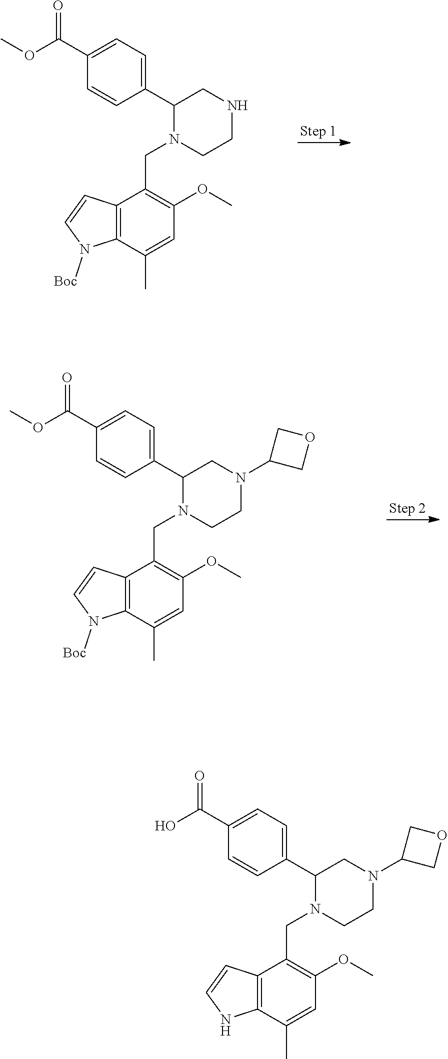

Step 1. tert-Butyl 5-methoxy-4-((2-(4-(methoxycarbonyl)phenyl)-4-(oxetan-3-yl)piperazin-1-yl)methyl)-7-methyl-1H-indole-1-carboxylate was prepared according to General Experimental Procedure 1. tert-Butyl 5-methoxy-4-((2-(4-(methoxycarbonyl)phenyl)piperazin-1-yl)methyl)-7-methyl-1H-indole-1-carboxylate (30 mg, 61 μmol) and 3-oxetanone (16 μL, 0.24 mmol) gave crude tert-butyl 5-methoxy-4-((2-(4-(methoxycarbonyl)phenyl)-4-(oxetan-3-yl)piperazin-1-yl)methyl)-7-methyl-1H-indole-1-carboxylate.

Step 2. 4-(1-((5-Methoxy-7-methyl-1H-indol-4-yl)methyl)-4-(oxetan-3-yl)piperazin-2-yl)benzoic acid was prepared according to General Experimental Procedure 2. Crude tert-butyl 5-methoxy-4-((2-(4-(methoxycarbonyl)phenyl)-4-(oxetan-3-yl)piperazin-1-yl)methyl)-7-methyl-1H-indole-1-carboxylate gave 4-(1-((5-methoxy-7-methyl-1H-indol-4-yl)methyl)-4-(oxetan-3-yl)piperazin-2-yl)benzoic acid (12 mg, 44% over two steps) as a white solid. LCMS (31697 LCMS-5 C3) RT: 1.523 min; area % (215 nm): 93.0%; m/z=436.2 [M+H]⁺. ¹H-NMR (400 MHz, DMSO-d₆) δ 10.83 (t, J=2.3 Hz, 1H), 7.96 (d, J=8.0 Hz, 2H), 7.66 (d, J=7.7 Hz, 2H), 7.25 (t, J=2.8 Hz, 1H), 6.66 (s, 1H), 6.43 (dd, J=3.0, 1.9 Hz, 1H), 4.54-4.29 (m, 4H), 3.71 (s, 3H), 3.58 (d, J=11.8 Hz, 1H), 3.42 (dd, J=10.2, 2.8 Hz, 1H), 3.35 (t, J=6.3 Hz, 1H), 3.24 (d, J=11.8 Hz, 1H), 2.79-2.56 (m, 2H), 2.42 (s, 3H), 2.24 (td, J=11.5, 2.5 Hz, 1H), 1.99-1.73 (m, 2H).

### Example 22: 4-(4-(3-Fluoropropyl)-1-((5-methoxy-7-methyl-1H-indol-4-yl)methyl)piperazin-2-yl)benzoic acid

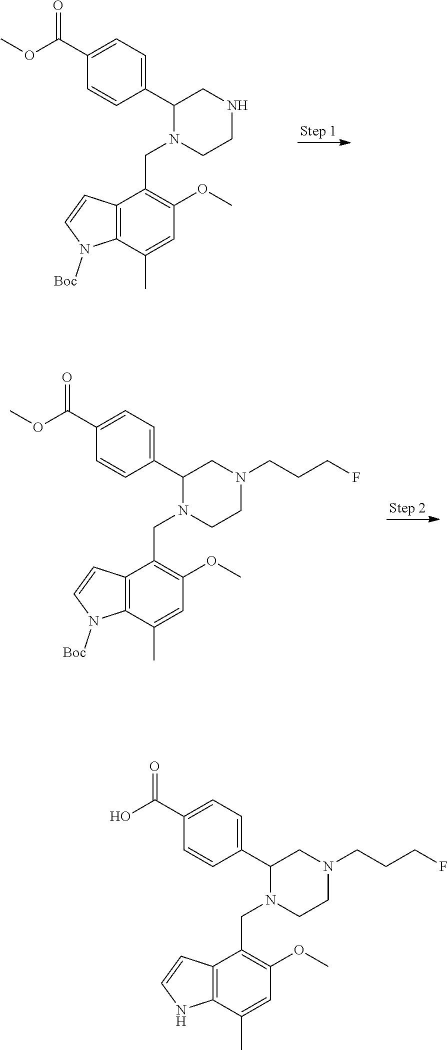

Step 1. tert-Butyl 4-((4-(3-fluoropropyl)-2-(4-(methoxycarbonyl)phenyl)piperazin-1-yl)methyl)-5-methoxy-7-methyl-1H-indole-1-carboxylate was prepared with the following procedure. A microwave vial was charged with tert-butyl 5-methoxy-4-((2-(4-(methoxycarbonyl)phenyl)piperazin-1-yl)methyl)-7-methyl-1H-indole-1-carboxylate (40 mg, 1.0 eq, 81 μmol) and THE (0.5 mL). To the solution were added Et₃N (12 μL, 1.1 eq, 89 μmol) and 1-fluoro-3-iodopropane (9.1 μL, 1.1 eq, 89 μmol). The microwave vial was capped and the reaction mixture heated in a pre-heated oil bath at 50° C. for 15 h. The reaction was worked-up; concentrated under reduced pressure and used crude residue as is in the subsequent step. LCMS (General 3) RT=1.90 min; area % (254 nm)=75%; m/z=554.5 [M+H]⁺.

Step 2. 4-(4-(3-Fluoropropyl)-1-((5-methoxy-7-methyl-1H-indol-4-yl)methyl)piperazin-2-yl)benzoic acid was prepared with the following procedure. To a solution of crude tert-butyl 4-((4-(3-fluoropropyl)-2-(4-(methoxycarbonyl)phenyl)piperazin-1-yl)methyl)-5-methoxy-7-methyl-1H-indole-1-carboxylate MeOH/H₂O (5:1, 1.2 mL) was added 50 wt. % aq. NaOH (26 μL, 6 eq, 0.49 mmol). The resulting mixture was heated at 45° C. for 15 h. The reaction was worked-up; neutralized with 37 wt. % aq. HCl and purified directly by reverse phase FCC to afford 4-(4-(3-fluoropropyl)-1-((5-methoxy-7-methyl-1H-indol-4-yl)methyl)piperazin-2-yl)benzoic acid (5.0 mg, 11 μmol, 14% over two steps) was obtained as a white solid. LCMS-5 (31697 LCMS-5 C3) RT: 1.678 min; area % (215 nm): 97.3%; m/z=440.3 [M+H]⁺. ¹H-NMR (400 MHz, DMSO-d₆) δ 10.82 (t, J=2.3 Hz, 1H), 7.97 (d, J=8.3 Hz, 2H), 7.69 (d, J=7.8 Hz, 2H), 7.25 (t, J=2.8 Hz, 1H), 6.66 (s, 1H), 6.43 (dd, J=3.1, 1.9 Hz, 1H), 4.51 (t, J=6.0 Hz, 1H), 4.39 (t, J=6.0 Hz, 1H), 3.71 (s, 3H), 3.56 (d, J=11.8 Hz, 1H), 3.44-3.36 (m, 4H), 3.23 (d, J=11.8 Hz, 1H), 2.80-2.68 (m, 2H), 2.68-2.59 (m, 1H), 2.42 (s, 3H), 2.38-2.25 (m, 2H), 2.21 (td, J=11.5, 2.5 Hz, 1H), 2.06-1.85 (m, 2H), 1.75 (dp, J=26.0, 6.7 Hz, 2H); ¹H-¹⁹F-coupling observed. ¹⁹F-NMR (376 MHz, DMSO-d₆) δ −218.37 (tt, J=47.5, 25.5 Hz); ¹⁹F-¹H-coupling observed.

### Example 23: 4-(4-Ethyl-1-((5-methoxy-7-methyl-1H-indol-4-yl)methyl)piperazin-2-yl)benzoic acid

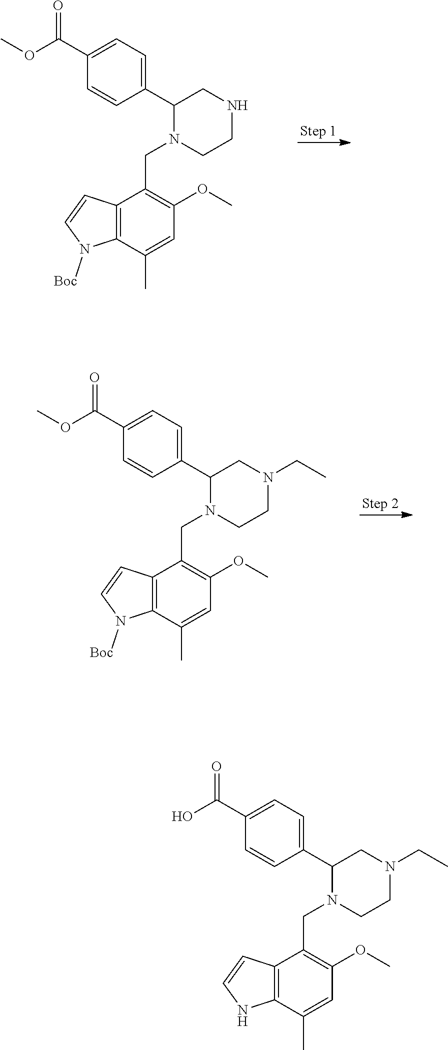

Step 1. tert-Butyl 4-((4-ethyl-2-(4-(methoxycarbonyl)phenyl)piperazin-1-yl)methyl)-5-methoxy-7-methyl-1H-indole-1-carboxylate was prepared according to General Experimental Procedure 1. tert-Butyl 5-methoxy-4-((2-(4-(methoxycarbonyl)phenyl)piperazin-1-yl)methyl)-7-methyl-1H-indole-1-carboxylate (37 mg, 1.0 eq, 75 μmol) and acetaldehyde (15 μL, 4.0 eq, 0.3 mmol) gave crude tert-butyl 4-((4-ethyl-2-(4-(methoxycarbonyl)phenyl)piperazin-1-yl)methyl)-5-methoxy-7-methyl-1H-indole-1-carboxylate. LCMS (General 3) RT=1.94 min; area % (254 nm)=64%; m/z=522.4 [M+H]⁺.

Step 2. 4-(4-Ethyl-1-((5-methoxy-7-methyl-1H-indol-4-yl)methyl)piperazin-2-yl)benzoic acid was prepared according to General Experimental Procedure 2. Crude tert-butyl 4-((4-ethyl-2-(4-(methoxycarbonyl)phenyl)piperazin-1-yl)methyl)-5-methoxy-7-methyl-1H-indole-1-carboxylate gave 4-(4-ethyl-1-((5-methoxy-7-methyl-1H-indol-4-yl)methyl)piperazin-2-yl)benzoic acid (10.8 mg, 35%) as a white solid. LCMS-5 (31697 LCMS-5 C3) RT: 1.458 min; area % (215 nm): 98.8%; m/z=408.4 [M+H]⁺. ¹H-NMR (400 MHz, DMSO-d₆) δ 10.82 (t, J=2.3 Hz, 1H), 8.10-7.86 (m, 2H), 7.68 (d, J=7.8 Hz, 2H), 7.25 (t, J=2.8 Hz, 1H), 6.65 (s, 1H), 6.44 (dd, J=3.1, 1.9 Hz, 1H), 3.71 (s, 3H), 3.56 (d, J=11.9 Hz, 1H), 3.39 (dd, J=10.2, 2.9 Hz, 1H), 3.22 (d, J=11.8 Hz, 1H), 2.81-2.69 (m, 2H), 2.69-2.59 (m, 1H), 2.42 (s, 3H), 2.31-2.14 (m, 3H), 2.03-1.81 (m, 2H), 0.94 (t, J=7.2 Hz, 3H).

### Example 24: Enantiomer 1 of 4-(1-((5-methoxy-7-methyl-1H-indol-4-yl)methyl)-4-methylpiperazin-2-yl)benzoic acid

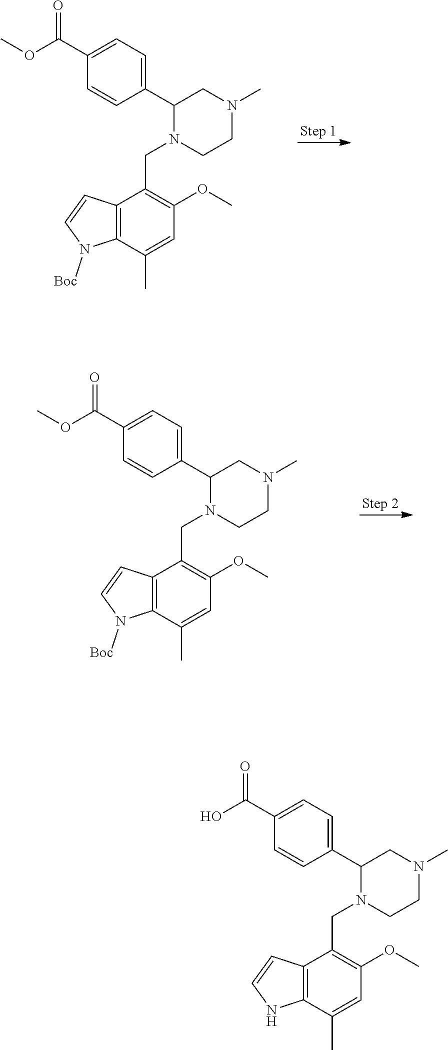

Step 1. Enantiomer 1 of tert-butyl 5-methoxy-4-((2-(4-(methoxycarbonyl)phenyl)-4-methylpiperazin-1-yl)methyl)-7-methyl-1H-indole-1-carboxylate was prepared with the following procedure. Racemic tert-butyl 5-methoxy-4-((2-(4-(methoxycarbonyl)phenyl)-4-methylpiperazin-1-yl)methyl)-7-methyl-1H-indole-1-carboxylate was separated in its enantiomers using preparative SFC (31697 EKN IG 5-30% 18 min). Crude enantiomer 1 of tert-butyl 5-methoxy-4-((2-(4-(methoxycarbonyl)phenyl)-4-methylpiperazin-1-yl)methyl)-7-methyl-1H-indole-1-carboxylate (10 mg) was obtained as an off-white solid that was used as is in the next step. First eluting enantiomer on chiral SFC (IG grad MA1) RT: 5.865 min; area % (254 nm): >99%; m/z=508.20 [M+H]⁺.

Step 2. Enantiomer 1 of 4-(1-((5-Methoxy-7-methyl-1H-indol-4-yl)methyl)-4-methylpiperazin-2-yl)benzoic acid was prepared with the following procedure. To a solution of crude enantiomer 1 of tert-butyl 5-methoxy-4-((2-(4-(methoxycarbonyl)phenyl)-4-methylpiperazin-1-yl)methyl)-7-methyl-1H-indole-1-carboxylate in MeOH/H₂O (10:1, 1 mL) was added 15 wt. % aq. NaOH (23 μL). The resulting solution was heated at 45° C. for 15 h. The reaction mixture was directly purified by reverse phase FCC to afford enantiomer 1 of 4-(1-((5-methoxy-7-methyl-1H-indol-4-yl)methyl)-4-methylpiperazin-2-yl)benzoic acid (5.1 mg) as a white solid. LCMS-5 (31697 LCMS-5 C3) RT: 1.363 min; area % (215 nm): >99%; m/z=394.3 [M+H]⁺. Chiral SFC (31697F UPC² 10m Cel2 MA1) RT=5.570 min; area % (220 nm): >99%; m/z=394.2 [M+H]⁺. ¹H-NMR was identical to that of Example 4.

### Example 25: Enantiomer 2 of 4-(1-((5-methoxy-7-methyl-1H-indol-4-yl)methyl)-4-methylpiperazin-2-yl)benzoic acid

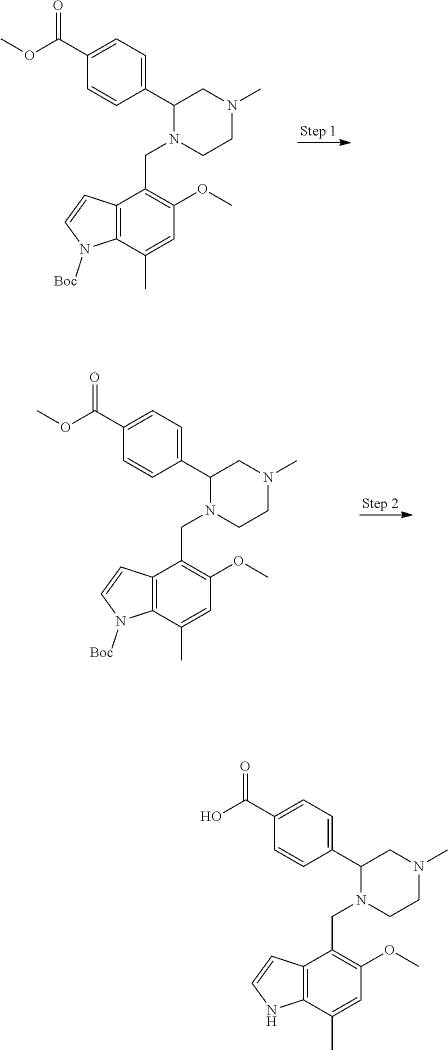

Step 1. Enantiomer 2 of tert-butyl 5-methoxy-4-((2-(4-(methoxycarbonyl)phenyl)-4-methylpiperazin-1-yl)methyl)-7-methyl-1H-indole-1-carboxylate was prepared with the following procedure. Racemic tert-butyl 5-methoxy-4-((2-(4-(methoxycarbonyl)phenyl)-4-methylpiperazin-1-yl)methyl)-7-methyl-1H-indole-1-carboxylate was separated in its enantiomers using preparative SFC (31697 EKN IG 5-30% 18 min). Crude enantiomer 2 of tert-butyl 5-methoxy-4-((2-(4-(methoxycarbonyl)phenyl)-4-methylpiperazin-1-yl)methyl)-7-methyl-1H-indole-1-carboxylate (7 mg) was obtained as an off-white solid that was used as is in the next step. Second eluting enantiomer on chiral SFC (IG grad MA1) RT: 6.526 min; area % (254 nm): >99%; m/z=508.2 [M+H]⁺.

Step 2. Enantiomer 2 of 4-(1-((5-Methoxy-7-methyl-1H-indol-4-yl)methyl)-4-methylpiperazin-2-yl)benzoic acid was prepared with the following procedure. To a solution of crude enantiomer 2 of tert-butyl 5-methoxy-4-((2-(4-(methoxycarbonyl)phenyl)-4-methylpiperazin-1-yl)methyl)-7-methyl-1H-indole-1-carboxylate in MeOH/H₂O (10:1, 1 mL) was added 15 wt. % aq. NaOH (16 μL). The resulting solution was heated at 45° C. for 15 h. The reaction mixture was directly purified by reverse phase FCC to afford enantiomer 2 of 4-(1-((5-methoxy-7-methyl-1H-indol-4-yl)methyl)-4-methylpiperazin-2-yl)benzoic acid (3.1 mg) as a white solid. LCMS-5 (31697 LCMS-5 C3) RT: 1.363 min; area % (215 nm): >99%; m/z=394.3 [M+H]⁺. Chiral SFC (31697F UPC² 10 m Cel2 MA1) RT=6.329 min; area % (220 nm): 98.6%; m/z=394.2 [M+H]⁺. ¹H-NMR was identical to that of Example 4.

### Example 26: 4-(4-Cyclopropyl-1-((5-methoxy-7-methyl-1H-indol-4-yl)methyl)piperazin-2-yl)benzoic acid

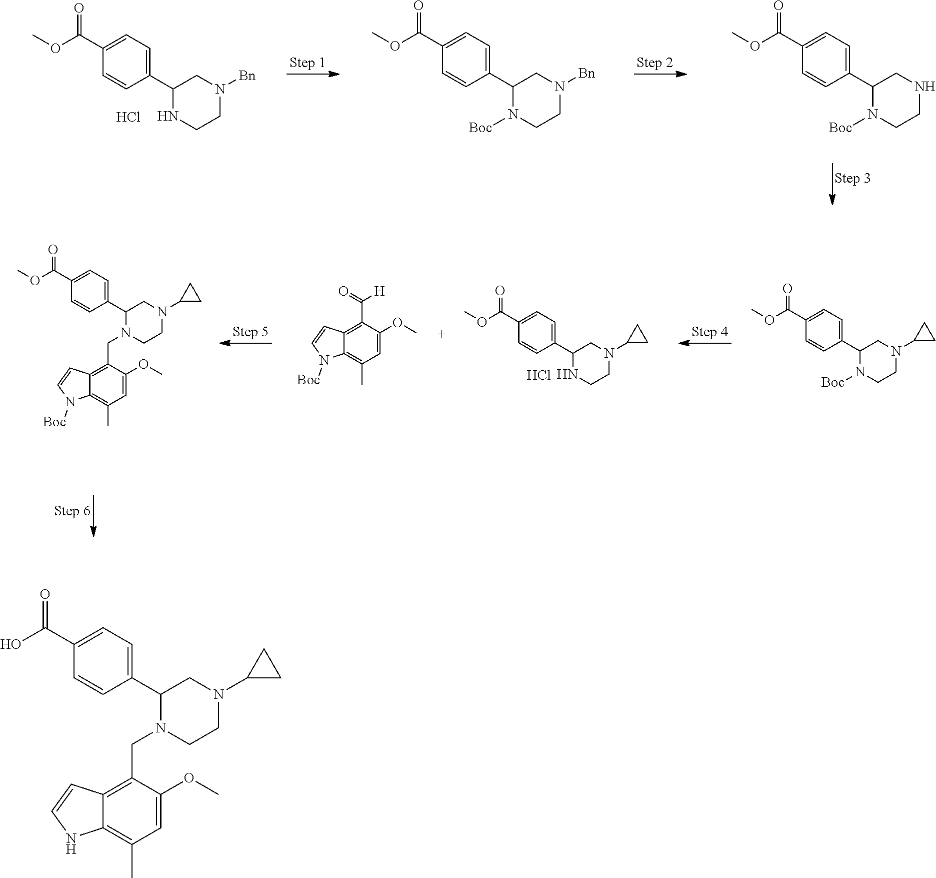

Step 1. tert-Butyl 4-benzyl-2-(4-(methoxycarbonyl)phenyl)piperazine-1-carboxylate was prepared with the following procedure. To a suspension of methyl 4-(4-benzylpiperazin-2-yl)benzoate hydrochloride (1.0 g, 1.0 eq, 2.9 mmol) in DCM (30 mL) under N2 was added Et3N (1.2 mL, 3.0 eq, 8.6 mmol). To the clear solution was added DMAP (35 mg, 0.1 Eq, 0.29 mmol) followed by Boc₂O (0.76 g, 0.79 mL, 1.2 Eq, 3.5 mmol). The resulting mixture was stirred at 20° C. for 15 h. The reaction mixture was coated on hydromatrix and purified by normal phase FCC to afford tert-butyl 4-benzyl-2-(4-(methoxycarbonyl)phenyl)piperazine-1-carboxylate (898 mg, 2.19 mmol, 76%) as a clear oil. LCMS (General 3) RT: 1.75 min; area % (254 nm): 100%; m/z=411.4 [M+H]⁺.

Step 2. tert-Butyl 2-(4-(methoxycarbonyl)phenyl)piperazine-1-carboxylate was prepared with the following procedure. To a solution of tert-butyl 4-benzyl-2-(4-(methoxycarbonyl)phenyl)piperazine-1-carboxylate (898 mg, 1.0 eq, 2.19 mmol) in MeOH (13 mL) was added 10% Pd/C (100 mg) as a suspension in MeOH (2 mL). The resulting suspension was hydrogenated at 58 psi in a pressure reactor at 20° C. for 15 h. The reaction was worked-up; filtered over Celite, the filter cake was washed with MeOH, and the filtrate concentrated under reduced pressure. tert-Butyl 2-(4-(methoxycarbonyl)phenyl)piperazine-1-carboxylate (700 mg, 99%) was obtained as a clear oil. LCMS (General 3) RT: 1.02 min; area % (254 nm): 95%; m/z=321.3 [M+H]⁺.

Step 3. tert-Butyl 4-cyclopropyl-2-(4-(methoxycarbonyl)phenyl)piperazine-1-carboxylate was prepared with the following procedure. To a suspension of tert-butyl 2-(4-(methoxycarbonyl)phenyl)piperazine-1-carboxylate (100 mg, 1.0 eq, 312 μmol), cyclopropylboronic acid (55.6 mg, 2.1 eq, 647 μmol), and Na₂CO₃ (66.7 mg, 2.02 eq, 629 μmol) in DCE (1 mL) was added a warm (−50° C.), slight suspension of 2,2′-bipyridine (49.3 mg, 1.01 eq, 316 μmol) and copper (II) acetate (56.7 mg, 1.0 eq, 312 μmol) in DCE (2 mL). The resulting mixture was heated at 70° C. for 5 h. The reaction was worked-up: cooled to room temperature and added 25 wt. % aq. NH₄OH (10 mL). Stirred vigorously for 30 min. The layers were separated and the aqueous layer extracted with CH₂Cl₂ (3×). The combined organic layers were dried over Na₂SO₄ and concentrated under reduced pressure. The residue was purified by reverse-phase FCC to afford tert-butyl 4-cyclopropyl-2-(4-(methoxycarbonyl)phenyl)piperazine-1-carboxylate (87 mg, 78%) was obtained as a pale yellow oil. LCMS (General3 LCMS-4 AB) RT=2.437 min, area % (254 nm): 74%, m/z=361.4 [M+H]⁺.

Step 4. Methyl 4-(4-cyclopropylpiperazin-2-yl)benzoate hydrochloride was prepared with the following procedure. To a solution of tert-butyl 4-cyclopropyl-2-(4-(methoxycarbonyl)phenyl)piperazine-1-carboxylate (87 mg, 1.0 eq, 242 μmol) in MeOH (5 mL) was added 4 N HCl in dioxane (302 μL, 5.0 eq, 1.21 mmol) and the mixture stirred at 65° C. for 15 h. The reaction was worked-up; concentrated under reduced pressure. To the residue was added Et₂O/acetone (10:1, 12 mL) and the solid triturated at room temperature for 1 h. The solid was collected by filtration, washed with Et₂O, and dried under reduced pressure. Methyl 4-(4-cyclopropylpiperazin-2-yl)benzoate hydrochloride (70 mg, 98.0%) was obtained as a beige solid. LCMS (General 3) RT=1.21 min, area % (254 nm): 100%, m/z=261.3 [M+H]⁺.

Step 5. tert-Butyl 4-((4-cyclopropyl-2-(4-(methoxycarbonyl)phenyl)piperazin-1-yl)methyl)-5-methoxy-7-methyl-1H-indole-1-carboxylate was prepared with the following procedure. To a suspension of methyl 4-(4-cyclopropylpiperazin-2-yl)benzoate hydrochloride (70 mg, 1.0 eq, 0.24 mmol) in THE (5.0 mL) was added Et3N (49 μL, 1.5 Eq, 0.35 mmol) and the mixture stirred at 20° C. for 15 min. Then tert-butyl 4-formyl-5-methoxy-7-methyl-1H-indole-1-carboxylate (67 mg, 0.98 eq, 0.23 mmol) and Ti(iOPr)₄ (140 μL, 2.0 eq, 0.47 mmol) were added and the mixture stirred at the same temperature for 1 h. Sodium triacetoxyborohydride (0.20 g, 4.0 eq, 0.94 mmol) was added and the mixture stirred at 20° C. for 15 h. Low conversion was observed. Additional tert-butyl 4-formyl-5-methoxy-7-methyl-1H-indole-1-carboxylate (35 mg, 0.5 eq, 0.12 mmol) and NaBH₄ (20 mg, 2 equiv.) were added. Stirred at 20° C. for 15 h. The reaction was worked-up; diluted with sat. aq. NaHCO₃/EtOAc, and filtered over Celite. The layers of the filtrate were separated and the aqueous layer extracted with EtOAc. The combined organic layers were washed with water, dried over Na₂SO₄, and concentrated under reduced pressure to afford crude tert-butyl 4-((4-cyclopropyl-2-(4-(methoxycarbonyl)phenyl)piperazin-1-yl)methyl)-5-methoxy-7-methyl-1H-indole-1-carboxylate (160 mg) as a pale yellow oil. LCMS (General 3) RT=2.08 min, area %: 51%, m/z=534.3 [M+H]⁺.

Step 6. 4-(4-Cyclopropyl-1-((5-methoxy-7-methyl-1H-indol-4-yl)methyl)piperazin-2-yl)benzoic acid was prepared with the following procedure. To a solution of crude tert-butyl 4-((4-cyclopropyl-2-(4-(methoxycarbonyl)phenyl)piperazin-1-yl)methyl)-5-methoxy-7-methyl-1H-indole-1-carboxylate (160 mg) in MeOH (3.0 mL)/H₂O (250 μL) was added 50 wt. % aq. NaOH (80 μL, 5.0 eq, 1.50 mmol) and the mixture heated at 45° C. for 15 h. The reaction was worked up; neutralized with the addition of 4 M aq. HCl to pH 7 and then purified by reverse phase FCC to afford 4-(4-cyclopropyl-1-((5-methoxy-7-methyl-1H-indol-4-yl)methyl)piperazin-2-yl)benzoic acid (59.6 mg, 59% over two steps) was obtained as a white solid. LCMS-5 (31697 LCMS-5 C3) RT: 1.824 min; area % (215 nm): >99%; m/z=420.4 [M+H]⁺. 1H-NMR (400 MHz, DMSO-d₆) δ 10.82 (t, J=2.3 Hz, 1H), 7.97 (d, J=8.5 Hz, 2H), 7.68 (d, J=7.7 Hz, 2H), 7.25 (t, J=2.8 Hz, 1H), 6.65 (d, J=0.9 Hz, 1H), 6.44 (dd, J=3.1, 1.9 Hz, 1H), 3.70 (s, 3H), 3.57 (d, J=11.8 Hz, 1H), 3.31 (dd, J=10.3, 3.0 Hz, 1H), 3.20 (d, J=11.8 Hz, 1H), 2.90-2.70 (m, 2H), 2.70-2.57 (m, 1H), 2.46-2.34 (m, 3H), 2.31-2.04 (m, 3H), 1.56 (tt, J=6.6, 3.6 Hz, 1H), 0.48-0.32 (m, 2H), 0.32-0.19 (m, 2H).

### Example 27: 4-(4-Allyl-1-((5-methoxy-7-methyl-1H-indol-4-yl)methyl)piperazin-2-yl)benzoic acid

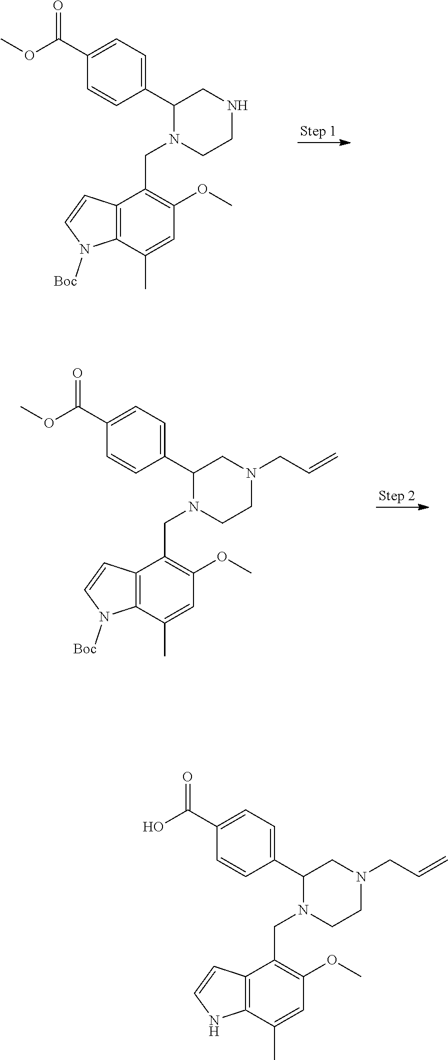

Step 1. tert-Butyl 4-((4-allyl-2-(4-(methoxycarbonyl)phenyl)piperazin-1-yl)methyl)-5-methoxy-7-methyl-1H-indole-1-carboxylate was prepared with the following procedure. To a solution of tert-butyl 5-methoxy-4-((2-(4-(methoxycarbonyl)phenyl)piperazin-1-yl)methyl)-7-methyl-1H-indole-1-carboxylate (40 mg, 1.0 eq, 81 μmol) in THE (1.0 mL) was added Et₃N (12 μL, 1.1 eq, 89 μmol) and stirred at 20° C. for 15 min. Then allylbromide (7.7 μL, 1.1 eq, 89 μmol) was added and the mixture stirred at 20° C. for 15 h. The reaction was worked up; the reaction mixture was concentrated under reduced pressure to afford crude tert-butyl 4-((4-allyl-2-(4-(methoxycarbonyl)phenyl)piperazin-1-yl)methyl)-5-methoxy-7-methyl-1H-indole-1-carboxylate. LCMS (General 3) RT=2.46 min; area % (254 nm)=92%; m/z=534.3 [M+H]⁺.

4-(4-Allyl-1-((5-methoxy-7-methyl-1H-indol-4-yl)methyl)piperazin-2-yl)benzoic acid was prepared according to General Experimental Procedure 2. Crude tert-butyl 4-((4-allyl-2-(4-(methoxycarbonyl)phenyl)piperazin-1-yl)methyl)-5-methoxy-7-methyl-1H-indole-1-carboxylate gave 4-(4-allyl-1-((5-methoxy-7-methyl-1H-indol-4-yl)methyl)piperazin-2-yl)benzoic acid (6.5 mg, 20% over two steps) as a white solid. LCMS-5 (31697 LCMS-5 C3) RT: 1.717 min; area % (215 nm): 92.3%; m/z=420.2 [M+H]⁺. ¹H-NMR (400 MHz, DMSO-d₆) δ 10.82 (s, 1H), 7.96 (d, J=8.4 Hz, 2H), 7.67 (d, J=7.8 Hz, 2H), 7.25 (t, J=2.8 Hz, 1H), 6.65 (s, 1H), 6.44 (dd, J=3.1, 1.9 Hz, 1H), 5.78 (ddt, J=16.7, 10.2, 6.3 Hz, 1H), 5.22-4.90 (m, 2H), 3.71 (s, 3H), 3.57 (d, J=11.9 Hz, 1H), 3.39 (dd, J=10.2, 2.9 Hz, 1H), 3.23 (d, J=11.9 Hz, 1H), 2.97-2.79 (m, 2H), 2.79-2.58 (m, 4H), 2.42 (s, 3H), 2.33 (p, J=1.8 Hz, 1H), 2.21 (td, J=11.5, 2.5 Hz, 1H), 2.07-1.86 (m, 2H).

### Example 28: 4-(1-((5-Methoxy-7-methyl-1H-indol-4-yl)methyl)-4-(prop-2-yn-1-yl)piperazin-2-yl)benzoic acid

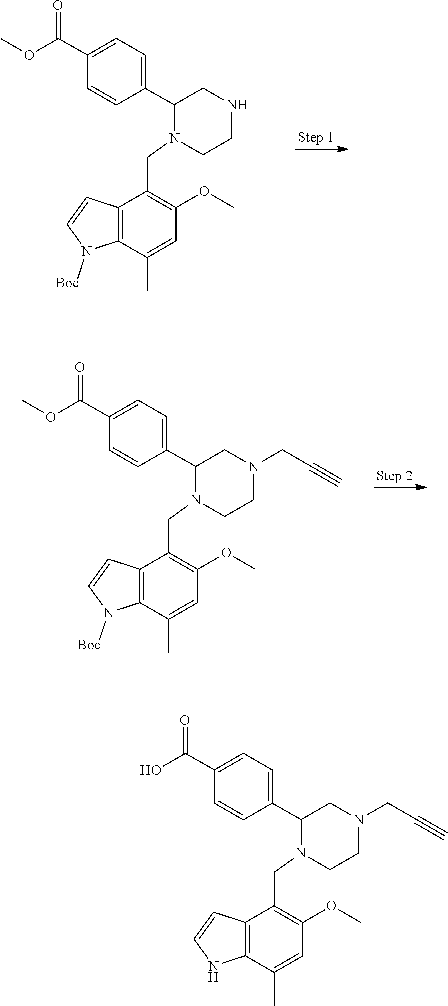

Step 1. tert-Butyl 5-methoxy-4-((2-(4-(methoxycarbonyl)phenyl)-4-(prop-2-yn-1-yl)piperazin-1-yl)methyl)-7-methyl-1H-indole-1-carboxylate was prepared with the following procedure. To a solution of tert-butyl 5-methoxy-4-((2-(4-(methoxycarbonyl)phenyl)piperazin-1-yl)methyl)-7-methyl-1H-indole-1-carboxylate (40 mg, 1.0 eq, 81 μmol) in THE (1.0 mL) was added Et₃N (12 μL, 1.1 eq, 89 μmol) and stirred at 20° C. for 15 min. Then 80 wt. % propargyl bromide in toluene (6.8 μL, 1.1 eq, 89 μmol) was added and the mixture stirred at 20° C. for 15 h. The reaction was worked up; the reaction mixture was concentrated under reduced pressure to afford crude tert-butyl 5-methoxy-4-((2-(4-(methoxycarbonyl)phenyl)-4-(prop-2-yn-1-yl)piperazin-1-yl)methyl)-7-methyl-1H-indole-1-carboxylate. LCMS (General 3) RT=2.31 min; area % (254 nm)=69%; m/z=532.3 [M+H]⁺.

4-(1-((5-Methoxy-7-methyl-1H-indol-4-yl)methyl)-4-(prop-2-yn-1-yl)piperazin-2-yl)benzoic acid was prepared according to General Experimental Procedure 2. Crude tert-butyl 5-methoxy-4-((2-(4-(methoxycarbonyl)phenyl)-4-(prop-2-yn-1-yl)piperazin-1-yl)methyl)-7-methyl-1H-indole-1-carboxylate gave 4-(1-((5-Methoxy-7-methyl-1H-indol-4-yl)methyl)-4-(prop-2-yn-1-yl)piperazin-2-yl)benzoic acid (4.7 mg, 14% over two steps) as a white solid. LCMS (31697 LCMS-5 C3) RT: 1.700 min; area % (215 nm): 93.5%; m/z=418.2 [M+H]⁺. ¹H-NMR (400 MHz, DMSO-d₆) δ 10.83 (s, 1H), 7.98 (d, J=8.5 Hz, 2H), 7.68 (d, J=7.7 Hz, 2H), 7.26 (t, J=2.8 Hz, 1H), 6.66 (s, 1H), 6.44 (dd, J=3.1, 1.9 Hz, 1H), 3.71 (s, 3H), 3.57 (d, J=11.9 Hz, 1H), 3.40 (dd, J=10.3, 3.0 Hz, 2H), 3.24 (s, 2H), 3.21 (s, 1H), 3.17-3.02 (m, 1H), 2.73-2.57 (m, 4H), 2.52 (d, J=1.9 Hz, 1H), 2.42 (s, 3H), 2.33 (t, J=1.9 Hz, 1H), 2.30-2.06 (m, 3H).

### Example 29: 4-(4-(2-Hydroxyethyl)-1-((5-methoxy-7-methyl-1H-indol-4-yl)methyl)piperazin-2-yl)benzoic acid

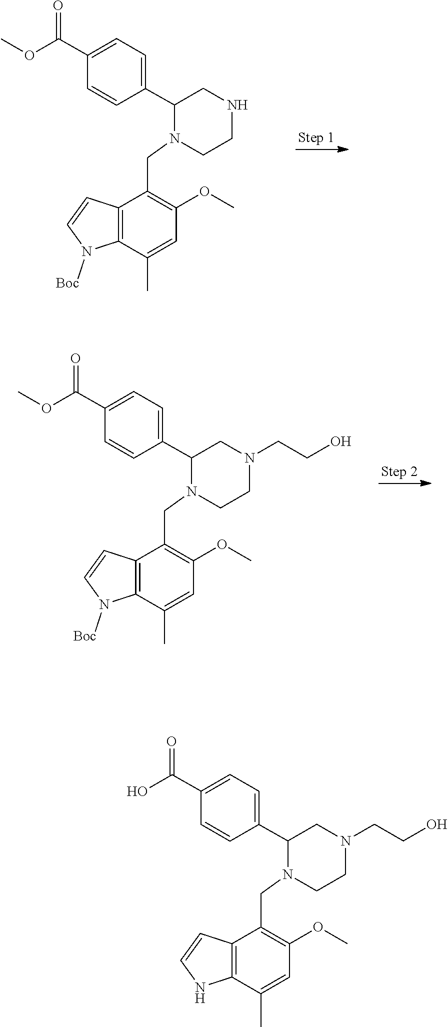

Step 1. tert-Butyl 4-((4-(2-hydroxyethyl)-2-(4-(methoxycarbonyl)phenyl)piperazin-1-yl)methyl)-5-methoxy-7-methyl-1H-indole-1-carboxylate was prepared with the following procedure. To a suspension of tert-butyl 5-methoxy-4-((2-(4-(methoxycarbonyl)phenyl)piperazin-1-yl)methyl)-7-methyl-1H-indole-1-carboxylate (50.0 mg, 1.0 eq, 101 μmol) and K₂CO₃ (27 mg, 2.0 eq, 0.20 mmol) in CH₃CN (1.5 mL) under N₂ was added 2-bromoethanol (7.0 μL, 1.0 eq, 99 μmol). The resulting mixture was heated at 85° C. for 15 h. Incomplete conversion was observed; additional K₂CO₃ (15 mg, 1.0 eq) and bromoethanol (21 μL, 3.0 eq.) were added. The reaction mixture was continued to heat at 85° C. for 15 h. The reaction was worked up; filtered over Celite and concentrated the filtrate under reduced pressure. The crude purified by automated reverse phase FCC to afford tert-butyl 4-((4-(2-hydroxyethyl)-2-(4-(methoxycarbonyl)phenyl)piperazin-1-yl)methyl)-5-methoxy-7-methyl-1H-indole-1-carboxylate (General 3) RT=2.04 min; area % (254 nm)=94%; m/z=538.3 [M+H]⁺.

4-(4-(2-Hydroxyethyl)-1-((5-methoxy-7-methyl-1H-indol-4-yl)methyl)piperazin-2-yl)benzoic acid was prepared according to General Experimental Procedure 2. Crude tert-butyl 4-((4-(2-hydroxyethyl)-2-(4-(methoxycarbonyl)phenyl)piperazin-1-yl)methyl)-5-methoxy-7-methyl-1H-indole-1-carboxylate gave 4-(4-(2-hydroxyethyl)-1-((5-methoxy-7-methyl-1H-indol-4-yl)methyl)piperazin-2-yl)benzoic acid (1.58 mg, 4% over two steps) as a white solid. LCMS-5 (31697 LCMS-5 C3) RT: 1.297 min; area % (215 nm): 98.5%; m/z=424.2 [M+H]⁺. ¹H-NMR (400 MHz, DMSO-d₆) δ 10.82 (s, 1H), 7.95 (d, J=7.9 Hz, 2H), 7.64 (d, J=7.1 Hz, 2H), 7.25 (t, J=2.8 Hz, 1H), 6.65 (s, 1H), 6.44 (dd, J=3.1, 1.9 Hz, 1H), 4.33 (s, 1H), 3.71 (s, 3H), 3.56 (d, J=11.9 Hz, 1H), 3.45 (t, J=6.2 Hz, 2H), 3.38 (dd, J=10.2, 2.8 Hz, 1H), 3.21 (d, J=11.8 Hz, 1H), 2.91-2.70 (m, 2H), 2.67 (t, J=1.9 Hz, 2H), 2.64-2.57 (m, 1H), 2.41 (s, 3H), 2.37-2.27 (m, 4H), 2.21 (td, J=11.5, 2.6 Hz, 1H), 2.10-1.93 (m, 2H).

### Example 30: 4-(4-(Cyclobutylmethyl)-1-((5-methoxy-7-methyl-1H-indol-4-yl)methyl)piperazin-2-yl)benzoic acid

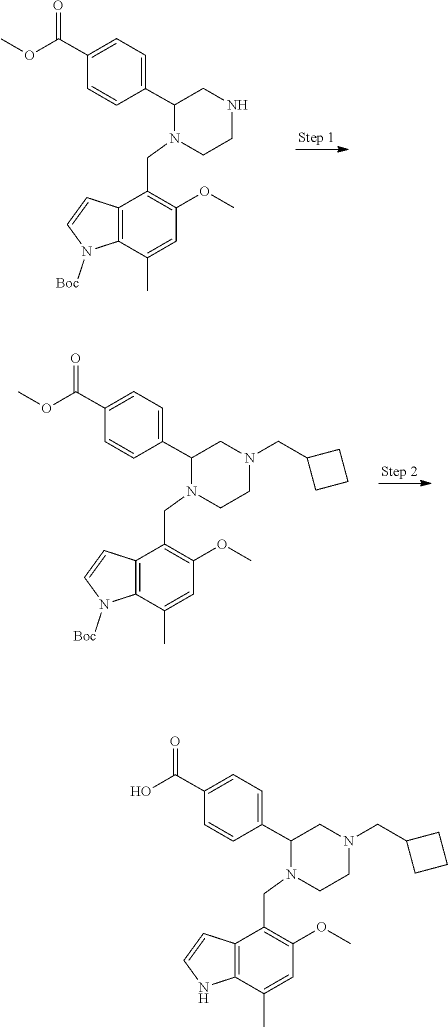

Step 1. tert-Butyl 4-((4-(cyclobutylmethyl)-2-(4-(methoxycarbonyl)phenyl)piperazin-1-yl)methyl)-5-methoxy-7-methyl-1H-indole-1-carboxylate was prepared according to General Experimental Procedure 1. tert-Butyl 5-methoxy-4-((2-(4-(methoxycarbonyl)phenyl)piperazin-1-yl)methyl)-7-methyl-1H-indole-1-carboxylate (30 mg, 1.0 eq, 61 μmol) and cyclobutanecarbaldehyde (20 mg, 4.0 eq, 0.24 mmol) gave crude tert-butyl 4-((4-(cyclobutylmethyl)-2-(4-(methoxycarbonyl)phenyl)piperazin-1-yl)methyl)-5-methoxy-7-methyl-1H-indole-1-carboxylate. (General 3) RT=2.31 min; area % (254 nm)=53%; m/z=562.3 [M+H]⁺.

4-(4-(Cyclobutylmethyl)-1-((5-methoxy-7-methyl-1H-indol-4-yl)methyl)piperazin-2-yl)benzoic acid was prepared according to General Experimental Procedure 2. Crude tert-butyl 4-((4-(cyclobutylmethyl)-2-(4-(methoxycarbonyl)phenyl)piperazin-1-yl)methyl)-5-methoxy-7-methyl-1H-indole-1-carboxylate gave 4-(4-(cyclobutylmethyl)-1-((5-methoxy-7-methyl-1H-indol-4-yl)methyl)piperazin-2-yl)benzoic acid (2.11 mg, 8% over two steps) as a white solid. LCMS-5 (31697 LCMS-5 C3) RT: 1.899 min; area % (215 nm): 93.8%; m/z=448.4 [M+H]⁺. ¹H-NMR (400 MHz, DMSO-d₆) δ 10.81 (s, 1H), 7.96 (d, J=8.0 Hz, 2H), 7.67 (d, J=7.8 Hz, 2H), 7.25 (t, J=2.8 Hz, 1H), 6.65 (s, 1H), 6.43 (t, J=2.4 Hz, 1H), 3.70 (s, 3H), 3.55 (d, J=11.8 Hz, 2H), 3.47-3.27 (m, 1H), 3.21 (d, J=11.8 Hz, 2H), 2.81-2.56 (m, 6H), 2.41 (s, 3H), 2.33 (dd, J=4.1, 2.2 Hz, 1H), 2.31-2.23 (m, 2H), 2.23-2.10 (m, 1H), 1.83-1.68 (m, 2H), 1.68-1.46 (m, 2H).

### Example 31: 4-(4-(2-Fluoroethyl)-1-((5-methoxy-7-methyl-1H-indol-4-yl)methyl)piperazin-2-yl)benzoic acid

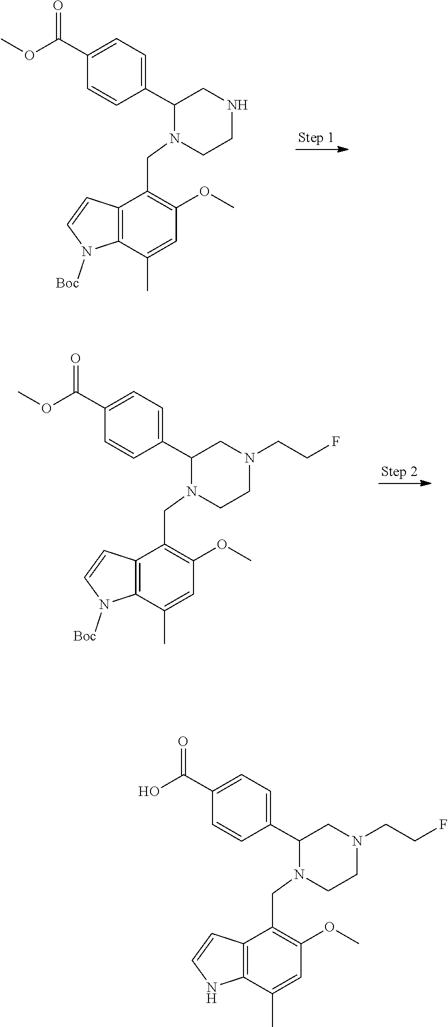

Step 1. tert-Butyl 4-((4-(2-fluoroethyl)-2-(4-(methoxycarbonyl)phenyl)piperazin-1-yl)methyl)-5-methoxy-7-methyl-1H-indole-1-carboxylate was prepared with the following procedure. To a solution of tert-butyl 5-methoxy-4-((2-(4-(methoxycarbonyl)phenyl)piperazin-1-yl)methyl)-7-methyl-1H-indole-1-carboxylate (40 mg, 1.0 eq, 81 μmol) in THE (1 mL) was added Et₃N (12 μL, 1.1 Eq, 86 μmol). The mixture was allowed to stir at 20° C. for 15 minutes. Then 1-fluoro-2-iodo-ethane (10 μL, 1.1 Eq, 85 μmol) was added and the subsequent mixture was stirred at 20° C. for 15 h. Incomplete conversion observed. The reaction mixture was heated at 50° C. for 15 h. The reaction was worked up; concentrated under reduced pressure to afford crude tert-butyl 4-((4-(2-fluoroethyl)-2-(4-(methoxycarbonyl)phenyl)piperazin-1-yl)methyl)-5-methoxy-7-methyl-1H-indole-1-carboxylate. (General 3) RT=2.04 min; area % (254 nm)=55%; m/z=540.4 [M+H]⁺.

4-(4-(2-Fluoroethyl)-1-((5-methoxy-7-methyl-1H-indol-4-yl)methyl)piperazin-2-yl)benzoic acid was prepared according to General Experimental Procedure 2. Crude tert-butyl 4-((4-(2-fluoroethyl)-2-(4-(methoxycarbonyl)phenyl)piperazin-1-yl)methyl)-5-methoxy-7-methyl-1H-indole-1-carboxylate gave 4-(4-(2-fluoroethyl)-1-((5-methoxy-7-methyl-1H-indol-4-yl)methyl)piperazin-2-yl)benzoic acid (3.47 mg, 10% over two steps) as a white solid. LCMS-5 (31697 LCMS-5 C3) RT: 1.572 min; area % (215 nm): 96.6%; m/z=426.2 [M+H]⁺. ¹H-NMR (400 MHz, DMSO-d₆) δ 10.82 (s, 1H), 7.97 (d, J=8.3 Hz, 2H), 7.68 (d, J=7.8 Hz, 2H), 7.26 (t, J=2.8 Hz, 1H), 6.66 (s, 1H), 6.44 (dd, J=3.1, 1.9 Hz, 1H), 4.55 (t, J=4.9 Hz, 1H), 4.43 (t, J=4.9 Hz, 1H), 3.71 (s, 3H), 3.57 (d, J=11.8 Hz, 1H), 3.41 (dd, J=10.3, 2.9 Hz, 3H), 3.23 (d, J=11.8 Hz, 2H), 2.93-2.70 (m, 2H), 2.70-2.64 (m, 1H), 2.64-2.58 (m, 3H), 2.42 (s, 3H), 2.33 (p, J=1.9 Hz, 1H), 2.22 (td, J=11.6, 2.7 Hz, 1H), 2.16-1.97 (m, 2H); ¹H-¹⁹F-coupling observed. ¹⁹F-NMR-HDEC (376 MHz, DMSO-d₆) δ −217.19.

### Example 32: 4-(4-(2,2-Difluoroethyl)-1-((5-methoxy-7-methyl-1H-indol-4-yl)methyl)piperazin-2-yl)benzoic acid

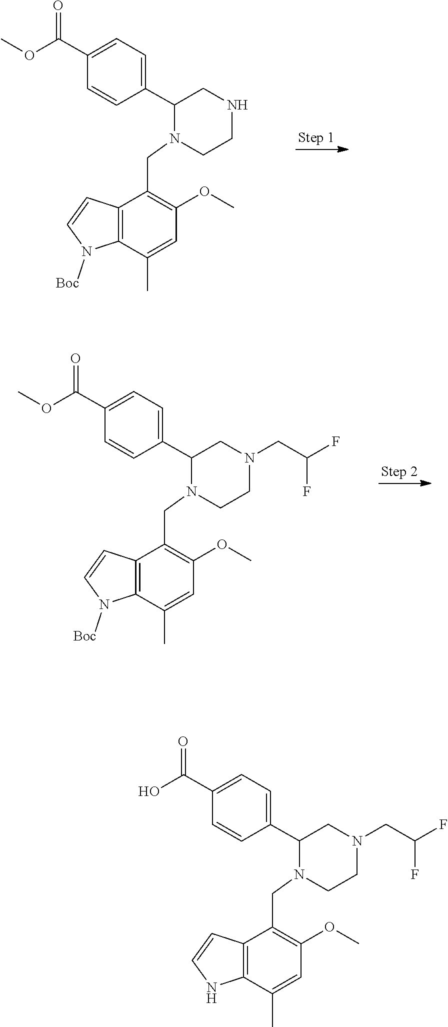

Step 1. tert-Butyl 4-((4-(2,2-difluoroethyl)-2-(4-(methoxycarbonyl)phenyl)piperazin-1-yl)methyl)-5-methoxy-7-methyl-1H-indole-1-carboxylate was prepared with the following procedure. To a solution of tert-butyl 5-methoxy-4-((2-(4-(methoxycarbonyl)phenyl)piperazin-1-yl)methyl)-7-methyl-1H-indole-1-carboxylate (50 mg, 1.0 eq, 0.10 mmol) in THE (1 mL) were added Et₃N (21 μL, 1.5 eq, 0.15 mmol) and 2,2-difluoroethyl trifluoromethanesulphonate (17 μL, 1.25 eq, 0.13 mmol). The resulting mixture was stirred at 20° C. for 15 h. The reaction was worked up; concentrated under reduced pressure gave crude tert-butyl 4-((4-(2,2-difluoroethyl)-2-(4-(methoxycarbonyl)phenyl)piperazin-1-yl)methyl)-5-methoxy-7-methyl-1H-indole-1-carboxylate. (General 2) RT=2.16 min; area % (254 nm)=83%; m/z=558.3 [M+H]⁺.

4-(4-(2,2-Difluoroethyl)-1-((5-methoxy-7-methyl-1H-indol-4-yl)methyl)piperazin-2-yl)benzoic acid was prepared according to General Experimental Procedure 2. Crude tert-butyl 4-((4-(2,2-difluoroethyl)-2-(4-(methoxycarbonyl)phenyl)piperazin-1-yl)methyl)-5-methoxy-7-methyl-1H-indole-1-carboxylate gave 4-(4-(2,2-difluoroethyl)-1-((5-methoxy-7-methyl-1H-indol-4-yl)methyl)piperazin-2-yl)benzoic acid (18 mg, 41% over two steps) as a white solid. LCMS-5 (31697 LCMS-5 C3) RT: 1.830 min; area % (215 nm): 97.2%; m/z=444.2 [M+H]⁺. ¹H-NMR (400 MHz, DMSO-d₆) δ 10.82 (s, 1H), 7.98 (d, J=8.0 Hz, 2H), 7.69 (d, J=7.7 Hz, 2H), 7.26 (t, J=2.9 Hz, 1H), 6.65 (d, J=2.9 Hz, 1H), 6.43 (t, J=2.6 Hz, 1H), 6.11 (tt, J=55.8, 4.3 Hz, 1H), 3.70 (d, J=2.8 Hz, 3H), 3.56 (d, J=11.9 Hz, 1H), 3.42 (dd, J=10.3, 3.0 Hz, 2H), 3.23 (d, J=11.8 Hz, 2H), 2.92-2.58 (m, 6H), 2.42 (s, 3H), 2.32 (q, J=2.1 Hz, 1H), 2.30-2.07 (m, 3H); ¹H-¹⁹F-coupling observed. ¹⁹F-NMR-HDEC (376 MHz, DMSO-d₆) δ −118.70, −118.72.

### Example 33: 4-(1-((5-methoxy-7-methyl-1H-indol-4-yl)methyl)-4-(2-methoxyethyl)piperazin-2-yl)benzoic acid

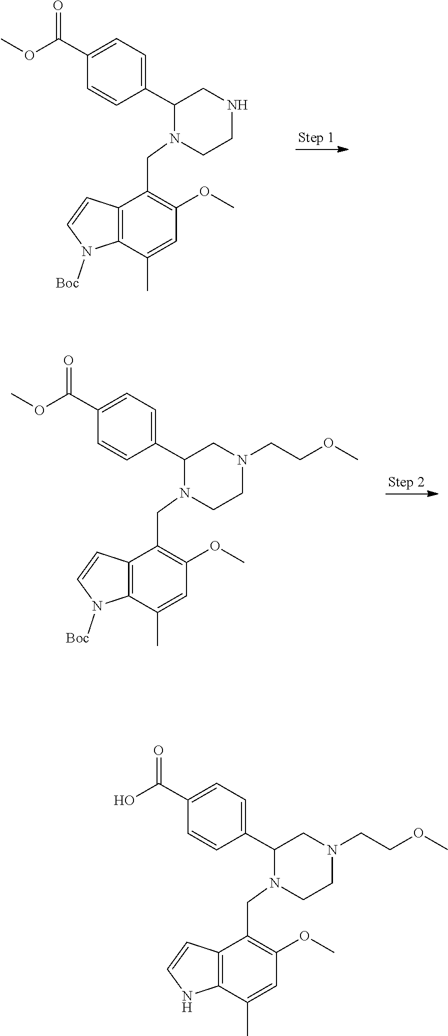

Step 1. tert-Butyl 5-methoxy-4-((2-(4-(methoxycarbonyl)phenyl)-4-(2-methoxyethyl)piperazin-1-yl)methyl)-7-methyl-1H-indole-1-carboxylate was prepared with the following procedure. To a solution of tert-butyl 5-methoxy-4-((2-(4-(methoxycarbonyl)phenyl)piperazin-1-yl)methyl)-7-methyl-1H-indole-1-carboxylate (32 mg, 1.0 eq, 65 μmol) in THE (1 mL) were added Et₃N (14 μL, 1.5 eq, 97 μmol) and 2-bromoethylmethyl ether (7.4 μL, 1.2 eq, 78 μmol). The resulting mixture was heat at 45° C. for 15 h. LCMS indicated incomplete conversion. Additional Et₃N (14 μL, 1.5 Eq, 97 μmol) and 2-bromoethylmethyl ether (7.4 μL, 1.2 eq, 78 μmol) were added and the mixture heated at 45° C. for 15 h. Further, but incomplete, conversion was observed by LCMS. Et₃N (28 μL, 3.0 eq, 0.19 mmol) and 2-bromoethylmethyl ether (14 μL, 2.4 eq, 0.16 mmol) were added and the mixture heated at 50° C. for 15 h. The reaction was worked up; concentrated under reduced pressure gave crude tert-butyl 5-methoxy-4-((2-(4-(methoxycarbonyl)phenyl)-4-(2-methoxyethyl)piperazin-1-yl)methyl)-7-methyl-1H-indole-1-carboxylate. LCMS (General 3) RT=2.01 min; area % (254 nm)=69%; m/z=552.3 [M+H]⁺.

4-(1-((5-Methoxy-7-methyl-1H-indol-4-yl)methyl)-4-(2-methoxyethyl)piperazin-2-yl)benzoic acid was prepared according to General Experimental Procedure 2. Crude tert-butyl 5-methoxy-4-((2-(4-(methoxycarbonyl)phenyl)-4-(2-methoxyethyl)piperazin-1-yl)methyl)-7-methyl-1H-indole-1-carboxylate gave 4-(1-((5-methoxy-7-methyl-1H-indol-4-yl)methyl)-4-(2-methoxyethyl)piperazin-2-yl)benzoic acid (2.1 mg, 7.4% over two steps) as a white solid. LCMS-5 (31697 LCMS-5 C3) RT: 1.531 min; area % (215 nm): 96.0%; m/z=438.2 [M+H]⁺. ¹H-NMR (400 MHz, DMSO-d₆) δ 10.82 (s, 1H), 7.96 (d, J=8.3 Hz, 2H), 7.67 (d, J=7.8 Hz, 2H), 7.25 (t, J=2.8 Hz, 1H), 6.76-6.56 (m, 1H), 6.44 (dd, J=3.1, 1.9 Hz, 1H), 3.70 (s, 3H), 3.56 (d, J=11.8 Hz, 1H), 3.49-3.35 (m, 4H), 3.22 (d, J=11.8 Hz, 1H), 3.18 (s, 3H), 2.85-2.69 (m, 2H), 2.67 (s, 1H), 2.65-2.57 (m, 1H), 2.41 (d, J=2.0 Hz, 5H), 2.33 (p, J=1.9 Hz, OH), 2.20 (td, J=11.6, 2.6 Hz, 1H), 2.10-1.93 (m, 2H).

### Example 34: Enantiomer 1 of 4-(4-cyclopropyl-1-((5-methoxy-7-methyl-1H-indol-4-yl)methyl)piperazin-2-yl)benzoic acid

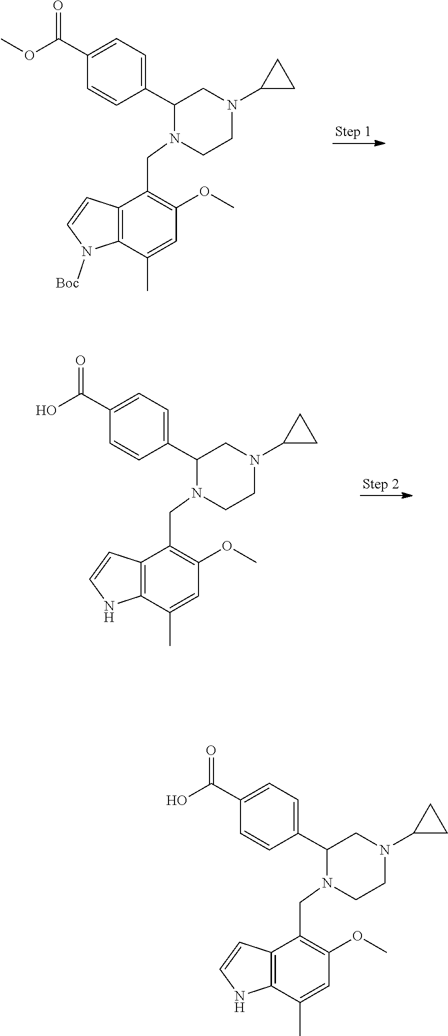 [SMILES: COC(=O)c1ccc(C2CN(C3CC3)CCN2Cc2c(OC)cc(C)c3c2ccn3C(=O)OC(C)(C)C)cc1 ; COc1cc(C)c2[nH]ccc2c1CN1CCN(C2CC2)CC1c1ccc(C(=O)O)cc1 ; COc1cc(C)c2[nH]ccc2c1CN1CCN(C2CC2)CC1c1ccc(C(=O)O)cc1]

Step 1. 4-(4-Cyclopropyl-1-((5-methoxy-7-methyl-1H-indol-4-yl)methyl)piperazin-2-yl)benzoic acid was prepared according General Procedure 2. tert-Butyl 4-((4-cyclopropyl-2-(4-(methoxycarbonyl)phenyl)piperazin-1-yl)methyl)-5-methoxy-7-methyl-1H-indole-1-carboxylate gave 4-(4-cyclopropyl-1-((5-methoxy-7-methyl-1H-indol-4-yl)methyl)piperazin-2-yl)benzoic acid as a white solid (155 mg, 46%). LCMS-5 (31697 LCMS-5 C3) RT: 1.826 min; area % (215 nm): 97.0%; m/z=420.2 [M+H]⁺.

Step 2. Enantiomer 1 of 4-(4-cyclopropyl-1-((5-methoxy-7-methyl-1H-indol-4-yl)methyl)piperazin-2-yl)benzoic acid was prepared with the following procedure. Racemic 4-(4-cyclopropyl-1-((5-methoxy-7-methyl-1H-indol-4-yl)methyl)piperazin-2-yl)benzoic acid was separated in its enantiomers using preparative SFC (31697-1051-04). Enantiomer 1 of 4-(4-cyclopropyl-1-((5-methoxy-7-methyl-1H-indol-4-yl)methyl)piperazin-2-yl)benzoic acid was the first eluting enantiomer and obtained as an off-white solid. LCMS-5 (31697 LCMS-5 C3) RT: 1.828 min; area % (215 nm): 98.1%; m/z=420.2 [M+H]⁺. Chiral SFC (31697F UPC² 10m Cel2 MA1) RT=4.389 min; area % (220 nm): >99%; m/z=420.2.2 [M+H]⁺. ¹H-NMR was identical to that of Example 26.

### Example 35: Enantiomer 2 of 4-(4-Cyclopropyl-1-((5-methoxy-7-methyl-1H-indol-4-yl)methyl)piperazin-2-yl)benzoic acid

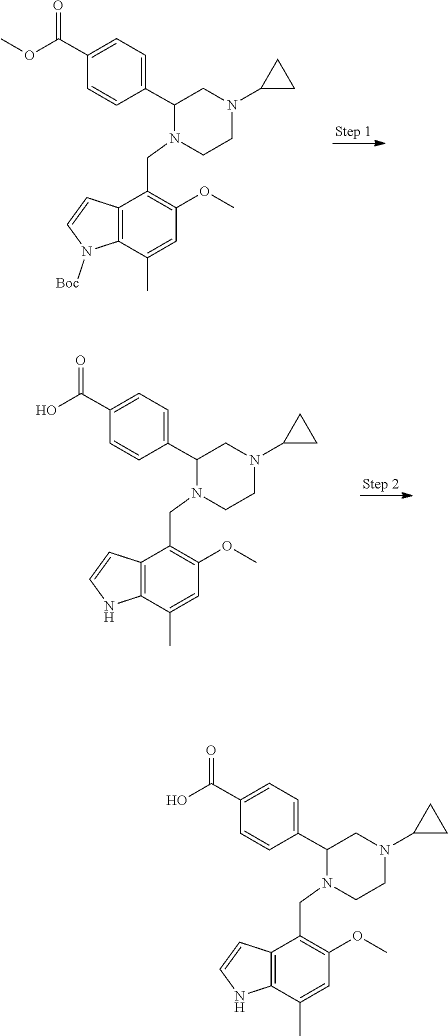 [SMILES: COC(=O)c1ccc(C2CN(C3CC3)CCN2Cc2c(OC)cc(C)c3c2ccn3C(=O)OC(C)(C)C)cc1 ; COc1cc(C)c2[nH]ccc2c1CN1CCN(C2CC2)CC1c1ccc(C(=O)O)cc1 ; COc1cc(C)c2[nH]ccc2c1CN1CCN(C2CC2)CC1c1ccc(C(=O)O)cc1]

Step 1. 4-(4-Cyclopropyl-1-((5-methoxy-7-methyl-1H-indol-4-yl)methyl)piperazin-2-yl)benzoic acid was prepared according General Procedure 2. tert-Butyl 4-((4-cyclopropyl-2-(4-(methoxycarbonyl)phenyl)piperazin-1-yl)methyl)-5-methoxy-7-methyl-1H-indole-1-carboxylate gave 4-(4-cyclopropyl-1-((5-methoxy-7-methyl-1H-indol-4-yl)methyl)piperazin-2-yl)benzoic acid as a white solid (155 mg, 46%). LCMS-5 (31697 LCMS-5 C3) RT: 1.826 min; area % (215 nm): 97.0%; m/z=420.2 [M+H]⁺.

Step 2. Enantiomer 2 of 4-(4-cyclopropyl-1-((5-methoxy-7-methyl-1H-indol-4-yl)methyl)piperazin-2-yl)benzoic acid was prepared with the following procedure. Racemic 4-(4-cyclopropyl-1-((5-methoxy-7-methyl-1H-indol-4-yl)methyl)piperazin-2-yl)benzoic acid was separated in its enantiomers using preparative SFC (31697-1051-04). Enantiomer 2 of 4-(4-cyclopropyl-1-((5-methoxy-7-methyl-1H-indol-4-yl)methyl)piperazin-2-yl)benzoic acid was the second eluting enantiomer and obtained as an off-white solid. LCMS-5 (31697 LCMS-5 C3) RT: 1.829 min; area % (215 nm): 97.8%; m/z=420.2 [M+H]⁺. Chiral SFC (31697F UPC² 10m Cel2 MA1) RT=3.959 min; area % (220 nm): 98.6%; m/z=420.2.2 [M+H]⁺. ¹H-NMR was identical to that of Example 26.

### Example 36: Enantiomer 1 of tert-butyl 5-methoxy-4-((2-(4-(methoxycarbonyl)phenyl)piperazin-1-yl)methyl)-7-methyl-1H-indole-1-carboxylate

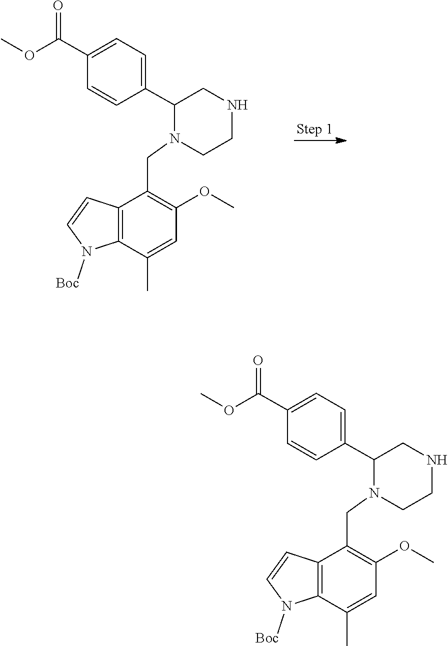

Step 1. Enantiomer 1 of tert-butyl 5-methoxy-4-((2-(4-(methoxycarbonyl)phenyl)piperazin-1-yl)methyl)-7-methyl-1H-indole-1-carboxylate was prepared with the following method. Racemic tert-butyl 5-methoxy-4-((2-(4-(methoxycarbonyl)phenyl)piperazin-1-yl)methyl)-7-methyl-1H-indole-1-carboxylate was separated on preparative chiral SFC (31697 EKN ADH 4-40% 20 min) to obtain enantiomer 1 of tert-butyl 5-methoxy-4-((2-(4-(methoxycarbonyl)phenyl)piperazin-1-yl)methyl)-7-methyl-1H-indole-1-carboxylate. First eluting enantiomer (Amy Grad EE) RT=3.79 min, >99% ee, m/z=494.2 [M+H]⁺. This enantiomer was converted according to Example 4 and gave biological activity similar to Example 24.

### Example 37: Enantiomer 2 of tert-butyl 5-methoxy-4-((2-(4-(methoxycarbonyl)phenyl)piperazin-1-yl)methyl)-7-methyl-1H-indole-1-carboxylate

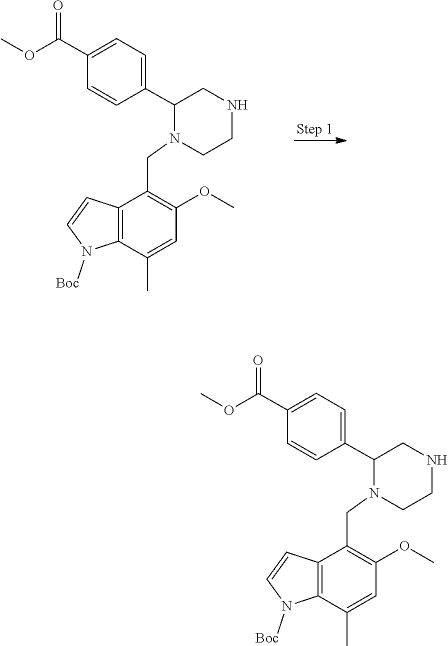

Step 1. Enantiomer 2 of tert-butyl 5-methoxy-4-((2-(4-(methoxycarbonyl)phenyl)piperazin-1-yl)methyl)-7-methyl-1H-indole-1-carboxylate was prepared with the following method. Racemic tert-butyl 5-methoxy-4-((2-(4-(methoxycarbonyl)phenyl)piperazin-1-yl)methyl)-7-methyl-1H-indole-1-carboxylate was separated on preparative chiral SFC (31697 EKN ADH 4-40% 20 min) to obtain enantiomer 2 of tert-butyl 5-methoxy-4-((2-(4-(methoxycarbonyl)phenyl)piperazin-1-yl)methyl)-7-methyl-1H-indole-1-carboxylate. Second eluting enantiomer (Amy Grad EE) RT=5.78 min, >98.2% ee, m/z=494.2 [M+H]⁺. This enantiomer was converted according to Example 4 and gave biological activity similar to Example 25.

### Example 38: Enantiomer of 4-(4-(2-fluoroethyl)-1-((5-methoxy-7-methyl-1H-indol-4-yl)methyl)piperazin-2-yl)benzoic acid

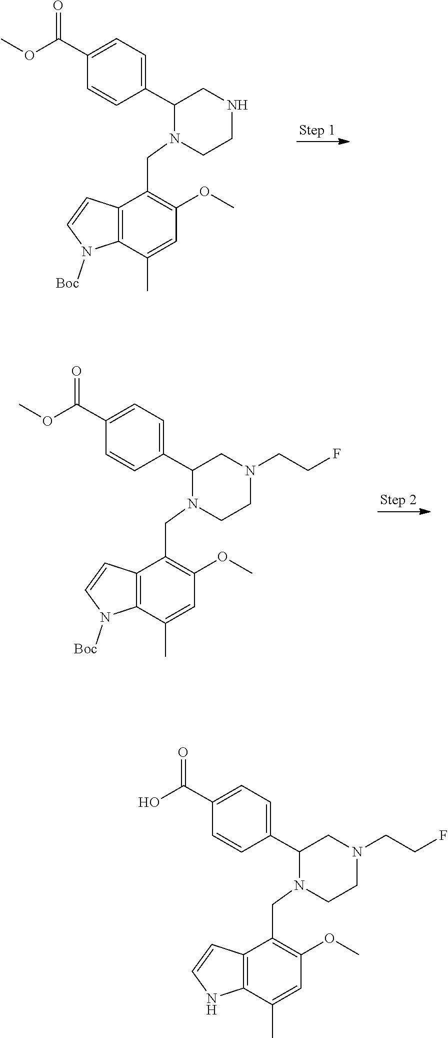

Step 1. Enantiomer of 4-((4-(2-fluoroethyl)-2-(4-(methoxycarbonyl)phenyl)piperazin-1-yl)methyl)-5-methoxy-7-methyl-1H-indole-1-carboxylate was prepared with the following procedure. To a solution of enantiomer 2 of tert-butyl 5-methoxy-4-((2-(4-(methoxycarbonyl)phenyl)piperazin-1-yl)methyl)-7-methyl-1H-indole-1-carboxylate (50 mg, 1.0 eq, 0.10 mmol) in THE (2 mL) were added Et₃N (15 μL, 1.1 eq, 0.11 mmol) and 1-fluoro-2-iodoethane (75 μL, 6.3 eq, 0.64 mmol). The resulting mixture was stirred at 20° C. for 15 h. The reaction was worked up; concentrated under reduced pressure to obtain crude enantiomer of tert-butyl 4-((4-(2-fluoroethyl)-2-(4-(methoxycarbonyl)phenyl)piperazin-1-yl)methyl)-5-methoxy-7-methyl-1H-indole-1-carboxylate. (General3 LCMS-4 AB) RT=2.62 min; area % (254 nm)=43%; m/z=540.4 [M+H]⁺.

Enantiomer of 4-(4-(2-fluoroethyl)-1-((5-methoxy-7-methyl-1H-indol-4-yl)methyl)piperazin-2-yl)benzoic acid was prepared according to General Experimental Procedure 2. Crude enantiomer of tert-butyl 4-((4-(2-fluoroethyl)-2-(4-(methoxycarbonyl)phenyl)piperazin-1-yl)methyl)-5-methoxy-7-methyl-1H-indole-1-carboxylate gave enantiomer of 4-(4-(2-fluoroethyl)-1-((5-methoxy-7-methyl-1H-indol-4-yl)methyl)piperazin-2-yl)benzoic acid (25 mg, 63% over two steps) as a beige solid. LCMS-5 (31697 LCMS-5 C3) RT: 1.576 min; area % (215 nm): 91.0%; m/z=426.2 [M+H]⁺. ¹H-NMR and ¹⁹F-NMR were identical to that of Example 31.

### Example 39: Enantiomer of 4-(4-(2,2-difluoroethyl)-1-((5-methoxy-7-methyl-1H-indol-4-yl)methyl)piperazin-2-yl)benzoic acid

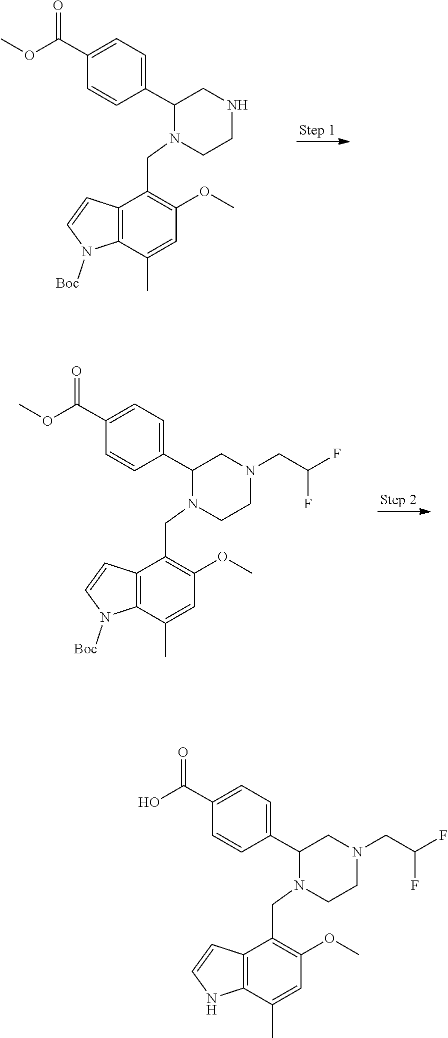

Step 1. Enantiomer of tert-butyl 4-((4-(2,2-difluoroethyl)-2-(4-(methoxycarbonyl)phenyl)piperazin-1-yl)methyl)-5-methoxy-7-methyl-1H-indole-1-carboxylate was prepared with the following procedure. To a solution of enantiomer 2 of tert-butyl 5-methoxy-4-((2-(4-(methoxycarbonyl)phenyl)piperazin-1-yl)methyl)-7-methyl-1H-indole-1-carboxylate (52 mg, 1.0 eq, 0.11 mmol) in THE (1 mL) were added Et₃N (26 μL, 1.8 eq, 0.19 mmol) and 2,2,-difluoroethyl trifluoromethanesulfonate (19 μL, 1.5 eq, 0.16 mmol). The resulting mixture was stirred at 20° C. for 15 h. The reaction was worked up; concentrated under reduced pressure to obtain crude enantiomer of tert-butyl 4-((4-(2,2-difluoroethyl)-2-(4-(methoxycarbonyl)phenyl)piperazin-1-yl)methyl)-5-methoxy-7-methyl-1H-indole-1-carboxylate. (General3 LCMS-4 AB) RT=2.76 min; area % (254 nm)=73%; m/z=558.4 [M+H]⁺.

Step 2. Enantiomer of 4-(4-(2,2-difluoroethyl)-1-((5-methoxy-7-methyl-1H-indol-4-yl)methyl)piperazin-2-yl)benzoic acid was prepared according to General Experimental Procedure 2. Crude enantiomer of tert-butyl 4-((4-(2,2-difluoroethyl)-2-(4-(methoxycarbonyl)phenyl)piperazin-1-yl)methyl)-5-methoxy-7-methyl-1H-indole-1-carboxylate gave enantiomer of 4-(4-(2,2-difluoroethyl)-1-((5-methoxy-7-methyl-1H-indol-4-yl)methyl)piperazin-2-yl)benzoic acid (21 mg, 44% over two steps) as a beige solid. LCMS-5 (Method A) RT: 1.833 min; area % (215 nm): >99%; m/z=444.2 [M+H]⁺. ¹H-NMR and ¹⁹F-NMR was identical to that of Example 32.

### Example 40: (R)-4-(1-((5-Methoxy-7-methyl-1H-indol-4-yl)methyl)-4-methyl-3-oxopiperazin-2-yl)benzoic acid

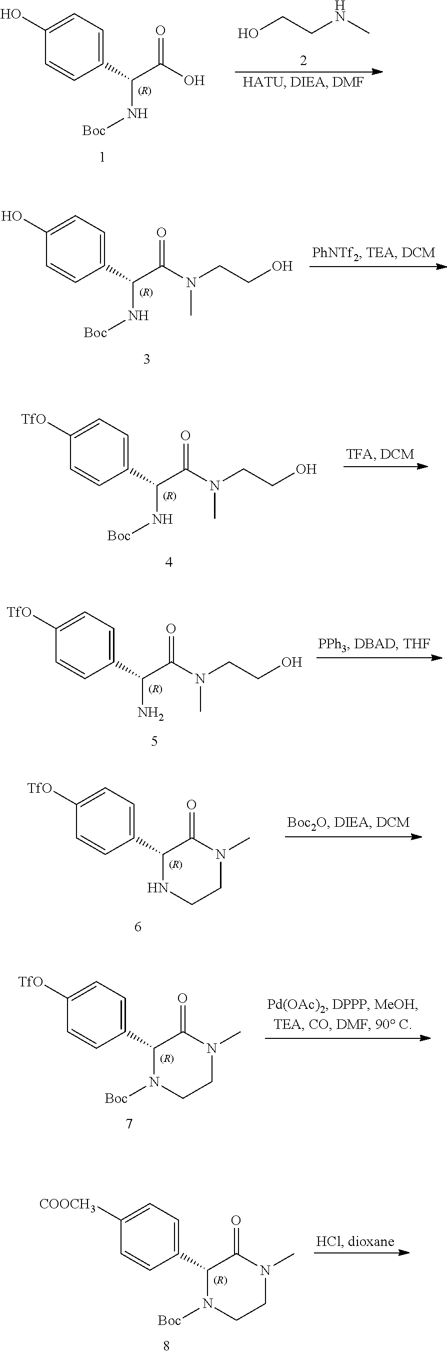

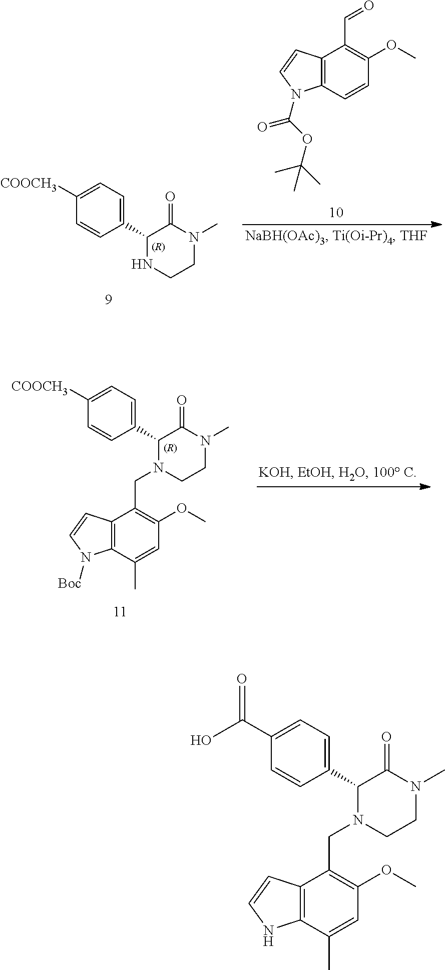

Step 1: tert-Butyl (R)-(2-((2-hydroxyethyl)(methyl)amino)-1-(4-hydroxyphenyl)-2-oxoethyl)carbamate. A solution of (R)-2-((tert-butoxycarbonyl)amino)-2-(4-hydroxyphenyl)acetic acid (1.00 g, 3.74 mmol), methylethanolamine (0.42 g, 5.61 mmol), 2-(7-Azabenzotriazol-1-yl)-N,N,N′,N-tetramethyluronium hexafluorophosphate (2.13 g, 5.61 mmol) and N,N-diisopropylethylamine (1.45 g, 11.22 mmol) in DMF (10 mL) was stirred for 2 h at rt. The resulting mixture was quenched with water (10 mL) and extracted with ethyl acetate (3×10 mL) and the organic layers were combined. The organic layer was washed with brine (10 mL) and dried over anhydrous sodium sulfate. After filtration, the filtrate was concentrated under vacuum. The residue was purified by silica gel column chromatography eluting with PE/EA (0-40%) to afford the title compound (1.00 g, 82.4% yield) as a yellow oil. LCMS (ESI): [M+H]⁺=325.2.

Step 2: (R)-4-(1-((tert-Butoxycarbonyl)amino)-2-((2-hydroxyethyl)(methyl)amino)-2-oxoethyl)phenyl trifluoromethanesulfonate. A solution of tert-butyl (R)-(2-((2-hydroxyethyl)(methyl)amino)-1-(4-hydroxyphenyl)-2-oxoethyl)carbamate (1.00 g, 3.08 mmol), N-phenyl-bis(trifluoromethanesulfonimide) (1.65 g, 4.62 mmol) and triethylamine (0.94 g, 9.24 mmol) in dichloromethane (10 mL) was stirred at rt for 2 h. The resulting mixture was quenched with water (10 mL) and extracted with dichloromethane (3×10 mL) and the organic layers were combined. The organic layer was washed with brine (10 mL) and dried over anhydrous sodium sulfate. After filtration, the filtrate was concentrated under vacuum. The residue was purified by silica gel column chromatography eluting with dichloromethane/methanol (0-10%) to afford the title compound (1.10 g, 78.1% yield) as a yellow oil. LCMS (ESI): [M+H]⁺=457.1.

Step 3: (R)-4-(1-Amino-2-((2-hydroxyethyl)(methyl)amino)-2-oxoethyl)phenyl trifluoromethanesulfonate. A solution of (R)-4-(1-((tert-butoxycarbonyl)amino)-2-((2-hydroxyethyl)(methyl)amino)-2-oxoethyl)phenyl trifluoromethanesulfonate (1.1 g, 2.4 mmol) in dichloromethane (3 mL) was added TFA (3 mL) and stirred for 2 h at rt. The solvent was concentrated under vacuum. The residue was purified by reverse phase chromatography (acetonitrile/10 mM ammonium bicarbonate in water) to afford the title compound (714 mg, 83.2% yield) as a yellow oil. LCMS (ESI): [M+H]⁺=357.1.

Step 4: (R)-4-(4-Methyl-3-oxopiperazin-2-yl)phenyl trifluoromethanesulfonate. A solution of (R)-4-(1-amino-2-((2-hydroxyethyl)(methyl)amino)-2-oxoethyl)phenyl trifluoromethanesulfonate (714 mg, 2.00 mmol) in THE (5 mL) was added di-tert-butyl azodicarboxylate (690 mg, 3.00 mmol) and tributylphosphine (609 mg, 3.00 mmol) and stirred for 2 h at rt. The resulting mixture was quenched with water (10 mL) and extracted with ethyl acetate (3×10 mL) and the organic layers were combined. The organic layer was washed with brine (10 mL) and dried over anhydrous sodium sulfate. After filtration, the filtrate was concentrated under vacuum. The residue was purified by silica gel column chromatography eluting with dichloromethane/methanol (0-10%) to afford the title compound (519 mg, 76.6% yield) as a yellow oil. LCMS (ESI): [M+H]⁺=339.1.

Step 5: tert-Butyl (R)-4-methyl-3-oxo-2-(4-(((trifluoromethyl)sulfonyl)oxy)phenyl) piperazine-1-carboxylate. A solution of (R)-4-(4-methyl-3-oxopiperazin-2-yl)phenyl trifluoromethanesulfonate (519 mg, 1.53 mmol), di-tert-butyl dicarbonate (667 mg, 3.06 mmol) and triethylamine (608 mg, 6.12 mmol) in dichloromethane (5 mL) was stirred for 2 h at rt. The resulting mixture was quenched with water (10 mL) and extracted with dichloromethane (3×10 mL) and the organic layers were combined. The organic layer was washed with brine (10 mL) and dried over anhydrous sodium sulfate. After filtration, the filtrate was concentrated under vacuum. The residue was purified by silica gel column chromatography eluting with dichloromethane/methanol (0-10%) to afford the title compound (517 mg, 77.1% yield) as a yellow oil. LCMS (ESI): [M+H]⁺=439.1.

Step 6: tert-Butyl (R)-2-(4-(methoxycarbonyl)phenyl)-4-methyl-3-oxopiperazine-1-carboxylate. Under carbon monoxide atmosphere, a solution of tert-butyl (R)-4-methyl-3-oxo-2-(4-(((trifluoromethyl)sulfonyl)oxy) phenyl)piperazine-1-carboxylate (400 mg, 0.91 mmol), Palladium (II) acetate (20 mg, 0.09 mmol), 1,3-bis(diphenylphosphino)propane (75 mg, 0.18 mmol) and triethylamine (276 mg, 2.73 mmol) in methanol (3 mL) and DMF (3 mL) was stirred for overnight at 90° C. The solvent was concentrated under vacuum. The residue was purified by silica gel column chromatography eluting with PE/EA (0-70%) to afford the title compound (300 mg, 94.3% yield) as a yellow oil. LCMS (ESI): [M+H]⁺=349.2.

Step 7: Methyl (R)-4-(4-methyl-3-oxopiperazin-2-yl)benzoate. A solution of tert-butyl (R)-2-(4-(methoxycarbonyl)phenyl)-4-methyl-3-oxopiperazine-1-carboxylate (300 mg, 0.86 mmol) in dichloromethane (2 mL) was added TFA (2 mL) and stirred for 2 h at rt. The solvent was concentrated under vacuum to afford the title compound (200 mg, 93.5% yield) as a yellow oil. The crude product was used in the next step without purification. LCMS (ESI): [M+H]⁺=249.1.

Step 8: tert-Butyl (R)-5-methoxy-4-((2-(4-(methoxycarbonyl)phenyl)-4-methyl-3-oxopiperazin-1-yl)methyl)-7-methyl-1H-indole-1-carboxylate. A solution of methyl (R)-4-(4-methyl-3-oxopiperazin-2-yl)benzoate (200 mg, 0.80 mmol) and tert-butyl 4-formyl-5-methoxy-7-methyl-1H-indole-1-carboxylate (466 mg, 1.61 mmol) in THE (3 mL) was added titanium tetraisopropanolate (457 mg, 1.61 mmol) at rt. The resulting mixture was stirred for 3 h at rt. Then sodium triacetoxyborohydride (682 mg, 3.22 mmol) was added. The resulting mixture was stirred for 18 h at rt. The resulting mixture was quenched with water (10 mL) and extracted with ethyl acetate (3×10 mL) and the organic layers were combined. The organic layer was washed with brine (10 mL) and dried over anhydrous sodium sulfate. After filtration, the filtrate was concentrated under vacuum. The residue was purified by silica gel column chromatography eluting with PE/EA (0-50%) to afford the title compound (240 mg, 57.1% yield) as a yellow oil. LCMS (ESI): [M+H]⁺=522.3.

Step 9: (R)-4-(1-((5-Methoxy-7-methyl-1H-indol-4-yl)methyl)-4-methyl-3-oxopiperazin-2-yl)benzoic acid. A solution of (R)-4-(1-((5-methoxy-7-methyl-1H-indol-4-yl)methyl)-4-methyl-3-oxopiperazin-2-yl)benzoic acid (200 mg, 0.38 mmol) in ethanol (3 mL) was added 5 M potassium hydroxide (0.2 mL) and stirred for 2 h at 100° C. The reaction was directly purified by reverse phase chromatography (acetonitrile/10 mM ammonium bicarbonate in water) and the solvent was concentrated under vacuum. The crude product was purified by Prep-HPLC to afford the title compound (39.0 mg, 25.2% yield) as a white solid. LCMS (ESI): [M+H]⁺=408.1. ¹H NMR (300 MHz, DMSO-d₆) δ 10.84 (s, 1H), 7.92 (d, J=7.8 Hz, 2H), 7.48 (d, J=8.0 Hz, 2H), 7.21 (s, 1H), 6.66 (s, 1H), 6.19 (d, J=3.0 Hz, 1H), 4.02 (s, 1H), 3.69 (s, 3H), 3.56 (s, 2H), 3.30 (d, J=3.8 Hz, 2H), 3.21-3.11 (m, 1H), 2.81-2.79 (m, 4H), 2.41 (s, 3H).

### Example 41: (R)-4-(1-((5-Methoxy-7-methyl-1H-indol-4-yl)methyl)-4-methyl-5-oxopiperazin-2-yl)benzoic acid

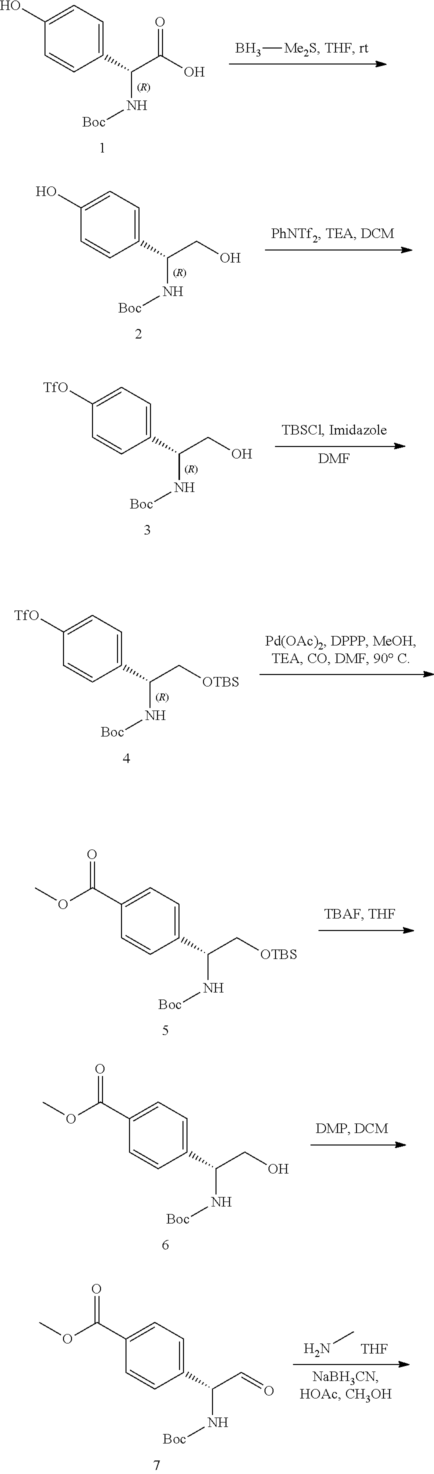

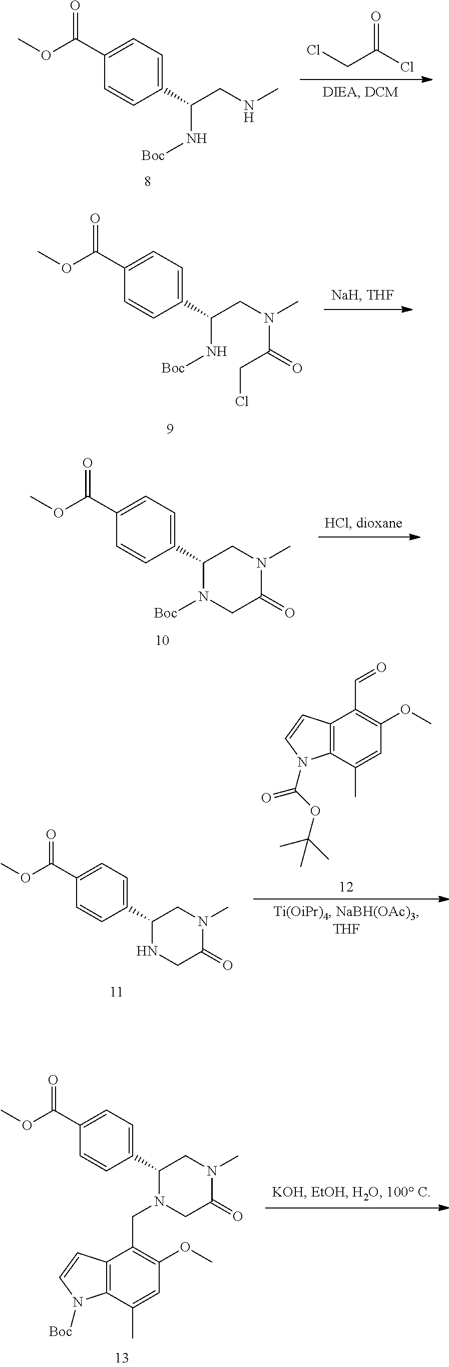

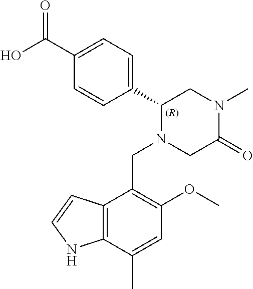

Step 1: tert-Butyl (R)-(2-hydroxy-1-(4-hydroxyphenyl)ethyl)carbamate. To a stirred solution of (R)-2-((tert-butoxycarbonyl)amino)-2-(4-hydroxyphenyl)acetic acid (3.00 g, 11.22 mmol) in THE (30 mL) was added 10 M Borane dimethyl sulfide complex (70 mL) at 0° C. After addition, the reaction was stirred at rt for 5 h. The reaction mixture was quenched with methanol at 0° C. and the solvent was concentrated under vacuum. The residue was purified by silica gel column chromatography eluting with dichloromethane/methanol (0-10%) to afford the title compound (1.10 g, 38.7% yield) as a white solid. LCMS (ESI): [M+H]⁺=254.1.

Step 2: (R)-4-(1-((tert-Butoxycarbonyl)amino)-2-hydroxyethyl)phenyl trifluoromethanesulfonate. A solution of tert-butyl (R)-(2-hydroxy-1-(4-hydroxyphenyl)ethyl)carbamate (1.10 g, 4.34 mmol) in dichloromethane (10 mL) was added triethylamine (1.32 g, 13.02 mmol) and N-phenyl-bis(trifluoromethanesulfonimide) (2.33 g, 6.51 mmol). The resulting solution was stirred at rt for 2 h. The reaction was quenched with water (10 mL) and extracted with dichloromethane (3×10 mL) and the organic layers were combined. The organic layer was washed with brine (10 mL) and dried over anhydrous sodium sulfate. After filtration, the filtrate was concentrated under vacuum. The residue was purified by silica gel column chromatography eluting with dichloromethane/methanol (0-8%) to afford the title compound (1.50 g, 89.6% yield) as a white solid. LCMS (ESI): [M+H]⁺=386.1.

Step 3: (R)-4-(2,2,3,3,10,10-Hexamethyl-8-oxo-4,9-dioxa-7-aza-3-silaundecan-6-yl)phenyl trifluoromethanesulfonate. A solution of (R)-4-(1-((tert-butoxycarbonyl)amino)-2-hydroxyethyl)phenyl trifluoromethanesulfonate (1.40 g, 3.63 mmol), imidazole (0.74 g, 10.89 mmol) and tert-butyldimethylsilyl chloride (0.82 g, 5.45 mmol) in N,N-dimethylformamide (5 mL) was stirred at rt for 2 h. The reaction was quenched with water (10 mL) and extracted with ethyl acetate (3×10 mL). The organic layers were combined. The organic layer was washed with brine (10 mL) and dried over anhydrous sodium sulfate. After filtration, the filtrate was concentrated under vacuum. The residue was purified by silica gel column chromatography eluting with PE/EA (0-60%) to afford the title compound (1.10 g, 60.6% yield) as a yellow oil. LCMS (ESI): [M+H]⁺=500.2.

Step 4: Methyl (R)-4-(2,2,3,3,10,10-hexamethyl-8-oxo-4,9-dioxa-7-aza-3-silaundecan-6-yl)benzoate. Under carbon monoxide atmosphere, a solution of (R)-4-(2,2,3,3,10,10-hexamethyl-8-oxo-4,9-dioxa-7-aza-3-silaundecan-6-yl)phenyl trifluoromethanesulfonate (1.00 g, 2.00 mmol), Palladium (II) Acetate (40 mg, 0.20 mmol), 1,3-Bis(diphenylphosphino)propane (0.17 g, 0.40 mmol) and triethylamine (2.03 g, 20.02 mmol) in methanol (5 mL) and N,N-dimethylformamide (5 mL) was stirred at 90° C. for 12 h. After most of the solvent was removed under vacuum, the residue was purified by reverse phase chromatography (acetonitrile/10 mM ammonium bicarbonate in water) to afford the title compound (700 mg, 85.3% yield) as a yellow oil. LCMS (ESI): [M+H]⁺=410.2.

Step 5: Methyl (R)-4-(1-((tert-butoxycarbonyl)amino)-2-hydroxyethyl)benzoate. A solution of methyl (R)-4-(2,2,3,3,10,10-hexamethyl-8-oxo-4,9-dioxa-7-aza-3-silaundecan-6-yl)benzoate (700 mg, 1.70 mmol) in THE (5 mL) was added 1 M tetrabutylammonium fluoride in THE (2.5 mL, 2.50 mmol) and stirred for 2 h at rt. The reaction was quenched with water (10 mL) and extracted with ethyl acetate (3×10 mL). The organic layers were combined. The organic layer was washed with brine (10 mL) and dried over anhydrous sodium sulfate. After filtration, the filtrate was concentrated under vacuum. The residue was purified by silica gel column chromatography eluting with PE/EA (0-50%) to afford the title compound (460 mg, 91.1% yield) as a yellow oil. LCMS (ESI): [M+H]⁺=296.1.

Step 6: Methyl (R)-4-(1-((tert-butoxycarbonyl)amino)-2-oxoethyl)benzoate. A solution of methyl methyl (R)-4-(1-((tert-butoxycarbonyl)amino)-2-hydroxyethyl)benzoate (460 mg, 1.54 mmol) in dichloromethane (5 mL) was added Dess-Martin periodinane (724 mg, 1.70 mmol) and stirred at rt for 1 h. The reaction was quenched with saturated sodium sulfite (10 mL) and extracted with ethyl acetate (3×10 mL). The organic layers were combined. The organic layer was washed with brine (10 mL) and dried over anhydrous sodium sulfate. After filtration, the filtrate was concentrated under vacuum. The residue was purified by silica gel column chromatography eluting with PE/EA (0-50%) to afford methyl (R)-4-(1-((tert-butoxycarbonyl)amino)-2-oxoethyl)benzoate (350 mg, 35.2%) as a yellow oil. LCMS (ESI): [M+H]⁺=294.1.

Step 7: Methyl (R)-4-(1-((tert-butoxycarbonyl)amino)-2-(methylamino)ethyl)benzoate. A solution of methyl (R)-4-(1-((tert-butoxycarbonyl)amino)-2-oxoethyl)benzoate (350 mg, 1.19 mmol), 2 M methylamine in tetrahydrofuran (1.19 mL, 2.38 mmol) and acetic acid (0.1 mL) in methanol (10 mL) was stirred for 0.5 h at rt. Then sodium cyanoborohydride (149 mg, 2.38 mmol) was added. The resulting solution was stirred for 1 h at rt. The solvent was concentrated under vacuum. The residue was purified by silica gel column chromatography eluting with dichloromethane/methanol (0-10%) to afford the title compound (350 mg, 95.4% yield) as a yellow oil. LCMS (ESI): [M+H]⁺=309.2.

Step 8: Methyl (R)-4-(1-((tert-butoxycarbonyl)amino)-2-(2-chloro-N-methylacetamido)ethyl)benzoate. A solution of methyl (R)-4-(1-((tert-butoxycarbonyl)amino)-2-(methylamino)ethyl)benzoate (350 mg, 1.13 mmol) and N,N-diisopropylethylamine (426 mg, 3.29 mmol) in dichloromethane (5 mL) was added chloroacetyl chloride (248 mg, 2.19 mmol) at 0° C. The resulting mixture was stirred for 0.5 h at 0° C. The reaction was quenched with water (10 mL) and extracted with dichloromethane (3×10 mL). The organic layers were combined. The organic layer was washed with brine (10 mL) and dried over anhydrous sodium sulfate. After filtration, the filtrate was concentrated under vacuum. The residue was purified by silica gel column chromatography eluting with PE/EA (0-50%) to afford the title compound (272 mg, 64.1% yield) as a yellow oil. LCMS (ESI): [M+H]⁺=385.1.

Step 9: tert-Butyl (R)-2-(4-(methoxycarbonyl)phenyl)-4-methyl-5-oxopiperazine-1-carboxylate. To a stirred solution of methyl (R)-4-(1-((tert-butoxycarbonyl)amino)-2-(2-chloro-N-methylacetamido)ethyl)benzoate (272 mg, 0.70 mmol) in THE (5 mL) was added sodium hydride (143 mg, 3.54 mmol) at 0° C. The resulting mixture was stirred for 1 h at rt. The reaction was quenched with saturated ammonium chloride solution (10 mL) and extracted with ethyl acetate (3×10 mL). The organic layers were combined. The organic layer was washed with brine (10 mL) and dried over anhydrous sodium sulfate. After filtration, the filtrate was concentrated under vacuum. The residue was purified by silica gel column chromatography eluting with dichloromethane/methanol (0-10%) to afford the title compound (360 mg, 59.4% yield) as a yellow oil. LCMS (ESI): [M+H]⁺=349.2.

Step 10: Methyl (R)-4-(4-methyl-5-oxopiperazin-2-yl)benzoate. A solution of tert-butyl (R)-2-(4-(methoxycarbonyl)phenyl)-4-methyl-5-oxopiperazine-1-carboxylate (360 mg, 1.03 mmol) in dichloromethane (1 mL) was added TFA (0.5 mL) and stirred for 1 h at rt. The solvent was concentrated under vacuum to afford the title compound (250 mg, 97.6% yield) as a yellow oil. The crude product was directly used in the next step without purification. LCMS (ESI): [M+H]⁺=249.1.

Step 11: tert-Butyl (R)-5-methoxy-4-((2-(4-(methoxycarbonyl)phenyl)-4-methyl-5-oxopiperazin-1-yl)methyl)-7-methyl-1H-indole-1-carboxylate. A solution of methyl (R)-4-(4-methyl-5-oxopiperazin-2-yl)benzoate (250 mg, 1.00 mmol) and tert-butyl 4-formyl-5-methoxy-7-methylindole-1-carboxylate (580 mg, 2.01 mmol) in THE (5 mL) was added titanium tetraisopropanolate (564 mg, 2.01 mmol) and stirred for 3 h at rt. To the above mixture was added sodium triacetoxyborohydride (841 mg, 4.06 mmol) at rt. The reaction was quenched with water (10 mL) and extracted with ethyl acetate (3×10 mL). The organic layers were combined. The organic layer was washed with brine (10 mL) and dried over anhydrous sodium sulfate. After filtration, the filtrate was concentrated under vacuum. The residue was purified by silica gel column chromatography eluting with PE/EA (0-50%) to afford the title compound (130 mg, 27.6% yield) as a yellow oil. LCMS (ESI): [M+H]⁺=522.3.

Step 12: (R)-4-(1-((5-Methoxy-7-methyl-1H-indol-4-yl)methyl)-4-methyl-5-oxopiperazin-2-yl)benzoic acid. A solution of tert-butyl (R)-5-methoxy-4-((2-(4-(methoxycarbonyl)phenyl)-4-methyl-5-oxopiperazin-1-yl)methyl)-7-methyl-1H-indole-1-carboxylate (80 mg, 0.15 mmol) and 5 M potassium hydroxide (0.2 mL, 1 mmol) in ethanol (2 mL) was stirred for 2 h at 100° C. The residue was purified by reverse phase chromatography (acetonitrile/10 mM Ammonium bicarbonate in water) and the solvent was concentrated under vacuum. The crude product was purified by Prep-HPLC to afford the title compound (26.0 mg, 41.92% yield) as a white solid. LCMS (ESI): [M+H]⁺=408.2. ¹H NMR (300 MHz, DMSO-d₆) δ 10.89 (t, J=2.3 Hz, 1H), 8.00 (d, J=7.8 Hz, 2H), 7.65 (d, J=7.9 Hz, 2H), 7.28 (t, J=2.8 Hz, 1H), 6.68 (s, 1H), 6.44 (dd, J=3.1, 1.8 Hz, 1H), 3.81 (dd, J=9.9, 4.2 Hz, 1H), 3.74 (s, 3H), 3.58 (d, J=11.5 Hz, 1H), 3.51-3.40 (m, 1H), 3.33 (dd, J=12.0, 4.3 Hz, 1H), 3.26 (d, J=11.5 Hz, 1H), 3.03-2.82 (m, 2H), 2.79 (s, 3H), 2.43 (s, 3H).

### Example 42: 4-(1-((5-Cyclopropyl-7-methyl-1H-indol-4-yl)methyl)-4-(2,2-difluoroethyl)piperazin-2-yl)benzoic acid

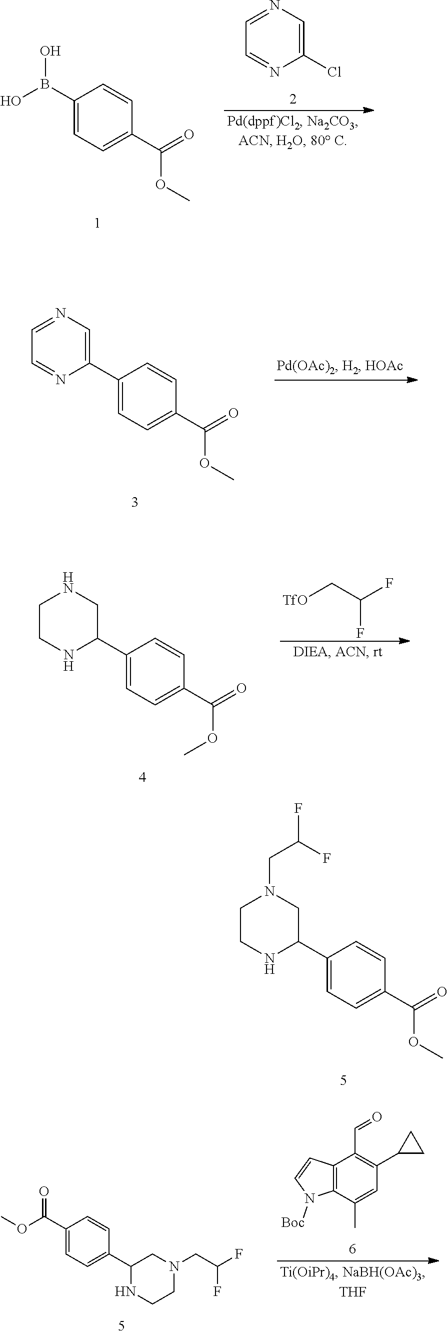

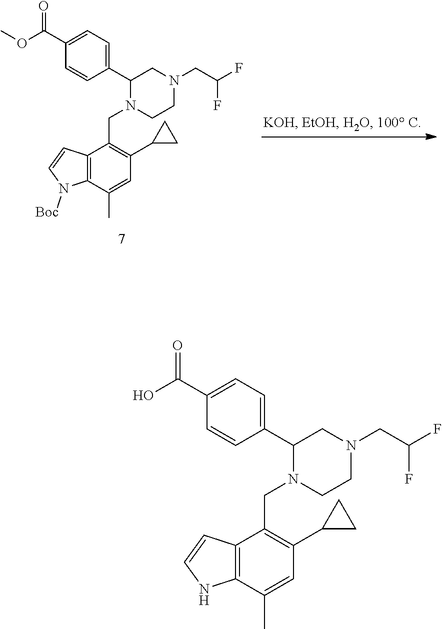 [SMILES: COC(=O)c1ccc(C2CN(CC(F)F)CCN2Cc2c(C3CC3)cc(C)c3c2ccn3C(=O)OC(C)(C)C)cc1 ; Cc1cc(C2CC2)c(CN2CCN(CC(F)F)CC2c2ccc(C(=O)O)cc2)c2cc[nH]c12]

Step 1: Methyl 4-(pyrazin-2-yl)benzoate. Under nitrogen, a mixture of 4-(methoxycarbonyl)phenylboronic acid (100 g, 555 mmol), 2-chloropyrazine (95 g, 833 mmol), 1,1′-bis(diphenylphosphino)ferrocene-palladium(II)dichloride dichloromethane complex (22.63 g, 27.78 mmol), sodium carbonate (177 g, 1.67 mol) in acetonitrile (250 mL) and water (50 mL) was stirred for 2 h at 80° C. The solids were filtered out. The filtrate was concentrated under reduced pressure. The crude product methanol (500 mL) was stirred for overnight and the solids were collected to afford the title compound (70 g, 52.9% yield) as a pink solid. LCMS (ESI): [M+H]⁺=215.3.

Step 2: Methyl 4-(piperazin-2-yl)benzoate. Under hydrogen, a mixture of methyl 4-(pyrazin-2-yl)benzoate (65 g, 303 mmol), palladium (II) acetate (34 g, 151 mmol) in acetic acid (50 mL) was stirred for overnight at rt. The solids were filtered out with celite, concentrated and stripped with toluene (3×100 mL) affording the HOAc salt of the product as off-white solid. The material was dissolved in 1N HCl (500 mL) and then filtered over Celite to remove a dark precipitate. The aqueous layer was washed with ethyl acetate (2×400 mL). The aqueous layer was cooled by the addition of ice and basified with conc. sodium hydroxide to pH=10. The product was extracted with ethyl acetate (2×500 mL). The combined organic layers were washed with brine (100 mL), dried over sodium sulfate and concentrated to afford the title compound (50.2 g, 75.11% yield) as a yellow solid. LCMS (ESI): [M+H]⁺=220.9.

Step 3: Methyl 4-(4-(2,2-difluoroethyl)piperazin-2-yl)benzoate. A solution of methyl 4-(piperazin-2-yl)benzoate (30 g, 136 mmol) in acetonitrile (500 mL) was added N,N-diisopropylethylamine (95 mL, 544 mmol) and 2,2-difluoroethyl trifluoromethanesulfonate (29 g, 136 mmol) and stirred for 12 h at rt. The reaction was quenched with water (500 mL). The resulting mixture was extracted with ethyl acetate (3×500 mL) and the organic layers were combined. The organic layer was washed with brine (2×500 mL), dried over sodium sulfate and concentrated. The solvent was concentrated under vacuum. The residue was purified by silica gel column chromatography eluting with PE/EA Dichloromethane/ethyl acetate (0-50%) to afford the title compound (13 g, 33.57% yield) as a yellow solid. LCMS (ESI): [M+H]⁺=285.3.

Step 4: tert-Butyl 5-cyclopropyl-4-((4-(2,2-difluoroethyl)-2-(4-(methoxycarbonyl)phenyl)piperazin-1-yl)methyl)-7-methyl-1H-indole-1-carboxylate. A solution of methyl 4-(4-(2,2-difluoroethyl)piperazin-2-yl)benzoate (50 mg, 0.17 mmol), tert-butyl 5-cyclopropyl-4-formyl-7-methyl-1H-indole-1-carboxylate (105 mg, 0.35 mmol), titanium tetraisopropanolate (99 mg, 0.35 mmol) in THE (3 mL) was stirred for 3 h at rt. Then sodium triacetoxyborohydride (149 mg, 0.70 mmol) was added at rt. The resulting mixture was stirred for additional 12 h at rt. The reaction was quenched with water (10 mL). The resulting mixture was extracted with ethyl acetate (3×10 mL) and the organic layers were combined. The solvent was concentrated under vacuum. The residue was purified by silica gel column chromatography eluting with PE/EA (0-30%) to afford the title compound (40 mg, 39.1% yield) as a yellow oil. LCMS (ESI): [M+H]⁺=568.3.

Step 5: 4-(1-((5-Cyclopropyl-7-methyl-1H-indol-4-yl)methyl)-4-(2,2-difluoroethyl)piperazin-2-yl)benzoic acid. A solution of tert-butyl 5-cyclopropyl-4-((4-(2,2-difluoroethyl)-2-(4-(methoxycarbonyl)phenyl)piperazin-1-yl)methyl)-7-methyl-1H-indole-1-carboxylate (40 mg, 0.07 mmol) and 5 M potassium hydroxide solution (0.1 mL) in ethanol (1 mL) was stirred for 2 h at 100° C. The residue was directly purified by reverse phase chromatography (acetonitrile/10 mM ammonium bicarbonate in water) and the solvent was concentrated under vacuum. The crude product was purified by Prep-HPLC to afford the title compound (15.0 mg, 48.2% yield) as a white solid. LCMS (ESI): [M+H]⁺=454.2. ¹H NMR (300 MHz, DMSO-d₆) δ 12.68 (brs, 1H), 10.86 (s, 1H), 8.00-7.91 (m, 2H), 7.66 (d, J=7.9 Hz, 2H), 7.24 (t, J=2.8 Hz, 1H), 6.54 (dd, J=3.1, 1.8 Hz, 1H), 6.46 (s, 1H), 6.11 (tt, J=55.7, 4.3 Hz, 1H), 3.74 (d, J=11.9 Hz, 1H), 3.45 (d, J=9.9 Hz, 1H), 3.34 (d, J=5.7 Hz, 2H), 2.90-2.61 (m, 4H), 2.43-2.29 (m, 4H), 2.27-2.04 (m, 3H), 0.90-0.75 (m, 1H), 0.75-0.62 (m, 1H), 0.61-0.43 (m, 1H), 0.23-0.08 (m, 1H).

Among other things, the present disclosure provides compounds and compositions that can modulate complement activation. In some embodiments, provided technologies inhibit complement activation. In some embodiments, provided technologies inhibit C3 convertase. Various technologies are available for assessing provided compounds, compositions and methods. In some embodiments, a useful assay is an assay described below. In some

### Example 43: (S)-4-(1-((5-Cyclopropyl-7-methyl-1H-indol-4-yl)methyl)-4-(2,2-difluoroethyl)piperazin-2-yl)benzoic acid

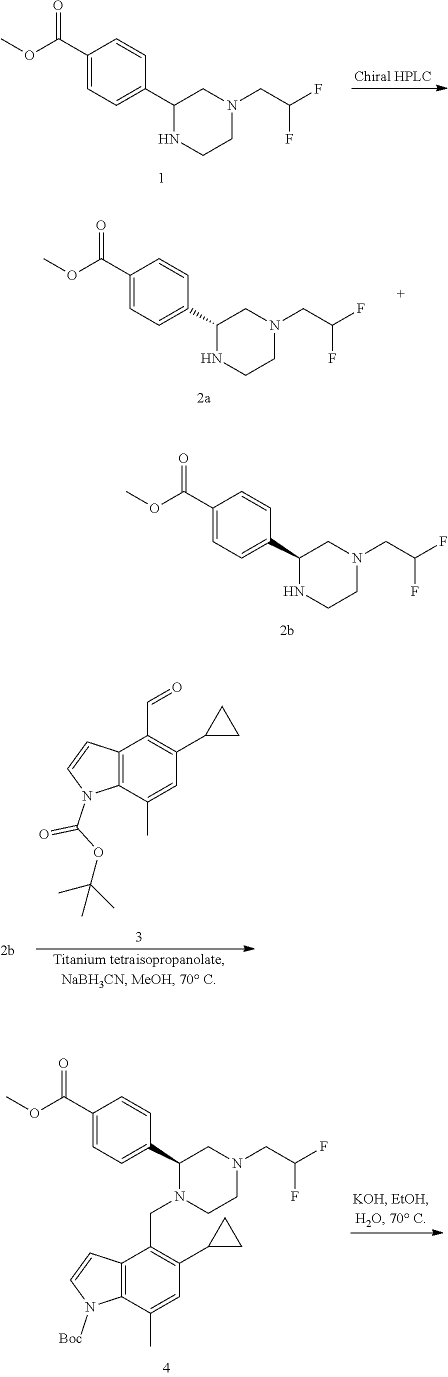 [SMILES: COC(=O)c1ccc(C2CN(CC(F)F)CCN2)cc1 ; COC(=O)c1ccc([C@@H]2CN(CC(F)F)CCN2)cc1 ; COC(=O)c1ccc([C@H]2CN(CC(F)F)CCN2)cc1 ; Cc1cc(C2CC2)c(C=O)c2ccn(C(=O)OC(C)(C)C)c12 ; COC(=O)c1ccc([C@H]2CN(CC(F)F)CCN2Cc2c(C3CC3)cc(C)c3c2ccn3C(=O)OC(C)(C)C)cc1]

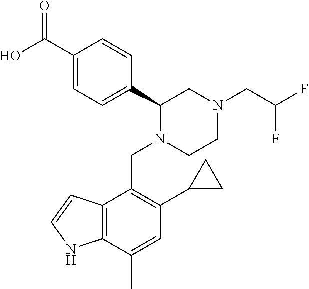 [SMILES: Cc1cc(C2CC2)c(CN2CCN(CC(F)F)C[C@@H]2c2ccc(C(=O)O)cc2)c2cc[nH]c12]

Step 1: Methyl (R)-4-(4-(2,2-difluoroethyl)piperazin-2-yl)benzoate & methyl (S)-4-(4-(2,2-difluoroethyl)piperazin-2-yl)benzoate. Methyl 4-(4-(2,2-difluoroethyl)piperazin-2-yl)benzoate (8 g, 28.17 mmol) was applied for further isolation by Chiral SFC to afford methyl (R)-4-(4-(2,2-difluoroethyl)piperazin-2-yl)benzoate & methyl (S)-4-(4-(2,2-difluoroethyl)piperazin-2-yl)benzoate. Column: CHIRALPAK IF, 7*25 cm, 10 m; Mobile Phase A: C02, Mobile Phase B: MEOH(0.1% 2M NH3-MEOH); Flow rate: 250 mL/min; Gradient: isocratic 40% B; Column Temperature(° C.): 35; Back Pressure(bar): 100; Wave Length: 220 nm; RT1(min): 6.12; RT2(min): 7.21; Sample Solvent: MeOH-Preparative; Injection Volume: 1 mL; Number of Runs: 100. Methyl (R)-4-(4-(2,2-difluoroethyl)piperazin-2-yl)benzoate (3.1 g, 38.8%) as a yellow oil; LCMS (ESI): [M+H]⁺=285.1; ¹H NMR (300 MHz, DMSO-d₆) δ 7.90 (d, J=8.3 Hz, 1H), 7.54 (d, J=8.2 Hz, 1H), 6.15 (tt, J=55.7, 4.3 Hz, 1H), 3.84 (s, 2H), 3.80 (dd, J=10.1, 2.8 Hz, 1H), 2.99-2.63 (m, 3H), 2.35-2.20 (m, 1H), 2.03 (t, J=10.4 Hz, 1H). t_{R}=0.845 min (CHIRALPAK IF-3 3.0*50 mm, 3 μm, SFC CO-solvent MeOH (0.1% DEA), 2.0 mL/min, 220 nm). Methyl (S)-4-(4-(2,2-difluoroethyl)piperazin-2-yl)benzoate (560 mg, 27.5%) as a yellow oil. LCMS (ESI): [M+H]⁺=285.1; ¹H NMR (300 MHz, DMSO-d₆) δ 7.90 (d, J=8.3 Hz, 1H), 7.54 (d, J=8.2 Hz, 1H), 6.15 (tt, J=55.7, 4.3 Hz, 1H), 3.84 (s, 2H), 3.80 (dd, J=10.1, 2.8 Hz, 1H), 2.99-2.63 (m, 3H), 2.35-2.20 (m, 1H), 2.03 (t, J=10.4 Hz, 1H). t_{R}=0.981 min (CHIRALPAK IF-3 3.0*50 mm, 3 μm, SFC CO-solvent MeOH (0.1% DEA), 2.0 mL/min, 220 nm).

Step 2: tert-Butyl 5-cyclopropyl-4-(((2S)-4-(2,2-difluoroethyl)-2-(4-(methoxycarbonyl)phenyl)piperazin-1-yl)methyl)-7-methylindole-1-carboxylate. To a stirred solution of tert-butyl 5-cyclopropyl-4-formyl-7-methylindole-1-carboxylate (100 mg, 0.33 mmol) and methyl 4-((2S)-4-(2,2-difluoroethyl)piperazin-2-yl)benzoate (100 mg, 0.35 mmol) in methanol (4 mL) was added titanium tetraisopropanolate (0.8 mL) dropwise at rt. The resulting mixture was stirred for 8 h at 70° C. To the above mixture was added sodium cyanoborohydride (60 mg, 0.96 mmol) in portion at rt. The resulting mixture was stirred for overnight at rt. The reaction mixture was diluted with water (10 mL). The resulting mixture was extracted with ethyl acetate (3×10 mL) and the organic layers were concentrated under reduced pressure. The residue was purified by silica gel column chromatography, eluted with PE/EA (5:1) to afford the title compound (40 mg, 21.1%) as a white solid. LCMS (ESI): [M+H]⁺=568.2.

Step 3: (S)-4-(1-((5-Cyclopropyl-7-methyl-1H-indol-4-yl)methyl)-4-(2,2-difluoroethyl)piperazin-2-yl)benzoic acid. A mixture of tert-butyl 4-(((2S)-4-(2,2-difluoroethyl)-2-(4-(methoxycarbonyl)phenyl)piperazin-1-yl)methyl)-5-methoxy-7-methylindole-1-carboxylate (40 mg, 0.07 mmol) and potassium hydroxide (40 mg, 0.72 mmol) in water (0.25 mL) and ethanol (5 mL) was stirred for 1 h at 70° C. The reaction mixture was acidified to pH=8 with citric acid. The solvent was removed under vacuum. The residue was purified by reverse phase chromatography (acetonitrile 10%-30%/10 mM ammonium bicarbonate in water) to afford crude product. The crude product was further purified by Prep-HPLC to afford the title compound (11.0 mg, 34.6% yield) as a white solid. LCMS (ESI): [M+H]⁺=454.2; ¹H NMR (300 MHz, DMSO-d₆) δ 12.94 (brs, 1H), 10.84 (s, 1H), 7.94 (d, J=8.1 Hz, 2H), 7.64 (d, J=7.8 Hz, 2H), 7.23 (t, J=2.8 Hz, 1H), 6.56-6.51 (m, 1H), 6.45 (s, 1H), 6.33-5.88 (m, 1H), 3.73 (d, J=11.8 Hz, 1H), 3.48-3.41 (m, 1H), 3.39-3.33 (m, 1H), 2.85-2.66 (m, 4H), 2.56-2.51 (m, 1H), 2.40-2.29 (m, 4H), 2.28-2.17 (m, 2H), 2.16-2.08 (m, 1H), 0.87-0.76 (m, 1H), 0.73-0.63 (m, 1H), 0.56-0.45 (m, 1H), 0.19-0.09 (m, 1H).

### Example 44: 4-(1-((7-Chloro-5-methoxy-1H-indol-4-yl)methyl)-4-(2,2-difluoroethyl)piperazin-2-yl)benzoic acid

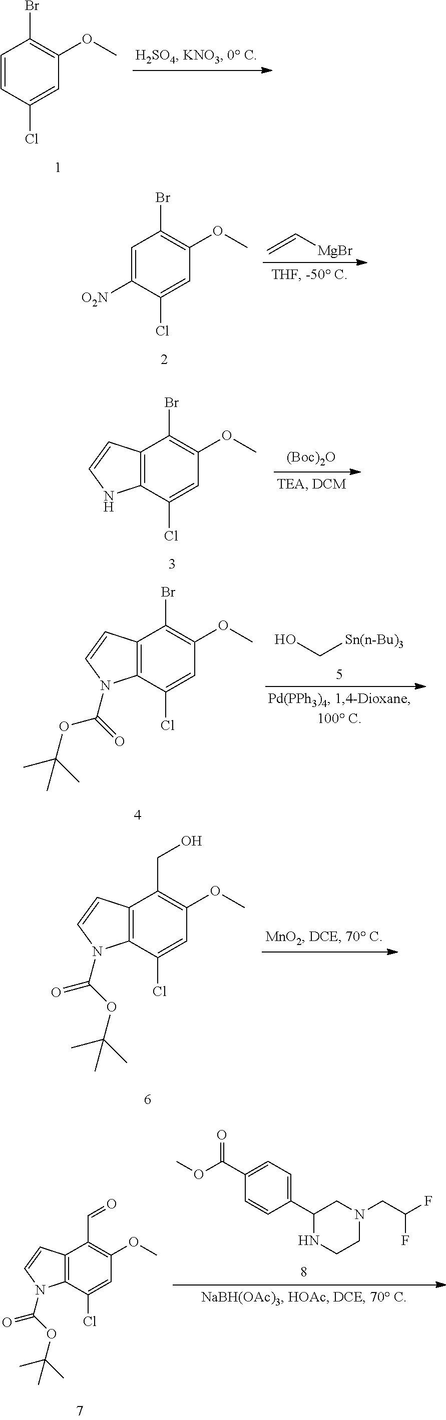

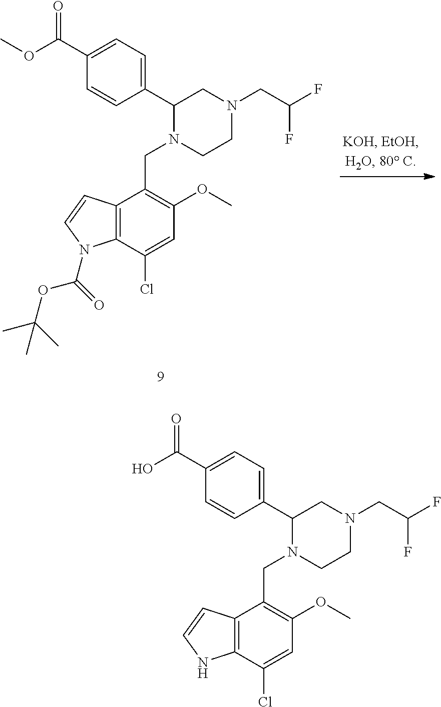 [SMILES: COC(=O)c1ccc(C2CN(CC(F)F)CCN2Cc2c(OC)cc(Cl)c3c2ccn3C(=O)OC(C)(C)C)cc1 ; COc1cc(Cl)c2[nH]ccc2c1CN1CCN(CC(F)F)CC1c1ccc(C(=O)O)cc1]

Step 1: 1-Bromo-4-chloro-2-methoxy-5-nitrobenzene. To a mixture of 1-bromo-4-chloro-2-methoxybenzene (25.3 g, 114.2 mmol) in con. sulfuric acid (120 mL) was added potassium nitrate (8.1 g, 80.0 mmol) in portions and stirred for 1 h at 0° C. The reaction was quenched with ice water. The precipitated solids were collected by filtration and washed with water. The residue was purified by silica gel column chromatography eluting with EA/PE (1:5) to afford the title compound (9.2 g, 30.2% yield) as a white solid. ¹H NMR (300 MHz, Chloroform-d) δ 8.28 (s, 1H), 6.99 (s, 1H), 4.00 (s, 3H).

Step 2: 4-bromo-7-chloro-5-methoxy-1H-indole. To a mixture of 1-bromo-4-chloro-2-methoxy-5-nitrobenzene (9.2 g, 34.40 mmol) in THF (20 mL) was added 1 M bromo(ethenyl)magnesium in THF (166 mL, 166 mmol) at −50° C. under nitrogen atmosphere and stirred at the same temperature for 2 h. The reaction was quenched with saturated ammonium chloride at 0° C. The aqueous layer was extracted with ethyl acetate. The combined organic layers were dried over anhydrous sodium sulfate. After filtration, the filtrate was concentrated under reduced pressure. The residue was purified by silica gel column chromatography eluting with EA/PE (1:5) to afford the title compound (2.5 g, 27.2% yield) as a yellow solid. LCMS (ESI): [M+H]⁺=260.1.

Step 3: tert-Butyl 4-bromo-7-chloro-5-methoxyindole-1-carboxylate. To a mixture of 4-bromo-7-chloro-5-methoxy-1H-indole (2.5 g, 9.60 mmol) in dichloromethane (50 mL) was added triethylamine (4.8 g, 47.43 mmol) and di-tert-butyl dicarbonate (5.3 g, 24.28 mmol) at rt. The reaction mixture was stirred at rt for 1 h. The mixture was diluted with dichloromethane and washed with brine. The combined organic layers were dried over anhydrous sodium sulfate. After filtration, the filtrate was concentrated under reduced pressure. The residue was purified by silica gel column chromatography eluting with EA/PE (1:10) to afford the title compound (3.1 g, 89.5% yield) as a yellow solid. LCMS (ESI): [M+H]⁺=360.1.

Step 4: tert-Butyl 7-chloro-4-(hydroxymethyl)-5-methoxyindole-1-carboxylate. A mixture of tert-butyl 4-bromo-7-chloro-5-methoxyindole-1-carboxylate (662 mg, 1.84 mmol), (tributylstannyl)methanol (897 mg, 2.8 mmol) and tetrakis(triphenylphosphine)palladium (440 mg, 0.38 mmol) in 1,4-dioxane (7 mL) was stirred for 16 h at 100° C. under nitrogen atmosphere. The reaction was quenched with saturated potassium fluoride at rt. The mixture was extracted with dichloromethane and washed with brine. The combined organic layers were dried over anhydrous sodium sulfate. After filtration, the filtrate was concentrated under reduced pressure. The residue was purified by silica gel column chromatography, eluting with EA/PE (0-25%) to afford the title compound (500 mg, 87.4% yield) as a white solid. ¹H NMR (300 MHz, Chloroform-d) δ 7.56 (d, J=3.8 Hz, 1H), 6.97 (s, 1H), 6.65 (d, J=3.8 Hz, 1H), 4.88 (s, 2H), 3.90 (s, 3H), 2.26 (s, 1H), 1.64 (s, 9H).

Step 5: tert-Butyl 7-chloro-4-formyl-5-methoxyindole-1-carboxylate. To a mixture of tert-butyl 7-chloro-4-(hydroxymethyl)-5-methoxyindole-1-carboxylate (500 mg, 1.60 mmol) and manganese dioxide (1.4 g, 16.10 mmol) in 1,2-dichloroethane (10 mL) was stirred for 1 h at 70° C. The resulting mixture was filtered, the filter cake was washed with dichloromethane. The filtrate was concentrated under reduced pressure to afford the title compound (400 mg, 80.6% yield) as a yellow solid. LCMS (ESI): [M+H]⁺=310.0.

Step 6: tert-Butyl 7-chloro-4-((4-(2,2-difluoroethyl)-2-(4-(methoxycarbonyl)phenyl)piperazin-1-yl)methyl)-5-methoxyindole-1-carboxylate. A solution of tert-butyl 7-chloro-4-formyl-5-methoxyindole-1-carboxylate (50 mg, 0.16 mmol), methyl 4-(4-(2,2-difluoroethyl)piperazin-2-yl)benzoate (52 mg, 0.18 mmol) and acetic acid (0.1 mL) in 1,2-dichloroethane (5 mL) was stirred for 1 day at 70° C. The mixture was allowed to cool down to rt. Then sodium triacetoxyborohydride (100 mg, 0.47 mmol) was added and stirred at 70° C. for 2 h. The reaction was quenched with water. The resulting mixture was extracted with dichloromethane. The combined organic layers were washed with brine, dried over anhydrous sodium sulfate. After filtration, the filtrate was concentrated under reduced pressure. The residue was purified by silica gel column chromatography eluting with EA/PE (1:3) to afford the title compound (41 mg, 43.9% yield) as a colorless solid. LCMS (ESI): [M+H]⁺=578.1.

Step 7: 4-(1-((7-Chloro-5-methoxy-1H-indol-4-yl)methyl)-4-(2,2-difluoroethyl)piperazin-2-yl)benzoic acid. To a mixture of tert-butyl 7-chloro-4-((4-(2,2-difluoroethyl)-2-(4-(methoxycarbonyl)phenyl)piperazin-1-yl)methyl)-5-methoxyindole-1-carboxylate (40 mg, 0.07 mmol) and potassium hydroxide (40 mg, 0.71 mmol) in water (0.5 mL) and ethanol (2 mL) was stirred for 30 min at 80° C. The pH was adjusted to 8 with conc. citric acid. The resulting residue was directly purified by reverse phase chromatography (acetonitrile 5%-35%/10 mM NH₄HCO₃ in water) to afford the title compound (17.5 mg, 54.5% yield) as a white solid. LCMS (ESI): [M+H]⁺=464.1; ¹H NMR (300 MHz, DMSO-d₆) δ 12.72 (brs, 1H), 11.21 (d, J=2.4 Hz, 1H), 8.07-7.89 (m, 2H), 7.68 (d, J=7.8 Hz, 2H), 7.36 (t, J=2.8 Hz, 1H), 6.95 (s, 1H), 6.61-6.51 (m, 1H), 6.35-5.88 (m, 1H), 3.74 (s, 3H), 3.56 (d, J=12.0 Hz, 1H), 3.49-3.40 (m, 1H), 3.27 (d, J=12.1 Hz, 1H), 2.89-2.55 (m, 5H), 2.35-2.15 (m, 3H).

### Example 45: (R)-4-(1-((5-Cyclopropyl-7-methyl-1H-indol-4-yl)methyl)-4-(2,2-difluoroethyl)piperazin-2-yl)benzoic acid

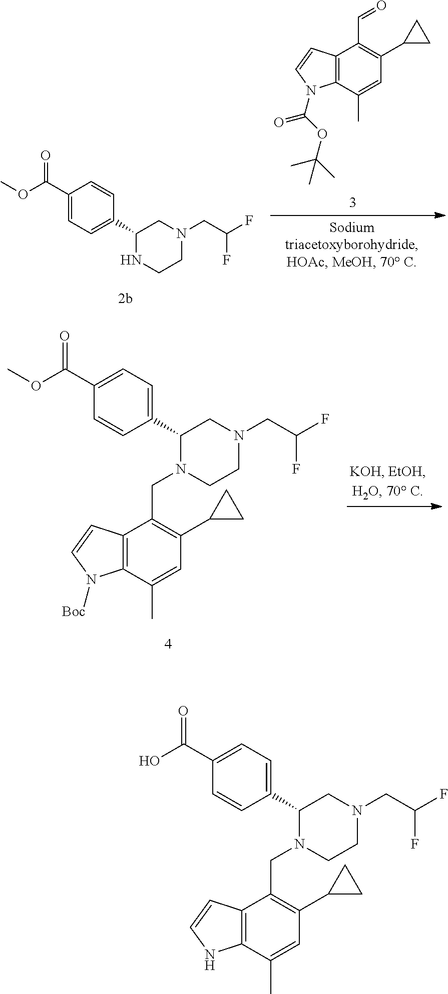 [SMILES: COC(=O)c1ccc([C@@H]2CN(CC(F)F)CCN2)cc1 ; Cc1cc(C2CC2)c(C=O)c2ccn(C(=O)OC(C)(C)C)c12 ; COC(=O)c1ccc([C@@H]2CN(CC(F)F)CCN2Cc2c(C3CC3)cc(C)c3c2ccn3C(=O)OC(C)(C)C)cc1 ; Cc1cc(C2CC2)c(CN2CCN(CC(F)F)C[C@H]2c2ccc(C(=O)O)cc2)c2cc[nH]c12]

Step 1: tert-Butyl 5-cyclopropyl-4-(((2R)-4-(2,2-difluoroethyl)-2-(4-(methoxycarbonyl)phenyl)piperazin-1-yl)methyl)-7-methylindole-1-carboxylate. To a stirred solution of tert-butyl 5-cyclopropyl-4-formyl-7-methylindole-1-carboxylate (50 mg, 0.17 mmol) in methanol (1 mL) was added methyl 4-((2R)-4-(2,2-difluoroethyl)piperazin-2-yl)benzoate (50 mg, 0.18 mmol), sodium triacetoxyborohydride (71 mg, 0.34 mmol) and AcOH (10 mg, 0.17 mmol) at rt. The resulting mixture was stirred for 8 h at 70° C. The solvent was removed under vacuum. The residue was purified by silica gel column chromatography eluting with PE/EA (5:1) to afford the title compound (40 mg, 21.1% yield) as a yellow oil. LCMS (ESI): [M+H]⁺=568.2.

Step 2: (R)-4-(1-((5-Cyclopropyl-7-methyl-1H-indol-4-yl)methyl)-4-(2,2-difluoroethyl)piperazin-2-yl)benzoic acid. A mixture of tert-butyl 4-(((2S)-4-(2,2-difluoroethyl)-2-(4-(methoxycarbonyl)phenyl)piperazin-1-yl)methyl)-5-methoxy-7-methylindole-1-carboxylate (40 mg, 0.07 mmol) and potassium hydroxide (40 mg, 0.72 mmol) in water (0.25 mL) and ethanol (5 mL) was stirred for 1 h at 70° C. The reaction mixture was acidified to pH=8 with citric acid. The solvent was removed under vacuum. The residue was purified by reverse phase chromatography (acetonitrile 10%-30%/10 mM ammonium bicarbonate in water) to afford crude product. The crude product was further purified by Prep-HPLC to afford the title compound (16.8 mg, 52.8%) as a white solid. LCMS (ESI): [M+H]⁺=454.2; ¹H NMR (300 MHz, DMSO-d₆) δ 12.94 (brs, 1H), 10.84 (s, 1H), 7.94 (d, J=8.1 Hz, 2H), 7.64 (d, J=7.8 Hz, 2H), 7.23 (t, J=2.8 Hz, 1H), 6.56-6.51 (m, 1H), 6.45 (s, 1H), 6.33-5.88 (m, 1H), 3.73 (d, J=11.8 Hz, 1H), 3.48-3.41 (m, 1H), 3.39-3.33 (m, 1H), 2.85-2.66 (m, 4H), 2.56-2.51 (m, 1H), 2.40-2.29 (m, 4H), 2.28-2.17 (m, 2H), 2.16-2.08 (m, 1H), 0.87-0.76 (m, 1H), 0.73-0.63 (m, 1H), 0.56-0.45 (m, 1H), 0.19-0.09 (m, 1H).

### Example 46: Enantiomers of 4-(4-((1H-Imidazol-2-yl)methyl)-1-((5-methoxy-7-methyl-1H-indol-4-yl)methyl)piperazin-2-yl)benzoic acid

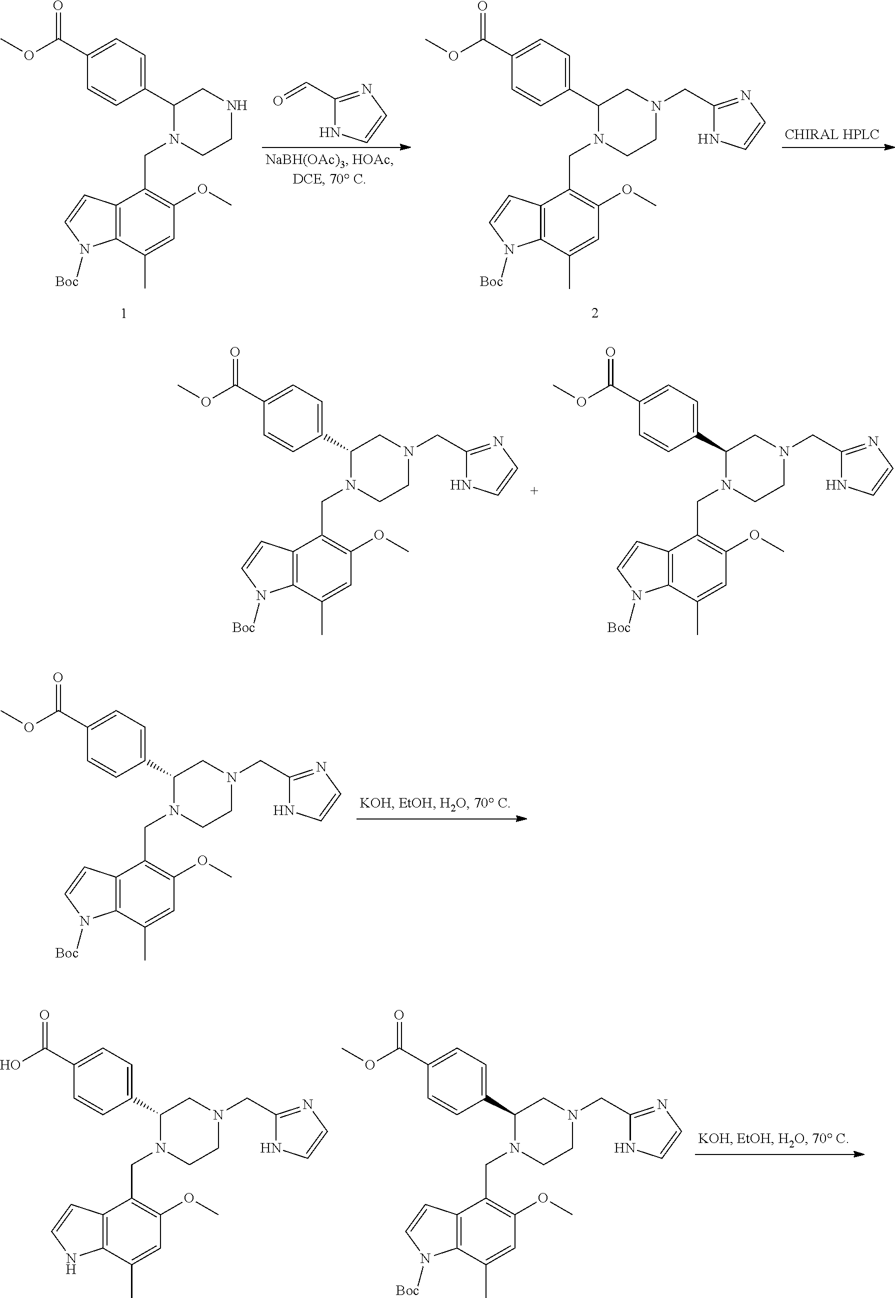

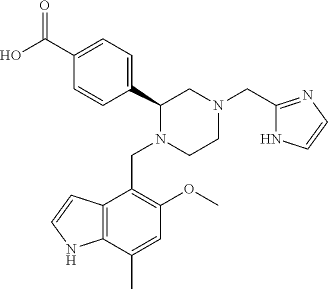 [SMILES: COc1cc(C)c2[nH]ccc2c1CN1CCN(Cc2ncc[nH]2)C[C@@H]1c1ccc(C(=O)O)cc1]

Step 1: tert-Butyl 4-((4-((1H-imidazol-2-yl)methyl)-2-(4-(methoxycarbonyl)phenyl)piperazin-1-yl)methyl)-5-methoxy-7-methyl-1H-indole-1-carboxylate. A mixture of tert-butyl 5-methoxy-4-((2-(4-(methoxycarbonyl)phenyl)piperazin-1-yl)methyl)-7-methyl-1H-indole-1-carboxylate (300 mg, 0.60 mmol), 1H-imidazole-2-carbaldehyde (87 mg, 0.91 mmol), HOAc (37 mg, 0.61 mmol) and sodium triacetoxyborohydride (257 mg, 1.21 mmol) in 1,2-dichloroethane (5 mL) was stirred for 5 h at 70° C. The resulting mixture was concentrated under reduced pressure. The residue was purified by silica gel column chromatography, eluted with petroleum ether/ethyl acetate (7:1) to afford the title compound (175 mg, 50.0% yield) as a yellow solid. LCMS (ESI): [M+H]⁺=574.3.

Step 2: Chiral separation. tert-Butyl 4-((4-((1H-imidazol-2-yl)methyl)-2-(4-(methoxycarbonyl)phenyl)piperazin-1-yl)methyl)-5-methoxy-7-methyl-1H-indole-1-carboxylate (175 mg) was applied for Chiral separation with the following condition: Column: CHIRAL ART Cellulose-SB, 2*25 cm, 5 m; Mobile Phase A: Hex(0.5% 2M NH3-methanol)-HPLC, Mobile Phase B: IPA-HPLC; Flow rate: 20 mL/min; Gradient: 25% B to 25% B in 16 min; Wave Length: 220/254 nm; RT1(min): 10.391; RT2(min): 12.595; Sample Solvent: ethanol-HPLC; Injection Volume: 0.3 mL; Number Of Runs: 16. Isomer 1 (tert-Butyl (S)-4-((4-((1H-imidazol-2-yl)methyl)-2-(4-(methoxycarbonyl)phenyl)piperazin-1-yl)methyl)-5-methoxy-7-methyl-1H-indole-1-carboxylate), faster peak: 60 mg, 34.2% yield, LCMS (ESI): [M+H]⁺=574.3, t_{R}=4.23 min (CHIRAL ART Cellulose-SB (0.46*5 cm, 3 μm), Hex(0.1% DEA): IPA=80: 20, 1.0 mL/min, 254 nm). Isomer 2 (tert-butyl (R)-4-((4-((1H-imidazol-2-yl)methyl)-2-(4-(methoxycarbonyl)phenyl)piperazin-1-yl)methyl)-5-methoxy-7-methyl-1H-indole-1-carboxylate): 56 mg, 32.0% yield, LCMS (ESI): [M+H]⁺=574.3, t_{R}=5.70 min (CHIRAL ART Cellulose-SB (0.46*5 cm, 3 μm), Hex(0.1% DEA): IPA=80: 20, 1.0 mL/min, 254 nm).

Step 3: Enantiomers of 4-(4-((1H-Imidazol-2-yl)methyl)-1-((5-methoxy-7-methyl-1H-indol-4-yl)methyl)piperazin-2-yl)benzoic acid. To a stirred mixture of tert-butyl (R)-4-((4-((1H-imidazol-2-yl)methyl)-2-(4-(methoxycarbonyl)phenyl)piperazin-1-yl)methyl)-5-methoxy-7-methyl-1H-indole-1-carboxylate (56 mg, 0.10 mmol) (isomer 2, slower peak) in water (0.4 mL) and ethanol (2 mL) was added potassium hydroxide (117 mg, 2.08 mmol). The resulting mixture was stirred for 2 h at 70° C. The reaction mixture was acidified to pH=6 with sat. citric acid solution. The solvent was removed under vacuum. The residue was purified by reverse phase chromatography (acetonitrile 10%-30%/10 mM ammonium bicarbonate in water) to afford (R)-4-(4-((1H-Imidazol-2-yl)methyl)-1-((5-methoxy-7-methyl-1H-indol-4-yl)methyl)piperazin-2-yl)benzoic acid (31.9 mg, 71.2% yield) as a white solid (isomer 2). LCMS (ESI): [M+H]=460.2, ¹H NMR (300 MHz, DMSO-d₆) δ 11.92 (brs, 1H), 10.81 (s, 1H), 7.96 (d, J=8.0 Hz, 2H), 7.62 (d, J=7.7 Hz, 2H), 7.25 (t, J=2.8 Hz, 1H), 6.86 (s, 2H), 6.65 (s, 1H), 6.51-6.28 (m, 1H), 3.70 (s, 3H), 3.56 (d, J=11.9 Hz, 1H), 3.46 (d, J=4.1 Hz, 2H), 3.41-3.33 (m, 1H), 3.22 (d, J=11.8 Hz, 1H), 2.77-2.55 (m, 3H), 2.41 (s, 3H), 2.22 (t, J=10.9 Hz, 1H), 2.14-1.92 (m, 2H). To a stirred mixture of tert-butyl (S)-4-((4-((1H-imidazol-2-yl)methyl)-2-(4-(methoxycarbonyl)phenyl)piperazin-1-yl)methyl)-5-methoxy-7-methyl-1H-indole-1-carboxylate (60 mg, 0.10 mmol) (isomer 1, faster peak) in water (0.4 mL) and ethanol (2 mL) was added potassium hydroxide (117 mg, 2.08 mmol). The resulting mixture was stirred for 2 h at 70° C. The reaction mixture was acidified to pH=6 with sat. citric acid solution. The solvent was removed under vacuum. The residue was purified by reverse phase chromatography (acetonitrile 10%-30%/10 mM ammonium bicarbonate in water) to afford (S)-4-(4-((1H-Imidazol-2-yl)methyl)-1-((5-methoxy-7-methyl-1H-indol-4-yl)methyl)piperazin-2-yl)benzoic acid (21.3 mg, 47.5% yield) (isomer 1) as a white solid. LCMS (ESI): [M+H]⁺=460.2, ¹H NMR (300 MHz, DMSO-d₆) δ 11.92 (brs, 1H), 10.81 (s, 1H), 7.96 (d, J=8.0 Hz, 2H), 7.62 (d, J=7.7 Hz, 2H), 7.25 (t, J=2.8 Hz, 1H), 6.86 (s, 2H), 6.65 (s, 1H), 6.51-6.28 (m, 1H), 3.70 (s, 3H), 3.56 (d, J=11.9 Hz, 1H), 3.46 (d, J=4.1 Hz, 2H), 3.41-3.33 (m, 1H), 3.22 (d, J=11.8 Hz, 1H), 2.77-2.55 (m, 3H), 2.41 (s, 3H), 2.22 (t, J=10.9 Hz, 1H), 2.14-1.92 (m, 2H).

Example 47: Enantiomers of (S)-4-(1-((5-Methoxy-7-methyl-1H-indol-4-yl)methyl)-4-(oxazol-2-ylmethyl)piperazin-2-yl)benzoic acid & (R)-4-(1-((5-methoxy-7-methyl-1H-indol-4-yl)methyl)-4-(oxazol-2-ylmethyl)piperazin-2-yl)benzoic acid

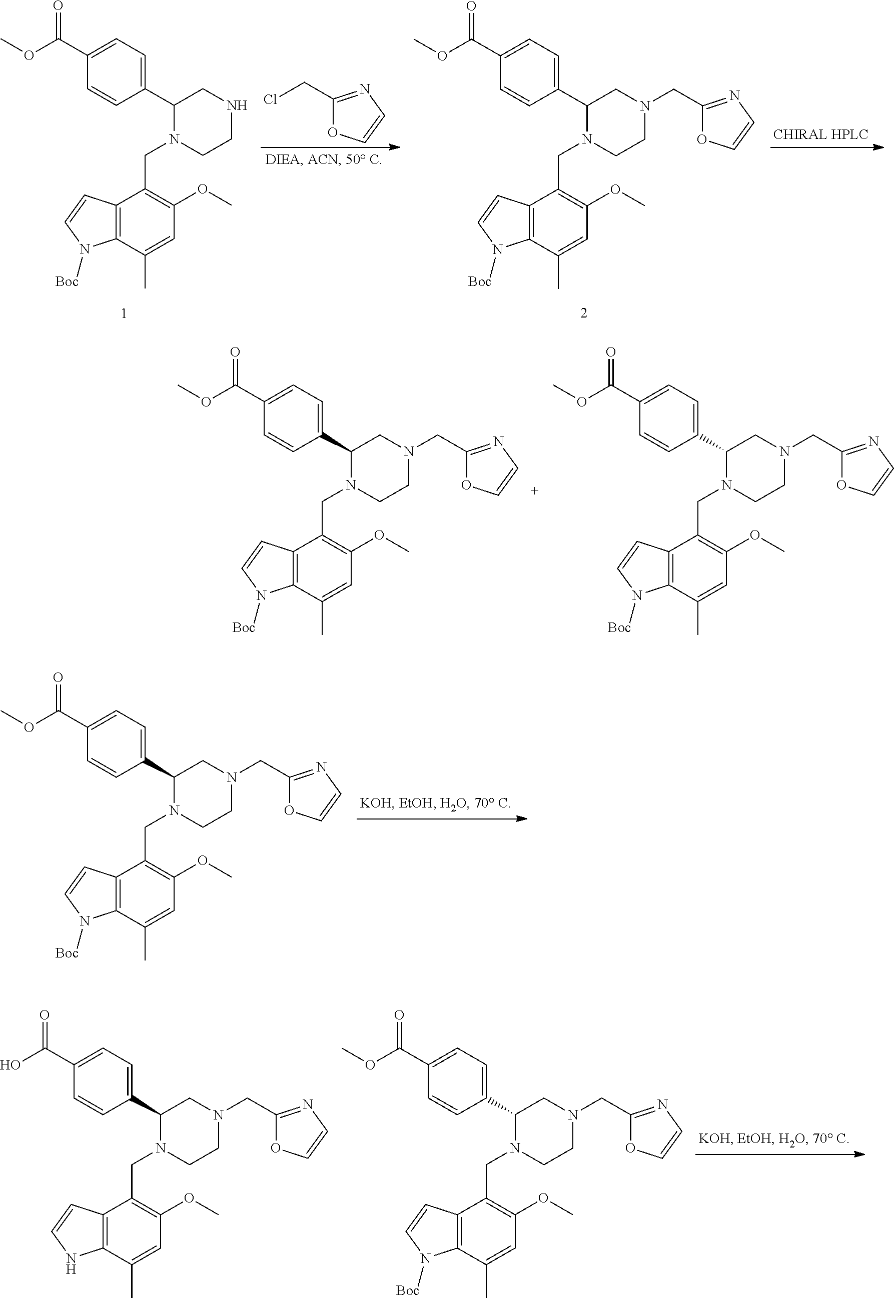

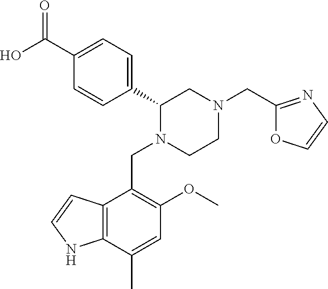 [SMILES: COc1cc(C)c2[nH]ccc2c1CN1CCN(Cc2ncco2)C[C@H]1c1ccc(C(=O)O)cc1]

Step 1: tert-Butyl 5-methoxy-4-((2-(4-(methoxycarbonyl)phenyl)-4-(oxazol-2-ylmethyl)piperazin-1-yl)methyl)-7-methyl-1H-indole-1-carboxylate. A mixture of tert-butyl 5-methoxy-4-((2-(4-(methoxycarbonyl)phenyl)piperazin-1-yl)methyl)-7-methyl-1H-indole-1-carboxylate (500 mg, 1.01 mmol), 2-(chloromethyl)-1,3-oxazole (357 mg, 3.03 mmol) and triethylamine (308 mg, 3.04 mmol) in acetonitrile (5 mL) was stirred for 4 h at 50° C. The resulting mixture was concentrated under reduced pressure. The residue was purified by silica gel column chromatography, eluted with petroleum ether/ethyl acetate (8:1) to afford the title compound (450 mg, 77.4% yield) as a yellow solid. LCMS (ESI): [M+H]⁺=575.3.

Step 2: Enantiomers of tert-Butyl 5-methoxy-4-((2-(4-(methoxycarbonyl)phenyl)-4-(oxazol-2-ylmethyl)piperazin-1-yl)methyl)-7-methyl-1H-indole-1-carboxylate. tert-Butyl 5-methoxy-4-((2-(4-(methoxycarbonyl)phenyl)-4-(oxazol-2-ylmethyl)piperazin-1-yl)methyl)-7-methyl-1H-indole-1-carboxylate (220 mg) was applied for chiral separation with the following condition: Column: CHIRAL ART Cellulose-SB, 2*25 cm, 5 m; Mobile Phase A: Hex(0.5% 2M NH3-methanol)-HPLC, Mobile Phase B: ethanol-HPLC; Flow rate: 20 mL/min; Gradient: 5% B to 5% B in 20 min; Wave Length: 220/254 nm; RT1(min): 14.454; RT2(min): 16.947; Sample Solvent: ethanol-HPLC; Injection Volume: 0.3 mL; Number Of Runs: 24. tert-Butyl (S)-5-methoxy-4-((2-(4-(methoxycarbonyl)phenyl)-4-(oxazol-2-ylmethyl)piperazin-1-yl)methyl)-7-methyl-1H-indole-1-carboxylate (isomer 1, faster peak): 90 mg, 41.0% yield, LCMS (ESI): [M+H]⁺=575.3, t_{R}=4.21 min (CHIRAL Cellulose-SB (0.46*5 cm, 3 μm), Hex(0.1% DEA):ethanol=95:5, 1.0 mL/min, 254 nm). tert-Butyl (R)-5-methoxy-4-((2-(4-(methoxycarbonyl)phenyl)-4-(oxazol-2-ylmethyl)piperazin-1-yl)methyl)-7-methyl-1H-indole-1-carboxylate (isomer 2, slower peak): 90 mg, 41.0% yield, LCMS (ESI): [M+H]⁺=575.3, t_{R}=5.00 min (CHIRAL Cellulose-SB (0.46*5 cm, 3 μm), Hex(0.1% DEA):ethanol=95:5, 1.0 mL/min, 254 nm).

Step 3: Enantiomers of 4-(1-((5-Methoxy-7-methyl-1H-indol-4-yl)methyl)-4-(oxazol-2-ylmethyl)piperazin-2-yl)benzoic acid & (R)-4-(1-((5-methoxy-7-methyl-1H-indol-4-yl)methyl)-4-(oxazol-2-ylmethyl)piperazin-2-yl)benzoic acid. To a stirred mixture of of tert-butyl (S)-5-methoxy-4-((2-(4-(methoxycarbonyl)phenyl)-4-(oxazol-2-ylmethyl)piperazin-1-yl)methyl)-7-methyl-1H-indole-1-carboxylate (90 mg, 0.15 mmol) (isomer 1, faster peak) in water (0.2 mL) and ethanol (2 mL) was added potassium hydroxide (175 mg, 3.11 mmol). The resulting mixture was stirred for 2 h at 70° C. The reaction mixture was acidified to pH=6 with sat. citric acid solution. The solvent was removed under vacuum. The residue was purified by reverse phase chromatography (acetonitrile 10%-30%/10 mM ammonium bicarbonate in water) to afford (S)-4-(1-((5-Methoxy-7-methyl-1H-indol-4-yl)methyl)-4-(oxazol-2-ylmethyl)piperazin-2-yl)benzoic acid (42.5 mg, 58.9% yield) (isomer 1). LCMS (ESI): [M+H]⁺=465.2, ¹H NMR (300 MHz, DMSO-d₆) δ 10.82 (t, J=2.3 Hz, 1H), 8.02 (d, J=0.9 Hz, 1H), 7.96 (d, J=8.0 Hz, 2H), 7.64 (d, J=7.8 Hz, 2H), 7.24 (t, J=2.8 Hz, 1H), 7.11 (d, J=0.9 Hz, 1H), 6.64 (s, 1H), 6.48-6.35 (m, 1H), 3.69 (s, 3H), 3.58 (d, J=26.8 Hz, 3H), 3.44-3.32 (m, 1H), 3.21 (d, J=11.8 Hz, 1H), 2.79-2.57 (m, 3H), 2.40 (s, 3H), 2.28-2.03 (m, 3H). To a stirred mixture of tert-butyl (R)-5-methoxy-4-((2-(4-(methoxycarbonyl)phenyl)-4-(oxazol-2-ylmethyl)piperazin-1-yl)methyl)-7-methyl-1H-indole-1-carboxylate (90 mg, 0.15 mmol) (isomer 2, slower peak) in water (0.2 mL) and ethanol (2 mL) was added potassium hydroxide (175 mg, 3.11 mmol). The resulting mixture was stirred for 2 h at 70° C. The reaction mixture was acidified to pH=6 with sat. citric acid solution. The solvent was removed under vacuum. The residue was purified by reverse phase chromatography (acetonitrile 10%-30%/10 mM ammonium bicarbonate in water) to afford (R)-4-(1-((5-Methoxy-7-methyl-1H-indol-4-yl)methyl)-4-(oxazol-2-ylmethyl)piperazin-2-yl)benzoic acid (34.2 mg, 47.4% yield) (isomer 2). LCMS (ESI): [M+H]⁺=465.2, ¹H NMR (300 MHz, DMSO-d₆) δ 10.82 (t, J=2.3 Hz, 1H), 8.02 (d, J=0.9 Hz, 1H), 7.96 (d, J=8.0 Hz, 2H), 7.64 (d, J=7.8 Hz, 2H), 7.24 (t, J=2.8 Hz, 1H), 7.11 (d, J=0.9 Hz, 1H), 6.64 (s, 1H), 6.48-6.35 (m, 1H), 3.69 (s, 3H), 3.58 (d, J=26.8 Hz, 3H), 3.44-3.32 (m, 1H), 3.21 (d, J=11.8 Hz, 1H), 2.79-2.57 (m, 3H), 2.40 (s, 3H), 2.28-2.03 (m, 3H).

### Example 48: (R)-4-(1-((7-Chloro-5-methoxy-1H-indol-4-yl)methyl)-4-(2,2-difluoroethyl)piperazin-2-yl)benzoic acid

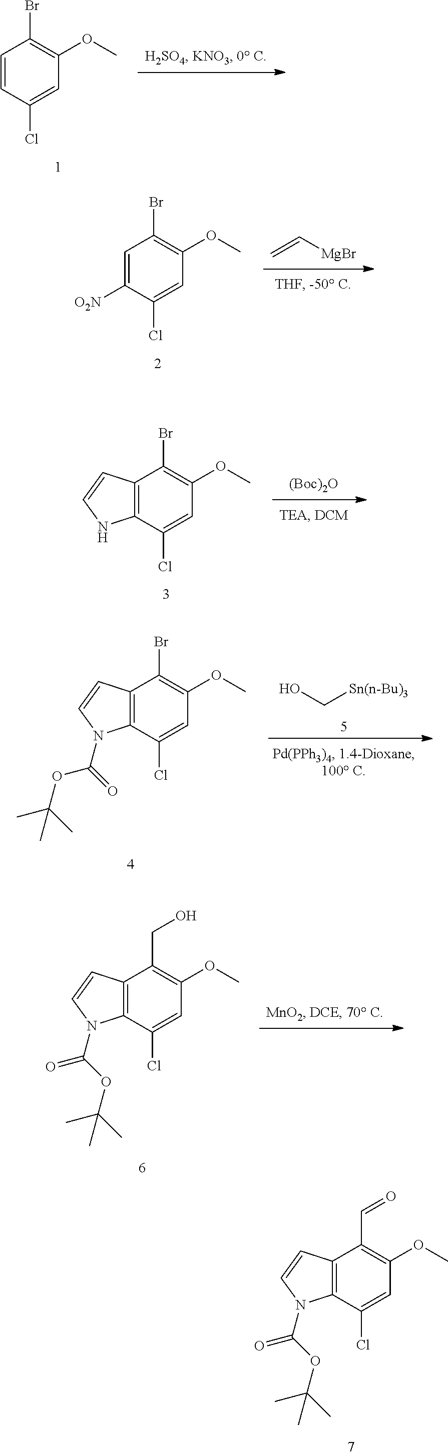

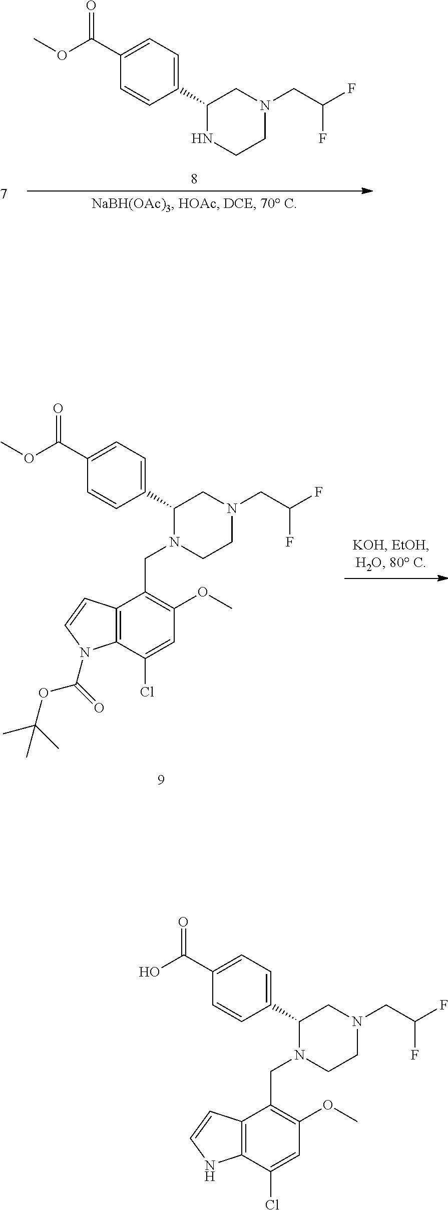

Step 1: 1-Bromo-4-chloro-2-methoxy-5-nitrobenzene. To a mixture of 1-bromo-4-chloro-2-methoxybenzene (25.3 g, 114.2 mmol) in con. H₂SO₄ (120 mL, 98%) was added KNO₃ (8.1 g, 80.0 mmol) in portions at 0° C. and stirred for 1 h at 0° C. The resulting mixture was quenched with ice water. The precipitated solids were collected by filtration and washed with water. The solids were collected and purified by silica gel column chromatography, eluted with ethyl acetate/petroleum ether (1:5) to afford the title compound (9.2 g, 30.2% yield) as a white solid. ¹H NMR (300 MHz, Chloroform-d) δ 8.28 (s, 1H), 6.99 (s, 1H), 4.00 (s, 3H).

Step 2: 4-Bromo-7-chloro-5-methoxy-1H-indole. To a mixture of 1-bromo-4-chloro-2-methoxy-5-nitrobenzene (9.2 g, 34.40 mmol) in tetrahydrofuran (20 mL) was added bromo(ethenyl)magnesium (166 mL, 166 mmol) (1 M in tetrahydrofuran) at −50° C. under nitrogen atmosphere and stirred at the same temperature for 2 h. The reaction was quenched with sat. NH₄Cl at 0° C. The aqueous layer was extracted with ethyl acetate for 3 times. The combined organic layers were combined. The organic layer was dried over anhydrous sodium sulfate. After filtration, the filtrate was concentrated under reduced pressure. The residue was purified by silica gel column chromatography, eluted with ethyl acetate/petroleum ether (1:5) to afford the title compound (2.5 g, 27.3% yield) as a yellow solid. LCMS (ESI): [M+H]⁺=260.1.

Step 3: tert-Butyl 4-bromo-7-chloro-5-methoxyindole-1-carboxylate. To a solution of 4-bromo-7-chloro-5-methoxy-1H-indole (2.5 g, 9.60 mmol) in dichloromethane (50 mL) was added triethylamine (6.6 mL, 47.43 mmol) and di-tert-butyl dicarbonate (5.3 g, 24.28 mmol) at rt. The reaction mixture was stirred at rt for 1 h. The mixture was diluted with dichloromethane and washed with brine. The combined organic layers were dried over anhydrous sodium sulfate. After filtration, the filtrate was concentrated under reduced pressure. The residue was purified by silica gel column chromatography, eluted with ethyl acetate/petroleum ether (1:10) to afford the title compound (3.1 g, 89.6% yield) as a yellow solid. LCMS (ESI): [M+H]⁺=360.1.

Step 4: tert-Butyl 7-chloro-4-(hydroxymethyl)-5-methoxyindole-1-carboxylate. A mixture of tert-butyl 4-bromo-7-chloro-5-methoxyindole-1-carboxylate (662 mg, 1.84 mmol), (tributylstannyl)methanol (897 mg, 2.8 mmol) and tetrakis(triphenylphosphine)palladium (440 mg, 0.38 mmol) in 1,4-dioxane (7 mL) was stirred at 100° C. under nitrogen atmosphere for overnight. The reaction was quenched with sat. KF (aq.) and extracted with EA, washed with brine. The combined organic layers were dried over anhydrous Na₂SO₄. After filtration, the filtrate was concentrated under reduced pressure. The residue was purified by silica gel column chromatography, eluted with ethyl acetate/petroleum ether (1:4) to afford the title compound (500 mg, 87.4% yield) as a white solid. ¹H NMR (300 MHz, Chloroform-d) δ 7.56 (d, J=3.8 Hz, 1H), 6.97 (s, 1H), 6.65 (d, J=3.8 Hz, 1H), 4.88 (s, 2H), 3.90 (s, 3H), 2.26 (s, 1H), 1.64 (s, 9H).

Step 5: tert-Butyl 7-chloro-4-formyl-5-methoxyindole-1-carboxylate. A mixture of tert-butyl 7-chloro-4-(hydroxymethyl)-5-methoxyindole-1-carboxylate (500 mg, 1.60 mmol) in 1,2-dichloroethane (20 mL) was added manganese dioxide (1.4 g, 16.10 mmol) and stirred for 1 h at 70° C. The resulting mixture was filtered, the filter cake was washed with dichloromethane. The filtrate was concentrated under reduced pressure to afford the title compound (400 mg, 80.6% yield) as a yellow solid. LCMS (ESI): [M+H]⁺=310.0.

Step 6: tert-Butyl 7-chloro-4-(((2R)-4-(2,2-difluoroethyl)-2-(4-(methoxycarbonyl)phenyl)piperazin-1-yl)methyl)-5-methoxyindole-1-carboxylate. To a mixture of tert-butyl 7-chloro-4-formyl-5-methoxyindole-1-carboxylate (50 mg, 0.16 mmol), methyl 4-((2R)-4-(2,2-difluoroethyl)piperazin-2-yl)benzoate (50 mg, 0.18 mmol) and HOAc (50 mg, 0.83 mmol) in 1,2-dichloroethane (5 mL) was stirred for 1 day at 70° C. Then sodium triacetoxyborohydride (180 mg, 0.85 mmol) was added in portions and stirred at 70° C. for 5 h. The solvent was removed under reduced pressure. The residue was purified by silica gel column chromatography, eluted with ethyl acetate/petroleum ether (1:4) to afford the title compound (30 mg, 32.1% yield) as a colorless oil. LCMS (ESI): [M+H]⁺=578.1.

Step 7: (R)-4-(1-((7-Chloro-5-methoxy-1H-indol-4-yl)methyl)-4-(2,2-difluoroethyl)piperazin-2-yl)benzoic acid. To a mixture of tert-butyl 7-chloro-4-(((2R)-4-(2,2-difluoroethyl)-2-(4-(methoxycarbonyl)phenyl)piperazin-1-yl)methyl)-5-methoxyindole-1-carboxylate (30 mg, 0.05 mmol) in water (0.75 mL) and ethanol (2 mL) was added potassium hydroxide (30 mg, 0.54 mmol) and stirred for 1 h at 80° C. The mixture was acidified to pH=6 with sat. citric acid. The resulting residue was purified by reverse phase chromatography (2-35% acetonitrile/10 mM NH₄HCO₃ in water) to afford the title compound (8.9 mg, 36.9%) as a white solid. LCMS (ESI): [M+H]⁺=464.1; ¹H NMR (300 MHz, DMSO-d₆) δ 11.22 (s, 1H), 7.98 (d, J=8.0 Hz, 2H), 7.67 (d, J=7.8 Hz, 2H), 7.36 (t, J=2.8 Hz, 1H), 6.95 (s, 1H), 6.63-6.48 (m, 1H), 6.32-5.90 (m, 1H), 3.74 (s, 3H), 3.56 (d, J=12.0 Hz, 1H), 3.46-3.40 (m, 1H), 3.30-3.24 (m, 1H), 2.87-2.55 (m, 5H), 2.33-2.14 (m, 3H).

### Example 49: 4-(1-((5-Cyclopropyl-7-methyl-1H-indol-4-yl)methyl)-4-(2,2,2-trifluoroethyl)piperazin-2-yl)benzoic acid

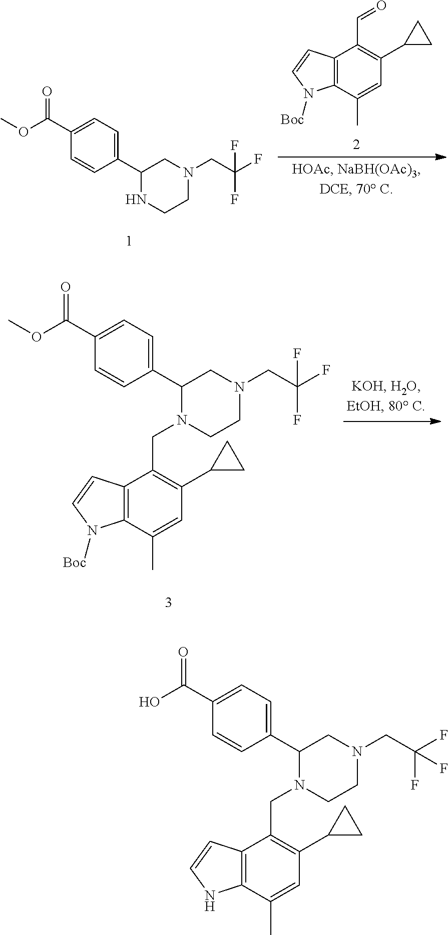 [SMILES: COC(=O)c1ccc(C2CN(CC(F)(F)F)CCN2)cc1 ; Cc1cc(C2CC2)c(C=O)c2ccn(C(=O)OC(C)(C)C)c12 ; COC(=O)c1ccc(C2CN(CC(F)(F)F)CCN2Cc2c(C3CC3)cc(C)c3c2ccn3C(=O)OC(C)(C)C)cc1 ; Cc1cc(C2CC2)c(CN2CCN(CC(F)(F)F)CC2c2ccc(C(=O)O)cc2)c2cc[nH]c12]

Step 1: tert-Butyl 5-cyclopropyl-4-((2-(4-(methoxycarbonyl)phenyl)-4-(2,2,2-trifluoroethyl)piperazin-1-yl)methyl)-7-methylindole-1-carboxylate. A mixture of methyl 4-(4-(2,2,2-trifluoroethyl)piperazin-2-yl)benzoate (50 mg, 0.17 mmol), tert-butyl 5-cyclopropyl-4-formyl-7-methylindole-1-carboxylate (70 mg, 0.23 mmol), HOAc (70 mg, 1.17 mmol) and sodium triacetoxyborohydride (150 mg, 0.71 mmol) in 1,2-Dichloroethane (1 mL) was stirred for overnight at 70° C. The solvent was removed under reduced pressure. The residue was purified by silica gel column chromatography, eluted with ethyl acetate/petroleum ether (1:5) to afford the title compound (40 mg, 41.3% yield) as a colorless oil. LCMS (ESI): [M+H]⁺=586.2.

Step 2: 4-(1-((5-Cyclopropyl-7-methyl-1H-indol-4-yl)methyl)-4-(2,2,2-trifluoroethyl)piperazin-2-yl)benzoic acid. To a mixture of tert-butyl 5-cyclopropyl-4-((2-(4-(methoxycarbonyl)phenyl)-4-(2,2,2-trifluoroethyl)piperazin-1-yl)methyl)-7-methylindole-1-carboxylate (40 mg, 0.07 mmol) in water (0.75 mL) and ethanol (2 mL) was added potassium hydroxide (40 mg, 0.71 mmol) and stirred for 1 h at 80° C. The mixture was acidified to pH=6 with sat. citric acid. The residue was purified by reverse phase flash with the following conditions (5-35% acetonitrile/10 mM NH₄HCO₃ in water) to afford the title compound (7.1 mg, 22.1% yield) as a white solid. LCMS (ESI): [M+H]⁺=472.2; ¹H NMR (300 MHz, DMSO-d₆) δ 12.93 (s, 1H), 10.85 (s, 1H), 7.95 (d, J=7.9 Hz, 2H), 7.65 (d, J=7.9 Hz, 2H), 7.24 (t, J=2.8 Hz, 1H), 6.60-6.50 (m, 1H), 6.46 (s, 1H), 3.74 (d, J=11.9 Hz, 1H), 3.47 (d, J=9.7 Hz, 1H), 3.38 (d, J=11.9 Hz, 1H), 3.27-3.04 (m, 2H), 2.91-2.68 (m, 2H), 2.65-2.53 (m, 3H), 2.36-2.26 (m, 4H), 2.21-2.13 (m, 1H), 0.88-0.77 (m, 1H), 0.75-0.64 (m, 1H), 0.60-0.45 (m, 1H), 0.24-0.10 (m, 1H).

### Example 50: (R)-4-(1-((7-Chloro-5-methoxy-1H-indol-4-yl)methyl)-4-(2,2,2-trifluoroethyl)piperazin-2-yl)benzoic acid

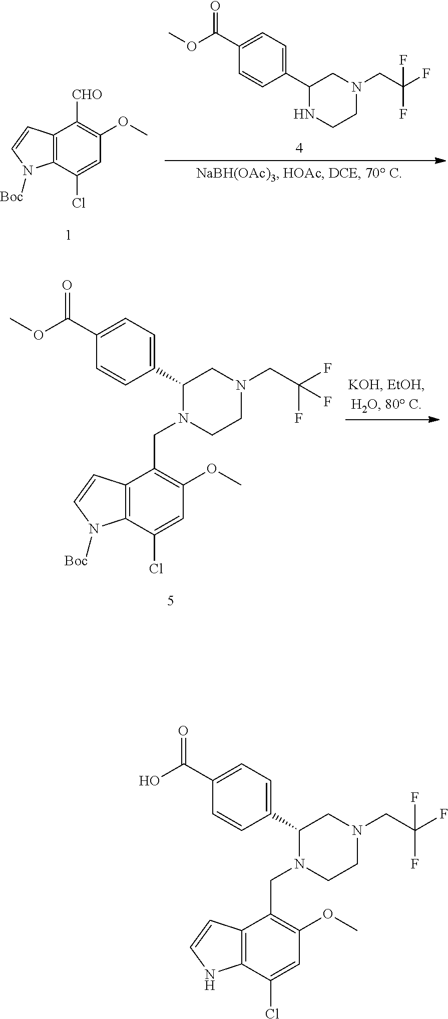 [SMILES: COc1cc(Cl)c2c(ccn2C(=O)OC(C)(C)C)c1C=O ; COC(=O)c1ccc(C2CN(CC(F)(F)F)CCN2)cc1 ; COC(=O)c1ccc([C@@H]2CN(CC(F)(F)F)CCN2Cc2c(OC)cc(Cl)c3c2ccn3C(=O)OC(C)(C)C)cc1 ; COc1cc(Cl)c2[nH]ccc2c1CN1CCN(CC(F)(F)F)C[C@H]1c1ccc(C(=O)O)cc1]

Step 1: tert-Butyl (R)-7-chloro-5-methoxy-4-((2-(4-(methoxycarbonyl)phenyl)-4-(2,2,2-trifluoroethyl)piperazin-1-yl)methyl)-1H-indole-1-carboxylate. To a stirred mixture of tert-butyl 7-chloro-4-formyl-5-methoxyindole-1-carboxylate (50 mg, 0.16 mmol) and methyl (R)-4-(4-(2,2,2-trifluoroethyl)piperazin-2-yl)benzoate (60 mg, 0.19 mmol) in 1,2-dichloroethane (1 mL) was added HOAc (10 mg, 0.16 mmol) and sodium triacetoxyborohydride (100 mg, 0.47 mmol). The resulting mixture was stirred for 6 h at 70° C. The resulting mixture was concentrated under reduced pressure. The residue was purified by silica gel column chromatography, eluted with petroleum ether/ethyl acetate (5:1) to afford the title compound (40 mg, 41.6% yield) as a yellow solid. LCMS (ESI): [M+H]⁺=596.2.

Step 2: (R)-4-(1-((7-Chloro-5-methoxy-1H-indol-4-yl)methyl)-4-(2,2,2-trifluoroethyl)piperazin-2-yl)benzoic acid. A solution of tert-butyl (R)-7-chloro-5-methoxy-4-((2-(4-(methoxycarbonyl)phenyl)-4-(2,2,2-trifluoroethyl)piperazin-1-yl)methyl)-1H-indole-1-carboxylate (40 mg, 0.07 mmol) and potassium hydroxide (94 mg, 1.68 mmol) in ethanol (2 mL) and water (0.4 mL) was stirred for 2 h at 80° C. The residue was acidified to pH=6 with sat. citric acid solution. The residue was purified by reverse flash chromatography with 10% to 50% acetonitrile/10 mmol NH4HCO3) to afford the title compound (17.5 mg, 43.3% yield) as a white solid. LCMS (ESI): [M+H]⁺=482.0, ¹H NMR (300 MHz, DMSO-d₆) δ 12.92 (brs, 1H), 11.23 (s, 1H), 7.98 (t, J=8.7 Hz, 2H), 7.69 (t, J=8.6 Hz, 2H), 7.51-7.26 (m, 1H), 6.96 (d, J=9.5 Hz, 1H), 6.57 (d, J=8.0 Hz, 1H), 3.75 (d, J=8.3 Hz, 3H), 3.57 (t, J=10.6 Hz, 1H), 3.47 (d, J=9.6 Hz, 1H), 3.18 (t, J=9.3 Hz, 3H), 2.91-2.68 (m, 2H), 2.61-2.57 (m, 1H), 2.39-2.23 (m, 3H).

### Example 51: 4-(1-((7-Chloro-5-methoxy-1H-indol-4-yl)methyl)-4-(2,2,2-trifluoroethyl)piperazin-2-yl)benzoic acid

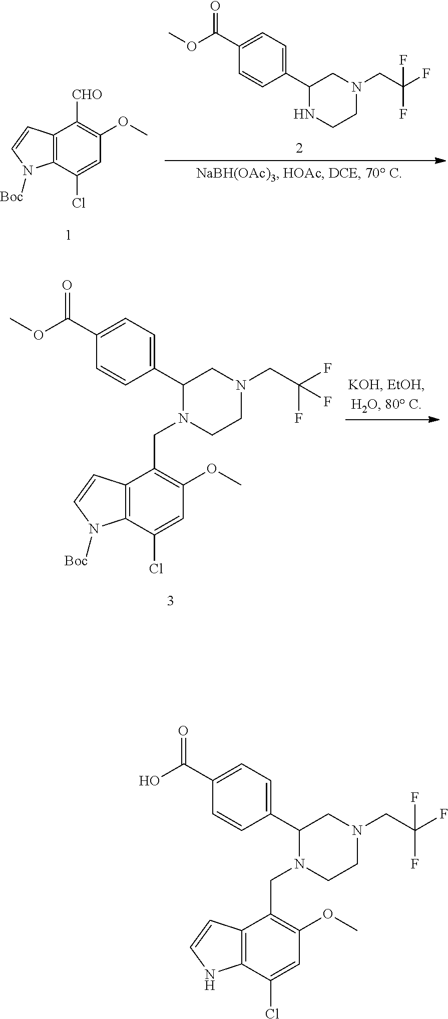 [SMILES: COc1cc(Cl)c2c(ccn2C(=O)OC(C)(C)C)c1C=O ; COC(=O)c1ccc(C2CN(CC(F)(F)F)CCN2)cc1 ; COC(=O)c1ccc(C2CN(CC(F)(F)F)CCN2Cc2c(OC)cc(Cl)c3c2ccn3C(=O)OC(C)(C)C)cc1 ; COc1cc(Cl)c2[nH]ccc2c1CN1CCN(CC(F)(F)F)CC1c1ccc(C(=O)O)cc1]

Step 1: tert-Butyl 7-chloro-5-methoxy-4-((2-(4-(methoxycarbonyl)phenyl)-4-(2,2,2-trifluoroethyl)piperazin-1-yl)methyl)-1H-indole-1-carboxylate. To a stirred mixture of tert-butyl 7-chloro-4-formyl-5-methoxyindole-1-carboxylate (50 mg, 0.16 mmol) and methyl 4-(4-(2,2,2-trifluoroethyl)piperazin-2-yl)benzoate (60 mg, 0.19 mmol) in 1,2-dichloroethane (1 mL) was added HOAc (10 mg, 0.16 mmol) and sodium triacetoxyborohydride (100 mg, 0.47 mmol). The resulting mixture was stirred for 6 h at 70° C. The resulting mixture was concentrated under reduced pressure. The residue was purified by silica gel column chromatography, eluted with petroleum ether/ethyl acetate (5:1) to afford the title compound (42 mg, 43.5% yield) as a yellow solid. LCMS (ESI): [M+H]⁺=596.2.

Step 2: 4-(1-((7-Chloro-5-methoxy-1H-indol-4-yl)methyl)-4-(2,2,2-trifluoroethyl)piperazin-2-yl)benzoic acid. A solution of tert-butyl 7-chloro-5-methoxy-4-((2-(4-(methoxycarbonyl)phenyl)-4-(2,2,2-trifluoroethyl)piperazin-1-yl)methyl)-1H-indole-1-carboxylate (42 mg, 0.07 mmol) and potassium hydroxide (98 mg, 1.68 mmol) in ethanol (2 mL) and water (0.4 mL) was stirred for 2 h at 80° C. The residue was acidified to pH=6 with sat. citric acid solution. The residue was purified by reverse flash chromatography with 10% to 50% acetonitrile/10 mmol NH4HCO3) to afford the title compound (20.5 mg, 60.3% yield) as a white solid. LCMS (ESI): [M+H]⁺=482.1, ¹H NMR (300 MHz, DMSO-d₆) δ 12.92 (brs, 1H), 11.23 (s, 1H), 7.98 (t, J=8.7 Hz, 2H), 7.69 (t, J=8.6 Hz, 2H), 7.51-7.26 (m, 1H), 6.96 (d, J=9.5 Hz, 1H), 6.57 (d, J=8.0 Hz, 1H), 3.75 (d, J=8.3 Hz, 3H), 3.57 (t, J=10.6 Hz, 1H), 3.47 (d, J=9.6 Hz, 1H), 3.18 (t, J=9.3 Hz, 3H), 2.91-2.68 (m, 2H), 2.61-2.57 (m, 1H), 2.39-2.23 (m, 3H).

### Example 52: (S)-4-(4-(2,2-Difluoroethyl)-1-((5-methoxy-7-methyl-1H-indol-4-yl)methyl)piperazin-2-yl)benzoic acid

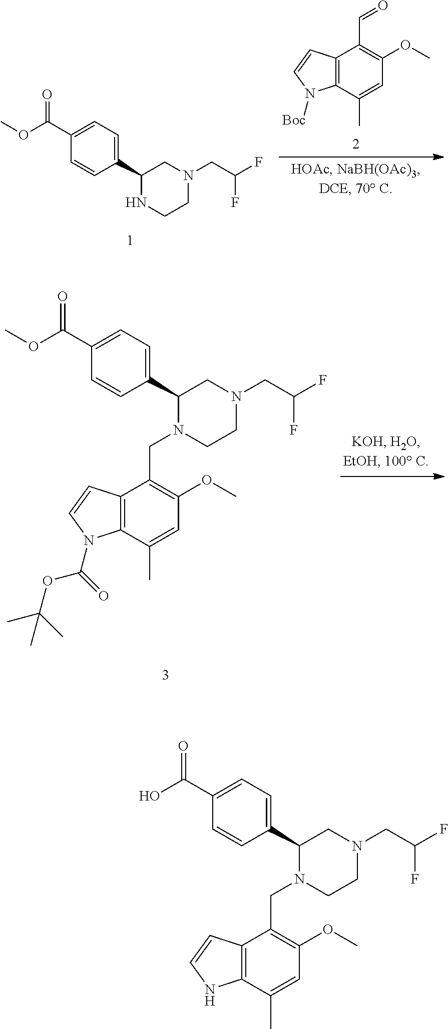 [SMILES: COC(=O)c1ccc([C@H]2CN(CC(F)F)CCN2)cc1 ; COc1cc(C)c2c(ccn2C(=O)OC(C)(C)C)c1C=O ; COC(=O)c1ccc([C@H]2CN(CC(F)F)CCN2Cc2c(OC)cc(C)c3c2ccn3C(=O)OC(C)(C)C)cc1 ; COc1cc(C)c2[nH]ccc2c1CN1CCN(CC(F)F)C[C@@H]1c1ccc(C(=O)O)cc1]

Step 1: tert-Butyl (S)-4-((4-(2,2-difluoroethyl)-2-(4-(methoxycarbonyl)phenyl)piperazin-1-yl)methyl)-5-methoxy-7-methyl-1H-indole-1-carboxylate. A solution of tert-butyl 4-formyl-5-methoxy-7-methylindole-1-carboxylate (280 mg, 0.99 mmol) in 1,2-dichloroethane (6 mL) was added methyl 4-((2S)-4-(2,2-difluoroethyl)piperazin-2-yl)benzoate (140 mg, 0.49 mmol), sodium triacetoxyborohydride (210 mg, 0.99 mmol) and HOAc (28 mg, 0.47 mmol) at rt. The resulting mixture was stirred at 70° C. for overnight. The resulting mixture was concentrated under reduced pressure. The residue was purified by flash chromatography on silica gel eluting with petroleum ether/ethyl acetate (4:1) to afford the title compound (160 mg, 58.3%) as a yellow oil. LCMS (ESI): [M+H]⁺=558.3.

Step 2: (S)-4-(4-(2,2-Difluoroethyl)-1-((5-methoxy-7-methyl-1H-indol-4-yl)methyl)piperazin-2-yl)benzoic acid. A mixture of tert-butyl (S)-4-((4-(2,2-difluoroethyl)-2-(4-(methoxycarbonyl)phenyl)piperazin-1-yl)methyl)-5-methoxy-7-methyl-1H-indole-1-carboxylate (160 mg, 0.29 mmol) and potassium hydroxide (160 mg, 2.85 mmol) in water (0.25 mL) and ethanol (5 mL) was stirred for 1 h at 100° C. The reaction mixture was acidified to pH=6 with sat. citric acid. The mixture was purified by reverse flash chromatography with 10% to 30% MeCN/10 mmol/L NH₄HCO₃ to afford the title compound (64.3 mg, 50.5% yield) as a white solid. LCMS (ESI): [M+H]⁺=444.1, ¹H NMR (300 MHz, DMSO-d₆) δ 10.84 (s, 1H), 8.02-7.94 (m, 2H), 7.69 (d, J=7.8 Hz, 2H), 7.26 (t, J=2.8 Hz, 1H), 6.66 (s, 1H), 6.46-6.42 (m, 1H), 6.32-5.90 (m, 1H), 3.71 (s, 3H), 3.60-3.53 (m, 1H), 3.45-3.39 (m, 1H), 3.27-3.19 (m, 1H), 2.86-2.72 (m, 3H), 2.71-2.67 (m, 1H), 2.66-2.59 (m, 1H), 2.42 (s, 3H), 2.30-2.16 (m, 3H).

### Example 53: tert-Butyl (R)-5-methoxy-4-((2-(4-(methoxycarbonyl)phenyl)piperazin-1-yl)methyl)-7-methyl-1H-indole-1-carboxylate

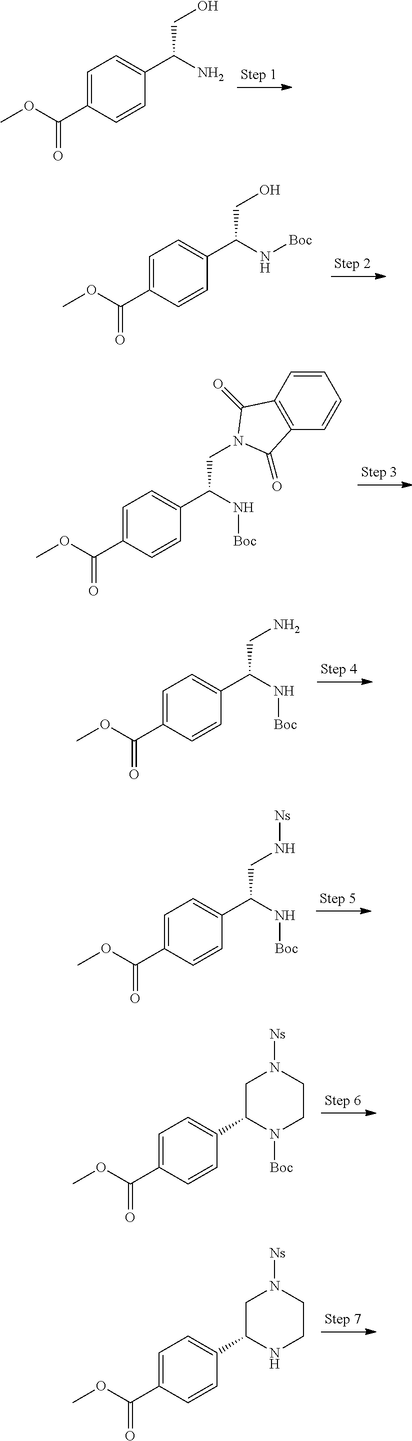

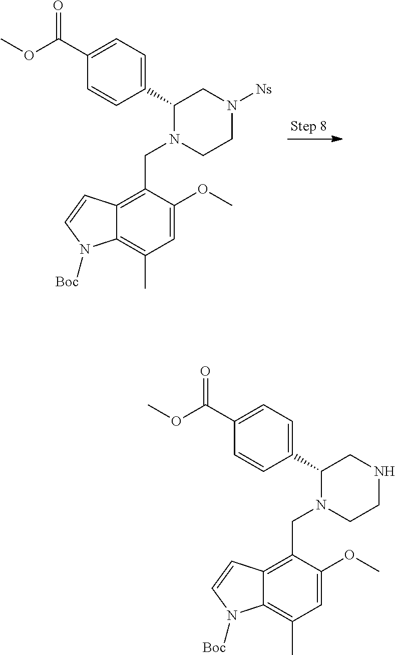

Step 1: Methyl (R)-4-(1-((tert-butoxycarbonyl)amino)-2-hydroxyethyl)benzoate was prepared with the following procedure. Methyl (R)-4-(1-amino-2-hydroxyethyl)benzoate hydrochloride (200 g, 863 mmol. 1.0 eq.) was suspended in DCM (3 L) and cooled to 0° C. Boc₂O (207 g, 950 mmol, 1.1 eq.) and Et₃N (265 mL, 1.90 mol, 2.2 eq.) were added and the mixture was stirred for 15 h, allowing to reach room temperature. The reaction was worked up; the solution was washed with sat. aq. NaHCO₃, brine, dried over Na₂SO₄ and concentrated to afford methyl (R)-4-(1-((tert-butoxycarbonyl)amino)-2-hydroxyethyl)benzoate (260 g, 97%) as a white solid. ¹H-NMR (300 MHz, CDCl₃) δ 8.02 (d, J=8.3 Hz, 2H), 7.38 (d, J=8.3 Hz, 2H), 5.36 (br. s, 1H), 4.81 (br. s, 1H), 3.91 (s, 3H), 3.86 (d, J=5.0 Hz, 2H), 2.16 (br. s, 1H), 1.52 (s, 1H), 1.44 (d, J=7.7 Hz, 9H), 1.27 (s, 1H).

Step 2: Methyl (R)-4-(1-((tert-butoxycarbonyl)amino)-2-(1,3-dioxoisoindolin-2-yl)ethyl)benzoate was prepared with the following procedure. To a solution of methyl (R)-4-(1-((tert-butoxycarbonyl)amino)-2-hydroxyethyl)benzoate (260.0 g, 836.3 mmol, 1.0 eq.), phthalimide (246.1 g, 1.673 mol, 2.0 eq.), and PPh₃ (329.0 g, 1.254 mol, 1.5 eq.) in THF (3 L) under N2 at 0° C. was dropwise added DIAD (284.6 mL, 1.464 mol, 1.75 eq.). After 15 min was the yellow solution warmed to 20° C. A solid precipitate formed and the thick suspension was diluted with THE (1 L). The mixture was stirred for 3 hours in total at 20° C. The mixture was diluted with EtOAc (4 L) and 2 M aq. NaOH (1.5 L) was added. The mixture is filtered (overnight) and the solid washed with water (8 L), TBME (8 L) and triturated in EtOAc (6 L), filtered and dried under reduced pressure affording 260 g of methyl (R)-4-(1-((tert-butoxycarbonyl)amino)-2-(1,3-dioxoisoindolin-2-yl)ethyl)benzoate (the material still contains some water). All water and organic solvents were combined and the organic layer separated. The aqueous layer was extracted with EtOAc (3 L). The combined organic layers were washed with brine, dried over Na₂SO₄, and concentrated under reduced pressure. The solid residue was triturated in TBME (6 L) and dried under reduced pressure affording 100 g of methyl (R)-4-(1-((tert-butoxycarbonyl)amino)-2-(1,3-dioxoisoindolin-2-yl)ethyl)benzoate. The crops were separately converted in the next step. ¹H-NMR (300 MHz, CDCl₃) δ 8.03 (d, J=8.3 Hz, 2H), 7.86 (dd, J=5.4, 3.1 Hz, 2H), 7.73 (dd, J=5.5, 3.1 Hz, 2H), 7.45 (d, J=8.3 Hz, 2H), 5.50 (br. s, 1H), 5.13 (br. s, 1H), 3.99-3.87 (m, 5H), 1.28 (s, 9H).

Step 3: Methyl (R)-4-(2-amino-1-((tert-butoxycarbonyl)amino)ethyl)benzoate was prepared with the following procedure. To a mixture of methyl (R)-4-(1-((tert-butoxycarbonyl)amino)-2-(1,3-dioxoisoindolin-2-yl)ethyl)benzoate (100 g, 236 mmol, 1.0 eq.) in IPA (2.0 L) was added hydrazine hydrate (231 mL, 64% wt, 4.71 mol, 20 eq.) and the mixture was stirred for 30 min at 70° C. Initially, all dissolved (40° C.) but then (60° C.) a white precipitate formed. HPLC after 30 min at 70° C. showed full conversion. The reaction was worked up; the mixture was cooled to room temperature using an ice/salt bath. The precipitate was filtered off and the mother liquor was concentrated. The residue was taken up in chloroform, filtered and concentrated affording methyl (R)-4-(2-amino-1-((tert-butoxycarbonyl)amino)ethyl)benzoate (60.3 g, 87%) as white solid. ¹H-NMR (300 MHz, CDCl₃) δ 8.00 (d, J=8.3 Hz, 2H), 7.35 (d, J=8.3 Hz, 2H), 5.55 (app. t, J=7.7 Hz, 1H), 4.69 (br. s, 1H), 3.90 (s, 3H), 3.00 (d, J=5.4 Hz, 2H), 1.41 (s, 9H), 1.25 (d, J=6.3 Hz, 1H).

Step 4: Methyl (R)-4-(1-((tert-butoxycarbonyl)amino)-2-((2-nitrophenyl)sulfonamido)ethyl)benzoate was prepared with the following procedure. To a suspension of methyl (R)-4-(2-amino-1-((tert-butoxycarbonyl)amino)ethyl)benzoate (60.3 g, 184 mmol, 1.0 eq.) and DIPEA (41.8 mL, 240 mmol, 1.3 eq.) in DCM (2.0 L) was cooled to 0° C. and stirred at this temperature for 10 min before 2-nitrobenzene sulfonyl chloride (42.9 g, 194 mmol, 1.05 eq.) was added. After 15 min at the same temperature was the reaction warmed to room temperature. The mixture was stirred for 2 hours at room temperature and then quenched with sat. aq. NaHCO₃ (1.0 L). The organic layer was separated and the aqueous layer extracted with DCM (0.5 L). The combined organic layers were washed with water, dried over Na₂SO₄, and concentrated under reduced pressure. The crude material was purified by means of column chromatography (2 kg silica; heptanes/EtOAc 3:1 to 1:1) affording methyl (R)-4-(1-((tert-butoxycarbonyl)amino)-2-((2-nitrophenyl)sulfonamido)ethyl)benzoate (67.0 g, 76%) as a white solid. ¹H-NMR (300 MHz, CDCl₃) δ 8.14-8.03 (m, 1H), 8.03-7.93 (m, 2H), 7.90-7.80 (m, 1H), 7.80-7.66 (m, 3H), 7.30 (d, J=8.3 Hz, 3H), 5.72 (app. t, J=6.3 Hz, 1H), 5.27 (br. s, 1H), 4.81 (br. s, 1H), 3.91 (s, 3H), 3.64-3.45 (m, 1H), 3.45-3.28 (m, 1H), 1.61 (s, 1H), 1.40 (s, 9H).

Step 5: tert-Butyl (R)-2-(4-(methoxycarbonyl)phenyl)-4-((2-nitrophenyl)sulfonyl)piperazine-1-carboxylate was prepared with the following procedure. Methyl (R)-4-(1-((tert-butoxycarbonyl)amino)-2-((2-nitrophenyl)sulfonamido)ethyl)benzoate (67.0 g, 140 mmol, 1.0 eq.) was dissolved in DCM (2.0 L) and cooled to 0° C. (2-Bromoethyl)diphenylsulfonium trifluoromethanesulfonate (77.4 g, 175 mmol, 1.25 eq.) was added followed by the dropwise addition of DIPEA (73.0 mL, 419 mmol 3.0 eq.) at 0° C. The mixture was stirred at room temperature overnight and then concentrated under reduced pressure. The residue was triturated with 20% EtOAc/heptanes (1 L). The solid was collected by filtration, washed with 20% EtOAc/heptanes (250 mL), and dried under reduced pressure. The intermediate was dissolved in THE (1.5 L) cooled to 0° C. and NaH (16.8 g, 60% Wt, 419 mmol, 3.0 eq.) was added portionwise. The mixture was stirred at 0° C. for 30 min and then allowed to warm to room temperature and stirred for 2 hours. The mixture was cooled to 10° C. and carefully quenched with sat. aq. NaHCO₃ (1.5 L) and then extracted with EtOAc (2×1.5 L). The combined organic layers were washed with brine, dried over Na₂SO₄ and concentrated under reduced pressure. The crude material was purified by means of column chromatography (2 kg silica; heptanes/EtOAc 3:1 to 1:1) affording tert-butyl (R)-2-(4-(methoxycarbonyl)phenyl)-4-((2-nitrophenyl)sulfonyl)piperazine-1-carboxylate (59.0 g, 84%) as white foam. ¹H-NMR (300 MHz, CDCl₃) δ 7.97 (d, J=8.5 Hz, 2H), 7.90 (dd, J=7.5, 1.9 Hz, 1H), 7.78-7.52 (m, 3H), 7.45-7.33 (m, 2H), 5.42 (br. s, 1H), 4.40-4.25 (m, 1H), 4.19-4.02 (m, 1H), 3.91 (s, 3H), 3.84-3.69 (m, 1H), 3.25 (dd, J=13.0, 4.2 Hz, 1H), 3.10 (ddd, J=13.6, 11.9, 3.4 Hz, 1H), 2.90 (td, J=11.8, 3.4 Hz, 1H), 1.57 (s, 1H), 1.44 (s, 9H).

Step 6: (R)-2-(4-(Methoxycarbonyl)phenyl)-4-((2-nitrophenyl)sulfonyl)piperazin-1-ium 2,2,2-trifluoroacetate was prepared with the following procedure. tert-Butyl (R)-2-(4-(methoxycarbonyl)phenyl)-4-((2-nitrophenyl)sulfonyl)piperazine-1-carboxylate (59.0 g, 117 mmol, 1.0 eq.) was dissolved in DCM (1.0 L) and cooled to 0° C. TFA (169 mL, 2.19 mol, 18.8 eq.) was added slowly at 0° C. and then the mixture was stirred at room temperature for 18 hours. The mixture was concentrated under reduced pressure and stripped with toluene (3×0.5 L) affording (R)-2-(4-(methoxycarbonyl)phenyl)-4-((2-nitrophenyl)sulfonyl)piperazin-1-ium 2,2,2-trifluoroacetate (73.0 g, 99%) as an off-white foam. ¹H-NMR (300 MHz, CDCl₃) δ 8.14-7.91 (m, 3H), 7.88-7.64 (m, 3H), 7.56-7.37 (m, 2H), 7.31-7.08 (m, 2H), 4.33 (dd, J=11.3, 3.4 Hz, 1H), 4.17-4.02 (m, 1H), 4.02-3.86 (m, 3H), 3.52 (dd, J=14.1, 11.3 Hz, 1H), 3.37 (ddd, J=14.2, 10.3, 4.6 Hz, 1H), 3.27-3.06 (m, 2H), 2.36 (s, 1H). ¹⁹F-NMR (282 MHz, CDCl₃) δ −75.93.

Step 7: tert-Butyl (R)-5-methoxy-4-((2-(4-(methoxycarbonyl)phenyl)-4-((2-nitrophenyl)sulfonyl)piperazin-1-yl)methyl)-7-methyl-1H-indole-1-carboxylate was prepared in three batches. A typical procedure for one batch is as follows: methyl (R)-4-(4-((2-nitrophenyl)sulfonyl)piperazin-2-yl)benzoate 2,2,2-trifluoroacetate (38.7 g, 61.1 mmol, 1.0 eq.) was mixed with tert-butyl 4-formyl-5-methoxy-7-methyl-1H-indole-1-carboxylate (26.5 g, 91.6 mmol, 1.5 eq.) in THE (1.0 L) and allowed to stir at room temperature under N₂ for 10 minutes. Then Ti(iOPr)₄ (32.8 mL, 108 mmol, 1.8 eq.) was added and the mixture was allowed to stir for 3 hours before adding NaBH(OAc)₃ (51.8 g, 244 mmol, 4.0 eq.). The resulting mixture was allowed to stir at room temperature for 18 hours. The reaction was worked up; the mixture was quenched with sat. aq. NaHCO₃ (1.3 L) and the layers were separated. The aqueous layer was extracted with EtOAc (2×1.0 L) and the combined organic layers were washed with brine and dried over Na₂SO₄, filtered and concentrated under reduced pressure to afford crude tert-butyl (R)-5-methoxy-4-((2-(4-(methoxycarbonyl)phenyl)-4-((2-nitrophenyl)sulfonyl)piperazin-1-yl)methyl)-7-methyl-1H-indole-1-carboxylate (54 g) as an off-white foam. This batch was combined with the two other batches and purified by normal phase FCC (3 kg of silica; Heptanes/EtOAc 4:1 to 2:1) affording of tert-butyl (R)-5-methoxy-4-((2-(4-(methoxycarbonyl)phenyl)-4-((2-nitrophenyl)sulfonyl)piperazin-1-yl)methyl)-7-methyl-1H-indole-1-carboxylate (76.5 g, 98%) as yellowish foam. ¹H-NMR (300 MHz, CDCl₃) δ 8.14-7.98 (m, 2H), 7.94-7.82 (m, 1H), 7.73-7.52 (m, 5H), 7.47 (d, J=3.8 Hz, 1H), 6.67 (s, 1H), 6.50 (d, J=3.8 Hz, 1H), 3.93 (s, 3H), 3.79 (s, 3H), 3.78-3.57 (m, 4H), 3.49 (dd, J=10.5, 3.2 Hz, 1H), 3.30 (d, J=12.1 Hz, 1H), 2.85 (td, J=11.0, 10.1, 7.0 Hz, 3H), 2.58 (s, 3H), 2.42-2.21 (m, 1H), 2.04 (s, OH), 1.61 (s, 10H), 1.59 (d, J=10.1 Hz, 2H), 1.43 (s, OH), 1.37-1.17 (m, 6H), 0.95 (d, J=6.3 Hz, 1H), 0.92-0.78 (m, 5H).

Step 8: tert-Butyl (R)-5-methoxy-4-((2-(4-(methoxycarbonyl)phenyl)piperazin-1-yl)methyl)-7-methyl-1H-indole-1-carboxylate was prepared with the following procedure. To a suspension of tert-butyl (R)-5-methoxy-4-((2-(4-(methoxycarbonyl)phenyl)-4-((2-nitrophenyl)sulfonyl)piperazin-1-yl)methyl)-7-methyl-1H-indole-1-carboxylate (51.2 g, 75.4 mmol, 1.0 eq.) and K₂CO₃ (31.3 g, 226 mmol, 3.0 eq.) in CH₃CN (750 mL) under N₂ was added thiophenol (23.3 mL, 226 mmol, 3.0 eq.). The resulting mixture was stirred at room temperature for 7 hours. The reaction was worked up; the reaction mixture was diluted with EtOAc (1.0 L) and washed with brine, dried over Na₂SO₄, and concentrated under reduced pressure. The crude was purified by filtration over 750 g of Silica. First the side-products were eluted with DCM, then the product is eluted with DCM+10% 7N NH3 in MeOH affording tert-butyl (R)-5-methoxy-4-((2-(4-(methoxycarbonyl)phenyl)piperazin-1-yl)methyl)-7-methyl-1H-indole-1-carboxylate (30.0 g, 81%) as off-white foam. Chiral SFC (IC3 grad MA1) RT: 7.36 min; area % (254 nm): >99% ee; m/z=494.2 [M+H]⁺. ¹H-NMR (300 MHz, CDCl₃) δ 8.05 (d, J=8.6 Hz, 2H), 7.63 (d, J=7.8 Hz, 2H), 7.49 (d, J=3.8 Hz, 1H), 6.67 (s, 1H), 6.62 (d, J=3.8 Hz, 1H), 3.92 (s, 2H), 3.80 (s, 3H), 3.66 (d, J=12.2 Hz, 1H), 3.41-3.22 (m, 2H), 3.06-2.86 (m, 2H), 2.80 (ddt, J=12.6, 10.3, 2.5 Hz, 3H), 2.58 (s, 3H), 2.29-2.01 (m, 3H), 1.61 (s, 9H). An aliquot was converted according to Example 39 and gave biological activity similar to the product of Example 39.

### Example 54: (R)-4-(1-((5-Methoxy-7-methyl-1H-indol-4-yl)methyl)-4-(2,2,2-trifluoroethyl)piperazin-2-yl)benzoic acid

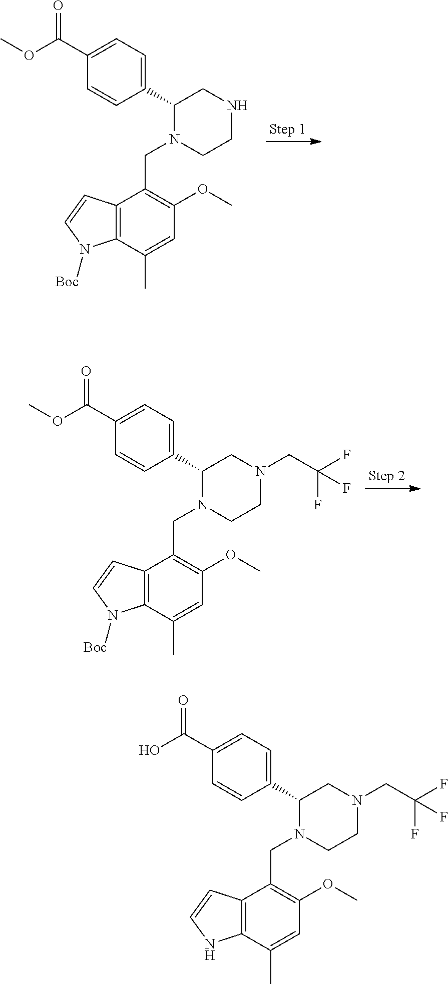

Step 1. tert-Butyl (R)-5-methoxy-4-((2-(4-(methoxycarbonyl)phenyl)-4-(2,2,2-trifluoroethyl)piperazin-1-yl)methyl)-7-methyl-1H-indole-1-carboxylate was prepared according to General Experimental Procedure 3. tert-Butyl (R)-5-methoxy-4-((2-(4-(methoxycarbonyl)phenyl)piperazin-1-yl)methyl)-7-methyl-1H-indole-1-carboxylate (150 mg, 304 μmol) and 2,2,2-trifluoroethyl trifluoromethanesulfonate (65.7 μL, 456 μmol) gave tert-butyl (R)-5-methoxy-4-((2-(4-(methoxycarbonyl)phenyl)-4-(2,2,2-trifluoroethyl)piperazin-1-yl)methyl)-7-methyl-1H-indole-1-carboxylate (145 mg, 83%) as a glassy white solid. LCMS (General 3) RT: 1.37 min; area % (254 nm): 94%; m/z=576.44 [M+H]⁺.

Step 2. (R)-4-(1-((5-Methoxy-7-methyl-1H-indol-4-yl)methyl)-4-(2,2,2-trifluoroethyl)piperazin-2-yl)benzoic acid was prepared according to General Experimental Procedure 2. tert-Butyl (R)-5-methoxy-4-((2-(4-(methoxycarbonyl)phenyl)-4-(2,2,2-trifluoroethyl)piperazin-1-yl)methyl)-7-methyl-1H-indole-1-carboxylate (145 mg, 252 μmol) gave (R)-4-(1-((5-methoxy-7-methyl-1H-indol-4-yl)methyl)-4-(2,2,2-trifluoroethyl)piperazin-2-yl)benzoic acid (75 mg, 64%) as an off-white solid. LCMS (31697 LCMS-5 C3) RT: 2.101 min; area % (215 nm): 96.8%; m/z=462.2 [M+H]⁺. ¹H-NMR (400 MHz, DMSO-d₆) δ 10.83 (t, J=2.4 Hz, 1H), 7.98 (d, J=7.9 Hz, 2H), 7.66 (d, J=7.7 Hz, 2H), 7.25 (t, J=2.8 Hz, 1H), 6.65 (s, 1H), 6.44 (dd, J=3.1, 1.9 Hz, 1H), 3.71 (s, 3H), 3.57 (d, J=11.8 Hz, 1H), 3.49-3.34 (m, 2H), 3.34-2.99 (m, 4H), 2.79 (t, J=12.0 Hz, 2H), 2.73-2.57 (m, 1H), 2.44-2.30 (m, 5H), 2.30-2.15 (m, 1H). ¹⁹F-NMR-HDEC (282 MHz, DMSO-d₆) δ −67.84.

### Example 55: Enantiomer of 4-(4-(2,2-difluoroethyl)-1-((5-methoxy-7-methyl-1H-indol-4-yl)methyl)piperazin-2-yl)-N-(methylsulfonyl)benzamide

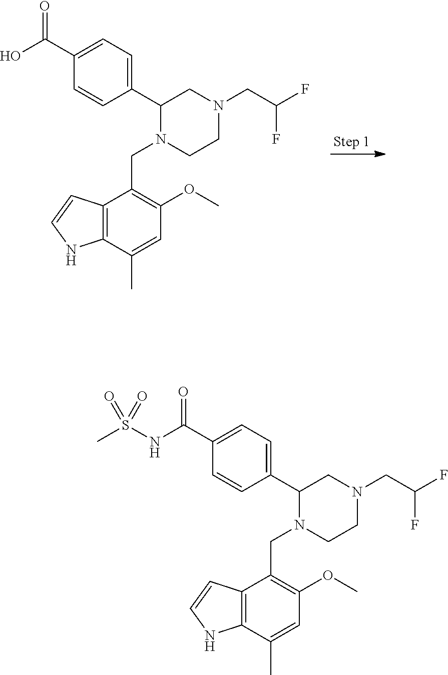 [SMILES: COc1cc(C)c2[nH]ccc2c1CN1CCN(CC(F)F)CC1c1ccc(C(=O)O)cc1 ; COc1cc(C)c2[nH]ccc2c1CN1CCN(CC(F)F)CC1c1ccc(C(=O)NS(C)(=O)=O)cc1]

Step 1. Enantiomer of 4-(4-(2,2-difluoroethyl)-1-((5-methoxy-7-methyl-1H-indol-4-yl)methyl)piperazin-2-yl)-N-(methylsulfonyl)benzamide was prepared according to General Experimental Procedure 4: Enantiomer of 4-(4-(2,2-difluoroethyl)-1-((5-methoxy-7-methyl-1H-indol-4-yl)methyl)piperazin-2-yl)benzoic acid (Example 39, 0.12 g, 0.27 mmol), methylsulfonamide (51 mg, 0.54 mmol, 2.0 eq.), DMAP (66 mg, 0.54 mmol, 2.0 eq.) and EDC HCl (0.10 g, 0.54 mmol) gave enantiomer of 4-(4-(2,2-difluoroethyl)-1-((5-methoxy-7-methyl-1H-indol-4-yl)methyl)piperazin-2-yl)-N-(methylsulfonyl)benzamide (86 mg, 61%) as white airy solid. LCMS (31697 LCMS-5 C3) RT: 1.900 min; area % (215 nm): 88.3%; m/z=521.2 [M+H]⁺. ¹H-NMR (400 MHz, CD₃OD) δ 8.17 (d, J=7.8 Hz, 2H), 7.63 (d, J=7.9 Hz, 2H), 7.29 (s, 1H), 6.75 (s, 1H), 6.34 (s, 1H), 5.98 (tt, J=55.7, 4.2 Hz, 2H), 3.77 (s, 3H), 3.16 (s, 3H), 3.12-2.62 (m, 6H), 2.49 (s, 3H). ¹⁹F-NMR (376 MHz, CD₃OD) δ −121.09.

### Example 56: (R)-4-(1-((5-Methoxy-7-methyl-1H-indol-4-yl)methyl)-4-(3,3,3-trifluoropropyl)piperazin-2-yl)benzoic acid

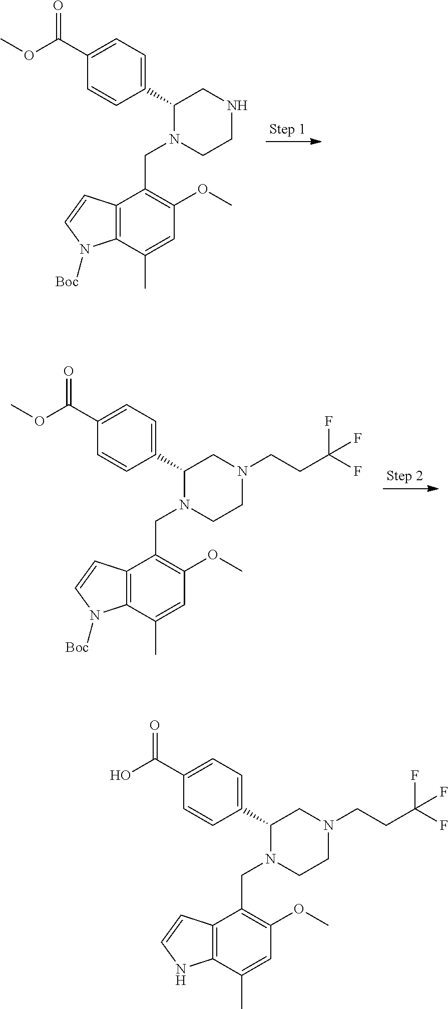

Step 1. tert-Butyl (R)-5-methoxy-4-((2-(4-(methoxycarbonyl)phenyl)-4-(3,3,3-trifluoropropyl)piperazin-1-yl)methyl)-7-methyl-1H-indole-1-carboxylate was prepared according to General Experimental Procedure 3. tert-Butyl (R)-5-methoxy-4-((2-(4-(methoxycarbonyl)phenyl)piperazin-1-yl)methyl)-7-methyl-1H-indole-1-carboxylate (130 mg, 263 μmol) and 2,2,2-trifluoropropyl trifluoromethanesulfonate (61.2 μL, 395 μmol) gave tert-butyl (R)-5-methoxy-4-((2-(4-(methoxycarbonyl)phenyl)-4-(3,3,3-trifluoropropyl)piperazin-1-yl)methyl)-7-methyl-1H-indole-1-carboxylate (150 mg, 95%) as a glassy white solid. LCMS (General 3) RT: 2.41 min; area % (254 nm): 90%; m/z=590.4 [M+H]⁺.

Step 2. (R)-4-(1-((5-Methoxy-7-methyl-1H-indol-4-yl)methyl)-4-(3,3,3-trifluoropropyl)piperazin-2-yl)benzoic acid was prepared according to General Experimental Procedure 2. (R)-5-Methoxy-4-((2-(4-(methoxycarbonyl)phenyl)-4-(3,3,3-trifluoropropyl)piperazin-1-yl)methyl)-7-methyl-1H-indole-1-carboxylate (150 mg, 254 μmol) gave (R)-4-(1-((5-methoxy-7-methyl-1H-indol-4-yl)methyl)-4-(3,3,3-trifluoropropyl)piperazin-2-yl)benzoic acid (100 mg, 83%) as a white airy solid. LCMS (31697 LCMS-5 C3) RT: 2.037 min; area % (215 nm): 99.7%; m/z=476.2 [M+H]⁺. ¹H-NMR (400 MHz, CDCl3): δ=8.11 (d, J=8.2 Hz, 2H), 7.74 (s, 2H), 7.18 (t, J=2.8 Hz, 1H), 6.67 (s, 1H), 6.57 (s, 1H), 3.77 (s, 3H), 3.60 (s, 1H), 3.41 (s, 1H), 2.90 (m, 3H), 2.81 (m, 2H), 2.64 (m, 3H), 2.44 (s, 3H), 2.31 (m, 3H). ¹⁹F-NMR (376 MHz, CDCl3): δ=−65.36.

### Example 57: (R)-4-(1-((5-Methoxy-7-methyl-1H-indol-4-yl)methyl)-4-(pyrimidin-2-yl)piperazin-2-yl)benzoic acid

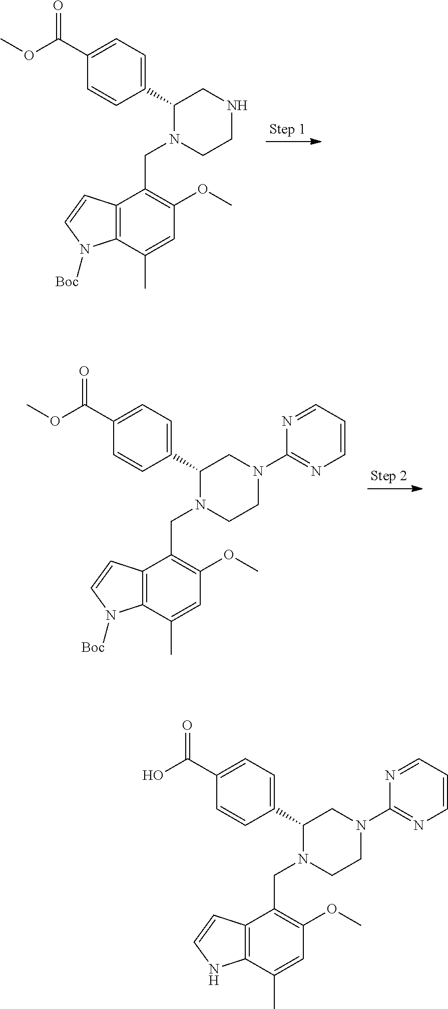

Step 1. tert-Butyl (R)-5-methoxy-4-((2-(4-(methoxycarbonyl)phenyl)-4-(pyrimidin-2-yl)piperazin-1-yl)methyl)-7-methyl-1H-indole-1-carboxylate was prepared with the following procedure. To a suspension of tert-butyl (R)-5-methoxy-4-((2-(4-(methoxycarbonyl)phenyl)piperazin-1-yl)methyl)-7-methyl-1H-indole-1-carboxylate (100 mg, 203 μmol, 1.0 eq.) and Et₃N (56.5 μL, 405 μmol, 2.0 eq.) in 1,4-dioxane (2.0 mL) in a microwave tube was added 2-bromopyrimidine (48.3 mg, 304 μmol, 1.5 eq.). The vial was flushed with N₂ and then capped and heated at 100° C. for 24 h. The reaction was worked-up; removed solvent under reduced pressure, dissolved in DMF (3 mL) and purified by automated reverse phase FCC to afford tert-butyl (R)-5-methoxy-4-((2-(4-(methoxycarbonyl)phenyl)-4-(pyrimidin-2-yl)piperazin-1-yl)methyl)-7-methyl-1H-indole-1-carboxylate (100 mg, 86%) as a white airy solid. LCMS (General 3) RT: 2.25 min; area % (254 nm): 99%; m/z=572.4 [M+H]⁺.

Step 2. (R)-4-(1-((5-Methoxy-7-methyl-1H-indol-4-yl)methyl)-4-(pyrimidin-2-yl)piperazin-2-yl)benzoic acid was prepared according to General Experimental Procedure 2. tert-Butyl (R)-5-methoxy-4-((2-(4-(methoxycarbonyl)phenyl)-4-(pyrimidin-2-yl)piperazin-1-yl)methyl)-7-methyl-1H-indole-1-carboxylate (100 mg, 175 μmol) gave (R)-4-(1-((5-methoxy-7-methyl-1H-indol-4-yl)methyl)-4-(pyrimidin-2-yl)piperazin-2-yl)benzoic acid (20 mg, 25%) as a white airy solid. LCMS (31697 LCMS-5 C3) RT: 2.023 min; area % (215 nm): 98.9%; m/z=458.2 [M+H]⁺. ¹H-NMR (400 MHz, CDCl3): δ=8.30 (d, J=4.7 Hz, 2H), 8.08 (d, J=8.1 Hz, 2H), 7.77 (d, J=8.0 Hz, 2H), 7.18 (t, J=2.8 Hz, 1H), 6.68 (s, 1H), 6.60 (s, 1H), 6.48 (t, J=4.8 Hz, 1H), 4.61 (dd, J=22.9, 13.2 Hz, 2H), 3.87 (s, 1H), 3.77 (s, 3H), 3.44 (m, 2H), 3.05 (m, 3H), 2.44 (s, 3H), 2.31 (m, 1H).

### Example 58: (R)-4-(1-((5-Methoxy-7-methyl-1H-indol-4-yl)methyl)-4-(methyl-d₃)piperazin-2-yl)benzoic acid

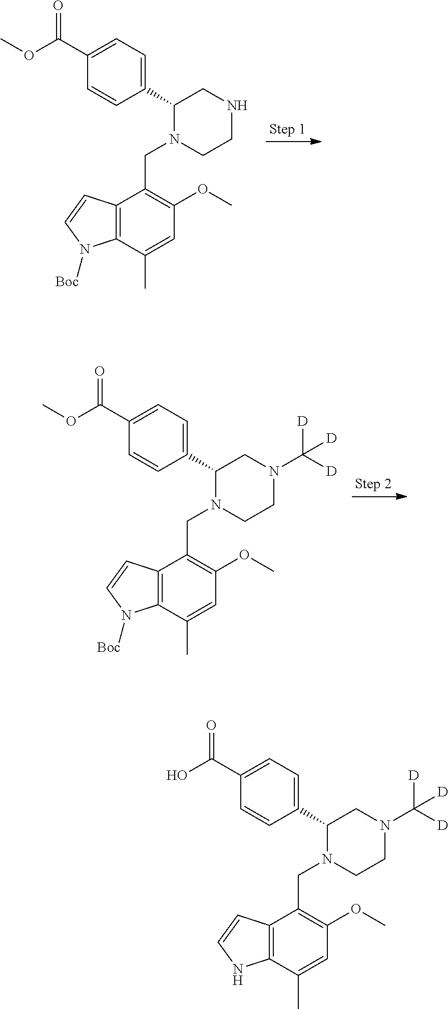

Step 1. tert-Butyl (R)-5-methoxy-4-((2-(4-(methoxycarbonyl)phenyl)-4-(methyl-d₃)piperazin-1-yl)methyl)-7-methyl-1H-indole-1-carboxylate was prepared with the following procedure. To a solution of tert-butyl (R)-5-methoxy-4-((2-(4-(methoxycarbonyl)phenyl)piperazin-1-yl)methyl)-7-methyl-1H-indole-1-carboxylate (250 mg, 506 μmol, 1.0 eq.) in CD₃OD (5 mL) was added 20 wt. % formaldehyde-d₂ in D₂O (0.5 mL, 3 mmol, 7.0 eq.). Then NaBD₄ (100 mg, 1.52 mmol, 3.0 eq.) was added and the mixture stirred at 20° C. for 15 h. The reaction was worked-up; concentrated under pressure and partitioned between CH₂Cl₂/sat. aq. NaHCO₃. The layers were separated and the aqueous layer extracted with CH₂Cl₂. The combined organic layers were washed with brine, dried over Na₂SO₄ and concentrated under reduced pressure to afford crude tert-butyl (R)-5-methoxy-4-((2-(4-(methoxycarbonyl)phenyl)-4-(methyl-d₃)piperazin-1-yl)methyl)-7-methyl-1H-indole-1-carboxylate (285 mg) as a pale yellow oil. LCMS (General 3) RT: 2.22 min; area % (254 nm): 98%; m/z=511.2 [M+H]⁺.

Step 2. (R)-4-(1-((5-Methoxy-7-methyl-1H-indol-4-yl)methyl)-4-(methyl-d₃)piperazin-2-yl)benzoic acid was prepared according to General Experimental Procedure 2. Crude tert-butyl (R)-5-methoxy-4-((2-(4-(methoxycarbonyl)phenyl)-4-(methyl-d₃)piperazin-1-yl)methyl)-7-methyl-1H-indole-1-carboxylate (285 mg) gave (R)-4-(1-((5-methoxy-7-methyl-1H-indol-4-yl)methyl)-4-(methyl-d₃)piperazin-2-yl)benzoic acid (47 mg, 21%) as a white airy solid. LCMS (31697 LCMS-5 C3) RT: 1.359 min; area % (215 nm): 98.7%; m/z=397.2 [M+H]⁺. ¹H-NMR (400 MHz, DMSO-d₆): δ 10.82 (d, J=2.4 Hz, 1H), 8.08-7.84 (m, 2H), 7.68 (d, J=7.8 Hz, 2H), 7.25 (t, J=2.8 Hz, 1H), 6.65 (s, 1H), 6.44 (dd, J=3.1, 1.9 Hz, 1H), 3.71 (s, 3H), 3.57 (d, J=11.8 Hz, 1H), 3.40 (dd, J=10.2, 3.0 Hz, 2H), 3.22 (d, J=11.8 Hz, 2H), 2.67 (ddd, J=8.7, 4.2, 2.3 Hz, 1H), 2.64-2.56 (m, 2H), 2.42 (s, 3H), 2.28-2.11 (m, 1H), 2.05-1.79 (m, 2H).

### Example 59: (R)-4-(4-(2-Hydroxyethyl)-1-((5-methoxy-7-methyl-1H-indol-4-yl)methyl)piperazin-2-yl)benzoic acid

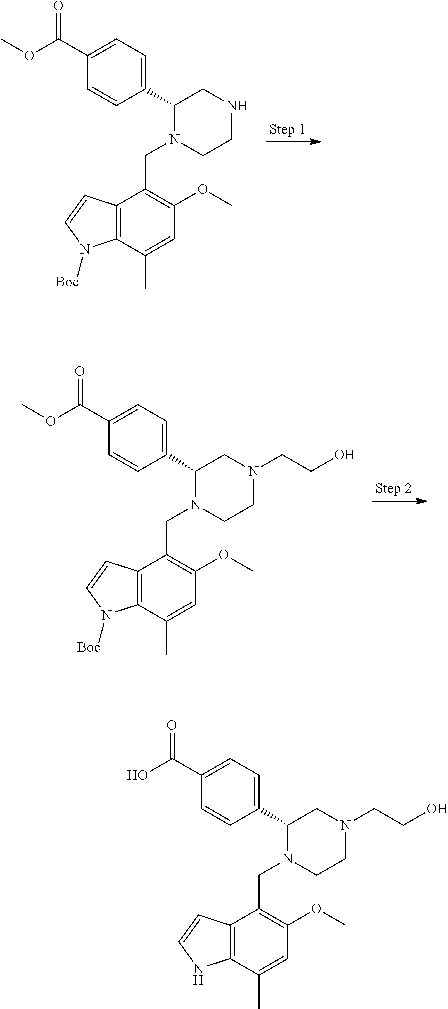

Step 1. tert-Butyl (R)-4-((4-(2-hydroxyethyl)-2-(4-(methoxycarbonyl)phenyl)piperazin-1-yl)methyl)-5-methoxy-7-methyl-1H-indole-1-carboxylate was prepared with the following procedure. To a solution of tert-butyl (R)-5-methoxy-4-((2-(4-(methoxycarbonyl)phenyl)piperazin-1-yl)methyl)-7-methyl-1H-indole-1-carboxylate (100 mg, 203 μmol, 1.0 eq.) in CH₃CN (2.5 mL) were added potassium carbonate (56.0 mg, 405 μmol, 2.0 eq.) and 2-bromoEtOAc (33.6 μL, 304 μmol, 1.5 eq.). The resulting mixture was heated at 75° C. for 15 h. LCMS indicated incomplete conversion, potassium carbonate (56.0 mg, 405 μmol, 2.0 eq.) and 2-bromoEtOAc (33.6 μL, 304 μmol, 1.5 eq.) were added and the mixture heated at 75° C. for 15 h. LCMS indicated 75% conversion, the reaction was worked-up; concentrated under reduced pressure. The residue was extracted with CH₂Cl₂, the organic layers pooled, dried over Na₂SO₄, and concentrated under reduced pressure. The residue was purified by automated FCC to afford tert-butyl (R)-4-((4-(2-hydroxyethyl)-2-(4-(methoxycarbonyl)phenyl)piperazin-1-yl)methyl)-5-methoxy-7-methyl-1H-indole-1-carboxylate (50 mg, 43%) as a pale yellow oil. LCMS (General 3) RT: 2.22 min; area % (254 nm): 93%; m/z=580.4 [M+H]⁺.

Step 2. (R)-4-(4-(2-Hydroxyethyl)-1-((5-methoxy-7-methyl-1H-indol-4-yl)methyl)piperazin-2-yl)benzoic acid was prepared according to General Experimental Procedure 2. tert-Butyl (R)-4-((4-(2-hydroxyethyl)-2-(4-(methoxycarbonyl)phenyl)piperazin-1-yl)methyl)-5-methoxy-7-methyl-1H-indole-1-carboxylate (50 mg, 86 μmol) gave (R)-4-(4-(2-hydroxyethyl)-1-((5-methoxy-7-methyl-1H-indol-4-yl)methyl)piperazin-2-yl)benzoic acid (10 mg, 28%) as a white airy solid. LCMS (31697 LCMS-5 C3) RT: 1.359 min; area % (215 nm): 99.0%; m/z=424.2 [M+H]⁺. ¹H-NMR (400 MHz, DMSO-d₆) δ 10.82 (t, J=2.3 Hz, 1H), 7.96 (d, J=8.3 Hz, 2H), 7.67 (d, J=7.8 Hz, 2H), 7.25 (t, J=2.8 Hz, 1H), 6.65 (s, 1H), 6.44 (dd, J=3.0, 1.9 Hz, 1H), 3.71 (s, 3H), 3.56 (d, J=11.9 Hz, 1H), 3.45 (t, J=6.2 Hz, 2H), 3.42-3.27 (m, 2H), 3.22 (d, J=11.8 Hz, 2H), 2.90-2.68 (m, 2H), 2.68-2.54 (m, 2H), 2.42 (s, 3H), 2.33 (td, J=6.3, 2.8 Hz, 3H), 2.21 (td, J=11.6, 2.6 Hz, 1H), 2.14-1.87 (m, 2H).

### Example 60: (R)-4-(4-(3-Fluoropropyl)-1-((5-methoxy-7-methyl-1H-indol-4-yl)methyl)piperazin-2-yl)benzoic acid

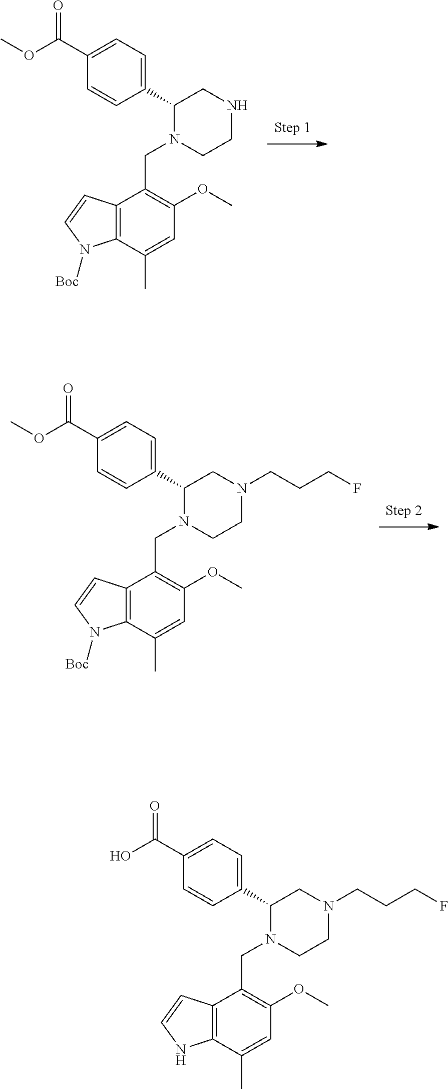

Step 1. tert-Butyl (R)-4-((4-(3-fluoropropyl)-2-(4-(methoxycarbonyl)phenyl)piperazin-1-yl)methyl)-5-methoxy-7-methyl-1H-indole-1-carboxylate was prepared according to General Experimental Procedure 3. tert-Butyl (R)-5-methoxy-4-((2-(4-(methoxycarbonyl)phenyl)piperazin-1-yl)methyl)-7-methyl-1H-indole-1-carboxylate (100 mg, 203 μmol) and 1-fluoro-3-iodopropane (31.1 μL, 305 μmol) gave tert-butyl (R)-4-((4-(3-fluoropropyl)-2-(4-(methoxycarbonyl)phenyl)piperazin-1-yl)methyl)-5-methoxy-7-methyl-1H-indole-1-carboxylate (85 mg, 76%) as a pale yellow. LCMS (General 3) RT: 2.34 min; area % (254 nm): 97%; m/z=554.3 [M+H]⁺.

Step 2. (R)-4-(4-(3-Fluoropropyl)-1-((5-methoxy-7-methyl-1H-indol-4-yl)methyl)piperazin-2-yl)benzoic acid was prepared according to General Experimental Procedure 2. tert-Butyl (R)-4-((4-(3-fluoropropyl)-2-(4-(methoxycarbonyl)phenyl)piperazin-1-yl)methyl)-5-methoxy-7-methyl-1H-indole-1-carboxylate (85 mg, 150 μmol) gave (R)-4-(4-(3-fluoropropyl)-1-((5-methoxy-7-methyl-1H-indol-4-yl)methyl)piperazin-2-yl)benzoic acid (29 mg, 43%) as a white airy solid. LCMS (31697 LCMS-5 C3) RT: 1.664 min; area % (215 nm): 98.9%; m/z=440.2 [M+H]⁺. ¹H-NMR (400 MHz, DMSO-d₆) δ 10.83 (t, J=2.3 Hz, 1H), 7.97 (d, J=8.3 Hz, 2H), 7.68 (d, J=7.8 Hz, 2H), 7.25 (t, J=2.8 Hz, 1H), 6.66 (s, 1H), 6.44 (dd, J=3.1, 1.9 Hz, 1H), 4.45 (dt, J=47.5, 6.0 Hz, 2H), 3.71 (s, 3H), 3.56 (d, J=11.9 Hz, 1H), 3.48-3.09 (m, 2H), 2.86-2.58 (m, 4H), 2.42 (s, 3H), 2.38-2.25 (m, 2H), 2.21 (td, J=11.4, 10.9, 2.4 Hz, 1H), 2.10-1.87 (m, 2H), 1.75 (dp, J=25.9, 6.6 Hz, 2H). ¹⁹F-NMR (376 MHz, DMSO-d₆) δ −218.38.

### Example 61: 4-((2R)-4-(1,1-Difluoropropan-2-yl)-1-((5-methoxy-7-methyl-1H-indol-4-yl)methyl)piperazin-2-yl)benzoic acid

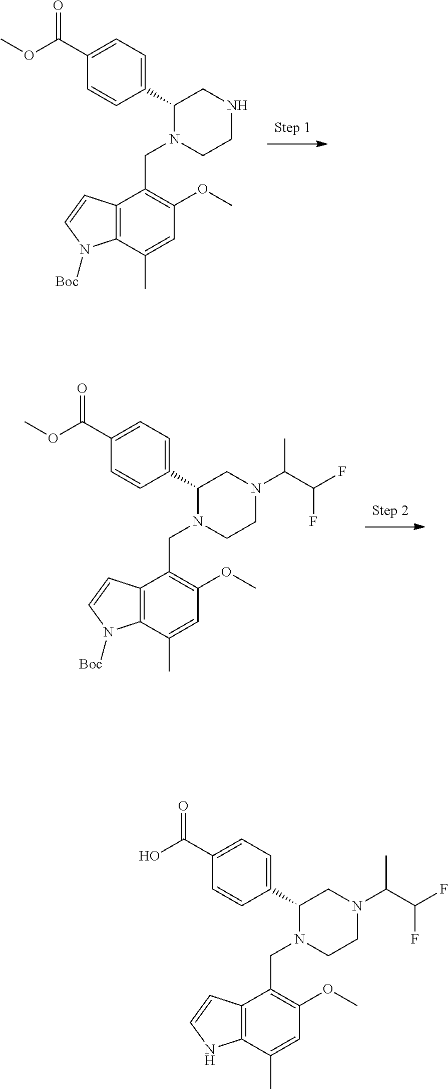

Step 1. tert-Butyl 4-(((2R)-4-(1,1-difluoropropan-2-yl)-2-(4-(methoxycarbonyl)phenyl)piperazin-1-yl)methyl)-5-methoxy-7-methyl-1H-indole-1-carboxylate was prepared according to General Experimental Procedure 1. tert-Butyl (R)-5-methoxy-4-((2-(4-(methoxycarbonyl)phenyl)piperazin-1-yl)methyl)-7-methyl-1H-indole-1-carboxylate (100 mg, 203 μmol) and 1,1-difluoropropan-2-one (65.8 μL, 812 μmol) gave crude tert-butyl 4-(((2R)-4-(1,1-difluoropropan-2-yl)-2-(4-(methoxycarbonyl)phenyl)piperazin-1-yl)methyl)-5-methoxy-7-methyl-1H-indole-1-carboxylate (120 mg). LCMS (General 3) RT: 2.40 min; area % (254 nm): 96%; m/z=572.4 [M−H]⁺.

Step 2. 4-((2R)-4-(1,1-Difluoropropan-2-yl)-1-((5-methoxy-7-methyl-1H-indol-4-yl)methyl)piperazin-2-yl)benzoic acid was prepared according to General Experimental Procedure 2. Crude tert-butyl 4-(((2R)-4-(1,1-difluoropropan-2-yl)-2-(4-(methoxycarbonyl)phenyl)piperazin-1-yl)methyl)-5-methoxy-7-methyl-1H-indole-1-carboxylate (120 mg) gave 4-((2R)-4-(1,1-difluoropropan-2-yl)-1-((5-methoxy-7-methyl-1H-indol-4-yl)methyl)piperazin-2-yl)benzoic acid (43 mg, 46% over two steps) as a airy white solid. LCMS (31697 LCMS-5 C3) RT: 1.971 and 1.985 min (1:1, diastereomeric mixture); combined area % (215 nm): >99%; m/z=458.2 [M+H]⁺. ¹H-NMR (400 MHz, DMSO-d₆) δ 10.82 (d, J=2.4 Hz, 1H), 7.97 (d, J=8.1 Hz, 2H), 7.77-7.55 (m, 2H), 7.25 (t, J=2.8 Hz, 1H), 6.65 (s, 1H), 6.43 (dt, J=3.4, 1.9 Hz, 1H), 6.01 (tdd, J=56.0, 8.3, 3.6 Hz, 1H), 3.71 (s, 3H), 3.55 (d, J=11.9 Hz, 1H), 3.48-3.09 (m, 2H), 3.01-2.84 (m, 1H), 2.84-2.57 (m, 4H), 2.42 (s, 4H), 2.18 (ddq, J=15.4, 8.3, 4.4, 4.0 Hz, 1H), 0.96 (d, J=6.8 Hz, 3H). ¹⁹F-1H coupling and diastereomers observed. ¹⁹F-NMR (376 MHz, DMSO-d₆) δ −73.40, −119.78, −119.93, −120.52, −120.68, −125.98, −126.10, −126.72, −126.85.), diastereomers observed.

### Example 62: (R)-4-(4-(3,3-Difluoropropyl)-1-((5-methoxy-7-methyl-1H-indol-4-yl)methyl)piperazin-2-yl)benzoic acid

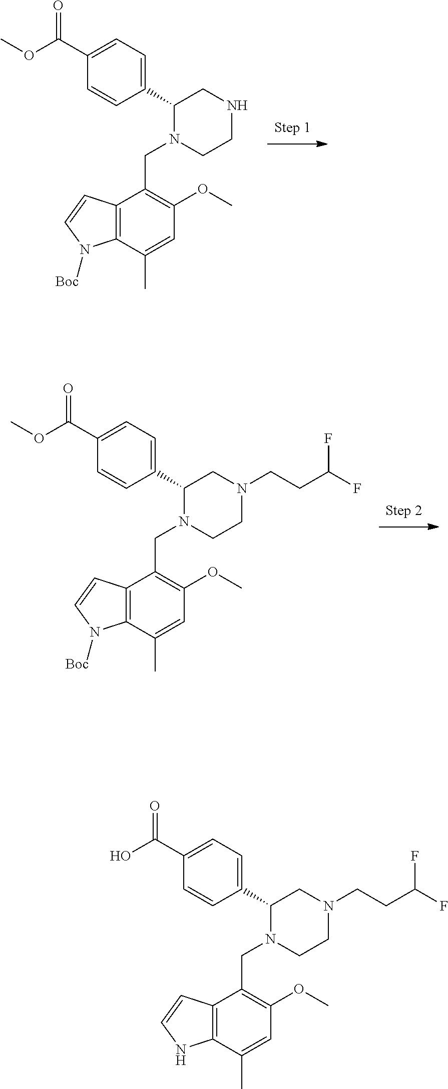

Step 1. tert-Butyl (R)-4-((4-(3,3-difluoropropyl)-2-(4-(methoxycarbonyl)phenyl)piperazin-1-yl)methyl)-5-methoxy-7-methyl-1H-indole-1-carboxylate was prepared according to General Experimental Procedure 3. tert-Butyl (R)-5-methoxy-4-((2-(4-(methoxycarbonyl)phenyl)piperazin-1-yl)methyl)-7-methyl-1H-indole-1-carboxylate (100 mg, 203 μmol) and 3,3-difluoropropyl 4-methylbenzenesulfonate (76.1 mg, 304 μmol) gave tert-butyl (R)-4-((4-(3,3-difluoropropyl)-2-(4-(methoxycarbonyl)phenyl)piperazin-1-yl)methyl)-5-methoxy-7-methyl-1H-indole-1-carboxylate (100 mg, 86%) as a glassy white solid. LCMS (General 3) RT: 2.32 min; area % (254 nm): 90%; m/z=572.3 [M+H]⁺.

Step 2. (R)-4-(4-(3,3-Difluoropropyl)-1-((5-methoxy-7-methyl-1H-indol-4-yl)methyl)piperazin-2-yl)benzoic acid was prepared according to General Experimental Procedure 2. tert-Butyl (R)-4-((4-(3,3-difluoropropyl)-2-(4-(methoxycarbonyl)phenyl)piperazin-1-yl)methyl)-5-methoxy-7-methyl-1H-indole-1-carboxylate (100 mg, 175 μmol) gave (R)-4-(4-(3,3-difluoropropyl)-1-((5-methoxy-7-methyl-1H-indol-4-yl)methyl)piperazin-2-yl)benzoic acid (75 mg, 94%) as a white airy solid. LCMS (31697 LCMS-5 C3) RT: 1.843 min; area % (215 nm): 98.9%; m/z=458.2 [M+H]⁺. ¹H-NMR (400 MHz, DMSO-d₆): δ=10.83 (s, 1H), 7.97 (d, J=8.4 Hz, 2H), 7.69 (d, J=7.8 Hz, 2H), 7.25 (t, J=2.8 Hz, 1H), 6.66 (s, 1H), 6.43 (t, J=2.5 Hz, 1H), 3.71 (s, 3H), 3.56 (d, J=11.8 Hz, 2H), 3.22 (d, J=11.8 Hz, 2H), 2.80-2.70 (m, 2H), 2.68-2.59 (m, 1H), 2.42 (s, 3H), 2.35 (dd, J=13.3, 6.3 Hz, 2H), 2.21 (m, 1H), 2.06-1.86 (m, 4H). ¹⁹F-NMR (376 MHz, DMSO-d₆): δ=−115.89.

### Example 63: (R)-4-(4-(2,2-Difluoropropyl)-1-((5-methoxy-7-methyl-1H-indol-4-yl)methyl)piperazin-2-yl)benzoic acid

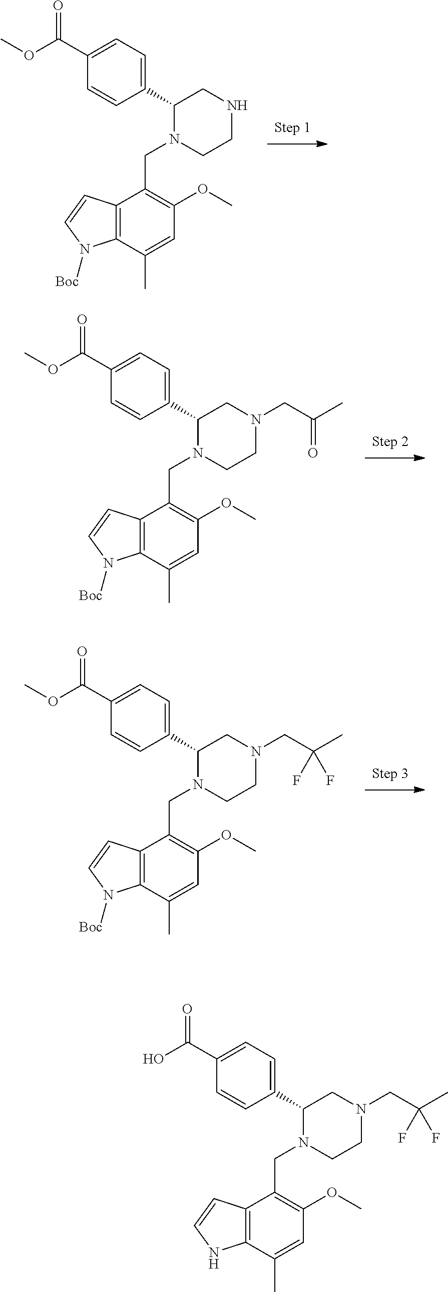

Step 1. tert-Butyl (R)-5-methoxy-4-((2-(4-(methoxycarbonyl)phenyl)-4-(2-oxopropyl)piperazin-1-yl)methyl)-7-methyl-1H-indole-1-carboxylate was prepared with the following procedure. To tert-butyl (R)-5-methoxy-4-((2-(4-(methoxycarbonyl)phenyl)piperazin-1-yl)methyl)-7-methyl-1H-indole-1-carboxylate (100 mg, 203 μmol, 1.0 eq.) and K₂CO₃ (56.0 mg, 405 μmol, 2.0 eq.) in CH₃CN (2.5 mL) was added 1-chloropropan-2-one (24.4 μL, 304 μmol, 1.5 eq.). The resulting suspension was stirred at 20° C. for 15 h. The reaction was worked-up; concentrated under reduced pressure. The residue was diluted with CH₂Cl₂/sat. aq. NaHCO₃. The layers were separated and the aqueous layer extracted with CH₂Cl₂. The combined organic layers were dried over Na₂SO₄ and concentrated under reduced pressure. The crude was purified by automated normal phase FCC to afford tert-butyl (R)-5-methoxy-4-((2-(4-(methoxycarbonyl)phenyl)-4-(2-oxopropyl)piperazin-1-yl)methyl)-7-methyl-1H-indole-1-carboxylate (60 mg, 54%). LCMS (General 3) RT: 2.15 min; area % (254 nm): 96%; m/z=550.3 [M+H]⁺.

Step 2. tert-Butyl (R)-4-((4-(2,2-difluoropropyl)-2-(4-(methoxycarbonyl)phenyl)piperazin-1-yl)methyl)-5-methoxy-7-methyl-1H-indole-1-carboxylate was prepared with the following procedure. To a solution of tert-butyl (R)-5-methoxy-4-((2-(4-(methoxycarbonyl)phenyl)-4-(2-oxopropyl)piperazin-1-yl)methyl)-7-methyl-1H-indole-1-carboxylate (60 mg, 0.11 mmol, 1.0 eq.) in CH₂Cl₂ (2.5 mL) under N₂ at −15° C. was dropwise added fresh DAST (32 μL, 0.24 mmol, 2.2 eq.). The resulting mixture was warmed to 20° C., overnight (15 h). The reaction was worked-up; cooled to 0° C. and quenched with the addition of sat. aq. NaHCO₃. The layers were separated and the aqueous layer extracted with CH₂Cl₂. The combined organic layers were dried over Na₂SO₄ and concentrated under reduced pressure. The residue was purified by automated normal phase FCC to afford tert-butyl (R)-4-((4-(2,2-difluoropropyl)-2-(4-(methoxycarbonyl)phenyl)piperazin-1-yl)methyl)-5-methoxy-7-methyl-1H-indole-1-carboxylate (15 mg, 24%). LCMS (General 3) RT: 2.32 min; area % (254 nm): 91%; m/z=572.3 [M+H]⁺.

Step 3. (R)-4-(4-(2,2-Difluoropropyl)-1-((5-methoxy-7-methyl-1H-indol-4-yl)methyl)piperazin-2-yl)benzoic acid was prepared according to General Experimental Procedure 2. tert-Butyl (R)-4-((4-(2,2-difluoropropyl)-2-(4-(methoxycarbonyl)phenyl)piperazin-1-yl)methyl)-5-methoxy-7-methyl-1H-indole-1-carboxylate (15 mg, 26 μmol) gave (R)-4-(4-(2,2-difluoropropyl)-1-((5-methoxy-7-methyl-1H-indol-4-yl)methyl)piperazin-2-yl)benzoic acid (10 mg, 83%) as a white airy solid. LCMS (31697 LCMS-5 C3) RT: 2.021 min; area % (215 nm): >99%; m/z=458.2 [M+H]⁺. ¹H-NMR (400 MHz, DMSO-d₆) δ 10.82 (t, J=2.3 Hz, 1H), 8.07-7.87 (m, 2H), 7.68 (d, J=7.8 Hz, 2H), 7.25 (t, J=2.8 Hz, 1H), 6.66 (s, 1H), 6.43 (dd, J=3.1, 1.9 Hz, 1H), 3.71 (s, 3H), 3.64-3.48 (m, 1H), 3.42 (dd, J=10.2, 2.9 Hz, 2H), 3.23 (d, J=11.8 Hz, 1H), 2.90-2.54 (m, 6H), 2.42 (s, 3H), 2.36-2.17 (m, 4H), 1.61 (t, J=19.1 Hz, 3H). ¹⁹F-NMR (DMSO-d₆) δ −90.02.

### Example 64: (R)-4-(1-((5-Methoxy-7-methyl-1H-indol-4-yl)methyl)-4-(pyridazin-3-yl)piperazin-2-yl)benzoic acid

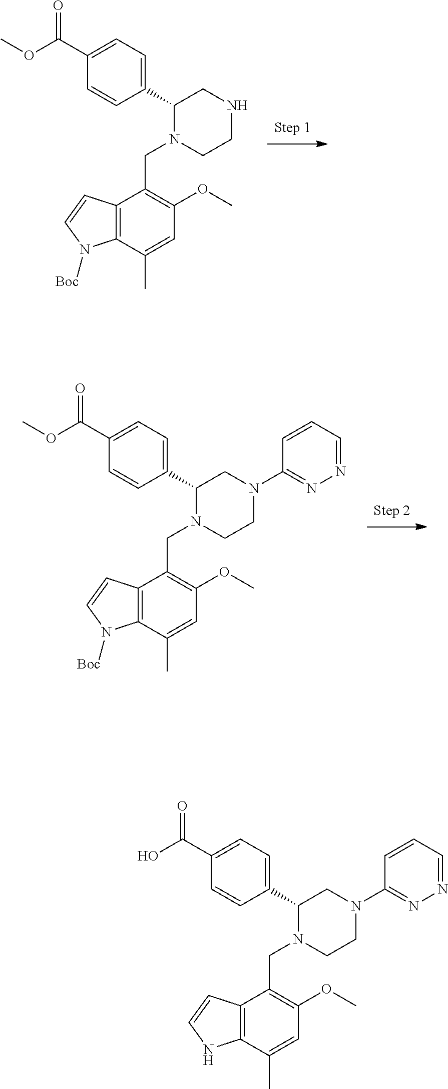

Step 1. tert-Butyl (R)-5-methoxy-4-((2-(4-(methoxycarbonyl)phenyl)-4-(pyridazin-3-yl)piperazin-1-yl)methyl)-7-methyl-1H-indole-1-carboxylate was prepared with the following procedure. tert-Butyl (R)-5-methoxy-4-((2-(4-(methoxycarbonyl)phenyl)piperazin-1-yl)methyl)-7-methyl-1H-indole-1-carboxylate (100 mg, 203 μmol, 1.0 eq.) and 3-chloropyridazine (34.8 mg, 304 μmol, 1.5 eq.) were combined in dioxane (2 mL). Et₃N (56.5 μL, 405 μmol, 2.0 eq.) was added and the resulting mixture was heated at 100° C. for 15 h. LCMS indicated 10% conversion. CuI (9.65 mg, 50.6 μmol, 0.25 eq.), Et₃N (75 μL, 538 μmol, 2.65 eq.) and 3-chloropyridazine (50 mg, 437 μmol, 2.2 eq.) were added and the mixture was heated at reflux for 15 h. The reaction was worked-up; concentrated under reduced pressure. The residue was suspended in EtOAc and water added (fine solid present). The layers were separated and the aqueous layer extracted with EtOAc. The combined organic layers were dried over Na₂SO₄ and concentrated under reduced pressure. The residue was purified by automated normal phase FCC to afford tert-butyl (R)-5-methoxy-4-((2-(4-(methoxycarbonyl)phenyl)-4-(pyridazin-3-yl)piperazin-1-yl)methyl)-7-methyl-1H-indole-1-carboxylate (25 mg, 22%) as a brown oil. LCMS (General 3) RT: 2.12 min; area % (254 nm): 95%; m/z=572.3 [M+H]⁺.

Step 2. (R)-4-(1-((5-Methoxy-7-methyl-1H-indol-4-yl)methyl)-4-(pyridazin-3-yl)piperazin-2-yl)benzoic acid was prepared according to General Experimental Procedure 2. tert-Butyl (R)-5-methoxy-4-((2-(4-(methoxycarbonyl)phenyl)-4-(pyridazin-3-yl)piperazin-1-yl)methyl)-7-methyl-1H-indole-1-carboxylate (25 mg, 44 μmol) gave (R)-4-(1-((5-methoxy-7-methyl-1H-indol-4-yl)methyl)-4-(pyridazin-3-yl)piperazin-2-yl)benzoic acid (16 mg, 79%) as a pale yellow solid. LCMS (31697 LCMS-5 C3) RT: 1.760 min; area % (215 nm): 94.9%; m/z=458.2 [M+H]⁺. ¹H-NMR (400 MHz, DMSO-d₆) δ 10.86 (t, J=2.3 Hz, 1H), 8.62-8.37 (m, 1H), 8.02 (d, J=7.7 Hz, 2H), 7.76 (d, J=7.7 Hz, 2H), 7.35 (dd, J=9.3, 4.4 Hz, 1H), 7.31-7.19 (m, 2H), 6.68 (s, 1H), 6.45 (dd, J=3.1, 1.9 Hz, 1H), 4.41-4.12 (m, 2H), 3.73 (s, 3H), 3.64 (d, J=11.7 Hz, 1H), 3.45 (dd, J=10.6, 3.2 Hz, 2H), 3.26 (d, J=11.7 Hz, 2H), 3.08-2.83 (m, 2H), 2.78 (dt, J=11.9, 2.6 Hz, 1H), 2.43 (s, 3H), 2.24 (td, J=12.0, 3.1 Hz, 1H).

### Example 65: (R)—N-(Cyclopropylsulfonyl)-4-(4-(2,2-difluoroethyl)-1-((5-methoxy-7-methyl-1H-indol-4-yl)methyl)piperazin-2-yl)benzamide

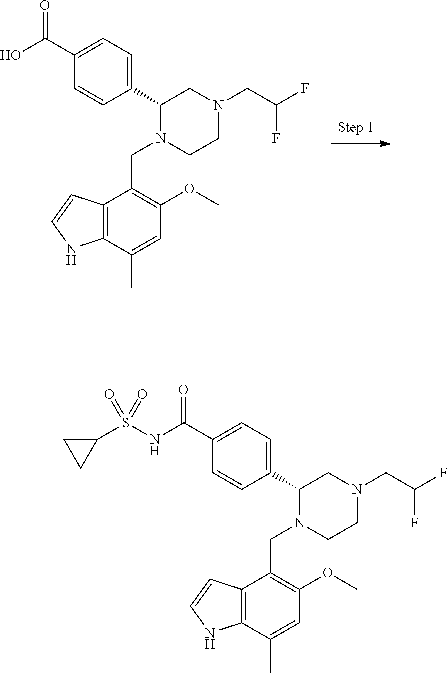 [SMILES: COc1cc(C)c2[nH]ccc2c1CN1CCN(CC(F)F)C[C@H]1c1ccc(C(=O)O)cc1 ; COc1cc(C)c2[nH]ccc2c1CN1CCN(CC(F)F)C[C@H]1c1ccc(C(=O)NS(=O)(=O)C2CC2)cc1]

Step 1. (R)—N-(Cyclopropylsulfonyl)-4-(4-(2,2-difluoroethyl)-1-((5-methoxy-7-methyl-1H-indol-4-yl)methyl)piperazin-2-yl)benzamide was prepared according to General Experimental Procedure 4. (R)-4-(4-(2,2-Difluoroethyl)-1-((5-methoxy-7-methyl-1H-indol-4-yl)methyl)piperazin-2-yl)benzoic acid (0.13 g, 0.29 mmol), cyclopropanesulfonamide (71 mg, 0.59 mmol), DMAP (72 mg, 0.59 mmol) and EDC HCl (0.11 g, 0.59 mmol) gave (R)—N-(cyclopropylsulfonyl)-4-(4-(2,2-difluoroethyl)-1-((5-methoxy-7-methyl-1H-indol-4-yl)methyl)piperazin-2-yl)benzamide (86 mg, 56%) as a white airy solid. LCMS (31697 LCMS-5 C3) RT: 2.038 min; area % (215 nm): 97.0%; m/z=547.2 [M+H]⁺. ¹H-NMR (400 MHz, CDCl₃) δ 8.15-8.07 (m, 0.5H), 8.03 (s, 1H), 7.94-7.88 (m, 2H), 7.67 (s, 1H), 7.17 (t, J=2.9 Hz, 1H), 6.66 (s, 1H), 6.56 (dd, J=3.2, 2.1 Hz, 1H), 6.50-6.43 (m, 0.5H), 5.83 (tt, J=55.9, 4.3 Hz, 1H), 3.76 (m, 4H), 3.50 (dd, J=10.3, 3.0 Hz, 2H), 3.34 (d, J=12.1 Hz, 2H), 3.15-2.99 (m, 3H), 2.81 (dt, J=8.6, 2.7 Hz, 2H), 2.78-2.61 (m, 3H), 2.47-2.42 (m, 3H), 2.41-2.27 (m, 3H), 1.38 (qd, J=5.3, 4.6, 2.4 Hz, 2H), 1.13-0.99 (m, 2H). ¹⁹F-NMR (376 MHz, CDCl₃) δ −118.46 (dtd, J=56.1, 15.0, 2.0 Hz).

### Example 66: (R)-4-(1-((5-Methoxy-7-methyl-1H-indol-4-yl)methyl)-4-(2,2,2-trifluoroethyl)piperazin-2-yl)-N-(methylsulfonyl)benzamide

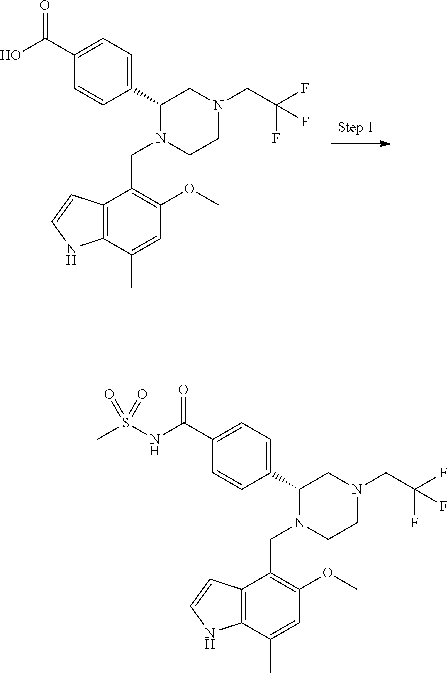 [SMILES: COc1cc(C)c2[nH]ccc2c1CN1CCN(CC(F)(F)F)C[C@H]1c1ccc(C(=O)O)cc1 ; COc1cc(C)c2[nH]ccc2c1CN1CCN(CC(F)(F)F)C[C@H]1c1ccc(C(=O)NS(C)(=O)=O)cc1]

Step 1. (R)-4-(1-((5-Methoxy-7-methyl-1H-indol-4-yl)methyl)-4-(2,2,2-trifluoroethyl)piperazin-2-yl)-N-(methylsulfonyl)benzamide was prepared according to General Experimental Procedure 4. (R)-4-(1-((5-Methoxy-7-methyl-1H-indol-4-yl)methyl)-4-(2,2,2-trifluoroethyl)piperazin-2-yl)benzoic acid (0.10 g, 0.22 mmol), methanesulfonamide (41 mg, 0.43 mmol), DMAP (53 mg, 0.43 mmol) and EDC HCl (83 mg, 0.43 mmol) gave (R)-4-(1-((5-methoxy-7-methyl-1H-indol-4-yl)methyl)-4-(2,2,2-trifluoroethyl)piperazin-2-yl)-N-(methylsulfonyl)benzamide (61 mg, 52%) as a white airy solid. LCMS (31697 LCMS-5 C3) RT: 2.108 min; area % (215 nm): 99.5%; m/z=539.2 [M+H]⁺. ¹H-NMR (400 MHz, CD₃OD) δ 8.13 (d, J=7.9 Hz, 2H), 7.60 (d, J=7.9 Hz, 2H), 7.25 (d, J=3.2 Hz, 1H), 6.70 (s, 1H), 6.31 (d, J=3.1 Hz, 1H), 4.20 (s, 1H), 3.72 (s, 3H), 3.18 (m, 3H), 3.09-2.97 (m, 4H), 3.09 (m, 2H), 2.45 (s, 3H). ¹⁹F-NMR (376 MHz, CD₃OD) δ −71.14.

### Example 67: (R)-4-(1-((5-Methoxy-7-methyl-1H-indol-4-yl)methyl)-4-(3,3,3-trifluoropropyl)piperazin-2-yl)-N-(methylsulfonyl)benzamide

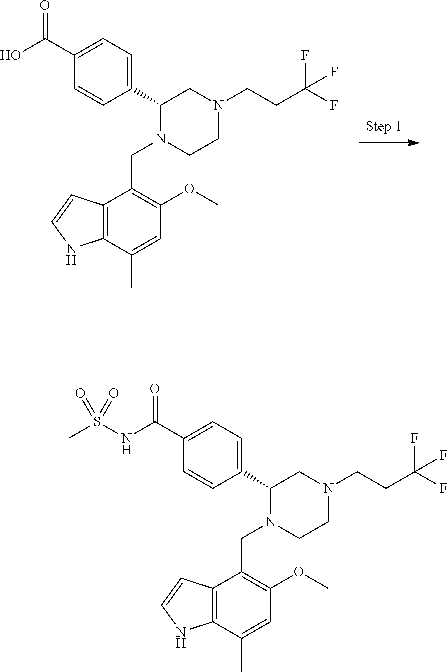 [SMILES: COc1cc(C)c2[nH]ccc2c1CN1CCN(CCC(F)(F)F)C[C@H]1c1ccc(C(=O)O)cc1 ; COc1cc(C)c2[nH]ccc2c1CN1CCN(CCC(F)(F)F)C[C@H]1c1ccc(C(=O)NS(C)(=O)=O)cc1]

Step 1. (R)-4-(1-((5-Methoxy-7-methyl-1H-indol-4-yl)methyl)-4-(3,3,3-trifluoropropyl)piperazin-2-yl)-N-(methylsulfonyl)benzamide was prepared according to General Experimental Procedure 4. (R)-4-(1-((5-Methoxy-7-methyl-1H-indol-4-yl)methyl)-4-(3,3,3-trifluoropropyl)piperazin-2-yl)benzoic acid (50 mg, 0.11 mmol), methanesulfonamide (20 mg, 0.21 mmol), DMAP (26 mg, 0.21 mmol) and EDC HCl (40 mg, 0.21 mmol) gave (R)-4-(1-((5-methoxy-7-methyl-1H-indol-4-yl)methyl)-4-(3,3,3-trifluoropropyl)piperazin-2-yl)-N-(methylsulfonyl)benzamide (16 mg, 29 μmol, 28%) as a white airy solid. LCMS (31697 LCMS-5 C3) RT: 2.066 min; area % (215 nm): 98.5%; m/z=553.2 [M+H]⁺. ¹H-NMR (400 MHz, CD₃OD) δ 8.07 (d, J=8.1 Hz, 2H), 8.02 (d, J=6.8 Hz, 1H), 7.60 (d, J=7.8 Hz, 2H), 7.22 (d, J=3.0 Hz, 1H), 6.88 (d, J=6.8 Hz, 1H), 6.68 (s, 2H), 6.33 (d, J=3.1 Hz, 1H), 3.92 (m, 2H), 3.72 (s, 3H), 3.16 (s, 1H), 3.10 (s, 3H), 3.05-2.97 (m, 1H), 2.95-2.81 (m, 3H), 2.68-2.56 (m, 3H), 2.46-2.29 (m, 7H). ¹⁹F-NMR (376 MHz, CD₃OD) δ −66.64 (t, J=10.9 Hz).

### Example 68: 4-(7-((5-methoxy-7-methyl-1H-indol-4-yl)methyl)-4-methyl-4,7-diazaspiro[2.5]octan-6-yl)benzoic acid

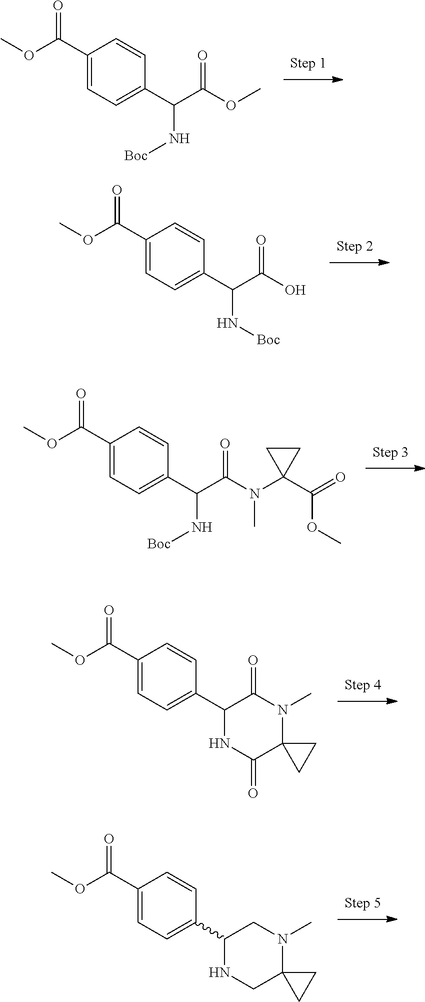

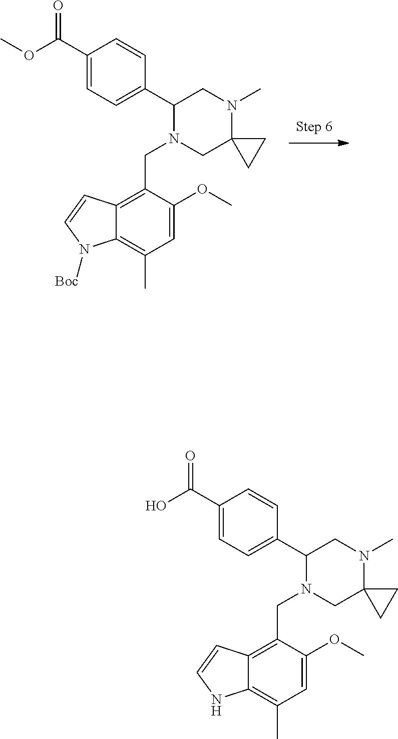 [SMILES: COC(=O)c1ccc(C2CN(C)C3(CC3)CN2Cc2c(OC)cc(C)c3c2ccn3C(=O)OC(C)(C)C)cc1 ; COc1cc(C)c2[nH]ccc2c1CN1CC2(CC2)N(C)CC1c1ccc(C(=O)O)cc1]

Step 1. Step 1. 2-((tert-Butoxycarbonyl)amino)-2-(4-(methoxycarbonyl)phenyl)acetic acid was prepared with the following procedure. To a solution of methyl 4-(1-((tert-butoxycarbonyl)amino)-2-methoxy-2-oxoethyl)benzoate (3.3 g, 1.0 eq., 10 mmol) in THE (25 mL) at 0° C. was dosed 1 M aq. LiOH (11.5 mL, 1.1 eq., 11 mmol) with a syringe pump (0.5 mL/min). The reaction was stirred at 0° C. overnight. The reaction was worked up; diluted with water (100 mL) and extracted with TBME (3×100 mL). The aqueous layer was acidified to pH 3 with 0.5 M aq. citric acid and extracted with TBME (3×100 mL). The pooled organic layers were washed with 0.5 M aq. citric acid (2×50 mL), water, brine, dried over Na₂SO₄, filtered, and concentrated under reduced pressure to afford 2-((tert-butoxycarbonyl)amino)-2-(4-(methoxycarbonyl)phenyl)acetic acid (2.7 g, 86%) as an off-white solid. LCMS (General3 AB) RT: 0.88 min; area % (254 nm): 98%; m/z=307.7 [M−H]⁻.

Step 2. Methyl 4-(1-((tert-butoxycarbonyl)amino)-2-((1-(methoxycarbonyl)cyclopropyl)(methyl)amino)-2-oxoethyl)benzoate was prepared with the following procedure. To a solution of 2-((tert-butoxycarbonyl)amino)-2-(4-(methoxycarbonyl)phenyl)acetic (1.3 g, 0.9 eq., 4.3 mmol) in DMF (50 mL) were added methyl 1-(methylamino)cyclopropane-1-carboxylate hydrochloride (0.80 g, 1 eq., 4.8 mmol) and EDC HCl (1.1 g, 1.2 eq., 5.8 mmol), upon which the solution turned light orange. Cyanic 2-(hydroxyimino)butanoic anhydride (0.82 g, 1.2 eq., 5.8 mmol) was added which turned the solution pink. Finally, Et₃N (1.3 mL, 2 eq., 9.7 mmol) was added and de reaction turned purple. The mixture was stirred at RT for 5.5 h. The reaction was worked up; diluted with 100 ml EtOAc and 100 ml heptane, washed with water (3×100 mL), NH₄Cl (2×100 mL, HCl (2×100 mL, 1.0 M), and brine (100 ml). The organic layer was dried with Na₂SO₄ and concentrated under reduced pressure. The crude product was purified by automated FCC to afford methyl 4-(1-((tert-butoxycarbonyl)amino)-2-((1-(methoxycarbonyl)cyclopropyl)(methyl)amino)-2-oxoethyl)benzoate (500 mg, 25%) as an orange oil. LCMS (General3 AB) RT: 1.56 min; area % (254 nm): 88%; m/z=443.3 [M+Na]*.

Step 3. Methyl 4-(4-methyl-5,8-dioxo-4,7-diazaspiro[2.5]octan-6-yl)benzoate was prepared with the following procedure. To a solution of methyl 4-(1-((tert-butoxycarbonyl)amino)-2-((1-(methoxycarbonyl)cyclopropyl)(methyl)amino)-2-oxoethyl)benzoate (500 mg, 1 eq., 1.19 mmol) in DCM (8 mL) was added HCl (1.78 mL, 4.0 molar in dioxane, 6.0 eq., 7.14 mmol). The resulting mixture was stirred at room temperature. After 16 h, the reaction mixture was concentrated under reduced pressure. The residue was dissolved in EtOAc (8.0 ml), NaHCO₃ (8.0 ml) was added and the mixture was stirred vigorously for 3 h. The reaction was worked up; the mixture was diluted with 100 ml EtOAc, washed with water (2×100 mL), and brine (100 ml). The organic layer was dried with Na₂SO₄ and concentrated under reduced pressure to afford methyl 4-(4-methyl-5,8-dioxo-4,7-diazaspiro[2.5]octan-6-yl)benzoate (200 mg, 58%) as light-yellow solid. LCMS (General3 AB) RT: 0.88 min; area % (254 nm): 76%; m/z=289.1 [M+H]⁺.

Step 4. Methyl 4-(4-methyl-4,7-diazaspiro[2.5]octan-6-yl)benzoate was prepared with the following procedure. To a solution of methyl 4-(4-methyl-5,8-dioxo-4,7-diazaspiro[2.5]octan-6-yl)benzoate (200 mg, 1 eq., 694 μmol) in dry THE (8 ml) was added acetylacetonato(1,5-cyclooctadiene)rhodium (21.6 mg, 0.1 eq., 70 μmol) and silylbenzene (710 μL, 8 eq., 5.34 mmol). The resulting mixture was stirred at 20° C. under N₂ atmosphere for 120 h. The reaction was worked up; the mixture was concentrated under reduced pressure and then purified by SCX-II column chromatography. The mixture was loaded on the column, eluted with MeOH (2×100 mL) followed by 7 N NH3 in MeOH (2×100 mL). The crude product was further purified by automated FCC to afford methyl 4-(4-methyl-4,7-diazaspiro[2.5]octan-6-yl)benzoate (170 mg, 94%) as a brown solid. LCMS (General3 AB) RT: 1.00 min; area % (254 nm): 88%; m/z=261.2 [M+H]⁺.

Step 5. tert-Butyl 5-methoxy-4-((6-(4-(methoxycarbonyl)phenyl)-4-methyl-4,7-diazaspiro[2.5]octan-7-yl)methyl)-7-methyl-1H-indole-1-carboxylate was prepared with the following procedure. To a solution of methyl 4-(4-methyl-4,7-diazaspiro[2.5]octan-6-yl)benzoate (170 mg, 1 eq., 653 μmol) in DCE (7 mL) was added tert-butyl 4-formyl-5-methoxy-7-methyl-1H-indole-1-carboxylate (283 mg, 1.5 eq., 979 μmol) and acetic acid (41.1 μL, 1.1 eq., 718 μmol) and the resulting mixture was stirred at room temperature. After 2 h, sodium triacetoxyborohydride (554 mg, 4.0 eq., 2.61 mmol) was added. The reaction mixture was stirred at 20° C. for 15 h. The reaction was worked up; removed volatiles under reduced pressure, diluted in EtOAc (200 ml), washed with water (100 ml), brine (100 ml), dried with Na₂SO₄, and concentrated under reduced pressure to afford the crude product tert-butyl 5-methoxy-4-((6-(4-(methoxycarbonyl)phenyl)-4-methyl-4,7-diazaspiro[2.5]octan-7-yl)methyl)-7-methyl-1H-indole-1-carboxylate as a brown solid. LCMS (General2 AB) RT: RT: 2.16 min; area % (254 nm): 19%; m/z=534.2 [M+H]⁺.

Step 6. 4-(7-((5-Methoxy-7-methyl-1H-indol-4-yl)methyl)-4-methyl-4,7-diazaspiro[2.5]octan-6-yl)benzoic acid was prepared with the following procedure. To a solution of tert-butyl 5-methoxy-4-((6-(4-(methoxycarbonyl)phenyl)-4-methyl-4,7-diazaspiro[2.5]octan-7-yl)methyl)-7-methyl-1H-indole-1-carboxylate (350 mg, 1.0 eq., 656 μmol) in MeOH (6.0 mL) was added 30 wt. % aq. sodium hydroxide (0.38 mL, 5.0 eq., 3.28 mmol) and water (800 μL), the mixture was stirred at 45° C. for 72 h. The mixture was concentrated under reduced pressure and purified by automated reverse phase FCC to afford 4-(7-((5-methoxy-7-methyl-1H-indol-4-yl)methyl)-4-methyl-4,7-diazaspiro[2.5]octan-6-yl)benzoic acid (33 mg, 12%) as a white solid. LCMS (31697 LCMS-5 C3) RT: 1.671 min; area % (215 nm): 98.6%; m/z=420.2 [M+H]⁺. ¹H-NMR (400 MHz, CD₃OD): δ=8.10 (d, J=8.5 Hz, 2H), 7.71 (d, J=7.8 Hz, 2H), 7.24 (d, J=3.1 Hz, 1H), 6.70 (s, 1H), 6.40 (d, J=3.1 Hz, 1H), 4.33 (d, J=11.3 Hz, 1H), 4.10 (d, J=12.5 Hz, 1H), 3.88 (d, J=12.4 Hz, 1H), 3.77 (s, 3H), 3.48 (d, J=12.8 Hz, 1H), 3.40-3.32 (m, 1H), 3.00 (dd, J=13.9, 3.0 Hz, 1H), 2.60 (s, 3H), 2.46 (s, 3H), 2.26 (d, J=12.8 Hz, 1H), 0.84 (m, 1H), 0.76 (m, 1H), 0.54 (m, 1H), 0.48-0.39 (m, 1H).

### Example 69: (R)-4-(1-((5-Methoxy-7-methyl-1H-indol-4-yl)methyl)-4-(thiazol-2-yl)piperazin-2-yl)benzoic acid

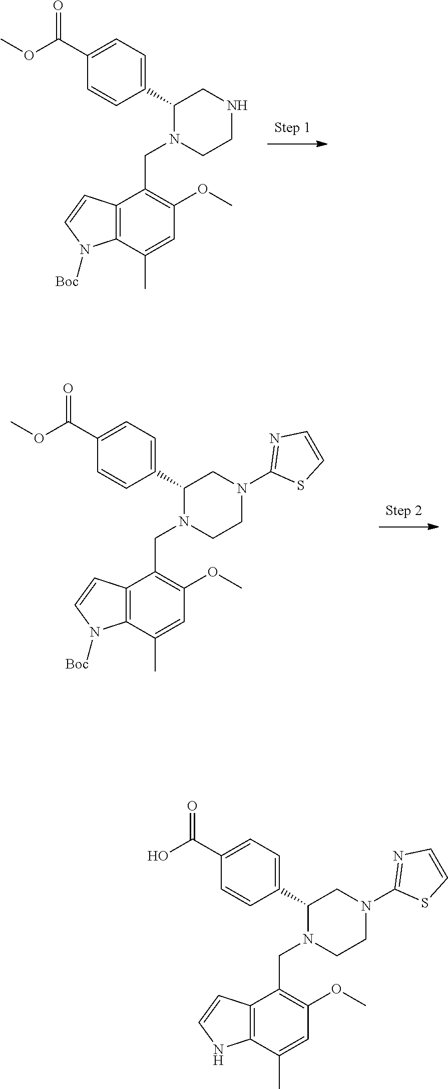

Step 1. tert-Butyl (R)-5-methoxy-4-((2-(4-(methoxycarbonyl)phenyl)-4-(thiazol-2-yl)piperazin-1-yl)methyl)-7-methyl-1H-indole-1-carboxylate was prepared with the following procedure. tert-Butyl 5-methoxy-4-((2-(4-(methoxycarbonyl)phenyl)piperazin-1-yl)methyl)-7-methyl-1H-indole-1-carboxylate (130 mg, 263 μmol, 1.0 eq.), 2-chlorothiazole (45.2 μL, 527 μmol, 2.0 eq.), CuI (12.5 mg, 65.8 μmol, 0.25 eq.), Et₃N (73.4 μL, 527 μmol, 2.0 eq.) were combined in a microwave vial and dissolved in dioxane (1.0 mL). The vial was flushed with N₂ and the resulting mixture was heated at 110° C. for 24 h. The reaction was worked-up; concentrated under reduced pressure to afford crude tert-butyl (R)-5-methoxy-4-((2-(4-(methoxycarbonyl)phenyl)-4-(thiazol-2-yl)piperazin-1-yl)methyl)-7-methyl-1H-indole-1-carboxylate (150 mg). LCMS (General 3) RT: 2.41 min; area % (254 nm): 58%; m/z=577.3 [M+H]⁺.

Step 2. (R)-4-(1-((5-Methoxy-7-methyl-1H-indol-4-yl)methyl)-4-(thiazol-2-yl)piperazin-2-yl)benzoic acid was prepared according General Experimental Procedure 2. Crude tert-butyl (R)-5-methoxy-4-((2-(4-(methoxycarbonyl)phenyl)-4-(thiazol-2-yl)piperazin-1-yl)methyl)-7-methyl-1H-indole-1-carboxylate (150 mg) gave (R)-4-(1-((5-methoxy-7-methyl-1H-indol-4-yl)methyl)-4-(thiazol-2-yl)piperazin-2-yl)benzoic acid (5 mg, 4%) as a white airy solid. LCMS (31697 LCMS-5 C3) RT: 2.088 min; area % (215 nm): 95.3%; m/z=463.2 [M+H]⁺. ¹H-NMR (400 MHz, CD₃OD): δ=8.07 (d, J=7.9 Hz, 2H), 7.71 (d, J=7.8 Hz, 2H), 7.19 (d, J=3.1 Hz, 1H), 7.08 (d, J=3.7 Hz, 1H), 6.70 (d, J=3.7 Hz, 1H), 6.67 (s, 1H), 6.42 (d, J=3.1 Hz, 1H), 3.84 (m, 2H), 3.75 (s, 3H), 3.72 (m, 1H), 3.66-3.59 (m, 1H), 3.37 (d, J=11.9 Hz, 1H), 3.22-3.10 (m, 2H), 3.01-2.95 (m, 1H), 2.43 (s, 3H).

### Example 70: tert-Butyl 5-methoxy-4-((2-(6-(methoxycarbonyl)pyridin-3-yl)-4-methylpiperazin-1-yl)methyl)-7-methyl-1H-indole-1-carboxylate

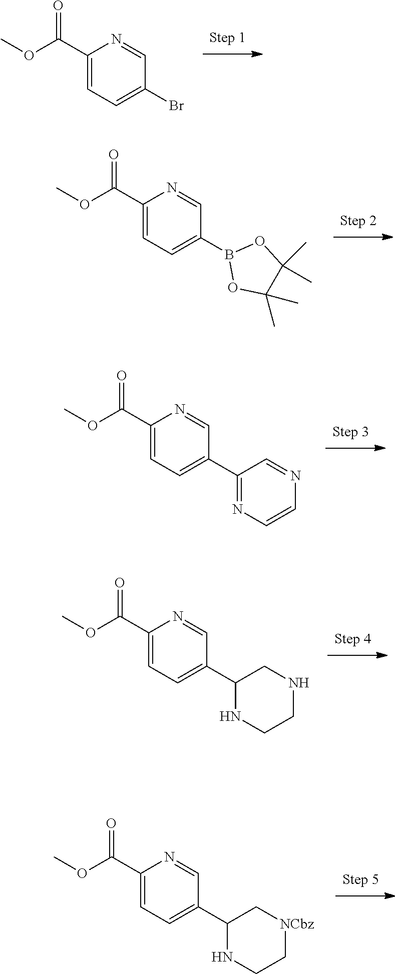

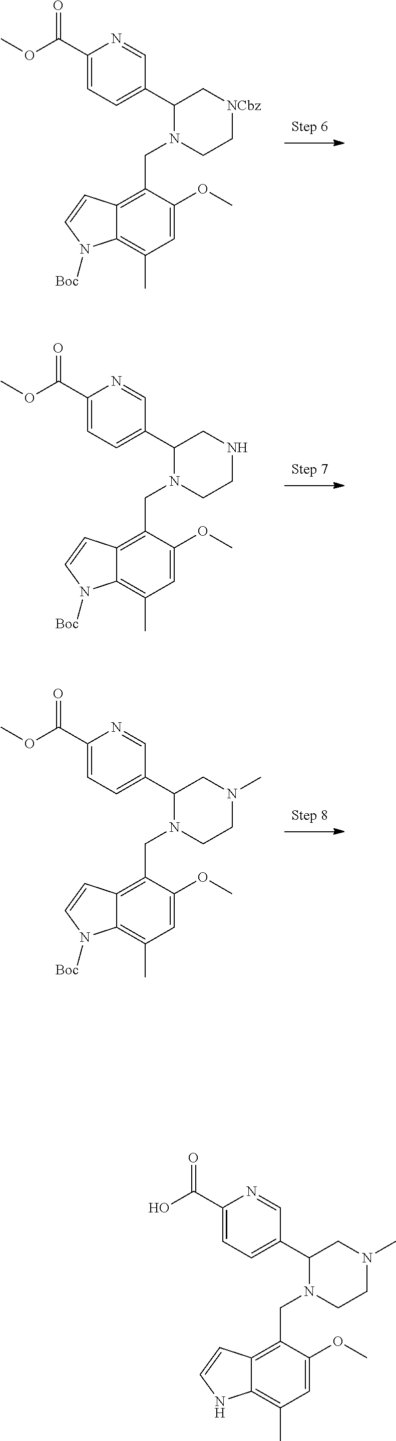

Step 1. Methyl 5-(4,4,5,5-tetramethyl-1,3,2-dioxaborolan-2-yl)picolinate was prepared with the following procedure. Methyl 5-Bromopicolinate (20.00 g, 1.0 eq, 92.58 mmol) was dissolved in 1,4-dioxane (300 mL). Then bis(pinacolato)diboron (23.51 g, 1.0 eq, 92.58 mmol), PdCl₂(dppf) CH₂Cl₂ (3.78 g, 0.05 eq, 4.63 mmol) and Pd(OAc)₂ (27.26 g, 3.0 eq, 277.70 mmol) were added. The reaction mixture was degassed for 10 minutes with N₂ and then stirred at 90° C. for 3 hours. The reaction was worked-up; cooled to room temperature and filtered over Celite. The filter cake was washed with EtOAc. The filtrate was concentrated under reduced pressure and purified with FCC to afford methyl 5-(4,4,5,5-tetramethyl-1,3,2-dioxaborolan-2-yl)picolinate (20.0 g, 82%) as white. LCMS (General 4) RT: 1.01 min; area % (254 nm): 97%; m/z=264.2 [M+H]⁺.

Step 2. Methyl 5-(pyrazin-2-yl)picolinate was prepared with the following procedure. Methyl 5-(4,4,5,5-tetramethyl-1,3,2-dioxaborolan-2-yl)picolinate (16.55 g, 1.0 eq, 62.90 mmol) was dissolved in DMSO (160 mL). Then 2-chloropyrazine (7.20 g, 5.63 mL, 1.0 eq, 62.90 mmol), PdCl₂(dppf) CH₂Cl₂ (1.54 g, 0.03 eq, 1.89 mmol), K₂CO₃ (9.56 g, 1.1 eq, 69.19 mmol) were added. The reaction mixture was degassed for 10 min with N₂ and stirred at 90° C. for 15 h. The reaction was worked-up; cooled to room temperature and filtered over Celite. The filter cake was washed with DCM (250 mL). The filtered was washed with H₂O (250 mL) and, after separating the layers, the aqueous layer extracted with DCM (2×200 mL). The combined organic layers were dried over Na₂SO₄ and concentrated under reduced pressure. The residue was purified by normal phase FCC to afford After concentration, the residue was purified with FCC to afford methyl 5-(pyrazin-2-yl)picolinate (6.8 g, 50%) as brown solid. LCMS (General 3) RT: 0.71 min; area % (254 nm): 85%; m/z=216.1 [M+H]⁺.

Step 3. Methyl 5-(piperazin-2-yl)picolinate was prepared with the following procedure. Methyl 5-(pyrazin-2-yl)picolinate (6.8 g, 1.0 eq, 31.7 mmol) was suspended in EtOAc (150 mL). Then 10% Pd/C (3.37 g, 0.1 eq, 3.17 mmol) was added and the resulting reaction mixture was stirred under H₂ pressure (balloon) at 65° C. for 72 h. The reaction was worked-up; cooled to room temperature and filtered over Celite. The filter cake was washed with EtOAc (100 mL), DCM (100 mL) and MeOH (100 mL). The filtrate was concentrated under reduced pressure to afford crude methyl 5-(piperazin-2-yl)picolinate (6.2 g, 88%) as brown oil. ¹H-NMR (400 MHz, CDCl₃) δ 8.71 (s, 1H), 8.07 (d, J=8.0 Hz, 1H), 7.88 (dd, J=8.1, 2.2 Hz, 1H), 3.97 (s, 3H), 3.91 (dd, J=10.3, 2.8 Hz, 1H), 3.13-2.85 (m, 5H), 2.67 (dd, J=12.0, 10.2 Hz, 1H), 2.31-2.20 (br. s, 2H).

Step 4. Benzyl 3-(6-(methoxycarbonyl)pyridin-3-yl)piperazine-1-carboxylate was prepared with the following procedure. Methyl 5-(piperazin-2-yl)picolinate (350 mg, 1.0 eq, 1.58 mmol) and DIPEA (551 μL, 2.0 eq, 3.16 mmol) were dissolved in MeCN (10 mL) and cooled to 0° C. Then benzyl chloroformate (226 μL, 1.0 eq, 1.58 mmol) was added dropwise. The resulting mixture was stirred at 0° C. for 2 hours. The reaction was worked-up; quenched with the addition of H₂O and removed MeCN under reduced pressure. DCM (20 mL) was added to the residue and the layers separated. The organic layer washed with sat. aq. NaHCO₃ (15 mL), dried over Na₂SO₄, and concentrated under reduced pressure. The residue was purified with FCC to afford benzyl 3-(6-(methoxycarbonyl)pyridin-3-yl)piperazine-1-carboxylate (390 mg, 69%) as clear oil. LCMS (General 3) RT: 1.24 min; area % (254 nm): 100%; m/z=356.1 [M+H]⁺.

Step 5. tert-Butyl 4-((4-((benzyloxy)carbonyl)-2-(6-(methoxycarbonyl)pyridin-3-yl)piperazin-1-yl)methyl)-5-methoxy-7-methyl-1H-indole-1-carboxylate was prepared with the following procedure. Benzyl 3-(6-(methoxycarbonyl)pyridin-3-yl)piperazine-1-carboxylate (370 mg, 1.0 eq, 1.04 mmol), tert-butyl 4-formyl-5-methoxy-7-methyl-1H-indole-1-carboxylate (602 mg, 2.0 eq, 2.08 mmol), and AcOH(68.8 mg, 65.6 μL, 1.1 eq, 1.15 mmol) were dissolved in 1,2-dichloroethane (10 mL) and stirred at r.t. for 30 min. Then NaBH(OAc)₃ (883 mg, 4.0 eq, 4.16 mmol) was added and the mixture stirred at 20° C. for 15 h. DCM (15 mL) was added and the organic layer washed with sat. aq. NaHCO₃ (20 mL). The organic layer was dried over Na₂SO₄ and concentrated under reduced pressure. The residue was purified with FCC to afford tert-butyl 4-((4-((benzyloxy)carbonyl)-2-(6-(methoxycarbonyl)pyridin-3-yl)piperazin-1-yl)methyl)-5-methoxy-7-methyl-1H-indole-1-carboxylate (571 mg, 87%) as white foam. LCMS (General 3) RT: 2.19 min; area % (254 nm): 100%; m/z=629.3 [M+H]⁺.

Step 6 and 7. tert-Butyl 5-methoxy-4-((2-(6-(methoxycarbonyl)pyridin-3-yl)-4-methylpiperazin-1-yl)methyl)-7-methyl-1H-indole-1-carboxylate was prepared with the following procedure. tert-Butyl 4-((4-((benzyloxy)carbonyl)-2-(6-(methoxycarbonyl)pyridin-3-yl)piperazin-1-yl)methyl)-5-methoxy-7-methyl-1H-indole-1-carboxylate (250 mg, 1.0 eq, 398 μmol) was dissolved in MeOH (6 mL) and H₂O (500 μL). Then 10% Pd/C (127 mg, 0.3 eq, 119 μmol) was added and the reaction mixture stirred under H₂ pressure (balloon) at 20° C. overnight. Then 37 wt. % aq. formaldehyde (13.7 μL, 0.5 eq, 199 μmol) was added and the resulting mixture was stirred at 45° C. for 4 hours. The reaction was worked-up; cooled to room temperature and filtered over Celite. The filter cake was rinsed with DCM (2×10 mL) and the filtrate was concentrated under reduced pressure. The residue was purified by FCC to afford tert-butyl 5-methoxy-4-((2-(6-(methoxycarbonyl)pyridin-3-yl)-4-methylpiperazin-1-yl)methyl)-7-methyl-1H-indole-1-carboxylate (128 mg, 63% over two steps) as white solid. LCMS (General 3) RT: 1.87 min; area % (254 nm): 97%; m/z=509.3 [M+H]⁺.

Step 8. tert-Butyl 5-methoxy-4-((2-(6-(methoxycarbonyl)pyridin-3-yl)-4-methylpiperazin-1-yl)methyl)-7-methyl-1H-indole-1-carboxylate was prepared according to General Experimental Procedure 2. tert-Butyl 5-methoxy-4-((2-(6-(methoxycarbonyl)pyridin-3-yl)-4-methylpiperazin-1-yl)methyl)-7-methyl-1H-indole-1-carboxylate (128 mg, 1.0 eq, 252 μmol) gave tert-butyl 5-methoxy-4-((2-(6-(methoxycarbonyl)pyridin-3-yl)-4-methylpiperazin-1-yl)methyl)-7-methyl-1H-indole-1-carboxylate (39 mg, 39%) as a white airy solid. LCMS (31697B TFA LCMS-5 C3) RT: 1.316 min; area % (220 nm): 98.3%; m/z=395.2 [M+H]⁺. ¹H-NMR (400 MHz, CD₃OD) δ 8.75 (s, 1H), 8.12 (s, 2H), 7.19 (d, J=3.1 Hz, 1H), 6.64 (s, 1H), 6.37 (d, J=3.1 Hz, 1H), 3.88-3.68 (m, 5H), 3.43 (d, J=12.0 Hz, 1H), 3.28-3.20 (m, 2H), 3.05 (dd, J=12.9, 2.7 Hz, 1H), 2.95-2.78 (m, 2H), 2.68-2.60 (m, 4H), 2.44 (s, 3H).

### Example 71: 4-((2R)-1-((5-Methoxy-7-methyl-1H-indol-4-yl)methyl)-4-(4,4,4-trifluorobutan-2-yl)piperazin-2-yl)benzoic acid

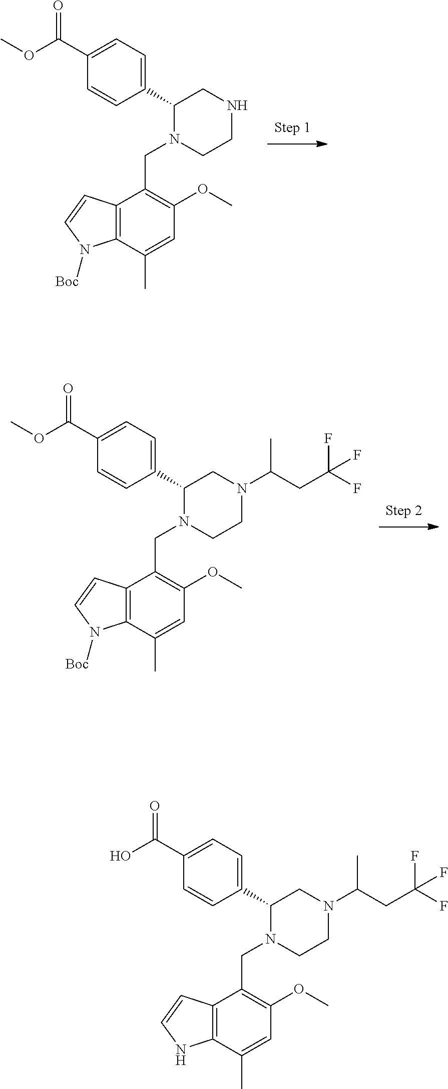

Step 1. tert-Butyl 5-methoxy-4-(((2R)-2-(4-(methoxycarbonyl)phenyl)-4-(4,4,4-trifluorobutan-2-yl)piperazin-1-yl)methyl)-7-methyl-1H-indole-1-carboxylate was prepared according to General Experimental Procedure 1. tert-Butyl (R)-5-methoxy-4-((2-(4-(methoxycarbonyl)phenyl)piperazin-1-yl)methyl)-7-methyl-1H-indole-1-carboxylate (80 mg, 0.16 mmol, 1.0 eq.) and 4,4,4-trifluoro-2-butanone (74 μL, 0.64 mmol, 4.0 eq.) gave crude tert-butyl 5-methoxy-4-(((2R)-2-(4-(methoxycarbonyl)phenyl)-4-(4,4,4-trifluorobutan-2-yl)piperazin-1-yl)methyl)-7-methyl-1H-indole-1-carboxylate (100 mg) as a pale yellow oil. LCMS (General 3) RT: 2.39 min; area % (254 nm): 83%; m/z=604.3 [M+H]⁺.

Step 2. 4-((2R)-1-((5-methoxy-7-methyl-1H-indol-4-yl)methyl)-4-(4,4,4-trifluorobutan-2-yl)piperazin-2-yl)benzoic acid was prepared according to General Experimental Procedure 2. Crude tert-butyl 5-methoxy-4-(((2R)-2-(4-(methoxycarbonyl)phenyl)-4-(4,4,4-trifluorobutan-2-yl)piperazin-1-yl)methyl)-7-methyl-1H-indole-1-carboxylate (100 mg) gave 4-((2R)-1-((5-methoxy-7-methyl-1H-indol-4-yl)methyl)-4-(4,4,4-trifluorobutan-2-yl)piperazin-2-yl)benzoic acid (45 mg, 52% over two steps) as a white airy solid. LCMS (31697 LCMS-5 C3) RT: 1.917 min; area % (215 nm): 94.0%; m/z=489.2 [M+H]⁺. ¹H-NMR (400 MHz, CD₃OD): δ =8.05-7.98 (d, J=8.43 Hz, 2H), 7.59 (d, J=7.7 Hz, 2H), 7.18 (d, J=3.1 Hz, 1H), 6.68 (s, 1H), 6.42 (dd, J=3.1, 0.8 Hz, 1H), 3.80 (d, J=11.9 Hz, 1H), 3.76 (s, 3H), 3.47-3.39 (m, 1H), 3.27 (d, J=11.8 Hz, 1H), 2.97 (m, 1H), 2.93-2.87 (m, 1H), 2.73-2.56 (m, 2H), 2.49 (m, 2H), 2.45 (s, 3H), 2.44-2.34 (m, 2H), 2.11 (m, 1H), 1.11 (d, J=6.6 Hz, 3H). ¹⁹F-NMR (376 MHz, CD₃OD): δ=−65.03.

### Example 72: tert-Butyl 5-methoxy-4-((2-(6-(methoxycarbonyl)pyridin-3-yl)-4-(3,3,3-trifluoropropyl)piperazin-1-yl)methyl)-7-methyl-1H-indole-1-carboxylate

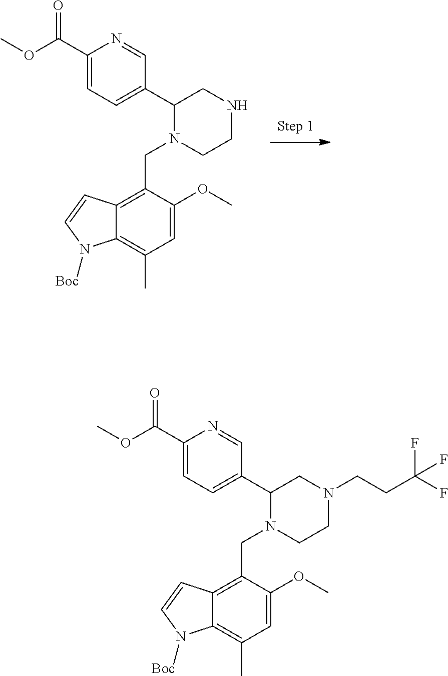

Step 1. tert-Butyl 5-methoxy-4-((2-(6-(methoxycarbonyl)pyridin-3-yl)-4-(3,3,3-trifluoropropyl)piperazin-1-yl)methyl)-7-methyl-1H-indole-1-carboxylate was prepared with the following procedure. tert-Butyl 5-methoxy-4-((2-(6-(methoxycarbonyl)pyridin-3-yl)piperazin-1-yl)methyl)-7-methyl-1H-indole-1-carboxylate (730 mg, 1.48 mmol, 1.0 eq.) was dissolved in THE (5 mL). Then Et₃N (370 μL, 2.66 mmol, 1.8 eq.) and a solution of 3,3,3-trifluoropropyl trifluoromethanesulfonate (363 mg, 1.48 mmol, 1.0 eq.) in THE (3 mL) were added. The reaction mixture was stirred at 20° C. for 15 h. The reaction was worked-up; concentrated under reduced pressure and diluted the residue with DCM. The organic layer was washed with sat. aq. NH₄Cl, dried over Na₂SO₄, and concentrated under reduced pressure. The crude was purified by automated normal phase FCC to afford tert-butyl 5-methoxy-4-((2-(6-(methoxycarbonyl)pyridin-3-yl)-4-(3,3,3-trifluoropropyl)piperazin-1-yl)methyl)-7-methyl-1H-indole-1-carboxylate (600 mg, 69) as a light yellow solid. LCMS (General 3) RT: 2.14 min; area % (254 nm): 97%; m/z=591.3 [M+H]⁺. ¹H-NMR (400 MHz, CDCl₃) δ 8.85 (s, 1H), 8.12 (d, J=8.0 Hz, 1H), 8.03 (d, J=8.2 Hz, 1H), 7.48 (d, J=3.8 Hz, 1H), 6.64 (s, 1H), 6.54 (d, J=3.8 Hz, 1H), 4.02 (s, 3H), 3.78 (s, 3H), 3.65 (d, J=12.2 Hz, 1H), 3.53 (d, J=9.9 Hz, 1H), 3.34 (d, J=12.3 Hz, 1H), 2.92-2.64 (m, 4H), 2.58 (s, 3H), 2.55 (s, 1H), 2.46-2.09 (m, 6H), 1.62 (s, 9H), 1.60 (s, 1H), 1.34-1.09 (m, 2H), 0.95-0.74 (m, 2H).

### Example 73: Enantiomer 1 of 5-(1-((5-methoxy-7-methyl-1H-indol-4-yl)methyl)-4-(3,3,3-trifluoropropyl)piperazin-2-yl)picolinic acid

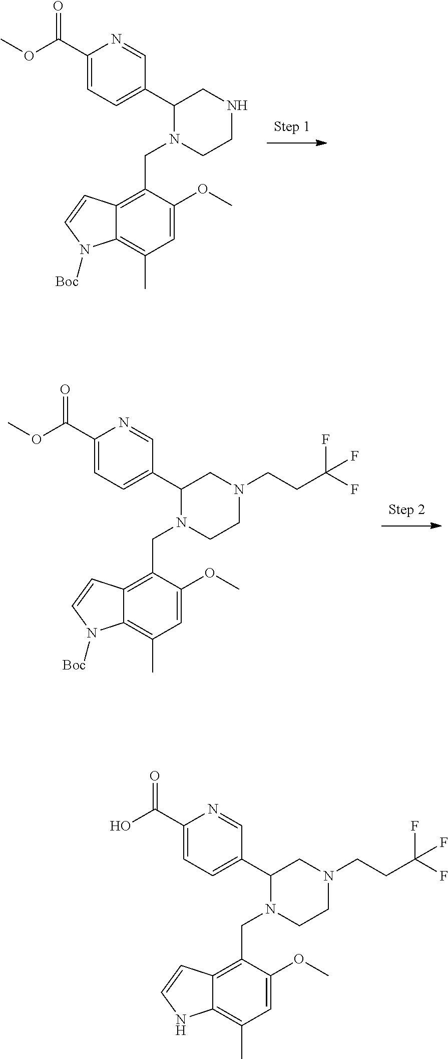

Step 1. Enantiomer 1 of tert-butyl 5-methoxy-4-((2-(6-(methoxycarbonyl)pyridin-3-yl)-4-(3,3,3-trifluoropropyl)piperazin-1-yl)methyl)-7-methyl-1H-indole-1-carboxylate was prepared with the following procedure. Racemic tert-butyl 5-methoxy-4-((2-(6-(methoxycarbonyl)pyridin-3-yl)-4-(3,3,3-trifluoropropyl)piperazin-1-yl)methyl)-7-methyl-1H-indole-1-carboxylate (500 mg) was separated by preparative chiral HPLC (31697-2101B-prep) to obtain enantiomer 1 of tert-butyl 5-methoxy-4-((2-(6-(methoxycarbonyl)pyridin-3-yl)-4-(3,3,3-trifluoropropyl)piperazin-1-yl)methyl)-7-methyl-1H-indole-1-carboxylate (180 mg). First eluting enantiomer (31679Q UPC² 4m Cel4) RT=2.841 min, 97.4% ee, m/z=591.2 [M+H]⁺.

Step 2. Enantiomer 1 of 5-(1-((5-methoxy-7-methyl-1H-indol-4-yl)methyl)-4-(3,3,3-trifluoropropyl)piperazin-2-yl)picolinic acid was prepared according to General Experimental Procedure 2. Enantiomer 1 of tert-butyl 5-methoxy-4-((2-(6-(methoxycarbonyl)pyridin-3-yl)-4-(3,3,3-trifluoropropyl)piperazin-1-yl)methyl)-7-methyl-1H-indole-1-carboxylate (180 mg) gave enantiomer 1 of 5-(1-((5-methoxy-7-methyl-1H-indol-4-yl)methyl)-4-(3,3,3-trifluoropropyl)piperazin-2-yl)picolinic acid (78 mg, 54%) as a white airy solid. LCMS (31697S TFA LCMS-5 C1) RT: 1.881 min; area % (220 nm): 98.7%; m/z=477.2 [M+H]⁺. ¹H-NMR (400 MHz, CD₃OD) δ 8.75 (s, 1H), 8.11 (s, 2H), 7.25 (d, J=3.1 Hz, 1H), 6.66 (s, 1H), 6.40 (d, J=3.2 Hz, 1H), 4.02 (d, J=10.5 Hz, 1H), 3.96 (d, J=12.2 Hz, 1H), 3.74 (s, 3H), 3.70 (d, J=12.3 Hz, 1H), 3.17 (d, J=12.2 Hz, 1H), 3.00 (dd, J=22.4, 11.9 Hz, 2H), 2.86 (t, J=11.9 Hz, 1H), 2.77-2.64 (m, 2H), 2.51-2.36 (m, 7H). ¹⁹F-NMR (376 MHz, CD₃OD) δ −66.89.

### Example 74: Enantiomer 2 of 5-(1-((5-methoxy-7-methyl-1H-indol-4-yl)methyl)-4-(3,3,3-trifluoropropyl)piperazin-2-yl)picolinic acid

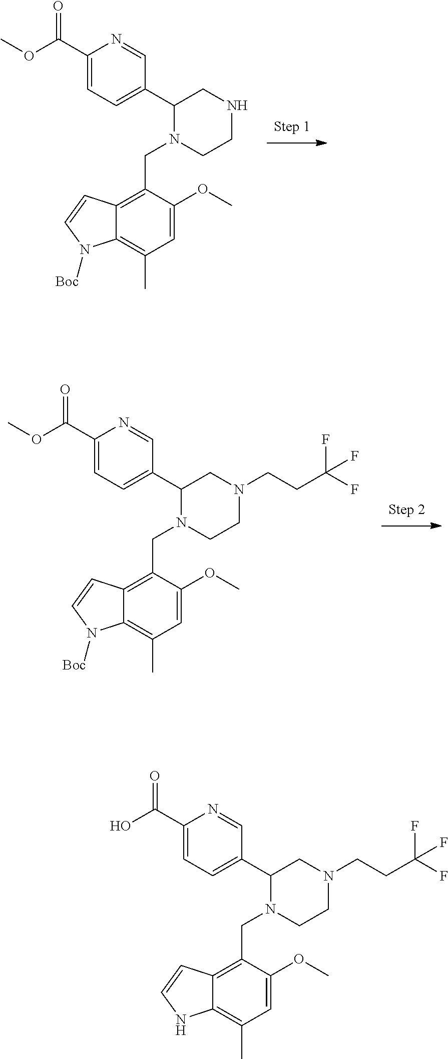

Step 1. Enantiomer 2 of tert-butyl 5-methoxy-4-((2-(6-(methoxycarbonyl)pyridin-3-yl)-4-(3,3,3-trifluoropropyl)piperazin-1-yl)methyl)-7-methyl-1H-indole-1-carboxylate was prepared with the following procedure. Racemic tert-butyl 5-methoxy-4-((2-(6-(methoxycarbonyl)pyridin-3-yl)-4-(3,3,3-trifluoropropyl)piperazin-1-yl)methyl)-7-methyl-1H-indole-1-carboxylate (500 mg) was separated by preparative chiral HPLC (31697-2101B-prep) to obtain enantiomer 2 of tert-butyl 5-methoxy-4-((2-(6-(methoxycarbonyl)pyridin-3-yl)-4-(3,3,3-trifluoropropyl)piperazin-1-yl)methyl)-7-methyl-1H-indole-1-carboxylate (170 mg). Second eluting enantiomer (31679Q UPC² 4m Cel4) RT=3.117 min, 96.7% ee, m/z=591.2 [M+H]⁺.

Step 2. Enantiomer 2 of 5-(1-((5-methoxy-7-methyl-1H-indol-4-yl)methyl)-4-(3,3,3-trifluoropropyl)piperazin-2-yl)picolinic acid was prepared according to General Experimental Procedure 2. Enantiomer 2 of tert-butyl 5-methoxy-4-((2-(6-(methoxycarbonyl)pyridin-3-yl)-4-(3,3,3-trifluoropropyl)piperazin-1-yl)methyl)-7-methyl-1H-indole-1-carboxylate (170 mg) gave enantiomer 2 of 5-(1-((5-methoxy-7-methyl-1H-indol-4-yl)methyl)-4-(3,3,3-trifluoropropyl)piperazin-2-yl)picolinic acid (71 mg, 52%) as a white airy solid. LCMS (31697S TFA LCMS-5 C1) RT: 1.881 min; area % (220 nm): 98.5%; m/z=477.2 [M+H]⁺. ¹H-NMR and ¹⁹F-NMR were identical to enantiomer 1 of 5-(1-((5-methoxy-7-methyl-1H-indol-4-yl)methyl)-4-(3,3,3-trifluoropropyl)piperazin-2-yl)picolinic acid.

### Example 75: Provided Compounds can Inhibit Complement Activation

Among other things, the present disclosure provides compounds and compositions that can modulate complement activation. In some embodiments, provided technologies inhibit complement activation. In some embodiments, provided technologies inhibit C3 convertase. Various technologies are available for assessing provided compounds, compositions and methods. In some embodiments, a useful assay is an assay described below. In some embodiments, IC50 of a provided compound measured in an assay is about or no more than about 1000, 900, 800, 700, 600, 500, 400, 300, 200, 100, 90, 80, 70, 60, 50, 40, 30, 20, or 10 nM. In some embodiments, a useful assay is a biochemical assay assessing inhibition of C3 convertase activity by a provided compound. In some embodiments, a purified, isolated or enriched C3 convertase is utilized. In some embodiments, CVF:Bb (e.g., assembled from three components Cobra Venom Factor (CVF), Complement Factor B (FB) and Complement Factor D (FD)) was utilized (certain results are presented as “A” in the Table below). In some embodiments, a compound was first contacted with CVF:Bb, followed by addition of C3 substrates. As those skilled in the art will appreciate, products C3a and/or C3b could be detected through a number of technologies (e.g., antibodies specific for a product) and reduction of levels of one or both of them could be utilized to assess inhibition. In some embodiments, a useful assay is Wieslab© assay (certain results are presented as “B” in the Table below).

In some embodiments, classical and alternative pathway activity are assessed using Wieslab© technologies. In some embodiments, Wieslab© assay were performed with a commercially available kit, using a protocol adapted for measurement of complement inhibitory activity of compounds. In some embodiments, human serum was utilized as a positive control. In some embodiments, human serum serves as a source of complement proteins, and inhibition of complement activity of a serum by various compounds were assessed. In some embodiments, a kit is COMPL AP330, Complement Alternative Pathway ELISA, Eagle Biosciences. In some embodiments, a kit is COMPL CP310, Complement Classical Pathway ELISA, Eagle Biosciences. In some embodiments, kits are available from VWR (102994-104, Complement Alternative pathway ELISA, Eagle Biosciences, Inc.; 102994-106, Complement Classical pathway ELISA, Eagle Biosciences, Inc.).

In some embodiments, a useful procedure described below is utilized.
1. Make fresh test compound stock at 1 mM.
2. Test compound serial dilution: dilute the stock to working stock concentrations. Add DMSO to dilution buffer using same dilution factor for the working stock so the composition of all the serial diluted stock is the same. In some embodiments, stock was serial diluted from the working stock 1:4 (for classical pathway) or 1:2 (for alternative pathway) using new diluted dilution buffers.
3. Dilute positive controls using the dilution buffer provided with the assay kit to 2× of the final conc., for classical pathway, 50.5×. Positive control is human serum, which contains complement proteins.
4. Add 125 μL of test compound solution and 125 μL of diluted positive control to a 96 well polypropylene plate, mix gently and cover the plate and incubate at 4° C. for 30 min.
5. Add 100 μL/well to the assay plate and incubate at 37° C. for 1 h. Typically, controls in the assay plate, for example, buffer alone for negative control, positive control alone to verify the complement activity of the reagent.
6. Wash plate and add 100 μL/well conjugate, incubate at room temperature for 30 min.
7. Wash plate and add 100 μL/well substrate, then incubate at room temperature for approximately 30 minutes. Periodically monitor the absorbance at 405 nm (OD405) in the positive control alone well.
8. Once the OD405 is >1, read the whole plate immediately. Or stop the reaction by adding 100 μL of 5 mM EDTA to each well and read within 60 minutes.

Certain results are provided below. A: C3 Convertase Assay IC₅₀ (nM). B: Wieslab AP IC50 (nM) (alternative pathway). NA: IC₅₀ greater than 10 uM. As demonstrated herein, various compounds can inhibit C3 convertase activity.

TABLE E-1
Certain compounds and assay data.

| A | B | Compound |
|---|---|---|
| 5242 | | 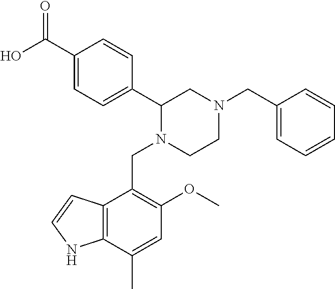 [SMILES: COc1cc(C)c2[nH]ccc2c1CN1CCN(Cc2ccccc2)CC1c1ccc(C(=O)O)cc1] |
| 163 | 1572 | 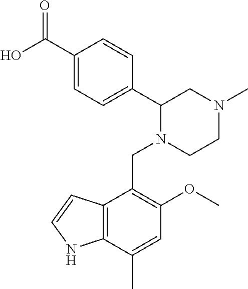 [SMILES: COc1cc(C)c2[nH]ccc2c1CN1CCN(C)CC1c1ccc(C(=O)O)cc1] |
| 9332 | | 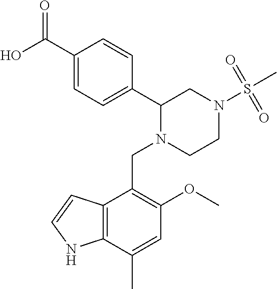 [SMILES: COc1cc(C)c2[nH]ccc2c1CN1CCN(S(C)(=O)=O)CC1c1ccc(C(=O)O)cc1] |
| NA | | 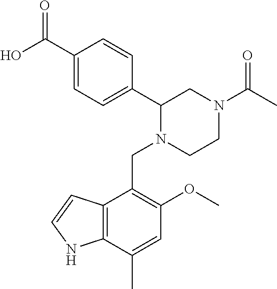 [SMILES: COc1cc(C)c2[nH]ccc2c1CN1CCN(C(C)=O)CC1c1ccc(C(=O)O)cc1] |
| NA | | 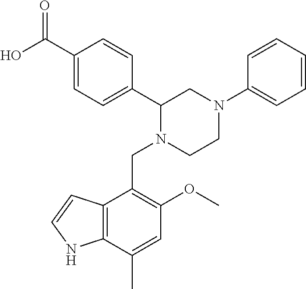 [SMILES: COc1cc(C)c2[nH]ccc2c1CN1CCN(c2ccccc2)CC1c1ccc(C(=O)O)cc1] |
| NA | | 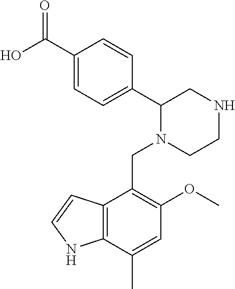 [SMILES: COc1cc(C)c2[nH]ccc2c1CN1CCNCC1c1ccc(C(=O)O)cc1] |
| 652 | | 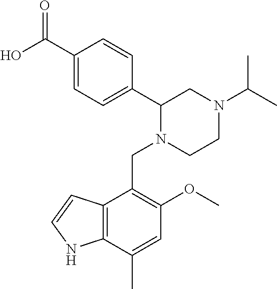 [SMILES: COc1cc(C)c2[nH]ccc2c1CN1CCN(C(C)C)CC1c1ccc(C(=O)O)cc1] |
| 2290 | | 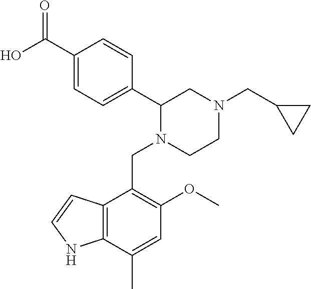 [SMILES: COc1cc(C)c2[nH]ccc2c1CN1CCN(CC2CC2)CC1c1ccc(C(=O)O)cc1] |
| 4667 | | 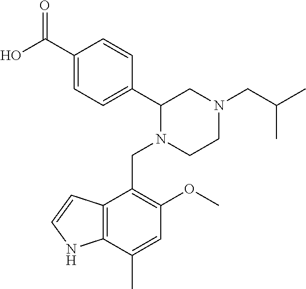 [SMILES: COc1cc(C)c2[nH]ccc2c1CN1CCN(CC(C)C)CC1c1ccc(C(=O)O)cc1] |
| 2584 | | 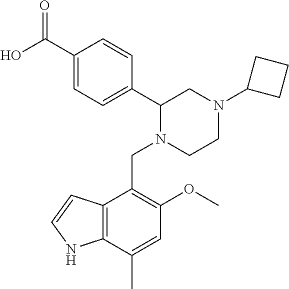 [SMILES: COc1cc(C)c2[nH]ccc2c1CN1CCN(C2CCC2)CC1c1ccc(C(=O)O)cc1] |
| 644 | | 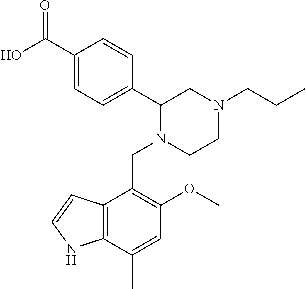 [SMILES: CCCN1CCN(Cc2c(OC)cc(C)c3[nH]ccc23)C(c2ccc(C(=O)O)cc2)C1] |
| NA | | 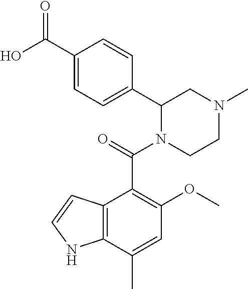 [SMILES: COc1cc(C)c2[nH]ccc2c1C(=O)N1CCN(C)CC1c1ccc(C(=O)O)cc1] |
| 3884 | | 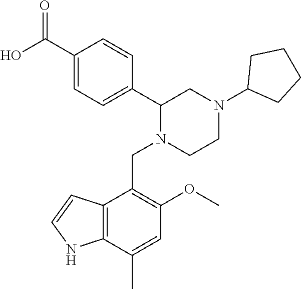 [SMILES: COc1cc(C)c2[nH]ccc2c1CN1CCN(C2CCCC2)CC1c1ccc(C(=O)O)cc1] |
| 405 | | 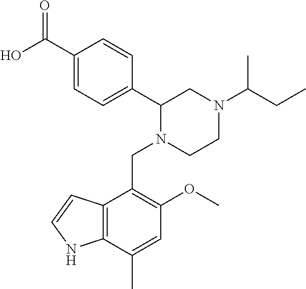 [SMILES: CCC(C)N1CCN(Cc2c(OC)cc(C)c3[nH]ccc23)C(c2ccc(C(=O)O)cc2)C1] |
| 980 | | 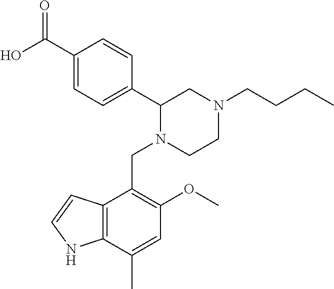 [SMILES: CCCCN1CCN(Cc2c(OC)cc(C)c3[nH]ccc23)C(c2ccc(C(=O)O)cc2)C1] |
| 6585 | | 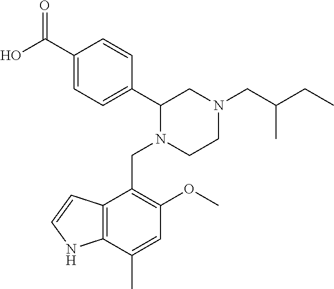 [SMILES: CCC(C)CN1CCN(Cc2c(OC)cc(C)c3[nH]ccc23)C(c2ccc(C(=O)O)cc2)C1] |
| 3195 | | 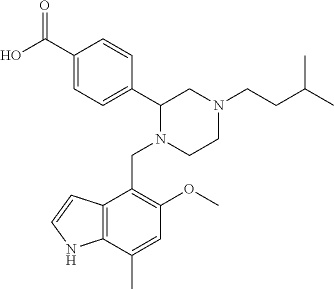 [SMILES: COc1cc(C)c2[nH]ccc2c1CN1CCN(CCC(C)C)CC1c1ccc(C(=O)O)cc1] |
| 3083 | | 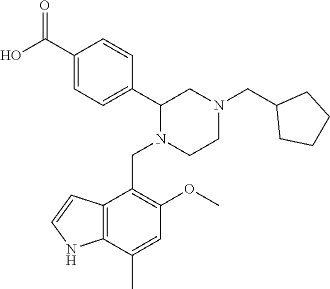 [SMILES: COc1cc(C)c2[nH]ccc2c1CN1CCN(CC2CCCC2)CC1c1ccc(C(=O)O)cc1] |
| NA | | 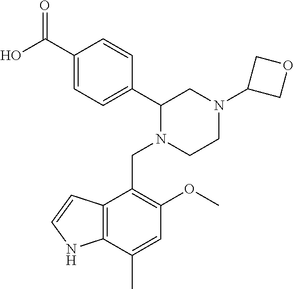 [SMILES: COc1cc(C)c2[nH]ccc2c1CN1CCN(C2COC2)CC1c1ccc(C(=O)O)cc1] |
| 119 | 153 | 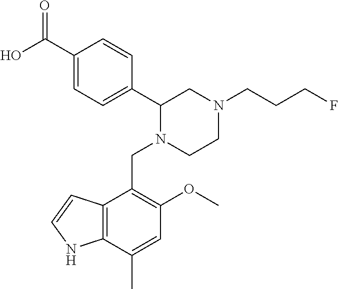 [SMILES: COc1cc(C)c2[nH]ccc2c1CN1CCN(CCCF)CC1c1ccc(C(=O)O)cc1] |
| 219 | 205 | 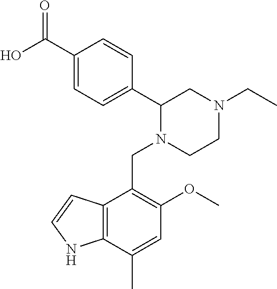 [SMILES: CCN1CCN(Cc2c(OC)cc(C)c3[nH]ccc23)C(c2ccc(C(=O)O)cc2)C1] |
| NA | | 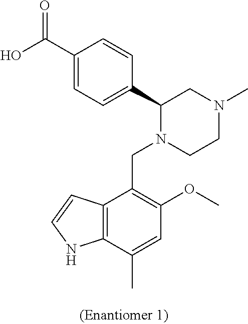 [SMILES: COc1cc(C)c2[nH]ccc2c1CN1CCN(C)C[C@@H]1c1ccc(C(=O)O)cc1] |
| 45 | 64 | 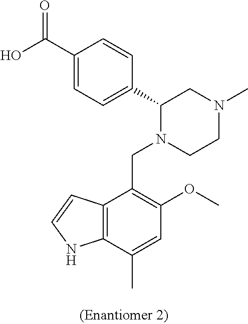 [SMILES: COc1cc(C)c2[nH]ccc2c1CN1CCN(C)C[C@H]1c1ccc(C(=O)O)cc1] |
| 141 | 29 | 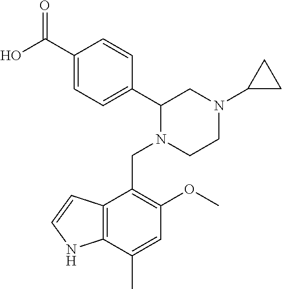 [SMILES: COc1cc(C)c2[nH]ccc2c1CN1CCN(C2CC2)CC1c1ccc(C(=O)O)cc1] |
| 479 | | 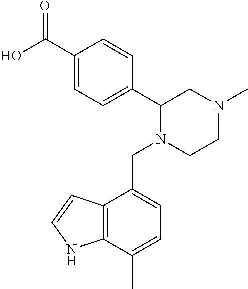 [SMILES: Cc1ccc(CN2CCN(C)CC2c2ccc(C(=O)O)cc2)c2cc[nH]c12] |
| NA | | 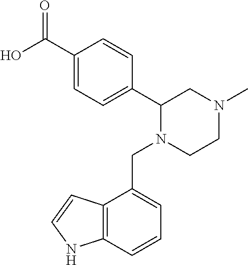 [SMILES: CN1CCN(Cc2cccc3[nH]ccc23)C(c2ccc(C(=O)O)cc2)C1] |
| 250 | 1493 | 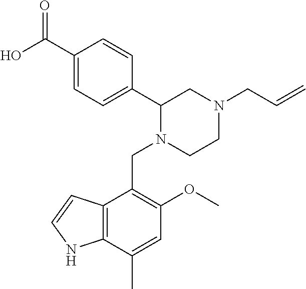 [SMILES: C=CCN1CCN(Cc2c(OC)cc(C)c3[nH]ccc23)C(c2ccc(C(=O)O)cc2)C1] |
| 1071 | | 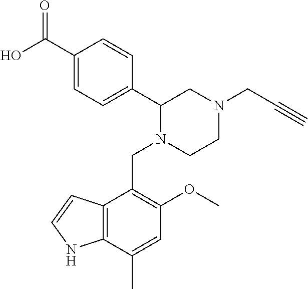 [SMILES: C#CCN1CCN(Cc2c(OC)cc(C)c3[nH]ccc23)C(c2ccc(C(=O)O)cc2)C1] |
| 119 | | 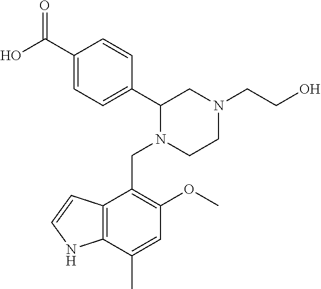 [SMILES: COc1cc(C)c2[nH]ccc2c1CN1CCN(CCO)CC1c1ccc(C(=O)O)cc1] |
| 1348 | | 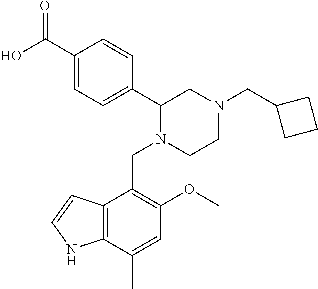 [SMILES: COc1cc(C)c2[nH]ccc2c1CN1CCN(CC2CCC2)CC1c1ccc(C(=O)O)cc1] |
| 65 | 107 | 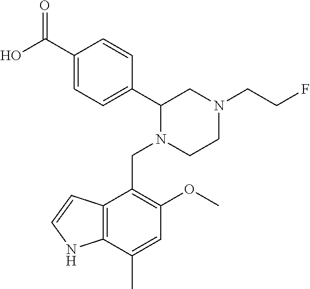 [SMILES: COc1cc(C)c2[nH]ccc2c1CN1CCN(CCF)CC1c1ccc(C(=O)O)cc1] |
| 76 | 36 | 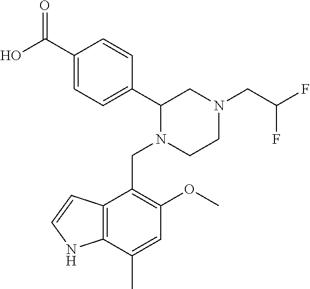 [SMILES: COc1cc(C)c2[nH]ccc2c1CN1CCN(CC(F)F)CC1c1ccc(C(=O)O)cc1] |
| 221 | | 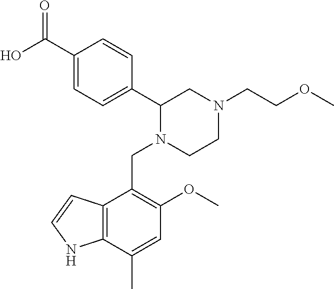 [SMILES: COCCN1CCN(Cc2c(OC)cc(C)c3[nH]ccc23)C(c2ccc(C(=O)O)cc2)C1] |
| 4376 | | 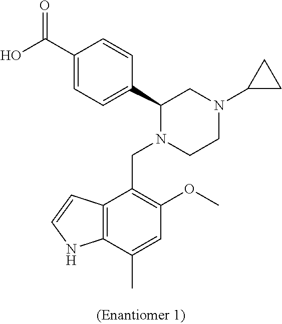 [SMILES: COc1cc(C)c2[nH]ccc2c1CN1CCN(C2CC2)C[C@@H]1c1ccc(C(=O)O)cc1] |
| 96 | | 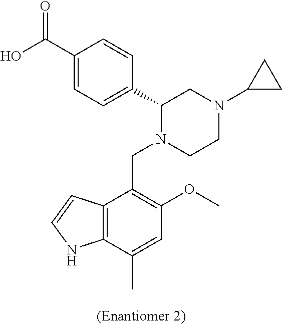 [SMILES: COc1cc(C)c2[nH]ccc2c1CN1CCN(C2CC2)C[C@H]1c1ccc(C(=O)O)cc1] |
| 42 | | 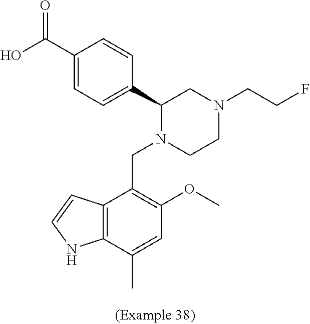 [SMILES: COc1cc(C)c2[nH]ccc2c1CN1CCN(CCF)C[C@@H]1c1ccc(C(=O)O)cc1] |
| 28 | | 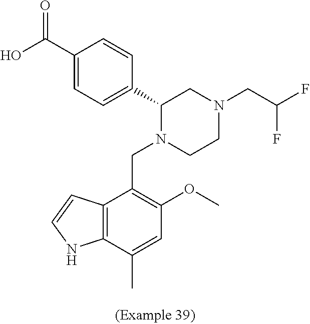 [SMILES: COc1cc(C)c2[nH]ccc2c1CN1CCN(CC(F)F)C[C@H]1c1ccc(C(=O)O)cc1] |
| 96 | 34 | 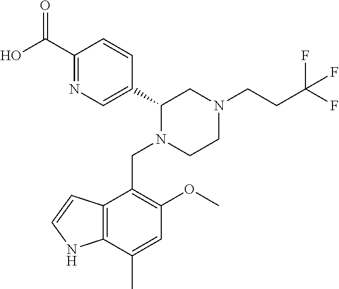 [SMILES: COc1cc(C)c2[nH]ccc2c1CN1CCN(CCC(F)(F)F)C[C@H]1c1ccc(C(=O)O)nc1] |
| 6440 | NA | 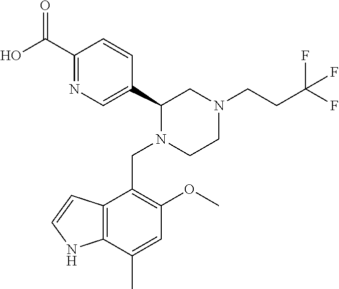 [SMILES: COc1cc(C)c2[nH]ccc2c1CN1CCN(CCC(F)(F)F)C[C@@H]1c1ccc(C(=O)O)nc1] |
| NA | 8865 | 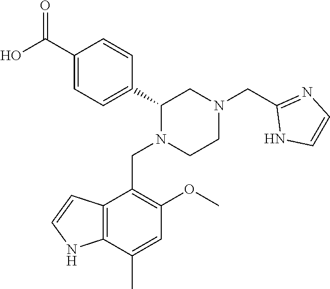 [SMILES: COc1cc(C)c2[nH]ccc2c1CN1CCN(Cc2ncc[nH]2)C[C@H]1c1ccc(C(=O)O)cc1] |
| | NA | 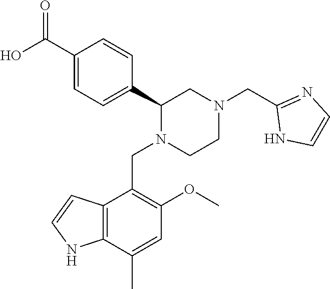 [SMILES: COc1cc(C)c2[nH]ccc2c1CN1CCN(Cc2ncc[nH]2)C[C@@H]1c1ccc(C(=O)O)cc1] |
| 9115 | | 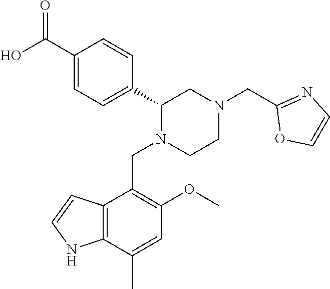 [SMILES: COc1cc(C)c2[nH]ccc2c1CN1CCN(Cc2ncco2)C[C@H]1c1ccc(C(=O)O)cc1] |
| NA | | 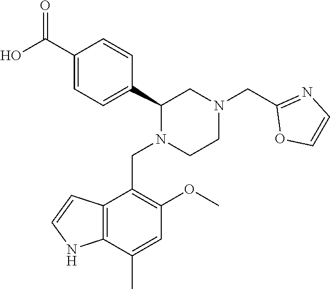 [SMILES: COc1cc(C)c2[nH]ccc2c1CN1CCN(Cc2ncco2)C[C@@H]1c1ccc(C(=O)O)cc1] |
| | 110 | 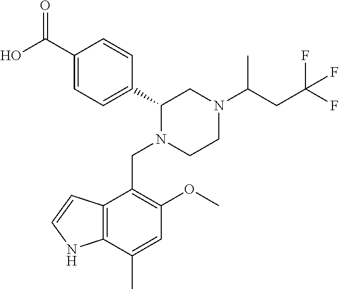 [SMILES: COc1cc(C)c2[nH]ccc2c1CN1CCN(C(C)CC(F)(F)F)C[C@H]1c1ccc(C(=O)O)cc1] |
| 1490 | | 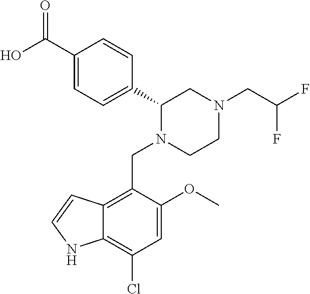 [SMILES: COc1cc(Cl)c2[nH]ccc2c1CN1CCN(CC(F)F)C[C@H]1c1ccc(C(=O)O)cc1] |
| 348 | | 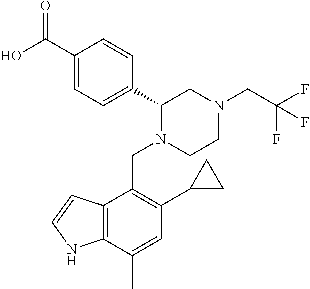 [SMILES: Cc1cc(C2CC2)c(CN2CCN(CC(F)(F)F)C[C@H]2c2ccc(C(=O)O)cc2)c2cc[nH]c12] |
| NA | | 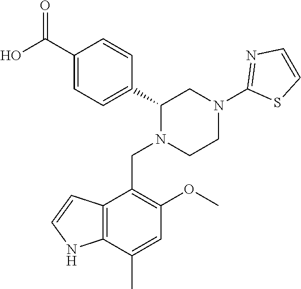 [SMILES: COc1cc(C)c2[nH]ccc2c1CN1CCN(c2nccs2)C[C@H]1c1ccc(C(=O)O)cc1] |
| NA | | 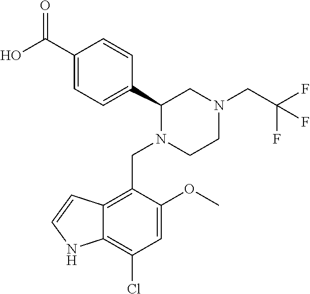 [SMILES: COc1cc(Cl)c2[nH]ccc2c1CN1CCN(CC(F)(F)F)C[C@@H]1c1ccc(C(=O)O)cc1] |
| 2349 | | 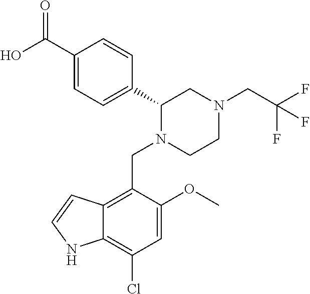 [SMILES: COc1cc(Cl)c2[nH]ccc2c1CN1CCN(CC(F)(F)F)C[C@H]1c1ccc(C(=O)O)cc1] |
| 2702 | | 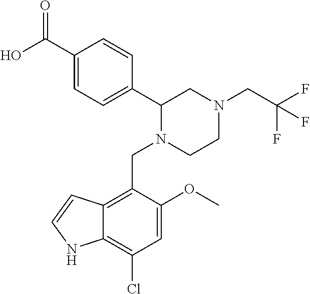 [SMILES: COc1cc(Cl)c2[nH]ccc2c1CN1CCN(CC(F)(F)F)CC1c1ccc(C(=O)O)cc1] |
| 7395 | | 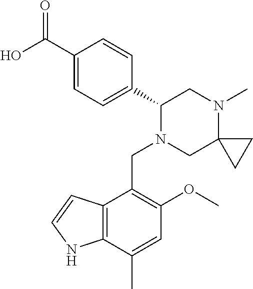 [SMILES: COc1cc(C)c2[nH]ccc2c1CN1CC2(CC2)N(C)C[C@H]1c1ccc(C(=O)O)cc1] |
| 56 | 65 | 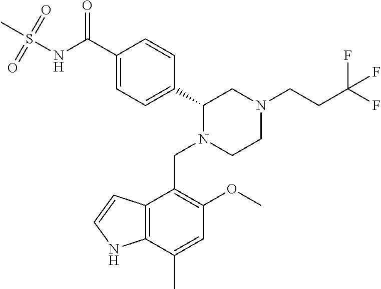 [SMILES: COc1cc(C)c2[nH]ccc2c1CN1CCN(CCC(F)(F)F)C[C@H]1c1ccc(C(=O)NS(C)(=O)=O)cc1] |
| 212 | 382 | 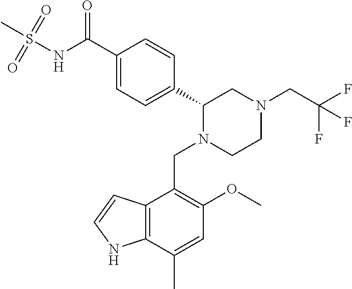 [SMILES: COc1cc(C)c2[nH]ccc2c1CN1CCN(CC(F)(F)F)C[C@H]1c1ccc(C(=O)NS(C)(=O)=O)cc1] |
| 2161 | | 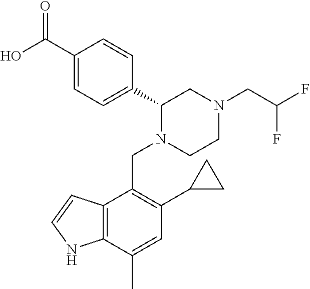 [SMILES: Cc1cc(C2CC2)c(CN2CCN(CC(F)F)C[C@H]2c2ccc(C(=O)O)cc2)c2cc[nH]c12] |
| 3147 | | 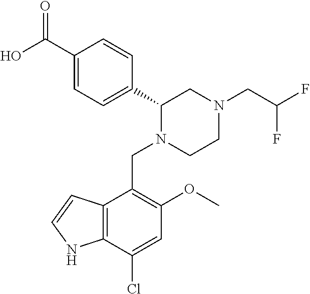 [SMILES: COc1cc(Cl)c2[nH]ccc2c1CN1CCN(CC(F)F)C[C@H]1c1ccc(C(=O)O)cc1] |
| 520 | | 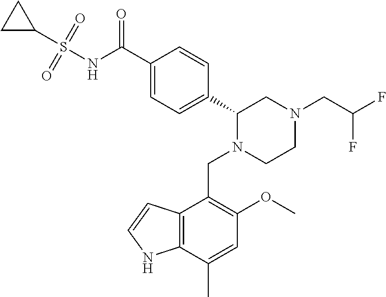 [SMILES: COc1cc(C)c2[nH]ccc2c1CN1CCN(CC(F)F)C[C@H]1c1ccc(C(=O)NS(=O)(=O)C2CC2)cc1] |
| 163 | | 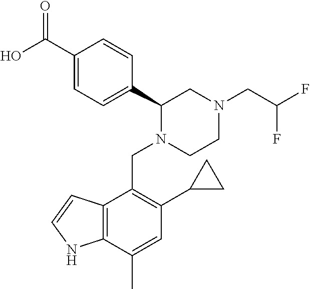 [SMILES: Cc1cc(C2CC2)c(CN2CCN(CC(F)F)C[C@@H]2c2ccc(C(=O)O)cc2)c2cc[nH]c12] |
| 80 | | 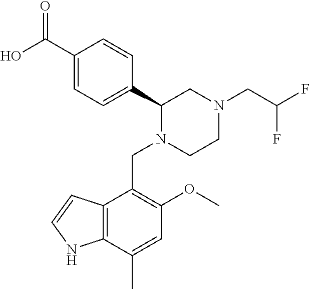 [SMILES: COc1cc(C)c2[nH]ccc2c1CN1CCN(CC(F)F)C[C@@H]1c1ccc(C(=O)O)cc1] |
| 136 | 396 | 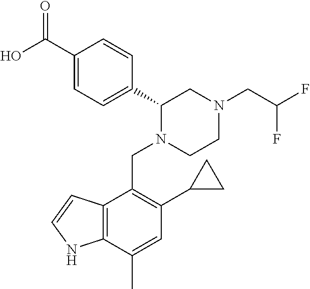 [SMILES: Cc1cc(C2CC2)c(CN2CCN(CC(F)F)C[C@H]2c2ccc(C(=O)O)cc2)c2cc[nH]c12] |
| NA | | 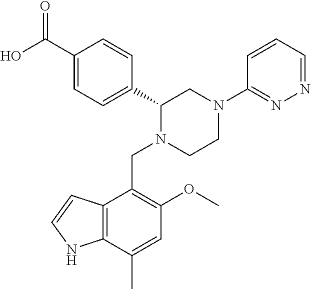 [SMILES: COc1cc(C)c2[nH]ccc2c1CN1CCN(c2cccnn2)C[C@H]1c1ccc(C(=O)O)cc1] |
| 272 | 211 | 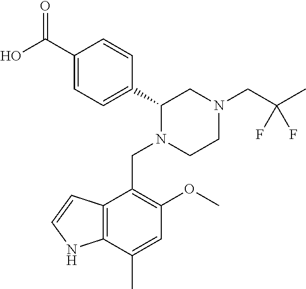 [SMILES: COc1cc(C)c2[nH]ccc2c1CN1CCN(CC(C)(F)F)C[C@H]1c1ccc(C(=O)O)cc1] |
| 27 | 27 | 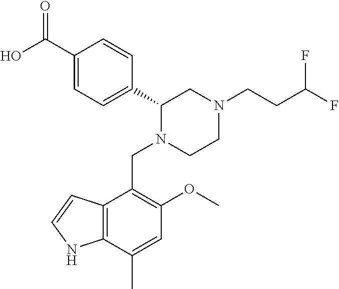 [SMILES: COc1cc(C)c2[nH]ccc2c1CN1CCN(CCC(F)F)C[C@H]1c1ccc(C(=O)O)cc1] |
| 6402 | 1000 | 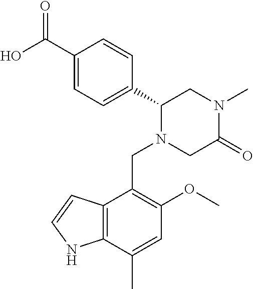 [SMILES: COc1cc(C)c2[nH]ccc2c1CN1CC(=O)N(C)C[C@H]1c1ccc(C(=O)O)cc1] |
| 360 | 1000 | 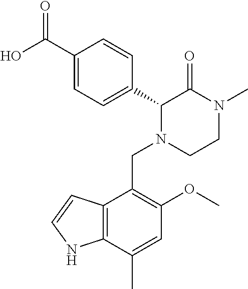 [SMILES: COc1cc(C)c2[nH]ccc2c1CN1CCN(C)C(=O)[C@H]1c1ccc(C(=O)O)cc1] |
| 228 | 230 | 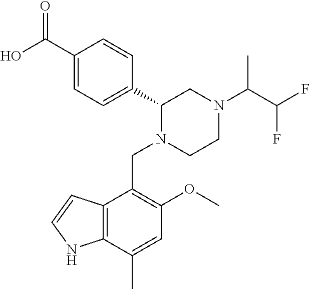 [SMILES: COc1cc(C)c2[nH]ccc2c1CN1CCN(C(C)C(F)F)C[C@H]1c1ccc(C(=O)O)cc1] |
| 99 | | 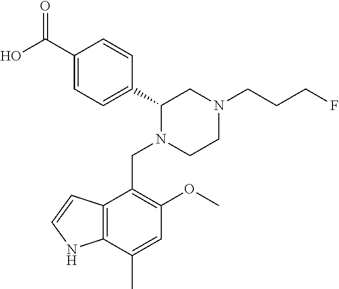 [SMILES: COc1cc(C)c2[nH]ccc2c1CN1CCN(CCCF)C[C@H]1c1ccc(C(=O)O)cc1] |
| 350 | 128 | 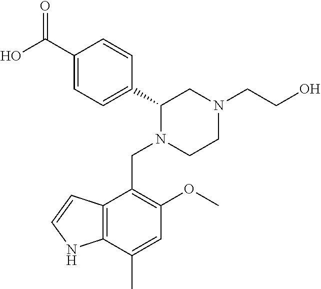 [SMILES: COc1cc(C)c2[nH]ccc2c1CN1CCN(CCO)C[C@H]1c1ccc(C(=O)O)cc1] |
| 65 | 9 | 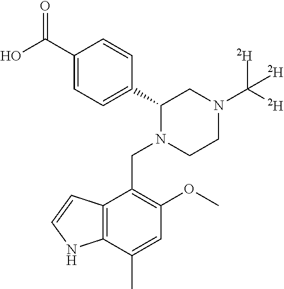 [SMILES: [2H]C([2H])([2H])N1CCN(Cc2c(OC)cc(C)c3[nH]ccc23)[C@H](c2ccc(C(=O)O)cc2)C1] |
| NA | | 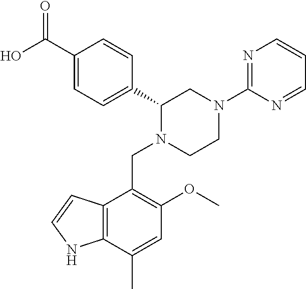 [SMILES: COc1cc(C)c2[nH]ccc2c1CN1CCN(c2ncccn2)C[C@H]1c1ccc(C(=O)O)cc1] |
| 29 | 13 | 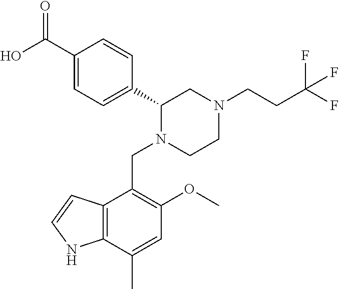 [SMILES: COc1cc(C)c2[nH]ccc2c1CN1CCN(CCC(F)(F)F)C[C@H]1c1ccc(C(=O)O)cc1] |
| 243 | 8 | 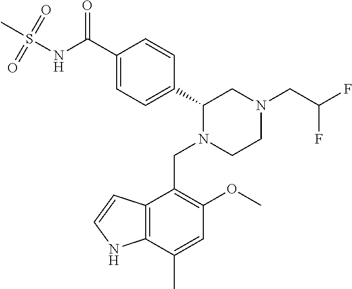 [SMILES: COc1cc(C)c2[nH]ccc2c1CN1CCN(CC(F)F)C[C@H]1c1ccc(C(=O)NS(C)(=O)=O)cc1] |
| 176 | 174 | 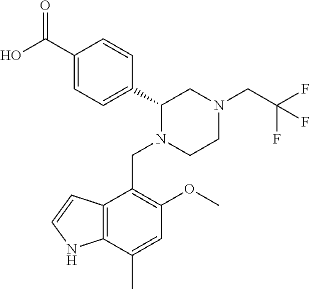 [SMILES: COc1cc(C)c2[nH]ccc2c1CN1CCN(CC(F)(F)F)C[C@H]1c1ccc(C(=O)O)cc1] |

### EQUIVALENTS

Those skilled in the art will recognize, or be able to ascertain using no more than routine experimentation, many equivalents to specific embodiments of the present disclosure.

1. A compound having the structure of formula I: 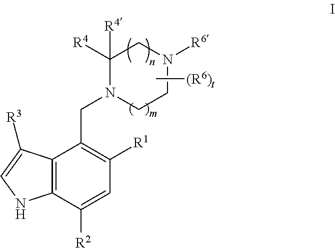
or a pharmaceutically acceptable salt thereof, wherein:
m is 0, 1, or 2;
n is 1, or 2;
R¹ is hydrogen, halogen, hydroxy, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷, phenyl or 5- or 6-membered heteroaryl having 1, 2 or 3 ring heteroatoms independently selected from nitrogen, oxygen and sulfur, wherein the phenyl or heteroaryl is optionally substituted with 0, 1, or 2 C₁-C₄ alkyl groups, and wherein alkyl and haloalkyl are optionally substituted with 0 or 1 hydroxy;
R⁴ is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl;
R^{4′} is hydrogen, C₁-C₄ alkyl, or hydroxy C₁-C₄ alkyl;
R⁵ is —C(O)R⁸, —CH₂C(O)R¹, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁—C₄ alkyl, —B(R′)₂ or 5-membered heteroaryl having 1-4 ring nitrogen atoms and 0 or 1 ring oxygen or sulfur atom;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
R⁷ is hydroxy, C₁-C₄ alkoxy, amino, or mono- or di-C₁-C₄ alkylamino;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.

2. A compound having the structure of formula M-I: 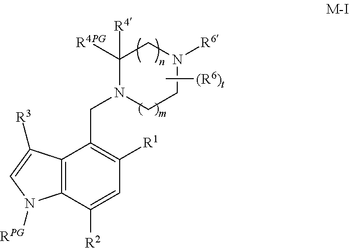
or a salt thereof, wherein:
R^{PG} is an amino protecting group,
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 0, 1, or 2;
n is 1, or 2;
R¹ is hydrogen, halogen, hydroxy, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷, phenyl or 5- or 6-membered heteroaryl having 1, 2 or 3 ring heteroatoms independently selected from nitrogen, oxygen and sulfur, wherein the phenyl or heteroaryl is optionally substituted with 0, 1, or 2 C₁-C₄ alkyl groups, and wherein alkyl and haloalkyl are optionally substituted with 0 or 1 hydroxy;
R^{4′} is hydrogen, C₁-C₄ alkyl, or hydroxy C₁-C₄ alkyl;
R⁵ is —C(O)R⁸, —CH₂C(O)R¹, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
R⁷ is hydroxy, C₁-C₄ alkoxy, amino, or mono- or di-C₁-C₄ alkylamino;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-II: 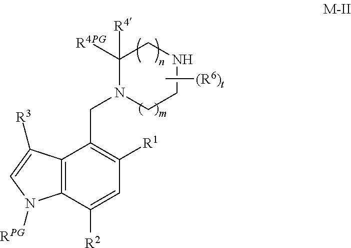
or a salt thereof, wherein:
R^{PG} is an amino protecting group,
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 0, 1, or 2;
n is 1, or 2;
R¹ is hydrogen, halogen, hydroxy, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷, phenyl or 5- or 6-membered heteroaryl having 1, 2 or 3 ring heteroatoms independently selected from nitrogen, oxygen and sulfur, wherein the phenyl or heteroaryl is optionally substituted with 0, 1, or 2 C₁-C₄ alkyl groups, and wherein alkyl and haloalkyl are optionally substituted with 0 or 1 hydroxy;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R⁷ is hydroxy, C₁-C₄ alkoxy, amino, or mono- or di-C₁-C₄ alkylamino;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-III: 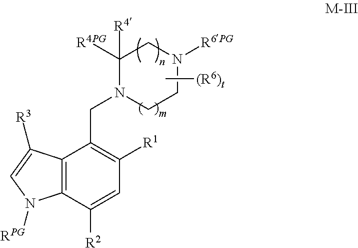
or a salt thereof, wherein:
R^{PG} is an amino protecting group,
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 0, 1, or 2;
n is 1, or 2;
R¹ is hydrogen, halogen, hydroxy, C₁-C₆ alkyl, C₂-C₆ alkenyl, C₃-C₆ cycloalkyl, C₁-C₆ alkoxy, halo C₁-C₆ alkyl, hydroxy C₁-C₆ alkyl, amino C₁-C₆ alkyl, C₁-C₆ alkoxy C₁-C₆ alkyl, C₁-C₆ alkoxy C₁-C₆ alkoxy, C₃-C₆ cycloalkyl C₁-C₆ alkoxy, halo C₁-C₆ alkoxy, —S(O)ₚC₁-C₆ alkyl, —CH₂NHC(O)C₁-C₄ alkyl or —OCH₂C(O)R⁷;
p is 0, 1, or 2;
R² is hydrogen, C₁-C₆ alkyl, C₁-C₆ alkoxy, hydroxy C₁-C₆ alkyl, or halogen;
R³ is hydrogen, halogen, cyano, C₁-C₄ alkyl, halo C₁-C₄ alkyl, —CH₂C(O)R⁷, phenyl or 5- or 6-membered heteroaryl having 1, 2 or 3 ring heteroatoms independently selected from nitrogen, oxygen and sulfur, wherein the phenyl or heteroaryl is optionally substituted with 0, 1, or 2 C₁-C₄ alkyl groups, and wherein alkyl and haloalkyl are optionally substituted with 0 or 1 hydroxy;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
R⁷ is hydroxy, C₁-C₄ alkoxy, amino, or mono- or di-C₁-C₄ alkylamino;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.

3. The compound of claim 1 or 2, wherein R¹ is C₁-C₆ alkoxy.

4. The compound of claim 3, wherein R¹ is methoxy.

5. The compound of claim 3, wherein R² is C₁-C₆ alkyl.

6. The compound of claim 5, wherein R² is methyl.

7. The compound of claim 5, wherein R³ is hydrogen.

8. A compound having the structure of formula M-IV: 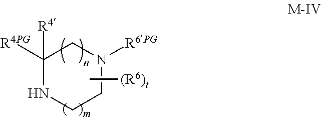
or a salt thereof, wherein:
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 0, 1, or 2;
n is 1, or 2;
p is 0, 1, or 2;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-VI: 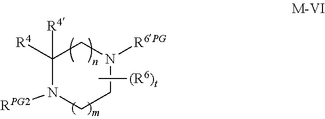
or a salt thereof, wherein:
m is 0, 1, or 2;
n is 1, or 2;
R^{PG2} is an amino protecting group;
R⁴ is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl;
R⁵ is —C(O)R⁸, —CH₂C(O)R¹, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
p is 0, 1, or 2;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-VII: 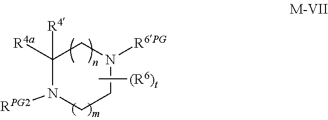
or a salt thereof, wherein:
m is 0, 1, or 2;
n is 1, or 2;
R^{PG2} is an amino protecting group;
R^{4a} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5a} and further substituted with 0 or 1 substituent selected from halogen, C₁-C₄ alkyl, C₁-C₄ alkoxy, hydroxy C₁-C₄ alkyl, hydroxy, and cyanomethyl;
R^{5a} is —CHO, —CH₂CHO, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
p is 0, 1, or 2;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-VIII: 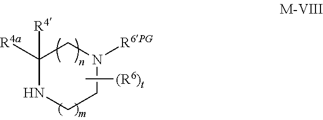
or a salt thereof, wherein:
m is 0, 1, or 2;
n is 1, or 2;
R^{4a} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5a} and further substituted with 0 or 1 substituent selected from halogen, C₁-C₄ alkyl, C₁-C₄ alkoxy, hydroxy C₁-C₄ alkyl, hydroxy, and cyanomethyl;
R^{5a} is —CHO, —CH₂CHO, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
p is 0, 1, or 2;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C1-3 acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-IX: 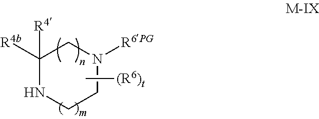
or a salt thereof, wherein:
m is 0, 1, or 2;
n is 1, or 2;
R^{4b} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5b} and further substituted with 0 or 1 substituent selected from halogen, C₁-C₄ alkyl, C₁-C₄ alkoxy, hydroxy C₁-C₄ alkyl, hydroxy, and cyanomethyl;
R^{5b} is —CH₂OH, —CH₂ CH₂OH, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R⁸)₂ or 5-membered heteroaryl having 1-4 ring nitrogen atoms and 0 or 1 ring oxygen or sulfur atom;
R^{4′} is hydrogen, C₁-C₄ alkyl, or hydroxy C₁-C₄ alkyl;
p is 0, 1, or 2;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-X: 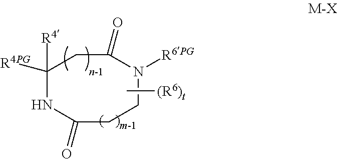
or a salt thereof, wherein:
m is 1, or 2;
n is 1, or 2;
R^{4′} is hydrogen, C₁-C₄ alkyl, or hydroxy C₁-C₄ alkyl;
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-XI: 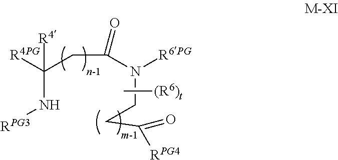
or a salt thereof, wherein:
m is 1, or 2;
n is 1, or 2;
R^{PG3} is —H or an amino protecting group;
R^{PG4} is —OH or —C(O)R^{PG4} is a protected carboxyl group;
R^{4′} is hydrogen, C₁-C₄ alkyl, or hydroxy C₁-C₄ alkyl;
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-XII: 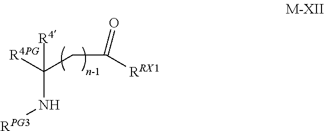
or a salt thereof, wherein:
n is 1, or 2;
R^{PG3} is —H or an amino protecting group;
R^{4′} is hydrogen, C₁-C₄ alkyl, or hydroxy C₁-C₄ alkyl;
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2;
R^{RX1} is —OH or —C(O)R^{RX1} is an activated carboxyl group;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl; and
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; or
a compound having the structure of formula M-XIII: 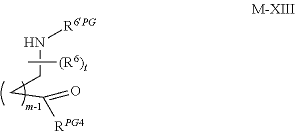
or a salt thereof, wherein:
m is 1, or 2;
R^{PG4} is —OH or —C(O)R^{PG4} is a protected carboxyl group;
R^{6′PG} is R^{6′} or an amino protecting group;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-XIV: 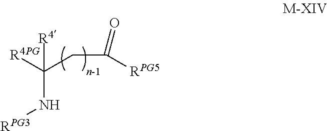
or a salt thereof, wherein:
n is 1, or 2;
R^{PG3} is —H or an amino protecting group;
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2;
—C(O)R^{PG5} is protected carboxyl;
R^{4′} is hydrogen, C₁-C₄ alkyl, or hydroxy C₁-C₄ alkyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl; and
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups;
a compound having the structure of formula M-IV′: 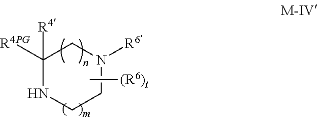
or a salt thereof, wherein:
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
p is 0, 1, or 2;
m is 0, 1, or 2;
n is 1, or 2;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C1-3 acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C1-3 acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur; or
a compound having the structure of formula M-XV: 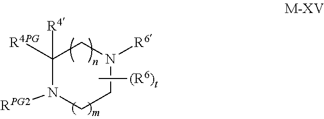
or a salt thereof, wherein:
R^{PG2} is an amino protecting group;
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 0, 1, or 2;
n is 1, or 2;
p is 0, 1, or 2;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur;
a compound having the structure of formula M-XVI: 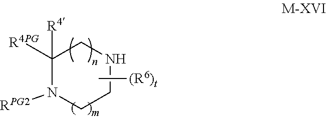
or a salt thereof, wherein:
R^{PG2} is an amino protecting group;
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R^{5PG} and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 0, 1, or 2;
n is 1, or 2;
p is 0, 1, or 2;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl; and
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; or
a compound having the structure of formula M-XVII: 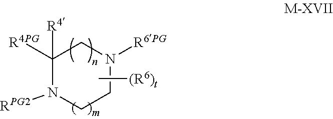
or a salt thereof, wherein:
R^{PG2} is an amino protecting group;
R^{4PG} is phenyl, naphthyl, 1,2,3,4-tetrahydronaphthalenyl or 5- or 6-membered heteroaryl having 1, 2 or 3 ring heteroatoms independently selected from nitrogen, oxygen and sulfur, and where the phenyl, naphthyl, 1,2,3,4-tetrahydronaphthalenyl, or heteroaryl is optionally substituted with R⁵PG and further substituted with 0 or 1 substituent selected from halogen, C₁-C₄ alkyl, C₁-C₄ alkoxy, hydroxy C₁-C₄ alkyl, hydroxy, and cyanomethyl;
R^{5PG} is —C(O)R^{8PG}, —CH₂C(O)R^{8PG}, R⁹, —C(O)NHSO₂C₁-C₄alkyl, —SO₂NHC(O)C₁-C₄alkyl, —SO₂N(H)ₚ(C₁-C₄alkyl)₂₋ₚ, —SO(NH)C₁-C₄alkyl, —SO₂C₁-C₄alkyl, cyano, halogen, hydroxy C₁-C₄ alkyl, —B(R^{8PG})₂ or 5-membered heteroaryl having 1-4 ring nitrogen atoms and 0 or 1 ring oxygen or sulfur atom;
each R^{8PG} is independently R⁸, or —C(O)R^{8PG} is a protected carboxylic acid group;
m is 0, 1, or 2;
n is 1, or 2;
p is 0, 1, or 2;
R^{4′} is hydrogen, C₁-C₄ alkyl, or hydroxy C₁-C₄ alkyl;
each R⁶ is independently hydrogen, halogen, hydroxy, amino, mono- and di-C₁-C₆ alkylamino, C₁-C₆ alkyl, halo C₁-C₆ alkyl, hydroxy C₁-C₆ alkyl, cyano C₁-C₆ alkyl or C₁-C₆ alkoxy, or:
two R⁶ on a carbon atom are taken together to form ═O, or two R⁶ on a carbon atom are taken together with the carbon atom to form a 3-membered monocyclic saturated ring having 0-1 ring atoms independently selected from nitrogen, sulfur and oxygen; or
two R⁶ are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
t is 0, 1, 2, 3 or 4;
R^{6′PG} is R^{6′} or an amino protecting group;
R^{6′} is hydrogen, C₁-C₆ aliphatic, R¹⁰, —CH₂—C₃-C₆ cycloaliphatic, —CH₂—R¹⁰, —CH₂-(hydroxy C₁-C₄ alkyl), phenyl, —C(O)—C₁-C₆ aliphatic, —SO₂—C₁-C₆ aliphatic, —CH₂-phenyl, —CH₂-(amino C₁-C₄ alkyl), or —CH₂-(mono- and di-C₁-C₄ alkylamino C₁-C₄ alkyl) wherein each of the C₁-C₆ aliphatic and phenyl is optionally substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl; or:
R⁶ and R^{6′} are taken together with their intervening atoms to form a 3-8 membered monocyclic saturated or partially unsaturated ring having 0-3 ring atoms independently selected from nitrogen, sulfur and oxygen, wherein the ring is optionally substituted with one or more substituents independently selected from halogen, C₁-C₄ alkyl, halo C₁-C₄ alkyl, C₁-C₄ alkoxy, hydroxyl, and carbonyl;
each R⁸ is independently hydroxy, C₁-C₄ alkoxy, amino or a 5-7 membered saturated heterocyclyl having 1, 2, or 3 ring heteroatoms independently selected from nitrogen, oxygen and sulfur; or mono- and di-C₁-C₄ alkylamino which is optionally substituted with one or more substituents independently selected from halogen, hydroxy and C₁-C₄ alkyl;
R⁹ is a 5-membered heteroaryl having 1 to 4 ring nitrogen atoms and 0 or 1 ring oxygen or sulfur atom, which heteroaryl is optionally substituted with 0 to 2 C₁-C₄ alkyl groups; and
R¹⁰ is 3-6 membered heterocyclyl or 5-6 membered heteroaryl having 1-3 ring atoms independently selected from nitrogen, oxygen and sulfur, wherein the heterocyclyl or heteroaryl is optionally substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl or heteroaryl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur.

9. The compound of claim 5, wherein R⁴ is phenyl, naphthyl or 5- or 6-membered heteroaryl, wherein the phenyl, naphthyl or 5- or 6-membered heteroaryl is substituted with R⁵ and further substituted with 0 or 1 substituent selected from halogen, C₁-C₄ alkyl, C₁-C₄ alkoxy, hydroxy C₁-C₄ alkyl, hydroxy, and cyanomethyl

10. The compound of claim 9, wherein R⁵ is —COOH.

11. The compound of claim 10, wherein R^{4′} is hydrogen.

12. The compound of claim 11, wherein t is 0.

13. The compound of claim 12, wherein R^{6′} is C₁-C₆ aliphatic substituted with one or more substituents independently selected from halogen, C₁-C₄ alkyl, C₁-C₄ alkoxy, C₃-C₆ cycloalkyl or 3-6 membered heterocyclyl having 1-4 ring atoms independently selected from nitrogen, oxygen and sulfur, wherein each of the alkyl, alkoxy, cycloalkyl and heterocyclyl is optionally and independently substituted with one or more substituents independently selected from halogen, C₁-C₄ alkyl, halo C₁-C₄ alkyl, C₁-C₄ alkoxy, hydroxyl, and carbonyl, and wherein each nitrogen ring atom of the heterocyclyl is optionally and independently substituted with C₁₋₃ acyl, cyano C₁-C₄ alkyl, C₃-C₆ cycloalkyl, or 3-6 membered heterocyclyl having 1-4 ring atoms independently selected from nitrogen, oxygen and sulfur wherein each ring nitrogen is optionally and independently substituted with C₁₋₃ acyl.

14. The compound of claim 12, wherein R^{6′} is C₁-C₆ aliphatic substituted with one or more halogen.

15. The compound of claim 14, wherein R^{6′} is C₁-C₆ alkyl substituted with one or more —F.

16. The compound of claim 15, wherein R^{6′} is C₁-C₆ linear alkyl substituted with one or more —F.

17. The compound of claim 16, wherein R^{6′} is —CH₂CH₂F, —CH₂CH₂CH₂F, —CH₂CHF₂, —CH₂CH₂CHF₂, —CH₂CF₃, —CH(CH₃)—CHF₂, —CH₂—CF₂—CH₃, —CH(CH₃)—CH₂—CF₃, —CH₂CH₂CF₃, or —CH₂CF═CH₂.

18. The compound of claim 12, wherein R^{6′} is C₁-C₆ aliphatic.

19. The compound of claim 18, wherein R^{6′} is methyl.

20. The compound of claim 18, wherein R^{6′} is —CD₃.

21. The compound of claim 18, wherein R^{6′} is cyclopropyl.

22. The compound of any one of the preceding claims, wherein the compound has the structure of 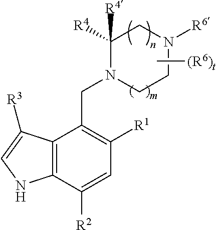
or a pharmaceutically acceptable salt thereof.

23. The compound of any one of the preceding claims, wherein the compound has the structure of: 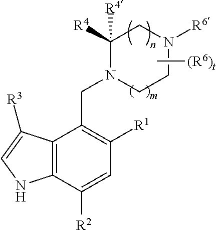
or a pharmaceutically acceptable salt thereof.

24. A compound selected from Table El or a pharmaceutically acceptable salt thereof.

25. The compound of any one of the preceding claims, wherein the compound has an IC₅₀ of about or no more than about 1000, 900, 800, 700, 600, 500, 400, 300, 200, 100, 90, 80, 70, 60, 50, 40, 30, 20, or 10 nM as measured in an assay in the Examples.

26. A compound having the structure of 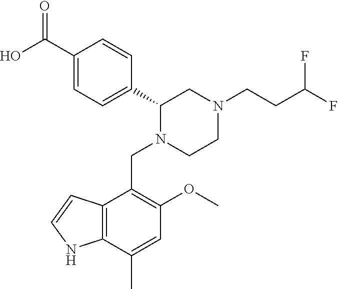 [SMILES: COc1cc(C)c2[nH]ccc2c1CN1CCN(CCC(F)F)C[C@H]1c1ccc(C(=O)O)cc1]
or a pharmaceutically acceptable salt thereof.

27. A compound having the structure of 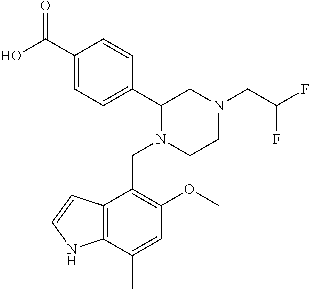 [SMILES: COc1cc(C)c2[nH]ccc2c1CN1CCN(CC(F)F)CC1c1ccc(C(=O)O)cc1]
or a pharmaceutically acceptable salt thereof.

28. A compound having the structure of 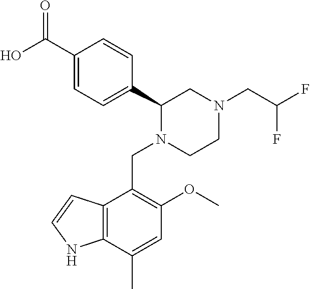 [SMILES: COc1cc(C)c2[nH]ccc2c1CN1CCN(CC(F)F)C[C@@H]1c1ccc(C(=O)O)cc1]
or a pharmaceutically acceptable salt thereof.

29. A compound having the structure of 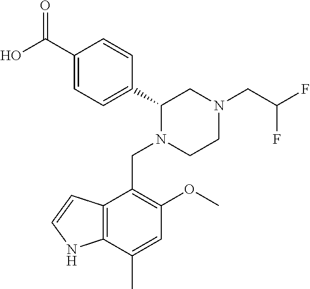 [SMILES: COc1cc(C)c2[nH]ccc2c1CN1CCN(CC(F)F)C[C@H]1c1ccc(C(=O)O)cc1]
or a pharmaceutically acceptable salt thereof.

30. A compound having the structure of 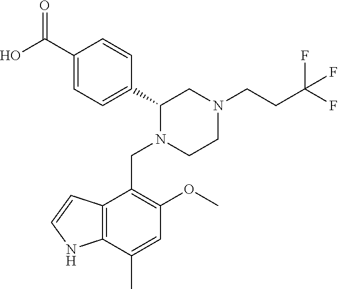 [SMILES: COc1cc(C)c2[nH]ccc2c1CN1CCN(CCC(F)(F)F)C[C@H]1c1ccc(C(=O)O)cc1]
or a pharmaceutically acceptable salt thereof.

31. A compound having the structure of 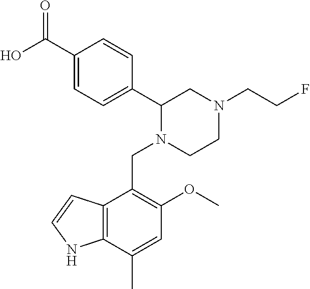 [SMILES: COc1cc(C)c2[nH]ccc2c1CN1CCN(CCF)CC1c1ccc(C(=O)O)cc1]
or a pharmaceutically acceptable salt thereof.

32. A compound having the structure of 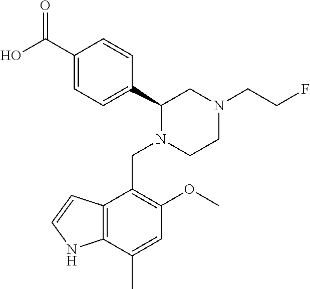 [SMILES: COc1cc(C)c2[nH]ccc2c1CN1CCN(CCF)C[C@@H]1c1ccc(C(=O)O)cc1]
or a pharmaceutically acceptable salt thereof.

33. A compound having the structure of 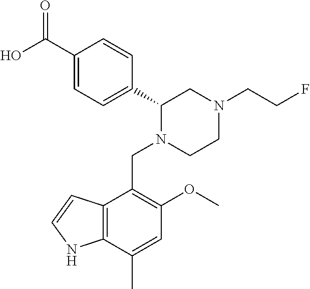 [SMILES: COc1cc(C)c2[nH]ccc2c1CN1CCN(CCF)C[C@H]1c1ccc(C(=O)O)cc1]
or a pharmaceutically acceptable salt thereof.

34. A compound having the structure of 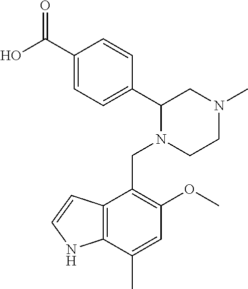 [SMILES: COc1cc(C)c2[nH]ccc2c1CN1CCN(C)CC1c1ccc(C(=O)O)cc1]
or a pharmaceutically acceptable salt thereof.

35. A compound having the structure of 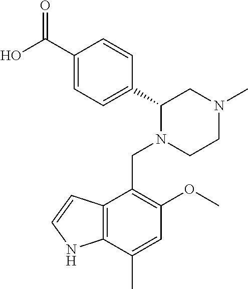 [SMILES: COc1cc(C)c2[nH]ccc2c1CN1CCN(C)C[C@H]1c1ccc(C(=O)O)cc1]
or a pharmaceutically acceptable salt thereof.

36. A compound having the structure of 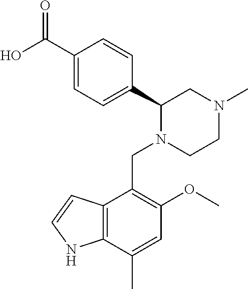 [SMILES: COc1cc(C)c2[nH]ccc2c1CN1CCN(C)C[C@@H]1c1ccc(C(=O)O)cc1]
or a pharmaceutically acceptable salt thereof.

37. A compound having the structure of 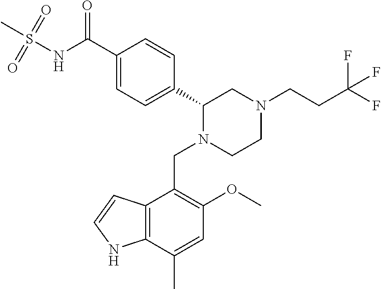 [SMILES: COc1cc(C)c2[nH]ccc2c1CN1CCN(CCC(F)(F)F)C[C@H]1c1ccc(C(=O)NS(C)(=O)=O)cc1]
or a pharmaceutically acceptable salt thereof.

38. A compound having the structure of 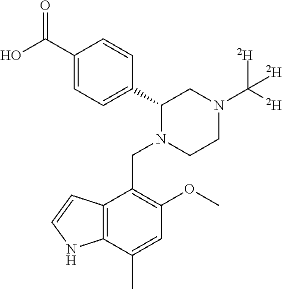 [SMILES: [2H]C([2H])([2H])N1CCN(Cc2c(OC)cc(C)c3[nH]ccc23)[C@H](c2ccc(C(=O)O)cc2)C1]
or a pharmaceutically acceptable salt thereof.

39. A compound having the structure of 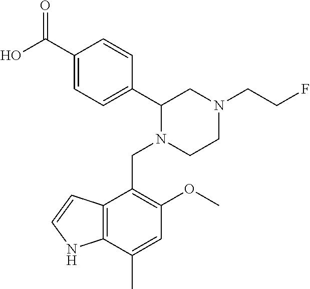 [SMILES: COc1cc(C)c2[nH]ccc2c1CN1CCN(CCF)CC1c1ccc(C(=O)O)cc1]
or a pharmaceutically acceptable salt thereof.

40. A compound having the structure of 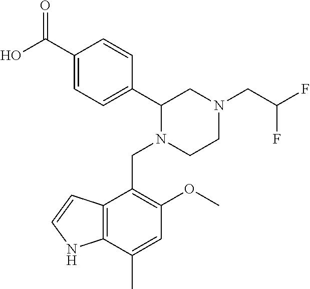 [SMILES: COc1cc(C)c2[nH]ccc2c1CN1CCN(CC(F)F)CC1c1ccc(C(=O)O)cc1]
or a pharmaceutically acceptable salt thereof.

41. A compound having the structure of 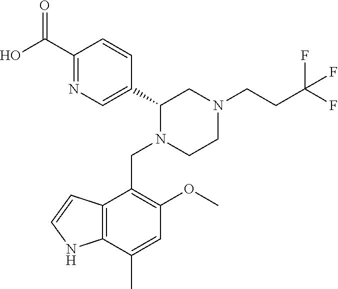 [SMILES: COc1cc(C)c2[nH]ccc2c1CN1CCN(CCC(F)(F)F)C[C@H]1c1ccc(C(=O)O)nc1]
or a pharmaceutically acceptable salt thereof.

42. A compound having the structure of 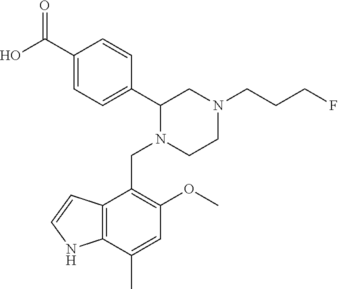 [SMILES: COc1cc(C)c2[nH]ccc2c1CN1CCN(CCCF)CC1c1ccc(C(=O)O)cc1]
or a pharmaceutically acceptable salt thereof.

43. A compound having the structure of 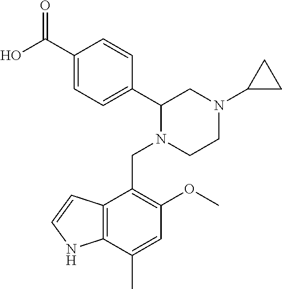 [SMILES: COc1cc(C)c2[nH]ccc2c1CN1CCN(C2CC2)CC1c1ccc(C(=O)O)cc1]
or a pharmaceutically acceptable salt thereof.

44. A compound having the structure of 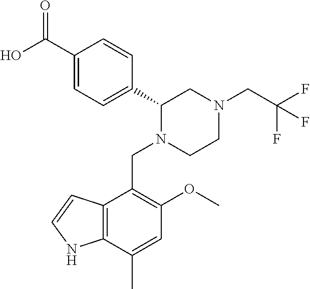 [SMILES: COc1cc(C)c2[nH]ccc2c1CN1CCN(CC(F)(F)F)C[C@H]1c1ccc(C(=O)O)cc1]
or a pharmaceutically acceptable salt thereof.

45. A compound having the structure of 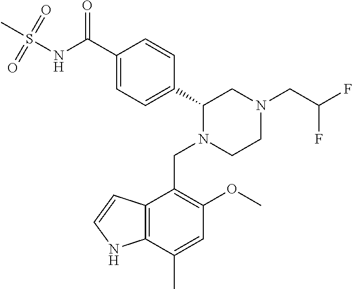 [SMILES: COc1cc(C)c2[nH]ccc2c1CN1CCN(CC(F)F)C[C@H]1c1ccc(C(=O)NS(C)(=O)=O)cc1]
or a pharmaceutically acceptable salt thereof.

46. The compound of any one of the preceding claims, wherein the compound has a diastereomeric, enantiomeric and/or isotopic purity of about or no less than about 90%, 91%, 92%, 93%, 94%, 95%, 96%, 97%, 98%, or 99%.

47. The compound of any one of the preceding claims, wherein the compound has a purity of about or no less than about 90%, 91%, 92%, 93%, 94%, 95%, 96%, 97%, 98%, or 99%.

48. A pharmaceutical composition comprising a compound of any one of the preceding claims or a pharmaceutically acceptable salt thereof, and a pharmaceutically acceptable carrier, excipient or diluent.

49. A pharmaceutical composition which delivers a compound of any one of the preceding claims or a pharmaceutically acceptable salt thereof.

50. A method, comprising removing one or more protection groups from a compound of formula M-I: 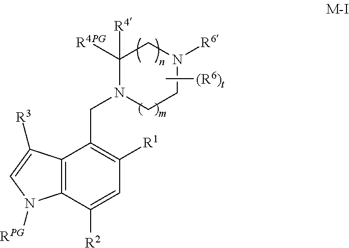
or a salt thereof to provide a compound of formula I or a salt thereof, or
a method comprising contacting an aldehyde or ketone with a compound of formula M-II: 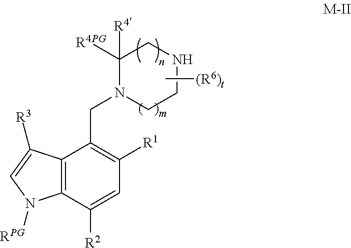
or a salt thereof in the presence of a reducing agent to provide a compound of formula M-I or a salt thereof, or
a method comprising reacting a compound of formula M-II: 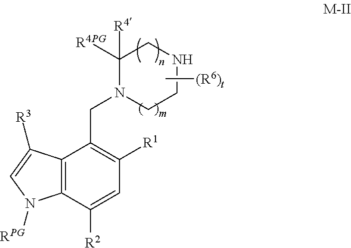
or a salt thereof with a compound having the structure of formula R^{6x3}—R^{6′}, wherein R^{6x3} is a leaving group; or
a method comprising reacting a compound of formula M-II: 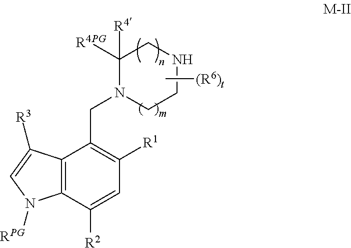
or a salt thereof with an acylating agent; or
a method comprising reacting a compound of formula M-II: 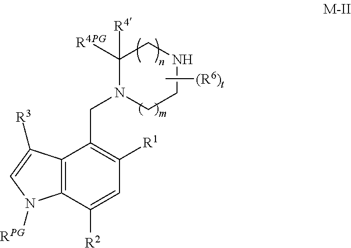
or a salt thereof with a sulfonylation agent; or
a method comprising de-protecting a compound having the structure of M-III: 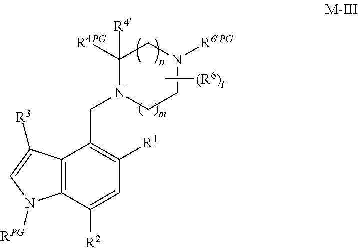
or a salt thereof to provide a compound of formula M-II or a salt thereof, or
a method comprising reacting a compound of formula M-IV: 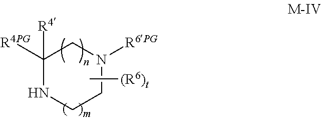
or a salt thereof, with a compound of formula M-V or a salt thereof: 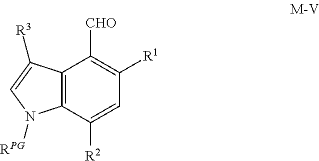
under a condition to provide a compound of formula M-III or a salt thereof, or
a method comprising protecting a group and/or de-protecting a group of a compound of formula M-VI: 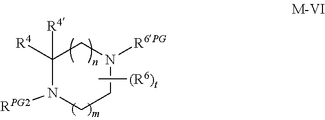
or a salt thereof to provide a compound of formula M-IV or a salt thereof, or
a method comprising converting a compound of formula M-VII: 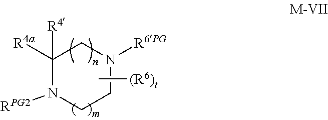
or a salt thereof to provide a compound of formula M-VI or a salt thereof; or
a method comprising protecting a group in a compound of formula M-VIII: 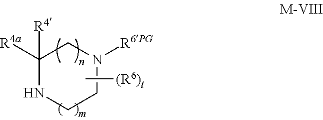
or a salt thereof to provide a compound of formula M-VII or a salt thereof; or
a method comprising converting a compound of formula M-IX: 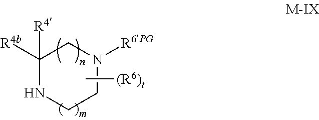
or a salt thereof to provide a compound of formula M-VIII or a salt thereof; or
a method comprising reducing a compound of formula M-X: 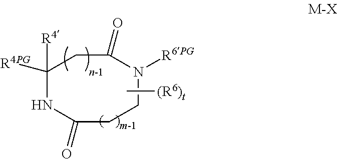
or a salt thereof to provide a compound of formula M-IX or a salt thereof, or
a method comprising converting a compound of formula M-XI: 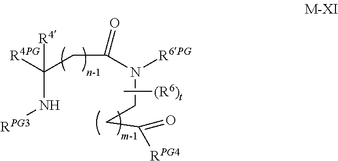
or a salt thereof to provide a compound of formula M-X or a salt thereof; or
a method comprising reacting a compound of formula M-XII: 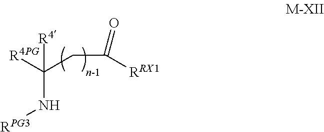
or a salt thereof, with a compound of formula M-XIII: 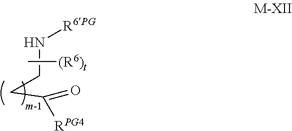
or a salt thereof to provide a compound of formula M-XI or a salt thereof, or
a method comprising converting a compound of formula M-XIV: 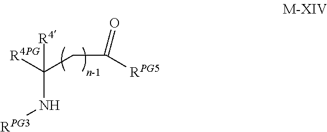
or a salt thereof to a compound of formula M-XII or a salt thereof, or
a method comprising reacting a compound of formula M-IV′: 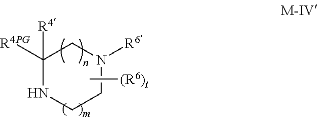
or a salt thereof, with a compound of formula M-V or a salt thereof: 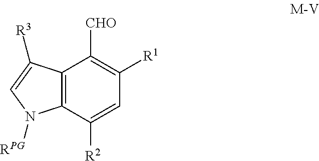
under a condition to provide a compound of formula M-I or a salt thereof, or
a method comprising de-protecting a group of a compound of formula M-XV: 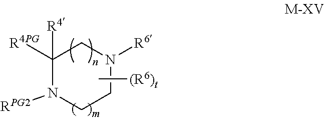
or a salt thereof to provide a compound of formula M-IV′ or a salt thereof; or
a method comprising converting compound of formula M-XVI: 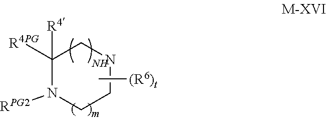
or a salt thereof to a compound of formula M-XV or a salt thereof, or
a method comprising de-protecting a group of a compound of formula M-XVII: 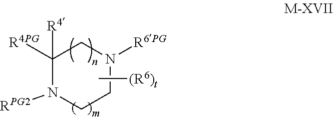
or a salt thereof to a compound of formula M-XVI or a salt thereof, or
a method comprising protecting a group of a compound of formula M-IV or a salt thereof to provide a compound of formula M-XVII or a salt thereof, or
a method for assessing a compound, comprising utilizing an assay described in the Examples.

51. A method of inhibiting a C3 convertase, comprising contacting a C3 convertase with a compound or composition of any one of the preceding claims; or
a method of modulating complement alternative pathway activity in a subject, wherein the method comprises administering to the subject a therapeutically effective amount of a compound or composition of any one of the preceding claims; or
a method of treating a disorder or a disease in a subject mediated by complement activation, wherein the method comprises administering to the subject a therapeutically effective amount of a compound or composition of any one of the preceding claims; or
a method of treating a disorder or a disease in a subject mediated by activation of the complement alternative pathway, wherein the method comprises administering to the subject a therapeutically effective amount of a compound or composition of any one of the preceding claims.

52. The method of claim 51, wherein the subject is suffering from a disease or disorder selected from age-related macular degeneration, geographic atrophy, Stargardt's disease, diabetic retinopathy, uveitis, glaucoma, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Creutzfeld-Jacob disease, Guillain Barre Syndrome, spinal cord injury, traumatic brain injury, Alzheimer's disease, Parkinson's disease, progressive supranuclear palsy, corticobasal syndrome, Pick's disease, mild cognitive impairment, Huntington's disease, diabetic neuropathy, neuropathic pain syndromes, fibromyalgia, frontotemporal dementia, dementia with Lewy bodies, multiple system atrophy, leptomeningeal metastasis, amyotrophic lateral sclerosis (ALS), chronic inflammatory demyelinating polyneuropathy (CIDP), neuromyelitis optica (NMO), disorders of inappropriate or undesirable complement activation, hemodialysis complications, graft rejection (e.g., hyperacute allograft rejection, xenograft rejection, interleukin-2 induced toxicity during IL-2 therapy, inflammatory disorders, inflammation of autoimmune diseases, Crohn's disease, acute respiratory distress syndrome (ARDS), myocarditis, postischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, asthma, chronic obstructive pulmonary disease (COPD), emphysema, pulmonary embolisms and infarcts, pneumonia, fibrogenic dust diseases, pulmonary fibrosis, rhinosinusitis, nasal polyposis, allergy, bronchoconstriction, hypersensitivity pneumonitis, parasitic diseases, Goodpasture's Syndrome, pulmonary vasculitis, pauci-immune vasculitis, thrombotic microangiopathy (TMA), immune complex-associated inflammation, antiphospholipid syndrome, glomerulonephritis, C3 glomerulopathy, IgA nephropathy, cancer, periodontitis, gingivitis, and obesity.

53. The method of claim 52, wherein the condition, disorder or disease is hemolytic anemia, and the subject is suffering from paroxysmal nocturnal hemoglobinuria.

54. The method of claim 52, wherein the condition, disorder or disease is hemolytic anemia, and the subject is suffering from autoimmune hemolytic anemia (e.g., cold agglutinin disease or warm autoimmune hemolytic anemia).

55. The method of claim 51, wherein the subject suffers from TMA secondary to atypical hemolytic uremic syndrome.

56. The method of claim 51, wherein the subject suffers from TMA secondary to hematopoietic stem cell transplant (HSCT-TMA).

57. The method of claim 51, wherein the subject suffers from drug-induced TMA.

58. The method of claim 51, wherein the subject suffers from complement activation secondary to administration of another therapeutic or diagnostic agent.

59. The method of claim 51, wherein the subject suffers from complement-mediated disorder is complement activation secondary to gene therapy (e.g., gene therapy with a viral vector such as an adeno-associated virus (AAV), adenovirus, or lentivirus vector).

60. The method of claim 51, wherein the subject suffers from complement-mediated disorder is complement activation secondary to cell therapy.

61. A method of treating age related macular degeneration, comprising administering to a subject suffering therefrom a therapeutically effective amount of a compound or composition of any one of the preceding claims.

62. The method of claim 61, wherein the age related macular degeneration is intermediate age-related macular degeneration.

63. The method of any one of claims 51-62, wherein another therapeutic agent is administered such that a subject is exposed to the effects of both the compound and the another therapeutic agent.

64. The method of any one of claims 51-63, wherein another therapeutic agent is administered prior to, concurrently with or subsequently to the administration of the compound.

65. A compound or composition of any one of the preceding claims, as and/or when used in the manufacture of a medicament for treating, or for use in the treatment of, a disorder or a disease in a subject mediated by complement activation or by activation of the complement alternative pathway.

66. The compound or composition of claim 65, wherein the subject is suffering from a disease or disorder selected from age-related macular degeneration, geographic atrophy, Stargardt's disease, diabetic retinopathy, uveitis, glaucoma, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Creutzfeld-Jacob disease, Guillain Barre Syndrome, spinal cord injury, traumatic brain injury, Alzheimer's disease, Parkinson's disease, progressive supranuclear palsy, corticobasal syndrome, Pick's disease, mild cognitive impairment, Huntington's disease, diabetic neuropathy, neuropathic pain syndromes, fibromyalgia, frontotemporal dementia, dementia with Lewy bodies, multiple system atrophy, leptomeningeal metastasis, amyotrophic lateral sclerosis (ALS), chronic inflammatory demyelinating polyneuropathy (CIDP), neuromyelitis optica (NMO), disorders of inappropriate or undesirable complement activation, hemodialysis complications, graft rejection (e.g., hyperacute allograft rejection, xenograft rejection, interleukin-2 induced toxicity during IL-2 therapy, inflammatory disorders, inflammation of autoimmune diseases, Crohn's disease, acute respiratory distress syndrome (ARDS), myocarditis, postischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, asthma, chronic obstructive pulmonary disease (COPD), emphysema, pulmonary embolisms and infarcts, pneumonia, fibrogenic dust diseases, pulmonary fibrosis, rhinosinusitis, nasal polyposis, allergy, bronchoconstriction, hypersensitivity pneumonitis, parasitic diseases, Goodpasture's Syndrome, pulmonary vasculitis, pauci-immune vasculitis, thrombotic microangiopathy (TMA), immune complex-associated inflammation, antiphospholipid syndrome, glomerulonephritis, C3 glomerulopathy, IgA nephropathy, cancer, periodontitis, gingivitis, and obesity.

67. The compound or composition of claim 66, wherein the condition, disorder or disease is hemolytic anemia, and the subject is suffering from paroxysmal nocturnal hemoglobinuria.

68. The compound or composition of claim 66, wherein the condition, disorder or disease is hemolytic anemia, and the subject is suffering from autoimmune hemolytic anemia (e.g., cold agglutinin disease or warm autoimmune hemolytic anemia).

69. The compound or composition of claim 65, wherein the subject suffers from TMA secondary to atypical hemolytic uremic syndrome.

70. The compound or composition of claim 65, wherein the subject suffers from TMA secondary to hematopoietic stem cell transplant (HSCT-TMA).

71. The compound or composition of claim 65, wherein the subject suffers from drug-induced TMA.

72. The compound or composition of claim 65, wherein the subject suffers from complement activation secondary to administration of another therapeutic or diagnostic agent.

73. The compound or composition of claim 65, wherein the subject suffers from complement-mediated disorder is complement activation secondary to gene therapy (e.g., gene therapy with a viral vector such as an adeno-associated virus (AAV), adenovirus, or lentivirus vector).

74. The compound or composition of claim 65, wherein the subject suffers from complement-mediated disorder is complement activation secondary to cell therapy.

75. A compound or composition of any one of the preceding claims, as and/or when used in the manufacture of a medicament for treating, or for use in the treatment of, age related macular degeneration.

76. The compound or composition of claim 75, wherein the age related macular degeneration is intermediate age-related macular degeneration.

77. The compound or composition of any one of claims 65-76, wherein another therapeutic agent is used such that a subject is exposed to the effects of both the compound or composition and the another therapeutic agent.

78. The compound or composition of any one of claims 65-77, wherein another therapeutic agent is administered prior to, concurrently with or subsequently to the administration of the compound.

79. A compound, composition, or method of any one of Example Embodiments 1-401 or as described in the specification.
